SEQUENCE LISTING (150 sequences)

SEQ ID NO: 1 - protein, 183 aa, Homo sapiens
  misc_feature (1)..(183): OX40L (TNSFR4) - Tumor necrosis factor ligand superfamily member 4 isoform 1
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 2 - protein, 133 aa, Homo sapiens
  misc_feature (1)..(133): OX40L (TNSFR4) - TNFSF4 isoform 2
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3 - protein, 198 aa, Mus musculus
  misc_feature (1)..(198): OX40L (TNSFR4) - TNFSF4
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 4 - RNA, 549 nt, Homo sapiens
  misc_feature (1)..(549): Human OX40L mRNA (ORF)
auggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuu

SEQ ID NO: 5 - RNA, 841 nt, Homo sapiens
  misc_feature: A,C G and U = AMP, CMP, GMP and  N1-Psi-UMP, respectively
  misc_feature (1)..(1): 5′7MeGpppG2′OMe (Me = methyl; p = inorganic phosphate)
  misc_feature (1)..(841): Full-length mRNA Nucleotide sequence (5 UTR, ORF, 3 UTR, polyA tail) of human OX40L
  misc_feature (841)..(841): -OH 3′
ggaaauaagagagaaaagaagaguaagaagaaauauaagagccaccauggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuuugauaauaggcuggagccucgguggccaugcuucuugccccuugggccuccccccagccccuccuccccuuccugcacccguaccccccaaacaccauugucacacuccaguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 6 - RNA, 549 nt, Homo sapiens; identical to SEQ ID NO: 4
  misc_feature (1)..(549): TNFSF4, ORF

SEQ ID NO: 7 - RNA, 3484 nt, Homo sapiens
  misc_feature (1)..(3484): OX40L - TNFSF4, transcript variant 1, mRNA NM_003326
ggcccugggaccuuugccuauuuucugauugauaggcuuuguuuugucuuuaccuccuucuuucuggggaaaacuucaguuuuaucgcacguuccccuuuuccauaucuucaucuucccucuacccagauugugaagauggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuuugaggggcugauggcaauaucuaaaaccaggcaccagcaugaacaccaagcuggggguggacagggcauggauucuucauugcaagugaaggagccucccagcucagccacgugggaugugacaagaagcagauccuggcccucccgcccccaccccucagggauauuuaaaacuuauuuuauauaccaguuaaucuuauuuauccuuauauuuucuaaauugccuagccgucacaccccaagauugccuugagccuacuaggcaccuuugugagaaagaaaaaauagaugccucuucuucaagaugcauuguuucuauuggucaggcaauugucauaauaaacuuaugucauugaaaacgguaccugacuaccauuugcuggaaauuugacauguguguggcauuaucaaaaugaagaggagcaaggagugaaggagugggguuaugaaucugccaaaggugguaugaaccaaccccuggaagccaaagcggccucuccaagguuaaauugauugcaguuugcauauugccuaaauuuaaacuuucucauuugguggggguucaaaagaagaaucagcuugugaaaaaucaggacuugaagagagccgucuaagaaauaccacgugcuuuuuuucuuuaccauuuugcuuucccagccuccaaacauaguuaauagaaauuucccuucaaagaacugucuggggaugugaugcuuugaaaaaucuaaucagugacuuaagagagauuuucuuguauacagggagagugagauaacuuauugugaaggguuagcuuuacuguacaggauagcagggaacuggacaucucaggguaaaagucaguacggauuuuaauagccuggggaggaaaacacauucuuugccacagacaggcaaagcaacacaugcucauccuccugccuaugcugagauacgcacucagcuccaugucuuguacacacagaaacauugcugguuucaagaaaugaggugauccuauuaucaaauucaaucugaugucaaauagcacuaagaaguuauugugccuuaugaaaaauaaugaucucugucuagaaauaccauagaccauauauagucucacauugauaauugaaacuagaagggucuauaaucagccuaugccagggcuucaauggaauaguauccccuuauguuuaguugaaauguccccuuaacuugauauaauguguuaugcuuauggcgcuguggacaaucugauuuuucaugucaacuuuccagaugauuuguaacuucucugugccaaaccuuuuauaaacauaaauuuuugagauauguauuuuaaaauuguagcacauguuucccugacauuuucaauagaggauacaacaucacagaaucuuucuggaugauucuguguuaucaaggaauuguacugugcuacaauuaucucuagaaucuccagaaagguggagggcuguucgcccuuacacuaaauggucucaguuggauuuuuuuuuccuguuuucuauuuccucuuaaguacaccuucaacuauauucccaucccucuauuuuaaucuguuaugaaggaagguaaauaaaaaugcuaaauagaagaaauuguagguaagguaagaggaaucaaguucugaguggcugccaaggcacucacagaaucauaaucauggcuaaauauuuauggagggccuacuguggaccaggcacugggcuaaauacuuacauuuacaagaaucauucugagacagauauucaaugauaucuggcuucacuacucagaagauuguguguguguuuguguguguguguguguguguauuucacuuuuuguuauugaccauguucugcaaaauugcaguuacucagugagugauauccgaaaaaguaaacguuuaugacuauagguaauauuuaagaaaaugcaugguucauuuuuaaguuuggaauuuuuaucuauauuucucacagaugugcagugcacaugcaggccuaaguauauguuguguguguuguuugucuuugaugucaugguccccucucuuaggugcucacucgcuuugggugcaccuggccugcucuucccauguuggccucugcaaccacacagggauauuucugcuaugcaccagccucacuccaccuuccuuccaucaaaaauaugugugugugucucagucccuguaagucauguccuucacagggagaauuaacccuucgauauacauggcagaguuuugugggaaaagaauugaaugaaaagucaggagaucagaauuuuaaauuugacuuagccacuaacuagccauguaaccuugggaaagucauuucccauuucugggucuugcuuuucuuucuguuaaaugagaggaauguuaaauaucuaacaguuuagaaucuuaugcuuacaguguuaucugugaaugcacauauuaaaugucuauguucuuguugcuaugagucaaggaguguaaccuucuccuuuacuauguugaauguauuuuuuucuggacaagcuuacaucuuccucagccaucuuugugaguccuucaagagcaguuaucaauuguuaguuagauauuuucuauuuagagaaugcuuaagggauuccaaucccgauccaaaucauaauuuguucuuaaguauacugggcagguccccuauuuuaagucauaauuuuguauuuagugcuuuccuggcucucagagaguauuaauauugauauuaauaauauaguuaauaguaauauugcuauuuacauggaaacaaauaaaagaucucagaauucacuaaaaaaaaaaa

SEQ ID NO: 8 - RNA, 737 nt, Homo sapiens
  misc_feature (1)..(737): mRNA sequence Human OX40L with 5′-UTR, 3′-UTR, and miR-122 binding site
gggaaauaagagagaaaagaagaguaagaagaaauauaagagccaccauggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuuugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 9 - RNA, 549 nt, Homo sapiens
  misc_feature (1)..(549): mRNA open reading frame sequence 1 for Human OX40L
auggagcgggugcagccccuggaggagaacgugggcaacgccgcucggccacgguucgagcggaacaagcugcugcugguggcuagcgugauccagggccugggccugcugcugugcuucaccuacaucugccugcacuucagcgcccugcaggugagccaccgguauccccggauccagagcaucaaggugcaguucaccgaguacaagaaggagaagggcuucauccugaccagccagaaggaggacgagaucaugaaggugcagaacaacagcgugaucaucaacugcgacggcuucuaccugaucagccugaagggcuacuucagccaggaggugaacaucagccugcacuaccagaaggacgaggagccccuguuccagcugaagaaggugcggagcgugaacagccugaugguggccagccugaccuacaaggacaagguguaccugaacgugaccaccgacaacaccagccuggacgacuuccacgugaacggcggcgagcugauccugauccaccagaaccccggcgaguucugcgugcug

SEQ ID NO: 10 - RNA, 549 nt, Homo sapiens
  misc_feature (1)..(549): mRNA open reading frame sequence 2 for Human OX40L
auggaaaggguccaaccccucgaagagaacgugggaaacgcagccaggccaagauucgagaggaacaagcuauugcucguggccucaguaauucagggacucggguuacuccuuugcuucaccuacaucugcuugcacuucagugcucugcagguaucacaucgguauccucgaauucaaaguaucaaaguacaauuuaccgaauauaagaaggagaaagguuucauccucacuucccagaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguagccucucugacuuacaaagacaaagucuacuugaaugugaccacugacaauaccucccuggaugacuuccaugugaauggcggagaacugauucuuauccaucagaauccuggugaauucuguguccuu

SEQ ID NO: 11 - RNA, 549 nt, Homo sapiens; identical to SEQ ID NO: 9
  misc_feature (1)..(549): mRNA open reading frame sequence 3 for Human OX40L

SEQ ID NO: 12 - RNA, 57 nt, Artificial Sequence
  Synthetic 5UTR
aggaaauaagagagaaaagaagaguaagaagaaauauaagaccccggcgccgccacc

SEQ ID NO: 13 - protein, 267 aa, Homo sapiens
  misc_feature (1)..(267): Full Length IL15R Amino Acid Sequence (Wild Type)
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 14 - RNA, 801 nt, Homo sapiens
  misc_feature (1)..(801): Full-Length IL15R mRNA ORF #1
auggcuccccgccgcgcgcgaggcugucgcacccucggacuuccugcacucuugcuuuugcuccuccuuagacccccugcaaccagagggauaaccuguccaccuccaaugagcgucgagcacgcagacauuugggugaaaucauacagucuguacaguagagagcgguacaucugcaacaguggguuuaaaagaaaagcaggcacuucaucucugacagagugcgugcugaacaaagcaacuaauguagcucauuggacaaccccaucacugaaaugcauuagagauccagcucuggugcaucaaagaccagcaccaccaaguaccguaacaaccgcaggggugaccccucagccugagucccuaucucccuccggcaaggagccagcagcaucuucaccuagcuccaauaacaccgcagcuaccacugccgccauaguccccggaagccagcugaugccuaguaaaucucccucaacagguaccaccgaaauuucuagccaugaguccucgcacggcaccccgucacagacuacagcuaaaaacugggagcuaacggcuucggcaucccaccaaccuccaggcguuuauccccaaggucacuccgacacuacuguggcgauuagcacaaguaccguccuucuguguggacugagugcaguuucauugcuggccuguuaucugaaaucucgccagaccccuccccucgccaguguugagauggaagccauggaagcacuuccugugacuuggggaacauccucgagggacgaggaccucgagaacugcucucaccaccug

SEQ ID NO: 15 - protein, 162 aa, Homo sapiens
  misc_feature (1)..(162): Full-Length IL15 Amino Acid Sequence (Wild Type)
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 16 - RNA, 486 nt, Homo sapiens
  misc_feature (1)..(486): Full-length IL15 Nucleotide Sequence (Wild Type)
augcgcaucagcaaaccucauuuacggaguaucagcauccagugcuaucucugccugcuucugaacagucauuuucugacugaggcgggcauucaugucuuuauuuuaggcugcuuuuccgcaggucugcccaaaacagaagcaaauugggugaacgugaucagcgaccugaagaagauugaggaucuaauucaaagcaugcauauugaugccacacucuacaccgaauccgacgugcacccuucguguaaagugacugcaaugaaguguuucuuacuggaacugcaggugaucagucuggaguccggggaugcaucaauccacgacacaguggaaaaccugauuauccuggcaaacaauucccugagcaguaauggcaaugucacggagagcggauguaaggagugugaggaauuagaggaaaagaauaucaaggaauuccuucaguccuuugugcacaucguacagauguuuauuaauacaucc

SEQ ID NO: 17 - protein, 149 aa, Homo sapiens
  misc_feature (1)..(149): tPA-IL15 Amino acid Sequence
MDAMKRGLCCVLLLCGAVFVSPSQEIHARFRRGARNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 18 - protein, 463 aa, Artificial Sequence
  Synthetic Full Fc-IL15R-Linker-IL15 Amino acid Sequence
METDTLLLWVLLLWVPGSTGEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 19 - RNA, 447 nt, Homo sapiens
  misc_feature (1)..(447): HS IL15opt-tPA6 mRNA ORF #1
auggaugcaaugaagagagggcucugcugugugcugcugcuguguggagcagucuucguuucgcccagccaggaaauccaugcccgauucagaagaggagccagaaacugggugaacgugaucucggaccugaagaagaucgaggaccucauccagucgaugcacaucgacgcgacgcuguacacggagucggacguccacccgucgugcaaggucacggcgaugaagugcuuccuccuggagcuccaagucaucucgcucgagucgggggacgcgucgauccacgacacgguggagaaccugaucauccuggcgaacaacucgcugucgucgaacgggaacgucacggagucgggcugcaaggagugcgaggagcuggaggagaagaacaucaaggaguuccugcagucguucgugcacaucguccagauguucaucaacacgucg

SEQ ID NO: 20 - RNA, 447 nt, Homo sapiens
  misc_feature (1)..(447): Hs IL15opt-tPA6 mRNA ORF #3
auggacgccaugaagcgcggccugugcugcgugcugcugcugugcggcgccguguucgugagccccucccaggagauccacgcaagguucaggaggggcgccagaaacugggugaacgugaucucugaucugaagaagaucgaggaucucauacagagcaugcacaucgacgcaacccuguacaccgagagcgacgugcacccuagcugcaaggugacagcuaugaagugcuuccugcucgagcugcaggugaucagccuggagagcggcgacgccuccauucacgacaccguggagaaccugaucauccuggccaauaacagccugagcagcaacggcaacgugacagaaagcggcuguaaggagugcgaggagcuggaggagaagaacaucaaagaguuucugcagagcuucgugcacauugugcagauguucaucaacaccagc

SEQ ID NO: 21 - RNA, 801 nt, Homo sapiens
  misc_feature (1)..(801): Hs IL15Ra mRNA ORF #2
auggcaccaagacgcgccaggggcugucgcacccucggacuuccugcacucuugcuuuugcuccuccuuagaccaccugcaaccagagggauaaccuguccaccuccaaugagcgucgagcacgcagacauuugggugaaaucauacagucuguacaguagagagcgguacaucugcaacaguggguuuaagaggaaggcaggcacuucaucucugacagagugcgugcugaacaaagcaacuaacguagcucauuggacaaccccaucacugaagugcauuagagauccagcucuggugcaucaaagaccagcaccaccaaguaccguaacaaccgcaggggugaccccucagccugagucccuaucucccuccggcaaggagccagcagcaucuucaccuagcuccaauaacaccgcagcuaccacugccgccauaguccccggaagccagcugaugccuaguaaaucucccucaacagguaccaccgaaauuucuagccacgaguccucgcacggcaccccgucacagacuacagcuaagaacugggagcuaacggcuucggcaucccaccaaccuccaggcguuuauccccaaggucacuccgacacuacuguggcgauuagcacaaguaccguccuucuguguggacugagugcaguuucauugcuggccuguuaucugaaaucucgccagaccccuccccucgccaguguugagauggaagccauggaagcacuuccugugacuuggggaacauccucgagggacgaggaccucgagaacugcucucaccaccug

SEQ ID NO: 22 - RNA, 801 nt, Homo sapiens
  misc_feature (1)..(801): Hs IL15Ra mRNA ORF #3
auggccccuagacgggccaggggcugcaggacacugggccugcccgcccugcugcuccugcuguugcucagaccucccgccaccagaggaaucaccugcccuccucccaugagcguggagcacgccgacaucugggugaaguccuacagccuguacagccgggagagauacaucuguaacuccggcuucaagagaaaggccggcaccagcucccugaccgagugugugcugaacaaggccaccaacguggcccacuggaccacacccagccugaagugcaucagagaucccgcucuggugcaccaaagaccugcuccuccuagcaccgugaccacagccggcgugacaccucaaccggaaagccugagccccuccggcaaggagccggccgccuccucacccagcagcaacaacaccgccgccacaaccgccgcuaucgugcccggcagccagcugaugcccagcaagagccccagcacaggaacaaccgagaucagcucucacgaauccagccacggcaccccuagccagacaaccgcuaagaacugggagcugacagccagcgcuagccaccagccacccgggguguacccacagggccacagcgacaccaccguggccaucagcaccagcacugugcugcugugcgggcucagcgcagugagccugcuggcuugcuaccuuaaguccagacagaccccuccccuggcuuccgucgaaauggaggccauggaggcucugccugugaccuggggcacaagcagcagggacgaggaucuggagaacugcucucaccaccug

SEQ ID NO: 23 - protein, 395 aa, Artificial Sequence [sequence omitted]

SEQ ID NO: 24 - RNA, 1185 nt, Artificial Sequence
  Synthetic Nucleotide Sequence #1 Hs IL15opt-IgEsp_IL15Ra
auggacuggaccuggauccuguuccugguggccgccgccaccagagugcacagcaacugggugaacgugaucucggaccugaagaagaucgaggaccucauccagucgaugcacaucgacgcgacgcuguacacggagucggacguccacccgucgugcaaggucacggcgaugaagugcuuccuccuggagcuccaagucaucucgcucgagucgggcgacgcgucgauccacgacacgguggagaaccugaucauccuggcgaacaacucgcugucgucgaacgggaacgucacggagucgggcugcaaggagugcgaggagcuggaggagaagaacaucaaggaguuccugcagucguucgugcacaucguccagauguucaucaacacgucgucugguggcggaucaggugguggcggaucuggcggugguggaaguggagguggcgggucuggcggagguucacugcagauaaccuguccaccuccaaugagcgucgagcacgcagacauuugggugaaaucauacagucuguacaguagagagcgguacaucugcaacaguggguuuaagagaaaggcaggcacuucaucucugacagagugcgugcugaacaaagcaacuaauguagcucauuggacaaccccaucacugaaaugcauuagagauccagcucuggugcaucaaagaccagcaccaccaaguaccguaacaaccgcaggggugaccccucagccugagucccuaucucccuccggcaaggagccagcagcaucuucaccuagcuccaauaacaccgcagcuaccacugccgccauaguccccggaagccagcugaugccuaguaaaucucccucaacagguaccaccgaaauuucuagccaugaguccucgcacggcaccccgucacagacuacagcuaagaacugggagcuaacggcuucggcaucccaccaaccuccaggcguuuauccccaaggucacuccgacacuacuguggcgauuagcacaaguaccguccuucuguguggacugagugcaguuucauugcuggccuguuaucugaaaucucgccagaccccuccccucgccaguguugagauggaagccauggaagcacuuccugugacuuggggaacauccucgagggacgaggaccucgagaacugcucucaccaccug

SEQ ID NO: 25 - RNA, 1185 nt, Artificial Sequence
  Synthetic Nucleotide Sequence #2 Hs IL15opt-IgEsp_IL15Ra
auggacuggaccuggauccuguuccugguggcugccgccaccagggugcacagcaacugggugaacgugaucucggaccugaagaagaucgaggaccucauccagucgaugcacaucgacgcgacgcuguacacggagucggacguccacccgucgugcaaggucacggcgaugaagugcuuccuccuggagcuccaagucaucucgcucgagucgggcgacgcgucgauccacgacacgguggagaaccugaucauccuggcgaacaacucgcugucgucgaacgggaacgucacggagucgggcugcaaggagugcgaggagcuggaggagaagaacaucaaggaguuccugcagucguucgugcacaucguccagauguucaucaacacgucgagcggcggcgguaguggaggagguggcuccgguggaggcggcagcgguggcgguggaaguggcggcggcagccugcagaucaccuguccuccucccaugagcguggagcacgccgacaucugggugaagagcuacagccuguacagccgggagcgguacaucugcaacagcggcuucaagcggaaggccggcacaagcagccugaccgagugcgugcugaacaaggccaccaacguggcccacuggaccaccccaagccugaagugcauccgggaccccgcccuggugcaucagcggcccgccccaccuagcaccgugaccaccgccggcgugacaccccagcccgagagccugagccccagcggcaaggagcccgcugccagcuccccaagcagcaacaacaccgccgccacaaccgccgccaucgugcccggcagccagcugaugcccagcaagagccccagcaccggcaccaccgagaucagcagccacgagagcagccacggcacacccagccagaccaccgccaagaacugggagcugaccgccagcgccagccaccagccucccggcguguauccccagggccacagcgacaccaccguggccaucagcaccagcaccgugcugcugugcggccugagcgccgugagccugcuggccugcuaccugaagagccggcagaccccaccccuggccagcguggagauggaggccauggaggcccugcccgugaccuggggcaccagcagccgggacgaggaccuggagaacugcagccaccaccug

SEQ ID NO: 26 - RNA, 1185 nt, Artificial Sequence
  Synthetic Nucleotide Sequence #3 Hs IL15opt-IgEsp_IL15Ra
auggacuggaccuggauccuguuucucguugccgccgcuaccagagugcacagcaacugggugaacgugaucagcgaccugaagaagaucgaggaccugauccagagcaugcacaucgacgccacccuguacaccgagagcgacgugcaccccagcugcaaggugaccgccaugaagugcuuccugcuggagcugcaggugaucagccuggagagcggcgacgccagcauccacgacaccguggagaaccugaucauccuggccaacaacagccugagcagcaacggcaacgugaccgagagcggcugcaaggagugcgaggagcuggaggagaagaacaucaaggaguuccugcagagcuucgugcacaucgugcagauguucaucaacaccagcagugguggcggaaguggcggcggaggcaguggaggcggcggcucaggaggcgguggcucaggcggagguagccugcagaucaccugcccaccucccaugagcguggagcacgccgacaucugggugaagagcuacagccuguacagccgggagcgguacaucugcaacagcggcuucaagcggaaggccggcaccagcagccugaccgagugcgugcugaacaaggccaccaacguggcccacuggacaaccccuagccugaagugcauuagagacccggcccuggugcaucaacggcccgccccaccuagcaccgugaccaccgccggcgugacuccccagcccgagagccugagccccagcggcaaggaacccgccgccaguagccccagcagcaacaacacagccgccacuaccgccgccaucgugcccggcagccagcugaugcccagcaagagccccagcaccggcaccaccgagaucagcagccacgagagcagccacggcaccccuagccagaccaccgccaagaacugggagcugaccgccagcgccagccaccaaccaccaggcguguacccucagggccacagcgacaccaccguggccaucagcaccagcaccgugcugcugugcggccugagcgccgugagccugcuggccugcuaccugaagagccggcagaccccaccccuggccagcguggagauggaggccauggaggcccugcccgugaccuggggcaccagcucucgggacgaggaccuggagaacugcagccaccaccug

SEQ ID NO: 27 - protein, 412 aa, Artificial Sequence
  Synthetic Hs IL15opt-tPA6_IL15Ra WT
MDAMKRGLCCVLLLCGAVFVSPSQEIHARFRRGARNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 28 - RNA, 1236 nt, Artificial Sequence
  Synthetic Nucleotide Sequence #1 Hs IL15opt-tPA6_IL15Ra WT
auggaugcaaugaagagagggcucugcugugugcugcugcuguguggagcagucuucguuucgcccagccaggaaauccaugcccgauucagaagaggagccagaaacugggugaacgugaucucggaccugaagaagaucgaggaccucauccagucgaugcacaucgacgcgacgcuguacacggagucggacguccacccgucgugcaaggucacggcgaugaagugcuuccuccuggagcuccaagucaucucgcucgagucgggcgacgcgucgauccacgacacgguggagaaccugaucauccuggcgaacaacucgcugucgucgaacgggaacgucacggagucgggcugcaaggagugcgaggagcuggaggagaagaacaucaaggaguuccugcagucguucgugcacaucguccagauguucaucaacacgucgucugguggcggaucaggugguggcggaucuggcggugguggaaguggagguggcgggucuggcggagguucacugcagauaaccuguccaccuccaaugagcgucgagcacgcagacauuugggugaaaucauacagucuguacaguagagagcgguacaucugcaacaguggguuuaagagaaaggcaggcacuucaucucugacagagugcgugcugaacaaagcaacuaauguagcucauuggacaaccccaucacugaaaugcauuagagauccagcucuggugcaucaaagaccagcaccaccaaguaccguaacaaccgcaggggugaccccucagccugagucccuaucucccuccggcaaggagccagcagcaucuucaccuagcuccaauaacaccgcagcuaccacugccgccauaguccccggaagccagcugaugccuaguaaaucucccucaacagguaccaccgaaauuucuagccaugaguccucgcacggcaccccgucacagacuacagcuaagaacugggagcuaacggcuucggcaucccaccaaccuccaggcguuuauccccaaggucacuccgacacuacuguggcgauuagcacaaguaccguccuucuguguggacugagugcaguuucauugcuggccuguuaucugaaaucucgccagaccccuccccucgccaguguugagauggaagccauggaagcacuuccugugacuuggggaacauccucgagggacgaggaccucgagaacugcucucaccaccug

SEQ ID NO: 29 - RNA, 1236 nt, Artificial Sequence
  Synthetic Nucleotide Sequence #2 Hs IL15-tPA6_IL15Ra-opt
auggaugcaaugaagagagggcucugcugugugcugcugcuguguggagcagucuucguuucgcccagccaggaaauccaugcccgauucagaagaggagccagaaacugggugaacgugaucucggaccugaagaagaucgaggaccucauccagucgaugcacaucgacgcgacgcuguacacggagucggacguccacccgucgugcaaggucacggcgaugaagugcuuccuccuggagcuccaagucaucucgcucgagucgggcgacgcgucgauccacgacacgguggagaaccugaucauccuggcgaacaacucgcugucgucgaacgggaacgucacggagucgggcugcaaggagugcgaggagcuggaggagaagaacaucaaggaguuccugcagucguucgugcacaucguccagauguucaucaacacgucgagcggcggcgguaguggaggagguggcuccgguggaggcggcagcgguggcgguggaaguggcggcggcagccugcagaucaccuguccuccucccaugagcguggagcacgccgacaucugggugaagagcuacagccuguacagccgggagcgguacaucugcaacagcggcuucaagcggaaggccggcacaagcagccugaccgagugcgugcugaacaaggccaccaacguggcccacuggaccaccccaagccugaagugcauccgggaccccgcccuggugcaucagcggcccgccccaccuagcaccgugaccaccgccggcgugacaccccagcccgagagccugagccccagcggcaaggagcccgcugccagcuccccaagcagcaacaacaccgccgccacaaccgccgccaucgugcccggcagccagcugaugcccagcaagagccccagcaccggcaccaccgagaucagcagccacgagagcagccacggcacacccagccagaccaccgccaagaacugggagcugaccgccagcgccagccaccagccucccggcguguauccccagggccacagcgacaccaccguggccaucagcaccagcaccgugcugcugugcggccugagcgccgugagccugcuggccugcuaccugaagagccggcagaccccaccccuggccagcguggagauggaggccauggaggcccugcccgugaccuggggcaccagcagccgggacgaggaccuggagaacugcagccaccaccug

SEQ ID NO: 30 - RNA, 1236 nt, Artificial Sequence
  Synthetic Nucleotide Sequence #3 Hs IL15-15Ra_tPA6_fullopt
auggacgccaugaagcggggccugugcugcgugcugcugcugugcggcgccguguucgugagccccagccaggagauccacgcccguuuccgacggggcgccagaaacugggugaacgugaucagcgaccugaagaagaucgaggaccugauccagagcaugcacaucgacgccacccuguacaccgagagcgacgugcaccccagcugcaaggugaccgccaugaagugcuuccugcuggagcugcaggugaucagccuggagagcggcgacgccagcauccacgacaccguggagaaccugaucauccuggccaacaacagccugagcagcaacggcaacgugaccgagagcggcugcaaggagugcgaggagcuggaggagaagaacaucaaggaguuccugcagagcuucgugcacaucgugcagauguucaucaacaccagcucuggcggcggcucaggaggaggaggcagcggaggaggcgguagcggcggagguggaagcggcggcggaagccugcagaucaccuguccgccucccaugagcguggagcacgccgacaucugggugaagagcuacagccuguacagccgggagcgguacaucugcaacagcggcuucaagcggaaggccgguaccagcagucugaccgagugcgugcugaacaaggccaccaacguggcccacuggaccaccccaagccugaagugcauccgcgacccagcccuggugcaucagcggcccgcuccuccaagcaccgugaccaccgcuggcgugaccccacagcccgagagccuguccccaagcggcaaggagcccgccgcuagcagccccagcagcaacaacacagccgccaccaccgcugccaucgugcccggcagccagcugaugcccagcaagagccccagcaccggcaccaccgagaucagcagccacgagagcagccacggcaccccuagccagaccaccgccaagaacugggagcugaccgccagcgccagccaccagccaccuggcguguauccccagggccacagcgacaccaccguggccaucagcaccagcaccgugcugcuguguggccugagcgccgugagccugcuggccugcuaccugaagucccgccaaaccccgccacuggccagcguggagauggaggccauggaggcccugcccgugaccuggggcaccagcagccgggacgaggaccuggagaacugcagccaccaccug

SEQ ID NO: 31 - protein, 239 aa, Artificial Sequence
  Synthetic Hs IL-15 RLI
METDTLLLWVLLLWVPGSTGDYKDDDDKITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 32 - RNA, 717 nt, Artificial Sequence
  Synthetic Hs IL15-RLI mRNA open reading frame
auggaaaccgacacucugcugcuuugggugcugcugcucugggugcccggauccaccggagauuacaaggacgaugacgacaagaucaccuguccaccucccauguccguggaacacgccgacaucugggucaaaagcuacagccuguacucccgggaaagauacauuuguaacagcggguuuaagaggaaggccggcaccuccucguugaccgaaugcgugcugaacaaggcuaccaacguggcccauuggacuaccccgucccugaagugcauucgcgauccugcccucgugcaccaacggccugcgccgccguccggagggucuggcggugguggaucgggaggcgguucaggaggcggagggucgcugcagaacugggugaacgugaucuccgaccuuaagaagaucgaggaccugauccaguccaugcacaucgacgcaacccuguauacugagucagacgugcaccccuccugcaaagucacagcgaugaagugcuuccugcuggaacuccaggucaucuccuuggaaucgggagaugccuccauucacgacacugucgagaaccucauuauucuggccaacaacagccuguccagcaauggcaacgugacggagucaggcugcaaggagugcgaggaacucgaagagaagaauaucaaggaguuccugcagucguucgugcauaucgugcaaauguucaucaacaccagc

SEQ ID NO: 33 - protein, 306 aa, Artificial Sequence
  Synthetic Wildtype IL12B without signal amino acids
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 34 - RNA, 918 nt, Artificial Sequence
  Synthetic Wildtype IL12B without signal nucleic acids
auaugggaacugaagaaagauguuuaugucguagaauuggauugguauccggaugccccuggagaaauggugguccucaccugugacaccccugaagaagaugguaucaccuggaccuuggaccagagcagugaggucuuaggcucuggcaaaacccugaccauccaagucaaagaguuuggagaugcuggccaguacaccugucacaaaggaggcgagguucuaagccauucgcuccugcugcuucacaaaaaggaagauggaauuugguccacugauauuuuaaaggaccagaaagaacccaaaaauaagaccuuucuaagaugcgaggccaagaauuauucuggacguuucaccugcugguggcugacgacaaucaguacugauuugacauucagugucaaaagcagcagaggcucuucugacccccaaggggugacgugcggagcugcuacacucucugcagagagagucagaggggacaacaaggaguaugaguacucaguggagugccaggaggacagugccugcccagcugcugaggagagucugcccauugaggucaugguggaugccguucacaagcucaaguaugaaaacuacaccagcagcuucuucaucagggacaucaucaaaccugacccacccaagaacuugcagcugaagccauuaaagaauucucggcagguggaggucagcugggaguacccugacaccuggaguacuccacauuccuacuucucccugacauucugcguucagguccagggcaagagcaagagagaaaagaaagauagagucuucacggacaagaccucagccacggucaucugccgcaaaaaugccagcauuagcgugcgggcccaggaccgcuacuauagcucaucuuggagcgaaugggcaucugugcccugcagu

SEQ ID NO: 35 - protein, 197 aa, Artificial Sequence
  Synthetic Wildtype IL12A without signal amino acids
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 36 - RNA, 591 nt, Artificial Sequence
  Synthetic Wildtype IL12A without signal nucleic acids
agaaaccuccccguggccacuccagacccaggaauguucccaugccuucaccacucccaaaaccugcugagggccgucagcaacaugcuccagaaggccagacaaacucuagaauuuuacccuugcacuucugaagagauugaucaugaagauaucacaaaagauaaaaccagcacaguggaggccuguuuaccauuggaauuaaccaagaaugagaguugccuaaauuccagagagaccucuuucauaacuaaugggaguugccuggccuccagaaagaccucuuuuaugauggcccugugccuuaguaguauuuaugaagacuugaagauguaccagguggaguucaagaccaugaaugcaaagcuucugauggauccuaagaggcagaucuuucuagaucaaaacaugcuggcaguuauugaugagcugaugcaggcccugaauuucaacagugagacugugccacaaaaauccucccuugaagaaccggauuuuuauaaaacuaaaaucaagcucugcauacuucuucaugcuuucagaauucgggcagugacuauugauagagugaugagcuaucugaaugcuucc

SEQ ID NO: 37 - protein, 22 aa, Artificial Sequence
  Synthetic Wildtype IL12B signal peptide amino acids
MCHQQLVISWFSLVFLASPLVA

SEQ ID NO: 38 - RNA, 66 nt, Artificial Sequence
  Synthetic Wildtype IL12B signal peptide nucleic acids
augugucaccagcaguuggucaucucuugguuuucccugguuuuucuggcaucuccccucguggcc

SEQ ID NO: 39 - protein, 532 aa, Artificial Sequence
  Synthetic Signal peptide-IL12B-linker-IL12A amino acid sequence #1
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 40 - protein, 533 aa, Artificial Sequence
  Synthetic Signal peptide-IL12B-linker-IL12A amino acid sequence #2
MGCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 41 - protein, 25 aa, Homo sapiens
  misc_feature (1)..(25): Human CD8A transmembrane domain
IYIWAPLAGTCGVLLLSLVITLYCY

SEQ ID NO: 42 - protein, 25 aa, Homo sapiens
  misc_feature (1)..(25): Human PDGF-RB transmembrane domain
VVVISAILALVVLTIISLIILIMLW

SEQ ID NO: 43 - protein, 21 aa, Homo sapiens
  misc_feature (1)..(21): Human CD80 Transmembrane domain
LLPSWAITLISVNGIFVICCL

SEQ ID NO: 44 - RNA, 1839 nt, Artificial Sequence
  Synthetic mIL12AB-PTM_v5 miR122 Nucleotide Sequence
auguguccucagaagcuaaccaucuccugguuugccaucguuuugcuugugucuccacucauggccaugugggagcucgagaaagacguuuacguuguagagguggacuggacucccgacgccccaggagaaacagugaaccucaccugugacacgccugaagaagaugacaucaccuggaccucagaccagagacauggagucauaggcucuggaaagacccugaccaucacugucaaagaguuccuagaugcuggccaguacaccugccacaaaggaggcgagacucugagccacucacaucugcugcuccacaagaaggagaauggaauuugguccaccgaaauccugaagaacuucaagaauaagacuuuccugaagugugaagcaccaaauuacuccggacgguucacgugcucauggcuggugcaaagaaacauggacuugaaguucaacaucaagagcaguagcaguuccccugacucucgggcagugacauguggaauggcgucucugucugcagagaaggucacacuggaccaaagggacuaugagaaguauucaguguccugccaggaggaugucaccugcccaacugccgaggaaacucugcccauugaacuggcguuggaagcacggcagcagaauaaauaugagaacuacagcaccagcuucuucaucagggacaucaucaaaccagacccgcccaagaacuugcagaugaagccuuugaagaacucacagguggaggucagcugggaguacccagacuccuggagcacuccccauuccuacuucucccucaaguucuuuguucgaauccagcgcaagaaagagaagaugaaggagacagaggagggguguaaccagaaaggugcguuccucguagagaagacaucuaccgaaguccaaugcaaaggcgggaaugucugcgugcaagcucaggaucgcuauuacaauuccucaugcagcaagugggcauguguucccugcaggguccgauccggaggcggagggucuggaggaggagguucuggaggugguggcaguagggucauuccagucucuggaccugcaaggugucuuagccagucccgaaaccugcugaagaccacagacgauauggugaagacggccagagagaaacugaaacauuauuccugcacagcagaggacaucgaucaugaagauauuacacgggaccaaaccagcacauugaagaccuguuuaccacuggaacuacacaagaacgagaguugccuggcuacuagagagacuucuuccacaacaagagggagcugccugccaccacagaagacgucuuugaugaugacccugugccuugguagcaucuacgaggaucucaagauguaccagacagaguuccaggccaucaacgcagcacuucagaaucacaaccaucagcagaucauuuuagacaagggcaugcugguggccaucgaugagcugaugcaaucucugaaucauaauggcgagacacuucgccagaaaccuccugugggagaagcagacccuuacagagugaagaugaagcucugcauccugcuucacgccuucagcacccgcgucgucacuauuaacagggugaugggcuaucugagcuccgccucugguggcggaucaggcggcggcggcucuggcggcgguggaagcggagguggcgggucuggcggagguucacugcagguaguagugaucagcgccauccuggcccugguggugcugaccgugaucucauugaucaucuugauuaugcuguggggcggaggaggcagcgggaaaccaauuccaaauccccuccugggguuggauagcacc

SEQ ID NO: 45 - protein, 613 aa, Artificial Sequence
  Synthetic mIL12AB-PTM_v5 miR122 Amino Acid Sequence
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGGSGGGGSGGGGSGGGGSGGGSLQVVVISAILALVVLTVISLIILIMLWGGGGSGKPIPNPLLGLDST

SEQ ID NO: 46 - RNA, 1596 nt, Artificial Sequence
  Synthetic hIL12AB_041 mRNA ORF
augugccaccagcagcuggugaucagcugguucagccugguguuccuggccagcccccugguggccaucugggagcugaagaaggacguguacgugguggaguuggauugguaccccgacgcccccggcgagaugguggugcugaccugcgacacccccgaggaggacggcaucaccuggacccuggaccagagcagcgaggugcugggcagcggcaagacccugaccauccaggugaaggaguucggcgacgccggccaguacaccugccacaagggcggcgaggugcugagccacagccugcugcugcugcacaagaaggaggacggcaucuggagcaccgacauccugaaggaccagaaggagcccaagaacaagaccuuccugagaugcgaggccaagaacuacagcggcagauucaccugcugguggcugaccaccaucagcaccgaccugaccuucagcgugaagagcagcagaggcagcagcgacccccagggcgugaccugcggcgccgccacccugagcgccgagagagugagaggcgacaacaaggaguacgaguacagcguggagugccaggaagauagcgccugccccgccgccgaggagagccugcccaucgaggugaugguggacgccgugcacaagcugaaguacgagaacuacaccagcagcuucuucaucagagauaucaucaagcccgacccccccaagaaccugcagcugaagccccugaagaacagccggcagguggaggugagcugggaguaccccgacaccuggagcaccccccacagcuacuucagccugaccuucugcgugcaggugcagggcaagagcaagagagagaagaaagauagaguguucaccgacaagaccagcgccaccgugaucugcagaaagaacgccagcaucagcgugagagcccaagauagauacuacagcagcagcuggagcgagugggccagcgugcccugcagcggcggcggcggcggcggcagcagaaaccugcccguggccacccccgaccccggcauguuccccugccugcaccacagccagaaccugcugagagccgugagcaacaugcugcagaaggcccggcagacccuggaguucuaccccugcaccagcgaggagaucgaccacgaagauaucaccaaagauaagaccagcaccguggaggccugccugccccuggagcugaccaagaacgagagcugccugaacagcagagagaccagcuucaucaccaacggcagcugccuggccagcagaaagaccagcuucaugauggcccugugccugagcagcaucuacgaggaccugaagauguaccagguggaguucaagaccaugaacgccaagcugcugauggaccccaagcggcagaucuuccuggaccagaacaugcuggccgugaucgacgagcugaugcaggcccugaacuucaacagcgagaccgugccccagaagagcagccuggaggagcccgacuucuacaagaccaagaucaagcugugcauccugcugcacgccuucagaaucagagccgugaccaucgacagagugaugagcuaccugaacgccagc

SEQ ID NO: 47 - protein, 25 aa, Homo sapiens
  misc_feature (1)..(25): Human CD80 Intracellular domain
TYCFAPRCRERRRNERLRRESVRPV

SEQ ID NO: 48 - protein, 550 aa, Homo sapiens
  misc_feature (1)..(550): Human PGFRB Intracellular domain (WT)
QKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 49 - protein, 22 aa, Homo sapiens
  misc_feature (1)..(22): Human PGFRB Intracellular domain (E570tr)
QKKPRYEIRWKVIESVSSDGHE

SEQ ID NO: 50 - protein, 183 aa, Homo sapiens
  misc_feature (1)..(183): Human PGFRB Intracellular domain (G739tr)
QKKPRYEIRWKVIESVSSDGHEFIFVDPMQLPYDSTWELPRDQLVLGRTLGSGAFGQVVEATAHGLSHSQATMKVAVAMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGG

SEQ ID NO: 51 - protein, 26 aa, Artificial Sequence
  Synthetic Linker
SGGGSGGGGSGGGGSGGGGSGGGSLQ

SEQ ID NO: 52 - RNA, 1791 nt, Artificial Sequence
  Synthetic hIL12AB-8TM Nucleotide Sequence
augugccaccagcagcuggugaucagcugguucagccugguguuccuggccagcccccugguggccaucugggagcugaagaaggacguguacgugguggaguuggauugguaccccgacgcccccggcgagaugguggugcugaccugcgacacccccgaggaggacggcaucaccuggacccuggaccagagcagcgaggugcugggcagcggcaagacccugaccauccaggugaaggaguucggcgacgccggccaguacaccugccacaagggcggcgaggugcugagccacagccugcugcugcugcacaagaaggaggacggcaucuggagcaccgacauccugaaggaccagaaggagcccaagaacaagaccuuccugagaugcgaggccaagaacuacagcggcagauucaccugcugguggcugaccaccaucagcaccgaccugaccuucagcgugaagagcagcagaggcagcagcgacccccagggcgugaccugcggcgccgccacccugagcgccgagagagugagaggcgacaacaaggaguacgaguacagcguggagugccaggaagauagcgccugccccgccgccgaggagagccugcccaucgaggugaugguggacgccgugcacaagcugaaguacgagaacuacaccagcagcuucuucaucagagauaucaucaagcccgacccccccaagaaccugcagcugaagccccugaagaacagccggcagguggaggugagcugggaguaccccgacaccuggagcaccccccacagcuacuucagccugaccuucugcgugcaggugcagggcaagagcaagagagagaagaaagauagaguguucaccgacaagaccagcgccaccgugaucugcagaaagaacgccagcaucagcgugagagcccaagauagauacuacagcagcagcuggagcgagugggccagcgugcccugcagcggcggcggcggcggcggcagcagaaaccugcccguggccacccccgaccccggcauguuccccugccugcaccacagccagaaccugcugagagccgugagcaacaugcugcagaaggcccggcagacccuggaguucuaccccugcaccagcgaggagaucgaccacgaagauaucaccaaagauaagaccagcaccguggaggccugccugccccuggagcugaccaagaacgagagcugccugaacagcagagagaccagcuucaucaccaacggcagcugccuggccagcagaaagaccagcuucaugauggcccugugccugagcagcaucuacgaggaccugaagauguaccagguggaguucaagaccaugaacgccaagcugcugauggaccccaagcggcagaucuuccuggaccagaacaugcuggccgugaucgacgagcugaugcaggcccugaacuucaacagcgagaccgugccccagaagagcagccuggaggagcccgacuucuacaagaccaagaucaagcugugcauccugcugcacgccuucagaaucagagccgugaccaucgacagagugaugagcuaccugaacgccagcucugguggcggaucaggcggcggcgguucaggaggcgguggaaguggagguggcgggucuggcggagguucacugcagaucuacaucugggcuccacuggccggcaccugcggcgugcugcugcugagccuggugaucacccuguacugcuacgggaaaccaauuccaaauccccuccugggguuggauagcacc

SEQ ID NO: 53 - protein, 597 aa, Artificial Sequence
  Synthetic hIL12AB-8TM Amino Acid Sequence
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGGSGGGGSGGGGSGGGGSGGGSLQIYIWAPLAGTCGVLLLSLVITLYCYGKPIPNPLLGLDST

SEQ ID NO: 54 - RNA, 1749 nt, Artificial Sequence
  Synthetic hIL12AB-8TM no epitope tag Nucleotide Sequence
augugccaccagcagcuggugaucagcugguucagccugguguuccuggccagcccccugguggccaucugggagcugaagaaggacguguacgugguggaguuggauugguaccccgacgcccccggcgagaugguggugcugaccugcgacacccccgaggaggacggcaucaccuggacccuggaccagagcagcgaggugcugggcagcggcaagacccugaccauccaggugaaggaguucggcgacgccggccaguacaccugccacaagggcggcgaggugcugagccacagccugcugcugcugcacaagaaggaggacggcaucuggagcaccgacauccugaaggaccagaaggagcccaagaacaagaccuuccugagaugcgaggccaagaacuacagcggcagauucaccugcugguggcugaccaccaucagcaccgaccugaccuucagcgugaagagcagcagaggcagcagcgacccccagggcgugaccugcggcgccgccacccugagcgccgagagagugagaggcgacaacaaggaguacgaguacagcguggagugccaggaagauagcgccugccccgccgccgaggagagccugcccaucgaggugaugguggacgccgugcacaagcugaaguacgagaacuacaccagcagcuucuucaucagagauaucaucaagcccgacccccccaagaaccugcagcugaagccccugaagaacagccggcagguggaggugagcugggaguaccccgacaccuggagcaccccccacagcuacuucagccugaccuucugcgugcaggugcagggcaagagcaagagagagaagaaagauagaguguucaccgacaagaccagcgccaccgugaucugcagaaagaacgccagcaucagcgugagagcccaagauagauacuacagcagcagcuggagcgagugggccagcgugcccugcagcggcggcggcggcggcggcagcagaaaccugcccguggccacccccgaccccggcauguuccccugccugcaccacagccagaaccugcugagagccgugagcaacaugcugcagaaggcccggcagacccuggaguucuaccccugcaccagcgaggagaucgaccacgaagauaucaccaaagauaagaccagcaccguggaggccugccugccccuggagcugaccaagaacgagagcugccugaacagcagagagaccagcuucaucaccaacggcagcugccuggccagcagaaagaccagcuucaugauggcccugugccugagcagcaucuacgaggaccugaagauguaccagguggaguucaagaccaugaacgccaagcugcugauggaccccaagcggcagaucuuccuggaccagaacaugcuggccgugaucgacgagcugaugcaggcccugaacuucaacagcgagaccgugccccagaagagcagccuggaggagcccgacuucuacaagaccaagaucaagcugugcauccugcugcacgccuucagaaucagagccgugaccaucgacagagugaugagcuaccugaacgccagcucugguggcggaucaggcggcggcgguucaggaggcgguggaaguggagguggcgggucuggcggagguucacugcagaucuacaucugggcuccacuggccggcaccugcggcgugcugcugcugagccuggugaucacccuguacugcuac

SEQ ID NO: 55 - protein, 583 aa, Artificial Sequence
  Synthetic hIL12AB-8TM no epitope tag Amino Acid Sequence
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGGSGGGGSGGGGSGGGGSGGGSLQIYIWAPLAGTCGVLLLSLVITLYCY

SEQ ID NO: 56 - RNA, 1812 nt, Artificial Sequence
  Synthetic h12AB-80TID Nucleotide Sequence 1 matched
augugccaccagcagcuggugaucagcugguucagccugguguuccuggccagcccccugguggccaucugggagcugaagaaggacguguacgugguggaguuggauugguaccccgacgcccccggcgagaugguggugcugaccugcgacacccccgaggaggacggcaucaccuggacccuggaccagagcagcgaggugcugggcagcggcaagacccugaccauccaggugaaggaguucggcgacgccggccaguacaccugccacaagggcggcgaggugcugagccacagccugcugcugcugcacaagaaggaggacggcaucuggagcaccgacauccugaaggaccagaaggagcccaagaacaagaccuuccugagaugcgaggccaagaacuacagcggcagauucaccugcugguggcugaccaccaucagcaccgaccugaccuucagcgugaagagcagcagaggcagcagcgacccccagggcgugaccugcggcgccgccacccugagcgccgagagagugagaggcgacaacaaggaguacgaguacagcguggagugccaggaagauagcgccugccccgccgccgaggagagccugcccaucgaggugaugguggacgccgugcacaagcugaaguacgagaacuacaccagcagcuucuucaucagagauaucaucaagcccgacccccccaagaaccugcagcugaagccccugaagaacagccggcagguggaggugagcugggaguaccccgacaccuggagcaccccccacagcuacuucagccugaccuucugcgugcaggugcagggcaagagcaagagagagaagaaagauagaguguucaccgacaagaccagcgccaccgugaucugcagaaagaacgccagcaucagcgugagagcccaagauagauacuacagcagcagcuggagcgagugggccagcgugcccugcagcggcggcggcggcggcggcagcagaaaccugcccguggccacccccgaccccggcauguuccccugccugcaccacagccagaaccugcugagagccgugagcaacaugcugcagaaggcccggcagacccuggaguucuaccccugcaccagcgaggagaucgaccacgaagauaucaccaaagauaagaccagcaccguggaggccugccugccccuggagcugaccaagaacgagagcugccugaacagcagagagaccagcuucaucaccaacggcagcugccuggccagcagaaagaccagcuucaugauggcccugugccugagcagcaucuacgaggaccugaagauguaccagguggaguucaagaccaugaacgccaagcugcugauggaccccaagcggcagaucuuccuggaccagaacaugcuggccgugaucgacgagcugaugcaggcccugaacuucaacagcgagaccgugccccagaagagcagccuggaggagcccgacuucuacaagaccaagaucaagcugugcauccugcugcacgccuucagaaucagagccgugaccaucgacagagugaugagcuaccugaacgccagcucugguggcggaucaggcggcggcgguucaggaggcgguggaaguggagguggcgggucuggcggagguucacugcagcugcugcccagcugggccaucacccugaucagcgugaacggcaucuucgugaucugcugccugaccuacugcuucgccccucgaugcagagagagaagaagaaacgagagacugagaagagagagcgugcgacccgug

SEQ ID NO: 57 - RNA, 1812 nt, Artificial Sequence
  Synthetic h12AB-80TID Nucleotide Sequence 2 IL12_041
augugccaccagcagcuggugaucagcugguucagccugguguuccuggccagcccucugguggccaucugggagcugaagaaggacguguacgugguggagcuggacugguacccugacgccccuggcgagaugguggugcugaccugcgacaccccugaggaggacggcaucaccuggacccuggaccagagcagcgaggugcugggcagcggcaagacccugaccauccaggugaaggaguucggcgacgccggccaguacaccugccacaagggcggagagguguuaagccacagccugcucuugcuacacaagaaggaggacgguauuuggagcaccgacauccugaaggaccagaaggagccuaagaacaagaccuuccugaggugcgaggccaagaacuacagcggcagauucaccugcugguggcugaccaccaucuccacggaccugaccuucagcguuaagaguagcagaggcagcagcgacccucagggcgugacuuguggcgccgccacccugagcgccgagagagugagaggcgacaacaaggaguacgaguacagcguggagugccaggaggacagcgccugcccugccgccgaggagagccugccuaucgaggugaugguggacgccgugcacaagcugaaguacgagaauuacaccucauccuucuucaucagagacaucaucaagccugacccuccaaagaaucugcagcugaagccucugaagaacagcagacagguggaggugagcugggaguauccggauaccuggagcacaccucacagcuacuucucacuuacauucugcgugcaggugcagggcaagagcaagagagagaagaaggauagaguguucaccgacaagaccagcgccaccgugaucugcagaaagaacgccagcaucucagugagagcccaggacagauacuacucauccuccuggagcgagugggccagcgugccuugcuccgguggugguggcggaggcagcagaaaccugccuguggcuacaccugauccuggcauguucccuugccugcaccacagccagaaccugcugagagccgugagcaacaugcugcagaaggccagacagacucuggaguucuacccuugcaccagcgaggagaucgaccacgaggacaucaccaaggauaagacaagcaccguggaggccugccugccucuggagcugaccaagaacgagagcugccuaaacucuagggaaaccagcuucauuacuaacggcaguugcuuagccagccggaagacaucguucaugauggcccugugccugagcagcaucuacgaggaccugaagauguaccaaguggaguucaagaccaugaacgccaagcugcugauggacccuaagagacagaucuuccuagaccagaacaugcucgccgugaucgacgagcugaugcaggcccugaacuucaacagcgaaacugugccucagaagaguucacuggaggagccugacuucuauaagacuaagaucaagcuguguauucuccuccacgccuucagaaucagggcugucaccaucgauagggugaugagcuaccugaacgcaucguccggcggaggauccggaggaggaggcuccggcggugguggaaguggaggagguggaucaggaggcgguagucuccagcuccugccuagcugggccaucacccugaucucuguaaacggcauuuucgucauuugcugucugacuuacugcuucgccccuaggugccgggagcguaggagaaacgagagacugcgccgggaguccgugcggccugug

SEQ ID NO: 58 - RNA, 1812 nt, Artificial Sequence
  Synthetic h12AB-80TID Nucleotide Sequence 3 IL12_042
augugucaccagcagcucgugaucagcugguucagccugguguuccuggccuccccgcugguggccaucugggagcugaagaaggacguguacgugguggagcuggacugguaccccgacgcucccggcgagaugguggugcugaccugcgacacaccggaggaagacggaaucaccuggacccuggaccaauccuccgaaguucugggcagcggcaagacccugaccauccaggugaaggaguucggcgacgccggccaguacaccugccacaagggcggcgaggugcugagccacagccugcuccuccuccacaagaaagaggacggcaucuggagcaccgacauccugaaggaccagaaggagcccaagaacaagaccuuccugcggugcgaggccaagaacuacucaggccgauucaccuguugguggcucacaacuaucagcacagaccugaccuucagcgugaagucuagccggggcagcagcgauccucagggcgugaccugcggcgccgccacccugagcgccgagcgggugcggggcgacaacaaggaguacgaguacagcguggagugccaggaggacagcgccugcccggccgccgaagagucccugcccaucgaggugaugguggacgccgugcacaaacucaaguacgagaacuacaccuccagcuucuucauccgggacaucaucaagcccgauccgcccaagaaccugcagcugaagccccugaagaacagccggcagguggaggugagcugggaauacccggacacgugguccaccccacacagcuacuucagccugaccuuuugcgugcagguccaaggcaagagcaagcgggagaagaaggaccggguguucaccgauaagaccucagccaccgugauuugcagaaagaacgcauccauauccguacgcgcccaggaucgguacuacagcagcagcuggagcgagugggccagcgugcccuguagcggcggcggcgguggugggagucgcaaccugcccguggccaccccggaccccggcauguuccccugccugcaccacagccagaaccugcugcgggccgugagcaacaugcugcagaaggcccggcagacccuggaguucuaccccugcaccagcgaggagaucgaccacgaggacaucaccaaggacaagaccagcaccguggaggccugccugccccuggagcugaccaagaacgagagcugccugaacagucgcgaaaccuccuucauuacgaacggcagcugccuggccagccggaagaccagcuucaugauggcccugugccugagcagcaucuacgaggaccugaagauguaccagguggaguucaagaccaugaacgccaagcugcugauggaccccaagcggcagaucuuccuggaccagaacaugcuggccgugaucgacgagcugaugcaggcccugaacuucaacagcgaaaccgugccccagaaguccagccuggaggagcccgacuucuacaagaccaagaucaagcugugcauccugcugcacgccuuccggauccgggccgugaccaucgaccgggugaugagcuaccugaacgccucuuccgguggcgggagcggaggcgguggaucuggcggaggagggucgggaggcggcggaagcggugguggaagccuucaacugcugcccucgugggccaucacacugaucuccgugaacggcaucuucgugaucugcugccugaccuacugcuucgccccucggugccgcgagcgacggagaaacgagaggcucagacgggagagcgugcggcccgug

SEQ ID NO: 59 - RNA, 1812 nt, Artificial Sequence
  Synthetic h12AB-80TID Nucleotide Sequence 4 IL12_043
augugccaccaacagcuggugaucagcugguucagccugguguuccuggccucaccccugguggccaucugggagcugaagaaggacguguacgugguggagcuggacugguaccccgacgccccuggcgagaugguggugcugaccugcgacacgcccgaggaagacgguaucaccuggacucuggaccagagcagcgaggugcugggcagcggcaagacccugaccauccaggugaaggaguucggcgacgccggccaguacaccugccacaagggcggcgaagugcugagccacagccuucugcugcugcacaagaaggaggacggcauuuggagcaccgacauccugaaggaccagaaggagcccaagaacaagaccuuccugcggugcgaggccaagaacuacagcggccgguucaccugcugguggcugaccaccaucagcaccgaccuuaccuucagcguuaagagcagccggggcagcagcgauccccagggcgugaccugcggagccgccacccucuccgcagagcgggugcguggcgacaacaaggaguacgaguacagcguggagugccaggaggauagcgccugucccgcugccgaagagagccugcccaucgaggugaugguggacgccgugcacaagcugaaguacgagaacuacaccagcagcuucuucauccgggacaucaucaagcccgauccacccaagaaccugcagcugaagccccugaagaacagcaggcagguugaggugagcugggaauaccccgacaccuggagcaccccucacagcuacuucagccugaccuucugcgugcaggugcagggcaagagcaagcgggagaagaaggaucggguguucaccgauaagaccagcgccaccgugaucugccggaagaacgccagcaucagcguucgggcccaggaccgguacuacagcagcagcuggagcgagugggccagcgugcccugcucuggaggcggaggcggaggcucacggaaccugccaguggccacgccggaucccggcauguuccccugccugcaccacagccagaaccugcugcgggccgugagcaacaugcugcagaaggcccggcagacccuggaguucuaccccugcaccagcgaggagaucgaccacgaggacaucaccaaggacaagaccagcaccguggaggccugccugccccuggagcugaccaagaacgagagcugccugaacagccgggagacaagcuucaucaccaacggcagcugccuggccagccggaagaccagcuucaugauggcccugugccugagcagcaucuacgaggaccugaagauguaccagguggaguucaagaccaugaacgccaagcugcugauggaccccaagcggcagaucuuccuggaucagaacaugcuggccgugaucgacgagcugaugcaggcccugaacuucaacagcgagacugugccccagaaguccagccuggaggagcccgacuucuacaagaccaagaucaagcugugcauccugcugcacgccuuccggauccgggcugugaccaucgaccgggugaugagcuaccugaacgccucuuccggcggcggaucgggagguggagguucuggaggagguggaagcggugguggcggaagcggcgguggcagccugcaauugcuccccagcugggccaucacccugaucagcgugaacggcaucuucgugaucuguugccugaccuacugcuucgccccacggugccgggagagacggcggaacgagcggcugcggcgagagagcgugcggcccgug

SEQ ID NO: 60 - RNA, 1812 nt, Artificial Sequence
  Synthetic h12AB-80TID Nucleotide Sequence 5 IL12_044
augugucaccagcaguuggugauaucuugguucucacugguguuucuugcaucaccacucguggcgaucugggaacuuaagaaggacgucuacgugguggaguuagauugguauccugacgcacccggggaaaugguuguccucacgugcgacacuccagaggaagacgggaucaccuggacccuggaucagucgucagagguacuuggcaguggcaagacacugacaauccagguuaaagaguuuggugacgccgggcaguauacgugccacaagggcggcgagguguugucacauucucugcuucuccugcacaagaaagaagacggcaucuggucaacugacauccugaaagaccagaaagaacccaagaauaagaccuuccuccguugcgaagcaaagaacuacucagggcguuucacuugcugguggcuaaccacaauuucuaccgaucugacguucucugugaagucaaguaggggauccucagacccucaaggggucaccugcggcgccgccaccuuauccgccgaaagaguucggggugacaauaaagaguacgaguacagcgucgagugucaggaggacuccgccuguccugcugcagaggagucccugccgaucgaaguuaugguggacgccguccacaagcucaaauacgagaacuacaccucaagcuucuucaucagagacaucaucaagccugauccacccaagaaccugcaacugaagccuuugaagaacagccgacagguggagguuucuugggaauauccagacacguggaguacgccccauuccuacuucagcuugaccuucugcgugcagguucaggggaaguccaagagagagaagaaggaucguguguucacagacaagaccuccgccaccgugaucugccggaagaacgcaucuaucaguguuagggcccaggaucgguacuacucgaguuccuggucugagugggcaagugugcccugcuccgguggcggcggaggagggucaaggaaccugcccguugccacaccagauccaggaauguuccccugucugcaccacucucagaaccuuuugcgagccguuucuaauaugcuucagaaggcucggcagacccuugaguuuuaucccugcacgucugaggagaucgaucacgaggacaucaccaaggacaagacuuccaccguugaagccuguuuaccucuggaacugaccaagaacgaauccugucucaacaguagggaaacgagcuucaucacuaacggaagcugucuggcuagccggaagaccucuuuuaugauggcccugugcuugagcucuauuuacgaagauuugaagauguaccaaguggaauuuaagacuaugaacgccaaacugcugauggacccuaagcgccaaaucuucuuggaucagaauaugcuggcuguaaucgacgagcucaugcaggcucugaacuucaacagcgagacgguaccgcagaagaguucccuggaagaaccggacuucuacaagacuaagauuaaacucugcauacuccuccacgccuuccggaucagggccgucacaauagauagggucaugaguuaucuuaacgcgaguucuggugguggaucggguggcggaggcucaggaggaggcgguucuggcgguggugggaguggaggcgguagucugcagcugcugccgaguugggcaaucacgcuaaucagcgugaacggaauauucguaauuuguugccucaccuauuguuucgcacccaggugcagggaaaggaggcgaaacgaaagguugaggagggaaucuguccggccagug

SEQ ID NO: 61 - protein, 604 aa, Homo sapiens
  misc_feature (1)..(604): h12AB-80TID Amino Acid Sequence  1
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGGSGGGGSGGGGSGGGGSGGGSLQLLPSWAITLISVNGIFVICCLTYCFAPRCRERRRNERLRRESVRPV

SEQ ID NO: 62 - RNA, 75 nt, Homo sapiens
  misc_feature (1)..(75): Human PGFRB transmembrane domain nucleotide sequence
gugguggugaucagcgccauccuggcccugguggugcugaccaucaucagccugaucauccugaucaugcugugg

SEQ ID NO: 63 - RNA, 66 nt, Homo sapiens
  misc_feature (1)..(66): Human PGFRB E570tr intracellular domain nucleotide sequence
cagaagaagcccagauacgagaucagauggaaggugaucgagagcgugagcagcgacggccacgag

SEQ ID NO: 64 - RNA, 549 nt, Homo sapiens
  misc_feature (1)..(549): Human PGFRB G739tr intracellular domain nucleotide sequence
cagaagaagcccagauacgagauccgguggaaggugaucgagagcgugagcagcgacggccacgaguucaucuucguggaccccaugcagcugcccuacgacagcaccugggagcugccccgugaucagcuggugcugggcagaacccugggcagcggcgccuucggccaggugguggaggcuaccgcccacggccugagccacagccaggccaccaugaagguggccguggccaugcucaagagcaccgccagaagcagcgagaagcaggcccugaugagcgagcugaagaucaugagccaucuggggccccaccugaacguggugaaccugcugggcgccugcaccaagggcggccccaucuacaucaucaccgaguacugcagauacggcgaccugguggacuaccugcacagaaacaagcacaccuuccugcagcaccacagcgacaagagaagaccucccagcgccgagcuguacagcaacgcccugcccguuggucugccccuacccagccacgugagccugaccggcgagagcgacggcggc

SEQ ID NO: 65 - RNA, 1815 nt, Artificial Sequence
  Synthetic h12AB-PTM-ICD-E570tr Nucleotide Sequence
augugccaccagcagcuggugaucagcugguucagccugguguuccuggccagcccccugguggccaucugggagcugaagaaggacguguacgugguggaguuggauugguaccccgacgcccccggcgagaugguggugcugaccugcgacacccccgaggaggacggcaucaccuggacccuggaccagagcagcgaggugcugggcagcggcaagacccugaccauccaggugaaggaguucggcgacgccggccaguacaccugccacaagggcggcgaggugcugagccacagccugcugcugcugcacaagaaggaggacggcaucuggagcaccgacauccugaaggaccagaaggagcccaagaacaagaccuuccugagaugcgaggccaagaacuacagcggcagauucaccugcugguggcugaccaccaucagcaccgaccugaccuucagcgugaagagcagcagaggcagcagcgacccccagggcgugaccugcggcgccgccacccugagcgccgagagagugagaggcgacaacaaggaguacgaguacagcguggagugccaggaagauagcgccugccccgccgccgaggagagccugcccaucgaggugaugguggacgccgugcacaagcugaaguacgagaacuacaccagcagcuucuucaucagagauaucaucaagcccgacccccccaagaaccugcagcugaagccccugaagaacagccggcagguggaggugagcugggaguaccccgacaccuggagcaccccccacagcuacuucagccugaccuucugcgugcaggugcagggcaagagcaagagagagaagaaagauagaguguucaccgacaagaccagcgccaccgugaucugcagaaagaacgccagcaucagcgugagagcccaagauagauacuacagcagcagcuggagcgagugggccagcgugcccugcagcggcggcggcggcggcggcagcagaaaccugcccguggccacccccgaccccggcauguuccccugccugcaccacagccagaaccugcugagagccgugagcaacaugcugcagaaggcccggcagacccuggaguucuaccccugcaccagcgaggagaucgaccacgaagauaucaccaaagauaagaccagcaccguggaggccugccugccccuggagcugaccaagaacgagagcugccugaacagcagagagaccagcuucaucaccaacggcagcugccuggccagcagaaagaccagcuucaugauggcccugugccugagcagcaucuacgaggaccugaagauguaccagguggaguucaagaccaugaacgccaagcugcugauggaccccaagcggcagaucuuccuggaccagaacaugcuggccgugaucgacgagcugaugcaggcccugaacuucaacagcgagaccgugccccagaagagcagccuggaggagcccgacuucuacaagaccaagaucaagcugugcauccugcugcacgccuucagaaucagagccgugaccaucgacagagugaugagcuaccugaacgccagcucugguggcggaucaggggguggcggaucuggcggggguggaaguggagguggcgggucuggcggagguucacugcaggugguggugaucagcgccauccuggcccugguggugcugaccaucaucagccugaucauccugaucaugcuguggcagaagaagcccagauacgagaucagauggaaggugaucgagagcgugagcagcgacggccacgag

SEQ ID NO: 66 - protein, 605 aa, Artificial Sequence
  Synthetic h12AB-PTM-ICD-E570tr Amino Acid Sequence
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGGSGGGGSGGGGSGGGGSGGGSLQVVVISAILALVVLTIISLIILIMLWQKKPRYEIRWKVIESVSSDGHE

SEQ ID NO: 67 - RNA, 2298 nt, Artificial Sequence
  Synthetic h12AB-PTM-ICD-G739tr Nucleotide Sequence
augugccaccagcagcuggugaucagcugguucagccugguguuccuggccagcccccugguggccaucugggagcugaagaaggacguguacgugguggaguuggauugguaccccgacgcccccggcgagaugguggugcugaccugcgacacccccgaggaggacggcaucaccuggacccuggaccagagcagcgaggugcugggcagcggcaagacccugaccauccaggugaaggaguucggcgacgccggccaguacaccugccacaagggcggcgaggugcugagccacagccugcugcugcugcacaagaaggaggacggcaucuggagcaccgacauccugaaggaccagaaggagcccaagaacaagaccuuccugagaugcgaggccaagaacuacagcggcagauucaccugcugguggcugaccaccaucagcaccgaccugaccuucagcgugaagagcagcagaggcagcagcgacccccagggcgugaccugcggcgccgccacccugagcgccgagagagugagaggcgacaacaaggaguacgaguacagcguggagugccaggaagauagcgccugccccgccgccgaggagagccugcccaucgaggugaugguggacgccgugcacaagcugaaguacgagaacuacaccagcagcuucuucaucagagauaucaucaagcccgacccccccaagaaccugcagcugaagccccugaagaacagccggcagguggaggugagcugggaguaccccgacaccuggagcaccccccacagcuacuucagccugaccuucugcgugcaggugcagggcaagagcaagagagagaagaaagauagaguguucaccgacaagaccagcgccaccgugaucugcagaaagaacgccagcaucagcgugagagcccaagauagauacuacagcagcagcuggagcgagugggccagcgugcccugcagcggcggcggcggcggcggcagcagaaaccugcccguggccacccccgaccccggcauguuccccugccugcaccacagccagaaccugcugagagccgugagcaacaugcugcagaaggcccggcagacccuggaguucuaccccugcaccagcgaggagaucgaccacgaagauaucaccaaagauaagaccagcaccguggaggccugccugccccuggagcugaccaagaacgagagcugccugaacagcagagagaccagcuucaucaccaacggcagcugccuggccagcagaaagaccagcuucaugauggcccugugccugagcagcaucuacgaggaccugaagauguaccagguggaguucaagaccaugaacgccaagcugcugauggaccccaagcggcagaucuuccuggaccagaacaugcuggccgugaucgacgagcugaugcaggcccugaacuucaacagcgagaccgugccccagaagagcagccuggaggagcccgacuucuacaagaccaagaucaagcugugcauccugcugcacgccuucagaaucagagccgugaccaucgacagagugaugagcuaccugaacgccagcucugguggcggaucaggcggcggcgguucaggaggcgguggaaguggagguggcgggucuggcggagguucacugcaggugguggugaucagcgccauccuggcccugguggugcucaccaucaucagccugaucauccugaucaugcuguggcagaagaagcccagauacgagauccgguggaaggugaucgagagcgugagcagcgacggccacgaguucaucuucguggaccccaugcagcugcccuacgacagcaccugggagcugccccgugaucagcuggugcugggcagaacccugggcagcggcgccuucggccaggugguggaggcuaccgcccacggccugagccacagccaggccaccaugaagguggccguggccaugcucaagagcaccgccagaagcagcgagaagcaggcccugaugagcgagcugaagaucaugagccaucuggggccccaccugaacguggugaaccugcugggcgccugcaccaagggcggccccaucuacaucaucaccgaguacugcagauacggcgaccugguggacuaccugcacagaaacaagcacaccuuccugcagcaccacagcgacaagagaagaccucccagcgccgagcuguacagcaacgcccugcccguuggucugccccuacccagccacgugagccugaccggcgagagcgacggcggc

SEQ ID NO: 68 - protein, 766 aa, Artificial Sequence
  Synthetic h12AB-PTM-ICD-G739tr Amino Acid Sequence
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGGSGGGGSGGGGSGGGGSGGGSLQVVVISAILALVVLTIISLIILIMLWQKKPRYEIRWKVIESVSSDGHEFIFVDPMQLPYDSTWELPRDQLVLGRTLGSGAFGQVVEATAHGLSHSQATMKVAVAMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGG

SEQ ID NO: 69 - RNA, 75 nt, Homo sapiens
  misc_feature (1)..(75): Human CD8 transmembrane nucleotide sequence
aucuacaucugggcuccacuggccggcaccugcggcgugcugcugcugagccuggugaucacccuguacugcuac

SEQ ID NO: 70 - RNA, 63 nt, Homo sapiens
  misc_feature (1)..(63): Human CD80 transmembrane domain nucleotide sequence
cugcugcccagcugggccaucacccugaucagcgugaacggcaucuucgugaucugcugccug

SEQ ID NO: 71 - RNA, 75 nt, Homo sapiens
  misc_feature (1)..(75): Human CD80 intracellular domain nucleotide sequence
accuacugcuucgccccucgaugcagagagagaagaagaaacgagagacugagaagagagagcgugcgacccgug

SEQ ID NO: 72 - RNA, 85 nt, Artificial Sequence
  Synthetic miR-122
ccuuagcagagcuguggagugugacaaugguguuugugucuaaacuaucaaacgccauuaucacacuaaauagcuacugcuaggc

SEQ ID NO: 73 - RNA, 22 nt, Artificial Sequence
  Synthetic miR-122-3p
aacgccauuaucacacuaaaua

SEQ ID NO: 74 - RNA, 22 nt, Artificial Sequence
  Synthetic miR-122-3p binding site
uauuuagugugauaauggcguu

SEQ ID NO: 75 - RNA, 92 nt, Artificial Sequence
  Synthetic 5 UTR
ucaagcuuuuggacccucguacagaagcuaauacgacucacuauagggaaauaagagagaaaagaagaguaagaagaaauauaagagccacc

SEQ ID NO: 76 - RNA, 47 nt, Artificial Sequence
  Synthetic 5 UTR
gggaaauaagagagaaaagaagaguaagaagaaauauaagagccacc

SEQ ID NO: 77 - RNA, 141 nt, Artificial Sequence
  Synthetic 3′ UTR
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 78 - RNA, 164 nt, Artificial Sequence
  Synthetic 3 UTR with mi-122 and mi-142.3p sites
ugauaauaggcuggagccucgguggccaugcuucuugccccuugggcccaaacaccauugucacacuccauccccccagccccuccuccccuuccuccauaaaguaggaaacacuacaugcacccguacccccguggucuuugaauaaagucugagugggcggc

SEQ ID NO: 79 - protein, 22 aa, Artificial Sequence
  Synthetic 2A peptide amino acid sequence
GSGATNFSLLKQAGDVEENPGP

SEQ ID NO: 80 - DNA, 66 bp, Artificial Sequence
  Synthetic Nucleotide sequence encoding 2A peptide
ggaagcggagctactaacttcagcctgctgaagcaggctggagacgtggaggagaaccctggacct

SEQ ID NO: 81 - DNA, 108 bp, Artificial Sequence
  Synthetic Nucleotide sequence encoding 2A peptide
tccggactcagatccggggatctcaaaattgtcgctcctgtcaaacaaactcttaactttgatttactcaaactggctggggatgtagaaagcaatccaggtccactc

SEQ ID NO: 82 - RNA, 22 nt, Artificial Sequence
  Synthetic miR-122-5p
uggagugugacaaugguguuug

SEQ ID NO: 83 - RNA, 22 nt, Artificial Sequence
  Synthetic miR-122-5p binding site
caaacaccauugucacacucca

SEQ ID NO: 84 - RNA, 22 nt, Artificial Sequence
  Synthetic has-miR-21-5p
uagcuuaucagacugauguuga

SEQ ID NO: 85 - RNA, 21 nt, Artificial Sequence
  Synthetic has-miR-21-3p
caacaccagucgaugggcugu

SEQ ID NO: 86 - protein, 25 aa, Artificial Sequence
  Synthetic Linker
  misc_feature (6)..(25): “GGGGS” may or may not be present
GGGGSGGGGSGGGGSGGGGSGGGGS

SEQ ID NO: 87 - protein, 10 aa, Artificial Sequence
  Synthetic Linker
GGSGGGGSGG

SEQ ID NO: 88 - protein, 8 aa, Artificial Sequence
  Synthetic Linker
GGSGGGGG

SEQ ID NO: 89 - protein, 8 aa, Artificial Sequence
  Synthetic Linker
GSGSGSGS

SEQ ID NO: 90 - protein, 7 aa, Artificial Sequence
  Synthetic Linker
GGGGGGS

SEQ ID NO: 91 - protein, 5 aa, Artificial Sequence
  Synthetic Linker
GGGGG

SEQ ID NO: 92 - protein, 6 aa, Artificial Sequence
  Synthetic Linker
GGGGGG

SEQ ID NO: 93 - protein, 7 aa, Artificial Sequence
  Synthetic Linker
GGGGGGG

SEQ ID NO: 94 - protein, 8 aa, Artificial Sequence
  Synthetic Linker
GGGGGGGG

SEQ ID NO: 95 - protein, 25 aa, Artificial Sequence
  Synthetic Linker
  misc_feature (6)..(25): “EAAAK” may or may not be present
EAAAKEAAAKEAAAKEAAAKEAAAK

SEQ ID NO: 96 - protein, 15 aa, Artificial Sequence
  Synthetic Linker
EAAAKEAAAKEAAAK

SEQ ID NO: 97 - protein, 16 aa, Artificial Sequence
  Synthetic Linker
GGGGSLVPRGSGGGGS

SEQ ID NO: 98 - protein, 6 aa, Artificial Sequence
  Synthetic Linker
GSGSGS

SEQ ID NO: 99 - protein, 15 aa, Artificial Sequence
  Synthetic Linker
GGGGSLVPRGSGGGG

SEQ ID NO: 100 - protein, 11 aa, Artificial Sequence
  Synthetic Linker
GGSGGHMGSGG

SEQ ID NO: 101 - protein, 11 aa, Artificial Sequence
  Synthetic Linker
GGSGGSGGSGG

SEQ ID NO: 102 - protein, 5 aa, Artificial Sequence
  Synthetic Linker
GGSGG

SEQ ID NO: 103 - protein, 8 aa, Artificial Sequence
  Synthetic Linker
GSGSGSGS

SEQ ID NO: 104 - protein, 18 aa, Artificial Sequence
  Synthetic Linker
GGGSEGGGSEGGGSEGGG

SEQ ID NO: 105 - protein, 8 aa, Artificial Sequence
  Synthetic Linker
AAGAATAA

SEQ ID NO: 106 - protein, 5 aa, Artificial Sequence
  Synthetic Linker
GGSSG

SEQ ID NO: 107 - protein, 11 aa, Artificial Sequence
  Synthetic Linker
GSGGGTGGGSG

SEQ ID NO: 108 - protein, 12 aa, Artificial Sequence
  Synthetic Linker
GSGSGSGSGGSG

SEQ ID NO: 109 - protein, 14 aa, Artificial Sequence
  Synthetic Linker
GSGGSGSGGSGGSG

SEQ ID NO: 110 - protein, 14 aa, Artificial Sequence
  Synthetic Linker
GSGGSGGSGGSGGS

SEQ ID NO: 111 - RNA, 12 nt, Artificial Sequence
  Synthetic GC-motif, [CCG]4
ccgccgccgccg

SEQ ID NO: 112 - RNA, 15 nt, Artificial Sequence
  Synthetic GC-motif, [CCG]5
ccgccgccgccgccg

SEQ ID NO: 113 - RNA, 10 nt, Artificial Sequence
  Synthetic V1 GC-rich RNA element
ccccggcgcc

SEQ ID NO: 114 - RNA, 7 nt, Artificial Sequence
  Synthetic V2 GC-rich RNA element
ccccggc

SEQ ID NO: 115 - RNA, 6 nt, Artificial Sequence
  Synthetic EK GC-rich RNA element
gccgcc

SEQ ID NO: 116 - DNA, 41 bp, Artificial Sequence
  Synthetic 5UTR
gggaaataagagagaaaagaagagtaagaagaaatataaga

SEQ ID NO: 117 - DNA, 57 bp, Artificial Sequence
  Synthetic V1-5UTR
gggaaataagagagaaaagaagagtaagaagaaatataagaccccggcgccgccacc

SEQ ID NO: 118 - DNA, 54 bp, Artificial Sequence
  Synthetic V2-5UTR
gggaaataagagagaaaagaagagtaagaagaaatataagaccccggcgccacc

SEQ ID NO: 119 - DNA, 47 bp, Artificial Sequence
  Synthetic Standard UTR
gggaaataagagagaaaagaagagtaagaagaaatataagagccacc

SEQ ID NO: 120 - RNA, 6 nt, Artificial Sequence
  Synthetic Kozak Consensus
gccrcc

SEQ ID NO: 121 - RNA, 141 nt, Artificial Sequence
  Synthetic 3UTR with miR-122
ugauaauaggcuggagccucgguggccuagcuucuugccccuugggccuccccccagccccuccuccccuuccugcacccguaccccccaaacaccauugucacacuccaguggucuuugaauaaagucugagugggcggc

SEQ ID NO: 122 - RNA, 447 nt, Homo sapiens
  misc_feature (1)..(447): HS IL15opt-tPA6 mRNA ORF #2
auggacgcaaugaagagagggcucugcugugugcuccuucuuugcggagcagucuucguuucgcccagccaggaaauccacgcccgauucagaagaggagccagaaacugggugaacgugaucucggaccuuaagaagaucgaggaccucauccagucgaugcacaucgacgcgacgcuguacacggagucggacguccacccgucgugcaaggucacggcgaugaagugcuuccuccuggagcuccaagucaucucgcucgagagcggugacgcgucgauccacgacacgguggagaaccugaucauccuggcgaacaacucgcugucgucgaacgggaacgucacagaguccggcugcaaggagugcgaggagcuggaggagaagaacaucaaggaguuccugcagucguucguucauauaguccagauguucaucaacacgucg

SEQ ID NO: 123 - protein, 314 aa, Artificial Sequence
  Synthetic Hs tPA6-ILR_linker_CD80TID
MDAMKRGLCCVLLLCGAVFVSPSQEIHARFRRGARNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSGGGGSGGGGSGGGGSLLPSWAITLISVNGIFVICCLTYCFAPRCRERRRNERLRRESVRPV

SEQ ID NO: 124 - DNA, 942 bp, Artificial Sequence
  Synthetic Nucleotide Sequence #1 Hs tPA6-ILR_linker_CD80TID
atggatgcaatgaagagagggctctgctgtgtgctgctgctgtgtggagcagtcttcgtttcgcccagccaggaaatccatgcccgattcagaagaggagccagaaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggcgacgcgtcgatccacgacacggtggagaacctgatcatcctggcgaacaactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgtctggtggcggatcaggcggtggcggatctggcggcggtggaagtggaggtggcgggtctggcggaggttcactgcagatcacctgtccacctcccatgtccgtggaacacgccgacatctgggtcaagagctacagcctgtactcccgggaaagatacatttgtaacagcgggtttaagaggaaggccggcacctcctcgttgaccgaatgcgtgctgaacaaggctaccaacgtggcccattggactaccccgtccctgaagtgcattcgcgatcctgccctcgtgcaccaacggcctgcgccgccgtccggcggcggcggttcaggcggaggaggaagtggaggtggaggctccctgctgcccagctgggccatcaccctgatcagcgtgaacggcatcttcgtgatctgctgcctgacctactgcttcgctcccagatgcagagagagaagaagaaacgagagactgagaagagagagcgtgagacccgtg

SEQ ID NO: 125 - DNA, 942 bp, Artificial Sequence
  Synthetic Nucleotide Sequence #2 Hs tPA6-ILR_GS_CD80TID-partopt
atggatgcaatgaagagagggctctgctgtgtgctgctgctgtgtggagcagtcttcgtttcgcccagccaggaaatccatgcccgattcagaagaggagccagaaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggcgacgcgtcgatccacgacacggtggagaacctgatcatcctggcgaacaactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgagcggcggcggtagtggaggaggtggctccggtggaggcggcagcggtggcggtggaagtggcggcggcagcctgcagatcacctgtcctcctcccatgagcgtggagcacgccgacatctgggtgaagagctacagcctgtacagccgggagcggtacatctgcaacagcggcttcaagcggaaggccggcacaagcagcctgaccgagtgcgtgctgaacaaggccaccaacgtggcccactggaccaccccaagcctgaagtgcatccgggaccccgccctggtgcatcagcggcccgccccacctagcggcggaggaggctccggaggaggcgggagcggtggaggcggcagcctgctgccctcttgggccatcaccctgatcagcgtgaacggcatcttcgtgatctgctgcctgacctactgctttgcccctaggtgccgggagagacggcggaacgagaggctgcggcgggagagcgtgcggcccgtg

SEQ ID NO: 126 - DNA, 942 bp, Artificial Sequence
  Synthetic Nucleotide Sequence #3 Hs tPA6-ILR_GS_CD80TID_fullopt
atggacgccatgaagcggggcctgtgctgcgtgctgctgctgtgcggcgccgtgttcgtgagccccagccaggagatccacgcccggtttagacggggcgcacggaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctctggtggcggctccggtggaggtggctctggcggcggaggtagcggtggcggaggcagtggcggagggagcctgcagatcacctgcccaccacccatgagcgtggagcacgccgacatctgggtgaagagctacagcctgtacagccgggagcggtacatctgcaacagcggcttcaagcggaaggccggcaccagcagcctgaccgagtgcgtgctgaacaaggccaccaacgtggcccactggaccaccccgagcctgaagtgcatccgggacccagccctggtgcatcagcggcccgcacctccaagcggcggaggcggaagtggcggcggcggttcaggcggaggaggcagcctcctgccctcttgggccatcaccctgatcagcgtgaacggcatcttcgtgatctgctgcctgacctactgcttcgcaccccggtgcagagagcggcggcggaacgagcgcctgcggcgagagagcgtgcggcccgtg

SEQ ID NO: 127 - RNA, 23 nt, Artificial Sequence
  Synthetic hsa-miR-142-3p
uguaguguuuccuacuuuaugga

SEQ ID NO: 128 - RNA, 21 nt, Artificial Sequence
  Synthetic hsa-miR-142-5p
cauaaaguagaaagcacuacu

SEQ ID NO: 129 - protein, 78 aa, Artificial Sequence
  Synthetic IL-15Ra sushi domain
ITCPPPMSVEHADIWVKSYSLYSRERYICNSGFKRKAGTSSLTECVLNKATNVAHWTTPSLKCIRDPALVHQRPAPPS

SEQ ID NO: 130 - protein, 23 aa, Artificial Sequence
  Synthetic IL-15Ra transmembrane domain
VAISTSTVLLCGLSAVSLLACYL

SEQ ID NO: 131 - protein, 39 aa, Artificial Sequence
  Synthetic IL-15Ra intracellular domain
KSRQTPPLASVEMEAMEALPVTWGTSSRDEDLENCSHHL

SEQ ID NO: 132 - RNA, 41 nt, Artificial Sequence
  Synthetic 5UTR
gggaaauaagagagaaaagaagaguaagaagaaauauaaga

SEQ ID NO: 133 - RNA, 57 nt, Artificial Sequence
  Synthetic V1-5UTR
gggaaauaagagagaaaagaagaguaagaagaaauauaagaccccggcgccgccacc

SEQ ID NO: 134 - RNA, 54 nt, Artificial Sequence
  Synthetic V2-5UTR
gggaaauaagagagaaaagaagaguaagaagaaauauaagaccccggcgccacc

SEQ ID NO: 135 - RNA, 9 nt, Artificial Sequence
  Synthetic Kozak consensus sequence
ccrccaugg

SEQ ID NO: 136 - RNA, 47 nt, Artificial Sequence
  Synthetic 5UTR-002
gggagaucagagagaaaagaagaguaagaagaaauauaagagccacc

SEQ ID NO: 137 - RNA, 42 nt, Artificial Sequence
  Synthetic 5UTR-003
gggagacaagcuuggcauuccgguacuguugguaaagccacc

SEQ ID NO: 138 - RNA, 47 nt, Artificial Sequence
  Synthetic 5UTR-008
gggaauuaacagagaaaagaagaguaagaagaaauauaagagccacc

SEQ ID NO: 139 - RNA, 47 nt, Artificial Sequence
  Synthetic 5UTR-009
gggaaauuagacagaaaagaagaguaagaagaaauauaagagccacc

SEQ ID NO: 140 - RNA, 47 nt, Artificial Sequence
  Synthetic 5UTR-010
gggaaauaagagaguaaagaacaguaagaagaaauauaagagccacc

SEQ ID NO: 141 - RNA, 47 nt, Artificial Sequence
  Synthetic 5UTR-011
gggaaaaaagagagaaaagaagacuaagaagaaauauaagagccacc

SEQ ID NO: 142 - RNA, 47 nt, Artificial Sequence
  Synthetic 5UTR-012
gggaaauaagagagaaaagaagaguaagaagauauauaagagccacc

SEQ ID NO: 143 - RNA, 47 nt, Artificial Sequence
  Synthetic 5UTR-013
gggaaauaagagacaaaacaagaguaagaagaaauauaagagccacc

SEQ ID NO: 144 - RNA, 47 nt, Artificial Sequence
  Synthetic 5UTR-014
gggaaauuagagaguaaagaacaguaaguagaauuaaaagagccacc

SEQ ID NO: 145 - RNA, 47 nt, Artificial Sequence
  Synthetic 5UTR-015
gggaaauaagagagaauagaagaguaagaagaaauauaagagccacc

SEQ ID NO: 146 - RNA, 47 nt, Artificial Sequence
  Synthetic 5UTR-016
gggaaauaagagagaaaagaagaguaagaagaaaauuaagagccacc

SEQ ID NO: 147 - RNA, 47 nt, Artificial Sequence
  Synthetic 5UTR-017
gggaaauaagagagaaaagaagaguaagaagaaauuuaagagccacc

SEQ ID NO: 148 - protein, 420 aa, Artificial Sequence
  Synthetic linker
  misc_feature: For every “GGGGGGGGGG GGGGGGGGGG S”, at least one G is present and may or may not repeat up to 20 times
  misc_feature: For every “GnS”, at least one is present and it may or may not repeat up to 20 times
GGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGS

SEQ ID NO: 149 - protein, 40 aa, Artificial Sequence
  Synthetic linker
  misc_feature: At least one G is present and it may or may not repeat up to 40 times
GGGGGGGGGGGGGGGGGGGGGGGGGGGGGGGGGGGGGGGG

SEQ ID NO: 150 - DNA, 30 bp, Artificial Sequence
  Synthetic GC-motif
  misc_feature: [CCG] is present at least once and may or may not repeat up to 10 times
ccgccgccgccgccgccgccgccgccgccg

(19) United States
(12) Patent Application Publication
(10) Pub. No.: US 2023/0081530 A1
(43) Pub. Date: Mar. 16, 2023

(54) METHODS AND COMPOSITIONS FOR TREATING CANCER USING MRNA THERAPEUTICS

(71) Applicant: ModernaTX, Inc., Cambridge, MA (US)

(72) Inventors: Joshua FREDERICK, Charlestown, MA (US); Sushma GURUMURTHY, Sharon, MA (US); Ankita MISHRA, Cambridge, MA (US); Ailin BAI, Newton, MA (US)

(21) Appl. No.: 17/275,832

(22) PCT Filed: Sep. 13, 2019
(86) PCT No.: PCT/US2019/051072

§ 371 Date: Mar. 12, 2021

Related U.S. Application Data
(60) Provisional application No. 62/731,335, filed on Sep. 14, 2018.
(60) Provisional application No. 62/770,024, filed on Nov. 20, 2018.
(60) Provisional application No. 62/881,322, filed on Jul. 31, 2019.

(51) Int. Cl. A61K 31/7105; A61K 9/51; A61K 38/17; A61K 39/395; A61K 38/20; A61P 35/00; A61P 35/02

(52) U.S. Cl. CPC A61K 31/7105; A61K 9/5123; A61K 38/177; A61P 35/02; A61K 38/208; A61K 38/2086; A61P 35/00; A61K 39/3955

(57) ABSTRACT

The disclosure features methods for treating cancer, including solid tumors and disseminated cancers such as myeloid malignancies, using one or more mRNAs encoding an OX40L polypeptide, an IL-12 polypeptide, an IL-15 polypeptide, and combinations thereof.

### RELATED APPLICATIONS

This application claims the benefit of U.S. Provisional Application Ser. No. 62/731,335, filed on Sep. 14, 2018, U.S. Provisional Application Ser. No. 62/770,024, filed on Nov. 20, 2018, and U.S. Provisional Application No. 62/881,322, filed on Jul. 31, 2019. The entire contents of the above-referenced applications are incorporated herein by this reference.

### BACKGROUND OF THE DISCLOSURE

Cancer is a disease characterized by uncontrolled cell division and growth within the body. In the United States, roughly a third of all women and half of all men will experience cancer in their lifetime. Cancers can generally be divided into two categories, solid tumors and disseminated cancers. Each type requires different considerations for developing effective therapeutic approaches.

Disseminated cancers, such as myeloid malignancies, represent a significant population of cancers, with over 30,000 new cases diagnosed each year in the U.S. alone and only about a 20% five-year survival rate. There are approximately 60,000-170,000 cases of myelodysplastic syndrome (MDS), which can progress to acute myeloid leukemia (AML). Treatment options for disseminated cancers such as myeloid malignancies, including AML, are limited, with conventional approaches such as chemotherapy and/or immunomodulatory cytokines or antibodies not being very effective in AML. For example, interleukin-2 treatment alone was found not to be effective for remission maintenance therapy in AML patients (Buyse, M. et al. (2000) Blood 117:26). Similarly, the drug linomide was found to have no benefit over placebo in AML patients after autologous bone marrow transfer (Simonsson, B. et al. (2011) Bone Marrow Transpl. 25:1121). Despite a variety of treatment options, prognosis in patients with relapsed or refractory AML is generally poor.

The treatment of solid tumors includes surgery, chemotherapy and/or radiotherapy. In surgery, most of the tumor or even the invaded organ is excised. Chemotherapy includes the use of drugs to destroy cancer cells. Some cancers are curable by chemotherapy while others are not. Chemotherapeutic drugs can affect not only cancer cells but also other rapidly dividing normal cells such as those in the gastrointestinal tract, bone marrow, hair follicles, and reproductive system which result in several side effects. Radiotherapy includes the use of x-rays to treat cancers. Some are curable by radiotherapy while others are not. With the host of undesired consequences brought about by standard treatments such as chemotherapy and radiotherapy used today, genetic therapy and immunotherapy approaches provide a more targeted approach to disease diagnosis, treatment and management. Therefore, there is a need for improved therapeutic approaches to treat cancer, including solid tumors and disseminated cancers, e.g., myeloid malignancies such as AML.

### SUMMARY OF THE DISCLOSURE

The present disclosure relates to methods and compositions for treating cancer in a subject. The disclosure is based, at least in part, upon the discovery that administration of a combination of mRNAs encoding one or more cell-associated cytokines (e.g., IL-12 and IL-15) and cell-associated costimulatory molecules (e.g., OX40L) induce T cell activation, NK cell activation or both T cell and NK cell activation, resulting in anti-tumor efficacy in solid tumors and disseminated cancers, such as myeloid malignancies (e.g., AML). It was also discovered that anti-tumor efficacy is enhanced by administration of a combination of mRNAs encoding two cell-associated cytokines (e.g., IL-12 and IL-15) with an mRNA encoding a costimulatory molecule (e.g., OX40L). The combination of mRNAs provides various signals to effectively induce T cell activation, NK cell activation or both T cell and NK cell activation.

Moreover, the disclosure provides mRNAs encoding trans-presenting IL-15. IL-15 is a unique cytokine that primarily exists bound to its high affinity receptor, IL-15Rα. IL-15/IL-15Rα complexes are shuttled to the cell surface to stimulate opposing cells through the β/γC receptor complex. Accordingly, without wishing to be bound by theory, mRNA encoding IL-15 and IL-15Rα (together, e.g., as a single mRNA encoding a polypeptide fusion of IL-15 operably linked to IL-15Rα, or separately, e.g., as two separate mRNAs each encoding IL-15 and IL-15Rα) results in trans-presentation, thereby stimulating opposing cells having the β/γC receptor complex.

Without wishing to be bound by theory, a combination of mRNAs encoding one or more cell-associated cytokines and a costimulatory molecule provide enhanced anti-tumor efficacy for solid tumors and disseminated cancers, including myeloid malignancies, relative to a soluble, or secreted form of the same cytokine(s) due to their ability to form a stronger cancer cell:immune cell synapse, and to provide enhanced, prolonged or continuous activation of the cells with which they interact (e.g., T cells and NK cells). T cell activation requires three signals: signal 1 provided by interaction of MHC with a peptide; signal 2 provided by costimulatory molecules, such as OX40L; and signal 3 provided by immune potentiating molecules, including cytokines, such as IL-12 and IL-15. To establish anti-tumor immunity driven by T cells, all three signals are required. However, the tumor microenvironment often fails to provide the necessary signals to activate T cells and potentiate an anti-tumor immune response. By providing an mRNA encoding a costimulatory molecule, such as human OX40L, in combination with an mRNA encoding one or more cell-associated immune potentiating molecules, e.g., mRNA encoding one or more cell-associated cytokines providing signal 3 (e.g., human IL-12, human IL-15/IL-15Rα), to a T cell in a tumor microenvironment by expression of the mRNAs by a cell (e.g., a leukemic cell or antigen presenting cell, such as a dendritic cell), T cells are activated to induce an anti-tumor immune response. Further, by restricting exposure of the cytokines and costimulatory molecules to the cells that express the mRNA, systemic exposure, and potentially undesirable toxicity, is avoided.

With regards to AML, it is known that leukemic cells have reduced and/or downregulated expression of the costimulatory molecules CD80 and CD86 (Yaho, S. and Chen, L., Eur J. Immunol. 2013, 43(3): 576-579; and Hirano N, et al., Leukemia. 1996, 10:1168-1176). By administering an mRNA encoding human OX40L, a strong costimulatory signal is provided where it may be absent or downregulated, or if not absent, such as on dendritic cells, may augment or enhance existing costimulatory signals to provide a stronger synapse between T cells and leukemic and/or dendritic cells expressing the mRNA encoding OX40L and induce an anti-tumor immune response by T cells. As described herein, despite reducing the amount of mRNA encoding the costimulatory molecule (e.g., mRNA encoding OX40L) by 1/10^{th}, a strong anti-tumor effect resulted, compared to treatment without the co-stimulatory molecule, thus demonstrating the theory as described herein.

Significantly, systemic administration of a combination of mRNAs encoding one or more cell-associated cytokines (e.g., human IL-12, human IL-15/IL-15Rα) and an mRNA encoding a costimulatory molecule (e.g., human OX40L) induced a durable anti-cancer memory response in a disseminated cancer model that engrafts in hematopoietic tissues in a manner similar to that seen in AML patients. The anti-cancer immune response prevented relapse and recurrence of disease in this model. This effect is believed to be the first demonstration of an anti-cancer memory response by administration of mRNA therapeutics in a disseminated cancer model.

Also demonstrated herein is an unexpected efficacy of a fractionated dosing regimen. It was discovered that the same dose of mRNAs fractionated into multiple doses, i.e., more than two, and administered during the same treatment period as a single, weekly dose, provided enhanced anti-cancer efficacy in the disseminated cancer model, relative to a single dose, including the induction of a durable anti-cancer memory response. Without wishing to be bound by theory, such fractionated dosing may provide greater or enhanced exposure to the mRNA encoded polypeptides in a subject, resulting in enhanced anti-tumor efficacy with reduced toxicity and better tolerability.

As described herein, biodistribution studies indicate that not only do the mRNA encapsulated lipid nanoparticles transfect leukemic cells, but a variety of immune cells, including dendritic cells. These studies suggest that the combination therapy of one or more mRNAs encoding cell-associated cytokines and an mRNA encoding a costimulatory molecule, such as human OX40L, would be useful for treating a variety of disseminated cancers, including cancers having significant myeloid populations such as AML, as well as multiple myeloma and B cell leukemias.

The combination therapy described herein was also demonstrated to be efficacious in establishing anti-tumor immunity in solid tumors in animal models with various tumor microenvironments. Without being bound by theory, it is believed that the anti-tumor efficacy observed in immune checkpoint resistant tumor models and immunosuppressive tumor models demonstrates the effectiveness of T, NK, NKT and dendritic cell activation following administration of one or more mRNAs encoding cell-associated cytokines and an mRNA encoding a costimulatory molecule, such as human OX40L. As disclosed herein, administration of mRNAs encoding one or more cell-associated cytokines (e.g., human IL-12, human IL-15/IL-15Rα) and an mRNA encoding a costimulatory molecule (e.g., human OX40L) activated and increased innate and adaptive immune cell populations in mice and non-human primates (NHP), as well as induced expression of immunostimulatory cytokines (e.g., IFN-γ and CXCL) in NHPs. As disclosed herein, although DC cell populations were activated and expanded upon administration with the mRNA combination, functional DCs are not required for anti-tumor efficacy in an animal model of AML having defective DC function. The anti-tumor efficacy mediated by the administration of one or more mRNAs encoding cell-associated cytokines and an mRNA encoding a costimulatory molecule, such as human OX40L, requires CD8+ T cells, CD4+ T cells, as well as IFN-γ, as experiments in animals depleted of CD8+ T cells, CD4+ T cells, or IFN-γ resulted in increased disease burden and decreased survival.

Moreover, intratumoral administration of the combination therapy in solid tumors was found to induce an abscopal effect, indicating an anti-tumor immune response which is efficacious against distal, untreated tumors.

Accordingly, provided herein are compositions and methods for treating cancer (e.g., solid tumors and/or disseminated cancers) by providing a combination of two or more mRNA(s) encoding cell-associated cytokines and an mRNA encoding a costimulatory molecule which activate particular immune cells, thereby enhancing an immune response against the cancer. Cancers, including myeloid malignancies, such as AML, are known to evade immune responses by a variety of mechanisms. The compositions and methods of the disclosure are useful for activating innate immunity, activating adaptive immunity and/or activating memory responses against a cancer, e.g., a solid tumor and/or a disseminated cancer, e.g., a myeloid malignancy, such as AML. In some aspects, the mRNA is a modified mRNA.

Accordingly, in some aspects the disclosure provides a method of treating cancer in a human patient, comprising administering to the patient:

(i) a first mRNA encoding human OX40L; and

(ii) at least one second mRNA encoding an immune potentiator, wherein the immune potentiator is a cell-associated cytokine that activates T cells, NK cells, or both T cells and NK cells.

In some aspects the disclosure provides a method of treating cancer in a human patient, comprising administering to the patient:

(i) a first mRNA encoding human OX40L; and

(ii) at least one second mRNA encoding an immune potentiator, wherein the immune potentiator is a cell-associated cytokine that activates T cells, NK cells, or both T cells and NK cells,
wherein the first mRNA and at least one second mRNA are encapsulated in the same or different lipid nanoparticles.

In any of the foregoing or related aspects, the at least one second mRNA is:

(i) an mRNA encoding a trans-presented human IL-15;

(ii) an mRNA encoding a human IL-12 polypeptide operably linked to a membrane domain comprising a transmembrane domain; or

(iii) an mRNA encoding a trans-presented human IL-15 and an mRNA encoding a human IL-12 polypeptide operably linked to a membrane domain.

In some aspects the disclosure provides a method of treating cancer in a subject in need thereof, comprising administering at least two mRNAs selected from the group consisting of:

(i) an mRNA encoding a human OX40L polypeptide;

(ii) an mRNA encoding a human IL-15 polypeptide operably linked to a human IL-15Rα polypeptide;

(iii) an mRNA encoding a human IL-15 polypeptide and an mRNA encoding a human IL-15Rα polypeptide; and

(iv) an mRNA encoding a human IL-12 polypeptide operably linked to a membrane domain comprising a transmembrane domain.

In any of the foregoing or related aspects, the cancer is a disseminated cancer and the first mRNA and the at least one second mRNA are administered systemically. In some aspects, the disseminated cancer is a hematological cancer. In some aspects, the disseminated cancer is a myeloid malignancy. In some aspects, the myeloid malignancy is selected from the group consisting of myeloidysplastic syndrome (MDS), myeloproliferative disorder (MPD) and acute myeloid leukemia (AML). In other aspects, the cancer is a solid tumor and wherein the first mRNA and the at least one second mRNA are administered intratumorally.

In other aspects, the disclosure provides a method of treating a solid tumor in a subject in need thereof, comprising administering (e.g., intratumorally) at least two mRNAs selected from the group consisting of:

(i) an mRNA encoding a human OX40L polypeptide;

(ii) an mRNA encoding a human IL-15 polypeptide operably linked to a human IL-15Rα polypeptide;

(iii) an mRNA encoding a human IL-15 polypeptide and an mRNA encoding a human IL-15Rα polypeptide; and

(iv) an mRNA encoding a human IL-12 polypeptide operably linked to a membrane domain comprising a transmembrane domain.

In some aspects, the disclosure provides a method of treating a disseminated cancer in a subject in need thereof, comprising administering (e.g., systemically, e.g., by intravenous injection) at least two mRNAs selected from the group consisting of:

(i) an mRNA encoding a human OX40L polypeptide;

(ii) an mRNA encoding a human IL-15 polypeptide operably linked to a human IL-15Rα polypeptide;

(iii) an mRNA encoding a human IL-15 polypeptide and a human mRNA encoding an IL-15Rα polypeptide; and

(iv) an mRNA encoding a human IL-12 polypeptide operably linked to a membrane domain comprising a transmembrane domain.

In some aspects, the disclosure provides a method of treating a myeloid malignancy in a subject in need thereof, comprising administering (e.g., systemically, e.g., by intravenous injection) at least two mRNAs selected from the group consisting of:

(i) an mRNA encoding a human OX40L polypeptide;

(ii) an mRNA encoding a human IL-15 polypeptide operably linked to a human IL-15Rα polypeptide;

(iii) an mRNA encoding a human IL-15 polypeptide and an mRNA encoding a human IL-15Rα polypeptide; and

(iv) an mRNA encoding a human IL-12 polypeptide operably linked to a membrane domain comprising a transmembrane domain.

In any of the foregoing or related aspects, the at least two mRNAs are selected from the group consisting of:

(i) an mRNA encoding a human OX40L polypeptide and an mRNA encoding a human IL-12 polypeptide operably linked to a membrane domain comprising a transmembrane domain;

(ii) an mRNA encoding a human OX40L polypeptide, an mRNA encoding a human IL-15 polypeptide and an mRNA encoding a human IL-15Rα polypeptide;

(iii) an mRNA encoding a human OX40L polypeptide and an mRNA encoding a human IL-15 polypeptide operably linked to a human IL-15Rα polypeptide;

(iv) an mRNA encoding a human IL-15 polypeptide, an mRNA encoding a human IL-15Rα polypeptide, and an mRNA encoding a human IL-12 polypeptide operably linked to a membrane domain comprising a transmembrane domain;

(v) an mRNA encoding a human IL-15 polypeptide operably linked to a human IL-15Rα polypeptide, and an mRNA encoding a human IL-12 polypeptide operably linked to a membrane domain comprising a transmembrane domain;

(vi) an mRNA encoding a human OX40L polypeptide, an mRNA encoding a human IL-15 polypeptide, an mRNA encoding a human IL-15Rα polypeptide and an mRNA encoding a human IL-12 polypeptide operably linked to a membrane domain comprising a transmembrane domain; and

(vii) an mRNA encoding a human OX40L polypeptide, an mRNA encoding a human IL-15 polypeptide operably linked to a human IL-15Rα polypeptide, and an mRNA encoding a human IL-12 polypeptide operably linked to a membrane domain comprising a transmembrane domain.

In some aspects, the disclosure provides a method for treating a disseminated cancer in a human patient, comprising systemically administering to the patient:

(i) a first mRNA encoding human OX40L; and

(ii) at least one second mRNA encoding an immune potentiator, wherein the immune potentiator is a cell-associated cytokine that activates T cells, NK cells, or both T cells and NK cells,
wherein the first mRNA and second mRNA are encapsulated in the same or different lipid nanoparticles. In some aspects, the method comprises administering a third mRNA encoding a second immune potentiator, wherein the immune potentiator is a cell-associated cytokine that activates T cells, NK cells, or both T cells and NK cells. In some aspects, the second mRNA encodes a human IL-12 polypeptide operably linked to a membrane domain comprising a transmembrane domain and the third mRNA encodes a trans-presented human IL-15.

In other aspects, the disclosure provides a method of treating a disseminated cancer in a human patient, comprising systemically administering to the patient a pharmaceutical composition comprising a lipid nanoparticle (LNP) and a pharmaceutically acceptable carrier, wherein the LNP comprises:

(i) a first mRNA encoding human OX40L; and

(ii) at least one second mRNA encoding an immune potentiator, wherein the immune potentiator is a cell-associated cytokine that activates T cells, NK cells, or both T cells and NK cells. In some aspects, the method comprises administering a third mRNA encoding a second immune potentiator, wherein the immune potentiator is a cell-associated cytokine that activates T cells, NK cells, or both T cells and NK cells. In some aspects, the second mRNA encodes a human IL-12 polypeptide operably linked to a membrane domain comprising a transmembrane domain and the third mRNA encodes a trans-presented human IL-15.

In further aspects, the disclosure provides a method of treating a disseminated cancer in a human patient, comprising administering to the patient a dosing regimen comprising:

(i) a first fractionated dose of a pharmaceutical composition comprising a first mRNA encoding human OX40L, and at least one second mRNA encoding an immune potentiator, wherein the immune potentiator is a cell-associated cytokine that activates T cells, NK cells, or both T cells and NK cells, and

(ii) at least one second fractionated dose of the pharmaceutical composition, wherein the first and second fractionated doses increase exposure to the mRNA encoded polypeptides in the patient relative to a single dose of the same amount of mRNA during the same dosing interval, thereby treating the disseminated cancer in the patient. In some aspects, the first fractionated dose and second fractionated dose enhance anti-tumor efficacy of the treatment relative to a single dose of the same amount of mRNA. In some aspects, the first fractionated dose and second fractionated dose enhance anti-tumor efficacy with reduced toxicity and better tolerability. In some aspects, the method comprises administering a third mRNA encoding a second immune potentiator, wherein the immune potentiator is a cell-associated cytokine that activates T cells, NK cells, or both T cells and NK cells. In some aspects, the second mRNA encodes a human IL-12 polypeptide operably linked to a membrane domain comprising a transmembrane domain and the third mRNA encodes a trans-presented human IL-15.

In any of the foregoing or related aspects, the cell-associated cytokine is a human IL-12 polypeptide operably linked to a membrane domain comprising a transmembrane domain. In other aspects, the cell-associated cytokine is a trans-presented human IL-15. In some aspects, the trans-presented human IL-15 is a human IL-15 polypeptide operably linked to a human IL-15Rα polypeptide. In other aspects, the trans-presented human IL-15 is encoded by a first mRNA encoding a human IL-15 polypeptide and a second mRNA encoding a human IL-15Rα polypeptide.

In any of the foregoing or related aspects, the mRNA is formulated in the same lipid nanoparticle (LNP). In any of the foregoing or related aspects, each mRNA is formulated in the same LNP. In other aspects, each mRNA is formulated in a separate LNP.

In some aspects, the mRNAs of the disclosure are formulated in the same or different LNP(s), wherein the LNP comprises a molar ratio of about 20-60% ionizable amino lipid: 5-25% phospholipid: 25-55% structural lipid; and 0.5-15% PEG-modified lipid. In yet other aspects, the LNP comprises a molar ratio of about 50% ionizable lipid: about 10% phospholipid: about 38.5% sterol; and about 1.5% PEG-modified lipid. In further aspects, the LNP comprises a molar ratio of 50:38.5:10:1.5 of ionizable lipid: cholesterol: DSPC: PEG-modified lipid.

In some aspects, the mRNAs of the disclosure are formulated in the same or different LNP(s), wherein the LNP comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid. In other aspects, the LNP comprises about 50 mol % ionizable lipid, about 10 mol % non-cationic helper lipid or phospholipid, about 38.5 mol % sterol or other structural lipid, and about 1.5 mol % PEG lipid.

In any of the foregoing or related aspects, the ionizable lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319). In some aspects, the ionizable lipid comprises Compound X.

In any of the foregoing or related aspects, the LNP comprises a molar ratio of about 20-60% Compound X: 5-25% phospholipid: 25-55% cholesterol; and 0.5-15% PEG-modified lipid. In some aspects, the LNP comprises a molar ratio of about 50% Compound X: about 10% phospholipid: about 38.5% cholesterol; and about 1.5% PEG-modified lipid.

In any of the foregoing or related aspects, the PEG-modified lipid is PEG-DMG or Compound P-428. In some aspects, the LNP comprises a molar ratio of 50:38.5:10:1.5 of Compound X:cholesterol:phospholipid:Compound P-428, or of Compound X: cholesterol: DSPC: Compound P-428. In other aspects, the LNP comprises a molar ratio of 40:38.5:20:1.5 of Compound X:cholesterol:phospholipid:Compound P-428, or of Compound X:cholesterol:DSPC:Compound P-428.

In any of the foregoing or related aspects, the LNP comprises a phytosterol or a combination of a phytosterol and cholesterol. In some aspects, the phytosterol is selected from the group consisting of β-sitosterol, stigmasterol, β-sitostanol, campesterol, brassicasterol, and combinations thereof. In some aspects, the phytosterol comprises (i) a sitosterol or a salt or an ester thereof, or (ii) a stigmasterol or a salt or an ester thereof. In some aspects, the phytosterol is beta-sitosterol

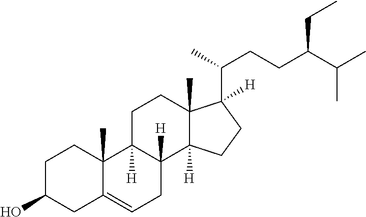

or a salt or an ester thereof.

In other aspects, the phytosterol or a salt or ester thereof is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

In some aspects, the mol % sterol or other structural lipid is 18.5% phytosterol and the total mol % structural lipid is 38.5%. In other aspects, the mol % sterol or other structural lipid is 28.5% phytosterol and the total mol % structural lipid is 38.5%.

In some aspects, the mRNAs of the disclosure are formulated in the same or different LNP(s), wherein the LNP comprises a molar ratio of 50:10:10:28.5:1.5 of Compound X:DSPC:cholesterol:beta-sitosterol:PEG-DMG. In some aspects, the mRNAs of the disclosure are formulated in the same or different LNP(s), wherein the LNP comprises a molar ratio of 50:10:20:18.5:1.5 of Compound X:DSPC:cholesterol:beta-sitosterol:PEG-DMG. In any of the foregoing or related aspects, the cancer is a disseminated cancer. In some aspects, the disseminated cancer is a hematological cancer. In some aspects, the disseminated cancer is a myeloid malignancy.

In any of the foregoing or related aspects, the myeloid malignancy is selected from the group consisting of myelodysplastic syndrome (MDS), myeloproliferative disorder (MPD) and acute myeloid leukemia (AML). In some aspects, the myeloid malignancy is AML.

In any of the foregoing or related aspects, the cancer is a solid tumor. In some aspects, the solid tumor is unresponsive to checkpoint inhibitor therapy. In some aspects, the solid tumor comprises an immunosuppressive tumor microenvironment.

In any of the foregoing or related aspects, the at least two mRNAs are administered intratumorally. In some aspects, the at least two mRNAs are administered intravenously. In some aspects, the mRNAs are encapsulated in the same LNP and formulated in a solution suitable for intratumoral injection. In some aspects, the mRNAs are encapsulated in the same LNP and formulated in a solution suitable for intravenous injection. In other aspects, each mRNA is encapsulated in one or more separate LNPs and formulated in a solution suitable for intratumoral injection. In other aspects, each mRNA is encapsulated in one or more separate LNPs and formulated in a solution suitable for intravenous injection.

In any of the foregoing or related aspects, the method for treating a cancer (e.g., solid tumor or disseminated cancer such as a myeloid malignancy) further comprises administering a checkpoint inhibitor polypeptide. In some aspects, the checkpoint inhibitor polypeptide inhibits PD-1, PD-L¹, CTLA-4, or a combination thereof. In some aspects, the checkpoint inhibitor polypeptide is an antibody or an mRNA encoding the antibody. In some aspects, the antibody is an anti-CTLA-4 antibody or antigen-binding fragment thereof that specifically binds CTLA-4, an anti-PD-1 antibody or antigen-binding fragment thereof that specifically binds PD-1, an anti-PD-L¹ antibody or antigen-binding fragment thereof that specifically binds PD-L¹, and a combination thereof. In some aspects, the anti-PD-L¹ antibody is atezolizumab, avelumab, or durvalumab. In some aspects, the anti-CTLA-4 antibody is tremelimumab or ipilimumab. In some aspects, the anti-PD-1 antibody is nivolumab or pembrolizumab.

In other aspects, the disclosure provides an LNP comprising:

(i) a first mRNA encoding human OX40L; and

(ii) at least one second mRNA encoding an immune potentiator, wherein the immune potentiator is a cell-associated cytokine that activates T cells, NK cells, or both T cells and NK cells.

In other aspects, the disclosure provides an LNP comprising:

(i) an ionizable lipid;

(ii) a sterol or other structural lipid;

(iii) a first mRNA encoding human OX40;

(iv) at least one second mRNA encoding an immune potentiator, wherein the immune potentiator is a cell-associated cytokine that activates T cells, NK cells, or both T cells and NK cells;

(v) optionally, a non-cationic helper lipid or phospholipid; and

(vi) optionally, a PEG-lipid.

In some aspects, the disclosure provides an LNP comprising at least two encapsulated messenger RNAs (mRNAs), wherein the at least two mRNAs are selected from the group consisting of:

(i) an mRNA encoding a human OX40L polypeptide;

(ii) an mRNA encoding a human IL-15 polypeptide operably linked to a human IL-15Rα polypeptide;

(iii) an mRNA encoding a human IL-15 polypeptide and an mRNA encoding a human IL-15Rα polypeptide; and

(iv) an mRNA encoding a human IL-12 polypeptide operably linked to a membrane domain comprising a transmembrane domain.

In some embodiments, the disclosure provides a lipid nanoparticle, wherein the mRNAs are co-formulated in the same lipid nanoparticle, and wherein the mRNAs encoding human OX40L, tethered human IL-12 and cell-associated human IL-15 are co-formulated at a weight (mass) ratio of 1:1:1.

In some embodiments, the mRNAs of the disclosure are co-formulated in the same lipid nanoparticle, wherein the mRNAs encoding human OX40L, tethered human IL-12 and cell-associated human IL-15 are co-formulated at a weight (mass) ratio of 1:1:1, and wherein the mRNA encoding cell-associated human IL-15 is encoded by two mRNAs encoding human IL-15 and human IL-15Rα, and wherein the two mRNAs are co-formulated at a molar ratio of 1:1.

In some aspects, the at least two mRNAs are selected from the group consisting of:

(i) an mRNA encoding a human OX40L polypeptide and an mRNA encoding a human IL-12 polypeptide operably linked to a membrane domain comprising a transmembrane domain;

(ii) an mRNA encoding a human OX40L polypeptide, an mRNA encoding a human IL-15 polypeptide and an mRNA encoding a human IL-15Rα polypeptide;

(iii) an mRNA encoding a human OX40L polypeptide and an mRNA encoding a human IL-15 polypeptide operably linked to a human IL-15Rα polypeptide;

(iv) an mRNA encoding a human IL-15 polypeptide, an mRNA encoding a human IL-15Rα polypeptide, and an mRNA encoding a human IL-12 polypeptide operably linked to a membrane domain comprising a transmembrane domain;

(v) an mRNA encoding a human IL-15 polypeptide operably linked to a human IL-15Rα polypeptide, and an mRNA encoding a human IL-12 polypeptide operably linked to a membrane domain comprising a transmembrane domain;

(vi) an mRNA encoding a human OX40L polypeptide, an mRNA encoding a human IL-15 polypeptide, an mRNA encoding a human IL-15Rα polypeptide and an mRNA encoding a human IL-12 polypeptide operably linked to a membrane domain comprising a transmembrane domain; and

(vii) an mRNA encoding a human OX40L polypeptide, an mRNA encoding a human IL-15 polypeptide operably linked to a human IL-15Rα polypeptide, and an mRNA encoding a human IL-12 polypeptide operably linked to a membrane domain comprising a transmembrane domain.

In some aspects, the disclosure provides a lipid nanoparticle comprising: an mRNA encoding a human OX40L polypeptide, an mRNA encoding a human IL-15 polypeptide, an mRNA encoding a human IL-15Rα polypeptide and an mRNA encoding a human IL-12 polypeptide operably linked to a membrane domain comprising a transmembrane domain.

In some aspects, the disclosure provides a composition comprising: a first lipid nanoparticle encapsulating an mRNA encoding a human OX40L polypeptide, a second lipid nanoparticle encapsulating an mRNA encoding a human IL-15 polypeptide; a third lipid nanoparticle encapsulating an mRNA encoding a human IL-15Rα polypeptide; and a fourth lipid nanoparticle encapsulating an mRNA encoding a human IL-12 polypeptide operably linked to a membrane domain comprising a transmembrane domain.

In some aspects, the disclosure provides a composition comprising: a first lipid nanoparticle encapsulating an mRNA encoding a human OX40L polypeptide, a second lipid nanoparticle encapsulating an mRNA encoding a human IL-15 polypeptide operably linked to an IL-15Rα polypeptide; and a third lipid nanoparticle encapsulating an mRNA encoding a human IL-12 polypeptide operably linked to a membrane domain comprising a transmembrane domain.

In any of the foregoing or related aspects, the mRNA encoding a human OX40L polypeptide comprises the amino acid sequence set forth in SEQ ID NO: 1, or an amino acid sequence having at least 90% identity to the amino acid sequence set forth in SEQ ID NO: 1. In some aspects, the OX40L polypeptide is encoded by a nucleotide sequence comprising the nucleotide sequence set forth SEQ ID NO: 11, or a nucleotide sequence having at least 80% identity to the nucleotide sequence set forth SEQ ID NO: 11.

In any of the foregoing or related aspects, the mRNA encoding a human IL-12 polypeptide operably linked to a membrane domain comprising a transmembrane domain comprises an IL-12 p40 subunit (IL-12B) polypeptide operably linked to an IL-12 p35 subunit (IL-12A) polypeptide. In some aspects, the IL-12B polypeptide is operably linked to the IL-12A polypeptide by a peptide linker. In some aspects, the IL-12 polypeptide comprises the amino acid sequence set forth in SEQ ID NO: 39 or SEQ ID NO: 40, or an amino acid sequence having at least 90% identity to the amino acid sequence set forth in SEQ ID NO: 39 or SEQ ID NO: 40. In some aspects, the IL-12 polypeptide is encoded by a nucleotide sequence comprising the nucleotide sequence set forth SEQ ID NO: 46, or a nucleotide sequence having at least 80% identity to the nucleotide sequence set forth SEQ ID NO: 46. In some aspects the IL-12B polypeptide is located at the 5′ terminus of the IL-12A polypeptide, or the 5′ terminus of the peptide linker; or wherein the IL-12A polypeptide is located at the 5′ terminus of the IL-12B polypeptide, or the 5′ terminus of the peptide linker.

In some aspects, the membrane domain is operably linked to the IL-12A polypeptide by a peptide linker. In other aspects, the membrane domain is operably linked to the IL-12B polypeptide by a peptide linker. In some aspects, the transmembrane domain comprises a transmembrane domain derived from a Type I integral membrane protein. In some aspects, the transmembrane domain is selected from the group consisting of: a Cluster of Differentiation 8 (CD8) transmembrane domain, a Platelet-Derived Growth Factor Receptor (PDGFR) transmembrane domain, and a Cluster of Differentiation 80 (CD80) transmembrane domain.

In some aspects, the PDGFR-beta transmembrane domain comprises the amino acid sequence set forth in SEQ ID NO: 42. In some aspects, the CD8 transmembrane domain comprises the amino acid sequence set forth in SEQ ID NO: 41. In some aspects, the CD80 transmembrane domain comprises the amino acid sequence set forth in SEQ ID NO: 43.

In other aspects, the membrane domain comprises an intracellular domain. In some aspects, the intracellular domain is derived from the same polypeptide as the transmembrane domain. In other aspects, the intracellular domain is derived from a different polypeptide than the transmembrane domain is derived from. In some aspects, the intracellular domain is selected from the group consisting of: a PDGFR intracellular domain, a truncated PDGFR intracellular domain, and a CD80 intracellular domain.

In some aspects, the intracellular domain is a PDGFR intracellular domain comprising a PDGFR-beta intracellular domain comprising the amino acid sequence set forth in SEQ ID NO: 48. In some aspects, the intracellular domain is a truncated PDGFR intracellular domain comprising a PDGFR-beta intracellular domain truncated at E570 or G739. In some aspects, the truncated PDGFR-beta intracellular domain truncated at E570 comprises the amino acid sequence set forth in SEQ ID NO: 49. In some aspects, the truncated PDGFR-beta transmembrane truncated at G739 comprises the amino acid sequence set forth in SEQ ID NO: 50. In other aspects, the intracellular domain is a CD80 intracellular domain comprising the amino acid sequence set forth in SEQ ID NO: 47.

In some aspects, the IL-12 polypeptide operably linked to a membrane domain comprises a membrane domain comprising:

(i) a PDGFR-beta transmembrane domain and a PDGFR-beta intracellular domain;

(ii) a PDGFR-beta transmembrane domain and a truncated PDGFR-beta intracellular domain truncated at E570;

(iii) a PDGFR-beta transmembrane domain and a truncated PDGFR-beta intracellular domain truncated at G739; or

(iv) a CD80 transmembrane domain and a CD80 intracellular domain.

In any of the foregoing or related aspects, the mRNA encoding a human IL-15Rα polypeptide comprises a sushi domain. In some aspects, the IL-15Rα polypeptide comprises a sushi domain, an intracellular domain and a transmembrane domain. In some aspects, the intracellular domain and the transmembrane domain are derived from IL-15Rα. In other aspects, the intracellular domain and the transmembrane domain are derived from a heterologous polypeptide.

In some aspects, the mRNA encoding a human IL-15 polypeptide comprises the amino acid sequence set forth in SEQ ID NO: 17, or an amino acid sequence having at least 90% identity to the amino acid sequence set forth in SEQ ID NO: 17. In some aspects, the IL-15 polypeptide is encoded by a nucleotide sequence comprising the nucleotide sequence set forth SEQ ID NO: 122, or a nucleotide sequence having at least 80% identity to the nucleotide sequence set forth SEQ ID NO: 122.

In some aspects, the IL-15Rα polypeptide comprises the amino acid sequence set forth in SEQ ID NO: 13, or an amino acid sequence having at least 90% identity to the amino acid sequence set forth in SEQ ID NO: 13. In some aspects, the IL-15Rα polypeptide is encoded by a nucleotide sequence comprising the nucleotide sequence set forth SEQ ID NO: 22, or a nucleotide sequence having at least 80% identity to the nucleotide sequence set forth SEQ ID NO: 22.

In some aspects, the IL-15 polypeptide operably linked to an IL-15Rα polypeptide comprises the amino acid sequence set forth in any one of SEQ ID NOs: 23, 27 and 123, or an amino acid sequence having at least 90% identity to the amino acid sequence set forth in any one of SEQ ID NOs: 23, 27 and 123. In some aspects, the IL-15 polypeptide operably linked to an IL-15Rα polypeptide is encoded by a nucleotide sequence comprising the nucleotide sequence set forth in any one of SEQ ID NOs: 24-26, 28-30 and 124-126, or a nucleotide sequence having at least 80% identity to the nucleotide sequence set forth in any one of SEQ ID NOs: 24-26, 28-30 and 124-126.

In other aspects, the disclosure provides an LNP comprising:

(i) an mRNA encoding a human OX40L polypeptide, wherein the human OX40L polypeptide comprises the amino acid sequence set forth in SEQ ID NO: 1;

(ii) an mRNA encoding a human IL-15 polypeptide, wherein the human IL-15 polypeptide comprises the amino acid sequence set forth in SEQ ID NO: 17;

(iii) an mRNA encoding a human IL-15Rα polypeptide, wherein the human IL-15Rα polypeptide comprises the amino acid sequence set forth in SEQ ID NO: 13; and

(iv) an mRNA encoding a human IL-12 polypeptide operably linked to a membrane domain comprising a transmembrane domain, wherein the human IL-12 polypeptide comprises the amino acid sequence set forth in SEQ ID NO: 61, wherein the LNP comprises a molar ratio of about 20-60% ionizable amino lipid: 5-25% phospholipid: 25-55% structural lipid; and 0.5-15% PEG-modified lipid. In some embodiments, the mRNAs encoding human OX40L, tethered human IL-12 and cell-associated human IL-15 are co-formulated in the LNP at a weight (mass) ratio of 1:1:1. In some embodiments, the mRNAs encoding human OX40L, tethered human IL-12 and cell-associated human IL-15 are co-formulated at a weight (mass) ratio of 1:1:1, and wherein the mRNA encoding cell-associated human IL-15 is encoded by two mRNAs encoding human IL-15 and human IL-15Rα, and wherein the two mRNAs are co-formulated at a molar ratio of 1:1.

In some aspects, the disclosure provides an LNP comprising:

(i) an mRNA encoding a human OX40L polypeptide, wherein the mRNA comprises the nucleotide sequence set forth in SEQ ID NO: 11, or a nucleotide sequence having at least 80% identity to the nucleotide sequence set forth in SEQ ID NO: 11;

(ii) an mRNA encoding a human IL-15 polypeptide, wherein the mRNA comprises the nucleotide sequence set forth in SEQ ID NO: 122, or a nucleotide sequence having at least 80% identity to the nucleotide sequence set forth in SEQ ID NO: 122;

(iii) an mRNA encoding a human IL-15Rα polypeptide, wherein the mRNA comprises the nucleotide sequence set forth in SEQ ID NO: 22, or a nucleotide sequence having at least 80% identity to the nucleotide sequence set forth in SEQ ID NO: 22; and

(iv) an mRNA encoding a human IL-12 polypeptide operably linked to a membrane domain comprising a transmembrane domain, wherein the mRNA comprises the nucleotide sequence set forth in SEQ ID NO: 60, or a nucleotide sequence having at least 80% identity to the nucleotide sequence set forth in SEQ ID NO: 60,
wherein the LNP comprises a molar ratio of about 20-60% ionizable amino lipid: 5-25% phospholipid: 25-55% structural lipid; and 0.5-15% PEG-modified lipid. In some embodiments, the mRNAs encoding human OX40L, tethered human IL-12 and cell-associated human IL-15 are co-formulated in the LNP at a weight (mass) ratio of 1:1:1. In some embodiments, the mRNAs encoding human OX40L, tethered human IL-12 and cell-associated human IL-15 are co-formulated at a weight (mass) ratio of 1:1:1, and wherein the mRNA encoding cell-associated human IL-15 is encoded by two mRNAs encoding human IL-15 and human IL-15Rα, and wherein the two mRNAs are co-formulated at a molar ratio of 1:1.

In one embodiment, the LNP comprises a range of 0.1-1:0.1-1:0.1-1 weight (mass) ratio of OX40L: IL-15+IL-15Rα:IL-12. In one embodiment, the LNP comprises a 1:1:1 weight (mass) ratio of mRNAs encoding OX40L:IL-15+IL-15Rα:IL-12. In some aspects, the LNP comprises a 1:1:1 weight (mass) ratio of mRNAs encoding OX40L:IL-15/IL-15Rα:IL-12. In some aspects, the amount of mRNA encoding human OX40L polypeptide is 1/10^{th} the amount of the remaining mRNAs in the LNP.

In any of the foregoing or related aspects, the LNP is formulated for intratumoral delivery. In other aspects, the LNP is formulated for intravenous delivery.

In some aspects, the LNP comprises a molar ratio of about 20-60% ionizable amino lipid: 5-25% phospholipid: 25-55% structural lipid; and 0.5-15% PEG-modified lipid. In some aspects, the lipid nanoparticle comprises a molar ratio of about 50% ionizable lipid: about 10% phospholipid: about 38.5% sterol; and about 1.5% PEG-modified lipid. In some aspects, the lipid nanoparticle comprises a molar ratio of 50:38.5:10:1.5 of ionizable lipid: cholesterol: DSPC: PEG-modified lipid. In some aspects, the ionizable lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319). In some aspects, the ionizable lipid comprises Compound X.

In some aspects, the LNP comprises a molar ratio of about 20-60% Compound X: 5-25% phospholipid: 25-55% cholesterol; and 0.5-15% PEG-modified lipid. In some aspects, the lipid nanoparticle comprises a molar ratio of about 50% Compound X: about 10% phospholipid: about 38.5% cholesterol; and about 1.5% PEG-modified lipid.

In some aspects, the PEG-modified lipid in the lipid nanoparticle is PEG-DMG or Compound P-428. In some aspects, the lipid nanoparticle comprises a molar ratio of 50:38.5:10:1.5 of Compound X:cholesterol:phospholipid:Compound P-428, or of Compound X:cholesterol:DSPC:Compound P-428. In some aspects, the lipid nanoparticle comprises a molar ratio of 40:38.5:20:1.5 of Compound X: cholesterol: phospholipid: Compound P-428, or of Compound X:cholesterol:DSPC:Compound P-428.

In some aspects, the LNP comprises a phytosterol or a combination of a phytosterol and cholesterol. In some aspects, the phytosterol is selected from the group consisting of β-sitosterol, stigmasterol, β-sitostanol, campesterol, brassicasterol, and combinations thereof. In some aspects, the phytosterol comprises (i) a sitosterol or a salt or an ester thereof, or (ii) a stigmasterol or a salt or an ester thereof. In some aspects, the phytosterol is beta-sitosterol

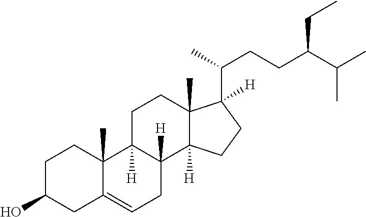

or a salt or an ester thereof.

In other aspects, the phytosterol or a salt or ester thereof is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

In some aspects, the mol % sterol or other structural lipid is 18.5% phytosterol and the total mol % structural lipid is 38.5%. In other aspects, the mol % sterol or other structural lipid is 28.5% phytosterol and the total mol % structural lipid is 38.5%.

In some aspects, the LNP comprises a molar ratio of 50:10:10:28.5:1.5 of Compound X:DSPC:cholesterol:beta-sitosterol:PEG-DMG. In some aspects, the mRNAs of the disclosure are formulated in the same or different LNP(s), wherein the LNP comprises a molar ratio of 50:10:20:18.5:1.5 of Compound X:DSPC:cholesterol:beta-sitosterol:PEG-DMG.

In any of the foregoing aspects, each mRNA comprises a 3′ untranslated region (UTR). In some aspects, the 3′UTR comprises at least one microRNA (miR) binding site. In some aspects, the at least one miR binding site is a miR-122 binding site. In some aspects, the miR-122 binding site is a miR-122-3p or a miR-122-5p binding site. In some aspects, the miR-122-5p binding site comprises the nucleotide sequence set forth in SEQ ID NO: 83. In some aspects, the miR-122-3p binding site comprises the nucleotide sequence set forth in SEQ ID NO: 74. In any of the foregoing aspects, each mRNA comprises a 3′UTR comprising the nucleotide sequence set forth in SEQ ID NO: 77 or SEQ ID NO: 121

In any of the foregoing aspects, each mRNA comprises a 5′ untranslated region (UTR). In some aspects, the 5′UTR comprises the nucleotide sequence set forth in SEQ ID NO: 12 or SEQ ID NO: 133.

In any of the foregoing aspects, each mRNA includes at least one chemical modification. In some aspects, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In any of the foregoing aspects, at least 95% of uridines in each mRNA are N1-methylpseudouridine. In some aspects, at least 99% of uridines in each mRNA are N1-methylpseudouridine. In some aspects, 100% of uridines in each mRNA are N1-methylpseudouridine.

In some aspects, the disclosure provides methods for treating a cancer in a subject in need thereof, the method comprising administering to the subject a lipid nanoparticle as described herein. In some aspects, the disclosure provides methods for treating a disseminated cancer, such as a myeloid malignancy, in a subject in need thereof, the method comprising administering to the subject a lipid nanoparticle as described herein. In some aspects, the disclosure provides methods for treating a solid tumor in a subject in need thereof, the method comprising administering to the subject a lipid nanoparticle as described herein. In some aspects, the method further comprises administering a checkpoint inhibitor polypeptide or an mRNA encoding a checkpoint inhibitor polypeptide. In some aspects, the checkpoint inhibitor polypeptide inhibits PD-1, PD-L¹, CTLA-4, or a combination thereof. In some aspects, the checkpoint inhibitor polypeptide is an antibody or an mRNA encoding the antibody. In some aspects, the antibody is an anti-CTLA-4 antibody or antigen-binding fragment thereof that specifically binds CTLA-4, an anti-PD-1 antibody or antigen-binding fragment thereof that specifically binds PD-1, an anti-PD-L¹ antibody or antigen-binding fragment thereof that specifically binds PD-L¹, and a combination thereof. In some aspects, the anti-PD-L¹ antibody is atezolizumab, avelumab, or durvalumab. In some aspects, the anti-CTLA-4 antibody is tremelimumab or ipilimumab. In some aspects, the anti-PD-1 antibody is nivolumab or pembrolizumab.

In other aspects, the disclosure provides a lipid nanoparticle as described herein, and an optional pharmaceutically acceptable carrier, for use in treating or delaying progression of a cancer in an individual, wherein treatment comprises administration of the lipid nanoparticle. In other aspects, the disclosure provides a lipid nanoparticle as described herein, and an optional pharmaceutically acceptable carrier, for use in treating or delaying progression of a disseminated cancer, such as a myeloid malignancy, in an individual, wherein treatment comprises administration of the lipid nanoparticle. In yet other aspects, the disclosure provides a lipid nanoparticle as described herein, and an optional pharmaceutically acceptable carrier, for use in treating or delaying progression of a solid tumor in an individual, wherein treatment comprises administration of the lipid nanoparticle.

In some aspects, the disclosure provides a lipid nanoparticle as described herein, and an optional pharmaceutically acceptable carrier, for use in treating or delaying progression of a cancer in an individual, wherein treatment comprises administration of the lipid nanoparticle in combination with a composition comprising an immune checkpoint inhibitory polypeptide, and an optional pharmaceutically acceptable carrier. In some aspects, the disclosure provides a lipid nanoparticle as described herein, and an optional pharmaceutically acceptable carrier, for use in treating or delaying progression of a disseminated cancer, such as a myeloid malignancy, in an individual, wherein treatment comprises administration of the lipid nanoparticle in combination with a composition comprising an immune checkpoint inhibitory polypeptide, and an optional pharmaceutically acceptable carrier. In some aspects, the disclosure provides a lipid nanoparticle as described herein, and an optional pharmaceutically acceptable carrier, for use in treating or delaying progression of a solid tumor in an individual, wherein treatment comprises administration of the lipid nanoparticle in combination with a composition comprising an immune checkpoint inhibitory polypeptide, and an optional pharmaceutically acceptable carrier.

In further aspects, the disclosure provides use of a lipid nanoparticle as described herein, and an optional pharmaceutically acceptable carrier, in the manufacture of a medicament for treating or delaying progression of a cancer in an individual, wherein the medicament comprises the lipid nanoparticle, and an optional pharmaceutically acceptable carrier, and wherein the treatment comprises administration of the medicament. In further aspects, the disclosure provides use of a lipid nanoparticle as described herein, and an optional pharmaceutically acceptable carrier, in the manufacture of a medicament for treating or delaying progression of a disseminated cancer, such as a myeloid malignancy, in an individual, wherein the medicament comprises the lipid nanoparticle, and an optional pharmaceutically acceptable carrier, and wherein the treatment comprises administration of the medicament. In further aspects, the disclosure provides use of a lipid nanoparticle as described herein, and an optional pharmaceutically acceptable carrier, in the manufacture of a medicament for treating or delaying progression of a solid tumor in an individual, wherein the medicament comprises the lipid nanoparticle, and an optional pharmaceutically acceptable carrier, and wherein the treatment comprises administration of the medicament.

In some aspects, the disclosure provides use of a lipid nanoparticle as described herein, and an optional pharmaceutically acceptable carrier, in the manufacture of a medicament for treating or delaying progression of a cancer in an individual, wherein the medicament comprises the lipid nanoparticle, and an optional pharmaceutically acceptable carrier, and wherein the treatment comprises administration of the medicament in combination with a composition comprising an immune checkpoint inhibitor polypeptide or an mRNA encoding the immune checkpoint inhibitor polypeptide, and an optional pharmaceutically acceptable carrier. In some aspects, the disclosure provides use of a lipid nanoparticle as described herein, and an optional pharmaceutically acceptable carrier, in the manufacture of a medicament for treating or delaying progression of a disseminated cancer, such as a myeloid malignancy, in an individual, wherein the medicament comprises the lipid nanoparticle, and an optional pharmaceutically acceptable carrier, and wherein the treatment comprises administration of the medicament in combination with a composition comprising an immune checkpoint inhibitor polypeptide or an mRNA encoding an immune checkpoint inhibitor polypeptide, and an optional pharmaceutically acceptable carrier. In some aspects, the disclosure provides use of a lipid nanoparticle as described herein, and an optional pharmaceutically acceptable carrier, in the manufacture of a medicament for treating or delaying progression of a solid tumor in an individual, wherein the medicament comprises the lipid nanoparticle, and an optional pharmaceutically acceptable carrier, and wherein the treatment comprises administration of the medicament in combination with a composition comprising an immune checkpoint inhibitor polypeptide or an mRNA encoding an immune checkpoint inhibitor polypeptide, and an optional pharmaceutically acceptable carrier.

In other aspects, the disclosure provides a kit comprising a container comprising a lipid nanoparticle as described herein, and an optional pharmaceutically acceptable carrier, and a package insert comprising instructions for administration of the lipid nanoparticle for treating or delaying progression of a cancer in an individual. In some aspects, the package insert further comprises instructions for administration of the lipid nanoparticle in combination with a composition comprising an immune checkpoint inhibitor polypeptide, or an mRNA encoding an immune checkpoint inhibitor polypeptide, and an optional pharmaceutically acceptable carrier, for treating or delaying progression of a cancer in an individual.

In other aspects, the disclosure provides a kit comprising a container comprising a lipid nanoparticle as described herein, and an optional pharmaceutically acceptable carrier, and a package insert comprising instructions for administration of the lipid nanoparticle for treating or delaying progression of a disseminated cancer, such as a myeloid malignancy, in an individual. In some aspects, the package insert further comprises instructions for administration of the lipid nanoparticle in combination with a composition comprising an immune checkpoint inhibitor polypeptide, and an optional pharmaceutically acceptable carrier, for treating or delaying progression of a disseminated cancer, such as a myeloid malignancy, in an individual.

In other aspects, the disclosure provides a kit comprising a container comprising a lipid nanoparticle as described herein, and an optional pharmaceutically acceptable carrier, and a package insert comprising instructions for administration of the lipid nanoparticle for treating or delaying progression of a solid tumor in an individual. In some aspects, the package insert further comprises instructions for administration of the lipid nanoparticle in combination with a composition comprising an immune checkpoint inhibitor polypeptide, and an optional pharmaceutically acceptable carrier, for treating or delaying progression of a solid tumor in an individual.

In some aspects, the disclosure provides a kit comprising a container comprising a lipid nanoparticle as described herein, and an optional pharmaceutically acceptable carrier, and a package insert comprising instructions for administration of the medicament alone, or in combination with a composition comprising an immune checkpoint inhibitor polypeptide, and an optional pharmaceutically acceptable carrier, for treating or delaying progression of a cancer in an individual. In some aspects, the disclosure provides a kit comprising a container comprising a lipid nanoparticle as described herein, and an optional pharmaceutically acceptable carrier, and a package insert comprising instructions for administration of the medicament alone, or in combination with a composition comprising an immune checkpoint inhibitor polypeptide, and an optional pharmaceutically acceptable carrier, for treating or delaying progression of a disseminated cancer, such as a myeloid malignancy, in an individual. In some aspects, the disclosure provides a kit comprising a container comprising a lipid nanoparticle as described herein, and an optional pharmaceutically acceptable carrier, and a package insert comprising instructions for administration of the medicament alone, or in combination with a composition comprising an immune checkpoint inhibitor polypeptide, and an optional pharmaceutically acceptable carrier, for treating or delaying progression of a solid tumor in an individual.

In other aspects, the disclosure provides methods for enhancing an immune response in a subject, comprising administering to the subject a lipid nanoparticle or combination of mRNAs as described herein.

In other aspects, the disclosure provides methods for enhancing immune cell activation in a subject, comprising administering to the subject a lipid nanoparticle or combination of mRNAs as described herein. In some aspects, the immune cell activation comprises T cell activation, NK cell activation, or both T cell and NK cell activation.

In other aspects, the disclosure provides methods for enhancing NK cell activation in a subject, comprising administering to the subject a lipid nanoparticle or combination of mRNAs as described herein.

In any of the foregoing methods, the subject has a myeloid malignancy. In some aspects, the myeloid malignancy is AML. In any of the foregoing methods, the subject has a solid tumor.

In any of the foregoing aspects, the method further comprises administering a checkpoint inhibitor polypeptide or an mRNA encoding a checkpoint inhibitor polypeptide as described herein.

In any of the foregoing aspects, the mRNA, pharmaceutical composition or LNP as described herein has one or more activities selected from the group consisting of (a) increasing NK, NKT, CD8+ T, CD4+ T, and/or dendritic cell (DC) populations; (b) increasing proliferation of NK, NKT, CD8+ T cells, CD4+ T cells, and/or DCs; (c) increasing activation of NK, NKT, CD8+ T, CD4+ T, and/or dendritic cells; (d) increasing maturation of DCs; (e) decreasing disease burden in treated subject; (f) increasing survival in treated subject; (g) increasing expression of IFNγ or IP10; and (h) any combinations of (a)-(g).

In any of the foregoing aspects, the DC populations affected by the mRNA, pharmaceutical composition or LNP as described herein are CD8+ cDC1, CD103+ cDC1, cDC2, or iDC populations.

### BRIEF DESCRIPTION OF THE DRAWINGS

FIG. 1 provides graphs showing transfection efficacy of an AML cell line (Kasumi-1) in vitro with lipid nanoparticles (LNPs) containing different PEG-modified lipids (PEG DMG or Compound 428) in the absence or presence of human serum.

FIG. 2 provides graphs showing transfection efficacy of primary AML samples in vitro with LNPs containing different PEG-modified lipids (PEG DMG or Compound 428) in presence of human serum.

FIGS. 3A-3C are graphs showing tumor volume in mice implanted with P388D1 AML cells and treated with an mRNA encoding murine OX40L (mOX40L) formulated in an LNP (FIG. 3A), mRNAs encoding mOX40L and human IL-15 (hIL-15) formulated in an LNP (FIG. 3B) and mRNAs encoding mOX40L, hIL-15 and murine IL-12 (mIL-12) formulated in an LNP (FIG. 3C). LNPs were administered intratumorally.

FIGS. 4A-4D provide schematics of human IL-15/IL-15Rα constructs. FIG. 4A shows an IL-15 polypeptide (left) and an IL-15Rα polypeptide comprising a sushi domain and a transmembrane domain (right), wherein IL-15 binds to IL-15Rα sushi domain with high affinity, thereby restricting IL-15 to IL-15Rα expressing cells. FIG. 4B shows a tethered IL-15 construct, wherein an IL-15 polypeptide is linked to a full-length IL-15Rα, thereby tethering IL-15 to the cell membrane. FIG. 4C shows a secreted IL-15 construct, wherein an IL-15 polypeptide is linked to the sushi domain of IL-15Rα. FIG. 4D shows a tethered IL-15 constructs, wherein an IL-15 polypeptide is linked to the sushi domain of IL-15Rα which is linked to a transmembrane domain and intracellular domain of a heterologous polypeptide (e.g. CD80).

FIGS. 5A-5D provide graphs comparing protein expression and T cell proliferation between human IL-15/IL-15Rα constructs described in FIGS. 4A-4C. FIG. 5A shows protein expression of IL-15 in the supernatant or the lysate when HeLa cells were transfected with mRNA encoding the indicated IL-15/IL-15Rα construct in Lipofectamine 2000. FIG. 5B shows proliferation of T cells when co-cultured with HeLa cells transfected with mRNA encoding the indicated IL-15/IL-15Rα constructs in Lipofectamine 2000. FIG. 5C shows protein expression of IL-15 in the supernatant or the lysate when HeLa cells were transfected with different mRNA versions encoding the indicated IL-15/IL-15Rα constructs. FIG. 5D shows the percent of protein shed in the supernatant (supernatant expression/lysate expression+supernatant expression).

FIGS. 6A-6F are graphs showing tumor volume in mice implanted with C1498 AML cells and treated intratumorally with LNPs encapsulating mRNAs encoding NST-mOX40L (NST) (FIG. 6A), mOX40L (FIG. 6B), hIL-15/IL-15Rα (FIG. 6C), membrane tethered mIL-12 (mIL-12TM) (FIG. 6D), mOX40L+hIL-15/IL-15Rα (FIG. 6E) or mOX40L+hIL-15/IL-15Rα+mIL-12TM (FIG. 6F).

FIGS. 7A-7C are graphs showing percent survival of mice with AML tumors treated intratumorally with LNPs encapsulating mRNAs encoding various single agents (mOX40L or hIL-15/IL-15Rα or mIL-12TM mRNAs) (FIG. 7A), various mOX40L+hIL-15/IL-15Rα doublet mRNAs (FIG. 7B) or various mOX40L+hIL-15/IL-15Rα+mIL-12TM triplet mRNAs (FIG. 7C).

FIGS. 8A-8B show disease burden in mice bearing a disseminated model of AML, and treated intravenously with a combination of mRNAs encoding mouse OX40L (i.e., mOX40L), cell-associated human IL-15 (i.e., hIL-15+hIL-15Rα) and tethered mouse IL-12 (mIL-12 linked to a PDGFR transmembrane domain, i.e., mIL-12TM) (2 mg/kg total mRNA), formulated in separate LNPs comprising Compound X and Compound 428. FIG. 8A shows bioluminescence imaging (BLI), and FIG. 8B shows the number of GFP+ cells in the blood as determined by flow cytometry.

FIG. 9 provides graphs showing a decrease in leukemia burden in blood of mice treated intravenously with a combination of mRNAs encoding mOX40L, cell-associated hIL-15, and tethered mIL-12, formulated in separate LNPs comprising Compound X and Compound 428, 21 days after implant of AML cells. The number of GFP+ cells in the blood was determined (left), along with the % of GFP+ of CD45+ cells (right) by flow cytometry.

FIG. 10 provides a Kaplan-Meier survival graph showing mice from FIG. 9, and mice treated with a combination of mRNAs encoding mOX40L, cell-associated hIL-15 and tethered mIL-12, formulated in separate LNPs comprising Compound X and Compound 428, at varying dosing regimens (i.e., 2 mg/kg once (QD×1); 2 mg/kg once a week for three weeks (Q7D×3); 0.67 mg/kg once a week for three weeks (Q7D×3); 0.22 mg/kg three times a week for three weeks (TIW×3)).

FIG. 11 provides a graph showing protective immunity in mice from FIG. 9 that completely responded to combination therapy of mRNAs encoding mOX40L, cell-associated hIL-15 and tethered mIL-12 at various dosing regimens, and were re-challenged with AML cells, as determined by bioluminescence imaging (BLI).

FIG. 12 provides a Kaplan-Meier survival graph of mice re-challenged with AML cells, as described in FIG. 11.

FIG. 13 provides graphs showing the number of GFP+ cells as determined by flow cytometry in the blood of mice re-challenged with AML cells, as described in FIG. 11.

FIG. 14 provides graphs showing the percentage of mOX40L+ cells in the indicated cell types isolated from the peripheral blood, spleen or bone marrow of mice bearing AML cells 24 hours after intravenous administration of the third TIW dose of 0.22 mg/kg, of an mRNA encoding mOX40L formulated in an LNP comprising Compound X and Compound 428 (LNP1) or an LNP comprising Compound X/DSPC/cholesterol/beta-sitosterol/PEG-DMG (LNP2).

FIG. 15 provides graphs showing serum cytokine levels of mouse IFNγ (left), endogenous mouse IL-15/15R (middle) and mouse IP-10 (right) at 6, 24, 48 and 54 hours after the first intravenous dose from mice that received a combination of mRNAs encoding mOX40L, cell-associated hIL-15 and tethered mIL-12, formulated in separate LNPs, at a dose of either 0.22 mg/kg three times a week (TIW) or 2 mg/kg single dose.

FIG. 16 provides graphs showing serum cytokine levels of mouse IFNγ (left) and endogenous mouse IL-15/IL-15R (right) 6 and 24 hours after the first and second intravenous TIW dose from mice that received mRNA encoding either mOX40L; cell-associated hIL-15; tethered mIL-12; mOX40L+cell-associated hIL-15; mOX40L+tethered mIL-12; cell-associated hIL-15+tethered mIL-12; or mOX40L+cell-associated hIL-15+tethered mIL-12, formulated in LNP1 or LNP2.

FIGS. 17A-17D provide graphs showing the percentage of body weight change in mice bearing a disseminated model of AML, and treated intravenously with mRNAs encoding: mOX40L+cell-associated hIL-15 (FIG. 17A); mOX40L+tethered mIL-12 (FIG. 17B); cell-associated hIL-15+tethered mIL-12 (FIG. 17C); or mOX40L+cell-associated hIL-15+tethered mIL-12 (FIG. 17D). mRNAs were formulated in separate LNPs comprising Compound X and Compound 428, and administered at a dose of 0.22 mg/kg three times a week for three weeks.

FIGS. 18A-18B provide graphs showing tumor volume of mice bearing MC38-R tumors administered a single intratumoral dose of mRNAs encoding mOX40L, tethered mIL-12, and cell-associated hIL-15 (FIG. 18A) in combination with an immune checkpoint inhibitor, i.e., an anti-mCTLA-4 antibody (FIG. 18B).

FIGS. 19A-19D provide flow cytometry plots showing NK cells as a percentage of live CD45+ cells at 24 hours post-1^{st} dose (24h), 24 hours post-3^{rd} dose (6d) and 24 hours post-6^{th} dose (13d) in mice administered the mRNAs encoding mOX40L, tethered mIL-12, and cell-associated hIL-15 or control mRNA (NST). The NK cell percentage in peripheral blood (PB) (FIG. 19A), spleen (SP) (FIG. 19B), bone marrow (BM) (FIG. 19C), and inguinal lymph nodes (LN) (FIG. 19D) are provided.

FIGS. 20A-20D provide flow cytometry plots showing percentage of NK cells expressing the activation marker CD69 at 24 hours post-1^{st} dose (24h), 24 hours post-3^{rd} dose (6d) and 24 hours post-6″ dose (13d) in mice administered mRNAs encoding mOX40L, tethered mIL-12, and cell-associated hIL-15 or control mRNA (NST). The percentage of NK cells expressing CD69 in (PB) (FIG. 20A), spleen (SP) (FIG. 20B), bone marrow (BM) (FIG. 20C), and inguinal lymph nodes (LN) (FIG. 20D) are provided.

FIGS. 21A-21D provide flow cytometry plots showing NKT cells as a percentage of live CD45+ cells at 24 hours post-1^{st} dose (24h), 24 hours post-3^{rd} dose (6d) and 24 hours post-6^{th} dose (13d) in mice administered mRNAs encoding mOX40L, tethered mIL-12, and cell-associated hIL-15 or control mRNA (NST). The NK cell percentage in peripheral blood (PB) (FIG. 21A), spleen (SP) (FIG. 21B), bone marrow (BM) (FIG. 21C), and inguinal lymph nodes (LN) (FIG. 21D) are provided.

FIGS. 22A-22D provide flow cytometry plots showing percentage of NKT cells expressing the activation marker CD69 at 24 hours post-1^{st} dose (24 h), 24 hours post-3^{rd} dose (6 d) and 24 hours post-6th dose (13d) in mice administered the mRNAs encoding mOX40L, tethered mIL-12, and cell-associated hIL-15 or control mRNA (NST). The percentage of NKT cells expressing CD69 in (PB) (FIG. 22A), spleen (SP) (FIG. 22B), bone marrow (BM) (FIG. 22C), and inguinal lymph nodes (LN) (FIG. 22D) are provided.

FIGS. 23A-23D provide flow cytometry plots showing CD8+ T cells as a percentage of live CD45+ cells at 24 hours post-1^{st} dose (24h), 24 hours post-3^{rd} dose (6d) and 24 hours post-6^{th} dose (13d) in mice administered mRNAs encoding mOX40L, tethered mIL-12, and cell-associated hIL-15 or control mRNA (NST). The CD8+ T cell percentage in peripheral blood (PB) (FIG. 23A), spleen (SP) (FIG. 23B), bone marrow (BM) (FIG. 23C), and inguinal lymph nodes (LN) (FIG. 23D) are provided.

FIGS. 24A-24D provide flow cytometry plots showing percentage of CD8+ T cells expressing the activation marker CD69 at 24 hours post-1^{st} dose (24h), 24 hours post-3^{rd} dose (6d) and 24 hours post-6^{th} dose (13d) in mice administered mRNAs encoding mOX40L, tethered mIL-12, and cell-associated hIL-15 or control mRNA (NST). The percentage of CD8+ T cells expressing CD69 in (PB) (FIG. 24A), spleen (SP) (FIG. 24B), bone marrow (BM) (FIG. 24C), and inguinal lymph nodes (LN) (FIG. 24D) are provided.

FIGS. 25A-25D provide flow cytometry plots showing CD4+ T cells as a percentage of live CD45+ cells at 24 hours post-1^{st} dose (24h), 24 hours post-3^{rd} dose (6d) and 24 hours post-6^{th} dose (13d) in mice administered mRNAs encoding mOX40L, tethered mIL-12, and cell-associated hIL-15 or control mRNA (NST). The CD4+ T cell percentage in peripheral blood (PB) (FIG. 25A), spleen (SP) (FIG. 25B), bone marrow (BM) (FIG. 25C), and inguinal lymph nodes (LN) (FIG. 25D) are provided.

FIGS. 26A-26D provide flow cytometry plots showing percentage of CD4+ T cells expressing the activation marker CD69 at 24 hours post-1^{st} dose (24h), 24 hours post-3^{rd} dose (6d) and 24 hours post-6^{th} dose (13d) in mice administered mRNAs encoding mOX40L, tethered mIL-12, and cell-associated hIL-15 or control mRNA (NST). The percentage of CD4+ T cells expressing CD69 in (PB) (FIG. 26A), spleen (SP) (FIG. 26B), bone marrow (BM) (FIG. 26C), and inguinal lymph nodes (LN) (FIG. 26D) are provided.

FIGS. 27A-27D provide flow cytometry plots showing splenic DC cell populations as a percentage of live CD45+ cells at 24 hours post-1^{st} dose (24h), 24 hours post-3^{rd} dose (6d) and 24 hours post-6^{th} dose (13d) in mice administered mRNAs encoding mOX40L, tethered mIL-12, and cell-associated hIL-15 or control mRNA (NST). The CD8+ cDC1 cell numbers (FIG. 27A), CD103+ cDC1 cell numbers (FIG. 27B), cDC2 cell numbers (FIG. 27C), and iDC cell numbers (FIG. 27D) are provided.

FIGS. 28A-28D provide flow cytometry plots showing inguinal lymph node DC cell populations as a percentage of live CD45+ cells at 24 hours post-1^{st} dose (24h), 24 hours post-3^{rd} dose (6d) and 24 hours post-6^{th} dose (13d) in mice administered mRNAs encoding mOX40L, tethered mIL-12, and cell-associated hIL-15 or control mRNA (NST). The CD8+cDC1 cell numbers (FIG. 28A), CD103+ cDC1 cell numbers (FIG. 28B), cDC2 cell numbers (FIG. 28C), and iDC cell numbers (FIG. 28D) are provided.

FIGS. 29A-29D provide flow cytometry plots showing expression of maturation marker, CD86, on splenic and inguinal lymph node CD8+ cDC1 and CD103+ cDC1 cell populations at 24 hours post-1^{st} dose (24 h), 24 hours post-3^{rd} dose (6d) and 24 hours post-6^{th} dose (13d) in mice administered mRNAs encoding mOX40L, tethered mIL-12, and cell-associated hIL-15 or control mRNA (NST). The CD86 MFI of splenic CD8+ cDC1 cells (FIG. 29A), splenic CD103+ cDC1 cells (FIG. 29B), inguinal lymph node CD8+ cDC1 cells (FIG. 29C), and inguinal lymph node CD103+ cDC1 cells (FIG. 29D) are provided.

FIGS. 30A-30D provide flow cytometry plots showing expression of maturation marker, CD86, on splenic and inguinal lymph node cDC2 and iDC cell populations at 24 hours post-Pt dose (24 h), 24 hours post-3^{rd} dose (6d) and 24 hours post-6^{th} dose (13d) in mice administered mRNAs encoding mOX40L, tethered mIL-12, and cell-associated hIL-15 or control mRNA (NST). The CD86 MFI of splenic cDC2 cells (FIG. 30A), splenic iDC cells (FIG. 30B), inguinal lymph node cDC2 cells (FIG. 30C), and inguinal lymph node iDC cells (FIG. 30D) are provided.

FIG. 31 provides a graph showing percent survival of C57BL/6 or Batf3 KO mice bearing AML. The tumor-bearing C57BL/6 were untreated, administered control mRNA (NST) or mRNAs encoding mOX40L, tethered mIL-12, and cell-associated hIL-15. The tumor-bearing Batf3 KO mice were administered 0.22 mg/kg three times a week for two weeks (TIW×3) of control mRNA (NST) or mRNAs encoding mOX40L, tethered mIL-12, and cell-associated hIL-15.

FIG. 32 provides a graph showing percent survival of mice administered control mRNA or mRNAs encoding mOX40L, tethered mIL-12, and cell-associated hIL-15 and left untreated, treated with isotype mAb, or treated with anti-CD4+mAb.

FIG. 33 provides a graph showing percent survival of mice administered control mRNA or mRNAs encoding mOX40L, tethered mIL-12, and cell-associated hIL-15 and left untreated, treated with isotype mAb, or treated with anti-IFNγ mAb.

FIGS. 34A-34B provide flow cytometry plots showing MHC II expression on monocytes, either as percent of monocytes (FIG. 34A) or MFI (FIG. 34B) after tumor bearing-mice were administered mRNAs encoding mOX40L, tethered mIL-12, and cell-associated hIL-15 or control mRNA, and further left untreated, treated with isotype mAb, or treated with anti-IFNγ mAb.

FIGS. 35A-35B provide flow cytometry plots showing PD-L¹ expression on myeloid cells, either as percent of myeloid cells (FIG. 35A) or MFI (FIG. 35B) after tumor bearing-mice were administered mRNAs encoding mOX40L, tethered mIL-12, and cell-associated hIL-15 or control mRNA, and further left untreated, treated with isotype mAb, or treated with anti-IFNγ mAb.

FIGS. 36A-36B provide flow cytometry plots showing PD-L¹ expression on granulocytes, either as percent of granulocytes (FIG. 36A) or MFI (FIG. 36B) after tumor bearing-mice were administered mRNAs encoding mOX40L, tethered mIL-12, and cell-associated hIL-15 or control mRNA, and further left untreated, treated with isotype mAb, or treated with anti-IFNγ mAb.

FIGS. 37A-37B provide flow cytometry plots showing PD-L¹ expression on monocytes, either as percent of monocytes (FIG. 37A) or MFI (FIG. 37B) after tumor bearing-mice were administered mRNAs encoding hOX40L, tethered hIL-12, and cell-associated hIL-15 or control mRNA, and further left untreated, treated with isotype mAb, or treated with anti-IFNγ mAb.

FIGS. 38A-38F provide graphs showing expression of IFNγ (FIGS. 38A-38C) and IP-10 (CXCL¹⁰) (FIGS. 38D-38F) in cynomolgus macaques after administration with mRNAs encoding hOX40L, tethered hIL-12, and cell-associated hIL-15 formulated in LNP.

FIGS. 39A-39C provides flow cytometry plots showing NK, NKT and CD8+ T cell numbers, respectively, (as percentage of live CD45+ cells) in spleen and bone marrow samples of macaques administered mRNAs encoding hOX40L, tethered hIL-12, and cell-associated hIL-15 or given Tris/Sucrose control injections.

FIGS. 40A-40D provides flow cytometry plots showing the percentage of CD8+ T cells, NK, CD4+ T cells and NKT cells, respectively, having the activation marker, CD69, in spleen and bone marrow samples of macaques administered hOX40L, tethered hIL-12, and cell-associated hIL-15 or given Tris/Sucrose control injections.

### DETAILED DESCRIPTION

A particularly exciting approach to treating cancer involves the prevention or treatment of disease with substances that stimulate the immune response, known as immunotherapy. Immunotherapy, also referred to in the art as immuno-oncology, has begun to revolutionize cancer treatment, by introducing therapies that target not the tumor, but the host immune system. These therapies possess unique pharmacological response profiles, and thus represent therapies that might cure many distinct types of cancer. Cancers of the lungs, kidney, bladder and skin are among those that derive substantial efficacy from treatment with immuno-oncology in terms of survival or tumor response, with melanoma possibly showing the greatest benefits.

Disseminated cancers are a significant health problem and are not effectively treated by conventional therapies. In particular, disseminated cancers, including metastatic cancers and cancers of the blood which do not ordinarily form solid tumors, such as myeloid malignancies (e.g., AML), are known to evade immune responses through a variety of mechanisms, thereby hindering the development of an effective immune response. For example, AML is known to evade NK cell lysis by upregulating NK inhibitor proteins, by suppressing NK activating ligands and/or by inducing NK cell anergy. Additionally, there is a low incidence of somatic mutations in AML, which leads to a low neo-antigen spectrum, thus resulting in a low AML-specific T cell response and low anti-tumor immunity (see e.g., Grove and Vassilou (2014) Dis. Models Mech. 7:94).

Although solid tumors can be treated by conventional therapies (e.g., surgery), numerous cancers are unresponsive to such therapies or relapse occurs. Moreover, the tumor microenvironment is complex and often dictates the outcome of therapeutic treatment.

Accordingly, methods and compositions useful for treating cancer and, in particular, methods and compositions which enhance immune responses (e.g., by NK cells and/or T cells) against cancer are of great interest.

Provided herein are compositions for use in treating cancer (e.g., solid tumors or disseminated cancers such as myeloid malignancies) comprising one or more polynucleotides (e.g., mRNAs, e.g., modified mRNAs) to stimulate particular immune cell populations in a subject in need thereof.

In some embodiments, the disclosure provides compositions for use in treating cancer (e.g., solid tumors or disseminated cancers such as myeloid malignancies) comprising at least two mRNAs (e.g., modified mRNAs), wherein the at least two mRNAs encode a human OX40L polypeptide, a human IL-12 polypeptide operably linked to a membrane domain comprising a transmembrane domain, a human IL-15 polypeptide, a human IL-15Rα polypeptide and combinations thereof. In some embodiments, the composition comprises:

(a) an mRNA encoding a human OX40L polypeptide and an mRNA encoding a human IL-12 polypeptide operably linked to a membrane domain comprising a transmembrane domain;

(b) an mRNA encoding a human OX40L polypeptide, an mRNA encoding a human IL-15 polypeptide and an mRNA encoding a human IL-15Rα polypeptide;

(c) an mRNA encoding a human OX40L polypeptide and an mRNA encoding a human IL-15 polypeptide operably linked to a human IL-15Rα polypeptide;

(d) an mRNA encoding a human IL-12 polypeptide operably linked to a membrane domain comprising a transmembrane domain, an mRNA encoding a human IL-15 polypeptide and an mRNA encoding a human IL-15Rα polypeptide;

(e) an mRNA encoding a human IL-12 polypeptide operably linked to a membrane domain comprising a transmembrane domain and an mRNA encoding a human IL-15 polypeptide operably linked to a human IL-15Rα polypeptide;

(f) an mRNA encoding an OX40L polypeptide, an mRNA encoding a human IL-12 polypeptide operably linked to a membrane domain comprising a transmembrane domain, an mRNA encoding a human IL-15 polypeptide and an mRNA encoding a human IL-15Rα polypeptide; or

(g) an mRNA encoding a human OX40L polypeptide, an mRNA encoding a human IL-12 polypeptide operably linked to a membrane domain comprising a transmembrane domain and an mRNA encoding a human IL-15 polypeptide operably linked to a human IL-15Rα polypeptide.

In some embodiments, the mRNAs encoding human OX40L, tethered human IL-12 and cell-associated human IL-15 are co-formulated in the LNP at a weight (mass) ratio of 1:1:1. In some embodiments, the mRNAs encoding human OX40L, tethered human IL-12 and cell-associated human IL-15 are co-formulated at a weight (mass) ratio of 1:1:1, and wherein the mRNA encoding cell-associated human IL-15 is encoded by two mRNAs encoding human IL-15 and human IL-15Rα, and wherein the two mRNAs are co-formulated at a molar ratio of 1:1.

In some embodiments, the mRNA of the disclosure encodes a human OX40L polypeptide, which is a human OX40L polypeptide comprising a cytoplasmic domain of OX40L. In some embodiments, the mRNA encodes a human OX40L polypeptide comprising a transmembrane domain of OX40L. In some embodiments, the mRNA encodes a human OX40L polypeptide comprising an extracellular domain of OX40L and a transmembrane of OX40L. In some embodiments, the mRNA encodes a human OX40L polypeptide comprising an extracellular domain of OX40L and a cytoplasmic domain of OX40L. In some embodiments, the mRNA encodes a human OX40L polypeptide comprising an extracellular domain of OX40L, a transmembrane of OX40L, and a cytoplasmic domain of OX40L.

In some embodiments, the mRNA encodes a human IL-12 polypeptide which is a membrane-tethered form of a human IL-12 polypeptide. For example, in some embodiments an mRNA of the disclosure encodes a human IL-12 polypeptide operably linked to a membrane domain, wherein the membrane domain comprises a transmembrane domain. In some embodiments, the membrane domain comprises a transmembrane domain and an intracellular domain. In some embodiments, the transmembrane and intracellular domains are derived from the same polypeptide. In some embodiments, the transmembrane and intracellular domains are derived from different polypeptides.

In some embodiments, the mRNA of the disclosure encodes a human IL-15 polypeptide which is a soluble human IL-15 polypeptide. In some embodiments, the mRNA of the disclosure encodes a human IL-15 polypeptide operably linked to a human IL-15Rα polypeptide, thereby forming a membrane-tethered from (e.g., a complex) of IL-15/IL-15Rα upon expression of the mRNA in a cell. In some embodiments, the disclosure provides a first mRNA encoding a human IL-15 polypeptide and a second mRNA encoding a human IL-15Rα polypeptide, thereby providing a membrane-tethered form (e.g., a complex) of IL-15/IL-15Rα, encoded by separate mRNAs. In some embodiments, the mRNA of the disclosure encodes a human IL-15Rα polypeptide comprising a sushi domain, which has high affinity for IL-15. In some embodiments, the mRNA of the disclosure encodes a human IL-15Rα comprising a sushi domain, a transmembrane domain and an intracellular domain. In some embodiments, the transmembrane and intracellular domains are the human IL-15Rα transmembrane and intracellular domains. In some embodiments, the transmembrane and intracellular domains are heterologous to IL-15Rα.

mRNA Encoding OX40L Polypeptide

Human OX40L was first identified on the surface of human lymphocytes infected with human T-cell leukemia virus type-I (HTLV-I) by Tanaka et al. (Tanaka et al., International Journal of Cancer (1985), 36(5):549-55). OX40L is the ligand for OX40 (CD134). OX40L has also been designated CD252 (cluster of differentiation 252), tumor necrosis factor (ligand) superfamily, member 4, tax-transcriptionally activated glycoprotein 1, TXGP1, or gp34. Human OX40L is 183 amino acids in length and contains three domains: a cytoplasmic domain of amino acids 1-23; a transmembrane domain of amino acids 24-50, and an extracellular domain of amino acids 51-183.

In some embodiments, a composition or method of the disclosure comprises an mRNA encoding a mammalian OX40L polypeptide. In some embodiments, the mammalian OX40L polypeptide is a murine OX40L polypeptide. In some embodiments, the mammalian OX40L polypeptide is a human OX40L polypeptide. In some embodiments, the OX40L polypeptide comprises an amino acid sequence set forth in SEQ ID NOs: 1-3.

In some embodiments, the mRNA encoding a human OX40L polypeptide encodes a human OX40L polypeptide comprising an amino acid sequence at least 50%, at least 60%, at least 70%, at least 80%, at least 85%, at least 90%, at least 95%, at least 98%, at least 99%, or 100% identical to an amino acid sequence set forth in SEQ ID NOs: 1-3 or an amino acid sequence encoded by a nucleotide sequence set forth in SEQ ID NOs: 4-11, wherein the human OX40L polypeptide is capable of binding to an OX40 receptor. In some embodiments, the mRNA encoding a human OX40L polypeptide encodes a human OX40L polypeptide comprising an amino acid sequence at least 50%, at least 60%, at least 70%, at least 80%, at least 85%, at least 90%, at least 95%, at least 98%, at least 99%, or 100% identical to SEQ ID NO: 1 and is capable of binding to an OX40 receptor. In some embodiments, the mRNA encoding a human OX40L polypeptide encodes a human OX40L polypeptide that consists essentially of SEQ ID NO: 1 and is capable of binding to an OX40 receptor.

In certain embodiments, the mRNA encoding a human OX40L polypeptide encodes a human OX40L polypeptide comprising an amino acid sequence set forth in SEQ ID NOs: 1-3, optionally with one or more conservative substitutions, wherein the conservative substitutions do not significantly affect the binding activity of the OX40L polypeptide to its receptor, i.e., the OX40L polypeptide binds to the OX40 receptor after the substitutions. In some embodiments, the mRNA encoding a human OX40L polypeptide encodes a human OX40L polypeptide comprising an amino acid sequence having at least 80%, at least 85%, at least 90%, at least 95%, at least 99% or 100% identity to any one of the amino acid sequences set forth in SEQ ID NOs: 1-3.

In other embodiments, an mRNA encoding a human OX40L polypeptide comprises a nucleotide sequence at least 50%, at least 60%, at least 70%, at least 80%, at least 85%, at least 90%, at least 95%, at least 98%, at least 99%, or 100% identical to any one of the nucleic acid sequences set forth in SEQ ID NOs: 4-11. In some embodiments, the mRNA encoding a human OX40L polypeptide comprises an open reading frame comprising a nucleotide sequence selected from any one of SEQ ID NOs: 9-11. In some embodiments, the mRNA encoding a human OX40L polypeptide comprises an open reading frame comprising a nucleotide sequence having at least 80%, at least 85%, at least 90%, at least 95%, at least 99%, or 100% identity to a nucleotide sequence selected from any one of SEQ ID NOs: 9-11. In some embodiments, the mRNA encoding a human OX40L polypeptide comprises an open reading frame comprising the nucleotide sequence set forth in SEQ ID NO: 9. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 9. In some embodiments, the mRNA encoding a human OX40L polypeptide comprises an open reading frame comprising the nucleotide sequence set forth in SEQ ID NO: 10. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 10. In some embodiments, the mRNA encoding a human OX40L polypeptide comprises an open reading frame comprising the nucleotide sequence set forth in SEQ ID NO: 11. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 11.

In some embodiments, the mRNA useful for the methods and compositions described herein comprises an open reading frame encoding an extracellular domain of OX40L. In other embodiments, the mRNA comprises an open reading frame encoding a cytoplasmic domain of OX40L. In some embodiments, the mRNA comprises an open reading frame encoding a transmembrane domain of OX40L. In certain embodiments, the mRNA comprises an open reading frame encoding an extracellular domain of OX40L and a transmembrane domain of OX40L. In other embodiments, the mRNA comprises an open reading frame encoding an extracellular domain of OX40L and a cytoplasmic domain of OX40L. In yet other embodiments, the mRNA comprises an open reading frame encoding an extracellular domain of OX40L, a transmembrane of OX40L, and a cytoplasmic domain of OX40L.

A person of skill in the art would understand that in addition to the native signal sequences and propeptide sequences implicitly disclosed in SEQ ID NOs: 1-11 (sequences present in the precursor form and absent in the mature corresponding form) and non-native signal peptides, other signal sequences can be used. Accordingly, references to OX40L polypeptide or mRNA according to SEQ ID NOs: 1-11 encompass variants in which an alternative signal peptide (or encoding sequence) known in the art has been attached to said OX40L polypeptide (or mRNA). It is also understood that references to the sequences disclosed in SEQ ID NOs: 1-11 through the application are equally applicable and encompass orthologs and functional variants (for example polymorphic variants) and isoforms of those sequences known in the art at the time the application was filed.

mRNA Encoding Cell-Associated Cytokine

In some embodiments, the methods and compositions described herein utilize mRNA encoding a cell-associated cytokine. Cytokines are small secreted proteins released by cells that have a specific effect on the interactions and communications between cells. To minimize unwanted/off-target effects of soluble cytokines, and potential systemic toxicity, the present disclosure utilizes at least one mRNA encoding a cell-associated cytokine. A cell-associated cytokine is one that either naturally or by design is associated with a cell surface. For example, in some embodiments, a soluble/secreted cytokine is modified to include a transmembrane domain such that the soluble/secreted cytokine will attach to a cell surface. In some embodiments, by “anchoring” or “tethering” a cytokine to a cell surface, systemic effects generally observed with administration of soluble cytokines are reduced.

In some embodiments, a cell-associated cytokine activates T cells, NK cells, or both T cells and NK cells. Methods for measuring T cell and NK cell activation are known to those of skill in the art. For example, NK and T cell activation can be measured by analyzing surface expression of an activation marker (e.g., CD25 and CD69) on an NK cell or T cell by e.g., flow cytometry.

In some embodiments, a cytokine suitable as a cell-associated cytokine is an IL-12 family member. In some embodiments, the IL-12 family member is a polypeptide selected from the group consisting of IL-12, IL-23, IL-12p40 subunit, IL-23p19 subunit, IL-27, IL-35, and combinations thereof.

In some embodiments, a cytokine suitable as a cell-associated cytokine is IL-15 as described herein.

mRNA Encoding Tethered IL-12 Polypeptide

In some embodiments, the methods and compositions described herein utilize mRNA encoding an IL-12 polypeptide operably linked to a membrane domain comprising a transmembrane domain.

Interleukin-12 (IL-12) is a pro-inflammatory cytokine that plays an important role in innate and adaptive immunity Gately, M K et al., Annu Rev Immunol. 16: 495-521 (1998). IL-12 functions primarily as a 70 kDa heterodimeric protein consisting of two disulfide-linked p35 (IL-12A) and p40 (IL-12B) subunits. Due to its ability to activate both NK cells and cytotoxic T cells, IL-12 protein has been studied as a promising anti-cancer therapeutic since 1994. See Nastala, C. L. et al., J Immunol 153: 1697-1706 (1994).

Despite high expectations for IL-12 as a therapeutic, early clinical studies did not yield satisfactory results. Lasek W. et al., Cancer Immunol Immunother 63: 419-435, 424 (2014). Repeated administration of IL-12, in most patients, led to adaptive response and a progressive decline of IL-12-induced interferon gamma (IFNγ) levels in blood. Id. Moreover, while it was recognized that IL-12-induced anti-cancer activity is largely mediated by the secondary secretion of IFNγ, the concomitant induction of IFNγ along with other cytokines (e.g., TNF-α) or chemokines (IP-10 or MIG) by IL-12 caused severe toxicity. Id.

To reduce toxicity, membrane-anchored versions of IL-12 have been generated as IL-12 is naturally soluble. PCT Application No. PCT/US2018/033436 describes mRNA encoding tethered IL-12 and is herein incorporated by reference in its entirety.

Accordingly, in some embodiments the mRNA encoding a human IL-12 polypeptide encodes a tethered human IL-12 polypeptide, wherein human IL-12 is operably linked to a membrane domain.

In some embodiments, the IL-12 polypeptide is a murine IL-12 polypeptide. In some embodiments, the IL-12 polypeptide is a human IL-12 polypeptide. In some embodiments, the IL-12 polypeptide comprises an amino acid sequence set forth in SEQ ID NOs: 33, 35, 39 or 40.

In some embodiments, the IL-12 polypeptide comprises a single polypeptide chain comprising the IL-12B and IL-12A polypeptides fused directly or by a linker. In other embodiments, the IL-12 polypeptide comprises two polypeptides, the first polypeptide comprising IL-12B and the second polypeptide comprising IL-12A. In some embodiments, the disclosure provides an IL-12A polypeptide and an IL-12B polypeptide, wherein the IL-12A and IL-12B polypeptides are on the same chain or different chains.

As used in the present disclosure, the term “IL-12 polypeptide” refers to, e.g., an IL-12p40 subunit of IL-12 (i.e., IL-12B), an IL-12p35 subunit of IL-12 (i.e., IL-12A), or to a fusion protein comprising an IL-12p40 subunit polypeptide and an IL-12p35 subunit polypeptide, operably linked to a membrane domain comprising a transmembrane domain. In some aspects, the fusion protein comprises an IL-12B polypeptide selected from:

(i) the full-length IL-12B polypeptide (e.g., having the same or essentially the same length as wild-type IL-12B);

(ii) a functional fragment of the full-length IL-12B polypeptide (e.g., a truncated (e.g., deletion of carboxy, amino terminal, or internal regions) sequence shorter than an IL-12B wild-type; but still retaining IL-12B functional activity);

(iii) a variant thereof (e.g., full-length or truncated IL-12B proteins in which one or more amino acids have been replaced, e.g., variants that retain all or most of the IL-12B activity of the polypeptide with respect to the wild type IL-12B polypeptide (such as, e.g., V33I, V298F, or any other natural or artificial variants known in the art); or

(iv) a fusion protein comprising (i) a full-length IL-12B wild-type, a functional fragment or a variant thereof, and (ii) a heterologous protein;

and/or

an IL-12A polypeptide selected from:

(i) the full-length IL-12A polypeptide (e.g., having the same or essentially the same length as wild-type IL-12A);

(ii) a functional fragment of the full-length IL-12A polypeptide (e.g., a truncated (e.g., deletion of carboxy, amino terminal, or internal regions) sequence shorter than an IL-12A wild-type; but still retaining IL-12A functional activity);

(iii) a variant thereof (e.g., full-length or truncated IL-12A proteins in which one or more amino acids have been replaced, e.g., variants that retain all or most of the IL-12A activity of the polypeptide with respect to the wild type IL-12A polypeptide (such as natural or artificial variants known in the art); or

(iv) a fusion protein comprising (i) a full-length IL-12A wild-type, a functional fragment or a variant thereof, and (ii) a heterologous protein.

In some embodiments, the mRNA encoding a human IL-12 polypeptide encodes a human IL-12 polypeptide comprising an amino acid sequence at least 50%, at least 60%, at least 70%, at least 80%, at least 85%, at least 90%, at least 95%, at least 98%, at least 99%, or 100% identical to an amino acid sequence listed in SEQ ID NOs: 33, 35, 39 or 40 or an amino acid sequence encoded by a nucleotide sequence listed in SEQ ID NOs: 34, 36 or 46, wherein the human IL-12 polypeptide is capable of binding to an IL-12 receptor.

In certain embodiments, the IL-12 polypeptide encoded by an mRNA of the disclosure comprises an amino acid sequence listed in SEQ ID NOs: 33, 35, 39 or 40, with one or more conservative substitutions, wherein the conservative substitutions do not significantly affect the binding activity of the IL-12 polypeptide to its receptor, i.e., the IL-12 polypeptide binds to the IL-12 receptor after the substitutions.

In other embodiments, an mRNA encoding a human IL-12 polypeptide comprises a nucleotide sequence at least 50%, at least 60%, at least 70%, at least 80%, at least 85%, at least 90%, at least 95%, at least 98%, at least 99%, or 100% identical to a nucleic acid sequence listed in SEQ ID NOs: 34, 36 or 46.

A person of skill in the art would understand that in addition to the native signal sequences and propeptide sequences implicitly disclosed in SEQ ID NOs: 33-40 and 46 (sequences present in the precursor form and absent in the mature corresponding form) and non-native signal peptides, other signal sequences can be used. Accordingly, references to IL-12 polypeptide or mRNA according to SEQ ID NOs: 33-40 and 46 encompass variants in which an alternative signal peptide (or encoding sequence) known in the art has been attached to said IL-12 polypeptide (or mRNA). It is also understood that references to the sequences disclosed in SEQ ID NOs: 33-40 and 46 through the application are equally applicable and encompass orthologs and functional variants (for example polymorphic variants) and isoforms of those sequences known in the art at the time the application was filed.

In some embodiments, the tethered IL-12 polypeptides encoded by the mRNAs of the disclosure comprise a membrane domain that tethers (i.e., anchors) the IL-12 polypeptide to a cell membrane (e.g., a transmembrane domain). In some embodiments, the tethered IL-12 polypeptides comprise a transmembrane domain. In some embodiments, the tethered IL-12 polypeptides comprise a transmembrane domain, and optionally an intracellular domain. In some embodiments, the tethered IL-12 polypeptides comprise a transmembrane domain and an intracellular domain.

In some embodiments, the membrane domain is from an integral membrane protein.

Integral membrane proteins can include, for example, integral polytopic proteins that contain a single-pass or multi-pass transmembrane domain that tethers the protein to a cell surface, including domains with hydrophobic α-helical or β-barrel (i.e., (3-sheet) structures. The amino-terminus (i.e., N-terminus) of Type I integral membrane proteins is located in the extracellular space, while the carboxy-terminus (i.e., C-terminus) of Type II integral membrane proteins is located in the extracellular space.

In some embodiments, a tethered IL-12 polypeptide of the disclosure comprises a transmembrane domain from an integral polytopic protein. In some embodiments, a tethered IL-12 polypeptide of the disclosure comprises a transmembrane domain from a Type I integral membrane protein. In some embodiments, a tethered IL-12 polypeptide comprises a transmembrane domain from a Type II integral membrane protein.

In some embodiments, the transmembrane domain comprises an intracellular domain (i.e., a domain that is localized to the intracellular space of a cell, e.g., a domain that is localized to the cytoplasm of a cell). In some embodiments, an intracellular domain has been removed from the transmembrane domain. In some embodiments, the transmembrane domain comprises a membrane domain without an intracellular domain.

Integral membrane proteins can also include, for example, integral monotopic proteins that contain a membrane domain that does not span the entire cell membrane but that tethers the protein to a cell surface. In some embodiments, a tethered IL-12 polypeptide of the disclosure comprises a membrane domain from an integral monotopic protein.

In some embodiments, the membrane domain is derived from a Cluster of Differentiation (CD) protein, CD8, CD80, CD4, a receptor, Platelet-Derived Growth Factor Receptor (PDGF-R), Interleukin-6 Receptor (IL-6R), transferrin receptor, Tumor Necrosis Factor (TNF) receptor, erythropoietin (EPO) receptor, a T Cell Receptor (TCR), TCR β-chain, a Fc receptor, FcγRII, FcεRI, an interferon receptor, type I interferon receptor, a growth factor, Stem Cell Factor (SCF), TNF-α, B7-1, Asialoglycoprotein, c-erbB-2, ICAM-1, an immunoglobulin, an IgG, an IgM, a viral glycoprotein, rabies virus glycoprotein, respiratory syncytial virus glycoprotein G (RSVG), vesicular stomatis virus glycoprotein (VSVG), a viral hemagglutinin (HA), influenza HA, vaccinia virus HA, or any combination thereof.

In some embodiments, the membrane domain is selected from the group consisting of: a CD8 transmembrane domain, a PDGF-R transmembrane domain, a CD80 transmembrane domain, and any combination thereof.

Exemplary amino acid sequences of transmembrane domains are set forth in SEQ ID NOs: 41-43.

In some embodiments, a membrane domain comprises a transmembrane domain of T-cell surface glycoprotein CD8 alpha chain (also known as CD8A or T-lymphocyte differentiation antigen T8/Leu-2), e.g., a transmembrane of UniProtKB—P01732. In some embodiments, the mRNA encoding a tethered IL-12, comprises a nucleotide sequence encoding a CD8 transmembrane polypeptide. In some embodiments, the mRNA encoding a tethered IL-12, comprises a nucleotide sequence encoding a CD8 transmembrane polypeptide as set forth in SEQ ID NO: 41. In some embodiments, the mRNA encoding a tethered IL-12 comprising a CD8 transmembrane domain, comprises an open reading frame comprising the nucleotide sequence set forth in SEQ ID NO: 69. In some embodiments, the mRNA encoding a tethered IL-12 comprising a CD8 transmembrane domain, comprises an open reading frame comprising a nucleotide sequence having at least 80%, at least 85%, at least 90%, at least 95%, or at least 99% identity to the nucleotide sequence set forth in SEQ ID NO: 69.

In some embodiments, a membrane domain comprises a transmembrane domain of platelet-derived growth factor receptor beta (EC:2.7.10.1) (also known as PDGF-R-beta, PDGFR-beta, beta platelet-derived growth factor receptor, beta-type platelet-derived growth factor receptor, CD140 antigen-like family member B, platelet-derived growth factor receptor 1, PDGFR-1, or CD140b), e.g., a transmembrane domain of UniProtKB—P09619. In some embodiments, the mRNA encoding a tethered IL-12, comprises a nucleotide sequence encoding a PDGFR-beta transmembrane polypeptide. In some embodiments, the mRNA encoding a tethered IL-12, comprises a nucleotide sequence encoding a PDGFR-beta transmembrane polypeptide as set forth in SEQ ID NO: 42. In some embodiments, the mRNA encoding a tethered IL-12 comprising a PDGFR-beta transmembrane domain, comprises an open reading frame comprising the nucleotide sequence set forth in SEQ ID NO: 62. In some embodiments, the mRNA encoding a tethered IL-12 comprising a PDGFR-beta transmembrane domain, comprises an open reading frame comprising a nucleotide sequence having at least 80%, at least 85%, at least 90%, at least 95%, or at least 99% identity to the nucleotide sequence set forth in SEQ ID NO: 62.

In some embodiments, a membrane domain comprises a transmembrane domain of T-lymphocyte activation antigen CD80 (also known as activation B7-1 antigen, BB1, CTLA-4 counter-receptor B7.1, or B7), e.g., a transmembrane domain of UniProtKB—P33681. In some embodiments, the mRNA encoding a tethered IL-12, comprises a nucleotide sequence encoding a CD80 transmembrane polypeptide. In some embodiments, the mRNA encoding a tethered IL-12, comprises a nucleotide sequence encoding a CD80 transmembrane polypeptide as set forth in SEQ ID NO: 43. In some embodiments, the mRNA encoding a tethered IL-12 comprising a CD80 transmembrane domain, comprises an open reading frame comprising the nucleotide sequence set forth in SEQ ID NO: 70. In some embodiments, the mRNA encoding a tethered IL-12 comprising a CD80 transmembrane domain, comprises an open reading frame comprising a nucleotide sequence having at least 80%, at least 85%, at least 90%, at least 95%, or at least 99% identity to the nucleotide sequence set forth in SEQ ID NO: 70.

In some embodiments, the membrane domain in the tethered IL-12 polypeptide comprises an amino acid sequence at least about 50%, at least about 60%, at least about 70%, at least about 80%, at least about 90%, at least about 95%, at least about 99%, or about 100% identical to SEQ ID NO: 41, SEQ ID NO: 42, SEQ ID NO: 43, or any combination thereof.

In some embodiments, the membrane domain comprises a transmembrane domain and an intracellular domain. In some embodiments, an intracellular domain is any oligopeptide or polypeptide known to act as a transmission signal in a cell. In some embodiments, the membrane domain comprises an intracellular domain to stabilize the tethered IL-12 polypeptide.

Intracellular domains useful in the methods and compositions of the present disclosure include at least those derived from any of the polypeptides in which transmembrane domains are derived, as described supra. For example, suitable intracellular domains include, but are not limited to, an intracellular domain derived from CD80, PDGFR, or any combination thereof.

In some embodiments, a membrane domain comprises an intracellular domain of platelet-derived growth factor receptor beta (EC:2.7.10.1) (also known as PDGF-R-beta, PDGFR-beta, beta platelet-derived growth factor receptor, beta-type platelet-derived growth factor receptor, CD140 antigen-like family member B, platelet-derived growth factor receptor 1, PDGFR-1, or CD140b), e.g., an intracellular domain of UniProtKB—P09619. In some embodiments, the mRNA encoding a tethered IL-12, comprises a nucleotide sequence encoding a PDGFR-beta intracellular polypeptide. In some embodiments, the mRNA encoding a tethered IL-12, comprises a nucleotide sequence encoding a PDGFR-beta intracellular polypeptide as set forth in SEQ ID NO: 48.

In some embodiments, a membrane domain comprises a truncated intracellular domain of PDGFR-beta. In some embodiments, a truncated intracellular domain of PDGFR-beta stabilizes the tethered IL-12 polypeptide compared to the wild-type PDGFR-beta intracellular domain. In some embodiments, the mRNA encoding a tethered IL-12, comprises a nucleotide sequence encoding a truncated PDGFR-beta intracellular polypeptide. In some embodiments, the mRNA encoding a tethered IL-12, comprises a nucleotide sequence encoding a truncated PDGFR-beta intracellular polypeptide as set forth in SEQ ID NO: 49. In some embodiments, the mRNA encoding a tethered IL-12, comprises a nucleotide sequence encoding a truncated PDGFR-beta intracellular polypeptide as set forth in SEQ ID NO: 50. In some embodiments, the mRNA encoding a tethered IL-12 comprising a truncated PDGFR-beta intracellular domain, comprises an open reading frame comprising the nucleotide sequence set forth in SEQ ID NO: 63. In some embodiments, the mRNA encoding a tethered IL-12 comprising a truncated PDGFR-beta intracellular domain, comprises an open reading frame comprising a nucleotide sequence having at least 80%, at least 85%, at least 90%, at least 95%, or at least 99% identity to the nucleotide sequence set forth in SEQ ID NO: 63. In some embodiments, the mRNA encoding a tethered IL-12 comprising a truncated PDGFR-beta intracellular domain, comprises an open reading frame comprising the nucleotide sequence set forth in SEQ ID NO: 64. In some embodiments, the mRNA encoding a tethered IL-12 comprising a truncated PDGFR-beta intracellular domain, comprises an open reading frame comprising a nucleotide sequence having at least 80%, at least 85%, at least 90%, at least 95%, or at least 99% identity to the nucleotide sequence set forth in SEQ ID NO: 64.

In other embodiments, a membrane domain comprises an intracellular domain of T-lymphocyte activation antigen CD80 (also known as activation B7-1 antigen, BB1, CTLA-4 counter-receptor B7.1, or B7), e.g., an intracellular domain of UniProtKB—P33681. In some embodiments, the mRNA encoding a tethered IL-12, comprises a nucleotide sequence encoding a CD80 intracellular polypeptide. In some embodiments, the mRNA encoding a tethered IL-12, comprises a nucleotide sequence encoding a CD80 intracellular polypeptide as set forth in SEQ ID NO: 47. In some embodiments, the mRNA encoding a tethered IL-12 comprising a CD80 intracellular domain, comprises an open reading frame comprising the nucleotide sequence set forth in SEQ ID NO: 71. In some embodiments, the mRNA encoding a tethered IL-12 comprising a CD80 intracellular domain, comprises an open reading frame comprising a nucleotide sequence having at least 80%, at least 85%, at least 90%, at least 95%, or at least 99% identity to the nucleotide sequence set forth in SEQ ID NO: 71.

In some embodiments, the tethered IL-12 polypeptides described herein comprise a membrane domain comprising a transmembrane domain and an intracellular domain derived from the same polypeptide (i.e., homologous). In some embodiments, the tethered IL-12 polypeptides described herein comprise a membrane domain comprising a CD80 transmembrane domain and CD80 intracellular domain. In some embodiments, the tethered IL-12 polypeptide described herein comprise a membrane domain comprising a PDGFR-beta transmembrane domain and PDGFR-beta intracellular domain. In some embodiments, the tethered IL-12 polypeptides described herein comprise a membrane domain comprising a transmembrane domain and an intracellular domain derived from different polypeptides (i.e., heterologous) (e.g., a CD80 transmembrane domain and a PDGFR-beta intracellular domain; a CD8 transmembrane domain and a CD80 intracellular domain; a CD8 transmembrane domain and a PDGFR-beta transmembrane domain; or a PDGFR-beta transmembrane domain and a CD80 intracellular domain).

In some embodiments, the membrane domain (e.g., transmembrane domain, and optional intracellular domain) in the tethered IL-12 polypeptide is located C-terminal to any IL-12 amino acid sequence (i.e., any amino acid sequence of IL-12A, IL-12B, or both IL-12A and IL-12B when both are present in the tethered IL-12 polypeptide). The phrase “located C-terminal to” indicates location in a polypeptide with respect to other sequences in the polypeptide in relation to the C-terminus of the polypeptide. A membrane domain (e.g., transmembrane domain, and optional intracellular domain) that is “C-terminal to” any IL-12 amino acid sequences means that the membrane domain is located closer to the C-terminus of the tethered IL-12 polypeptide than any IL-12 amino acid sequences.

In some embodiments, the membrane domain (e.g., transmembrane domain, and optional intracellular domain) in the tethered IL-12 polypeptide is located N-terminal to the IL-12 polypeptide. A membrane domain that is “N-terminal to” any IL-12 amino acid sequences means that the membrane domain is located closer to the N-terminus of the tethered IL-12 polypeptide than any IL-12 amino acid sequences.

In some embodiments, the membrane domain (e.g., transmembrane domain, and optional intracellular domain) in the tethered IL-12 polypeptide is linked to the IL-12 polypeptide by a linker, which is referred to herein as a “membrane domain linker” or a “transmembrane domain linker” when the membrane domain is a transmembrane domain, and optionally an intracellular domain. Non-limiting examples of linkers are disclosed elsewhere herein. In some embodiments, the membrane domain in the tethered IL-12 polypeptide is fused directly to the IL-12 polypeptide.

In some embodiments, a tethered human IL-12 polypeptide comprising a human CD8 transmembrane domain encoded by an mRNA comprises the amino acid sequence set forth in SEQ ID NO: 53. In some embodiments, the mRNA encoding a tethered human IL-12 polypeptide comprising a human CD8 transmembrane domain, comprises an open reading frame comprising the nucleotide sequence set forth in SEQ ID NO: 52. In some embodiments, the mRNA encoding a tethered human IL-12 polypeptide comprising a human CD8 transmembrane domain, comprises an open reading frame comprising a nucleotide sequence having at least 80%, at least 85%, at least 90%, at least 95% or at least 99% identity to the nucleotide sequence set forth in SEQ ID NO: 52.

In some embodiments, a tethered human IL-12 polypeptide comprising a human CD8 transmembrane domain encoded by an mRNA comprises the amino acid sequence set forth in SEQ ID NO: 55. In some embodiments, the mRNA encoding a tethered human IL-12 polypeptide comprising a human CD8 transmembrane domain, comprises an open reading frame comprising the nucleotide sequence set forth in SEQ ID NO: 54. In some embodiments, the mRNA encoding a tethered human IL-12 polypeptide comprising a human CD8 transmembrane domain, comprises an open reading frame comprising a nucleotide sequence having at least 80%, at least 85%, at least 90%, at least 95% or at least 99% identity to the nucleotide sequence set forth in SEQ ID NO: 54.

In some embodiments, a tethered human IL-12 polypeptide comprising a human CD80 transmembrane domain and intracellular domain encoded by an mRNA comprises the amino acid sequence set forth in SEQ ID NO: 61. In some embodiments, the mRNA encoding a tethered human IL-12 polypeptide comprising a human CD80 transmembrane domain and intracellular domain, comprises an open reading frame comprising the nucleotide sequence set forth in any one of SEQ ID NOs: 56-60. In some embodiments, the mRNA encoding a tethered human IL-12 polypeptide comprising a human CD8 transmembrane domain, comprises an open reading frame comprising a nucleotide sequence having at least 80%, at least 85%, at least 90%, at least 95% or at least 99% identity to the nucleotide sequence selected from any one of SEQ ID NOs: 56-60.

mRNA Encoding Cell-Associated IL-15/IL-15Rα

IL-15 is a member of the 4α-helix bundle family of cytokines and plays an important role in the development of an effective immune response. Waldmann, T. A., Cancer Immunol. Res. 3: 219-227 (2015). IL-15 is essential for the proper development of NK cells and long-term maintenance of memory CD8+ T cells. The IL-15 gene encodes a 162 amino acid preprotein having a signal peptide of 48 amino acids, with the mature protein being 114 amino acids in length. Bamford, R. N., et al., Proc. Natl. Acad. Sci. USA 93: 2897-2902 (1996). See also, e.g., GenBank Accession Numbers NM_000585 for the Homo sapiens IL-15 transcript variant 3 mRNA sequence and NP_000576 for the corresponding IL-15 isoform 1 preproprotein.

IL-15 shares certain structural similarity to interleukin-2 (IL2). Like IL-2, IL-15 signals through the IL-2 receptor beta chain (CD122) and the common gamma chain (CD132). But, unlike IL-2, IL-15 cannot effectively bind CD122 and CD132 on its own. IL-15 must first bind to the IL-15 alpha receptor subunit (IL-15Rα). The IL-15Rα gene encodes a 267 amino acid preprotein having a signal peptide of 30 amino acids, with the mature protein being 237 amino acids in length. See, e.g., GenBank Accession Numbers NM_002189 for the Homo sapiens IL-15Rα transcript variant 1 mRNA and NP_002180 for the Homo sapiens IL-15Rα isoform 1 precursor amino acid sequence.

Human IL-15Rα is predominantly a transmembrane protein that binds to IL-15 on the surface of cells such as activated dendritic cells and monocytes. Waldmann, T. A., Cancer Immunol. Res. 3: 219-227 (2015). The membrane bound complex of IL-15/IL-15Rα then presents IL-15 in trans to CD122 and CD132 subunits. Accordingly, IL-15Rα is an essential component of IL-15 activity, such that IL-15 is a naturally cell-associated cytokine.

Therefore, in some embodiments, the disclosure provides an mRNA encoding a human IL-15 polypeptide and an mRNA encoding a human IL-15Rα polypeptide. In some embodiments, the disclosure provides an mRNA encoding a human IL-15 polypeptide operably linked to a human IL-15Rα polypeptide.

In some embodiments, the IL-15 polypeptide and/or IL-15Rα polypeptide is a variant, a peptide or a polypeptide containing a substitution, and insertion and/or an addition, a deletion and/or a covalent modification with respect to a wild-type IL-15 and/or IL-15Rα sequence. As referred herein, the term “IL-15 polypeptide” refers to the mature IL-15 polypeptide (i.e., without its signal peptide and propeptide). In one embodiment, the IL-15 polypeptide includes a signal peptide and/or propeptide.

The term “IL-15Rα polypeptide” as used herein includes at least a Sushi domain and a hinge region of a full-length human IL-15Rα polypeptide. In some embodiments, the sushi domain of a full-length human IL-15Rα polypeptide comprises the amino acid sequence set forth in SEQ ID NO: 129. In some embodiments, the IL-15Rα polypeptide comprises the extracellular domain of the full-length human IL-15Rα polypeptide. In some embodiments, the extracellular domain of the full-length human IL-15Rα polypeptide comprises the amino acid sequence set forth in SEQ ID NO: 130. In other embodiments, the IL-15Rα polypeptide comprises the transmembrane region and/or intracellular domain of the full-length human IL-15Rα polypeptide. In some embodiments, the transmembrane region and/or intracellular domain of the full-length human IL-15Rα polypeptide comprises the amino acid sequence set forth in SEQ ID NO: 131. In other embodiments, the IL-15Rα polypeptide comprises the transmembrane region and/or intracellular domain of a heterologous polypeptide. For example, any of the transmembrane and/or intracellular domains described herein can be utilized as heterologous transmembrane and/or intracellular domains of the IL-15Rα polypeptide.

In some embodiments, sequence tags or amino acids, can be added to the sequences encoded by the polynucleotides of the invention (e.g., at the N-terminal or C-terminal ends), e.g., for localization. In some embodiments, amino acid residues located at the carboxy, amino terminal, or internal regions of a polypeptide of the invention can optionally be deleted.

In some aspects, the disclosure provides an mRNA encoding a human IL-15 polypeptide. In other aspects, the disclosure provides an mRNA encoding a human IL-15Rα polypeptide. In some embodiments, the mRNA of the disclosure encodes a fusion protein comprising a human IL-15 polypeptide and a human IL-15Rα polypeptide comprising at least a Sushi domain, which are operably linked. In other embodiments, the mRNA encodes two polypeptide chains, the first chain comprising a human IL-15 polypeptide and the second chain comprising a human IL-15Rα polypeptide.

In some embodiments, the IL-15 polypeptide is selected from:

(i) the mature human IL-15 polypeptide (e.g., having the same or essentially the same length as wild-type human IL-15) with or without a signal peptide;

(ii) a functional fragment of the human IL-15 polypeptide (e.g., a truncated (e.g., deletion of carboxy, amino terminal, or internal regions) sequence shorter than an IL-15 wildtype; but still retaining IL-15 activity);

(iii) a variant thereof (e.g., full-length, mature, or truncated IL-15 proteins in which one or more amino acids have been replaced, e.g., variants that retain all or most of the IL-15 activity of the polypeptide with respect to the wild-type IL-15 polypeptide; and

(iv) a fusion protein comprising (a) a mature human IL-15 wild-type, a functional fragment or a variant thereof, with or without a signal peptide and (b) a heterologous protein; and/or

In some embodiments, the IL-15Rα polypeptide is selected from:

(i) the full-length human IL-15Rα polypeptide (e.g., having the same or essentially the same length as wild-type human IL-15Rα);

(ii) a functional fragment of the full-length human IL-15Rα polypeptide (e.g., a truncated (e.g., deletion of carboxy, amino terminal, or internal regions) sequence shorter than an IL-15Rα wild-type; but still retaining IL-15Rα activity);

(iii) a variant thereof (e.g., full-length or truncated IL-15Rα proteins in which one or more amino acids have been replaced, e.g., variants that retain all or most of the IL-15Rα activity of the polypeptide with respect to the wild-typeIL-15Rα polypeptide (such as natural or artificial variants known in the art); and

(iv) a fusion protein comprising (a) a full-length human IL-15Rα wild-type, a functional fragment or a variant thereof, and (b) a heterologous protein.

In certain embodiments, the mRNA encodes a mammalian IL-15 and/or IL-15Rα polypeptide, such as a non-human (e.g., primate) IL-15 and/or IL-15Rα polypeptide, a functional fragment or a variant thereof.

In some embodiments, the human IL-15 polypeptide comprises an amino acid sequence at least 50%, at least 60%, at least 70%, at least 80%, at least 85%, at least 90%, at least 95%, at least 98%, at least 99%, or 100% identical to an amino acid sequence listed in SEQ ID NOs: 15 and 17 or an amino acid sequence encoded by a nucleotide sequence listed in SEQ ID NOs: 16, 19, 20 and 122, wherein the human IL-15 polypeptide is capable of binding to a human IL-15 receptor.

In certain embodiments, the human IL-15 polypeptide encoded by an mRNA of the disclosure comprises an amino acid sequence listed in SEQ ID NOs: 15 and 17 with one or more conservative substitutions, wherein the conservative substitutions do not significantly affect the binding activity of the IL-15 polypeptide to its receptor, i.e., the IL-15 polypeptide binds to the IL-15 receptor after the substitutions.

In other embodiments, an mRNA encoding a human IL-15 polypeptide comprises a nucleotide sequence at least 50%, at least 60%, at least 70%, at least 80%, at least 85%, at least 90%, at least 95%, at least 98%, at least 99%, or 100% identical to a nucleic acid sequence listed in SEQ ID NOs: 16, 19, 20 and 122.

In some embodiments, the human IL-15Rα polypeptide comprises an amino acid sequence at least 50%, at least 60%, at least 70%, at least 80%, at least 85%, at least 90%, at least 95%, at least 98%, at least 99%, or 100% identical to an amino acid sequence listed in SEQ ID NO: 13 or an amino acid sequence encoded by a nucleotide sequence listed in SEQ ID NOs: 14, 21 and 22, wherein the human IL-15Rα polypeptide is capable of binding to a human IL-15 polypeptide.

In certain embodiments, the human IL-15Rα polypeptide encoded by an mRNA of the disclosure comprises an amino acid sequence listed in SEQ ID NOs: 14, 21 and 22, with one or more conservative substitutions, wherein the conservative substitutions do not significantly affect the binding activity of the IL-15Rα polypeptide to its ligand, i.e., the IL-15Rα polypeptide binds to IL-15 after the substitutions.

In other embodiments, an mRNA encoding a human IL-15Rα polypeptide comprises a nucleotide sequence at least 50%, at least 60%, at least 70%, at least 80%, at least 85%, at least 90%, at least 95%, at least 98%, at least 99%, or 100% identical to a nucleic acid sequence listed in SEQ ID NOs: 14, 21 and 22.

In some embodiments, an mRNA encodes a human IL-15/IL-15Rα fusion polypeptide comprising an amino acid sequence at least 50%, at least 60%, at least 70%, at least 80%, at least 85%, at least 90%, at least 95%, at least 98%, at least 99%, or 100% identical to an amino acid sequence listed in SEQ ID NO: 13 or an amino acid sequence encoded by a nucleotide sequence listed in SEQ ID NOs: 14, 21 and 22.

In certain embodiments, the IL-15/IL-15Rα fusion polypeptide encoded by an mRNA of the disclosure comprises an amino acid sequence listed in SEQ ID NOs: 23, 27 and 123, with one or more conservative substitutions, wherein the conservative substitutions do not significantly affect the binding activity of the IL-15 polypeptide to its receptor.

In other embodiments, an mRNA encoding an IL-15/IL-15Rα fusion polypeptide comprises a nucleotide sequence at least 50%, at least 60%, at least 70%, at least 80%, at least 85%, at least 90%, at least 95%, at least 98%, at least 99%, or 100% identical to a nucleic acid sequence listed in SEQ ID NOs: 24-26, 28-30 and 124-126.

### Compositions of Cytokines and Costimulatory Molecules

In some embodiments, the disclosure provides a composition (e.g., a lipid nanoparticle) comprising at least two mRNAs described herein. In some embodiments, the disclosure provides a composition (e.g., a lipid nanoparticle) comprising two mRNAs described herein. In some embodiments, the disclosure provides a composition (e.g., a lipid nanoparticle) comprising three mRNAs described herein. In some embodiments, the disclosure provides a composition (e.g., a lipid nanoparticle) comprising four mRNAs described herein.

In some embodiments the composition comprises (i) an mRNA encoding a human OX40L polypeptide; and (ii) an mRNA encoding a tethered human IL-12 polypeptide. In some embodiments, the composition comprises (i) an mRNA encoding a human OX40L polypeptide, wherein the human OX40L polypeptide comprises the amino acid sequence set forth in SEQ ID NO: 1; and (ii) an mRNA encoding a tethered human IL-12 polypeptide, wherein the human tethered IL-12 polypeptide comprises an amino acid sequence selected from the group consisting of SEQ ID NOs 53, 55, 61 and 66. In some embodiments, the composition comprises (i) an mRNA encoding a human OX40L polypeptide, wherein the human OX40L polypeptide comprises the amino acid sequence set forth in SEQ ID NO: 1; and (ii) an mRNA encoding a tethered human IL-12 polypeptide, wherein the human IL-12 polypeptide comprises the amino acid sequence set forth in SEQ ID NO: 61. In some embodiments, the composition comprises (i) an mRNA encoding a human OX40L polypeptide, wherein the mRNA comprises an open reading frame comprising a nucleotide sequence selected from the group consisting of SEQ ID NOs: 4, 6 and 9-11; and (ii) an mRNA encoding a tethered human IL-12 polypeptide, wherein the mRNA comprises an open reading frame comprising a nucleotide sequence selected from the group consisting of SEQ ID NOs: 52, 54, 56-60 and 67. In some embodiments, the composition comprises (i) an mRNA encoding a human OX40L polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 4, 6 and 9-11; and (ii) an mRNA encoding a tethered human IL-12 polypeptide, wherein the mRNA comprises an open reading frame comprising a nucleotide sequence selected from the group consisting of SEQ ID NOs: 52, 54, 56-60 and 67. In some embodiments, the composition comprises (i) an mRNA encoding a human OX40L polypeptide, wherein the mRNA comprises an open reading frame comprising the nucleotide sequence set forth in SEQ ID NO: 11; and (ii) an mRNA encoding a tethered human IL-12 polypeptide, wherein the mRNA comprises an open reading frame comprising the nucleotide sequence set forth in SEQ ID NO: 60. In some embodiments, the composition comprises (i) an mRNA encoding a human OX40L polypeptide, wherein the mRNA comprises an open reading frame comprising a nucleotide sequence having least 80%, 85%, 90%, 95%, or 99% identity to the nucleotide sequence set forth in SEQ ID NO: 11; and (ii) an mRNA encoding a tethered human IL-12 polypeptide, wherein the mRNA comprises an open reading frame comprising nucleotide sequence having least 80%, 85%, 90%, 95%, or 99% identity to the nucleotide sequence set forth in SEQ ID NO: 60. In some embodiments, the disclosure provides a combination of an mRNA encoding a human OX40L polypeptide and an mRNA encoding a tethered human IL-12 polypeptide, as described herein, wherein the two mRNAs are encapsulated in the same or different lipid nanoparticles. In some embodiments, the two mRNAs are encapsulated in the same lipid nanoparticle. In some embodiments, the two mRNAs are encapsulated in two different lipid nanoparticles.

In some embodiments the composition comprises (i) an mRNA encoding a human OX40L polypeptide; (ii) an mRNA encoding a human IL-15 polypeptide; and (iii) an mRNA encoding a human IL-15Rα polypeptide. In some embodiments the composition comprises (i) an mRNA encoding a human OX40L polypeptide, wherein the human OX40L polypeptide comprises the amino acid sequence set forth in SEQ ID NO: 1; (ii) an mRNA encoding a human IL-15 polypeptide, wherein the human IL-15 polypeptide comprises an amino acid sequence selected from the group consisting of SEQ ID NOs: 15 and 17; and (iii) an mRNA encoding a human IL-15Rα polypeptide, wherein the human IL-15Rα polypeptide comprises the amino acid sequence set forth in SEQID NO: 13. In some embodiments the composition comprises (i) an mRNA encoding a human OX40L polypeptide, wherein the human OX40L polypeptide comprises the amino acid sequence set forth in SEQ ID NO: 1; (ii) an mRNA encoding a human IL-15 polypeptide, wherein the human IL-15 polypeptide comprises the amino acid set forth in SEQ ID NO: 17; and (iii) an mRNA encoding a human IL-15Rαpolypeptide, wherein the human IL-15Rα polypeptide comprises the amino acid sequence set forth in SEQ ID NO: 13. In some embodiments the composition comprises (i) an mRNA encoding a human OX40L polypeptide, wherein the mRNA comprises an open reading frame comprising a nucleotide sequence selected from the group consisting of SEQ ID NOs: 4, 6 and 9-11; (ii) an mRNA encoding a human IL-15 polypeptide, wherein the mRNA comprises an open reading frame comprising a nucleotide sequence selected from the group consisting of SEQ ID NOs: 16, 19, 20 and 122; and (iii) an mRNA encoding a human IL-15Rα polypeptide, wherein the mRNA comprises an open reading frame comprising a nucleotide sequence selected from the group consisting of SEQ ID NOs: 14, 21 and 22. In some embodiments the composition comprises (i) an mRNA encoding a human OX40L polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 4, 6 and 9-11; (ii) an mRNA encoding a human IL-15 polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 16, 19, 20 and 122; and (iii) an mRNA encoding a human IL-15Rα polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 14, 21 and 22. In some embodiments the composition comprises (i) an mRNA encoding a human OX40L polypeptide, wherein the mRNA comprises an open reading frame comprising the nucleotide sequence set forth in SEQ ID NO: 11; (ii) an mRNA encoding a human IL-15 polypeptide, wherein the mRNA comprises an open reading frame comprising the nucleotide sequence set forth in SEQ ID NO: 122; and (iii) an mRNA encoding a human IL-15Rα polypeptide, wherein the mRNA comprises an open reading frame comprising the nucleotide sequence set forth in SEQ ID NO: 22. In some embodiments the composition comprises (i) an mRNA encoding a human OX40L polypeptide, wherein the mRNA comprises an open reading frame comprising a nucleotide sequence having least 80%, 85%, 90%, 95%, or 99% identity to the nucleotide sequence set forth in SEQ ID NO: 11; (ii) an mRNA encoding a human IL-15 polypeptide, wherein the mRNA comprises an open reading frame comprising a nucleotide sequence having least 80%, 85%, 90%, 95%, or 99% identity to the nucleotide sequence set forth in SEQ ID NO: 122; and (iii) an mRNA encoding a human IL-15Rα polypeptide, wherein the mRNA comprises an open reading frame comprising a nucleotide sequence having least 80%, 85%, 90%, 95%, or 99% identity to the nucleotide sequence set forth in SEQ ID NO: 22. In some embodiments, the disclosure provides a combination of an mRNA encoding a human OX40L polypeptide, an mRNA encoding a human IL-15 polypeptide and an mRNA encoding a human IL-15Rα polypeptide, as described herein, wherein the three mRNAs are encapsulated in the same or different lipid nanoparticles. In some embodiments, the three mRNAs are encapsulated in the same lipid nanoparticle. In some embodiments, the three mRNAs are encapsulated in three different lipid nanoparticles.

In some embodiments the composition comprises (i) an mRNA encoding a human OX40L polypeptide; and (ii) an mRNA encoding a human IL-15 polypeptide operably linked to a human IL-15Rα polypeptide. In some embodiments the composition comprises (i) an mRNA encoding a human OX40L polypeptide, wherein the human OX40L polypeptide comprises the amino acid sequence set forth in SEQ ID NO: 1; and (ii) an mRNA encoding a human IL-15 polypeptide operably linked to a human IL-15Rα polypeptide comprising an amino acid sequence selected from the group consisting of SEQ ID NOs: 18, 23, 27 and 123. In some embodiments the composition comprises (i) an mRNA encoding a human OX40L polypeptide, wherein the mRNA comprises an open reading frame comprising a nucleotide sequence selected from the group consisting of SEQ ID NOs: 4, 6 and 9-11; and (ii) an mRNA encoding a human IL-15 polypeptide operably linked to a human IL-15Rα polypeptide, wherein the mRNA comprises an open reading frame comprising a nucleotide sequence selected from the group consisting of SEQ ID NOs: 24-26, 28-30 and 124-126. In some embodiments the composition comprises (i) an mRNA encoding a human OX40L polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 4, 6 and 9-11; and (ii) an mRNA encoding a human IL-15 polypeptide operably linked to a human IL-15Rα polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 24-26, 28-30 and 124-126. In some embodiments the composition comprises (i) an mRNA encoding a human OX40L polypeptide, wherein the mRNA comprises an open reading frame comprising the nucleotide sequence set forth in SEQ ID NO: 11; and (ii) an mRNA encoding a human IL-15 polypeptide operably linked to a human IL-15Rα polypeptide, wherein the mRNA comprises an open reading frame comprising a nucleotide sequence selected from the group consisting of SEQ ID NOs: 24-26, 28-30 and 124-126. In some embodiments the composition comprises (i) an mRNA encoding a human OX40L polypeptide, wherein the mRNA comprises an open reading frame comprising a nucleotide sequence having least 80%, 85%, 90%, 95%, or 99% identity to the nucleotide sequence set forth in SEQ ID NO: 11; and (ii) an mRNA encoding a human IL-15 polypeptide operably linked to a human IL-15Rα polypeptide, wherein the mRNA comprises an open reading frame a nucleotide sequence having at least 80%, 85%, 90%, 95%, or 99% identity to a nucleotide sequence selected from the group consisting of SEQ ID NOs: 24-26, 28-30 and 124-126. In some embodiments, the disclosure provides a combination of an mRNA encoding a human OX40L polypeptide and an mRNA encoding a human IL-15 polypeptide operably linked to a human IL-15Rα polypeptide, as described herein, wherein the two mRNAs are encapsulated in the same or different lipid nanoparticles. In some embodiments, the two mRNAs are encapsulated in the same lipid nanoparticle. In some embodiments, the two mRNAs are encapsulated in two different lipid nanoparticles.

In some embodiments the composition comprises (i) an mRNA encoding a tethered human IL-12 polypeptide; (ii) an mRNA encoding a human IL-15 polypeptide; and (iii) an mRNA encoding a human IL-15Rα polypeptide. In some embodiments the composition comprises (i) an mRNA encoding a tethered human IL-12 polypeptide, wherein the human IL-12 polypeptide comprises an amino acid sequence selected from the group consisting of SEQ ID NOs: 53, 55, 61 and 66; (ii) an mRNA encoding a human IL-15 polypeptide, wherein the human IL-15 polypeptide comprises an amino acid sequence selected from the group consisting of SEQ ID NOs: 15 and 17; and (iii) an mRNA encoding a human IL-15Rα polypeptide, wherein the human IL-15Rα polypeptide comprises the amino acid sequence set forth in SEQ ID NO: 13. In some embodiments the composition comprises (i) an mRNA encoding a tethered human IL-12 polypeptide, wherein the human IL-12 polypeptide comprises the amino acid sequence set forth in SEQ ID NO: 61; (ii) an mRNA encoding a human IL-15 polypeptide, wherein the human IL-15 polypeptide comprises the amino acid set forth in SEQ ID NO: 17; and (iii) an mRNA encoding a human IL-15Rα polypeptide, wherein the human IL-15Rα polypeptide comprises the amino acid sequence set forth in SEQ ID NO: 13. In some embodiments the composition comprises (i) an mRNA encoding a tethered human IL-12 polypeptide, wherein the mRNA comprises an open reading frame comprising a nucleotide sequence selected from the group consisting of SEQ ID NOs: 52, 54, 56-60 and 67; (ii) an mRNA encoding a human IL-15 polypeptide, wherein the mRNA comprises an open reading frame comprising a nucleotide sequence selected from the group consisting of SEQ ID NOs: 16, 19, 20 and 122; and (iii) an mRNA encoding a human IL-15Rα polypeptide, wherein the mRNA comprises an open reading frame comprising a nucleotide sequence selected from the group consisting of SEQ ID NOs: 14, 21 and 22. In some embodiments the composition comprises (i) an mRNA encoding a tethered human IL-12 polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 52, 54, 56-60 and 67; (ii) an mRNA encoding a human IL-15 polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 16, 19, 20 and 122; and (iii) an mRNA encoding a human IL-15Rα polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 14, 21 and 22. In some embodiments the composition comprises (i) an mRNA encoding a tethered human IL-12 polypeptide, wherein the mRNA comprises an open reading frame comprising the nucleotide sequence set forth in SEQ ID NO: 60; (ii) an mRNA encoding a human IL-15 polypeptide, wherein the mRNA comprises an open reading frame comprising the nucleotide sequence set forth in SEQ ID NO: 122; and (iii) an mRNA encoding a human IL-15Rα polypeptide, wherein the mRNA comprises an open reading frame comprising the nucleotide sequence set forth in SEQ ID NO: 22. In some embodiments the composition comprises (i) an mRNA encoding a tethered human IL-12 polypeptide, wherein the mRNA comprises an open reading frame comprising nucleotide sequence having least 80%, 85%, 90%, 95%, or 99% identity to the nucleotide sequence set forth in SEQ ID NO: 60; (ii) an mRNA encoding a human IL-15 polypeptide, wherein the mRNA comprises an open reading frame comprising a nucleotide sequence having least 80%, 85%, 90%, 95%, or 99% identity to the nucleotide sequence set forth in SEQ ID NO: 122; and (iii) an mRNA encoding a human IL-15Rα polypeptide, wherein the mRNA comprises an open reading frame comprising a nucleotide sequence having least 80%, 85%, 90%, 95%, or 99% identity to the nucleotide sequence set forth in SEQ ID NO: 22. In some embodiments, the disclosure provides a combination of an mRNA encoding a tethered human IL-12 polypeptide, an mRNA encoding a human IL-15 polypeptide and an mRNA encoding a human IL-15Rα polypeptide, as described herein, wherein the three mRNAs are encapsulated in the same or different lipid nanoparticles. In some embodiments, the three mRNAs are encapsulated in the same lipid nanoparticle. In some embodiments, the three mRNAs are encapsulated in three different lipid nanoparticles.

In some embodiments the composition comprises (i) an mRNA encoding a tethered human IL-12 polypeptide; and (ii) an mRNA encoding a human IL-15 polypeptide operably linked to a human IL-15Rα polypeptide. In some embodiments the composition comprises (i) an mRNA encoding a tethered human IL-12 polypeptide, wherein the human IL-12 polypeptide comprises an amino acid sequence selected from the group consisting of SEQ ID NOs: 53, 55, 61 and 66; and (ii) an mRNA encoding a human IL-15 polypeptide operably linked to a human IL-15Rα polypeptide comprising an amino acid sequence selected from the group consisting of SEQ ID NOs: 18, 23, 27 and 123. In some embodiments the composition comprises (i) an mRNA encoding a tethered human IL-12 polypeptide, wherein the human tethered IL-12 polypeptide comprises the amino acid sequence set forth in SEQ ID NO: 61; and (ii) an mRNA encoding a human IL-15 polypeptide operably linked to a human IL-15Rα polypeptide comprising an amino acid sequence selected from the group consisting of SEQ ID NOs: 18, 23, 27 and 123. In some embodiments the composition comprises (i) an mRNA encoding a tethered human IL-12 polypeptide, wherein the mRNA comprises an open reading frame comprising a nucleotide sequence selected from the group consisting of SEQ ID NOs: 52, 54, 56-60 and 67; and (ii) an mRNA encoding a human IL-15 polypeptide operably linked to a human IL-15Rα polypeptide, wherein the mRNA comprises an open reading frame comprising a nucleotide sequence selected from the group consisting of SEQ ID NOs: 24-26, 28-30 and 124-126. In some embodiments the composition comprises (i) an mRNA encoding a tethered human IL-12 polypeptide, wherein the mRNA comprises an open reading frame comprising a nucleotide sequence having least 80%, 85%, 90%, 95%, or 99% identity to a nucleotide sequence selected from the group consisting of SEQ ID NOs: 52, 54, 56-60 and 67; and (ii) an mRNA encoding a human IL-15 polypeptide operably linked to a human IL-15Rα polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 24-26, 28-30 and 124-126. In some embodiments the composition comprises (i) an mRNA encoding a tethered human IL-12 polypeptide, wherein the mRNA comprises an open reading frame comprising the nucleotide sequence set forth in SEQ ID NO: 60; and (ii) an mRNA encoding a human IL-15 polypeptide operably linked to a human IL-15Rα polypeptide, wherein the mRNA comprises an open reading frame comprising a nucleotide sequence selected from the group consisting of SEQ ID NOs: 24-26, 28-30 and 124-126. In some embodiments the composition comprises (i) an mRNA encoding a tethered human IL-12 polypeptide, wherein the mRNA comprises an open reading frame comprising a nucleotide sequence having least 80%, 85%, 90%, 95%, or 99% identity to the nucleotide sequence set forth in SEQ ID NO: 60; and (ii) an mRNA encoding a human IL-15 polypeptide operably linked to a human IL-15Rα polypeptide, wherein the mRNA comprises an open reading frame a nucleotide sequence having at least 80%, 85%, 90%, 95%, or 99% identity to a nucleotide sequence selected from the group consisting of SEQ ID NOs: 24-26, 28-30 and 124-126. In some embodiments, the disclosure provides a combination of an mRNA encoding a tethered human IL-12 polypeptide and an mRNA encoding a human IL-15 polypeptide operably linked to a human IL-15Rα polypeptide, as described herein, wherein the two mRNAs are encapsulated in the same or different lipid nanoparticles. In some embodiments, the two mRNAs are encapsulated in the same lipid nanoparticle. In some embodiments, the two mRNAs are encapsulated in two different lipid nanoparticles.

In some embodiments the composition comprises (i) an mRNA encoding a human OX40L polypeptide; (ii) an mRNA encoding a tethered human IL-12 polypeptide; (iii) an mRNA encoding a human IL-15 polypeptide; and (iv) an mRNA encoding a human IL-15Rα polypeptide. In some embodiments the composition comprises (i) an mRNA encoding a human OX40L polypeptide, wherein the human OX40L polypeptide comprises the amino acid sequence set forth in SEQ ID NO: 1; (ii) an mRNA encoding a tethered human IL-12 polypeptide, wherein the human IL-12 polypeptide comprises an amino acid sequence selected from the group consisting of SEQ ID NOs: 53, 55, 61 and 66; (iii) an mRNA encoding a human IL-15 polypeptide, wherein the human IL-15 polypeptide comprises an amino acid sequence selected from the group consisting of SEQ ID NOs: 15 and 17; and (iv) an mRNA encoding a human IL-15Rα polypeptide, wherein the human IL-15Rα polypeptide comprises the amino acid sequence set forth in SEQ ID NO: 13. In some embodiments the composition comprises (i) an mRNA encoding a human OX40L polypeptide, wherein the human OX40L polypeptide comprises the amino acid sequence set forth in SEQ ID NO: 1; (ii) an mRNA encoding a tethered human IL-12 polypeptide, wherein the human IL-12 polypeptide comprises the amino acid sequence set forth in SEQ ID NO: 61; (iii) an mRNA encoding a human IL-15 polypeptide, wherein the human IL-15 polypeptide comprises the amino acid set forth in SEQ ID NO: 17; and (iv) an mRNA encoding a human IL-15Rα polypeptide, wherein the human IL-15Rα polypeptide comprises the amino acid sequence set forth in SEQ ID NO: 13. In some embodiments the composition comprises (i) an mRNA encoding a human OX40L polypeptide, wherein the mRNA comprises an open reading frame comprising a nucleotide sequence selected from the group consisting of SEQ ID NOs: 4, 6 and 9-11; (ii) an mRNA encoding a tethered human IL-12 polypeptide, wherein the mRNA comprises an open reading frame comprising a nucleotide sequence selected from the group consisting of SEQ ID NOs: 52, 54, 56-60 and 67; (iii) an mRNA encoding a human IL-15 polypeptide, wherein the mRNA comprises an open reading frame comprising a nucleotide sequence selected from the group consisting of SEQ ID NOs: 16, 19, 20 and 122; and (iv) an mRNA encoding a human IL-15Rα polypeptide, wherein the mRNA comprises an open reading frame comprising a nucleotide sequence selected from the group consisting of SEQ ID NOs: 14, 21 and 22. In some embodiments the composition comprises (i) an mRNA encoding a human OX40L polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 4, 6 and 9-11; (ii) an mRNA encoding a tethered human IL-12 polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 52, 54, 56-60 and 67; (iii) an mRNA encoding a human IL-15 polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 16, 19, 20 and 122; and (iv) an mRNA encoding a human IL-15Rα polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 14, 21 and 22. In some embodiments the composition comprises (i) an mRNA encoding a human OX40L polypeptide, wherein the mRNA comprises an open reading frame comprising the nucleotide sequence set forth in SEQ ID NO: 11; (ii) an mRNA encoding a tethered human IL-12 polypeptide, wherein the mRNA comprises an open reading frame comprising the nucleotide sequence set forth in SEQ ID NO: 60; (iii) an mRNA encoding a human IL-15 polypeptide, wherein the mRNA comprises an open reading frame comprising the nucleotide sequence set forth in SEQ ID NO: 122; and (iv) an mRNA encoding a human IL-15Rα polypeptide, wherein the mRNA comprises an open reading frame comprising the nucleotide sequence set forth in SEQ ID NO: 22. In some embodiments the composition comprises (i) an mRNA encoding a human OX40L polypeptide, wherein the mRNA comprises an open reading frame comprising a nucleotide sequence having least 80%, 85%, 90%, 95%, or 99% identity to the nucleotide sequence set forth in SEQ ID NO: 11; (ii) an mRNA encoding a tethered human IL-12 polypeptide, wherein the mRNA comprises an open reading frame comprising nucleotide sequence having least 80%, 85%, 90%, 95%, or 99% identity to the nucleotide sequence set forth in SEQ ID NO: 60; (iii) an mRNA encoding a human IL-15 polypeptide, wherein the mRNA comprises an open reading frame comprising a nucleotide sequence having least 80%, 85%, 90%, 95%, or 99% identity to the nucleotide sequence set forth in SEQ ID NO: 122; and (iv) an mRNA encoding a human IL-15Rα polypeptide, wherein the mRNA comprises an open reading frame comprising a nucleotide sequence having least 80%, 85%, 90%, 95%, or 99% identity to the nucleotide sequence set forth in SEQ ID NO: 22 selected from the group consisting of SEQ ID NOs: 24-26, 28-30 and 124-126. In some embodiments, the disclosure provides a combination of an mRNA encoding a human OX40L polypeptide, an mRNA encoding a tethered human IL-12 polypeptide, an mRNA encoding a human IL-15 polypeptide and an mRNA encoding a human IL-15Rα polypeptide, as described herein, wherein the four mRNAs are encapsulated in the same or different lipid nanoparticles. In some embodiments, the four mRNAs are encapsulated in the same lipid nanoparticle. In some embodiments, the four mRNAs are encapsulated in three different lipid nanoparticles.

In some embodiments the composition comprises (i) an mRNA encoding a human OX40L polypeptide; (ii) an mRNA encoding a tethered human IL-12 polypeptide; and (iii) an mRNA encoding a human IL-15 polypeptide operably linked to a human IL-15Rα polypeptide. In some embodiments the composition comprises (i) an mRNA encoding a human OX40L polypeptide, wherein the human OX40L polypeptide comprises the amino acid sequence set forth in SEQ ID NO: 1; (ii) an mRNA encoding a tethered human IL-12 polypeptide, wherein the human IL-12 polypeptide comprises an amino acid sequence selected from the group consisting of SEQ ID NOs: 53, 55, 61 and 66; and (iii) an mRNA encoding a human IL-15 polypeptide operably linked to a human IL-15Rα polypeptide comprising an amino acid sequence selected from the group consisting of SEQ ID NOs: 18, 23, 27 and 123. In some embodiments the composition comprises (i) an mRNA encoding a human OX40L polypeptide, wherein the human OX40L polypeptide comprises the amino acid sequence set forth in SEQ ID NO: 1; (ii) an mRNA encoding a tethered human IL-12 polypeptide, wherein the human IL-12 polypeptide comprises the amino acid sequence set forth in SEQ ID NO: 61; and (iii) an mRNA encoding a human IL-15 polypeptide operably linked to a human IL-15Rα polypeptide comprising an amino acid sequence selected from the group consisting of SEQ ID NOs: 18, 23, 27 and 123. In some embodiments the composition comprises (i) an mRNA encoding a human OX40L polypeptide, wherein the mRNA comprises an open reading frame comprising a nucleotide sequence selected from the group consisting of SEQ ID NOs: 4, 6 and 9-11; (ii) an mRNA encoding a tethered human IL-12 polypeptide, wherein the mRNA comprises an open reading frame comprising a nucleotide sequence selected from the group consisting of SEQ ID NOs: 52, 54, 56-60 and 67; and (iii) an mRNA encoding a human IL-15 polypeptide operably linked to a human IL-15Rα polypeptide, wherein the mRNA comprises an open reading frame comprising a nucleotide sequence selected from the group consisting of SEQ ID NOs: 24-26, 28-30 and 124-126. In some embodiments the composition comprises (i) an mRNA encoding a human OX40L polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 4, 6 and 9-11; (ii) an mRNA encoding a tethered human IL-12 polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 52, 54, 56-60 and 67; and (iii) an mRNA encoding a human IL-15 polypeptide operably linked to a human IL-15Rα polypeptide, wherein the mRNA comprises an open reading frame comprising a nucleotide sequence having at least 80%, 85%, 90%, 95%, or 99% identity to a nucleotide sequence selected from the group consisting of SEQ ID NOs: 24-26, 28-30 and 124-126. In some embodiments the composition comprises (i) an mRNA encoding a human OX40L polypeptide, wherein the mRNA comprises an open reading frame comprising the nucleotide sequence set forth in SEQ ID NO: 11; (ii) an mRNA encoding a tethered human IL-12 polypeptide, wherein the mRNA comprises an open reading frame comprising the nucleotide sequence set forth in SEQ ID NO: 60; and (iii) an mRNA encoding a human IL-15 polypeptide operably linked to a human IL-15Rα polypeptide, wherein the mRNA comprises an open reading frame comprising a nucleotide sequence selected from the group consisting of SEQ ID NOs: 24-26, 28-30 and 124-126. In some embodiments the composition comprises (i) an mRNA encoding a human OX40L polypeptide, wherein the mRNA comprises an open reading frame comprising a nucleotide sequence having least 80%, 85%, 90%, 95%, or 99% identity to the nucleotide sequence set forth in SEQ ID NO: 11; (ii) an mRNA encoding a tethered human IL-12 polypeptide, wherein the mRNA comprises an open reading frame comprising nucleotide sequence having least 80%, 85%, 90%, 95%, or 99% identity to the nucleotide sequence set forth in SEQ ID NO: 60; and (iii) an mRNA encoding a human IL-15 polypeptide operably linked to a human IL-15Rα polypeptide, wherein the mRNA comprises an open reading frame a nucleotide sequence having at least 80%, 85%, 90%, 95%, or 99% identity to a nucleotide sequence selected from the group consisting of SEQ ID NOs: 24-26, 28-30 and 124-126. In some embodiments, the disclosure provides a combination of an mRNA encoding a human OX40L polypeptide, an mRNA encoding a tethered human IL-12 polypeptide and an mRNA encoding a human IL-15 polypeptide operably linked to a human IL-15Rα polypeptide, as described herein, wherein the three mRNAs are encapsulated in the same or different lipid nanoparticles. In some embodiments, the three mRNAs are encapsulated in the same lipid nanoparticle. In some embodiments, the three mRNAs are encapsulated in two different lipid nanoparticles.

mRNA Construct Components

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An exemplary 5′ UTR for use in the constructs is shown in SEQ ID NO: 75. Another exemplary 5′ UTR for use in the constructs is shown in SEQ ID NO: 76. Another exemplary 5′ UTR for use in the constructs is shown in SEQ ID NO: 133. Another exemplary 5′ UTR for use in the constructs is shown in SEQ ID NO: 12. An exemplary 3′ UTR for use in the constructs is shown in SEQ ID NO: 77. An exemplary 3′ UTR comprising miR-122 and miR-142.3p binding sites for use in the constructs is shown in SEQ ID NO: 78. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. A non-limiting list of possible cap species includes m⁷GpppG, m⁷Gpppm⁷G, m⁷3′dGpppG, m₂^{7,O3′}GpppG, m₂^{7,O3′}GppppG, m₂^{7,O2′}GppppG, m⁷Gpppm⁷G, m⁷3′dGpppG, m₂^{7,O3′}GpppG, m₂^{7,O3′}GppppG, and m₂^{7,O2′}, GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In one embodiment, the polynucleotides of the present disclosure may include a sequence encoding a self-cleaving peptide. The self-cleaving peptide may be, but is not limited to, a 2A peptide. A variety of 2A peptides are known and available in the art and may be used, including e.g., the foot and mouth disease virus (FMDV) 2A peptide, the equine rhinitis A virus 2A peptide, the Thosea asigna virus 2A peptide, and the porcine teschovirus-1 2A peptide. 2A peptides are used by several viruses to generate two proteins from one transcript by ribosome-skipping, such that a normal peptide bond is impaired at the 2A peptide sequence, resulting in two discontinuous proteins being produced from one translation event. As a non-limiting example, the 2A peptide may have the protein sequence: GSGATNFSLLKQAGDVEENPGP (SEQ ID NO: 79), fragments or variants thereof. In one embodiment, the 2A peptide cleaves between the last glycine and last proline. As another non-limiting example, the polynucleotides of the present disclosure may include a polynucleotide sequence encoding the 2A peptide having the protein sequence GSGATNFSLLKQAGDVEENPGP (SEQ ID NO: 79) fragments or variants thereof. One example of a polynucleotide sequence encoding the 2A peptide is:

GGAAGCGGAGCTACTAACTTCAGCCTGCTGAAGCAGGCTGGAGACGTGGAGGAG AACCCTGGACCT (SEQ ID NO: 80). In one illustrative embodiment, a 2A peptide is encoded by the following sequence: 5′-TCCGGACTCAGATCCGGGGATCTCAAAATTGTCGCTCCTGTCAAACAAACTCTTA ACTTTGATTTACTCAAACTGGCTGGGGATGTAGAAAGCAATCCAGGTCCACTC-3′ (SEQ ID NO: 81). The polynucleotide sequence of the 2A peptide may be modified or codon optimized by the methods described herein and/or are known in the art.

In one embodiment, this sequence may be used to separate the coding regions of two or more polypeptides of interest. As a non-limiting example, the sequence encoding the F2A peptide may be between a first coding region A and a second coding region B (A-F2Apep-B). The presence of the F2A peptide results in the cleavage of the one long protein between the glycine and the proline at the end of the F2A peptide sequence (NPGP is cleaved to result in NPG and P) thus creating separate protein A (with 21 amino acids of the F2A peptide attached, ending with NPG) and separate protein B (with 1 amino acid, P, of the F2A peptide attached). Likewise, for other 2A peptides (P2A, T2A and E2A), the presence of the peptide in a long protein results in cleavage between the glycine and proline at the end of the 2A peptide sequence (NPGP is cleaved to result in NPG and P). Protein A and protein B may be the same or different peptides or polypeptides of interest. In particular embodiments, protein A is a polypeptide that induces immunogenic cell death and protein B is another polypeptide that stimulates an inflammatory and/or immune response and/or regulates immune responsiveness (as described further below).

In some embodiments, the mRNA constructs described herein comprise a linker. In some embodiments, the linker is a peptide linker, including from one amino acid to about 200 amino acids. In some embodiments, the linker comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 amino acids.

In some embodiments, the linker can be GS (Gly/Ser) linkers, for example, comprising (GₙS)ₘ, wherein n is an integer from 1 to 20 and m is an integer from 1 to 20. In some embodiments, the GS linker can comprise (GGGGS)ₒ(SEQ ID NO: 86), wherein o is an integer from 1 to 5. In some embodiments, the GS linker can comprise GGSGGGGSGG (SEQ ID NO: 87), GGSGGGGG (SEQ ID NO: 88), or GSGSGSGS (SEQ ID NO: 89). In a particular embodiment, the linker is G₆S (GGGGGGS) (SEQ ID NO: 90).

In some embodiments, the linker can be a Gly-rich linker, for example, comprising (Gly)ₚ, wherein p is an integer from 1 to 40. In some embodiments, a Gly-rich linker can comprise GGGGG (SEQ ID NO: 91), GGGGGG (SEQ ID NO: 92), GGGGGGG (SEQ ID NO: 93) or GGGGGGGG (SEQ ID NO: 94).

In some embodiments, the linker can comprise (EAAAK)_{q} (SEQ ID NO: 95), wherein q is an integer from 1 to 5. In one embodiment, the linker can comprise (EAAAK)₃, i.e., EAAAKEAAAKEAAAK (SEQ ID NO: 96).

Further exemplary linkers include, but not limited to, GGGGSLVPRGSGGGGS (SEQ ID NO: 97), GSGSGS (SEQ ID NO: 98), GGGGSLVPRGSGGGG (SEQ ID NO: 99), GGSGGHMGSGG (SEQ ID NO: 100), GGSGGSGGSGG (SEQ ID NO: 101), GGSGG (SEQ ID NO: 102), GSGSGSGS (SEQ ID NO: 103), GGGSEGGGSEGGGSEGGG (SEQ ID NO: 104), AAGAATAA (SEQ ID NO: 105), GGSSG (SEQ ID NO: 106), GSGGGTGGGSG (SEQ ID NO: 107), GSGSGSGSGGSG (SEQ ID NO: 108), GSGGSGSGGSGGSG (SEQ ID NO: 109), and GSGGSGGSGGSGGS (SEQ ID NO: 110).

Nucleotides encoding the linkers disclosed herein can be constructed to fuse the ORF or ORFs of a polynucleotide disclosed herein.

Modified mRNAs

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′4)-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms² m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²2 G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m²′⁷Gm), 2′-O-methyl-inosine (Im), 1,2′-O-dimethyl-inosine (m¹Im), 2′-O-ribosylguanosine (phosphate) (Gr(p)) 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is 1-methyl-pseudouridine (m¹ψ), 5-methoxy-uridine (mo⁵U), 5-methyl-cytidine (m⁵C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases).

In some embodiments, the mRNA comprises pseudouridine (ψ). In some embodiments, the mRNA comprises pseudouridine (ψ) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (mo⁵U) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises N6-methyl-adenosine (m⁶A). In some embodiments, the mRNA comprises N6-methyl-adenosine (m⁶A) and 5-methyl-cytidine (m⁵C).

In certain embodiments, an mRNA of the disclosure is uniformly modified (i.e., fully modified, modified through-out the entire sequence) for a particular modification. In some embodiments, an mRNA of the disclosure is modified wherein at least 95%, at least 96%, at least 97%, at least 98%, at least 99% or 100% of a specified nucleotide or nucleobase is modified. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the disclosure can be uniformly modified for any type of nucleoside residue present in the sequence by replacement with a modified residue such as those set forth above. In some embodiments, an mRNA of the disclosure is uniformly modified with 1-methyl pseudouridine (m¹ψ), meaning that all uridine residues in the mRNA sequence are replaced with 1-methyl pseudouridine (m¹ψ). In some embodiments, at least 95%, at least 96%, at least 97%, at least 98%, at least 99% or 100% of uridines are 1-methyl pseudouridine (m¹ψ).

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

### Untranslated Regions (UTRs)

Translation of a polynucleotide comprising an open reading frame encoding a polypeptide can be controlled and regulated by a variety of mechanisms that are provided by various cis-acting nucleic acid structures. For example, naturally-occurring, cis-acting RNA elements that form hairpins or other higher-order (e.g., pseudoknot) intramolecular mRNA secondary structures can provide a translational regulatory activity to a polynucleotide, wherein the RNA element influences or modulates the initiation of polynucleotide translation, particularly when the RNA element is positioned in the 5′ UTR close to the 5′-cap structure (Pelletier and Sonenberg (1985) Cell 40(3):515-526; Kozak (1986) Proc Natl Acad Sci 83:2850-2854).

Untranslated regions (UTRs) are nucleic acid sections of a polynucleotide before a start codon (5′ UTR) and after a stop codon (3′ UTR) that are not translated. In some embodiments, a polynucleotide (e.g., a ribonucleic acid (RNA), e.g., a messenger RNA (mRNA)) of the invention comprising an open reading frame (ORF) encoding a polypeptide further comprises UTR (e.g., a 5′ UTR or functional fragment thereof, a 3′ UTR or functional fragment thereof, or a combination thereof).

Cis-acting RNA elements can also affect translation elongation, being involved in numerous frameshifting events (Namy et al., (2004) Mol Cell 13(2):157-168). Internal ribosome entry sequences (IRES) represent another type of cis-acting RNA element that are typically located in 5′ UTRs, but have also been reported to be found within the coding region of naturally-occurring mRNAs (Holcik et al. (2000) Trends Genet 16(10):469-473). In cellular mRNAs, IRES often coexist with the 5′-cap structure and provide mRNAs with the functional capacity to be translated under conditions in which cap-dependent translation is compromised (Gebauer et al., (2012) Cold Spring Harb Perspect Biol 4(7):a012245). Another type of naturally-occurring cis-acting RNA element comprises upstream open reading frames (uORFs). Naturally-occurring uORFs occur singularly or multiply within the 5′ UTRs of numerous mRNAs and influence the translation of the downstream major ORF, usually negatively (with the notable exception of GCN4 mRNA in yeast and ATF4 mRNA in mammals, where uORFs serve to promote the translation of the downstream major ORF under conditions of increased eIF2 phosphorylation (Hinnebusch (2005) Annu Rev Microbiol 59:407-450)). Additional exemplary translational regulatory activities provided by components, structures, elements, motifs, and/or specific sequences comprising polynucleotides (e.g., mRNA) include, but are not limited to, mRNA stabilization or destabilization (Baker & Parker (2004) Curr Opin Cell Biol 16(3):293-299), translational activation (Villalba et al., (2011) Curr Opin Genet Dev 21(4):452-457), and translational repression (Blumer et al., (2002) Mech Dev 110(1-2):97-112). Studies have shown that naturally-occurring, cis-acting RNA elements can confer their respective functions when used to modify, by incorporation into, heterologous polynucleotides (Goldberg-Cohen et al., (2002) J Biol Chem 277(16):13635-13640).

### Functional RNA Elements

In some embodiments, the disclosure provides polynucleotides comprising a modification (e.g., an RNA element), wherein the modification provides a desired translational regulatory activity. Such modifications are described in PCT Application No. PCT/US2018/033519, herein incorporated by reference in its entirety.

In some embodiments, the disclosure provides a polynucleotide comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the desired translational regulatory activity is a cis-acting regulatory activity. In some embodiments, the desired translational regulatory activity is an increase in the residence time of the 43S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is a combination of one or more of the foregoing translational regulatory activities.

Accordingly, the present disclosure provides a polynucleotide, e.g., an mRNA, comprising an RNA element that comprises a sequence and/or an RNA secondary structure(s) that provides a desired translational regulatory activity as described herein. In some aspects, the mRNA comprises an RNA element that comprises a sequence and/or an RNA secondary structure(s) that promotes and/or enhances the translational fidelity of mRNA translation. In some aspects, the mRNA comprises an RNA element that comprises a sequence and/or an RNA secondary structure(s) that provides a desired translational regulatory activity, such as inhibiting and/or reducing leaky scanning. In some aspects, the disclosure provides an mRNA that comprises an RNA element that comprises a sequence and/or an RNA secondary structure(s) that inhibits and/or reduces leaky scanning thereby promoting the translational fidelity of the mRNA.

In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

In some embodiments, the disclosure provides an mRNA having one or more structural modifications that inhibits leaky scanning and/or promotes the translational fidelity of mRNA translation, wherein at least one of the structural modifications is a GC-rich RNA element. In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In some embodiments, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In some embodiments, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In some embodiments, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10, n=2 to 8, n=3 to 6, or n=4 to 5. In some embodiments, the sequence comprises a repeating GC-motif [CCG]n, wherein n=1, 2, 3, 4 or 5. In some embodiments, the sequence comprises a repeating GC-motif [CCG]n, wherein n=1, 2, or 3. In some embodiments, the sequence comprises a repeating GC-motif [CCG]n, wherein n=1. In some embodiments, the sequence comprises a repeating GC-motif [CCG]n, wherein n=2. In some embodiments, the sequence comprises a repeating GC-motif [CCG]n, wherein n=3. In some embodiments, the sequence comprises a repeating GC-motif [CCG]n, wherein n=4 (SEQ ID NO: 111). In some embodiments, the sequence comprises a repeating GC-motif [CCG]n, wherein n=5 (SEQ ID NO: 112).

In some embodiments, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In some embodiments, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element comprises any one of the sequences provided herein. In some embodiments, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In some embodiments, the GC-rich RNA element is located about 15-30, 15-20, 15-25, 10-15, or 5-10 nucleotides upstream of a Kozak consensus sequence. In some embodiments, the GC-rich RNA element is located immediately adjacent to a Kozak consensus sequence in the 5′ UTR of the mRNA.

In some embodiments, the disclosure provides an mRNA comprising at least one modification, wherein at least one modification is a GC-rich RNA element comprising the sequence set forth in SEQ ID NO: 113, or derivatives or analogs thereof, preceding a Kozak consensus sequence in the 5′ UTR of the mRNA. In some embodiments, the GC-rich element comprises the sequence as set forth in SEQ ID NO: 113 located immediately adjacent to and upstream of the Kozak consensus sequence in the 5′ UTR of the mRNA. In some embodiments, the GC-rich element comprises the sequence as set forth in SEQ ID NO: 113 located 1, 2, 3, 4, 5, 6, 7, 8, 9 or 10 bases upstream of the Kozak consensus sequence in the 5′ UTR of the mRNA. In other embodiments, the GC-rich element comprises the sequence as set forth in SEQ ID NO: 113 located 1-3, 3-5, 5-7, 7-9, 9-12, or 12-15 bases upstream of the Kozak consensus sequence in the 5′ UTR of the mRNA.

In some embodiments, the disclosure provides an mRNA comprising at least one modification, wherein at least one modification is a GC-rich RNA element comprising the sequence as set forth SEQ ID NO: 114, or derivatives or analogs thereof, preceding a Kozak consensus sequence in the 5′ UTR of the mRNA. In some embodiments, the GC-rich element comprises the sequence as set forth SEQ ID NO: 114 located immediately adjacent to and upstream of the Kozak consensus sequence in the 5′ UTR of the mRNA. In some embodiments, the GC-rich element comprises the sequence as set forth SEQ ID NO: 114 located 1, 2, 3, 4, 5, 6, 7, 8, 9 or 10 bases upstream of the Kozak consensus sequence in the 5′ UTR of the mRNA. In other embodiments, the GC-rich element comprises the sequence as set forth SEQ ID NO: 114 located 1-3, 3-5, 5-7, 7-9, 9-12, or 12-15 bases upstream of the Kozak consensus sequence in the 5′ UTR of the mRNA.

In some embodiments, the disclosure provides an mRNA comprising at least one modification, wherein at least one modification is a GC-rich RNA element comprising the sequence as set forth in SEQ ID NO: 115, or derivatives or analogs thereof, preceding a Kozak consensus sequence in the 5′ UTR of the mRNA. In some embodiments, the GC-rich element comprises the sequence as set forth in SEQ ID NO: 115 located immediately adjacent to and upstream of the Kozak consensus sequence in the 5′ UTR of the mRNA. In some embodiments, the GC-rich element comprises the sequence as set forth in SEQ ID NO: 115 located 1, 2, 3, 4, 5, 6, 7, 8, 9 or 10 bases upstream of the Kozak consensus sequence in the 5′ UTR of the mRNA. In other embodiments, the GC-rich element comprises the sequence as set forth in SEQ ID NO: 115 located 1-3, 3-5, 5-7, 7-9, 9-12, or 12-15 bases upstream of the Kozak consensus sequence in the 5′ UTR of the mRNA.

In some embodiments, the disclosure provides an mRNA comprising at least one modification, wherein at least one modification is a GC-rich RNA element comprising the sequence set forth in SEQ ID NO: 113, or derivatives or analogs thereof, preceding a Kozak consensus sequence in the 5′ UTR of the mRNA, wherein the 5′ UTR comprises the sequence set forth in SEQ ID NO: 116.

In some embodiments, the GC-rich element comprises the sequence set forth in SEQ ID NO: 113 located immediately adjacent to and upstream of the Kozak consensus sequence in a 5′ UTR sequence described herein. In some embodiments, the GC-rich element comprises the sequence set forth in SEQ ID NO: 113 located 1, 2, 3, 4, 5, 6, 7, 8, 9 or 10 bases upstream of the Kozak consensus sequence in the 5′ UTR of the mRNA, wherein the 5′ UTR comprises the sequence shown in SEQ ID NO: 116.

In other embodiments, the GC-rich element comprises the sequence set forth in SEQ ID NO: 113 located 1-3, 3-5, 5-7, 7-9, 9-12, or 12-15 bases upstream of the Kozak consensus sequence in the 5′ UTR of the mRNA, wherein the 5′ UTR comprises the sequence set forth in SEQ ID NO: 116.

In some embodiments, the 5′ UTR comprises the sequence set forth in SEQ ID NO: 117.

In some embodiments, the 5′ UTR comprises the sequence set forth in SEQ ID NO: 118.

In some embodiments, the disclosure provides an mRNA comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In one embodiment, the stable RNA secondary structure is upstream of the Kozak consensus sequence. In another embodiment, the stable RNA secondary structure is located about 30, about 25, about 20, about 15, about 10, or about 5 nucleotides upstream of the Kozak consensus sequence. In another embodiment, the stable RNA secondary structure is located about 20, about 15, about 10 or about 5 nucleotides upstream of the Kozak consensus sequence. In another embodiment, the stable RNA secondary structure is located about 5, about 4, about 3, about 2, about 1 nucleotides upstream of the Kozak consensus sequence. In another embodiment, the stable RNA secondary structure is located about 15-30, about 15-20, about 15-25, about 10-15, or about 5-10 nucleotides upstream of the Kozak consensus sequence. In another embodiment, the stable RNA secondary structure is located 12-15 nucleotides upstream of the Kozak consensus sequence. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to −10 kcal/mol.

In another embodiment, the modification is operably linked to an open reading frame encoding a polypeptide and wherein the modification and the open reading frame are heterologous.

In another embodiment, the sequence of the GC-rich RNA element is comprised exclusively of guanine (G) and cytosine (C) nucleobases.

RNA elements that provide a desired translational regulatory activity as described herein can be identified and characterized using known techniques, such as ribosome profiling. Ribosome profiling is a technique that allows the determination of the positions of PICs and/or ribosomes bound to mRNAs (see e.g., Ingolia et al., (2009) Science 324(5924):218-23, incorporated herein by reference). The technique is based on protecting a region or segment of mRNA, by the PIC and/or ribosome, from nuclease digestion. Protection results in the generation of a 30-bp fragment of RNA termed a ‘footprint’. The sequence and frequency of RNA footprints can be analyzed by methods known in the art (e.g., RNA-seq). The footprint is roughly centered on the A-site of the ribosome. If the PIC or ribosome dwells at a particular position or location along an mRNA, footprints generated at these positions would be relatively common. Studies have shown that more footprints are generated at positions where the PIC and/or ribosome exhibits decreased processivity and fewer footprints where the PIC and/or ribosome exhibits increased processivity (Gardin et al., (2014) eLife 3:e03735). In some embodiments, residence time or the time of occupancy of the PIC or ribosome at a discrete position or location along a polynucleotide comprising any one or more of the RNA elements described herein is determined by ribosome profiling.

A UTR can be homologous or heterologous to the coding region in a polynucleotide. In some embodiments, the UTR is homologous to the ORF encoding the polypeptide. In some embodiments, the UTR is heterologous to the ORF encoding the polypeptide. In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO:135), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML¹, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of: a globin, such as an α- or (3-globin (e.g., a Xenopus, mouse, rabbit, or human globin); a strong Kozak translational initiation signal; a CYBA (e.g., human cytochrome b-245 a polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a βglobin 5′ UTR; a 5′UTR containing a strong Kozak translational initiation signal; a cytochrome b-245 a polypeptide (CYBA) 5′ UTR; a hydroxysteroid (17-(3) dehydrogenase (HSD17B4) 5′ UTR; a Tobacco etch virus (TEV) 5′ UTR; a Venezuelan equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a βglobin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a 13 subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the invention comprise a 5′ UTR and/or a 3′ UTR selected from any of the UTRs disclosed herein. In some embodiments, the 5′ UTR comprises:

5′ UTR-001 (Upstream UTR)
(SEQ ID NO.: 76)
(GGGAAAUAAGAGAGAAAAGAAGAGUAAGAAGAAAUAUAAGAGCCACC);
5′ UTR-002 (Upstream UTR)
(SEQ ID NO.: 136
(GGGAGAUCAGAGAGAAAAGAAGAGUAAGAAGAAAUAUAAGAGCCACC);
5′ UTR-003 (Upstream UTR)
(See W02016/100812);
5′ UTR-004 (Upstream UTR)
(SEQ ID NO.: 137)
(GGGAGACAAGCUUGGCAUUCCGGUACUGUUGGUAAAGCCACC);
5′ UTR-006 (Upstream UTR)
(See W02016/100812);
5′ UTR-008 (Upstream UTR)
(SEQ ID NO.: 138)
(GGGAAUUAACAGAGAAAAGAAGAGUAAGAAGAAAUAUAAGAGCCACC);
5′ UTR-009 (Upstream UTR)
(SEQ ID NO.: 139)
(GGGAAAUUAGACAGAAAAGAAGAGUAAGAAGAAAUAUAAGAGCCACC);
5′ UTR-010, Upstream
(SEQ ID NO.: 140)
(GGGAAAUAAGAGAGUAAAGAACAGUAAGAAGAAAUAUAAGAGCCACC);
5′ UTR-011 (Upstream UTR)
(SEQ ID NO.: 141)
(GGGAAAAAAGAGAGAAAAGAAGACUAAGAAGAAAUAUAAGAGCCACC);
5′ UTR-012 (Upstream UTR)
(SEQ ID NO.: 142)
(GGGAAAUAAGAGAGAAAAGAAGAGUAAGAAGAUAUAUAAGAGCCACC);
5′ UTR-013 (Upstream UTR)
(SEQ ID NO.: 143)
(GGGAAAUAAGAGACAAAACAAGAGUAAGAAGAAAUAUAAGAGCCACC);
5′ UTR-014 (Upstream UTR)
(SEQ ID NO.: 144)
(GGGAAAUUAGAGAGUAAAGAACAGUAAGUAGAAUUAAAAGAGCCACC);
5′ UTR-015 (Upstream UTR)
(SEQ ID NO.: 145)
(GGGAAAUAAGAGAGAAUAGAAGAGUAAGAAGAAAUAUAAGAGCCACC);
5′ UTR-016 (Upstream UTR)
(SEQ ID NO.: 146)
(GGGAAAUAAGAGAGAAAAGAAGAGUAAGAAGAAAAUUAAGAGCCACC);
5′ UTR-017 (Upstream UTR);
or
(SEQ ID NO.: 147)
(GGGAAAUAAGAGAGAAAAGAAGAGUAAGAAGAAAUUUAAGAGCCACC);
5′ UTR-018 (Upstream UTR) 5′ UTR
(SEQ ID NO.: 75)
(UCAAGCUUUUGGACCCUCGUACAGAAGCUAAUACGACUCACUAUAGGG
AAAUAAGAGAGAAAAGAAGAGUAAGAAGAAAUAUAAGAGCCACC).

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of SEQ ID NOs: 75-76, 116-118, 132-134 or 136-147 and/or 3′ UTR sequences comprises any of SEQ ID NOs: 4, 77-78 or 121, and any combination thereof.

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of SEQ ID NO: 75, SEQ ID NO: 76, SEQ ID NO: 116, SEQ ID NO: 117, SEQ ID NO: 118, SEQ ID NO: 132 or SEQ ID NO:134 and/or 3′ UTR sequences comprises any of SEQ ID NO: 77, SEQ ID NO: 78, or SEQ ID NO: 121, and any combination thereof.

In some embodiments, the 5′ UTR comprises a nucleotide sequence set forth SEQ ID NO:75, SEQ ID NO:76, SEQ ID NO:116, SEQ ID NO: 117, SEQ ID NO: 118, SEQ ID NO: 132 or SEQ ID NO:134. In some embodiments, the 3′ UTR comprises a nucleotide sequence set forth in SEQ ID NO:77, SEQ ID NO:78 or SEQ ID NO:121). In some embodiments, the 5′ UTR comprises a nucleotide sequence set forth in SEQ ID NO:75, SEQ ID NO:76, SEQ ID NO:116, SEQ ID NO: 117, SEQ ID NO: 118, SEQ ID NO: 132 or SEQ ID NO:134 and the 3′ UTR comprises nucleotide sequence set forth in SEQ ID NO:77, SEQ ID NO:78 or SEQ ID NO:121.

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE.

In one aspect, a TEE is a conserved element in a UTR that can promote translational activity of a nucleic acid such as, but not limited to, cap-dependent or cap-independent translation.

MicroRNA (miRNA) Binding Sites

mRNAs of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, mRNAs including such regulatory elements are referred to as including “sensor sequences.” Non-limiting examples of sensor sequences are described in U.S. Publication 2014/0200261, the contents of which are incorporated herein by reference in their entirety.

In some embodiments, an mRNA of the disclosure comprises an open reading frame (ORF) encoding a polypeptide of interest and further comprises one or more miRNA binding site(s). Inclusion or incorporation of miRNA binding site(s) provides for regulation of polynucleotides of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to an mRNA and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, an mRNA of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within an mRNA including in the 5′UTR and/or 3′UTR, that has sufficient complementarity to all or a region of a miRNA to interact with, associate with or bind to the miRNA. In some embodiments, an mRNA of the disclosure comprising an ORF encoding a polypeptide of interest and further comprises one or more miRNA binding site(s). In exemplary embodiments, a 5′UTR and/or 3′UTR of the mRNA comprises the one or more miRNA binding site(s).

A miRNA binding site having sufficient complementarity to a miRNA refers to a degree of complementarity sufficient to facilitate miRNA-mediated regulation of an mRNA, e.g., miRNA-mediated translational repression or degradation of the mRNA. In exemplary aspects of the disclosure, a miRNA binding site having sufficient complementarity to the miRNA refers to a degree of complementarity sufficient to facilitate miRNA-mediated degradation of the mRNA, e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with a miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into an mRNA of the disclosure, the mRNA can be targeted for degradation or reduced translation, provided the miRNA in question is available. This can reduce off-target effects upon delivery of the mRNA. For example, if an mRNA of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the mRNA.

Conversely, miRNA binding sites can be removed from mRNA sequences in which they naturally occur in order to increase protein expression in specific tissues. For example, a binding site for a specific miRNA can be removed from an mRNA to improve protein expression in tissues or cells containing the miRNA.

In one embodiment, an mRNA of the disclosure can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a polynucleotide of the disclosure can include two, three, four, five, six, seven, eight, nine, ten, or more miRNA-binding sites in the 5′-UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc Immune cell specific miRNAs are involved in immunogenicity, autoimmunity, the immune response to infection, inflammation, as well as unwanted immune response after gene therapy and tissue/organ transplantation Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of an mRNA of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the mRNA. The mRNA is then stably expressed in target tissues or cells without triggering cytotoxic elimination.

In one embodiment, binding sites for miRNAs that are known to be expressed in immune cells, in particular, antigen presenting cells, can be engineered into an mRNA of the disclosure to suppress the expression of the polynucleotide in antigen presenting cells through miRNA mediated RNA degradation, subduing the antigen-mediated immune response. Expression of the mRNA is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of an mRNA of the disclosure.

To further drive the selective degradation and suppression in APCs and macrophage, an mRNA of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7α-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p-miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

In some embodiments, an mRNA of the disclosure comprises a miRNA binding site, wherein the miRNA binding site comprises one or more nucleotide sequences selected from 72-74 and 82-83, including one or more copies of any one or more of the miRNA binding site sequences. In some embodiments, an mRNA of the disclosure further comprises at least one, two, three, four, five, six, seven, eight, nine, ten, or more of the same or different miRNA binding sites selected from SEQ ID NOs: 72-74 and 82-83, including any combination thereof.

Some embodiments, an mRNA of the disclosure comprises at least one miR-122 binding site, at least two miR-122 binding sites, at least three miR-122 binding sites, at least four miR-122 binding sites, or at least five miR-122 binding sites. In one aspect, the miRNA binding site binds miR-122 or is complementary to miR-122. In another aspect, the miRNA binding site binds to miR-122-3p or miR-122-5p. In a particular aspect, the miRNA binding site comprises a nucleotide sequence at least 80%, at least 85%, at least 90%, at least 95%, or 100% identical to SEQ ID NO: 74, wherein the miRNA binding site binds to miR-122. In another particular aspect, the miRNA binding site comprises a nucleotide sequence at least 80%, at least 85%, at least 90%, at least 95%, or 100% identical to SEQ ID NO: 83, wherein the miRNA binding site binds to miR-122.

In some embodiments, a miRNA binding site is inserted in the mRNA of the disclosure in any position of the polynucleotide (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the mRNA can be anywhere in the mRNA as long as the insertion of the miRNA binding site in the mRNA does not interfere with the translation of a functional polypeptide in the absence of the corresponding miRNA; and in the presence of the miRNA, the insertion of the miRNA binding site in the mRNA and the binding of the miRNA binding site to the corresponding miRNA are capable of degrading the mRNA or preventing the translation of the mRNA.

In some embodiments, a miRNA binding site is inserted in at least about 30 nucleotides downstream from the stop codon of an ORF in an mRNA of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in an mRNA of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in an mRNA of the disclosure.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The mRNAs of the disclosure can further include this structured 5′UTR in order to enhance microRNA mediated gene regulation.

At least one miRNA binding site can be engineered into the 3′UTR of an mRNA of the disclosure. In this context, at least two, at least three, at least four, at least five, at least six, at least seven, at least eight, at least nine, at least ten, or more miRNA binding sites can be engineered into a 3′UTR of an mRNA of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of an mRNA of the disclosure. In one embodiment, miRNA binding sites incorporated into an mRNA of the disclosure can be the same or can be different miRNA sites. A combination of different miRNA binding sites incorporated into an mRNA of the disclosure can include combinations in which more than one copy of any of the different miRNA sites are incorporated. In another embodiment, miRNA binding sites incorporated into an mRNA of the disclosure can target the same or different tissues in the body. As a non-limiting example, through the introduction of tissue-, cell-type-, or disease-specific miRNA binding sites in the 3′-UTR of an mRNA of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in an mRNA of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

An mRNA of the disclosure can be engineered for more targeted expression in specific tissues, cell types, or biological conditions based on the expression patterns of miRNAs in the different tissues, cell types, or biological conditions. Through introduction of tissue-specific miRNA binding sites, an mRNA of the disclosure can be designed for optimal protein expression in a tissue or cell, or in the context of a biological condition.

In some embodiments, an mRNA of the disclosure can include at least one miRNA in order to dampen expression of the encoded polypeptide in a tissue or cell of interest. As a non-limiting example, an mRNA of the disclosure can include at least one miR-122 binding site in order to dampen expression of an encoded polypeptide of interest in the liver. As another non-limiting example an mRNA of the disclosure can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, an mRNA of the disclosure can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make an mRNA of the disclosure more unstable in antigen presenting cells. Non-limiting examples of these miRNAs include mir-142-5p, mir-142-3p, mir-146a-5p, and mir-146-3p.

In one embodiment, an mRNA of the disclosure comprises at least one miRNA sequence in a region of the mRNA that can interact with an RNA binding protein.

In some embodiments, the mRNA of the disclosure (e.g., a RNA, e.g., an mRNA) comprising (i) a sequence-optimized nucleotide sequence (e.g., an ORF) and (ii) a miRNA binding site (e.g., a miRNA binding site that binds to miR-142).

In some embodiments, the mRNA of the disclosure comprises a uracil-modified sequence encoding a polypeptide disclosed herein and a miRNA binding site disclosed herein, e.g., a miRNA binding site that binds to miR-142. In some embodiments, the uracil-modified sequence encoding a polypeptide comprises at least one chemically modified nucleobase, e.g., 5-methoxyuracil. In some embodiments, at least 95% of a type of nucleobase (e.g., uracil) in a uracil-modified sequence encoding a polypeptide of the disclosure are modified nucleobases. In some embodiments, at least 95% of uracil in a uracil-modified sequence encoding a polypeptide is 5-methoxyuridine. In some embodiments, the mRNA comprising a nucleotide sequence encoding a polypeptide disclosed herein and a miRNA binding site is formulated with a delivery agent, e.g., a compound having the Formula (I), e.g., Compound X.

### Lipid Nanoparticles

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In certain embodiments, the present application provides pharmaceutical compositions comprising:

(a) an mRNA comprising a nucleotide sequence encoding a polypeptide; and

(b) a delivery agent.

In certain embodiments, the present application provides pharmaceutical compositions comprising:

(i) an mRNA encoding a human OX40L polypeptide and an mRNA encoding a human IL-12 polypeptide operably linked to a membrane domain comprising a transmembrane domain;

(ii) an mRNA encoding a human OX40L polypeptide, an mRNA encoding a human IL-15 polypeptide and an mRNA encoding a human IL-15Rα polypeptide;

(iii) an mRNA encoding a human OX40L polypeptide and an mRNA encoding a human IL-15 polypeptide operably linked to a human IL-15Rα polypeptide;

(iv) an mRNA encoding a human IL-12 polypeptide operably linked to a membrane domain comprising a transmembrane domain, an mRNA encoding a human IL-15 polypeptide and an mRNA encoding a human IL-15Rα polypeptide;

(v) an mRNA encoding a human IL-12 polypeptide operably linked to a membrane domain comprising a transmembrane domain and an mRNA encoding a human IL-15 polypeptide operably linked to a human IL-15Rα polypeptide;

(vi) an mRNA encoding a human OX40L polypeptide, an mRNA encoding a human IL-12 polypeptide, an mRNA encoding a human IL-15 polypeptide and an mRNA encoding a human IL-15Rα polypeptide; or

(vii) an mRNA encoding a human OX40L polypeptide, an mRNA encoding a human IL-12 polypeptide operably linked to a membrane domain comprising a transmembrane domain and an mRNA encoding a human IL-15 polypeptide operably linked to a human IL-15Rα polypeptide, and a delivery agent.

In some embodiments, the present application provides pharmaceutical compositions comprising:

(i) a first delivery agent and an mRNA encoding a human OX40L polypeptide;

(ii) a second delivery agent and an mRNA a human IL-15 polypeptide;

(iii) a third delivery agent and an mRNA encoding a human IL-15Rα polypeptide; and

(iv) a fourth delivery agent and an mRNA encoding a human IL-12 polypeptide operably linked to a membrane domain comprising a transmembrane domain.

In some embodiments, the present application provides pharmaceutical compositions comprising:

(i) a first delivery agent and an mRNA encoding a human OX40L polypeptide;

(ii) a second delivery agent and an mRNA encoding a human IL-15 polypeptide operably linked to a human IL-15Rα polypeptide; and

(iii) a third delivery agent and an mRNA encoding a human IL-12 polypeptide operably linked to a membrane domain comprising a transmembrane domain.

Lipid Content of LNPs

In some embodiments, LNPs comprise an (i) ionizable lipid; (ii) sterol or other structural lipid; (iii) a non-cationic helper lipid or phospholipid; a (iv) PEG lipid. These categories of lipids are set forth in more detail below.

(i) Ionizable Lipids

The lipid nanoparticles of the present disclosure include one or more ionizable lipids. In certain embodiments, the ionizable lipids of the disclosure comprise a central amine moiety and at least one biodegradable group. The ionizable lipids described herein may be advantageously used in lipid nanoparticles of the disclosure for the delivery of nucleic acid molecules to mammalian cells or organs. The structures of ionizable lipids set forth below include the prefix I to distinguish them from other lipids of the invention.

In a first aspect of the invention, the compounds described herein are of Formula (I I):

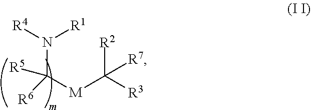

or their N-oxides, or salts or isomers thereof, wherein:

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O) N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected
from —C(O)O—, —OC(O)—, —OC(O)-M″-C(O)O—, —C(O)N(R′)—,
   —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
      R⁷ is selected from the group consisting of C₁₋₃ alkyl, C₂₋₃ alkenyl, and H;
      R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl;

each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13; and wherein when R⁴ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

Another aspect the disclosure relates to compounds of Formula (III):

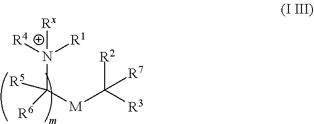

### or its N-oxide,

or a salt or isomer thereof, wherein

or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)₂C(R¹⁰ 2(CH₂)ₙ₋ₒQ,
—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

R^{x} is selected from the group consisting of C₁₋₆ alkyl, C₂₋₆ alkenyl, —(CH₂)ᵥOH, and —(CH₂)ᵥN(R)₂,

wherein v is selected from 1, 2, 3, 4, 5, and 6;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C1-13 alkyl or C₂₋₁₃ alkenyl;
R⁷ is selected from the group consisting of C₁₋₃ alkyl, C₂₋₃ alkenyl, and H;
R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl;

each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In certain embodiments, a subset of compounds of Formula (I) includes those of Formula (IA):

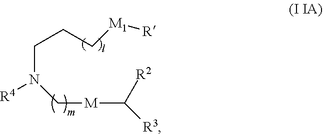

or its N-oxide, or a salt or isomer thereof, wherein 1 is selected from 1, 2, 3, 4, and 5; m is selected from 5, 6, 7, 8, and 9; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR,
heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group, and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

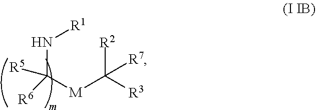

or its N-oxide, or a salt or isomer thereof in which all variables are as defined herein. For example, m is selected from 5, 6, 7, 8, and 9; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. In certain embodiments, a subset of compounds of Formula (I) includes those of Formula (II):

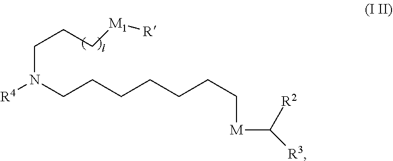

or its N-oxide, or a salt or isomer thereof, wherein 1 is selected from 1, 2, 3, 4, and 5; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR,
heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl.

Another aspect of the disclosure relates to compounds of Formula (I VI):

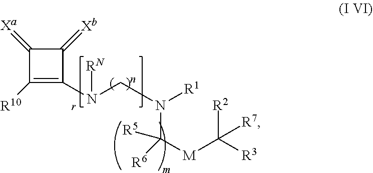

### or its N-oxide,

or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H; each R is independently selected from the group consisting of H, C₁₋₃ alkyl, and C₂₋₃ alkenyl;

RN is H, or C₁₋₃ alkyl;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I;
X^{a} and X^{b} are each independently O or S;
   R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂₀R, —NO₂, —S(O)₂N(R)₂, —N(R)S(O)₂R, —NH(CH₂)ₙ N(R)₂, —NH(CH₂)ₚ₁O(CH₂)_{q1}N(R)₂, —NH(CH₂)ₛ₁R, —N((CH₂)ₛ₁OR)₂, a carbocycle, a heterocycle, aryl and heteroaryl;
   m is selected from 5, 6, 7, 8, 9, 10, 11, 12, and 13;
   n is selected from 1, 2, 3, 4, 5, 6, 7, 8, 9, and 10;
   r is 0 or 1;
   t¹ is selected from 1, 2, 3, 4, and 5;
   p¹ is selected from 1, 2, 3, 4, and 5;
   q¹ is selected from 1, 2, 3, 4, and 5; and
   s¹ is selected from 1, 2, 3, 4, and 5.

In one embodiment, a subset of compounds of Formula (VI) includes those of Formula (VI-a):

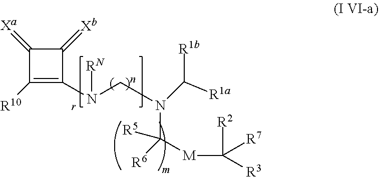

### or its N-oxide,

or a salt or isomer thereof, wherein

R^{1a} and R^{1b} are independently selected from the group consisting of C₁₋₁₄ alkyl and C₂₋₁₄ alkenyl; and

R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle.

In another embodiment, a subset of compounds of Formula (VI) includes those of Formula (VII):

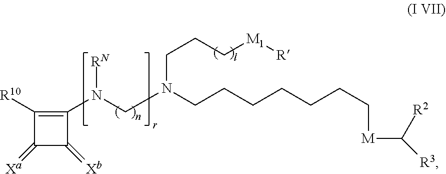

or its N-oxide, or a salt or isomer thereof, wherein

1 is selected from 1, 2, 3, 4, and 5;

M₁ is a bond or M′; and

R² and R³ are independently selected from the group consisting of H, C₁₋₁₄ alkyl, and C₂₋₁₄ alkenyl.

In another embodiment, a subset of compounds of Formula (I VI) includes those of Formula (I VIII):

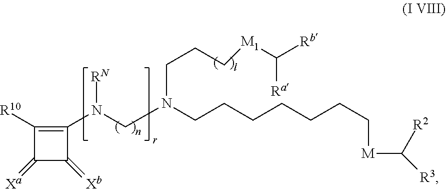

or its N-oxide, or a salt or isomer thereof, wherein

1 is selected from 1, 2, 3, 4, and 5;

M₁ is a bond or M′; and

R^{a′} and R^{b′} are independently selected from the group consisting of C₁₋₁₄ alkyl and C₂-14 alkenyl; and

R² and R³ are independently selected from the group consisting of C₁₋₁₄ alkyl, and C₂-14 alkenyl.

The compounds of any one of formula (I I), (I IA), (I VI), (I VI-a), (I VII) or (I VIII) include one or more of the following features when applicable.

In some embodiments, M₁ is M′.

In some embodiments, M and M′ are independently —C(O)O— or —OC(O)—.

In some embodiments, at least one of M and M′ is —C(O)O— or —OC(O)—.

In certain embodiments, at least one of M and M′ is —OC(O)—.

In certain embodiments, M is —OC(O)— and M′ is —C(O)O—. In some embodiments, M is —C(O)O— and M′ is —OC(O)—. In certain embodiments, M and M′ are each —OC(O)—. In some embodiments, M and M′ are each —C(O)O—.

In certain embodiments, at least one of M and M′ is —OC(O)-M″-C(O)O—.

In some embodiments, M and M′ are independently —S—S—.

In some embodiments, at least one of M and M′ is —S—S.

In some embodiments, one of M and M′ is —C(O)O— or —OC(O)— and the other is —S—S—. For example, M is —C(O)O— or —OC(O)— and M′ is —S—S— or M′ is —C(O)O—, or —OC(O)— and M is —S—S—.

In some embodiments, one of M and M′ is —OC(O)-M″-C(O)O—, in which M″ is a bond, C₁₋₁₃ alkyl or C₂₋₁₃ alkenyl. In other embodiments, M″ is C₁₋₆ alkyl or C₂₋₆ alkenyl. In certain embodiments, M″ is C₁₋₄ alkyl or C₂₋₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, 1 is 1, 3, or 5.

In some embodiments, R⁴ is hydrogen.

In some embodiments, R⁴ is not hydrogen.

In some embodiments, R⁴ is unsubstituted methyl or —(CH₂)ₙQ, in which Q is OH, —NHC(S)N(R)₂, —NHC(O)N(R)₂, —N(R)C(O)R, or —N(R)S(O)₂R.

In some embodiments, Q is OH.

In some embodiments, Q is —NHC(S)N(R)₂.

In some embodiments, Q is —NHC(O)N(R)₂.

In some embodiments, Q is —N(R)C(O)R.

In some embodiments, Q is —N(R)S(O)₂R.

In some embodiments, Q is —O(CH₂)ₙN(R)₂.

In some embodiments, Q is —O(CH₂)ₙOR.

In some embodiments, Q is —N(R)R⁸.

In some embodiments, Q is —NHC(═NR⁹)N(R)₂.

In some embodiments, Q is —NHC(═CHR⁹)N(R)₂.

In some embodiments, Q is —OC(O)N(R)₂.

In some embodiments, Q is —N(R)C(O)OR.

In some embodiments, n is 2.

In some embodiments, n is 3.

In some embodiments, n is 4.

In some embodiments, M₁ is absent.

In some embodiments, at least one R⁵ is hydroxyl. For example, one R⁵ is hydroxyl.

In some embodiments, at least one R⁶ is hydroxyl. For example, one R⁶ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen.

In some embodiments, RX is C₁₋₆ alkyl. In some embodiments, RX is C₁₋₃ alkyl. For example, RX is methyl. For example, RX is ethyl. For example, RX is propyl.

In some embodiments, RX is —(CH₂)ᵥOH and, v is 1, 2 or 3. For example, RX is methanoyl. For example, RX is ethanoyl. For example, RX is propanoyl.

In some embodiments, RX is —(CH₂)ᵥN(R)₂, v is 1, 2 or 3 and each R is H or methyl. For example, R^{x} is methanamino, methylmethanamino, or dimethylmethanamino. For example, R^{x} is aminomethanyl, methylaminomethanyl, or dimethylaminomethanyl. For example, R^{x} is aminoethanyl, methylaminoethanyl, or dimethylaminoethanyl. For example, R^{x} is aminopropanyl, methylaminopropanyl, or dimethylaminopropanyl.

In some embodiments, R′ is C₁₋₁₈ alkyl, C₂₋₁₈ alkenyl, —R*YR″, or -YR″.

In some embodiments, R² and R³ are independently C₃₋₁₄ alkyl or C₃₋₁₄ alkenyl. In some embodiments, R^{1b} is C₁₋₁₄ alkyl. In some embodiments, R^{1b} is C₂₋₁₄ alkyl. In some embodiments, R^{1b} is C₃₋₁₄ alkyl. In some embodiments, R^{1b} is C₁₋₈ alkyl. In some embodiments, R^{1b} is C₁₋₅ alkyl. In some embodiments, R^{1b} is C₁₋₃ alkyl. In some embodiments, R^{1b} is selected from C₁ alkyl, C₂ alkyl, C₃ alkyl, C₄ alkyl, and C₅ alkyl. For example, in some embodiments, R^{1b} is C₁ alkyl. For example, in some embodiments, R^{1b} is C₂ alkyl. For example, in some embodiments, R^{1b} is C₃ alkyl. For example, in some embodiments, R^{1b} is C₄ alkyl. For example, in some embodiments, R^{1b} is C₅ alkyl.

In some embodiments, R¹ is different from —(CHR⁵R⁶)ₘ-M-CR²R³R⁷.

In some embodiments, —CHR^{1a}R^{1b}— is different from —(CHR⁵R⁶)ₘ-M-CR²R³R⁷.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl.

In some embodiments, R^{b′} is C₁₋₁₄ alkyl. In some embodiments, R^{b′} is C₂₋₁₄ alkyl. In some embodiments, R^{b′} is C₃₋₁₄ alkyl. In some embodiments, R^{b′} is C₁₋₈ alkyl. In some embodiments, R^{b′} is C₁s alkyl. In some embodiments, R^{b′} is C₁₋₃ alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

In one embodiment, the compounds of Formula (I) are of Formula (IIa):

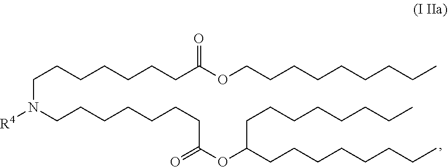

or their N-oxides, or salts or isomers thereof, wherein R⁴ is as described herein.

In another embodiment, the compounds of Formula (I) are of Formula (IIb):

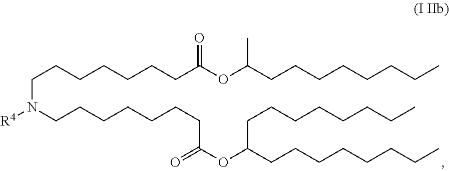

or their N-oxides, or salts or isomers thereof, wherein R⁴ is as described herein.

In another embodiment, the compounds of Formula (I) are of Formula (IIc) or (IIe):

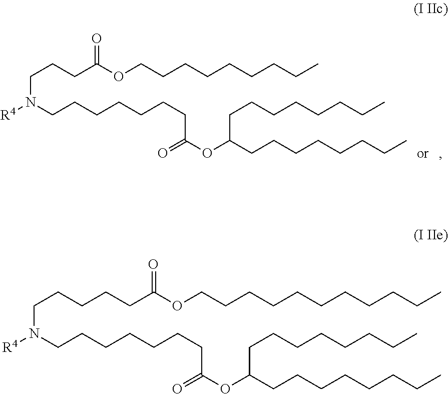

or their N-oxides, or salts or isomers thereof, wherein R⁴ is as described herein.

In another embodiment, the compounds of Formula (I I) are of Formula (I IIf):

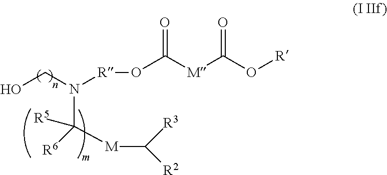

or their N-oxides, or salts or isomers thereof,

wherein M is —C(O)O— or —OC(O)—, M″ is C₁₋₆ alkyl or C₂₋₆ alkenyl, R² and R³ are independently selected from the group consisting of C₅₋₁₄ alkyl and C₅₋₁₄ alkenyl, and n is selected from 2, 3, and 4.

In a further embodiment, the compounds of Formula (I I) are of Formula (IId):

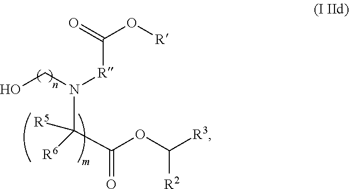

or their N-oxides, or salts or isomers thereof, wherein n is 2, 3, or 4; and m, R′, R″, and R² through R₆ are as described herein. For example, each of R² and R³ may be independently selected from the group consisting of C₅₋₁₄ alkyl and C₅₋₁₄ alkenyl.

In a further embodiment, the compounds of Formula (I) are of Formula (IIg):

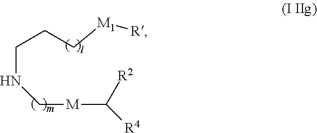

or their N-oxides, or salts or isomers thereof, wherein 1 is selected from 1, 2, 3, 4, and 5; m is selected from 5, 6, 7, 8, and 9; M₁ is a bond or M′; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R² and R³ are independently selected from the group consisting of C₅₋₁₄ alkyl and C₅₋₁₄ alkenyl.

In another embodiment, a subset of compounds of Formula (I VI) includes those of Formula (I VIIa):

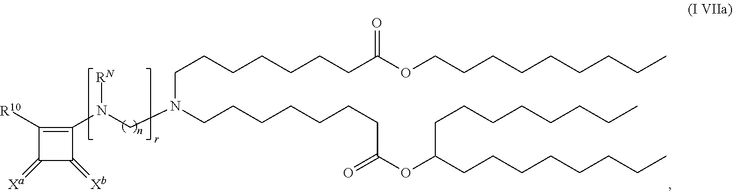

or its N-oxide, or a salt or isomer thereof.

In another embodiment, a subset of compounds of Formula (I VI) includes those of Formula (I VIIIa):

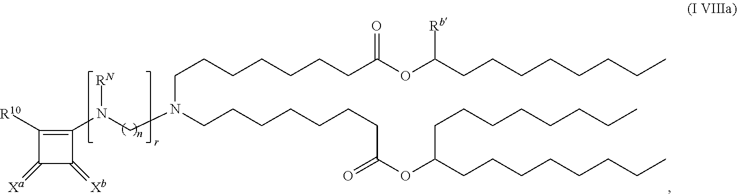

or its N-oxide, or a salt or isomer thereof.

In another embodiment, a subset of compounds of Formula (I VI) includes those of Formula (I VIIIb):

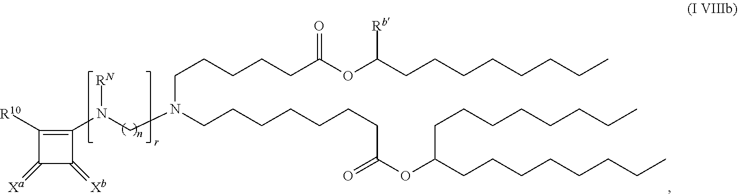

or its N-oxide, or a salt or isomer thereof.

In another embodiment, a subset of compounds of Formula (I VI) includes those of Formula (I VIIb-1):

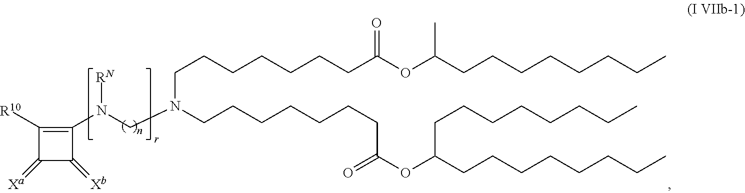

or its N-oxide, or a salt or isomer thereof.

In another embodiment, a subset of compounds of Formula (I VI) includes those of Formula (I VIIb-2):

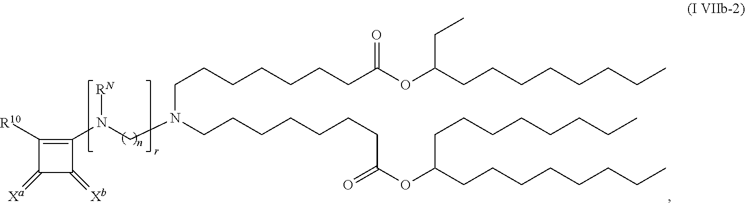

or its N-oxide, or a salt or isomer thereof.

In another embodiment, a subset of compounds of Formula (I VI) includes those of Formula (I VIIb-3):

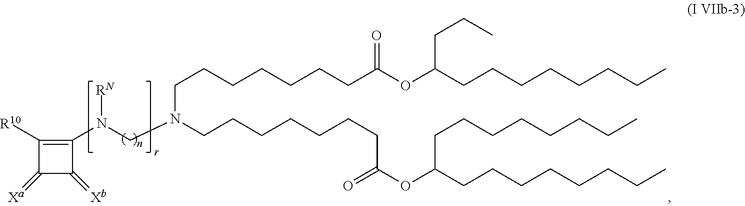

or its N-oxide, or a salt or isomer thereof. In another embodiment, a subset of compounds of Formula (VI) includes those of Formula (VIIc):

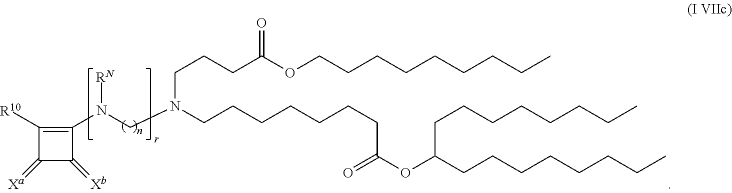

In another embodiment, a subset of compounds of Formula (I VI) includes those of Formula (VIId):

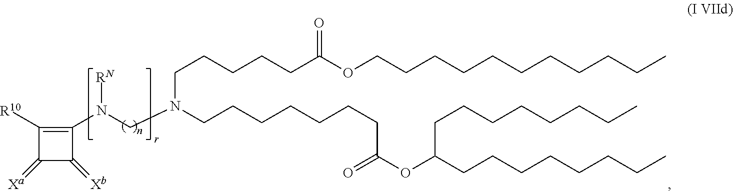

or its N-oxide, or a salt or isomer thereof.

In another embodiment, a subset of compounds of Formula (I VI) includes those of Formula (I VIIIc):

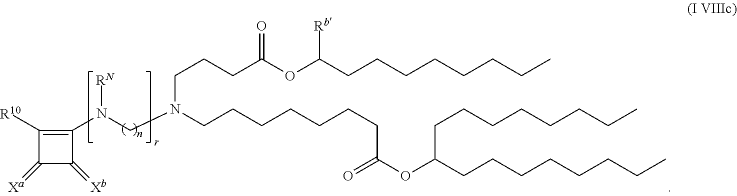

In another embodiment, a subset of compounds of Formula I VI) includes those of Formula (I VIIId):

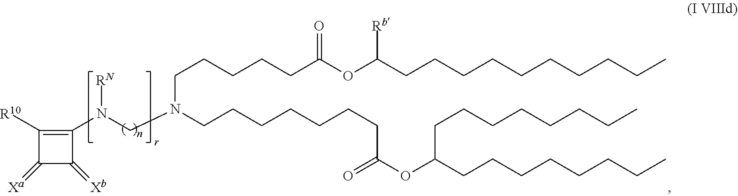

or its N-oxide, or a salt or isomer thereof.

The compounds of any one of formulae (I I), (I IA), (I IB), (I II), (I Ha), (I IIb), (I IIc), (I IId), (IIe), (I IIf), (I IIg), I (III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), or (I VIIId) include one or more of the following features when applicable.

In some embodiments, R⁴ is selected from the group consisting of a C₃₋₆ carbocycle, —(CH₂)ₙQ, —(CH₂)ₙCHQR,)—(CH₂)ₒC(R¹⁰ 2(CH₂)ₙ₋ₒQ, —CHQR, and —CQ(R)₂, where Q is selected from a C₃₋₆ carbocycle, 5- to 14-membered aromatic or non-aromatic heterocycle having one or more heteroatoms selected from N, O, S, and P, —OR, —O(CH₂)ₙN(R)₂, —C(O)OR,

—OC(O)R, —CX₃, —CX₂H, —CXH₂, —CN, —N(R)₂, —N(R)S(O)₂R⁸, —C(O)N(R)₂, —N(R)C(O)R, —N(R)S(O)₂R, —N(R)C(O)N(R)₂, —N(R)C(S)N(R)₂, and —C(R)N(R)₂C(O)OR, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR,)—(CH₂)ₒC(R¹⁰ 2(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃,

—CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR,)—(CH₂)ₒC(R¹⁰ 2(CH₂)ₙ₋ₒQ, —CHQR, and —CQ(R)₂, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5; and when Q is a 5- to 14-membered heterocycle and (i) R⁴ is —(CH₂)ₙQ in which n is 1 or 2, or (ii) R⁴ is —(CH₂)ₙCHQR in which n is 1, or (iii) R⁴ is —CHQR, and —CQ(R)₂, then Q is either a 5- to 14-membered heteroaryl or 8- to 14-membered heterocycloalkyl.

In another embodiment, R⁴ is selected from the group consisting of a C₃₋₆ carbocycle, —(CH₂)ₙQ, —(CH₂)ₙCHQR,)—(CH₂)ₒC(R¹⁰ 2(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃,

—CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5.

In another embodiment, R⁴ is —(CH₂)ₙQ, where Q is —N(R)S(O)₂R⁸ and n is selected from 1, 2, 3, 4, and 5. In a further embodiment, R⁴ is —(CH₂)ₙ₋ₒQ, where Q is —N(R)S(O)₂R⁸, in which R⁸ is a C₃₋₆ carbocycle such as C₃₋₆ cycloalkyl, and n is selected from 1, 2, 3, 4, and 5. For example, R⁴ is —(CH₂)₃NHS(O)₂R⁸ and R⁸ is cyclopropyl.

In another embodiment, R⁴ is —(CH₂)ₒC(R¹⁰)₂(CH₂)ₙ₋ₒQ, where Q is —N(R)C(O)R, n is selected from 1, 2, 3, 4, and 5, and o is selected from 1, 2, 3, and 4. In a further embodiment, R⁴ is —(CH₂)ₒC(R¹⁰ 2(CH₂)ₙ₋ₒQ, where Q is —N(R)C(O)R, wherein R is C₁-C₃ alkyl and n is selected from 1, 2, 3, 4, and 5, and o is selected from 1, 2, 3, and 4. In a another embodiment, R⁴ is —(CH₂)ₒC(R¹⁰ 2(CH₂)ₙ₋ₒQ, where Q is —N(R)C(O)R, wherein R is C₁-C₃ alkyl, n is 3, and o is 1. In some embodiments, R¹⁰ is H, OH, C₁₋₃ alkyl, or C₂₋₃ alkenyl. For example, R⁴ is 3-acetamido-2,2-dimethylpropyl.

In some embodiments, one R¹⁰ is H and one R¹⁰ is C₁₋₃ alkyl or C₂₋₃ alkenyl. In another embodiment, each R¹⁰ is C₁₋₃ alkyl or C₂₋₃ alkenyl. In another embodiment, each R¹⁰ is C₁₋₃ alkyl (e.g. methyl, ethyl or propyl). For example, one R¹⁰ is methyl and one R¹⁰ is ethyl or propyl. For example, one R¹⁰ is ethyl and one R¹⁰ is methyl or propyl. For example, one R¹⁰ is propyl and one R¹⁰ is methyl or ethyl. For example, each R¹⁰ is methyl. For example, each R¹⁰ is ethyl. For example, each R¹⁰ is propyl.

In some embodiments, one R¹⁰ is H and one R¹⁰ is OH. In another embodiment, each R¹⁰ is OH.

In another embodiment, R⁴ is unsubstituted C₁₋₄ alkyl, e.g., unsubstituted methyl. In another embodiment, R⁴ is hydrogen.

In certain embodiments, the disclosure provides a compound having the Formula (I), wherein R⁴ is —(CH₂)ₙQ or —(CH₂)ₙCHQR, where Q is —N(R)₂, and n is selected from 3, 4, and 5.

In certain embodiments, the disclosure provides a compound having the Formula (I), wherein R⁴ is selected from the group consisting of —(CH₂)ₙQ, —(CH₂)ₙCHQR, —CHQR, and —CQ(R)₂, where Q is —N(R)₂, and n is selected from 1, 2, 3, 4, and 5.

In certain embodiments, the disclosure provides a compound having the Formula (I), wherein R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle, and R⁴ is —(CH₂)ₙQ or —(CH₂)ₙCHQR, where Q is —N(R)₂, and n is selected from 3, 4, and 5.

In certain embodiments, R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle. In some embodiments, R² and R³ are independently selected from the group consisting of C₂₋₁₄ alkyl, and C₂₋₁₄ alkenyl. In some embodiments, R² and R³ are independently selected from the group consisting of —R*YR″, —YR″, and —R*OR″. In some embodiments, R² and R³ together with the atom to which they are attached, form a heterocycle or carbocycle.

In some embodiments, R¹ is selected from the group consisting of C₅₋₂₀ alkyl and C₅₋₂₀ alkenyl. In some embodiments, R′ is C₅₋₂₀ alkyl substituted with hydroxyl.

In other embodiments, R′ is selected from the group consisting of —R*YR″, —YR″, and —R″M′R′.

In certain embodiments, R′ is selected from -R*YR″ and -YR″. In some embodiments, Y is a cyclopropyl group. In some embodiments, R* is C₈ alkyl or C₈ alkenyl. In certain embodiments, R″ is C₃₋₁₂ alkyl. For example, R″ may be C₃ alkyl. For example, R″ may be C₄₋₈ alkyl (e.g., C₄, C₅, C₆, C₇, or C₈ alkyl).

In some embodiments, R is (CH₂)_{q}OR*, q is selected from 1, 2, and 3, and R* is C₁₋₁₂ alkyl substituted with one or more substituents selected from the group consisting of amino, C₁-C₆ alkylamino, and C₁-C₆ dialkylamino. For example, R is (CH₂)_{q}OR*, q is selected from 1, 2, and 3 and R* is C₁₋₁₂ alkyl substituted with C₁-C₆dialkylamino. For example, R is (CH₂)_{q}OR*, q is selected from 1, 2, and 3 and R* is C₁₋₃ alkyl substituted with C₁-C₆ dialkylamino. For example, R is (CH₂)_{q}OR*, q is selected from 1, 2, and 3 and R* is C₁₋₃ alkyl substituted with dimethylamino (e.g., dimethylaminoethanyl).

In some embodiments, R¹ is C₅₋₂₀ alkyl. In some embodiments, R¹ is C₆ alkyl. In some embodiments, R¹ is C₈ alkyl. In other embodiments, R¹ is C₉ alkyl. In certain embodiments, R¹ is C₁₄ alkyl. In other embodiments, R¹ is C₁₈ alkyl.

In some embodiments, R¹ is C₂₁₋₃₀ alkyl. In some embodiments, R¹ is C₂₆ alkyl. In some embodiments, R¹ is C₂₈ alkyl. In certain embodiments, R¹ is

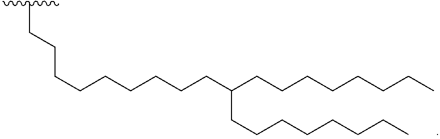 [SMILES: *CCCCCCCCCC(CCCCCCCC)CCCCCCCC]

In some embodiments, R¹ is C₅₋₂₀ alkenyl. In certain embodiments, R¹ is C₁₈ alkenyl. In some embodiments, R¹ is linoleyl.

In certain embodiments, R¹ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl, or heptadeca-9-yl). In certain embodiments, R¹ is

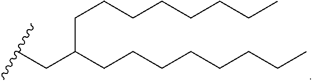 [SMILES: *CC(CCCCCCCC)CCCCCCCC]

In certain embodiments, R¹ is unsubstituted C₅₋₂₀ alkyl or C₅₋₂₀ alkenyl. In certain embodiments, R′ is substituted C₅₋₂₀ alkyl or C₅₋₂₀ alkenyl (e.g., substituted with a C₃₋₆ carbocycle such as 1-cyclopropylnonyl or substituted with OH or alkoxy). For example, R¹ is

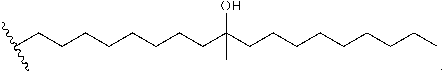 [SMILES: *CCCCCCCCC(C)(O)CCCCCCCCC]

In other embodiments, R¹ is —R″M′R′. In certain embodiments, M′ is —OC(O)-M″-C(O)O—. For example, R¹ is

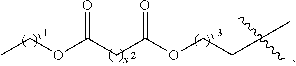

wherein x¹ is an integer between 1 and 13 (e.g., selected from 3, 4, 5, and 6), x² is an integer between 1 and 13 (e.g., selected from 1, 2, and 3), and x³ is an integer between 2 and 14 (e.g., selected from 4, 5, and 6). For example, x¹ is selected from 3, 4, 5, and 6, x² is selected from 1, 2, and 3, and x³ is selected from 4, 5, and 6.

In other embodiments, R¹ is different from —(CHR⁵R⁶)ₘ-M-CR²R³R⁷.

In some embodiments, R′ is selected from -R*YR″ and -YR″. In some embodiments, Y is C₃₋₈ cycloalkyl. In some embodiments, Y is C₆₋₁₀ aryl. In some embodiments, Y is a cyclopropyl group. In some embodiments, Y is a cyclohexyl group. In certain embodiments, R* is C₁ alkyl.

In some embodiments, R″ is selected from the group consisting of C₃₋₁₂ alkyl and C₃₋₁₂ alkenyl. In some embodiments, R″ is C₈ alkyl. In some embodiments, R″ adjacent to Y is C₁ alkyl. In some embodiments, R″ adjacent to Y is C₄₋₉ alkyl (e.g., C₄, C₅, C₆, C₇ or C₈ or C₉ alkyl).

In some embodiments, R″ is substituted C₃₋₁₂ (e.g., C₃₋₁₂ alkyl substituted with, e.g., an hydroxyl). For example, R″ is

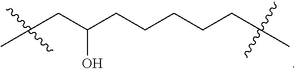 [SMILES: *CCCCCC(O)C*]

In some embodiments, R′ is selected from C₄ alkyl and C₄ alkenyl. In certain embodiments, R′ is selected from C₅ alkyl and C₅ alkenyl. In some embodiments, R′ is selected from C₆ alkyl and C₆ alkenyl. In some embodiments, R′ is selected from C₇ alkyl and C₇ alkenyl. In some embodiments, R′ is selected from C₉ alkyl and C₉ alkenyl.

In some embodiments, R′ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl, each of which is either linear or branched.

In some embodiments, R′ is linear. In some embodiments, R′ is branched.

In some embodiments, R′ is

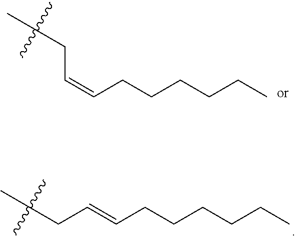

In some embodiments, R′ is

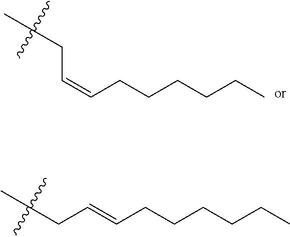

and M′ is —OC(O)—. In other embodiments, R′ is

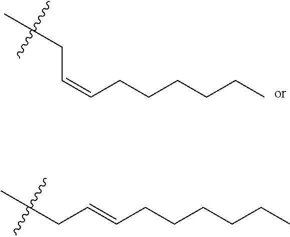

### and M′ is —C(O)O—.

In other embodiments, R′ is selected from C₁₁ alkyl and C₁₁ alkenyl. In other embodiments, R′ is selected from C₁₂ alkyl, C₁₂ alkenyl, C₁₃ alkyl, C₁₃ alkenyl, C₁₄ alkyl, C₁₄ alkenyl, C₁₅ alkyl, C₁₅ alkenyl, C₁₆ alkyl, C₁₆ alkenyl, C₁₇ alkyl, C₁₇ alkenyl, C₁₈ alkyl, and C₁₈ alkenyl. In certain embodiments, R′ is linear C₄₋₁₈ alkyl or C₄₋₁₈ alkenyl. In certain embodiments, R′ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl or heptadeca-9-yl). In certain embodiments, R′ is

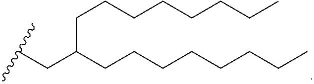 [SMILES: *CC(CCCCCCCC)CCCCCCCC]

In certain embodiments, R′ is unsubstituted C₁₋₁₈ alkyl. In certain embodiments, R′ is substituted C₁₋₁₈ alkyl (e.g., C₁₋₁₅ alkyl substituted with, e.g., an alkoxy such as methoxy, or a C₃₋₆ carbocycle such as 1-cyclopropylnonyl, or C(O)O-alkyl or OC(O)-alkyl such as C(O)OCH3 or OC(O)CH₃). For example, R′ is

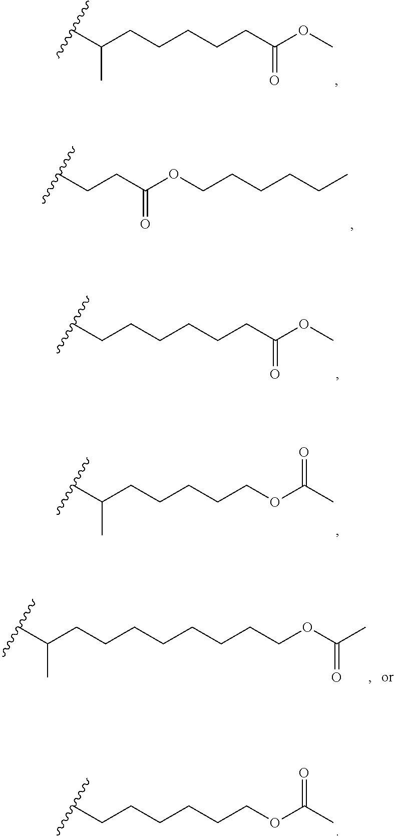 [SMILES: *C(C)CCCCCC(=O)OC ; *CCC(=O)OCCCCCC ; *CCCCCCC(=O)OC ; *C(C)CCCCCOC(C)=O ; *C(C)CCCCCCCCOC(C)=O ; *CCCCCCOC(C)=O]

In certain embodiments, R′ is branched C₁₋₁₈ alkyl. For example, R′ is

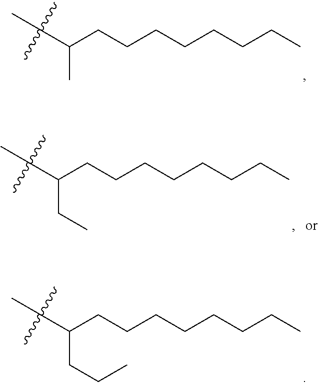 [SMILES: *C(C)CCCCCCCC ; *C(CC)CCCCCCCC ; *C(CCC)CCCCCCCC]

In some embodiments, R″ is selected from the group consisting of C₃₋₁₅ alkyl and C₃₋₁₅ alkenyl. In some embodiments, R″ is C₃ alkyl, C₄ alkyl, C₅ alkyl, C₆ alkyl, C₇ alkyl, or C₈ alkyl. In some embodiments, R″ is C₉ alkyl, C₁₀ alkyl, C₁₁ alkyl, C₁₂ alkyl, C₁₃ alkyl, C₁₄ alkyl, or C₁₅ alkyl.

In some embodiments, M′ is —C(O)O—. In some embodiments, M′ is —OC(O)—. In some embodiments, M′ is —OC(O)-M″-C(O)O—.

In some embodiments, M′ is —C(O)O—, —OC(O)—, or —OC(O)-M″-C(O)O—. In some embodiments wherein M′ is —OC(O)-M″-C(O)O—, M″ is C₁₋₄ alkyl or C₂₋₄ alkenyl.

In other embodiments, M′ is an aryl group or heteroaryl group. For example, M′ may be selected from the group consisting of phenyl, oxazole, and thiazole.

In some embodiments, M is —C(O)O—. In some embodiments, M is —OC(O)—. In some embodiments, M is —C(O)N(R′)—. In some embodiments, M is —P(O)(OR′)O—. In some embodiments, M is —OC(O)-M″-C(O)O—.

In some embodiments, M is —C(O). In some embodiments, M is —OC(O)— and M′ is —C(O)O—. In some embodiments, M is —C(O)O— and M′ is —OC(O)—. In some embodiments, M and M′ are each —OC(O)—. In some embodiments, M and M′ are each —C(O)O—.

In other embodiments, M is an aryl group or heteroaryl group. For example, M may be selected from the group consisting of phenyl, oxazole, and thiazole.

In some embodiments, M is the same as M′. In other embodiments, M is different from M′.

In some embodiments, M″ is a bond. In some embodiments, M″ is C₁₋₁₃ alkyl or C₂₋₁₃ alkenyl. In some embodiments, M″ is C₁₋₆ alkyl or C₂₋₆ alkenyl. In certain embodiments, M″ is linear alkyl or alkenyl. In certain embodiments, M″ is branched, e.g., —CH(CH₃)CH₂—.

In some embodiments, each R⁵ is H. In some embodiments, each R⁶ is H. In certain such embodiments, each R⁵ and each R⁶ is H.

In some embodiments, R⁷ is H. In other embodiments, R⁷ is C₁₋₃ alkyl (e.g., methyl, ethyl, propyl, or i-propyl).

In some embodiments, R² and R³ are independently C₅₋₁₄ alkyl or C₅₋₁₄ alkenyl.

In some embodiments, R² and R³ are the same. In some embodiments, R² and R³ are C₈ alkyl. In certain embodiments, R² and R³ are C₂ alkyl. In other embodiments, R² and R³ are C₃ alkyl. In some embodiments, R² and R³ are C₄ alkyl. In certain embodiments, R² and R³ are C₅ alkyl. In other embodiments, R² and R³ are C₆ alkyl. In some embodiments, R² and R³ are C₇ alkyl.

In other embodiments, R² and R³ are different. In certain embodiments, R² is C₈ alkyl. In some embodiments, R³ is C₁₋₇ (e.g., C₁, C₂, C₃, C₄, C₅, C₆, or C₇ alkyl) or C₉ alkyl.

In some embodiments, R³ is C₁ alkyl. In some embodiments, R³ is C₂ alkyl. In some embodiments, R³ is C₃ alkyl. In some embodiments, R³ is C₄ alkyl. In some embodiments, R³ is C₅ alkyl. In some embodiments, R³ is C₆ alkyl. In some embodiments, R³ is C₇ alkyl.

In some embodiments, R³ is C₉ alkyl.

In some embodiments, R⁷ and R³ are H.

In certain embodiments, R² is H.

In some embodiments, m is 5, 6, 7, 8, or 9. In some embodiments, m is 5, 7, or 9. For example, in some embodiments, m is 5. For example, in some embodiments, m is 7. For example, in some embodiments, m is 9.

In some embodiments, R⁴ is selected from —(CH₂)ₙQ and —(CH₂)ₙCHQR.

In some embodiments, Q is selected from the group consisting

of —OR, —OH, —O(CH₂)ₙN(R)₂, —OC(O)R, —CX₃, —CN, —N(R)C(O)R, —N(H)C(O)R, —N(R)S(O)₂ R,

—N(H)S(O)₂R, —N(R)C(O)N(R)₂, —N(H)C(O)N(R)₂, —N(H)C(O)N(H)(R), —N(R)C(S)N(R)₂,

—N(H)C(S)N(R)₂, —N(H)C(S)N(H)(R), —C(R)N(R)₂C(O)OR, —N(R)S(O)₂R⁸, a carbocycle, and a heterocycle.

In certain embodiments, Q is —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, or —N(R)C(O)OR.

In certain embodiments, Q is —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, or —N(OR)C(═CHR⁹)N(R)₂.

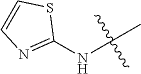 [SMILES: *Nc1nccs1]

In certain embodiments, Q is thiourea or an isostere thereof, e.g., or —NHC(═NR⁹)N(R)₂.

In certain embodiments, Q is —C(═NR⁹)N(R)₂. For example, when Q is —C(═NR⁹)N(R)₂, n is 4 or 5. For example, R⁹ is —S(O)₂N(R)₂.

In certain embodiments, Q is —C(═NR⁹)R or —C(O)N(R)OR, e.g., —CH(═N—OCH₃), —C(O)NH—OH, —C(O)NH—OCH₃, —C(O)N(CH₃)—OH, or —C(O)N(CH₃)—OCH₃.

In certain embodiments, Q is —OH.

In certain embodiments, Q is a substituted or unsubstituted 5- to 10-membered heteroaryl, e.g., Q is a triazole, an imidazole, a pyrimidine, a purine, 2-amino-1,9-dihydro-6H-purin-6-one-9-yl (or guanin-9-yl), adenin-9-yl, cytosin-1-yl, or uracil-1-yl, each of which is optionally substituted with one or more substituents selected from alkyl, OH, alkoxy, -alkyl-OH, -alkyl-O-alkyl, and the substituent can be further substituted. In certain embodiments, Q is a substituted 5- to 14-membered heterocycloalkyl, e.g., substituted with one or more substituents selected from oxo (═O), OH, amino, mono- or di-alkylamino, and C₁₋₃ alkyl. For example, Q is 4-methylpiperazinyl, 4-(4-methoxybenzyl)piperazinyl, isoindolin-2-yl-1,3-dione, pyrrolidin-1-yl-2,5-dione, or imidazolidin-3-yl-2,4-dione.

In certain embodiments, Q is —NHR⁸, in which R⁸ is a C₃₋₆ cycloalkyl optionally substituted with one or more substituents selected from oxo (═O), amino (NH₂), mono- or di-alkylamino, C₁₋₃ alkyl and halo. For example, R⁸ is cyclobutenyl, e.g., 3-(dimethylamino)-cyclobut-3-ene-4-yl-1,2-dione. In further embodiments, R⁸ is a C₃₋₆ cycloalkyl optionally substituted with one or more substituents selected from oxo (═O), thio (═S), amino (NH₂), mono- or di-alkylamino, C₁₋₃ alkyl, heterocycloalkyl, and halo, wherein the mono- or di-alkylamino, C₁₋₃ alkyl, and heterocycloalkyl are further substituted. For example R⁸ is cyclobutenyl substituted with one or more of oxo, amino, and alkylamino, wherein the alkylamino is further substituted, e.g., with one or more of C₁₋₃ alkoxy, amino, mono- or di-alkylamino, and halo. For example, R⁸ is 3-(((dimethylamino)ethyeamino)cyclobut-3-enyl-1,2-dione. For example R⁸ is cyclobutenyl substituted with one or more of oxo, and alkylamino. For example, R⁸ is 3-(ethylamino)cyclobut-3-ene-1,2-dione. For example R⁸ is cyclobutenyl substituted with one or more of oxo, thio, and alkylamino. For example R⁸ is 3-(ethylamino)-4-thioxocyclobut-2-en-1-one or 2-(ethylamino)-4-thioxocyclobut-2-en-1-one. For example R⁸ is cyclobutenyl substituted with one or more of thio, and alkylamino. For example R⁸ is 3-(ethylamino)cyclobut-3-ene-1,2-dithione. For example R⁸ is cyclobutenyl substituted with one or more of oxo and dialkylamino. For example R⁸ is 3-(diethylamino)cyclobut-3-ene-1,2-dione. For example, R⁸ is cyclobutenyl substituted with one or more of oxo, thio, and dialkylamino. For example, R⁸ is 2-(diethylamino)-4-thioxocyclobut-2-en-1-one or 3-(diethylamino)-4-thioxocyclobut-2-en-1-one. For example, R⁸ is cyclobutenyl substituted with one or more of thio, and dialkylamino. For example, R⁸ is 3-(diethylamino)cyclobut-3-ene-1,2-dithione. For example, R⁸ is cyclobutenyl substituted with one or more of oxo and alkylamino or dialkylamino, wherein alkylamino or dialkylamino is further substituted, e.g. with one or more alkoxy. For example, R⁸ is 3-(bis(2-methoxyethyl)amino)cyclobut-3-ene-1,2-dione. For example, R⁸ is cyclobutenyl substituted with one or more of oxo, and heterocycloalkyl. For example, R⁸ is cyclobutenyl substituted with one or more of oxo, and piperidinyl, piperazinyl, or morpholinyl. For example, R⁸ is cyclobutenyl substituted with one or more of oxo, and heterocycloalkyl, wherein heterocycloalkyl is further substituted, e.g., with one or more C₁₋₃ alkyl. For example, R⁸ is cyclobutenyl substituted with one or more of oxo, and heterocycloalkyl, wherein heterocycloalkyl (e.g., piperidinyl, piperazinyl, or morpholinyl) is further substituted with methyl.

In certain embodiments, Q is —NHR⁸, in which R⁸ is a heteroaryl optionally substituted with one or more substituents selected from amino (NH₂), mono- or di-alkylamino, C₁₋₃ alkyl and halo. For example, R⁸ is thiazole or imidazole.

In certain embodiments, Q is —NHC(═NR⁹)N(R)₂ in which R⁹ is CN, C₁₋₆ alkyl, NO₂, —S(O)₂N(R)₂, —OR, —S(O)₂R, or H. For example, Q is —NHC(═NR⁹)N(CH₃)₂, —NHC(═NR⁹)NHCH₃, —NHC(═NR⁹)NH₂. In some embodiments, Q is —NHC(═NR⁹)N(R)₂ in which R⁹ is CN and R is C₁₋₃ alkyl substituted with mono- or di-alkylamino, e.g., R is ((dimethylamino)ethyl)amino. In some embodiments, Q is —NHC(═NR⁹)N(R)₂ in which R⁹ is C₁₋₆ alkyl, NO₂, —S(O)₂N(R)₂, —OR, —S(O)₂R, or H and R is C₁₋₃ alkyl substituted with mono- or di-alkylamino, e.g., R is ((dimethylamino)ethyl)amino

In certain embodiments, Q is —NHC(═CHR⁹)N(R)₂, in which R⁹ is NO₂, CN, C₁₋₆ alkyl, —S(O)₂N(R)₂, —OR, —S(O)₂R, or H. For example, Q is —NHC(═CHR⁹)N(CH₃)₂, —NHC(═CHR⁹)NHCH₃, or —NHC(═CHR⁹)NH₂.

In certain embodiments, Q is —OC(O)N(R)₂, —N(R)C(O)OR, —N(OR)C(O)OR, such as —OC(O)NHCH₃, —N(OH)C(O)OCH₃, —N(OH)C(O)CH₃, —N(OCH₃)C(O)OCH₃, —N(OCH₃)C(O)CH₃, —N(OH)S(O)₂CH₃, or —NHC(O)OCH₃.

In certain embodiments, Q is —N(R)C(O)R, in which R is alkyl optionally substituted with C₁₋₃ alkoxyl or S(O)_{z}C₁₋₃ alkyl, in which z is 0, 1, or 2.

In certain embodiments, Q is an unsubstituted or substituted C₆₋₁₀ aryl (such as phenyl) or C₃₋₆ cycloalkyl.

In some embodiments, n is 1. In other embodiments, n is 2. In further embodiments, n is 3. In certain other embodiments, n is 4. For example, R⁴ may be —(CH₂)₂₀H. For example, R⁴ may be —(CH₂)₃OH. For example, R⁴ may be —(CH₂)₄₀H. For example, R⁴ may be benzyl. For example, R⁴ may be 4-methoxybenzyl.

In some embodiments, R⁴ is a C₃₋₆ carbocycle. In some embodiments, R⁴ is a C₃₋₆ cycloalkyl. For example, R⁴ may be cyclohexyl optionally substituted with e.g., OH, halo, C₁₋₆ alkyl, etc. For example, R⁴ may be 2-hydroxycyclohexyl.

In some embodiments, R is H.

In some embodiments, R is C₁₋₃ alkyl substituted with mono- or di-alkylamino, e.g., R is ((dimethylamino)ethyl)amino.

In some embodiments, R is C₁₋₆ alkyl substituted with one or more substituents selected from the group consisting of C₁₋₃ alkoxyl, amino, and C₁-C₃ dialkylamino.

In some embodiments, R is unsubstituted C₁₋₃ alkyl or unsubstituted C₂₋₃ alkenyl. For example, R⁴ may be —CH₂CH(OH)CH₃, —CH(CH₃)CH₂OH, or —CH₂CH(OH)CH₂CH₃.

In some embodiments, R is substituted C₁₋₃ alkyl, e.g., CH₂OH. For example, R⁴ may be —CH₂CH(OH)CH₂OH, —(CH₂)₃NHC(O)CH₂OH, —(CH₂)₃NHC(O)CH₂OBn, —(CH₂)₂₀(CH₂)₂₀H, —(CH₂)₃NHCH₂OCH₃, —(CH₂)₃NHCH₂OCH₂CH₃, CH₂SCH₃, CH₂S(O)CH₃, CH₂S(O)₂CH₃, or —CH(CH₂OH)₂.

In some embodiments, R⁴ is selected from any of the following groups:

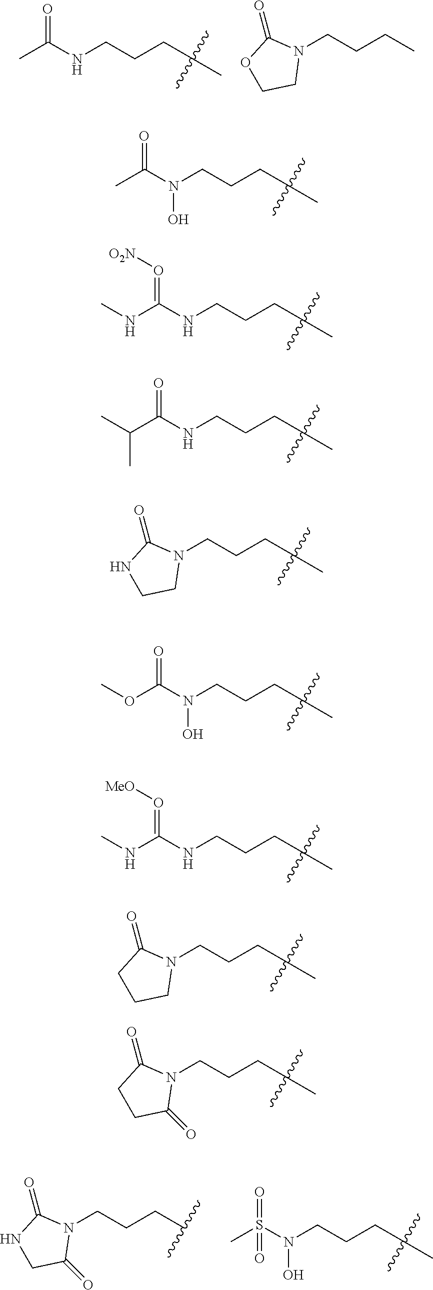

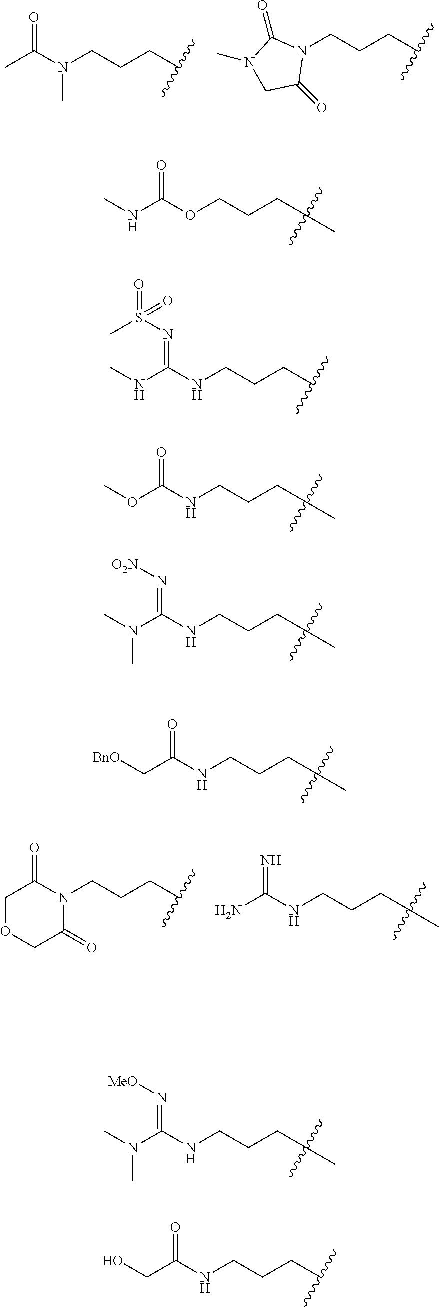

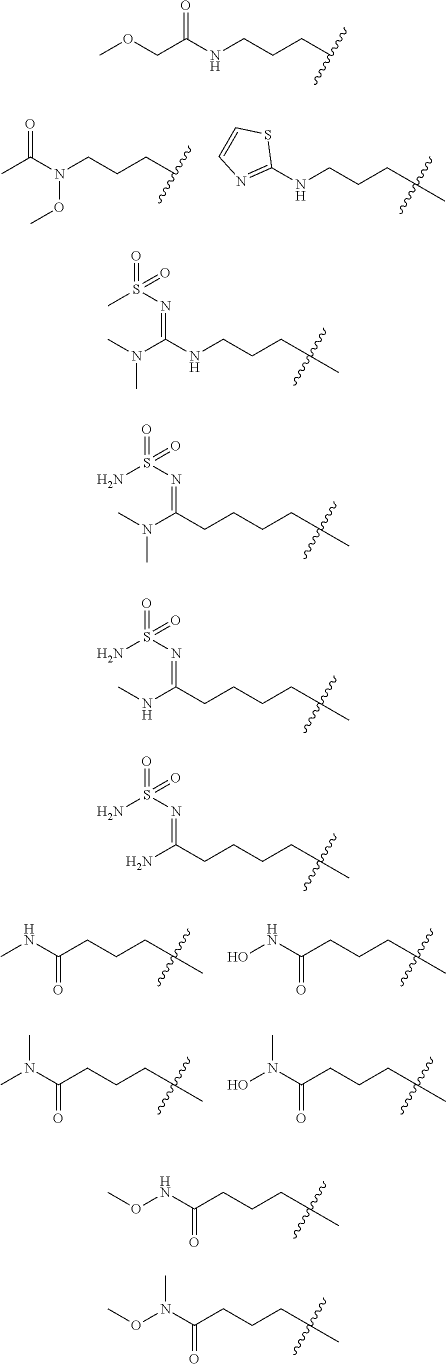

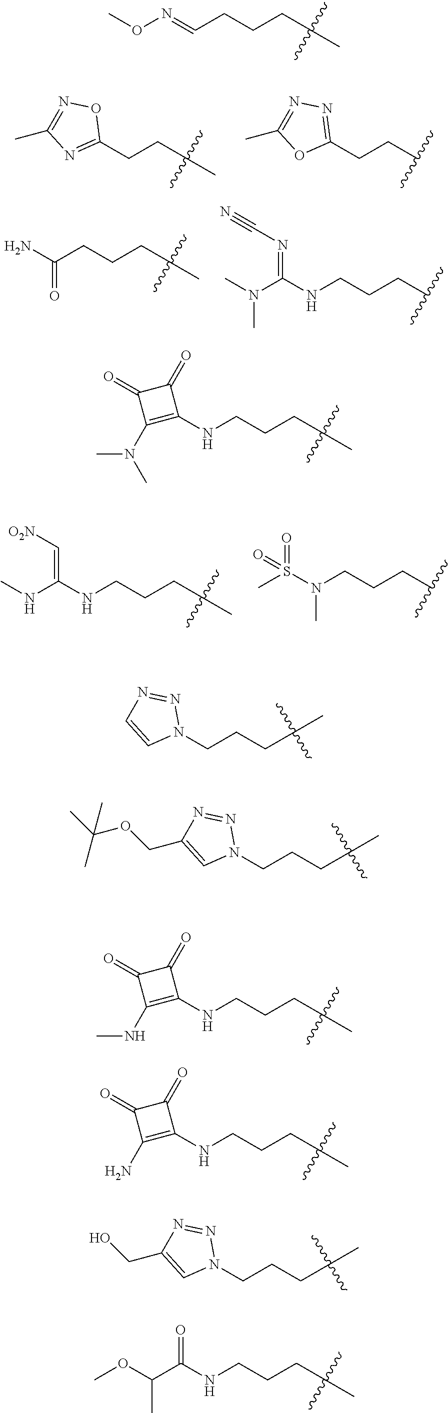

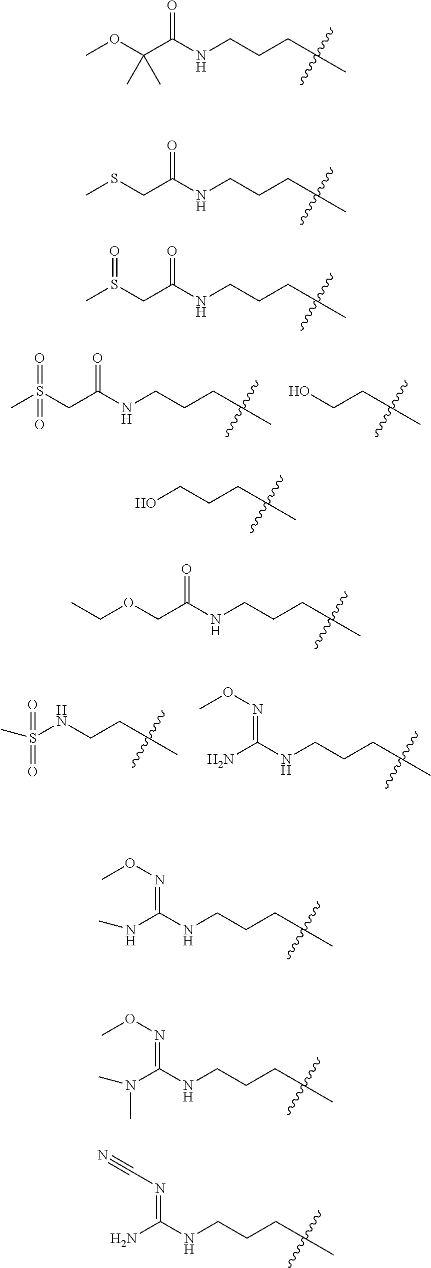

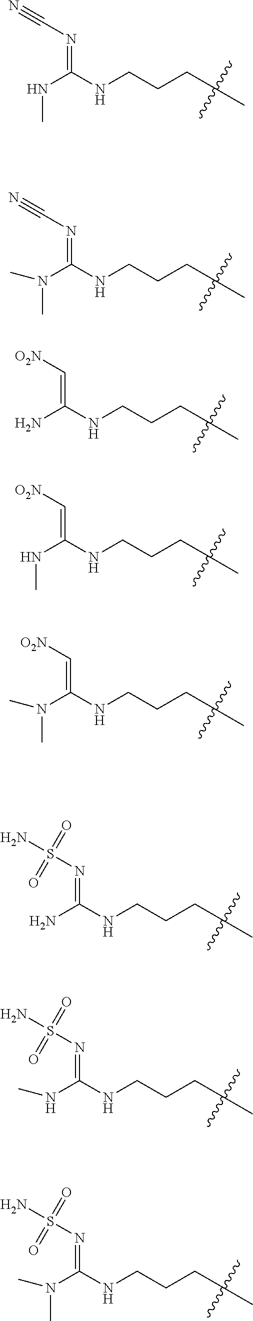

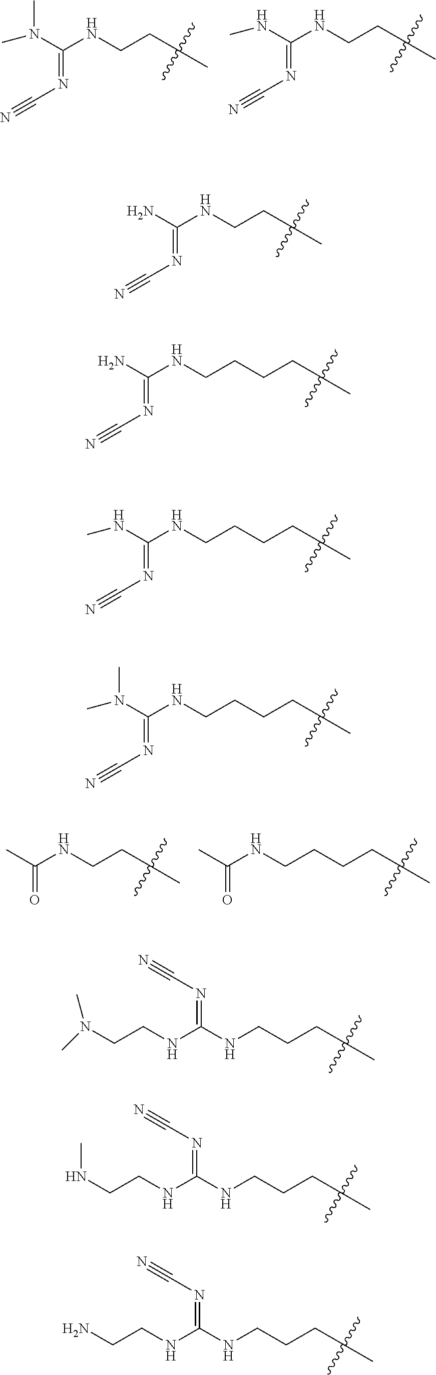

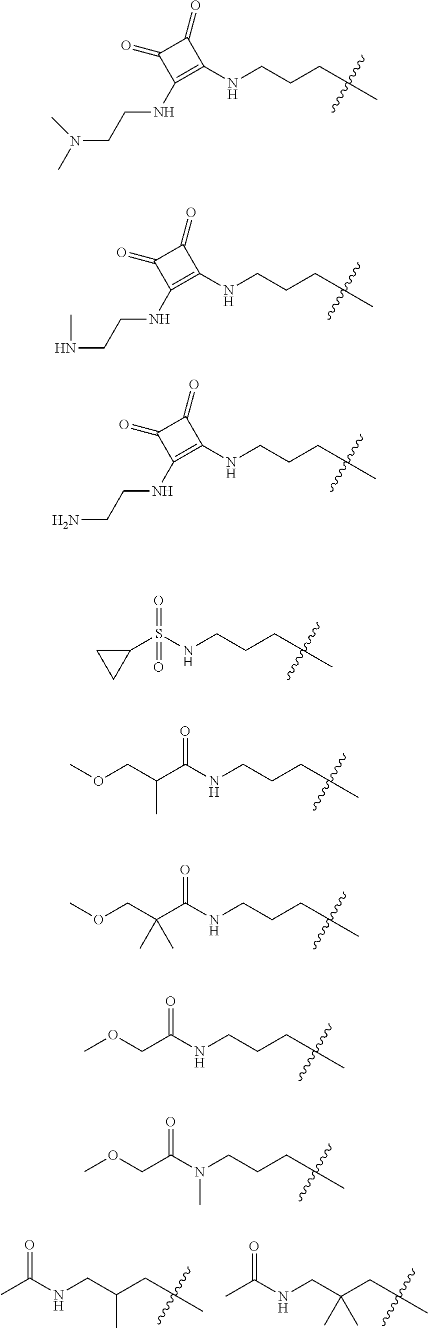

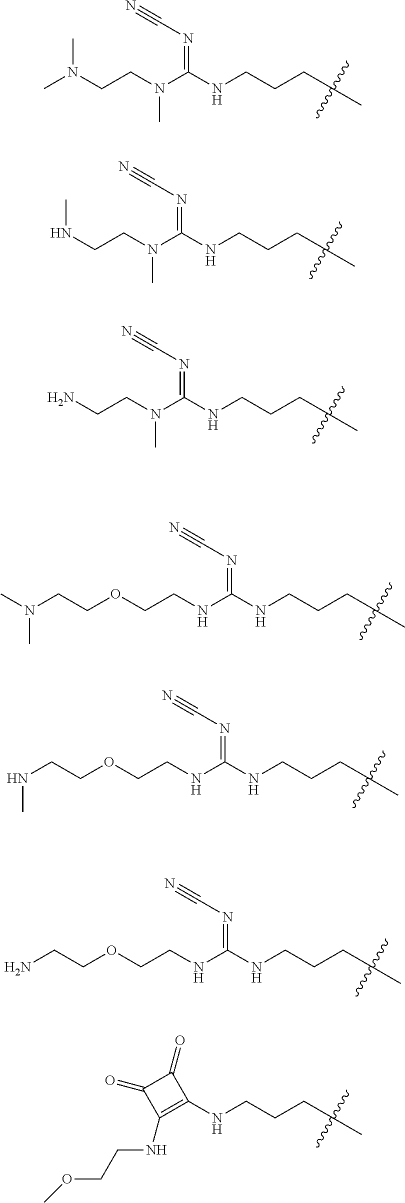

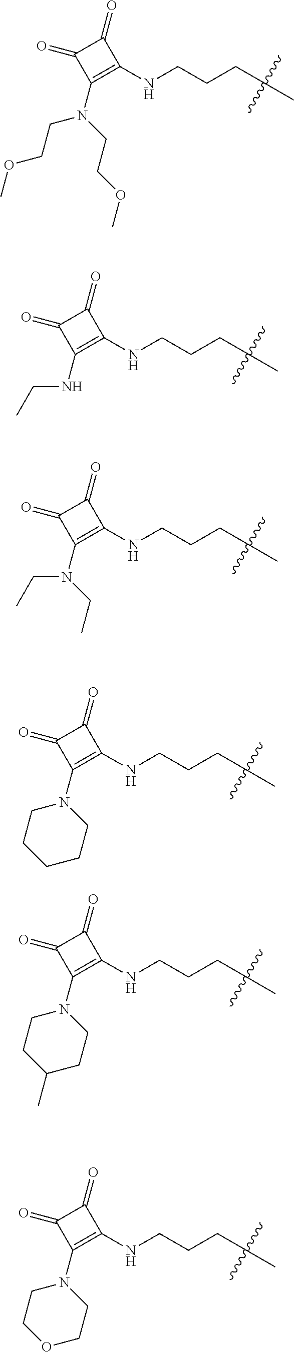

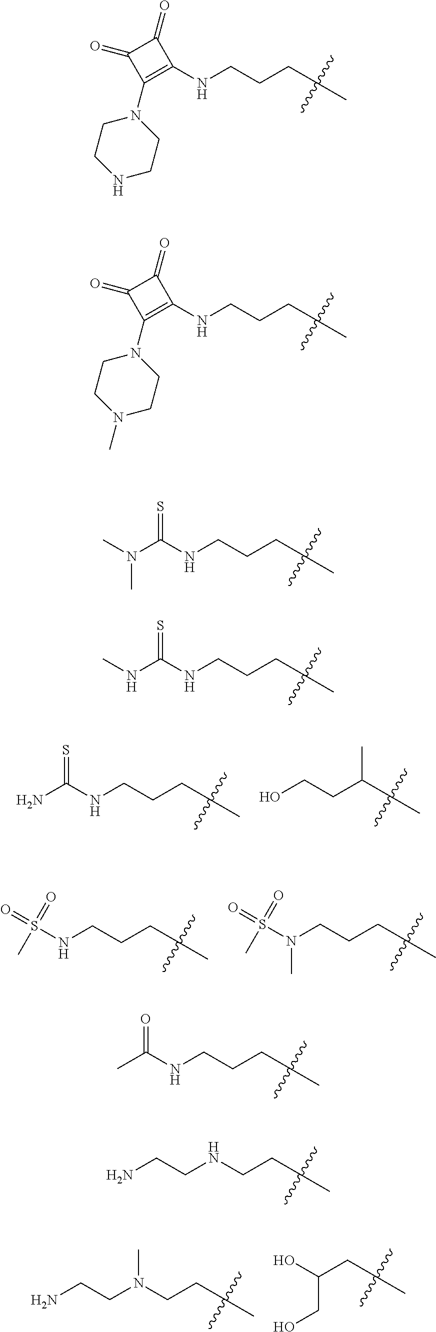

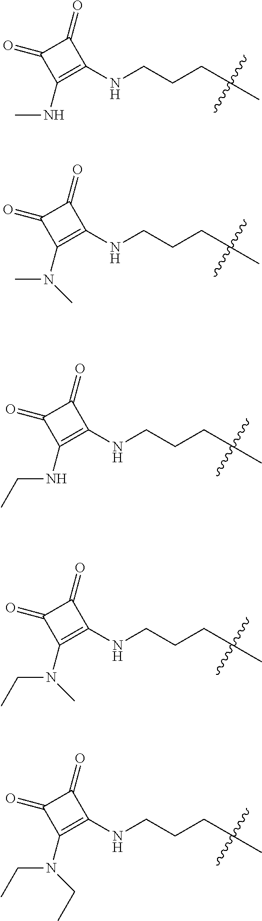 [SMILES: *CCCNc1c(NC)c(=O)c1=O ; *CCCNc1c(N(C)C)c(=O)c1=O ; *CCCNc1c(NCC)c(=O)c1=O ; *CCCNc1c(N(C)CC)c(=O)c1=O ; *CCCNc1c(N(CC)CC)c(=O)c1=O]

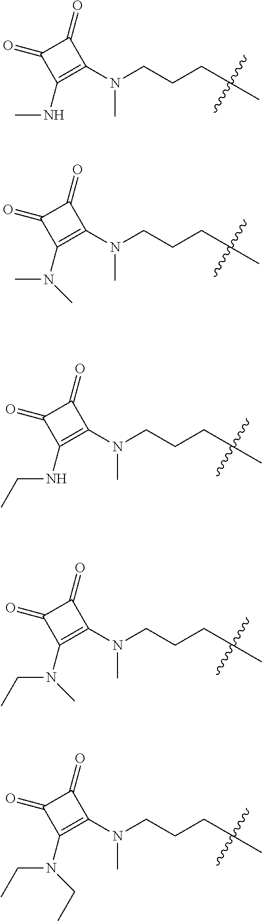 [SMILES: *CCCN(C)c1c(NC)c(=O)c1=O ; *CCCN(C)c1c(N(C)C)c(=O)c1=O ; *CCCN(C)c1c(NCC)c(=O)c1=O ; *CCCN(C)c1c(N(C)CC)c(=O)c1=O ; *CCCN(C)c1c(N(CC)CC)c(=O)c1=O]

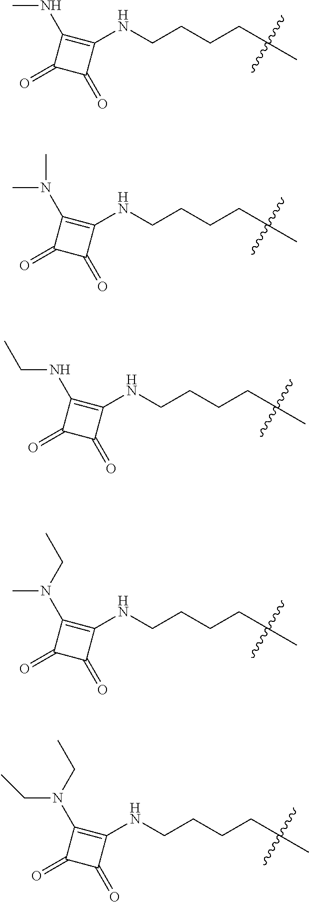 [SMILES: *CCCCNc1c(NC)c(=O)c1=O ; *CCCCNc1c(N(C)C)c(=O)c1=O ; *CCCCNc1c(NCC)c(=O)c1=O ; *CCCCNc1c(N(C)CC)c(=O)c1=O ; *CCCCNc1c(N(CC)CC)c(=O)c1=O]

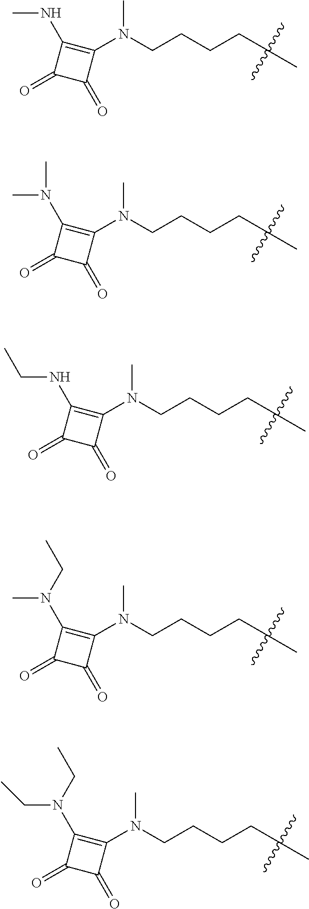 [SMILES: *CCCCN(C)c1c(NC)c(=O)c1=O ; *CCCCN(C)c1c(N(C)C)c(=O)c1=O ; *CCCCN(C)c1c(NCC)c(=O)c1=O ; *CCCCN(C)c1c(N(C)CC)c(=O)c1=O ; *CCCCN(C)c1c(N(CC)CC)c(=O)c1=O]

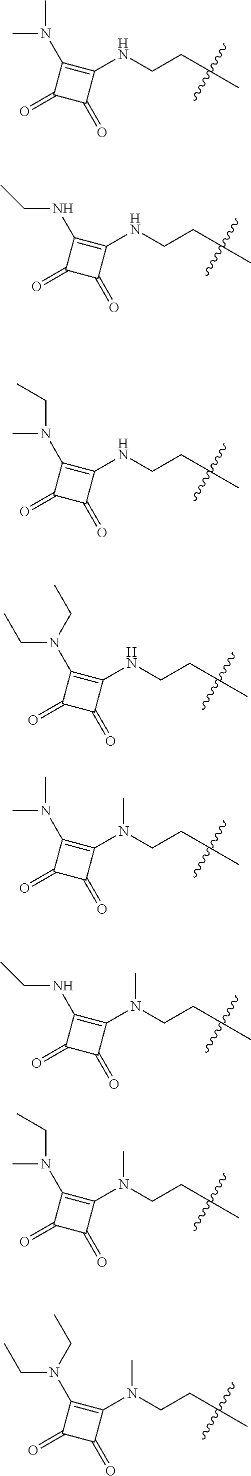

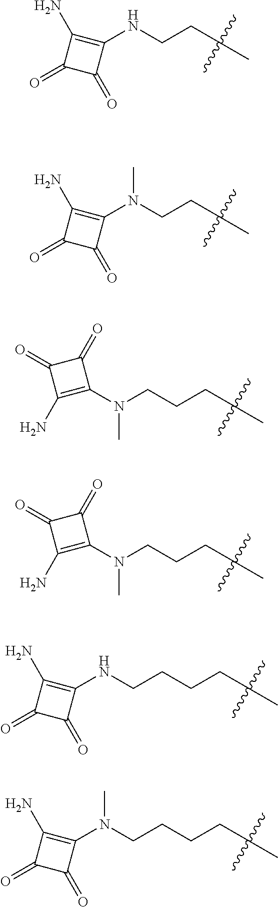

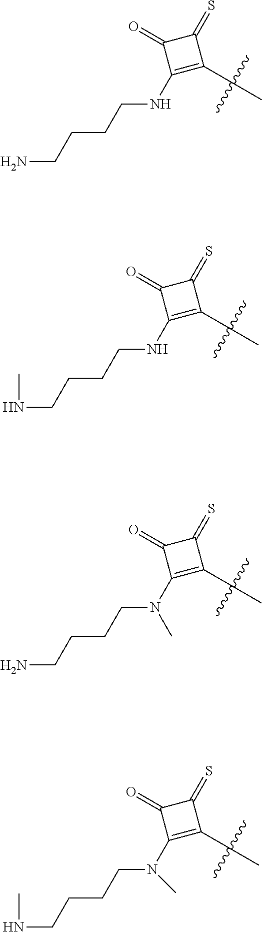 [SMILES: *c1c(NCCCCN)c(=O)c1=S ; *c1c(NCCCCNC)c(=O)c1=S ; *c1c(N(C)CCCCN)c(=O)c1=S ; *c1c(N(C)CCCCNC)c(=O)c1=S]

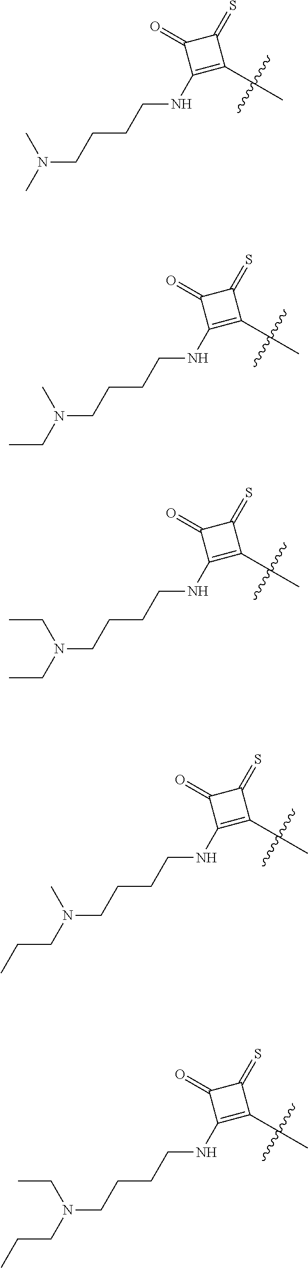 [SMILES: *c1c(NCCCCN(C)C)c(=O)c1=S ; *c1c(NCCCCN(C)CC)c(=O)c1=S ; *c1c(NCCCCN(CC)CC)c(=O)c1=S ; *c1c(NCCCCN(C)CCC)c(=O)c1=S ; *c1c(NCCCCN(CC)CCC)c(=O)c1=S]

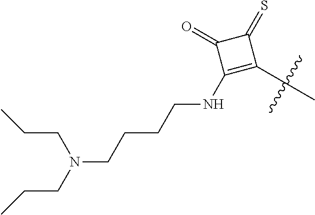 [SMILES: *c1c(NCCCCN(CCC)CCC)c(=O)c1=S]

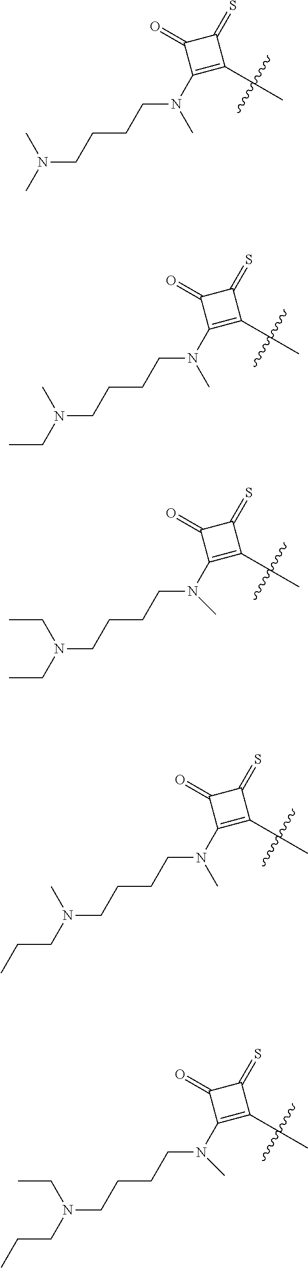 [SMILES: *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S ; *c1c(N(C)CCCCN(CC)CCC)c(=O)c1=S]

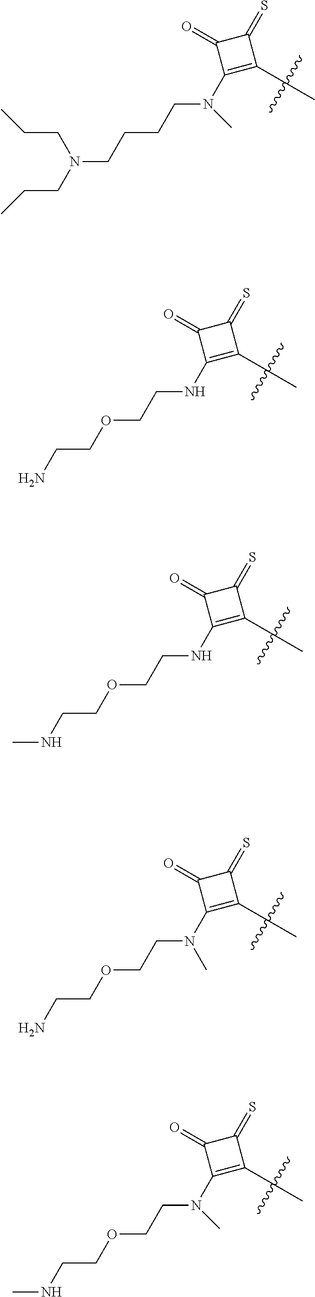 [SMILES: *c1c(N(C)CCCCN(CCC)CCC)c(=O)c1=S ; *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S]

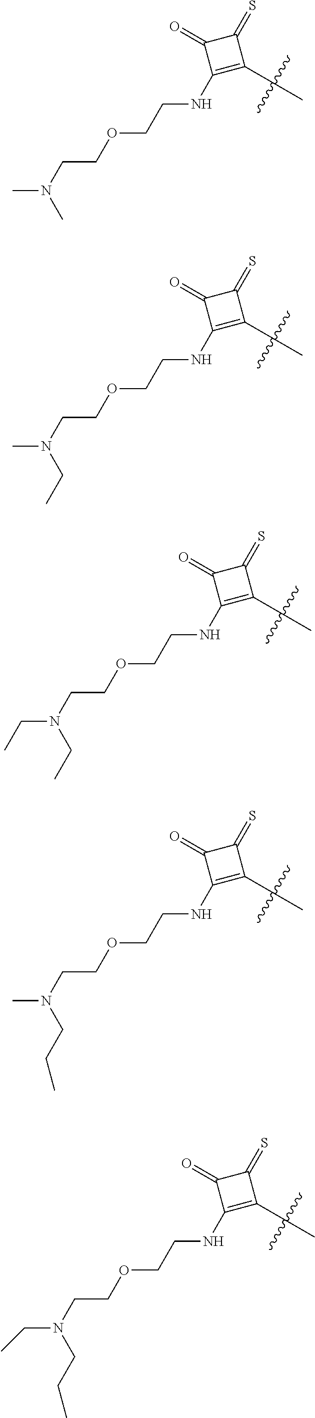 [SMILES: *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S ; *c1c(NCCOCCN(CC)CCC)c(=O)c1=S]

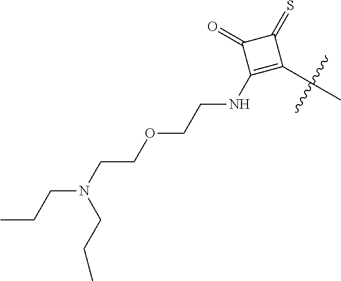 [SMILES: *c1c(NCCOCCN(CCC)CCC)c(=O)c1=S]

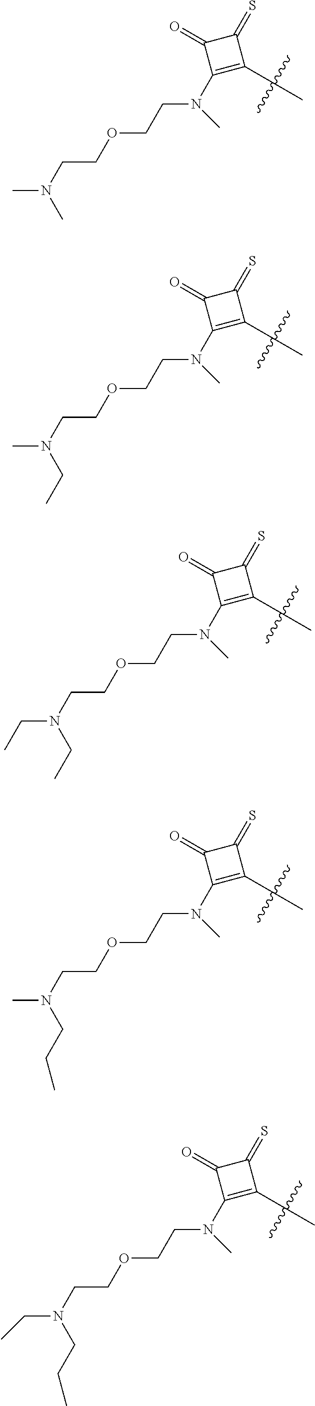 [SMILES: *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S ; *c1c(N(C)CCOCCN(CC)CCC)c(=O)c1=S]

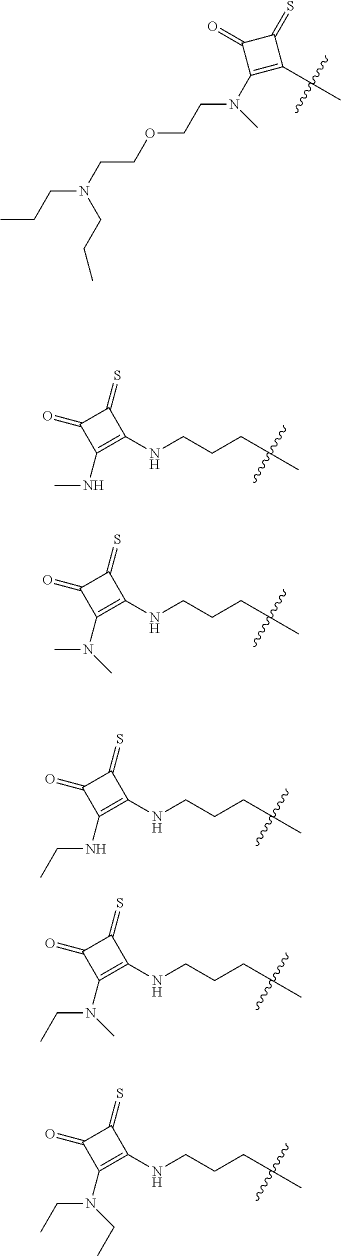 [SMILES: *c1c(N(C)CCOCCN(CCC)CCC)c(=O)c1=S ; *CCCNc1c(NC)c(=O)c1=S ; *CCCNc1c(N(C)C)c(=O)c1=S ; *CCCNc1c(NCC)c(=O)c1=S ; *CCCNc1c(N(C)CC)c(=O)c1=S ; *CCCNc1c(N(CC)CC)c(=O)c1=S]

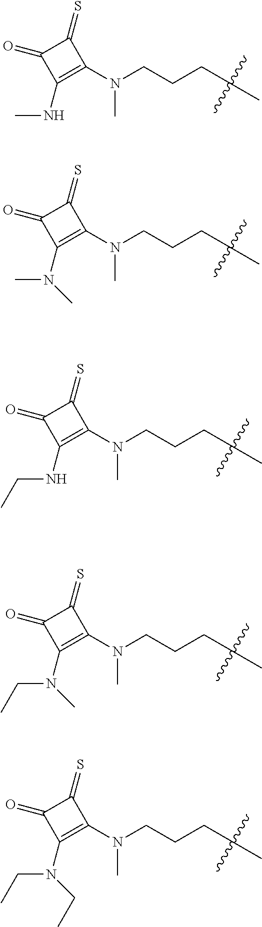 [SMILES: *CCCN(C)c1c(NC)c(=O)c1=S ; *CCCN(C)c1c(N(C)C)c(=O)c1=S ; *CCCN(C)c1c(NCC)c(=O)c1=S ; *CCCN(C)c1c(N(C)CC)c(=O)c1=S ; *CCCN(C)c1c(N(CC)CC)c(=O)c1=S]

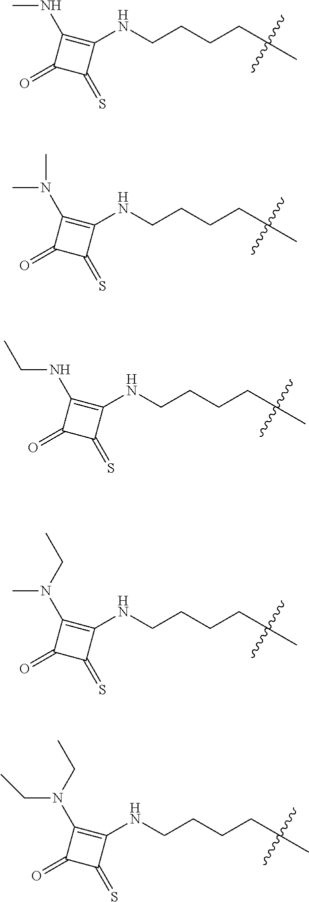 [SMILES: *CCCCNc1c(NC)c(=O)c1=S ; *CCCCNc1c(N(C)C)c(=O)c1=S ; *CCCCNc1c(NCC)c(=O)c1=S ; *CCCCNc1c(N(C)CC)c(=O)c1=S ; *CCCCNc1c(N(CC)CC)c(=O)c1=S]

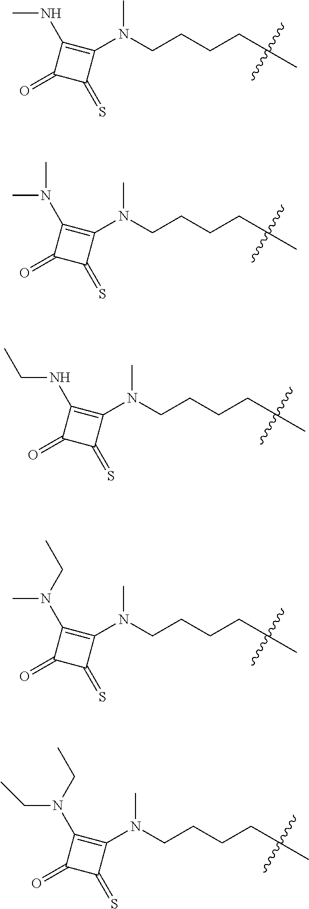 [SMILES: *CCCCN(C)c1c(NC)c(=O)c1=S ; *CCCCN(C)c1c(N(C)C)c(=O)c1=S ; *CCCCN(C)c1c(NCC)c(=O)c1=S ; *CCCCN(C)c1c(N(C)CC)c(=O)c1=S ; *CCCCN(C)c1c(N(CC)CC)c(=O)c1=S]

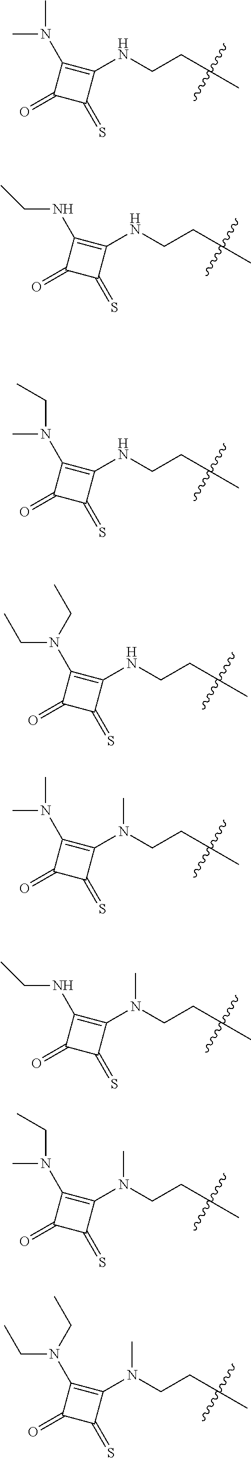

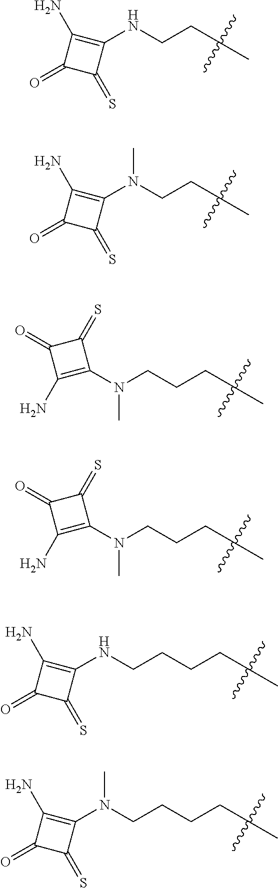

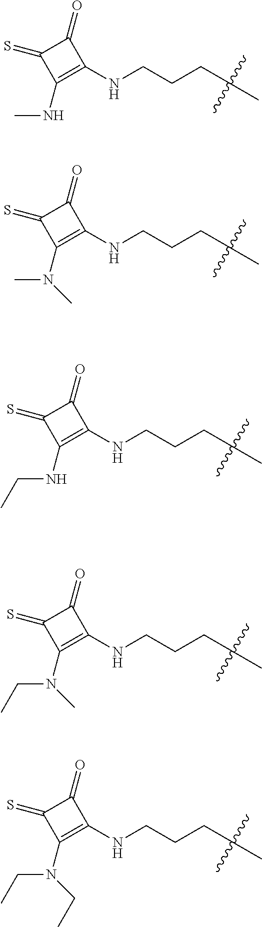 [SMILES: *CCCNc1c(NC)c(=S)c1=O ; *CCCNc1c(N(C)C)c(=S)c1=O ; *CCCNc1c(NCC)c(=S)c1=O ; *CCCNc1c(N(C)CC)c(=S)c1=O ; *CCCNc1c(N(CC)CC)c(=S)c1=O]

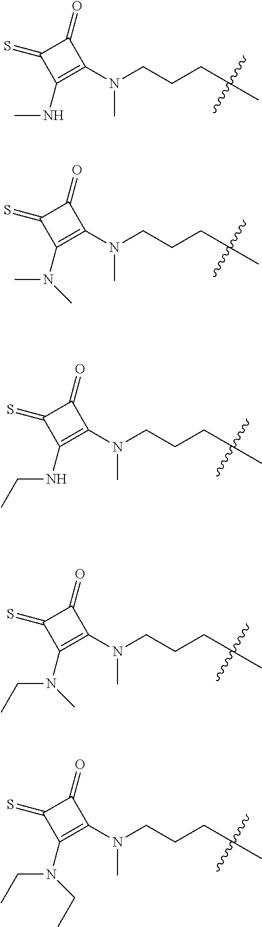 [SMILES: *CCCN(C)c1c(NC)c(=S)c1=O ; *CCCN(C)c1c(N(C)C)c(=S)c1=O ; *CCCN(C)c1c(NCC)c(=S)c1=O ; *CCCN(C)c1c(N(C)CC)c(=S)c1=O ; *CCCN(C)c1c(N(CC)CC)c(=S)c1=O]

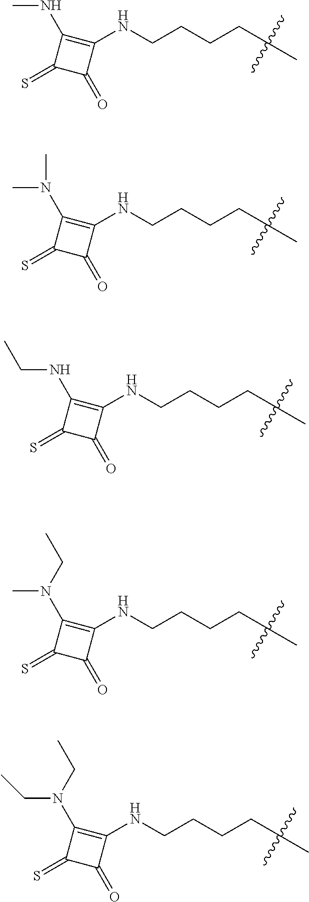 [SMILES: *CCCCNc1c(NC)c(=S)c1=O ; *CCCCNc1c(N(C)C)c(=S)c1=O ; *CCCCNc1c(NCC)c(=S)c1=O ; *CCCCNc1c(N(C)CC)c(=S)c1=O ; *CCCCNc1c(N(CC)CC)c(=S)c1=O]

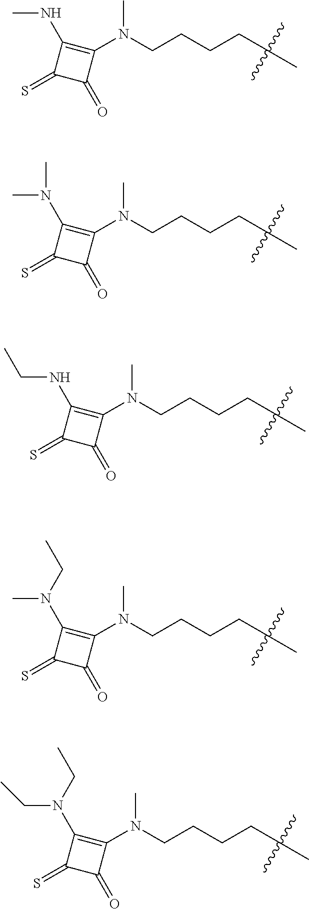 [SMILES: *CCCCN(C)c1c(NC)c(=S)c1=O ; *CCCCN(C)c1c(N(C)C)c(=S)c1=O ; *CCCCN(C)c1c(NCC)c(=S)c1=O ; *CCCCN(C)c1c(N(C)CC)c(=S)c1=O ; *CCCCN(C)c1c(N(CC)CC)c(=S)c1=O]

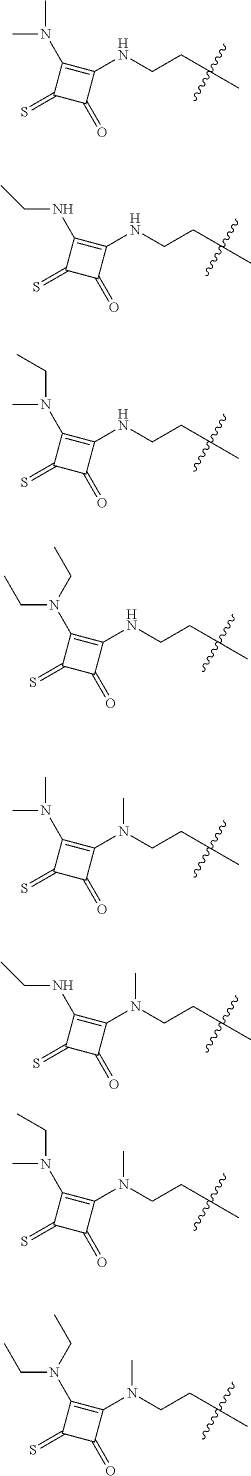

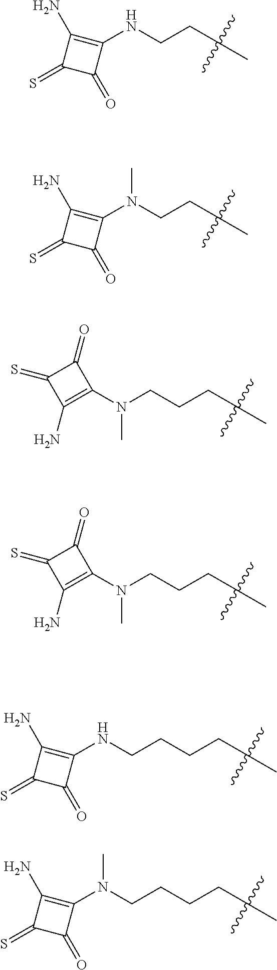

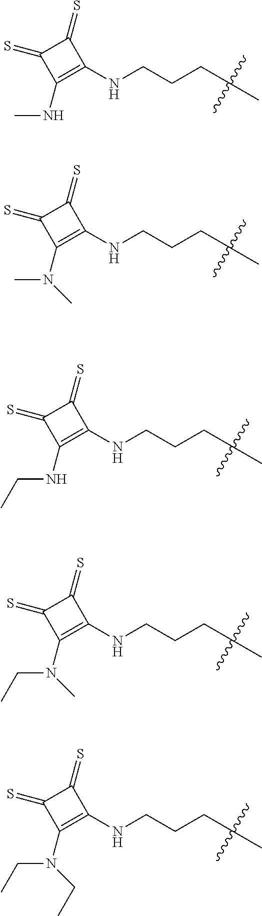 [SMILES: *CCCNc1c(NC)c(=S)c1=S ; *CCCNc1c(N(C)C)c(=S)c1=S ; *CCCNc1c(NCC)c(=S)c1=S ; *CCCNc1c(N(C)CC)c(=S)c1=S ; *CCCNc1c(N(CC)CC)c(=S)c1=S]

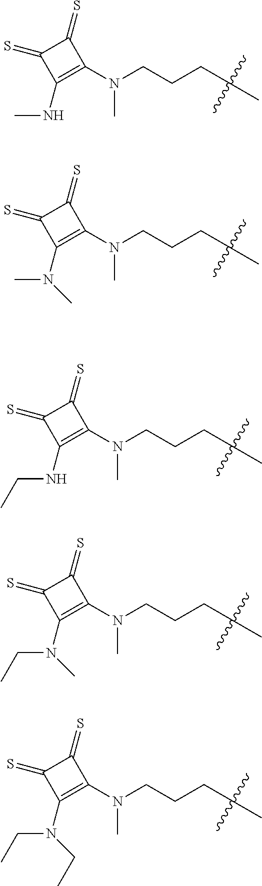 [SMILES: *CCCN(C)c1c(NC)c(=S)c1=S ; *CCCN(C)c1c(N(C)C)c(=S)c1=S ; *CCCN(C)c1c(NCC)c(=S)c1=S ; *CCCN(C)c1c(N(C)CC)c(=S)c1=S ; *CCCN(C)c1c(N(CC)CC)c(=S)c1=S]

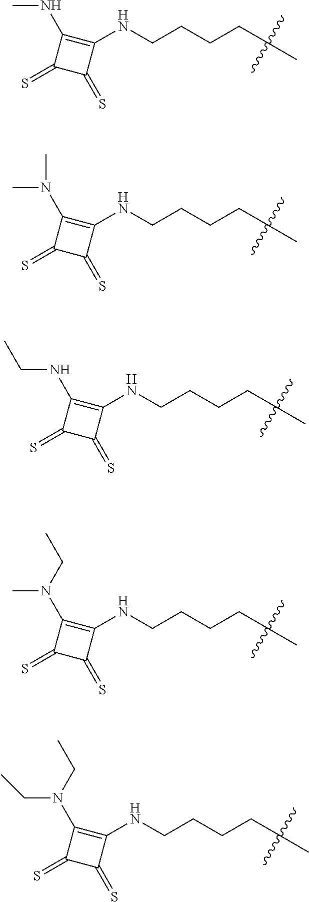 [SMILES: *CCCCNc1c(NC)c(=S)c1=S ; *CCCCNc1c(N(C)C)c(=S)c1=S ; *CCCCNc1c(NCC)c(=S)c1=S ; *CCCCNc1c(N(C)CC)c(=S)c1=S ; *CCCCNc1c(N(CC)CC)c(=S)c1=S]

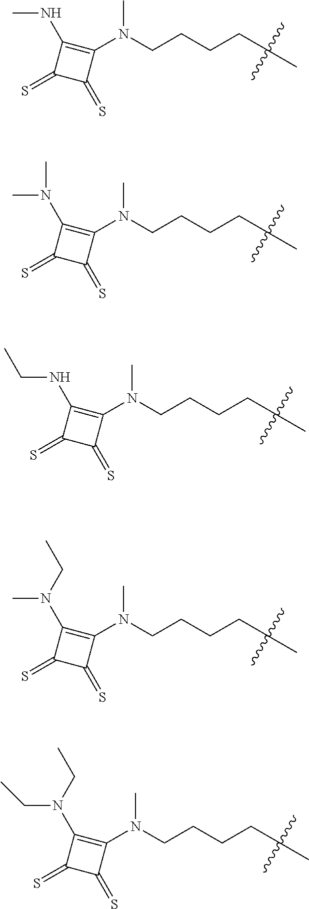 [SMILES: *CCCCN(C)c1c(NC)c(=S)c1=S ; *CCCCN(C)c1c(N(C)C)c(=S)c1=S ; *CCCCN(C)c1c(NCC)c(=S)c1=S ; *CCCCN(C)c1c(N(C)CC)c(=S)c1=S ; *CCCCN(C)c1c(N(CC)CC)c(=S)c1=S]

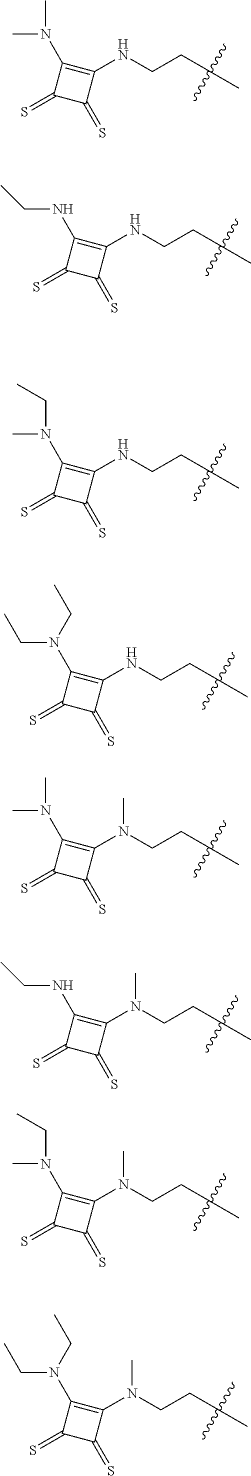

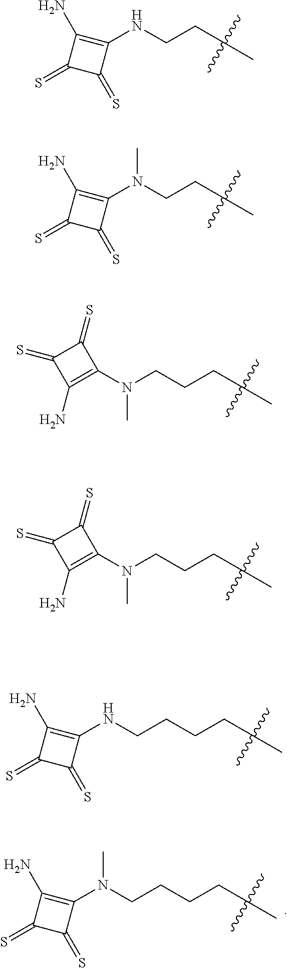 [SMILES: *CCNc1c(N)c(=S)c1=S ; *CCN(C)c1c(N)c(=S)c1=S ; *CCCN(C)c1c(N)c(=S)c1=S ; *CCCN(C)c1c(N)c(=S)c1=S ; *CCCCNc1c(N)c(=S)c1=S ; *CCCCN(C)c1c(N)c(=S)c1=S]

In some embodiments,

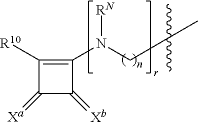

is selected from any of the following groups:

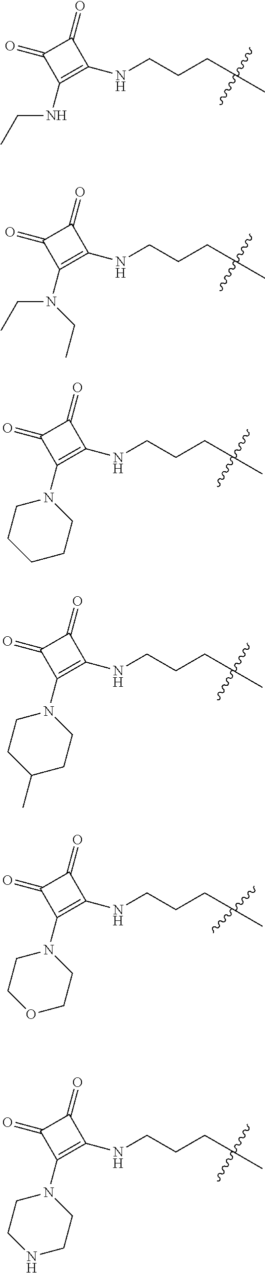

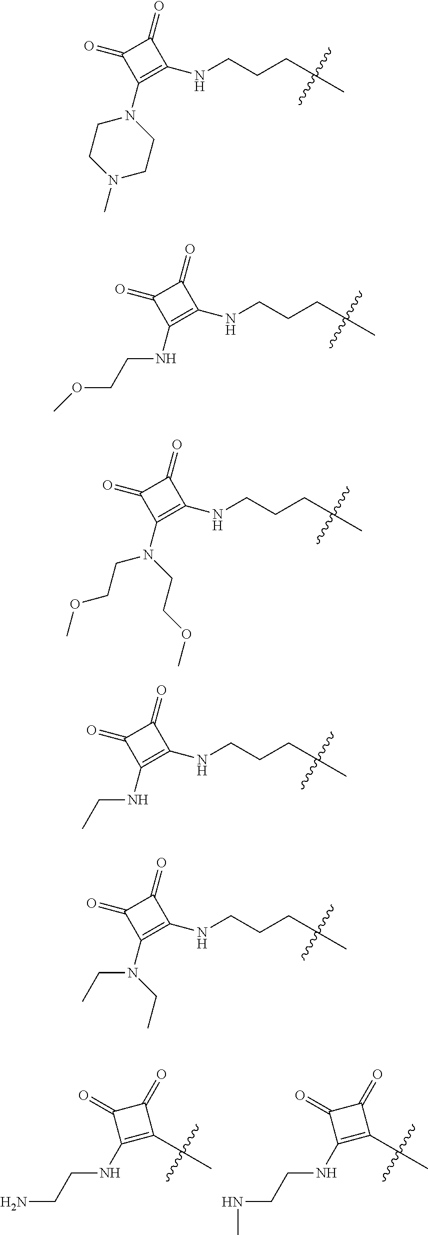

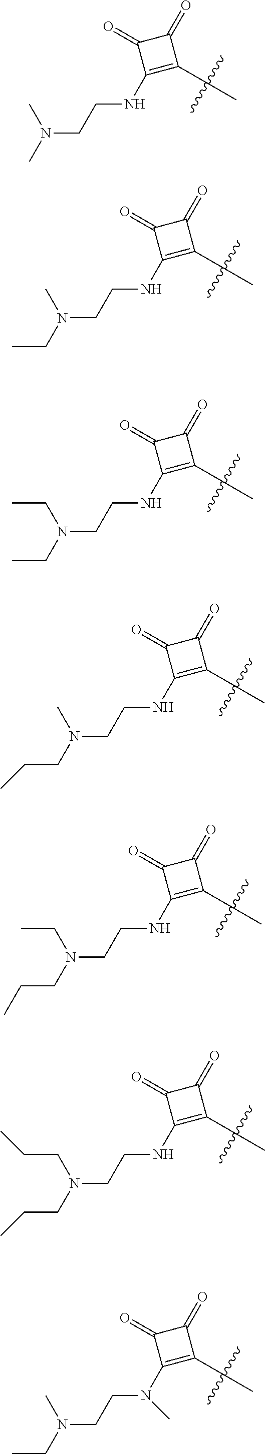

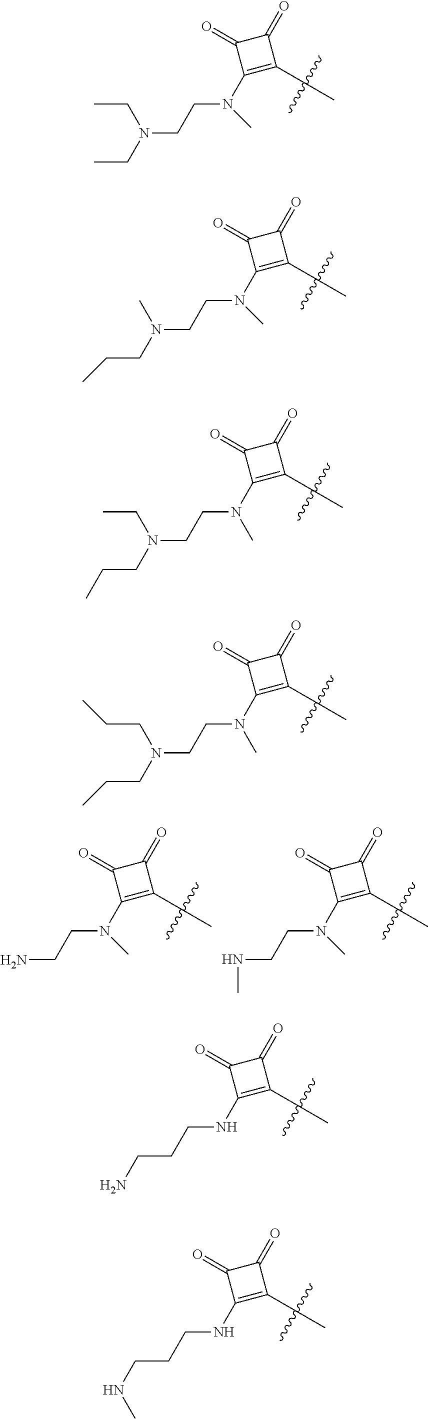

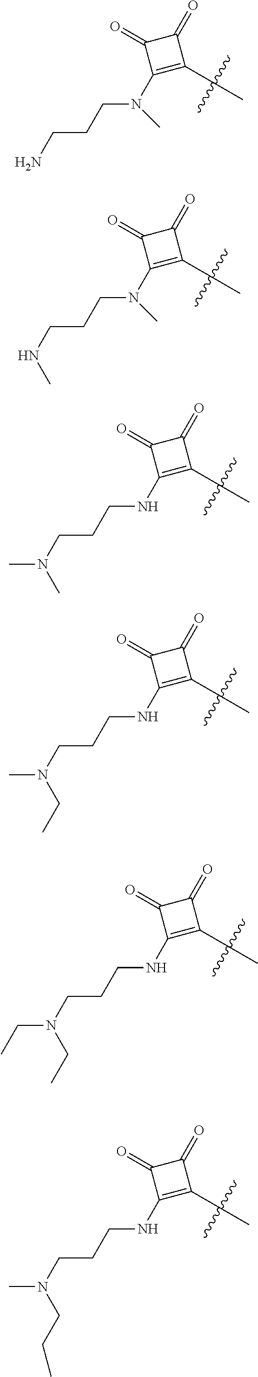 [SMILES: *c1c(N(C)CCCN)c(=O)c1=O ; *c1c(N(C)CCCNC)c(=O)c1=O ; *c1c(NCCCN(C)C)c(=O)c1=O ; *c1c(NCCCN(C)CC)c(=O)c1=O ; *c1c(NCCCN(CC)CC)c(=O)c1=O ; *c1c(NCCCN(C)CCC)c(=O)c1=O]

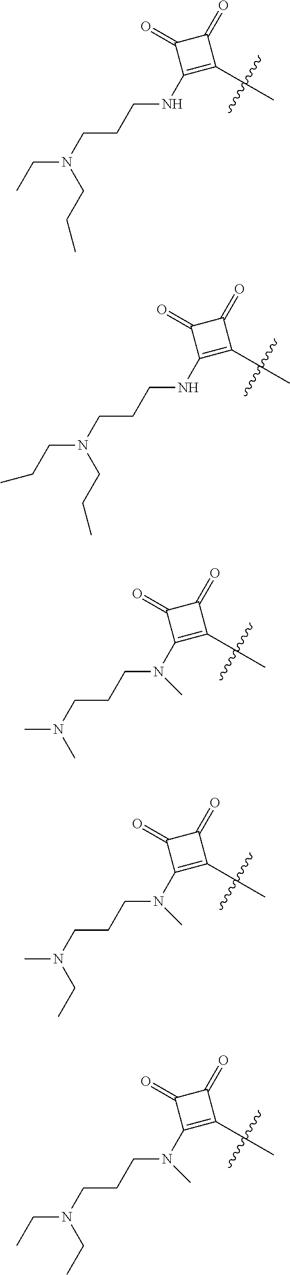 [SMILES: *c1c(NCCCN(CC)CCC)c(=O)c1=O ; *c1c(NCCCN(CCC)CCC)c(=O)c1=O ; *c1c(N(C)CCCN(C)C)c(=O)c1=O ; *c1c(N(C)CCCN(C)CC)c(=O)c1=O ; *c1c(N(C)CCCN(CC)CC)c(=O)c1=O]

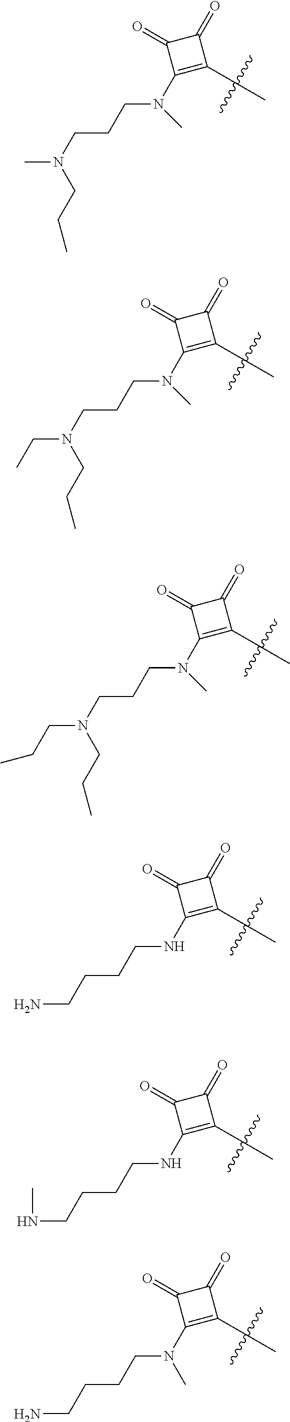 [SMILES: *c1c(N(C)CCCN(C)CCC)c(=O)c1=O ; *c1c(N(C)CCCN(CC)CCC)c(=O)c1=O ; *c1c(N(C)CCCN(CCC)CCC)c(=O)c1=O ; *c1c(NCCCCN)c(=O)c1=O ; *c1c(NCCCCNC)c(=O)c1=O ; *c1c(N(C)CCCCN)c(=O)c1=O]

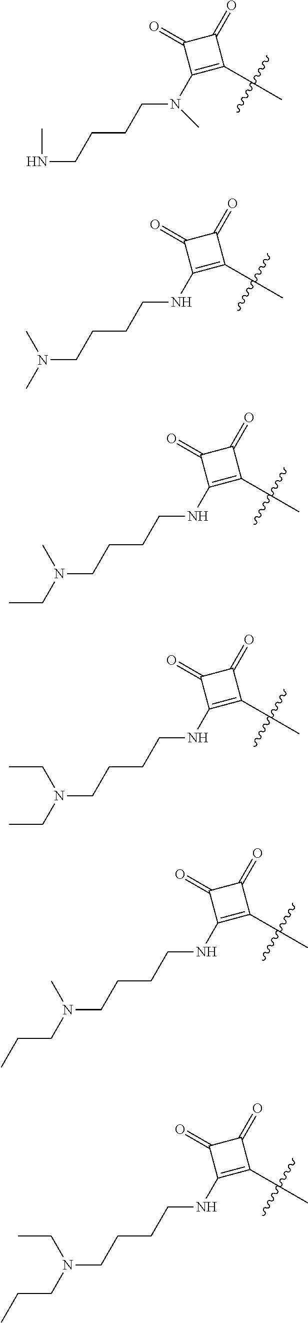 [SMILES: *c1c(N(C)CCCCNC)c(=O)c1=O ; *c1c(NCCCCN(C)C)c(=O)c1=O ; *c1c(NCCCCN(C)CC)c(=O)c1=O ; *c1c(NCCCCN(CC)CC)c(=O)c1=O ; *c1c(NCCCCN(C)CCC)c(=O)c1=O ; *c1c(NCCCCN(CC)CCC)c(=O)c1=O]

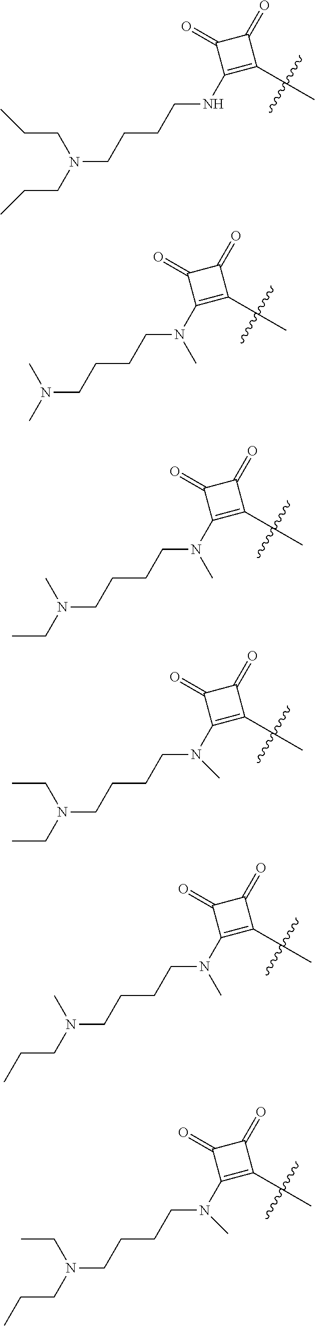 [SMILES: *c1c(NCCCCN(CCC)CCC)c(=O)c1=O ; *c1c(N(C)CCCCN(C)C)c(=O)c1=O ; *c1c(N(C)CCCCN(C)CC)c(=O)c1=O ; *c1c(N(C)CCCCN(CC)CC)c(=O)c1=O ; *c1c(N(C)CCCCN(C)CCC)c(=O)c1=O ; *c1c(N(C)CCCCN(CC)CCC)c(=O)c1=O]

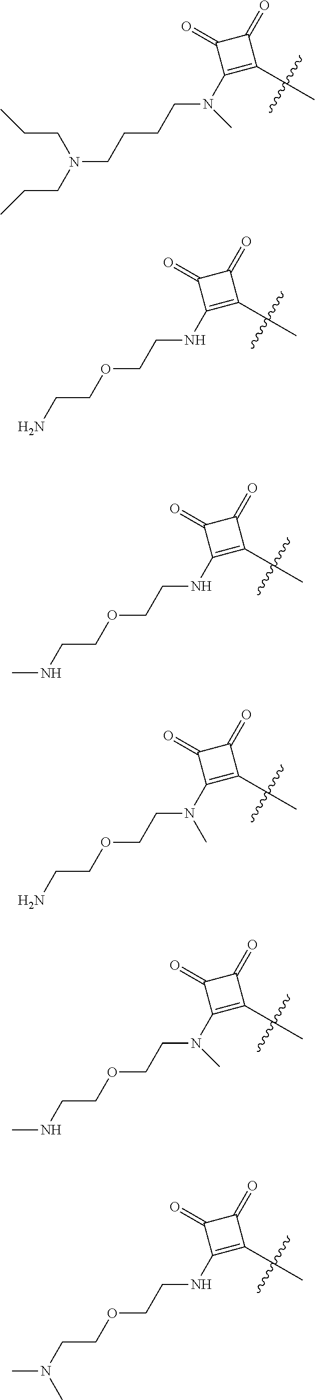 [SMILES: *c1c(N(C)CCCCN(CCC)CCC)c(=O)c1=O ; *c1c(NCCOCCN)c(=O)c1=O ; *c1c(NCCOCCNC)c(=O)c1=O ; *c1c(N(C)CCOCCN)c(=O)c1=O ; *c1c(N(C)CCOCCNC)c(=O)c1=O ; *c1c(NCCOCCN(C)C)c(=O)c1=O]

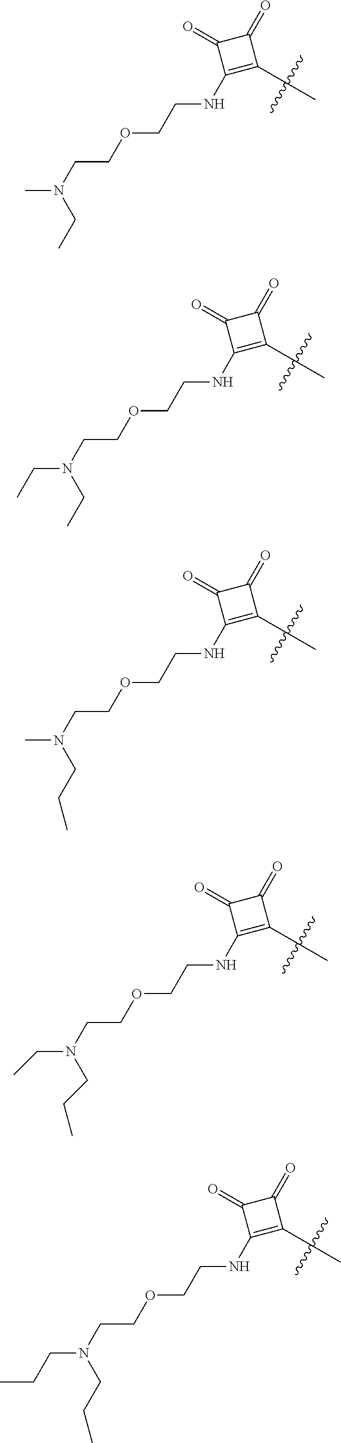 [SMILES: *c1c(NCCOCCN(C)CC)c(=O)c1=O ; *c1c(NCCOCCN(CC)CC)c(=O)c1=O ; *c1c(NCCOCCN(C)CCC)c(=O)c1=O ; *c1c(NCCOCCN(CC)CCC)c(=O)c1=O ; *c1c(NCCOCCN(CCC)CCC)c(=O)c1=O]

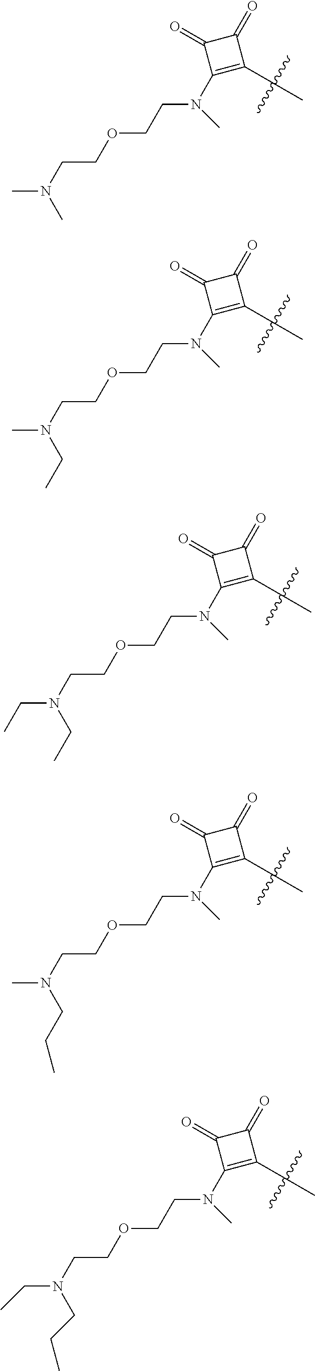 [SMILES: *c1c(N(C)CCOCCN(C)C)c(=O)c1=O ; *c1c(N(C)CCOCCN(C)CC)c(=O)c1=O ; *c1c(N(C)CCOCCN(CC)CC)c(=O)c1=O ; *c1c(N(C)CCOCCN(C)CCC)c(=O)c1=O ; *c1c(N(C)CCOCCN(CC)CCC)c(=O)c1=O]

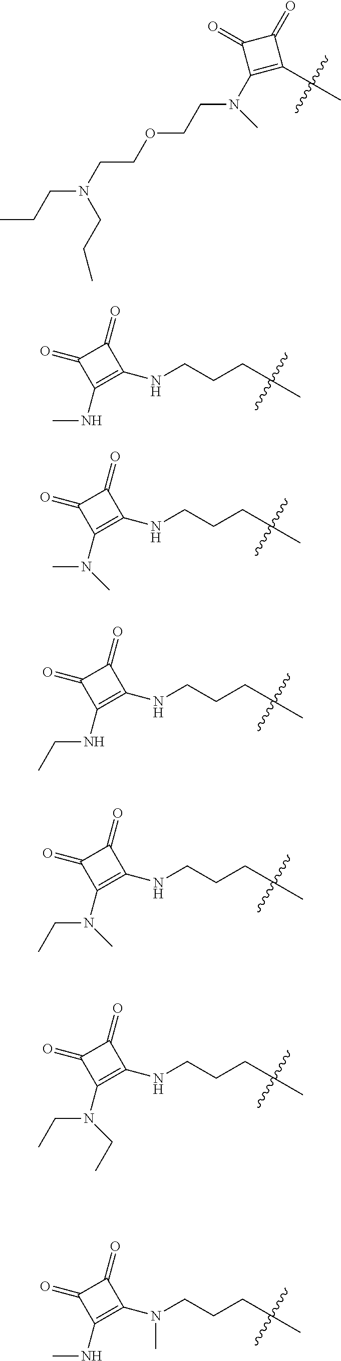

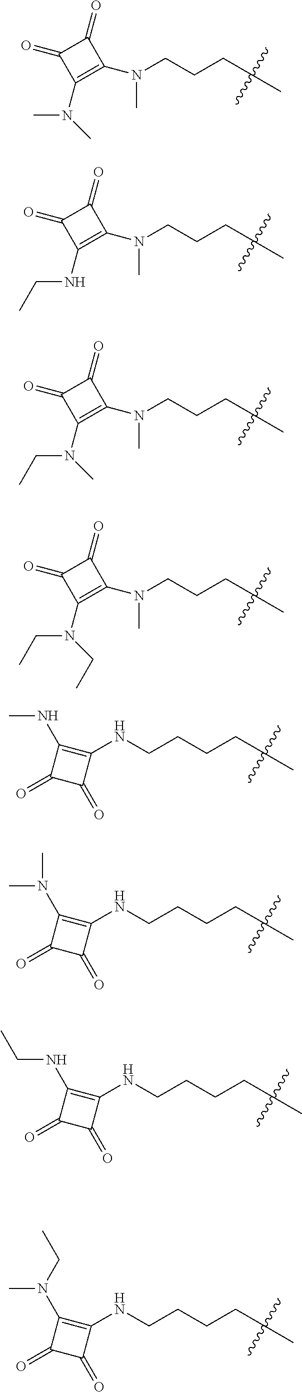

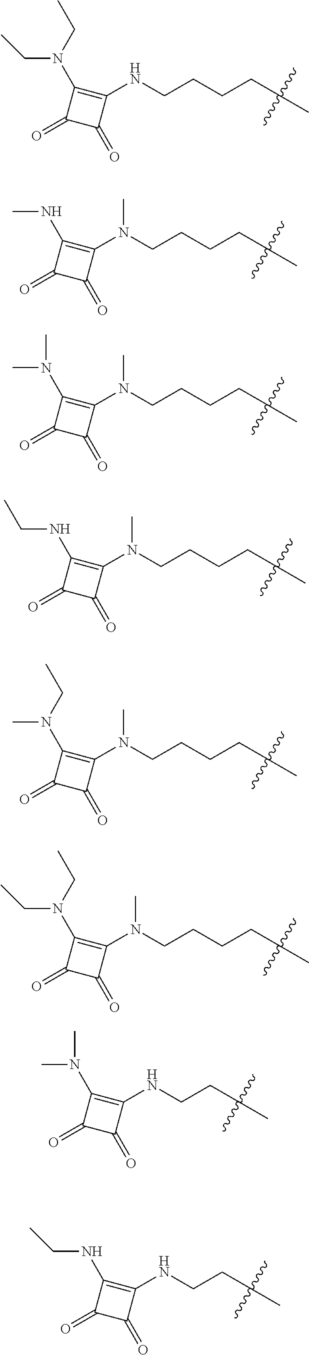

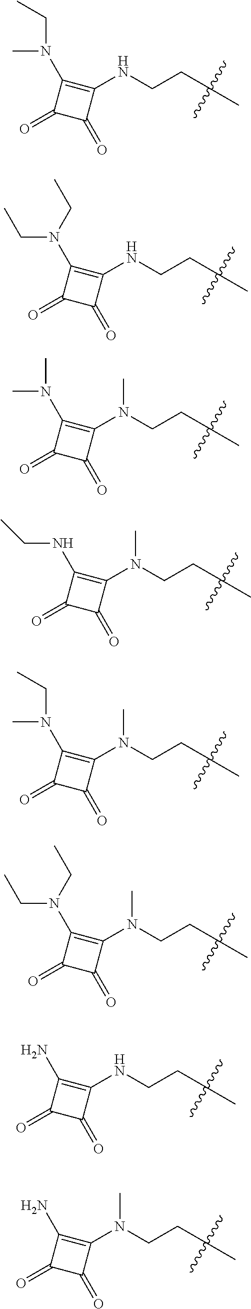

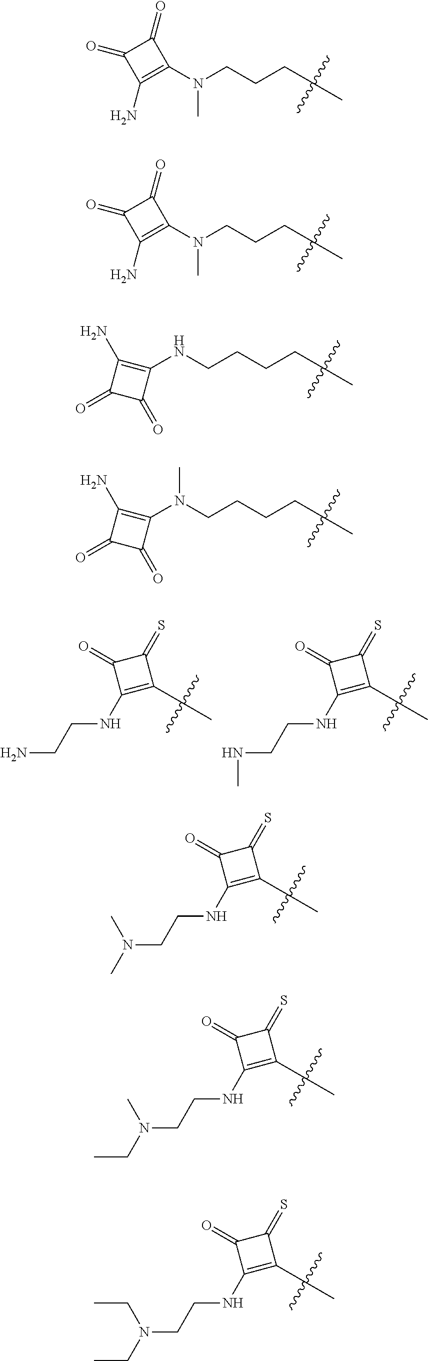

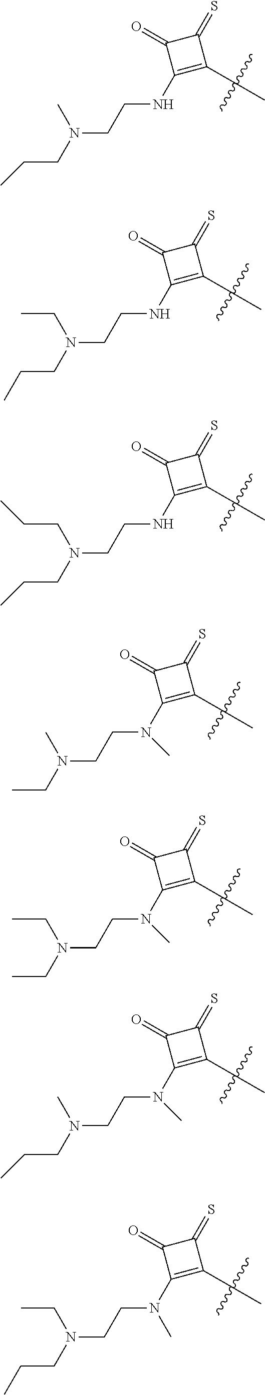

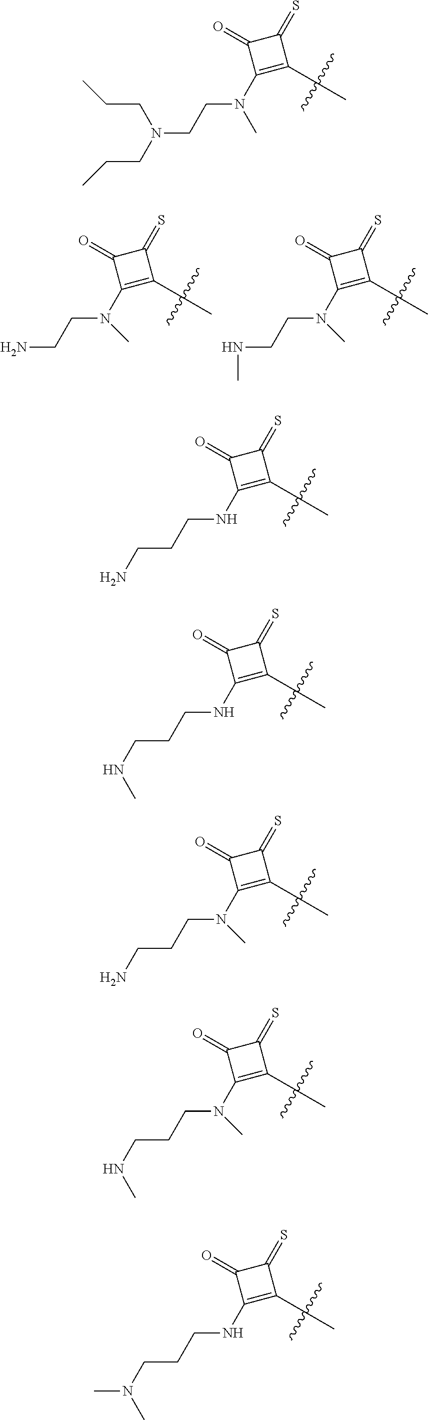

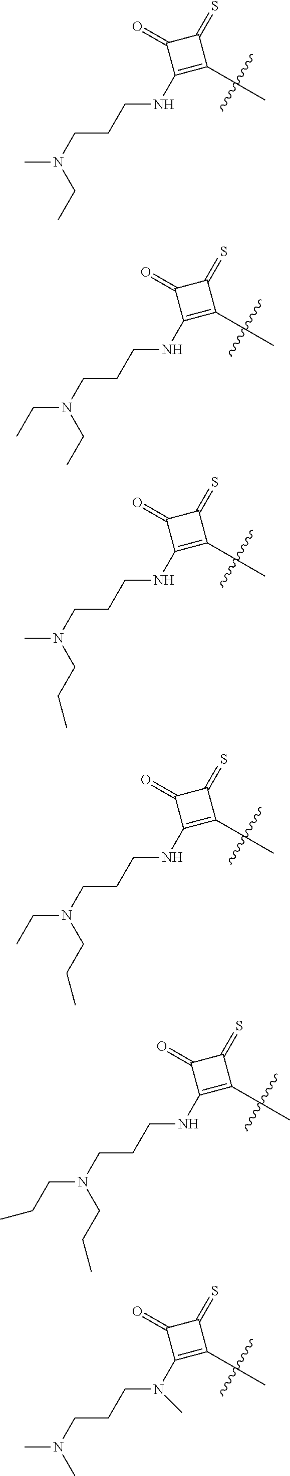 [SMILES: *c1c(NCCCN(C)CC)c(=O)c1=S ; *c1c(NCCCN(CC)CC)c(=O)c1=S ; *c1c(NCCCN(C)CCC)c(=O)c1=S ; *c1c(NCCCN(CC)CCC)c(=O)c1=S ; *c1c(NCCCN(CCC)CCC)c(=O)c1=S ; *c1c(N(C)CCCN(C)C)c(=O)c1=S]

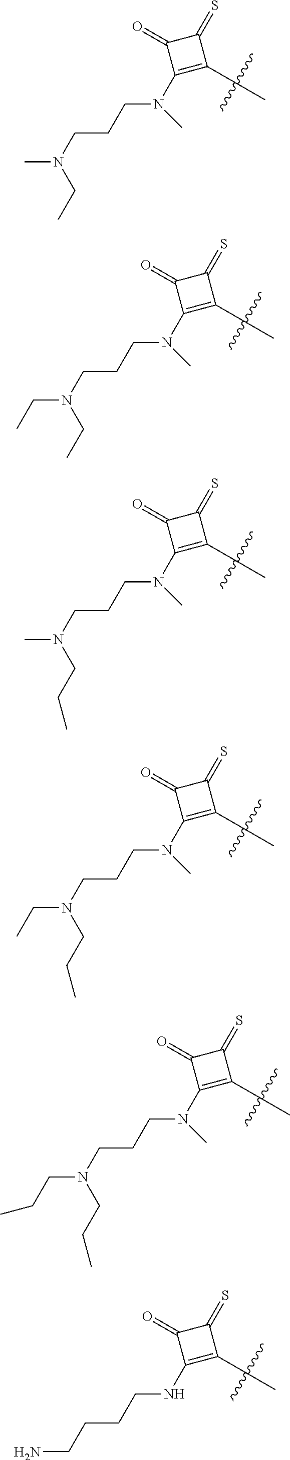 [SMILES: *c1c(N(C)CCCN(C)CC)c(=O)c1=S ; *c1c(N(C)CCCN(CC)CC)c(=O)c1=S ; *c1c(N(C)CCCN(C)CCC)c(=O)c1=S ; *c1c(N(C)CCCN(CC)CCC)c(=O)c1=S ; *c1c(N(C)CCCN(CCC)CCC)c(=O)c1=S ; *c1c(NCCCCN)c(=O)c1=S]

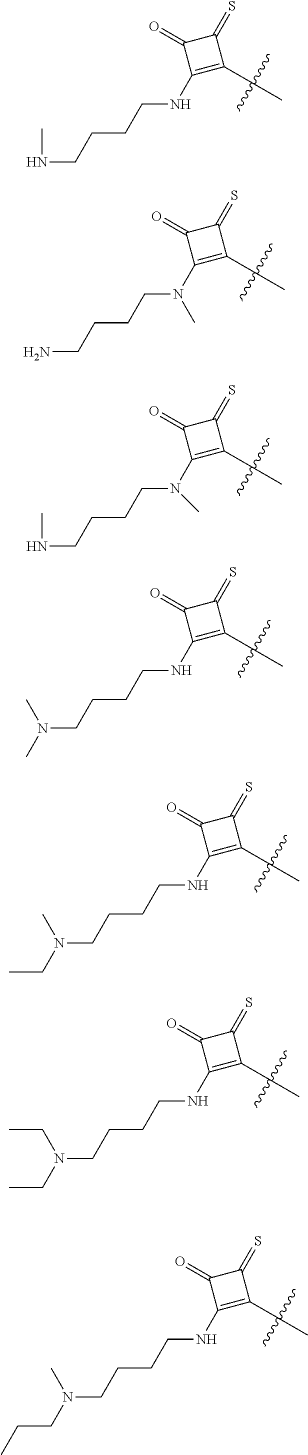

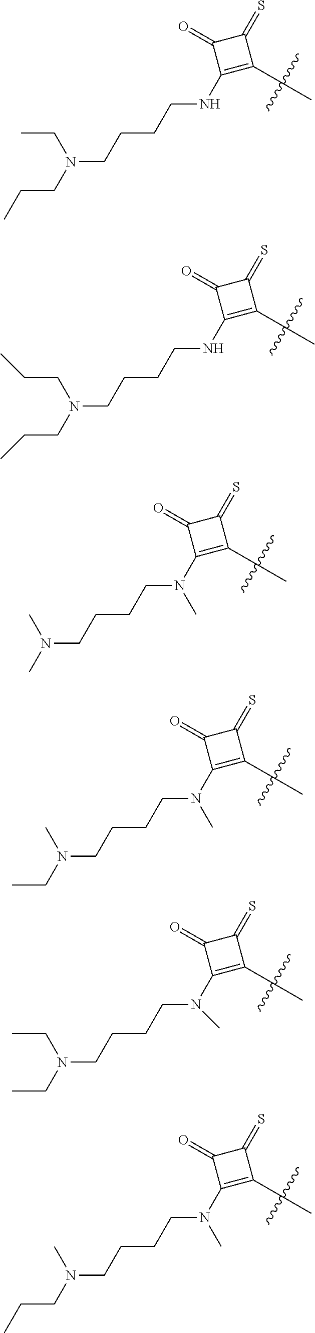 [SMILES: *c1c(NCCCCN(CC)CCC)c(=O)c1=S ; *c1c(NCCCCN(CCC)CCC)c(=O)c1=S ; *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S]

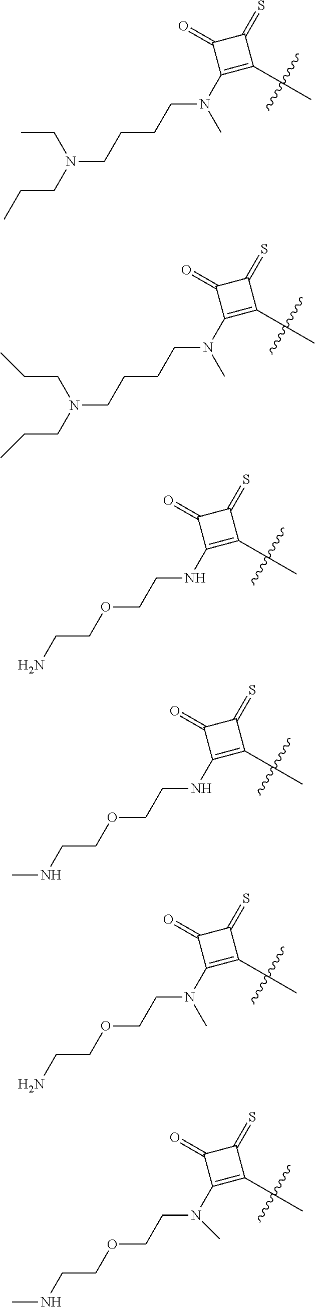 [SMILES: *c1c(N(C)CCCCN(CC)CCC)c(=O)c1=S ; *c1c(N(C)CCCCN(CCC)CCC)c(=O)c1=S ; *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S]

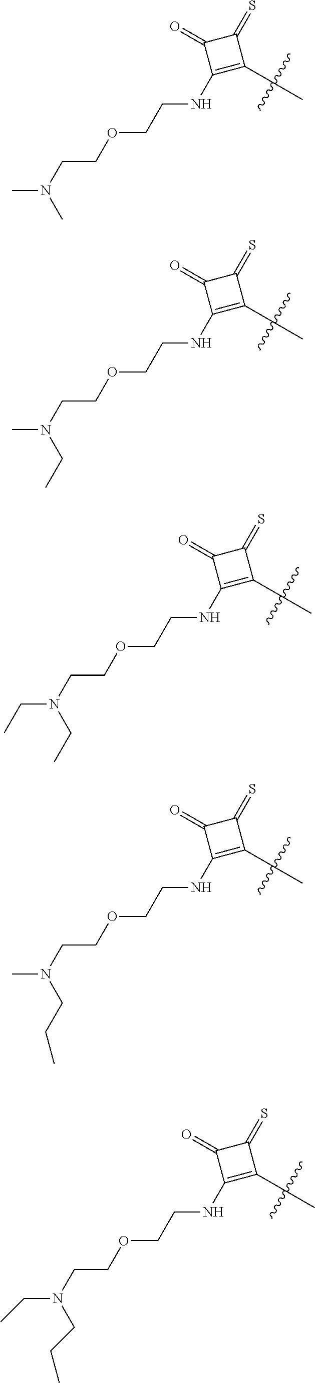 [SMILES: *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S ; *c1c(NCCOCCN(CC)CCC)c(=O)c1=S]

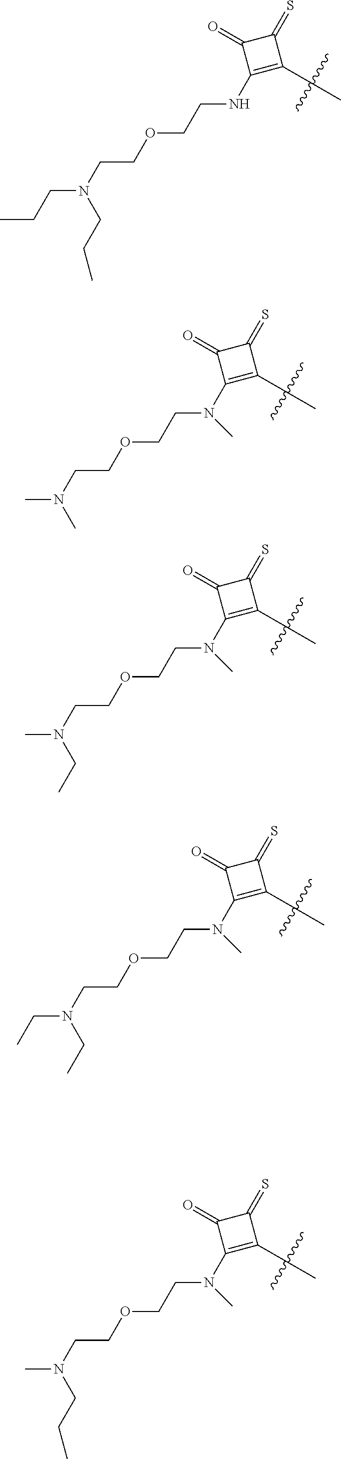 [SMILES: *c1c(NCCOCCN(CCC)CCC)c(=O)c1=S ; *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S]

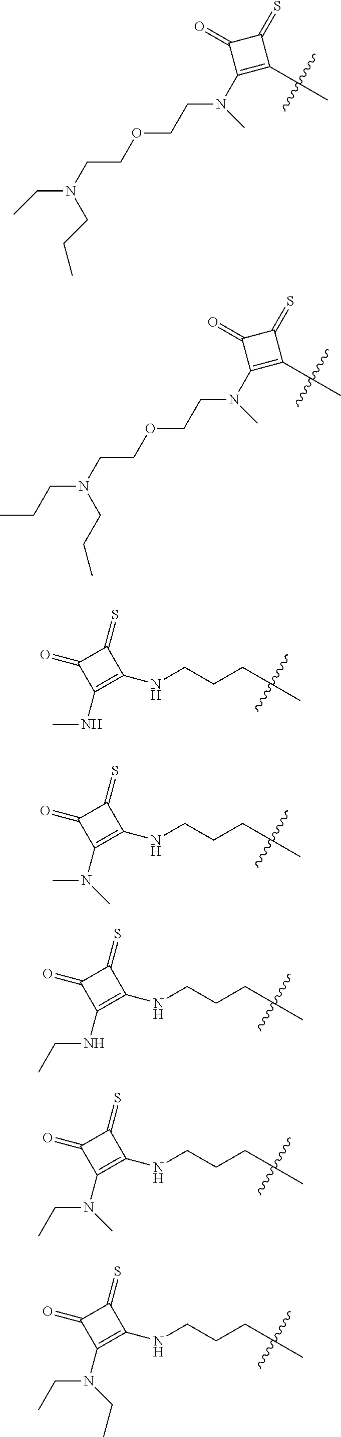

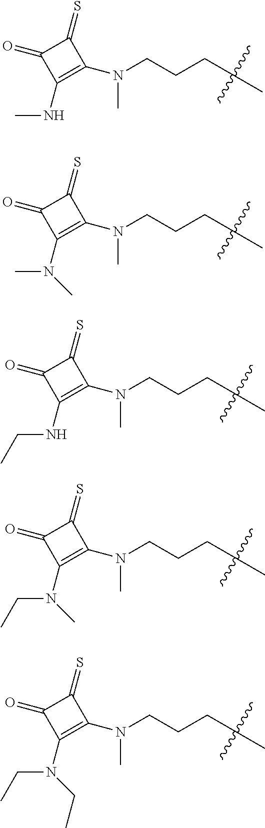

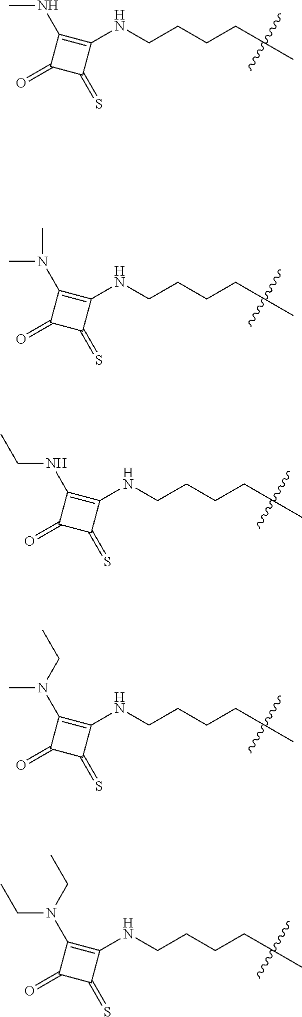 [SMILES: *CCCCNc1c(NC)c(=O)c1=S ; *CCCCNc1c(N(C)C)c(=O)c1=S ; *CCCCNc1c(NCC)c(=O)c1=S ; *CCCCNc1c(N(C)CC)c(=O)c1=S ; *CCCCNc1c(N(CC)CC)c(=O)c1=S]

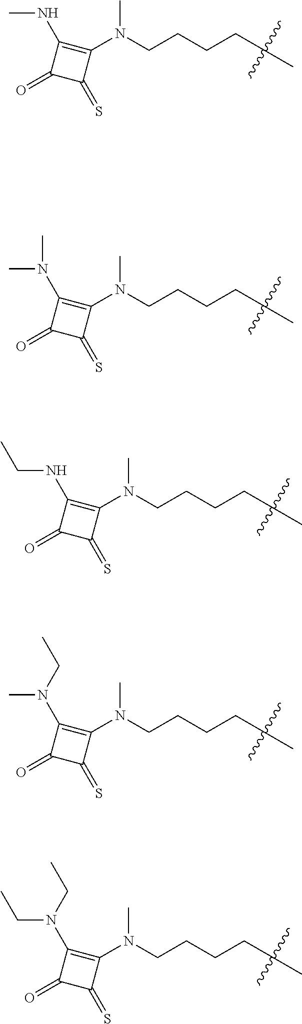 [SMILES: *CCCCN(C)c1c(NC)c(=O)c1=S ; *CCCCN(C)c1c(N(C)C)c(=O)c1=S ; *CCCCN(C)c1c(NCC)c(=O)c1=S ; *CCCCN(C)c1c(N(C)CC)c(=O)c1=S ; *CCCCN(C)c1c(N(CC)CC)c(=O)c1=S]

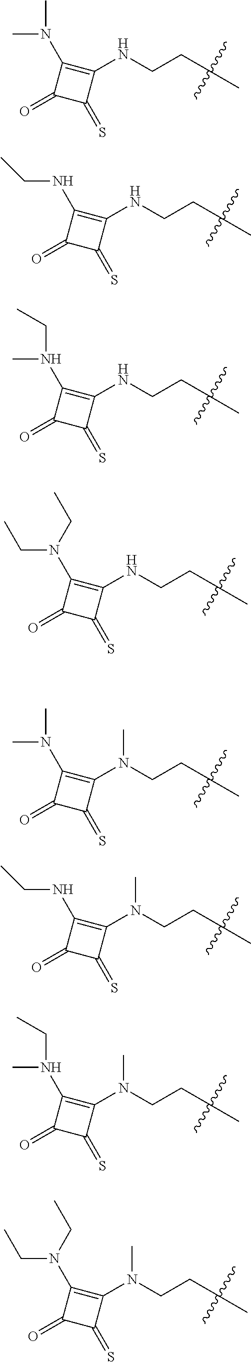

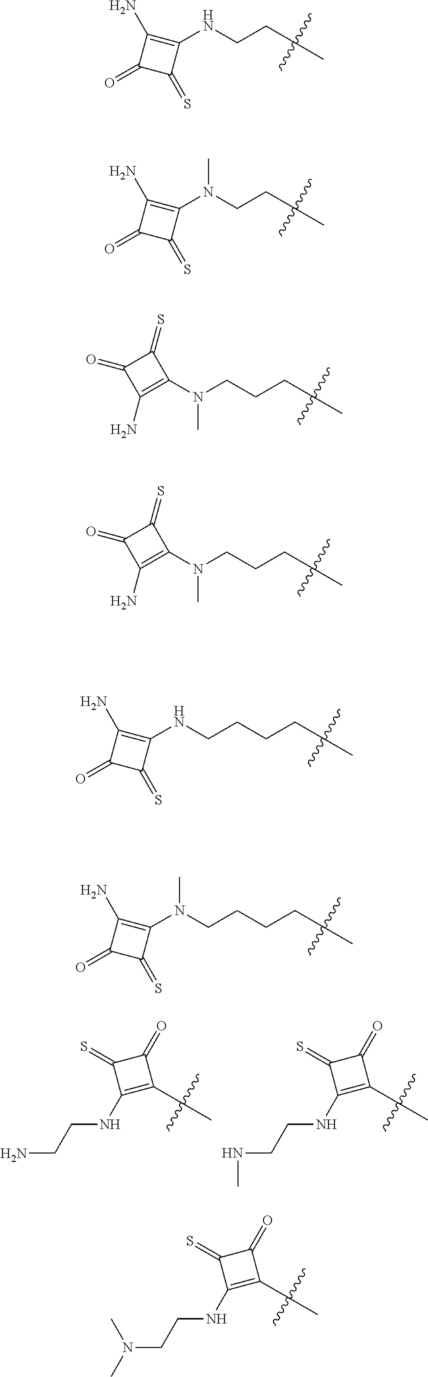

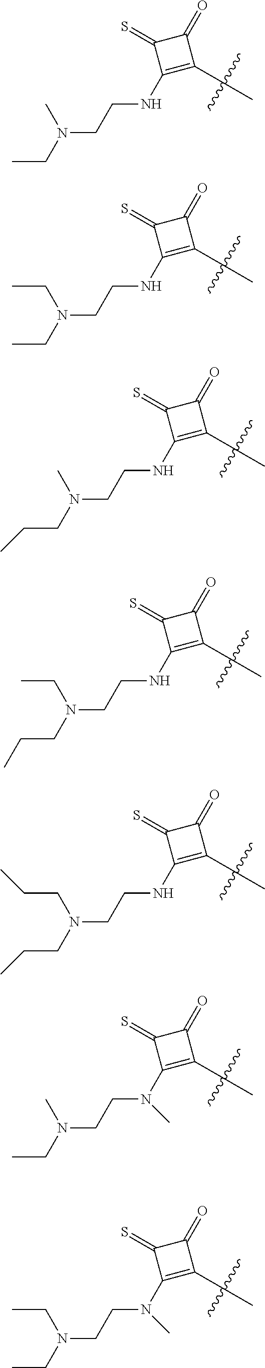

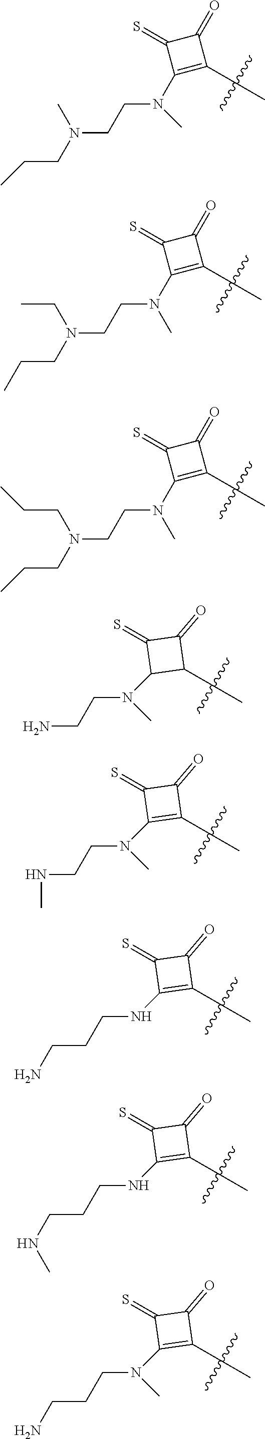

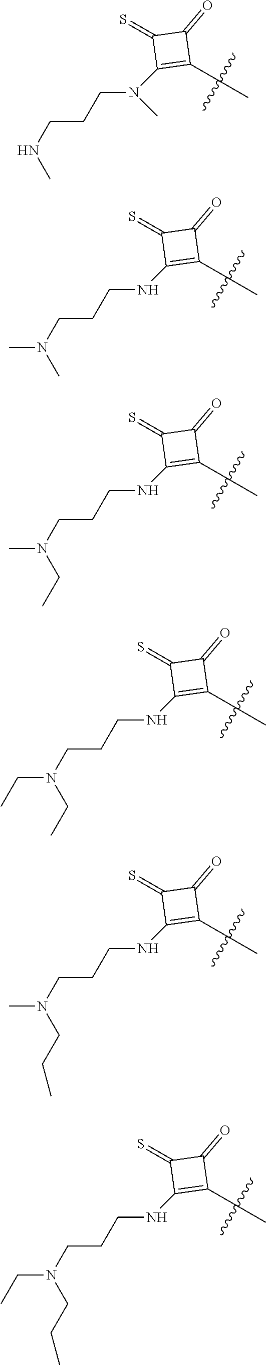 [SMILES: *c1c(N(C)CCCNC)c(=S)c1=O ; *c1c(NCCCN(C)C)c(=S)c1=O ; *c1c(NCCCN(C)CC)c(=S)c1=O ; *c1c(NCCCN(CC)CC)c(=S)c1=O ; *c1c(NCCCN(C)CCC)c(=S)c1=O ; *c1c(NCCCN(CC)CCC)c(=S)c1=O]

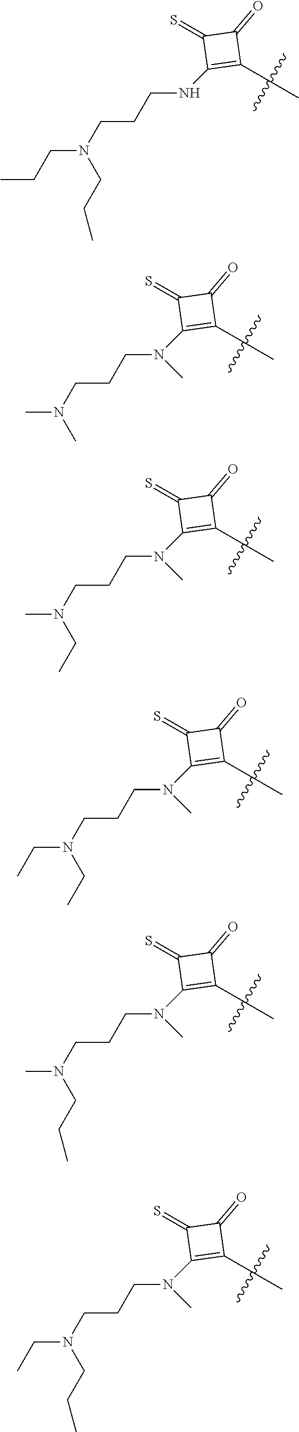 [SMILES: *c1c(NCCCN(CCC)CCC)c(=S)c1=O ; *c1c(N(C)CCCN(C)C)c(=S)c1=O ; *c1c(N(C)CCCN(C)CC)c(=S)c1=O ; *c1c(N(C)CCCN(CC)CC)c(=S)c1=O ; *c1c(N(C)CCCN(C)CCC)c(=S)c1=O ; *c1c(N(C)CCCN(CC)CCC)c(=S)c1=O]

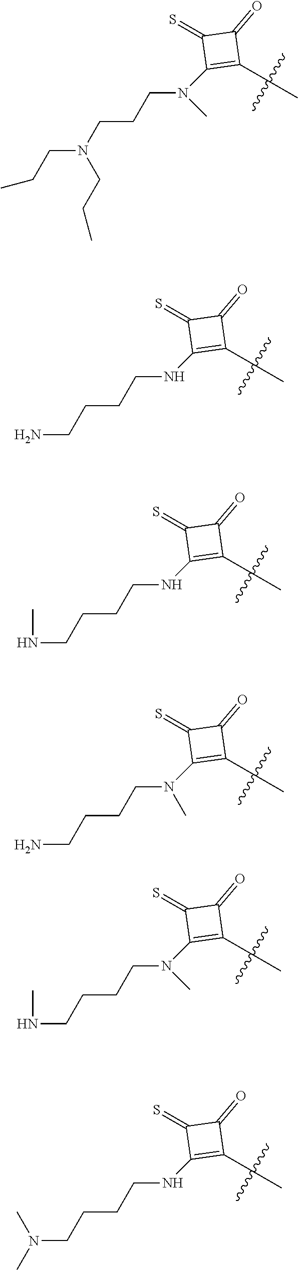 [SMILES: *c1c(N(C)CCCN(CCC)CCC)c(=S)c1=O ; *c1c(NCCCCN)c(=S)c1=O ; *c1c(NCCCCNC)c(=S)c1=O ; *c1c(N(C)CCCCN)c(=S)c1=O ; *c1c(N(C)CCCCNC)c(=S)c1=O ; *c1c(NCCCCN(C)C)c(=S)c1=O]

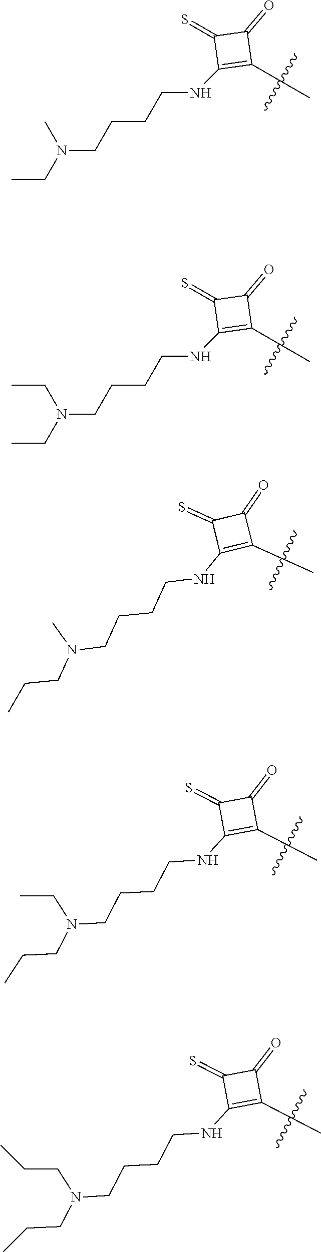 [SMILES: *c1c(NCCCCN(C)CC)c(=S)c1=O ; *c1c(NCCCCN(CC)CC)c(=S)c1=O ; *c1c(NCCCCN(C)CCC)c(=S)c1=O ; *c1c(NCCCCN(CC)CCC)c(=S)c1=O ; *c1c(NCCCCN(CCC)CCC)c(=S)c1=O]

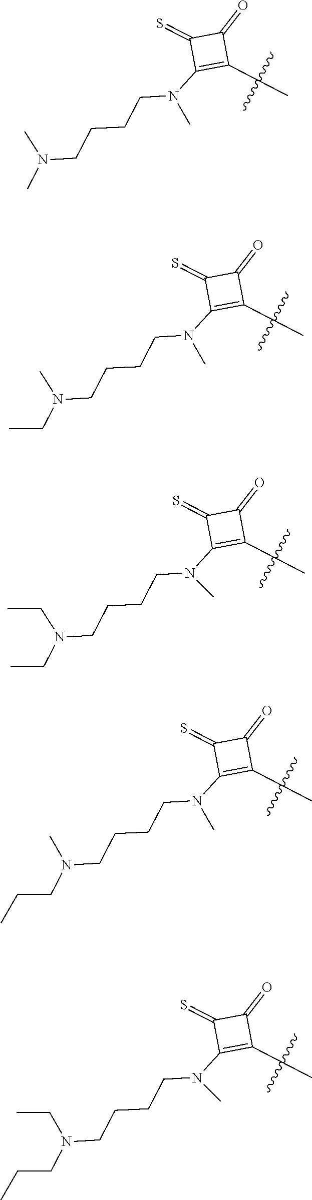 [SMILES: *c1c(N(C)CCCCN(C)C)c(=S)c1=O ; *c1c(N(C)CCCCN(C)CC)c(=S)c1=O ; *c1c(N(C)CCCCN(CC)CC)c(=S)c1=O ; *c1c(N(C)CCCCN(C)CCC)c(=S)c1=O ; *c1c(N(C)CCCCN(CC)CCC)c(=S)c1=O]

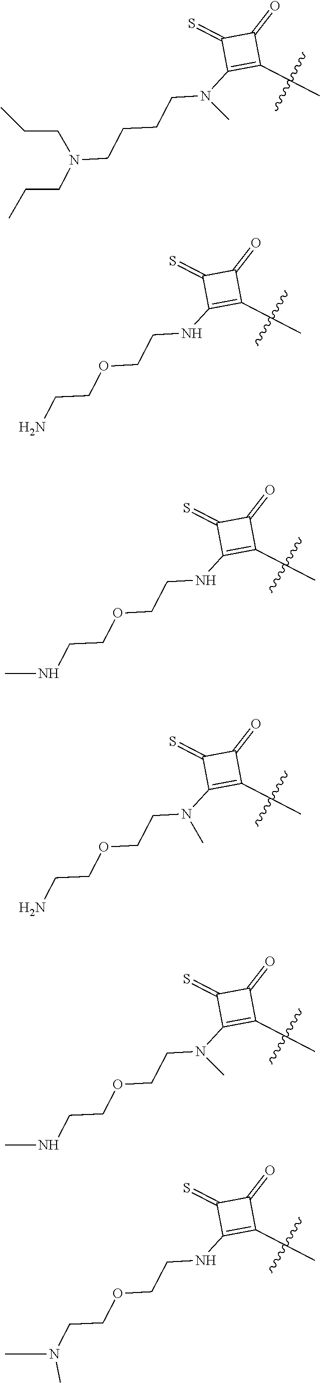 [SMILES: *c1c(N(C)CCCCN(CCC)CCC)c(=S)c1=O ; *c1c(NCCOCCN)c(=S)c1=O ; *c1c(NCCOCCNC)c(=S)c1=O ; *c1c(N(C)CCOCCN)c(=S)c1=O ; *c1c(N(C)CCOCCNC)c(=S)c1=O ; *c1c(NCCOCCN(C)C)c(=S)c1=O]

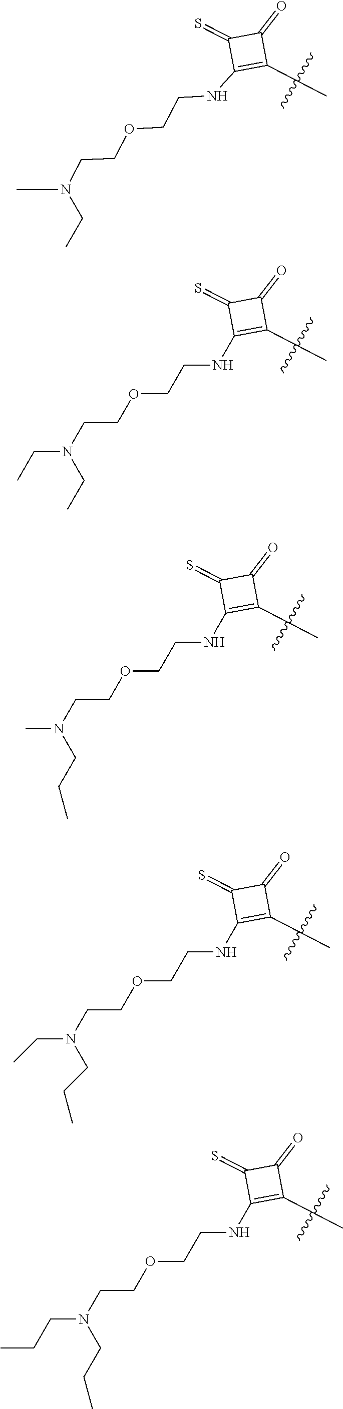 [SMILES: *c1c(NCCOCCN(C)CC)c(=S)c1=O ; *c1c(NCCOCCN(CC)CC)c(=S)c1=O ; *c1c(NCCOCCN(C)CCC)c(=S)c1=O ; *c1c(NCCOCCN(CC)CCC)c(=S)c1=O ; *c1c(NCCOCCN(CCC)CCC)c(=S)c1=O]

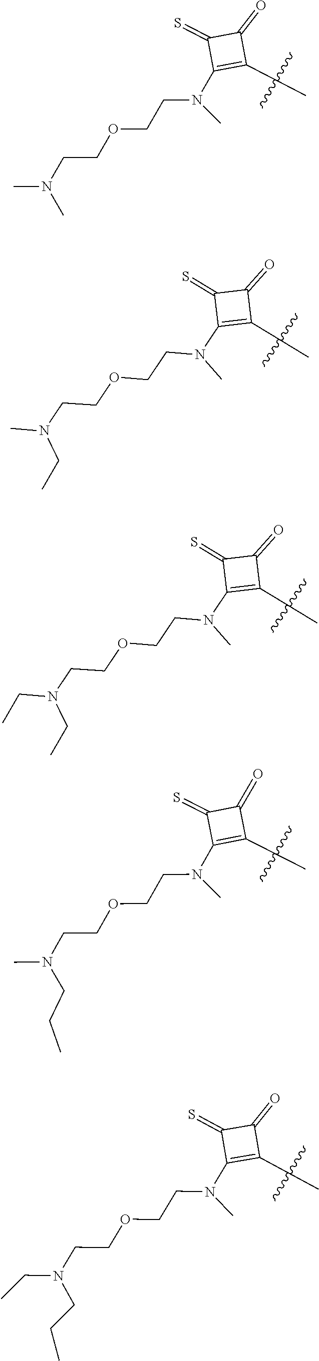 [SMILES: *c1c(N(C)CCOCCN(C)C)c(=S)c1=O ; *c1c(N(C)CCOCCN(C)CC)c(=S)c1=O ; *c1c(N(C)CCOCCN(CC)CC)c(=S)c1=O ; *c1c(N(C)CCOCCN(C)CCC)c(=S)c1=O ; *c1c(N(C)CCOCCN(CC)CCC)c(=S)c1=O]

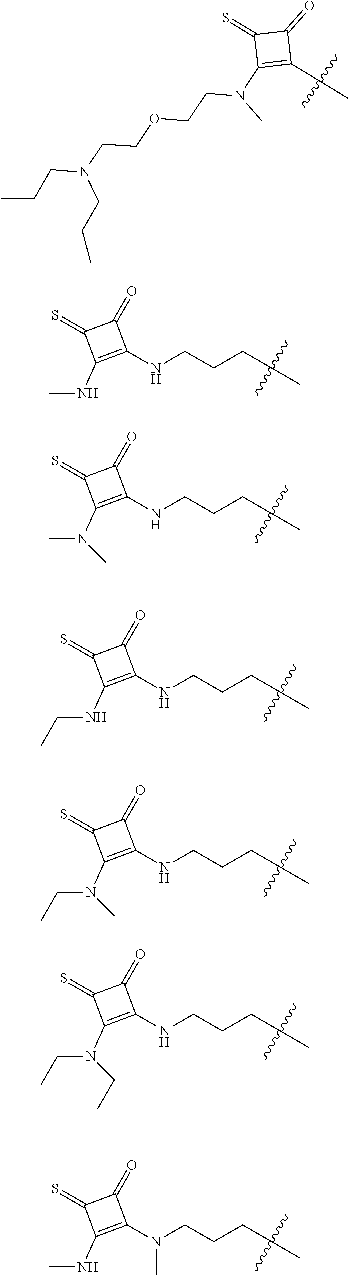

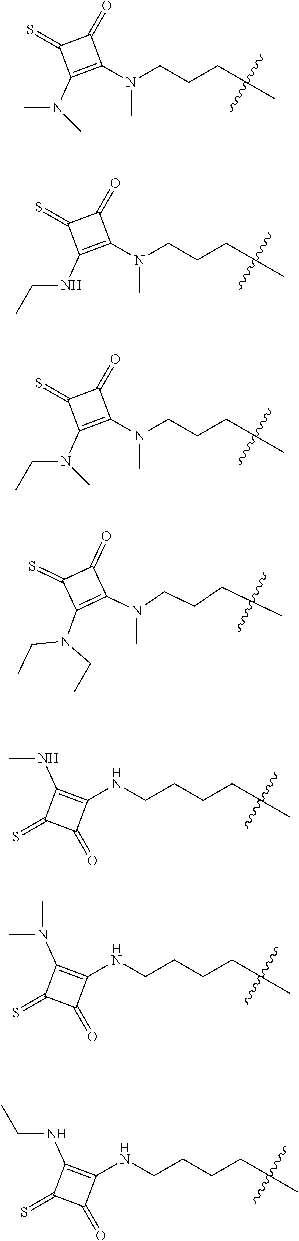

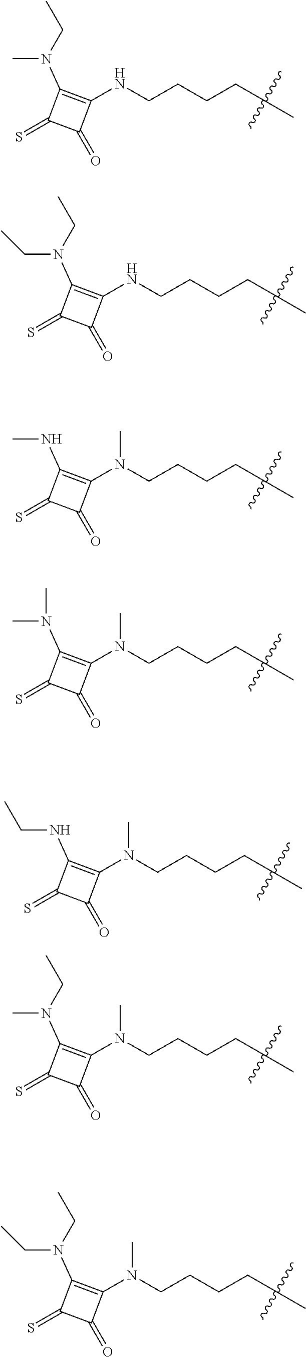

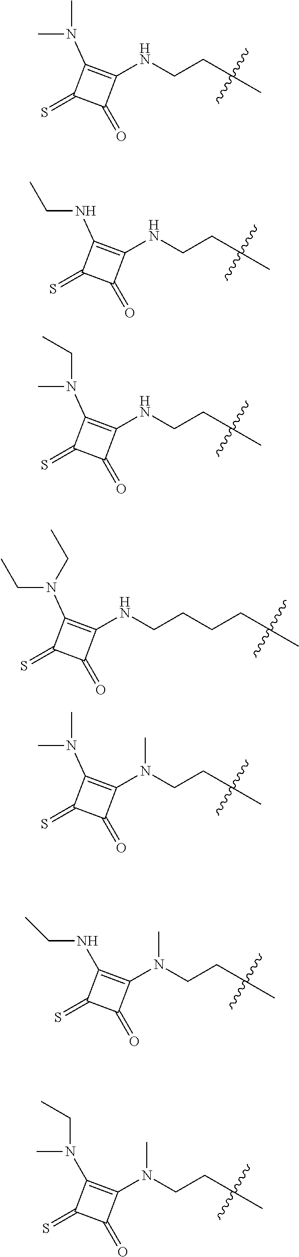

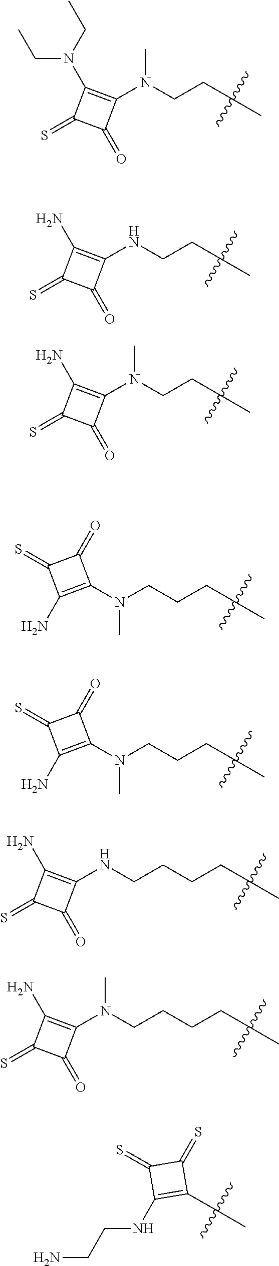

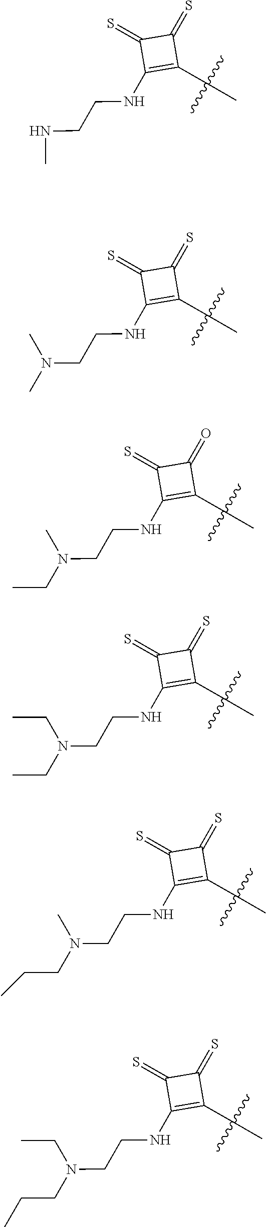 [SMILES: *c1c(NCCNC)c(=S)c1=S ; *c1c(NCCN(C)C)c(=S)c1=S ; *c1c(NCCN(C)CC)c(=S)c1=O ; *c1c(NCCN(CC)CC)c(=S)c1=S ; *c1c(NCCN(C)CCC)c(=S)c1=S ; *c1c(NCCN(CC)CCC)c(=S)c1=S]

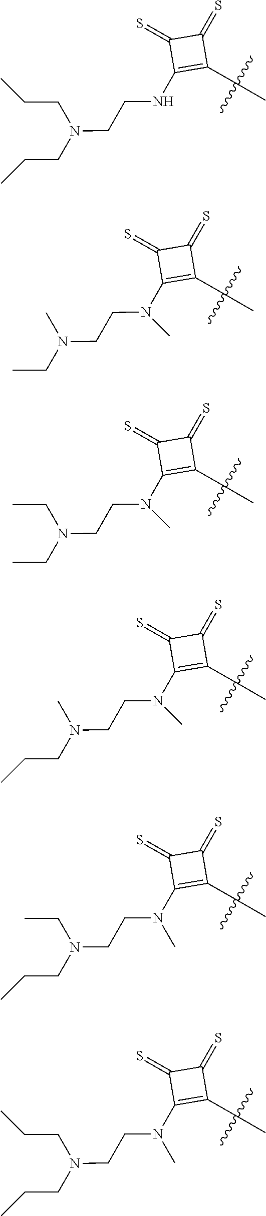 [SMILES: *c1c(NCCN(CCC)CCC)c(=S)c1=S ; *c1c(N(C)CCN(C)CC)c(=S)c1=S ; *c1c(N(C)CCN(CC)CC)c(=S)c1=S ; *c1c(N(C)CCN(C)CCC)c(=S)c1=S ; *c1c(N(C)CCN(CC)CCC)c(=S)c1=S ; *c1c(N(C)CCN(CCC)CCC)c(=S)c1=S]

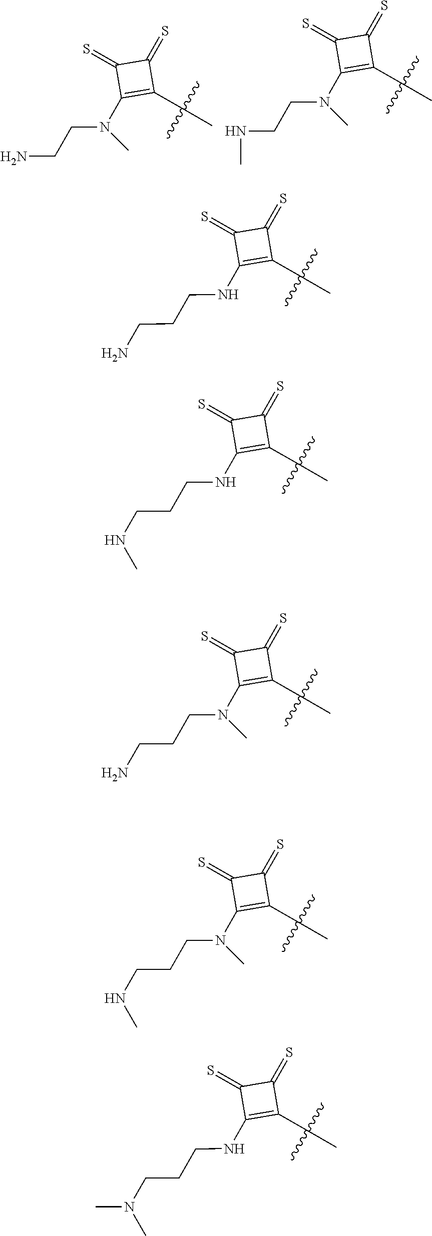

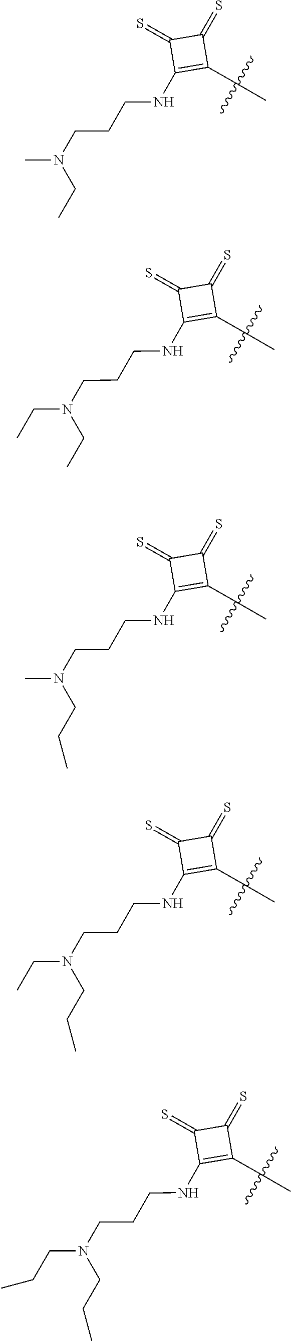 [SMILES: *c1c(NCCCN(C)CC)c(=S)c1=S ; *c1c(NCCCN(CC)CC)c(=S)c1=S ; *c1c(NCCCN(C)CCC)c(=S)c1=S ; *c1c(NCCCN(CC)CCC)c(=S)c1=S ; *c1c(NCCCN(CCC)CCC)c(=S)c1=S]

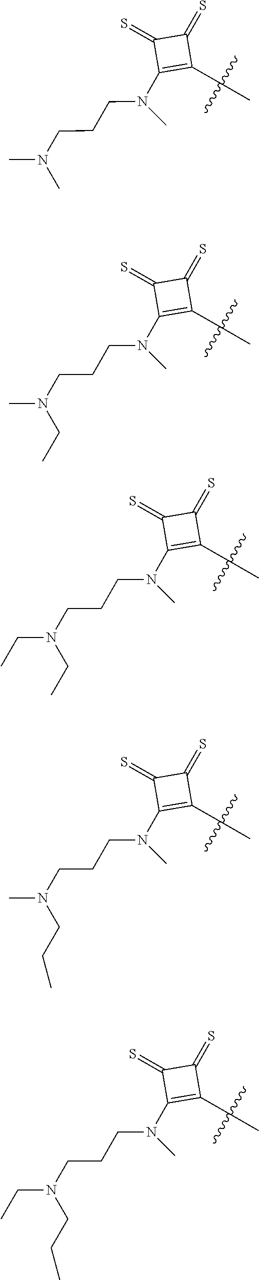 [SMILES: *c1c(N(C)CCCN(C)C)c(=S)c1=S ; *c1c(N(C)CCCN(C)CC)c(=S)c1=S ; *c1c(N(C)CCCN(CC)CC)c(=S)c1=S ; *c1c(N(C)CCCN(C)CCC)c(=S)c1=S ; *c1c(N(C)CCCN(CC)CCC)c(=S)c1=S]

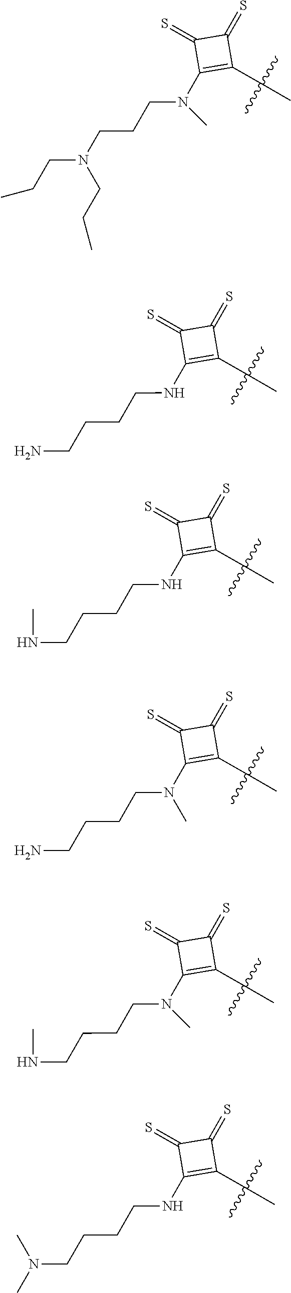 [SMILES: *c1c(N(C)CCCN(CCC)CCC)c(=S)c1=S ; *c1c(NCCCCN)c(=S)c1=S ; *c1c(NCCCCNC)c(=S)c1=S ; *c1c(N(C)CCCCN)c(=S)c1=S ; *c1c(N(C)CCCCNC)c(=S)c1=S ; *c1c(NCCCCN(C)C)c(=S)c1=S]

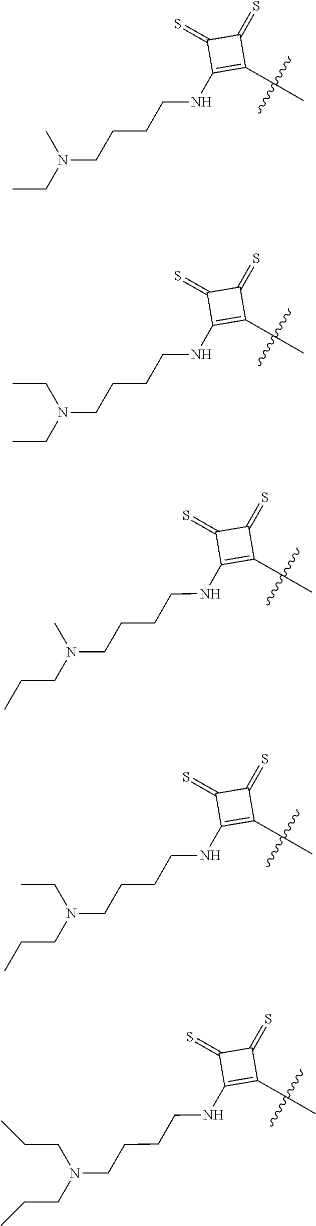 [SMILES: *c1c(NCCCCN(C)CC)c(=S)c1=S ; *c1c(NCCCCN(CC)CC)c(=S)c1=S ; *c1c(NCCCCN(C)CCC)c(=S)c1=S ; *c1c(NCCCCN(CC)CCC)c(=S)c1=S ; *c1c(NCCCCN(CCC)CCC)c(=S)c1=S]

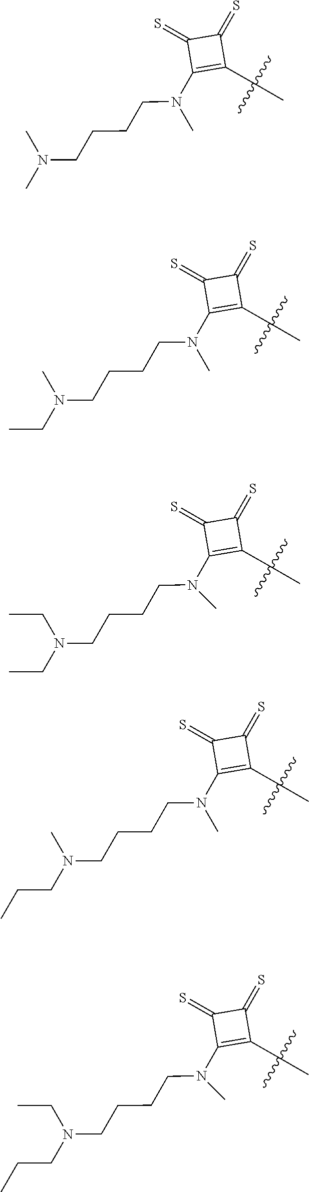 [SMILES: *c1c(N(C)CCCCN(C)C)c(=S)c1=S ; *c1c(N(C)CCCCN(C)CC)c(=S)c1=S ; *c1c(N(C)CCCCN(CC)CC)c(=S)c1=S ; *c1c(N(C)CCCCN(C)CCC)c(=S)c1=S ; *c1c(N(C)CCCCN(CC)CCC)c(=S)c1=S]

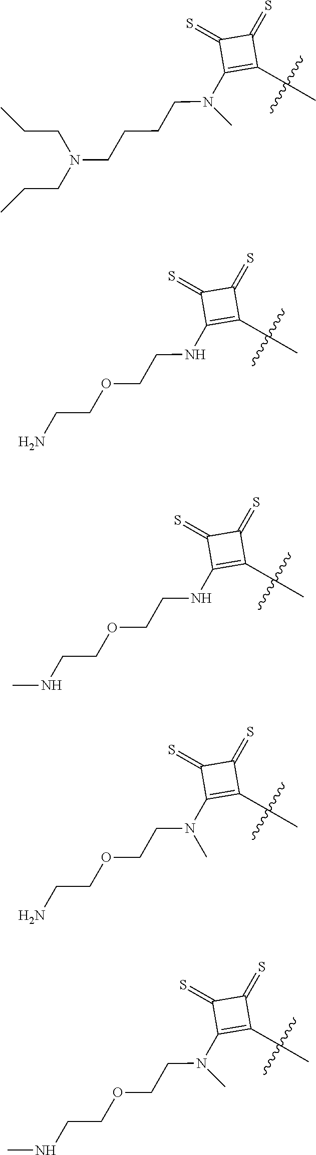 [SMILES: *c1c(N(C)CCCCN(CCC)CCC)c(=S)c1=S ; *c1c(NCCOCCN)c(=S)c1=S ; *c1c(NCCOCCNC)c(=S)c1=S ; *c1c(N(C)CCOCCN)c(=S)c1=S ; *c1c(N(C)CCOCCNC)c(=S)c1=S]

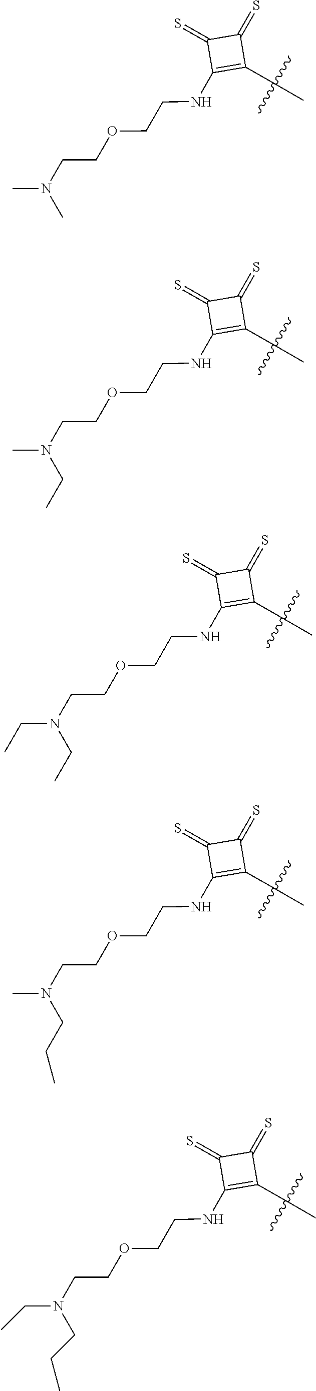 [SMILES: *c1c(NCCOCCN(C)C)c(=S)c1=S ; *c1c(NCCOCCN(C)CC)c(=S)c1=S ; *c1c(NCCOCCN(CC)CC)c(=S)c1=S ; *c1c(NCCOCCN(C)CCC)c(=S)c1=S ; *c1c(NCCOCCN(CC)CCC)c(=S)c1=S]

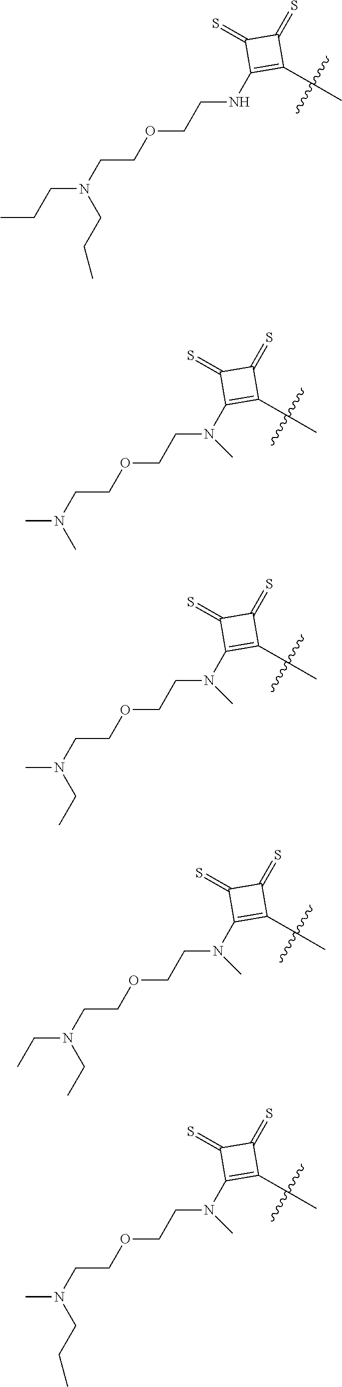 [SMILES: *c1c(NCCOCCN(CCC)CCC)c(=S)c1=S ; *c1c(N(C)CCOCCN(C)C)c(=S)c1=S ; *c1c(N(C)CCOCCN(C)CC)c(=S)c1=S ; *c1c(N(C)CCOCCN(CC)CC)c(=S)c1=S ; *c1c(N(C)CCOCCN(C)CCC)c(=S)c1=S]

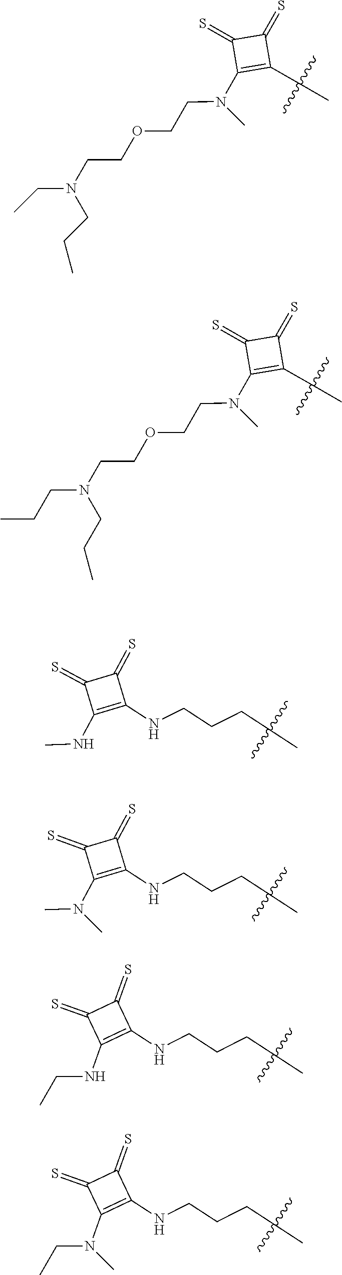

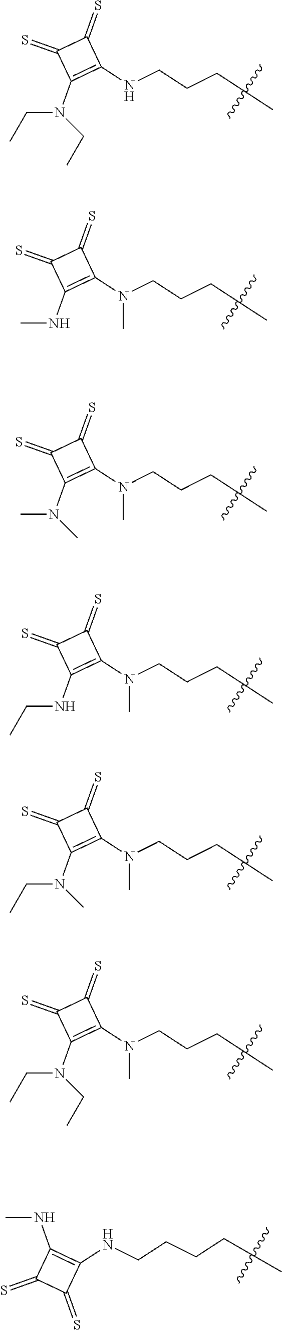

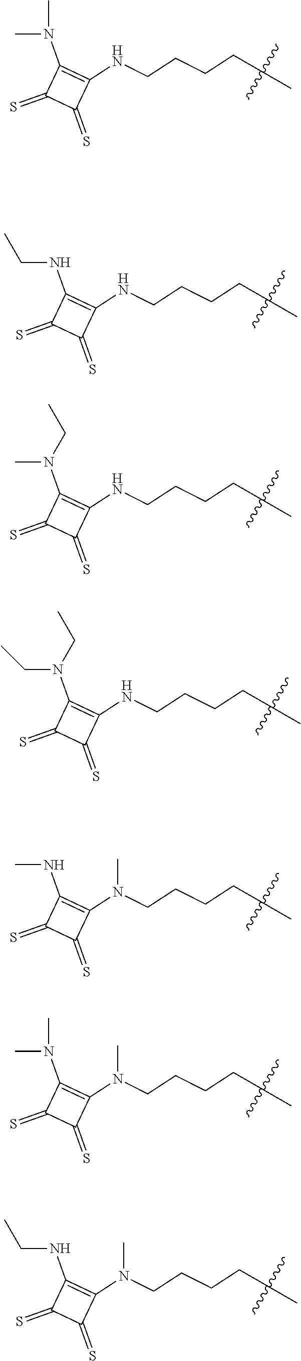

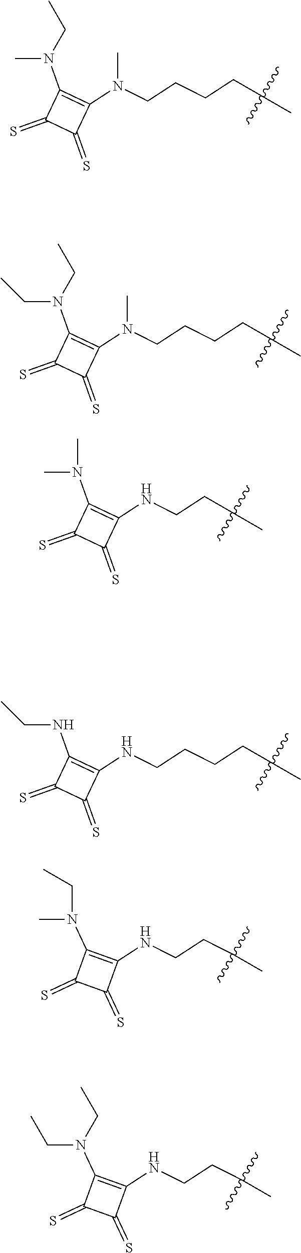

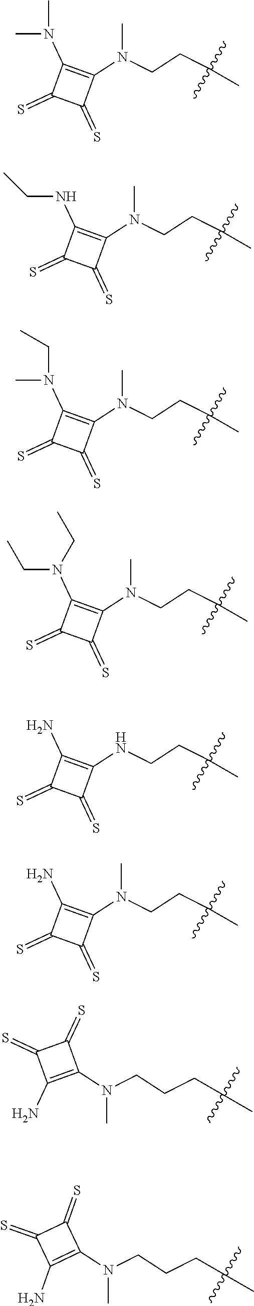

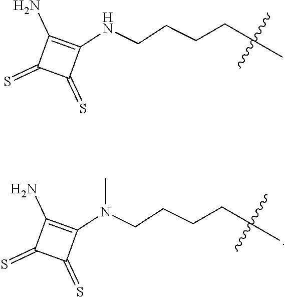 [SMILES: *CCCCNc1c(N)c(=S)c1=S ; *CCCCN(C)c1c(N)c(=S)c1=S]

In some embodiments, R⁴ is selected from any of the following groups:

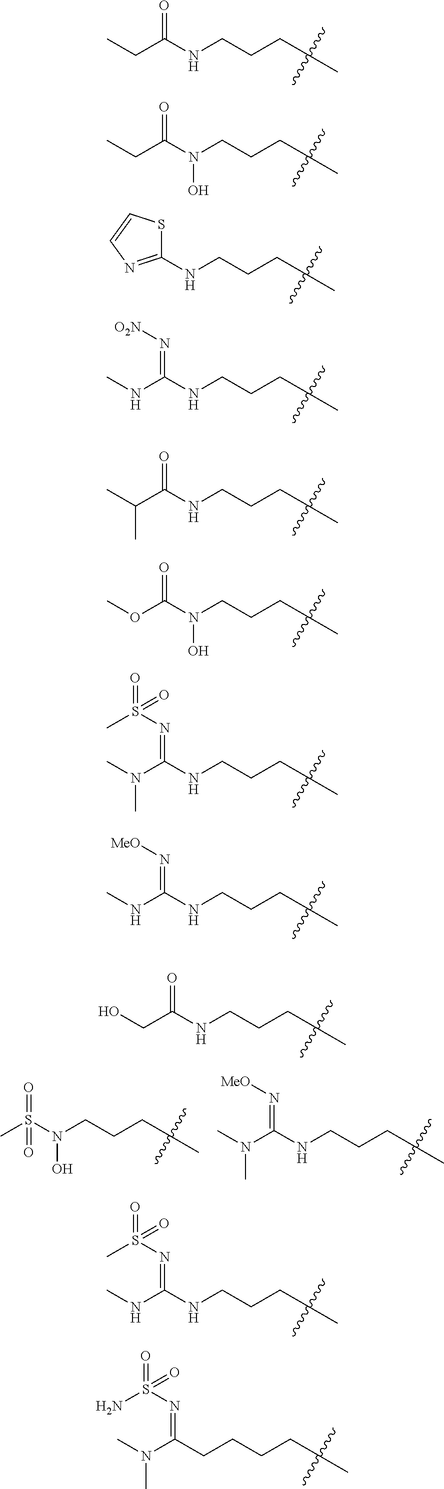

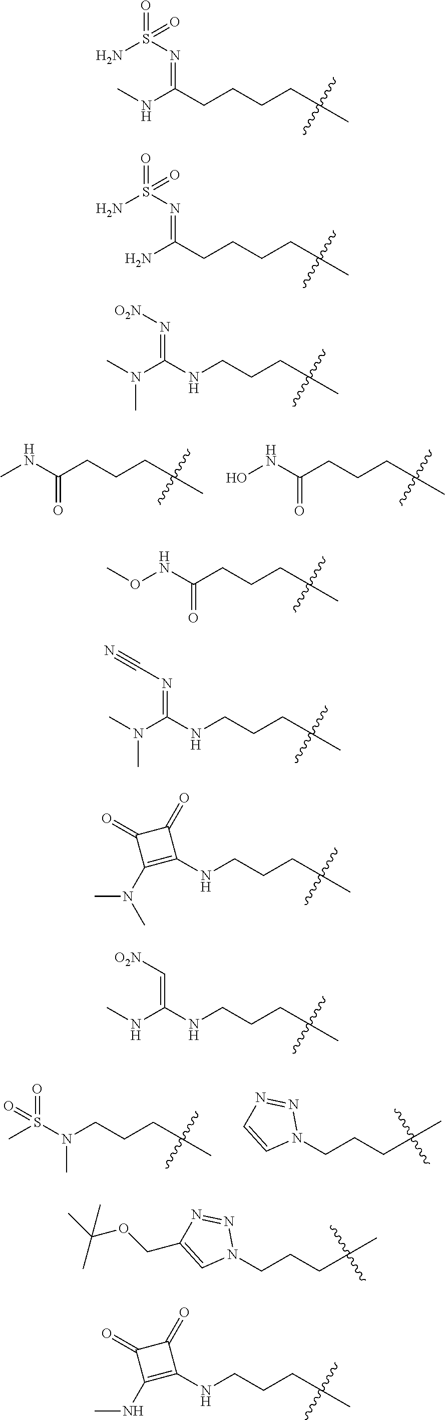

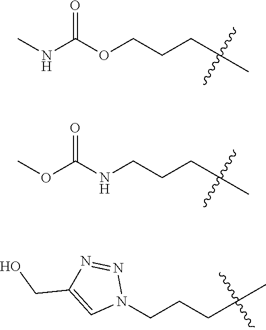

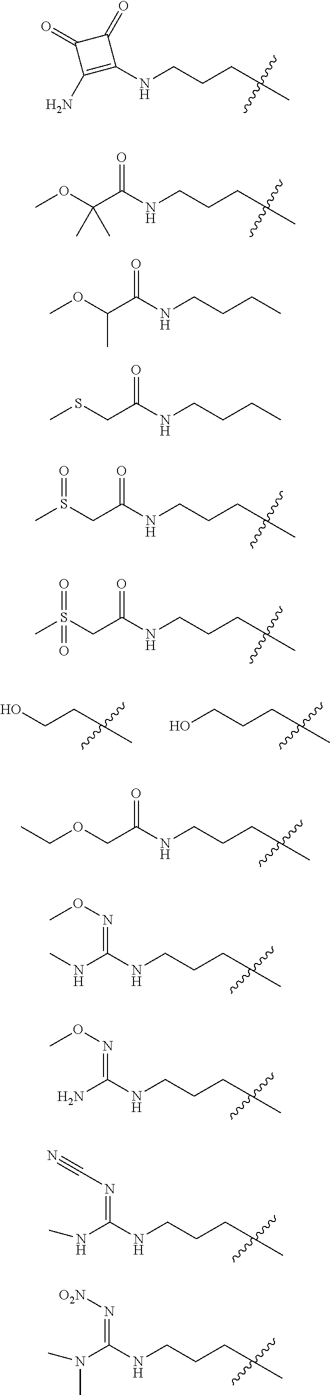

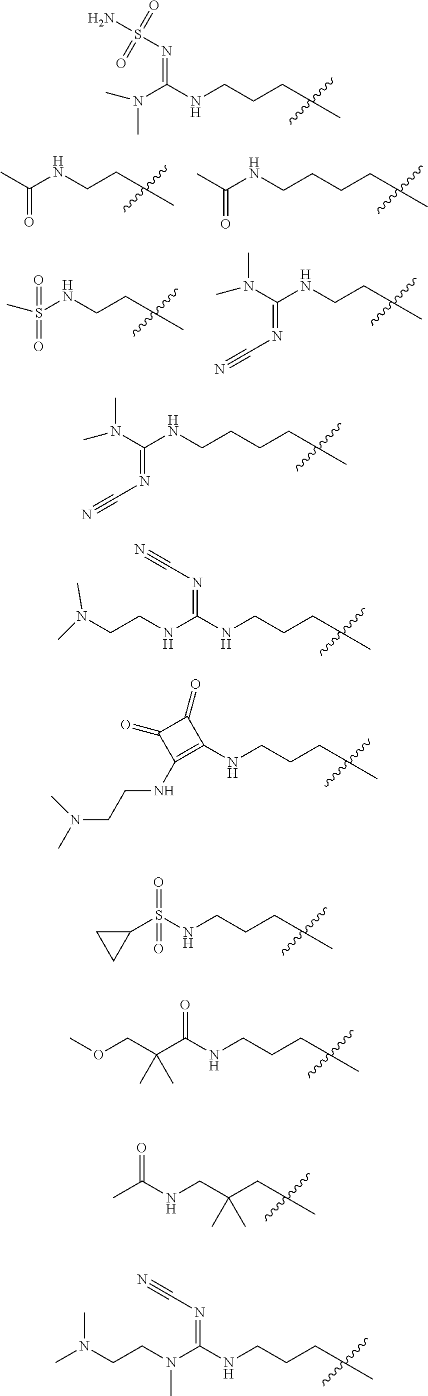

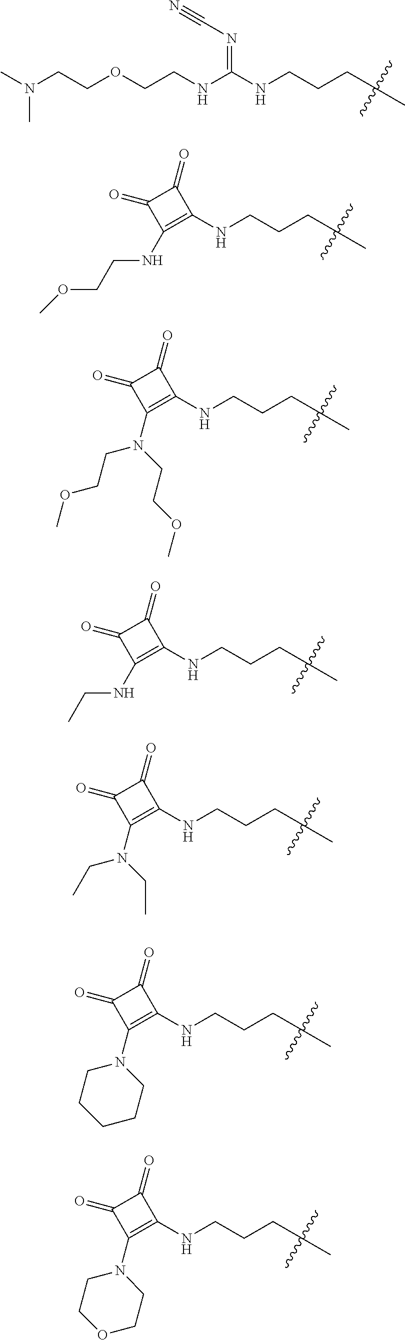

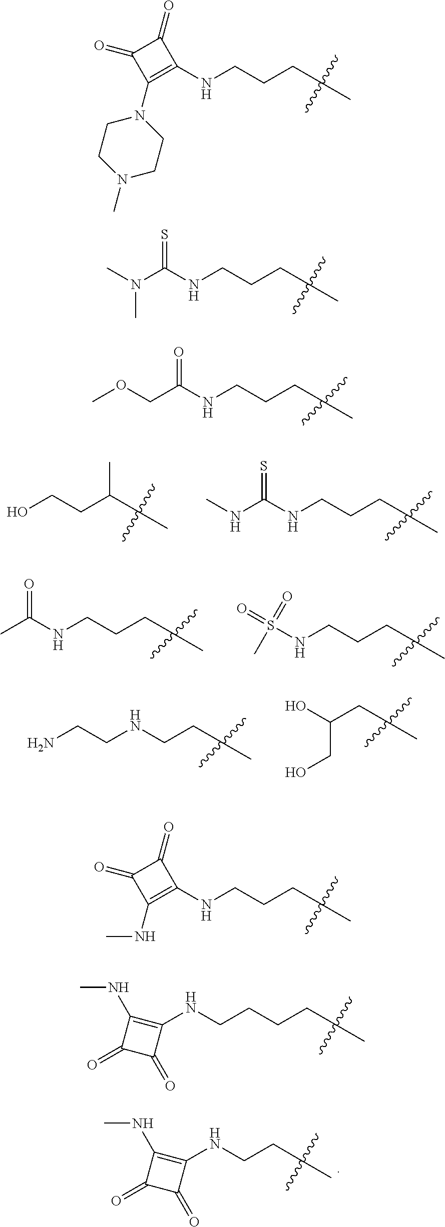

In some embodiments,

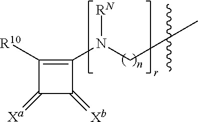

is selected from any of the following groups:

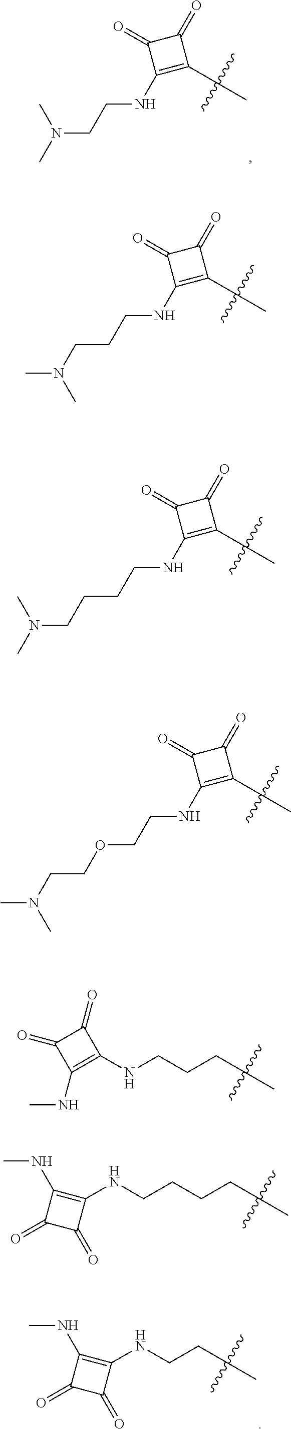

In some embodiments, a compound of Formula (III) further comprises an anion. As described herein, and anion can be any anion capable of reacting with an amine to form an ammonium salt. Examples include, hut are not limited to, chloride, bromide, iodide, fluoride, acetate, formate, trifluoroacetate, difluoroacetate, trichloroacetate, and phosphate.

In some embodiments the compound of any of the formulae described herein is suitable for making a nanoparticle composition for intramuscular administration.

In some embodiments, R² and R³, together with the atom to which they are attached, form a heterocycle or carbocycle. In some embodiments, R² and R³, together with the atom to which they are attached, form a 5- to 14-membered aromatic or non-aromatic heterocycle having one or more heteroatoms selected from N, O, S, and P. In some embodiments, R² and R³, together with the atom to which they are attached, form an optionally substituted C₃₋₂₀ carbocycle (e.g., C₃₋₁₈ carbocycle, C₃₋₁₅ carbocycle, C₃₋₁₂ carbocycle, or C₃₋₁₀ carbocycle), either aromatic or non-aromatic. In some embodiments, R² and R³, together with the atom to which they are attached, form a C₃₋₆ carbocycle. In other embodiments, R² and R³, together with the atom to which they are attached, form a C₆ carbocycle, such as a cyclohexyl or phenyl group. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a cyclohexyl or phenyl group bearing one or more C₅ alkyl substitutions. In certain embodiments, the heterocycle or C₃₋₆ carbocycle formed by R² and R³, is substituted with a carbocycle groups. For example, R² and R³, together with the atom to which they are attached, may form a cyclohexyl or phenyl group that is substituted with cyclohexyl. In some embodiments, R² and R³, together with the atom to which they are attached, form a C₇₋₁₅ carbocycle, such as a cycloheptyl, cyclopentadecanyl, or naphthyl group.

In some embodiments, R⁴ is selected from —(CH₂)ₙQ and —(CH₂)ₙCHQR. In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H) C(O)N(R)₂, —N(R)S(O)₂R⁸, —N(H)C(O)N(H)(R), —N(R)C(S)N(R)₂, —N(H)C(S)N(R)₂, —N(H)C(S)N(H)(R), and a heterocycle. In other embodiments, Q is selected from the group consisting of an imidazole, a pyrimidine, and a purine.

In some embodiments, R² and R³, together with the atom to which they are attached, form a heterocycle or carbocycle. In some embodiments, R² and R³, together with the atom to which they are attached, form a C₃₋₆ carbocycle. In some embodiments, R² and R³, together with the atom to which they are attached, form a C₆ carbocycle. In some embodiments, R² and R³, together with the atom to which they are attached, form a phenyl group. In some embodiments, R² and R³, together with the atom to which they are attached, form a cyclohexyl group. In some embodiments, R² and R³, together with the atom to which they are attached, form a heterocycle. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a phenyl group bearing one or more C₅ alkyl substitutions.

In some embodiments, at least one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at most one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at least one occurrence of R⁵ and R⁶ is methyl.

The compounds of any one of formula (VI), (VI-a), (VII), (VIIa), (VIIb), (VIIc), (VIId), (VIII), (VIIIa), (VIIIb), (VIIIc) or (VIIId) include one or more of the following features when applicable.

In some embodiments, r is 0. In some embodiments, r is 1.

In some embodiments, n is 2, 3, or 4. In some embodiments, n is 2. In some embodiments, n is 4. In some embodiments, n is not 3.

In some embodiments, R^{N} is H. In some embodiments, R^{N} is C₁₋₃ alkyl. For example, in some embodiments R^{N} is C₁ alkyl. For example, in some embodiments R^{N} is C₂ alkyl. For example, in some embodiments R^{N} is C₂ alkyl.

In some embodiments, X^{a} is O. In some embodiments, X^{a} is S. In some embodiments, X^{b} is O. In some embodiments, X⁶ is S.

In some embodiments, R¹⁰ is selected from the group consisting of N(R)₂, —NH(CH₂)ₜ₁N(R)₂, —NH(CH₂)ₚ₁O(CH₂)_{q1}N(R)₂, —NH(CH₂)R, —N((CH₂)ₛ₁OR)₂, and a heterocycle.

In some embodiments, R¹⁰ is selected from the group consisting of —NH(CH₂)ₜ₁N(R)₂, —NH(CH₂)ₚ₁O(CH₂)_{q1}N(R)₂, —NH(CH₂)ₛ₁R, —N((CH₂)ₛ₁OR)₂, and a heterocycle.

In some embodiments wherein R¹⁰ is —NH(CH₂)ₒN(R)₂, o is 2, 3, or 4.

In some embodiments wherein —NH(CH₂)ₚ₁O(CH₂)_{q1}N(R)₂, p¹ is 2. In some embodiments wherein —NH(CH₂)ₚ₁O(CH₂)_{q1}N(R)₂, q¹ is 2.

In some embodiments wherein R¹⁰ is —N((CH₂)ₛ₁OR)₂, s¹ is 2.

In some embodiments wherein R¹⁰ is —NH(CH₂)ₒN(R)₂, —NH(CH₂)ₚO(CH₂)_{q}N(R)₂, —NH(CH₂)ₛOR, or —N((CH₂)ₛOR)₂, R is H or C₁-C₃ alkyl. For example, in some embodiments, R is C₁ alkyl. For example, in some embodiments, R is C₂ alkyl. For example, in some embodiments, R is H. For example, in some embodiments, R is H and one R is C₁-C₃ alkyl. For example, in some embodiments, R is H and one R is C₁ alkyl. For example, in some embodiments, R is H and one R is C₂ alkyl. In some embodiments wherein R¹⁰ is —NH(CH₂)ₜ₁N(R)₂, —NH(CH₂)ₚ₁O(CH₂)_{q1}N(R)₂, —NH(CH₂)ₛ₁OR, or —N((CH₂)ₛ₁OR)₂, each R is C₂-C₄ alkyl.

For example, in some embodiments, one R is H and one R is C₂-C₄ alkyl. In some embodiments, R¹⁰ is a heterocycle. For example, in some embodiments, R¹⁰ is morpholinyl. For example, in some embodiments, R¹⁰ is methyhlpiperazinyl.

In some embodiments, each occurrence of R⁵ and R⁶ is H.

In some embodiments, the compound of Formula (I) is selected from the group consisting of:

| Cpd | Structure |
|---|---|
| I 1 | 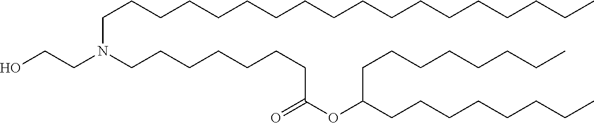 [SMILES: CCCCCCCCCCCCCCCCCCN(CCO)CCCCCCCC(=O)OC(CCCCCCCC)CCCCCCCC] |
| I 2 | 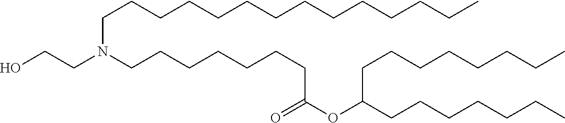 [SMILES: CCCCCCCCCCCCCCN(CCO)CCCCCCCC(=O)OC(CCCCCCC)CCCCCCCC] |
| I 3 | 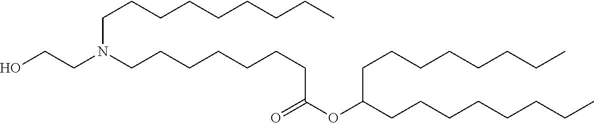 [SMILES: CCCCCCCCCN(CCO)CCCCCCCC(=O)OC(CCCCCCCC)CCCCCCCC] |
| I 4 | 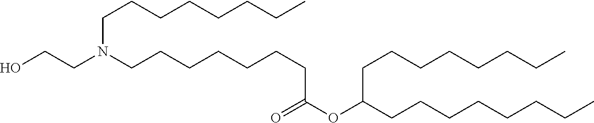 [SMILES: CCCCCCCCC(CCCCCCCC)OC(=O)CCCCCCCN(CCO)CCCCCCCC] |
| I 5 | 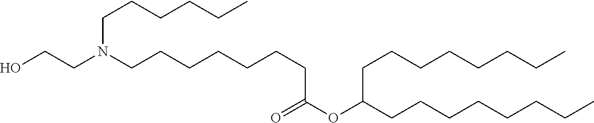 [SMILES: CCCCCCCCC(CCCCCCCC)OC(=O)CCCCCCCN(CCO)CCCCCC] |
| I 6 | 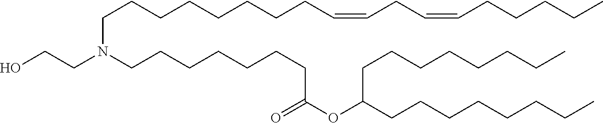 [SMILES: CCCCC/C=C\C/C=C\CCCCCCCCN(CCO)CCCCCCCC(=O)OC(CCCCCCCC)CCCCCCCC] |
| I 7 | 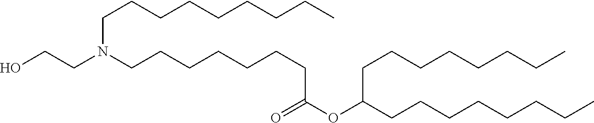 [SMILES: CCCCCCCCCN(CCO)CCCCCCCC(=O)OC(CCCCCCCC)CCCCCCCC] |
| I 8 | 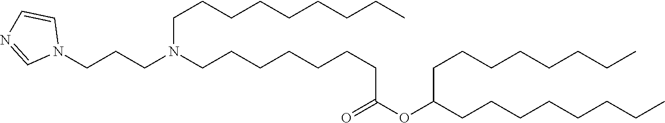 [SMILES: CCCCCCCCCN(CCCCCCCC(=O)OC(CCCCCCCC)CCCCCCCC)CCCn1ccnc1] |
| I 9 | 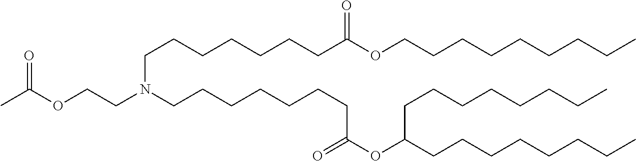 [SMILES: CCCCCCCCCOC(=O)CCCCCCCN(CCCCCCCC(=O)OC(CCCCCCCC)CCCCCCCC)CCOC(C)=O] |
| I 10 | 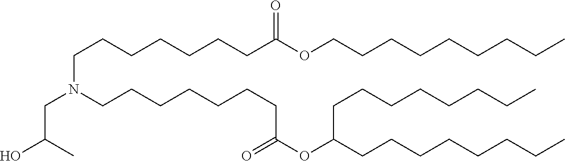 [SMILES: CCCCCCCCCOC(=O)CCCCCCCN(CCCCCCCC(=O)OC(CCCCCCCC)CCCCCCCC)CC(C)O] |
| I 11 | 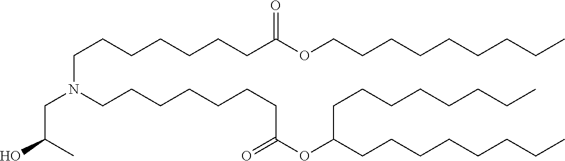 [SMILES: CCCCCCCCCOC(=O)CCCCCCCN(CCCCCCCC(=O)OC(CCCCCCCC)CCCCCCCC)C[C@@H](C)O] |
| I 12 | 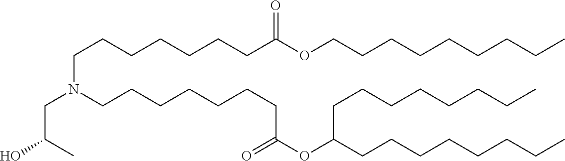 [SMILES: CCCCCCCCCOC(=O)CCCCCCCN(CCCCCCCC(=O)OC(CCCCCCCC)CCCCCCCC)C[C@H](C)O] |
| I 13 | 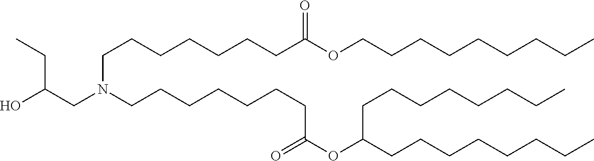 [SMILES: CCCCCCCCCOC(=O)CCCCCCCN(CCCCCCCC(=O)OC(CCCCCCCC)CCCCCCCC)CC(O)CC] |
| I 14 | 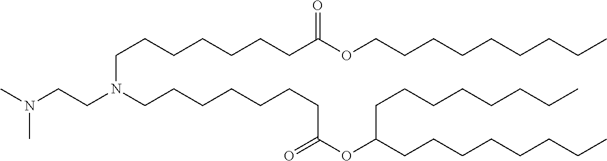 [SMILES: CCCCCCCCCOC(=O)CCCCCCCN(CCCCCCCC(=O)OC(CCCCCCCC)CCCCCCCC)CCN(C)C] |
| I 15 | 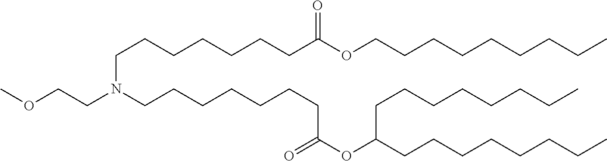 [SMILES: CCCCCCCCCOC(=O)CCCCCCCN(CCCCCCCC(=O)OC(CCCCCCCC)CCCCCCCC)CCOC] |
| I 16 | 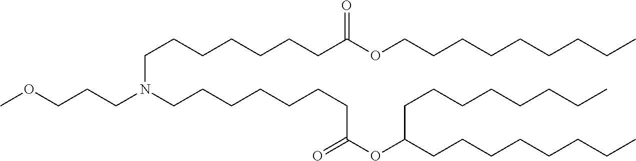 [SMILES: CCCCCCCCCOC(=O)CCCCCCCN(CCCCCCCC(=O)OC(CCCCCCCC)CCCCCCCC)CCCOC] |
| I 17 | 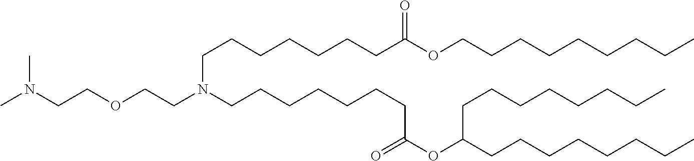 [SMILES: CCCCCCCCCOC(=O)CCCCCCCN(CCCCCCCC(=O)OC(CCCCCCCC)CCCCCCCC)CCOCCN(C)C] |
| I 18 | 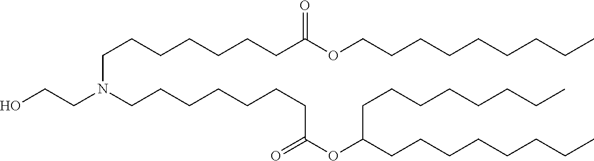 [SMILES: CCCCCCCCCOC(=O)CCCCCCCN(CCO)CCCCCCCC(=O)OC(CCCCCCCC)CCCCCCCC] |
| I 19 | 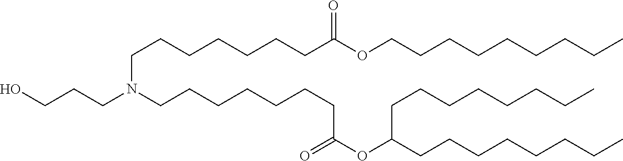 [SMILES: CCCCCCCCCOC(=O)CCCCCCCN(CCCO)CCCCCCCC(=O)OC(CCCCCCCC)CCCCCCCC] |
| I 20 | 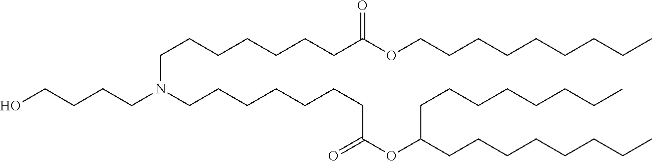 [SMILES: CCCCCCCCCOC(=O)CCCCCCCN(CCCCO)CCCCCCCC(=O)OC(CCCCCCCC)CCCCCCCC] |
| I 21 | 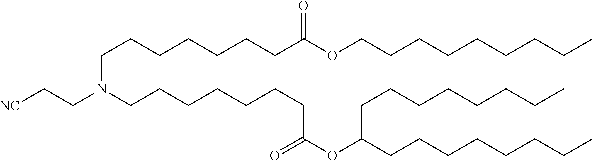 [SMILES: CCCCCCCCCOC(=O)CCCCCCCN(CCC#N)CCCCCCCC(=O)OC(CCCCCCCC)CCCCCCCC] |
| I 22 | 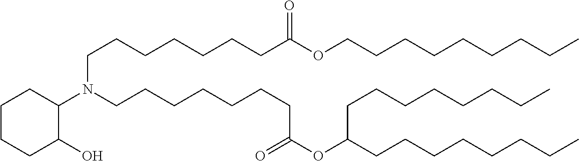 [SMILES: CCCCCCCCCOC(=O)CCCCCCCN(CCCCCCCC(=O)OC(CCCCCCCC)CCCCCCCC)C1CCCCC1O] |
| I 23 | 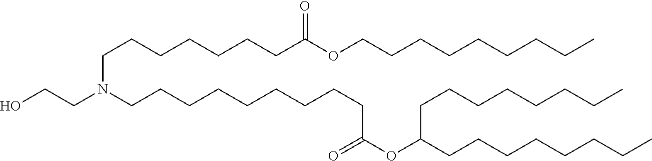 [SMILES: CCCCCCCCCOC(=O)CCCCCCCN(CCO)CCCCCCCCCC(=O)OC(CCCCCCCC)CCCCCCCC] |
| I 24 | 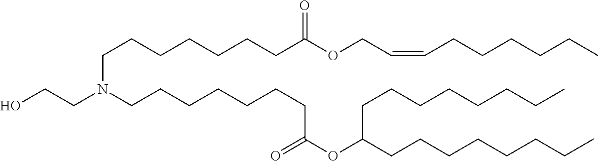 [SMILES: CCCCCC/C=C\COC(=O)CCCCCCCN(CCO)CCCCCCCC(=O)OC(CCCCCCCC)CCCCCCCC] |
| I 25 | 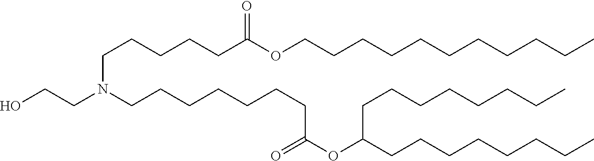 [SMILES: CCCCCCCCCCCOC(=O)CCCCCN(CCO)CCCCCCCC(=O)OC(CCCCCCCC)CCCCCCCC] |
| I 26 | 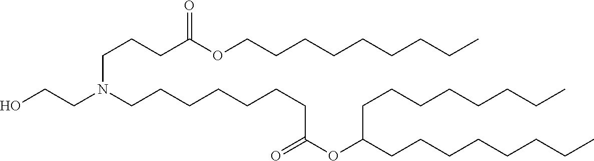 [SMILES: CCCCCCCCCOC(=O)CCCN(CCO)CCCCCCCC(=O)OC(CCCCCCCC)CCCCCCCC] |
| I 27 | 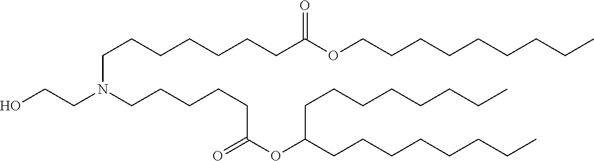 [SMILES: CCCCCCCCCOC(=O)CCCCCCCN(CCO)CCCCCC(=O)OC(CCCCCCCC)CCCCCCCC] |
| I 28 | 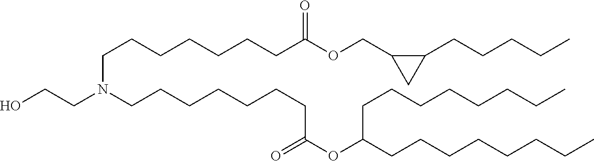 [SMILES: CCCCCCCCC(CCCCCCCC)OC(=O)CCCCCCCN(CCO)CCCCCCCC(=O)OCC1CC1CCCCC] |
| I 29 | 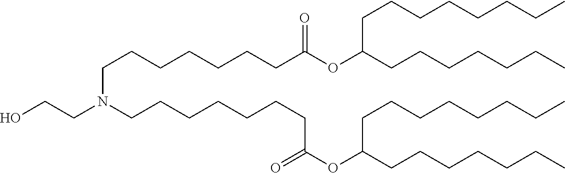 [SMILES: CCCCCCCCC(CCCCCCC)OC(=O)CCCCCCCN(CCO)CCCCCCCC(=O)OC(CCCCCCC)CCCCCCCC] |
| I 30 | 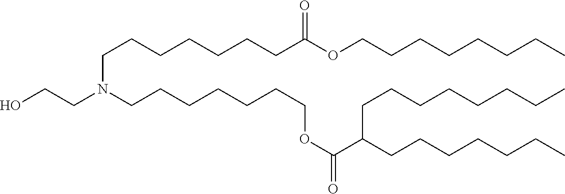 [SMILES: CCCCCCCCOC(=O)CCCCCCCN(CCO)CCCCCCCOC(=O)C(CCCCCCC)CCCCCCCC] |
| I 31 | 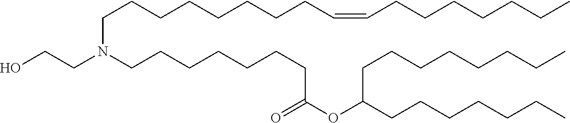 [SMILES: CCCCCCC/C=C\CCCCCCCCN(CCO)CCCCCCCC(=O)OC(CCCCCCC)CCCCCCCC] |
| I 32 | 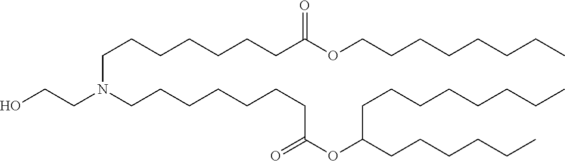 [SMILES: CCCCCCCCOC(=O)CCCCCCCN(CCO)CCCCCCCC(=O)OC(CCCCCC)CCCCCCCC] |
| I 33 | 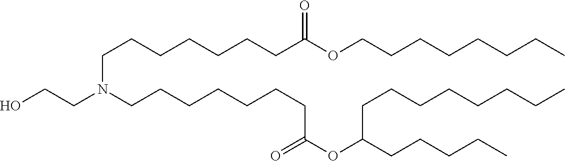 [SMILES: CCCCCCCCOC(=O)CCCCCCCN(CCO)CCCCCCCC(=O)OC(CCCCC)CCCCCCCC] |
| I 34 | 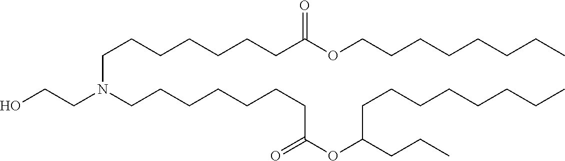 [SMILES: CCCCCCCCOC(=O)CCCCCCCN(CCO)CCCCCCCC(=O)OC(CCC)CCCCCCCC] |
| I 35 | 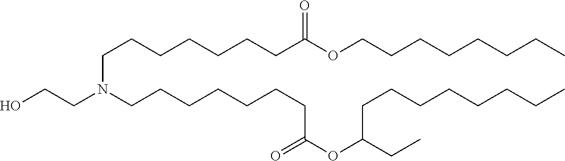 [SMILES: CCCCCCCCOC(=O)CCCCCCCN(CCO)CCCCCCCC(=O)OC(CC)CCCCCCCC] |
| I 36 | 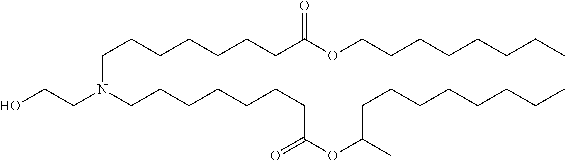 [SMILES: CCCCCCCCOC(=O)CCCCCCCN(CCO)CCCCCCCC(=O)OC(C)CCCCCCCC] |
| I 37 | 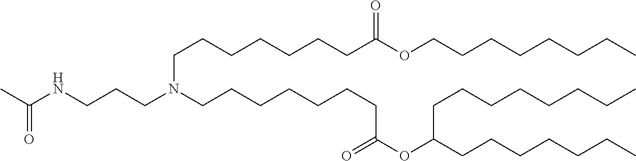 [SMILES: CCCCCCCCOC(=O)CCCCCCCN(CCCCCCCC(=O)OC(CCCCCCC)CCCCCCCC)CCCNC(C)=O] |
| I 38 | 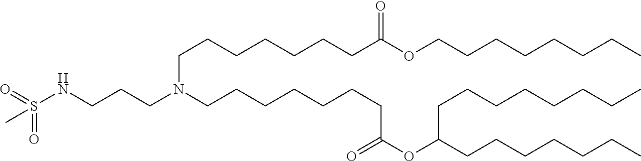 [SMILES: CCCCCCCCOC(=O)CCCCCCCN(CCCCCCCC(=O)OC(CCCCCCC)CCCCCCCC)CCCNS(C)(=O)=O] |
| I 39 | 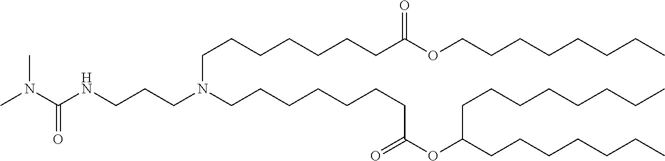 [SMILES: CCCCCCCCOC(=O)CCCCCCCN(CCCCCCCC(=O)OC(CCCCCCC)CCCCCCCC)CCCNC(=O)N(C)C] |
| I 40 | 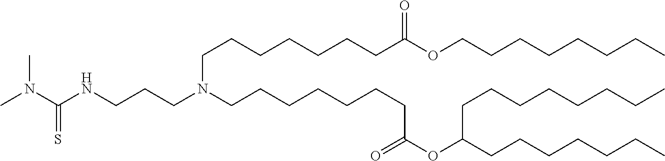 [SMILES: CCCCCCCCOC(=O)CCCCCCCN(CCCCCCCC(=O)OC(CCCCCCC)CCCCCCCC)CCCNC(=S)N(C)C] |
| I 41 | 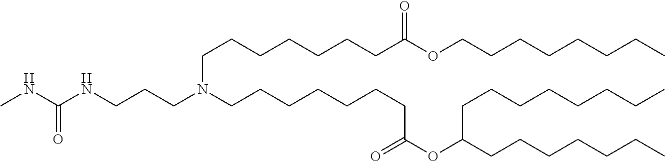 [SMILES: CCCCCCCCOC(=O)CCCCCCCN(CCCCCCCC(=O)OC(CCCCCCC)CCCCCCCC)CCCNC(=O)NC] |
| I 42 | 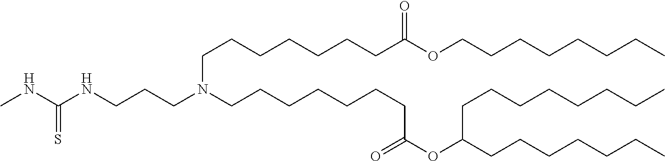 [SMILES: CCCCCCCCOC(=O)CCCCCCCN(CCCCCCCC(=O)OC(CCCCCCC)CCCCCCCC)CCCNC(=S)NC] |
| I 43 | 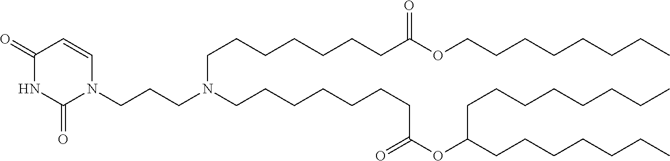 [SMILES: CCCCCCCCOC(=O)CCCCCCCN(CCCCCCCC(=O)OC(CCCCCCC)CCCCCCCC)CCCn1ccc(=O)[nH]c1=O] |
| I 44 | 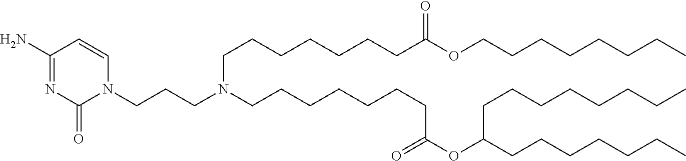 [SMILES: CCCCCCCCOC(=O)CCCCCCCN(CCCCCCCC(=O)OC(CCCCCCC)CCCCCCCC)CCCn1ccc(N)nc1=O] |
| I 45 | 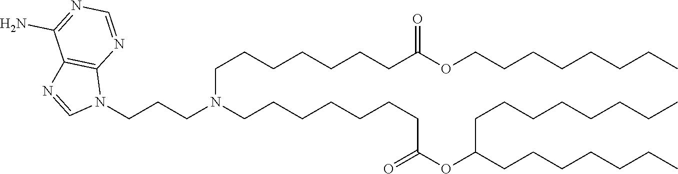 [SMILES: CCCCCCCCOC(=O)CCCCCCCN(CCCCCCCC(=O)OC(CCCCCCC)CCCCCCCC)CCCn1cnc2c(N)ncnc21] |
| I 46 | 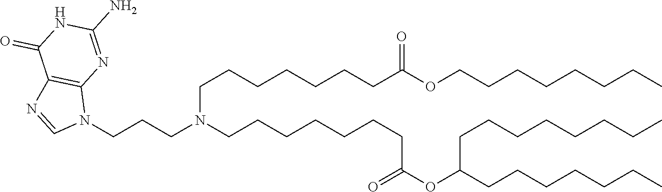 [SMILES: CCCCCCCCOC(=O)CCCCCCCN(CCCCCCCC(=O)OC(CCCCCCC)CCCCCCCC)CCCn1cnc2c(=O)[nH]c(N)nc21] |
| I 47 | 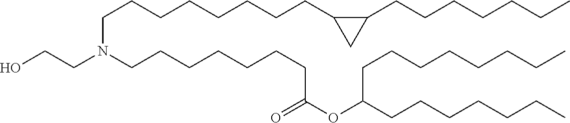 [SMILES: CCCCCCCCC(CCCCCCC)OC(=O)CCCCCCCN(CCO)CCCCCCCCC1CC1CCCCCCC] |
| I 48 | 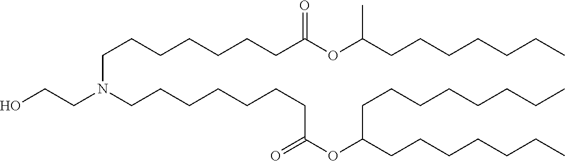 [SMILES: CCCCCCCCC(CCCCCCC)OC(=O)CCCCCCCN(CCO)CCCCCCCC(=O)OC(C)CCCCCCC] |
| I 49 | 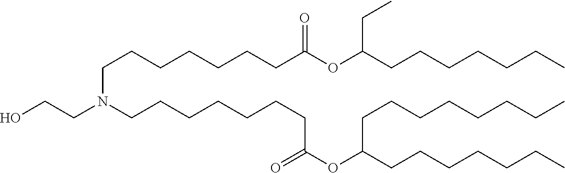 [SMILES: CCCCCCCCC(CCCCCCC)OC(=O)CCCCCCCN(CCO)CCCCCCCC(=O)OC(CC)CCCCCCC] |
| I 50 | 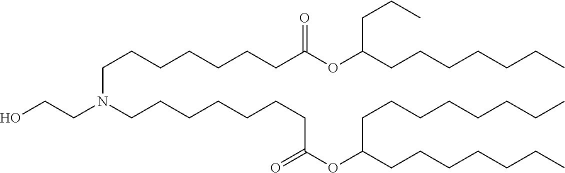 [SMILES: CCCCCCCCC(CCCCCCC)OC(=O)CCCCCCCN(CCO)CCCCCCCC(=O)OC(CCC)CCCCCCC] |
| I 51 | 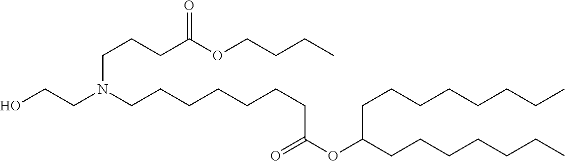 [SMILES: CCCCCCCCC(CCCCCCC)OC(=O)CCCCCCCN(CCO)CCCC(=O)OCCCC] |
| I 52 | 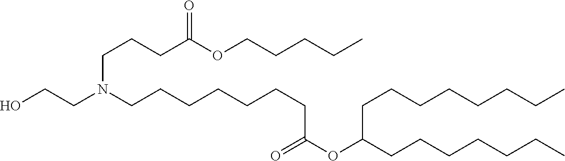 [SMILES: CCCCCCCCC(CCCCCCC)OC(=O)CCCCCCCN(CCO)CCCC(=O)OCCCCC] |
| I 53 | 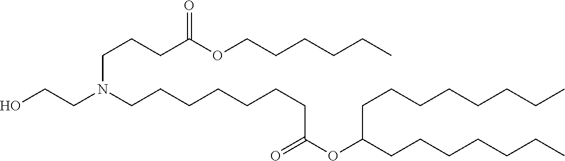 [SMILES: CCCCCCCCC(CCCCCCC)OC(=O)CCCCCCCN(CCO)CCCC(=O)OCCCCCC] |
| I 54 | 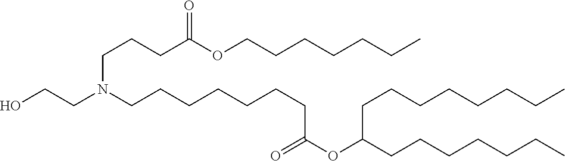 [SMILES: CCCCCCCCC(CCCCCCC)OC(=O)CCCCCCCN(CCO)CCCC(=O)OCCCCCCC] |
| I 55 | 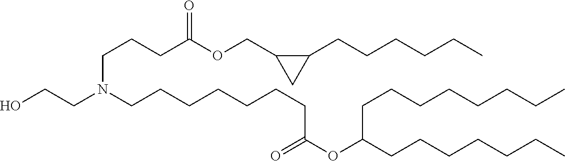 [SMILES: CCCCCCCCC(CCCCCCC)OC(=O)CCCCCCCN(CCO)CCCC(=O)OCC1CC1CCCCCC] |
| I 56 | 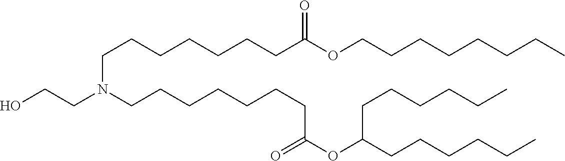 [SMILES: CCCCCCCCOC(=O)CCCCCCCN(CCO)CCCCCCCC(=O)OC(CCCCCC)CCCCCC] |
| I 57 | 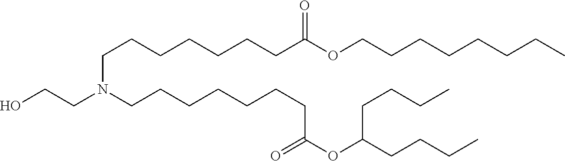 [SMILES: CCCCCCCCOC(=O)CCCCCCCN(CCO)CCCCCCCC(=O)OC(CCCC)CCCC] |
| I 58 | 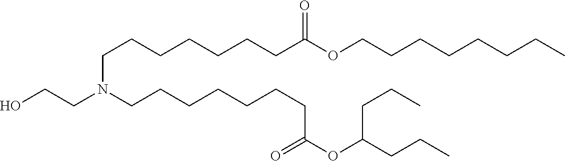 [SMILES: CCCCCCCCOC(=O)CCCCCCCN(CCO)CCCCCCCC(=O)OC(CCC)CCC] |
| I 59 | 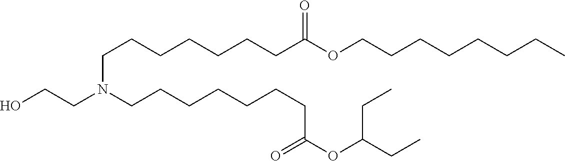 [SMILES: CCCCCCCCOC(=O)CCCCCCCN(CCO)CCCCCCCC(=O)OC(CC)CC] |
| I 60 | 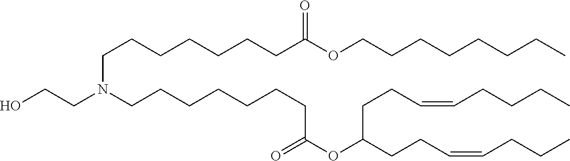 [SMILES: CCC/C=C\CCC(CC/C=C\CCCC)OC(=O)CCCCCCCN(CCO)CCCCCCCC(=O)OCCCCCCCC] |
| I 61 | 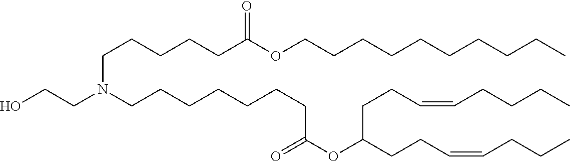 [SMILES: CCC/C=C\CCC(CC/C=C\CCCC)OC(=O)CCCCCCCN(CCO)CCCCCC(=O)OCCCCCCCCCC] |

In further embodiments, the compound of Formula (I I) is selected from the group consisting of:

| Cpd | Structure |
|---|---|
| I 62 | 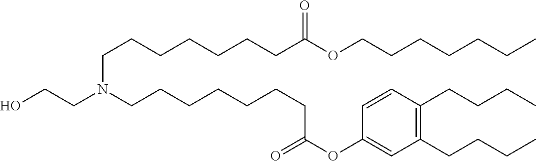 [SMILES: CCCCCCCOC(=O)CCCCCCCN(CCO)CCCCCCCC(=O)Oc1ccc(CCCC)c(CCCC)c1] |
| I 63 | 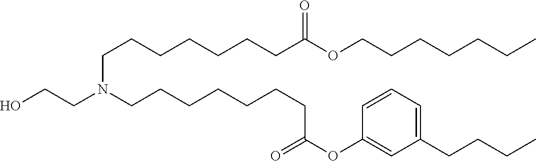 [SMILES: CCCCCCCOC(=O)CCCCCCCN(CCO)CCCCCCCC(=O)Oc1cccc(CCCC)c1] |
| I 64 | 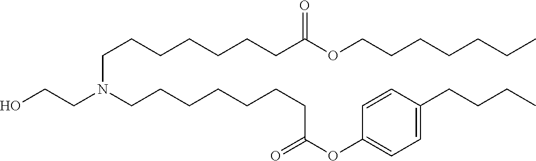 [SMILES: CCCCCCCOC(=O)CCCCCCCN(CCO)CCCCCCCC(=O)Oc1ccc(CCCC)cc1] |

In some embodiments, the compound of Formula (I I) or Formula (I IV) is selected from the group consisting of:

| Cpd | Structure |
|---|---|
| I 65 | 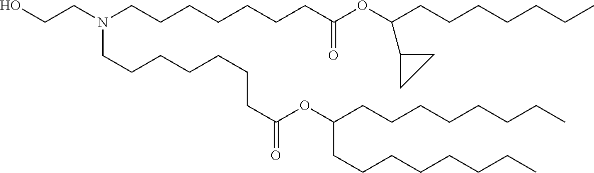 [SMILES: CCCCCCCCC(CCCCCCCC)OC(=O)CCCCCCCN(CCO)CCCCCCCC(=O)OC(CCCCCCC)C1CC1] |
| I 66 | 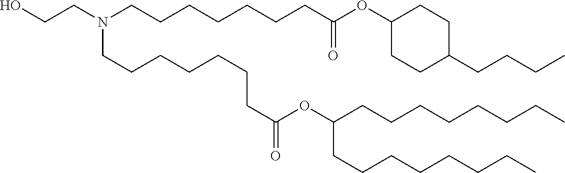 [SMILES: CCCCCCCCC(CCCCCCCC)OC(=O)CCCCCCCN(CCO)CCCCCCCC(=O)OC1CCC(CCCC)CC1] |
| I 67 | 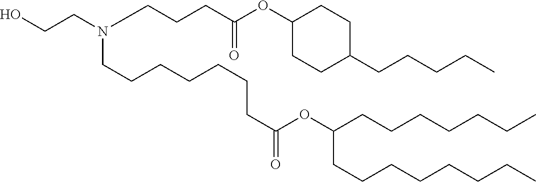 [SMILES: CCCCCCCCC(CCCCCCC)OC(=O)CCCCCCCN(CCO)CCCC(=O)OC1CCC(CCCCC)CC1] |
| I 68 | 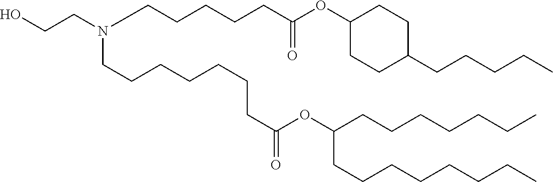 [SMILES: CCCCCCCCC(CCCCCCC)OC(=O)CCCCCCCN(CCO)CCCCCC(=O)OC1CCC(CCCCC)CC1] |
| I 69 | 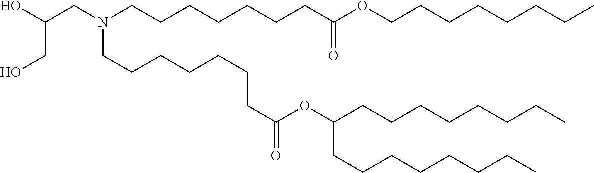 [SMILES: CCCCCCCCOC(=O)CCCCCCCN(CCCCCCCC(=O)OC(CCCCCCCC)CCCCCCCC)CC(O)CO] |
| I 70 | 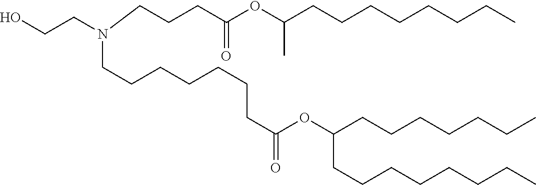 [SMILES: CCCCCCCCC(C)OC(=O)CCCN(CCO)CCCCCCCC(=O)OC(CCCCCCC)CCCCCCCC] |
| I 71 | 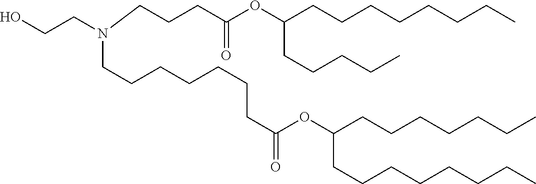 [SMILES: CCCCCCCCC(CCCCCCC)OC(=O)CCCCCCCN(CCO)CCCC(=O)OC(CCCCC)CCCCCCCC] |
| I 72 | 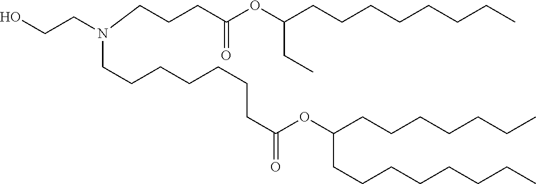 [SMILES: CCCCCCCCC(CC)OC(=O)CCCN(CCO)CCCCCCCC(=O)OC(CCCCCCC)CCCCCCCC] |
| I 73 | 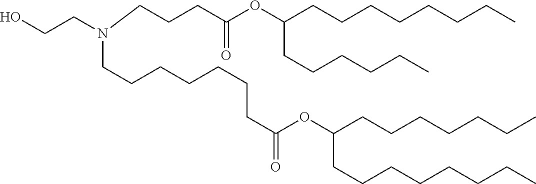 [SMILES: CCCCCCCCC(CCCCCCC)OC(=O)CCCCCCCN(CCO)CCCC(=O)OC(CCCCCC)CCCCCCCC] |
| I 74 | 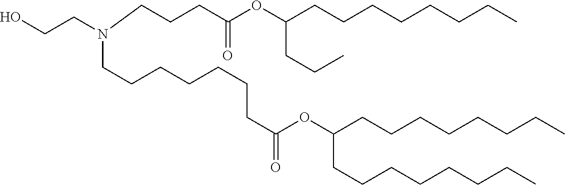 [SMILES: CCCCCCCCC(CCC)OC(=O)CCCN(CCO)CCCCCCCC(=O)OC(CCCCCCCC)CCCCCCCC] |
| I 75 | 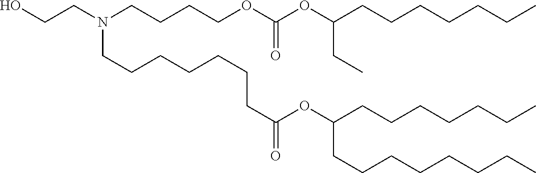 [SMILES: CCCCCCCCC(CCCCCCC)OC(=O)CCCCCCCN(CCO)CCCCOC(=O)OC(CC)CCCCCCC] |
| I 76 | 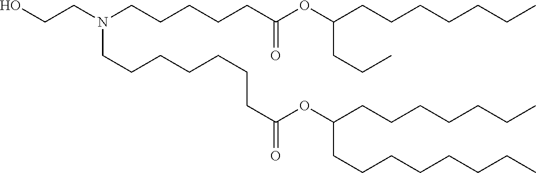 [SMILES: CCCCCCCCC(CCCCCCC)OC(=O)CCCCCCCN(CCO)CCCCCC(=O)OC(CCC)CCCCCCC] |
| I 77 | 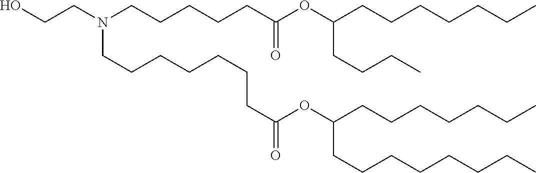 [SMILES: CCCCCCCCC(CCCCCCC)OC(=O)CCCCCCCN(CCO)CCCCCC(=O)OC(CCCC)CCCCCCC] |
| I 78 | 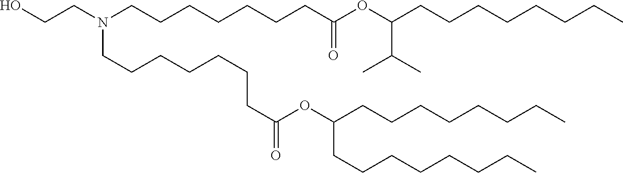 [SMILES: CCCCCCCCC(CCCCCCCC)OC(=O)CCCCCCCN(CCO)CCCCCCCC(=O)OC(CCCCCCCC)C(C)C] |
| I 79 | 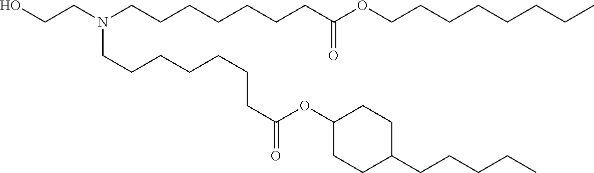 [SMILES: CCCCCCCCOC(=O)CCCCCCCN(CCO)CCCCCCCC(=O)OC1CCC(CCCCC)CC1] |
| I 80 | 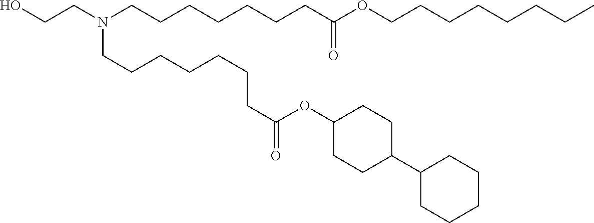 [SMILES: CCCCCCCCOC(=O)CCCCCCCN(CCO)CCCCCCCC(=O)OC1CCC(C2CCCCC2)CC1] |
| I 81 | 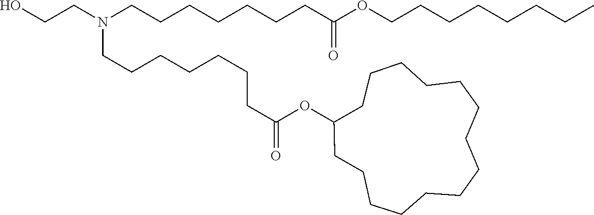 [SMILES: CCCCCCCCOC(=O)CCCCCCCN(CCO)CCCCCCCC(=O)OC1CCCCCCCCCCCCCC1] |
| I 82 | 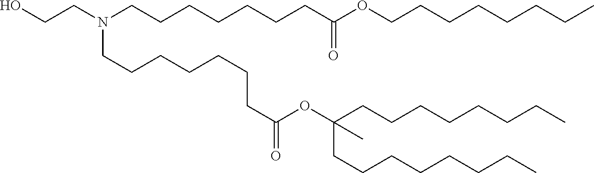 [SMILES: CCCCCCCCOC(=O)CCCCCCCN(CCO)CCCCCCCC(=O)OC(C)(CCCCCCCC)CCCCCCCC] |
| I 83 | 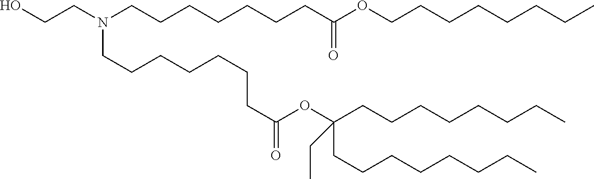 [SMILES: CCCCCCCCOC(=O)CCCCCCCN(CCO)CCCCCCCC(=O)OC(CC)(CCCCCCCC)CCCCCCCC] |
| I 84 | 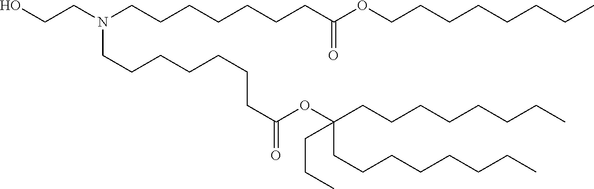 [SMILES: CCCCCCCCOC(=O)CCCCCCCN(CCO)CCCCCCCC(=O)OC(CCC)(CCCCCCCC)CCCCCCCC] |
| I 85 | 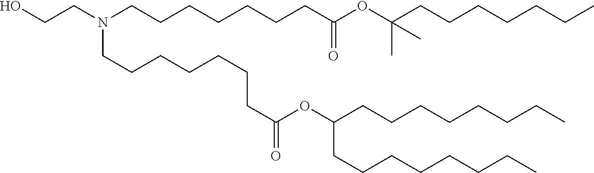 [SMILES: CCCCCCCCC(CCCCCCCC)OC(=O)CCCCCCCN(CCO)CCCCCCCC(=O)OC(C)(C)CCCCCCC] |
| I 86 | 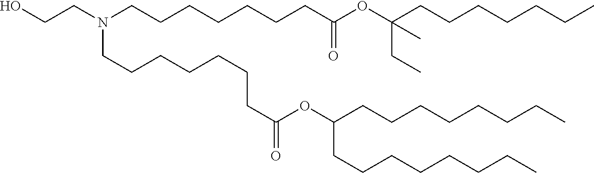 [SMILES: CCCCCCCCC(CCCCCCCC)OC(=O)CCCCCCCN(CCO)CCCCCCCC(=O)OC(C)(CC)CCCCCCC] |
| I 87 | 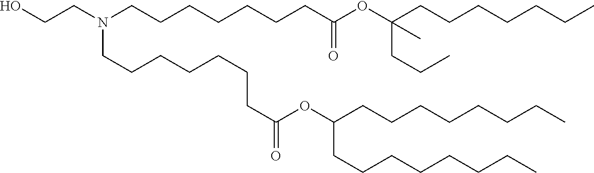 [SMILES: CCCCCCCCC(CCCCCCCC)OC(=O)CCCCCCCN(CCO)CCCCCCCC(=O)OC(C)(CCC)CCCCCCC] |
| I 88 | 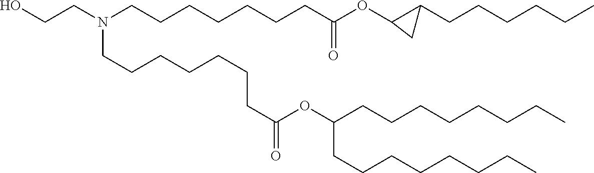 [SMILES: CCCCCCCCC(CCCCCCCC)OC(=O)CCCCCCCN(CCO)CCCCCCCC(=O)OC1CC1CCCCCC] |
| I 89 | 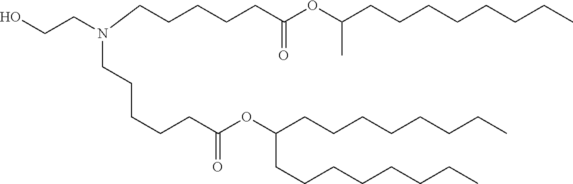 [SMILES: CCCCCCCCC(C)OC(=O)CCCCCN(CCO)CCCCCC(=O)OC(CCCCCCCC)CCCCCCCC] |
| I 90 | 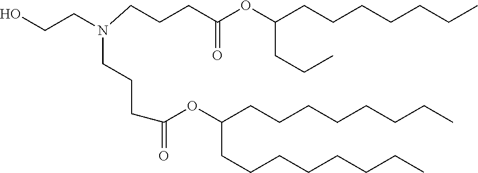 [SMILES: CCCCCCCCC(CCCCCCCC)OC(=O)CCCN(CCO)CCCC(=O)OC(CCC)CCCCCCC] |
| I 91 | 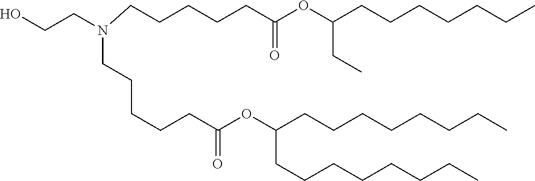 [SMILES: CCCCCCCCC(CCCCCCCC)OC(=O)CCCCCN(CCO)CCCCCC(=O)OC(CC)CCCCCCC] |
| I 92 | 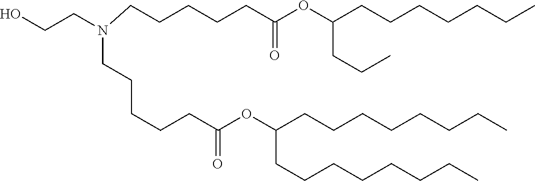 [SMILES: CCCCCCCCC(CCCCCCCC)OC(=O)CCCCCN(CCO)CCCCCC(=O)OC(CCC)CCCCCCC] |
| I 93 | 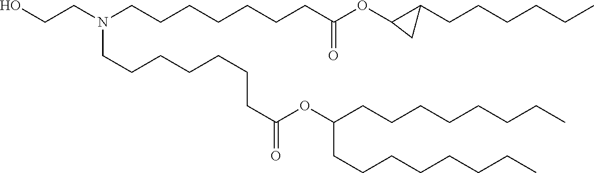 [SMILES: CCCCCCCCC(CCCCCCCC)OC(=O)CCCCCCCN(CCO)CCCCCCCC(=O)OC1CC1CCCCCC] |
| I 94 | 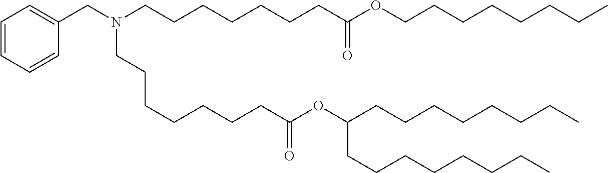 [SMILES: CCCCCCCCOC(=O)CCCCCCCN(CCCCCCCC(=O)OC(CCCCCCCC)CCCCCCCC)Cc1ccccc1] |
| I 95 | 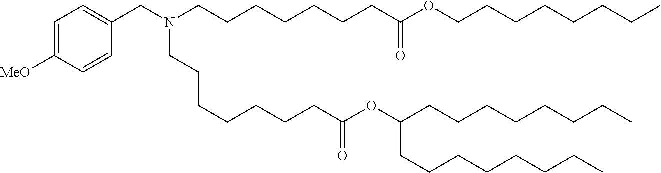 [SMILES: CCCCCCCCOC(=O)CCCCCCCN(CCCCCCCC(=O)OC(CCCCCCCC)CCCCCCCC)Cc1ccc(OC)cc1] |
| I 96 | 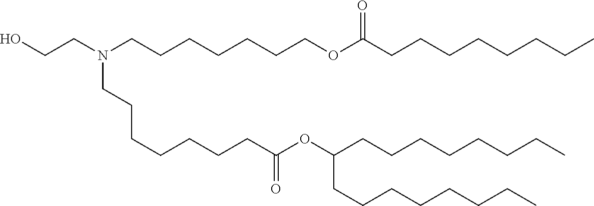 [SMILES: CCCCCCCCC(=O)OCCCCCCCN(CCO)CCCCCCCC(=O)OC(CCCCCCCC)CCCCCCCC] |
| I 97 | 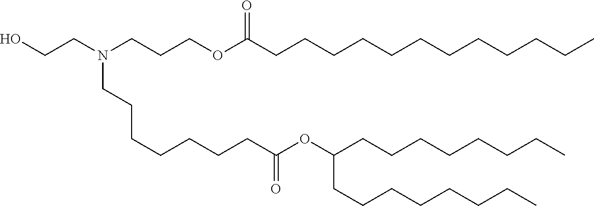 [SMILES: CCCCCCCCCCCCC(=O)OCCCN(CCO)CCCCCCCC(=O)OC(CCCCCCCC)CCCCCCCC] |
| I 98 | 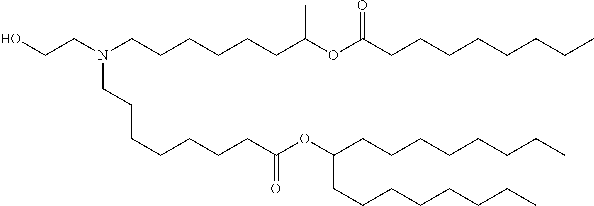 [SMILES: CCCCCCCCC(=O)OC(C)CCCCCCN(CCO)CCCCCCCC(=O)OC(CCCCCCCC)CCCCCCCC] |
| I 99 | 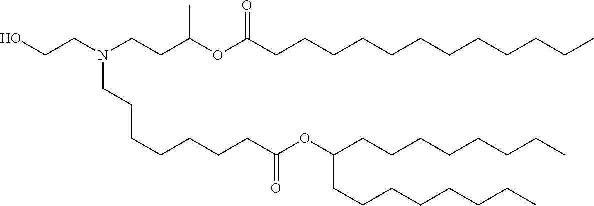 [SMILES: CCCCCCCCCCCCC(=O)OC(C)CCN(CCO)CCCCCCCC(=O)OC(CCCCCCCC)CCCCCCCC] |
| I 100 | 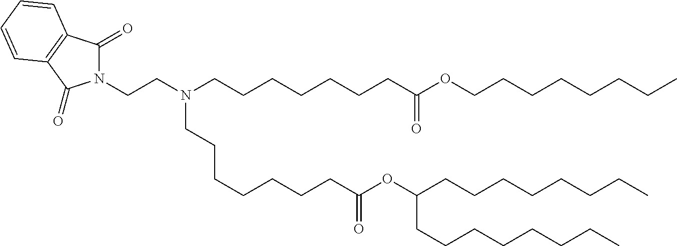 [SMILES: CCCCCCCCOC(=O)CCCCCCCN(CCCCCCCC(=O)OC(CCCCCCCC)CCCCCCCC)CCN1C(=O)c2ccccc2C1=O] |
| I 101 | 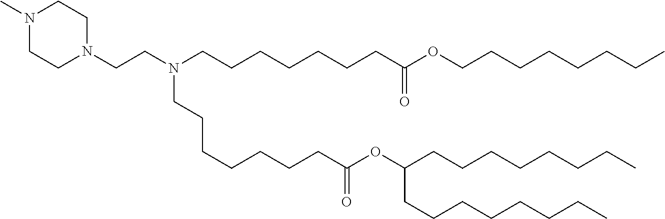 [SMILES: CCCCCCCCOC(=O)CCCCCCCN(CCCCCCCC(=O)OC(CCCCCCCC)CCCCCCCC)CCN1CCN(C)CC1] |
| I 102 | 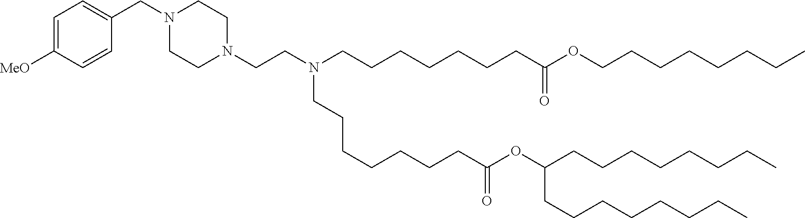 [SMILES: CCCCCCCCOC(=O)CCCCCCCN(CCCCCCCC(=O)OC(CCCCCCCC)CCCCCCCC)CCN1CCN(Cc2ccc(OC)cc2)CC1] |
| I 103 | 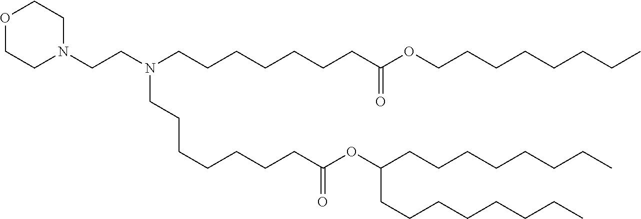 [SMILES: CCCCCCCCOC(=O)CCCCCCCN(CCCCCCCC(=O)OC(CCCCCCCC)CCCCCCCC)CCN1CCOCC1] |
| I 104 | 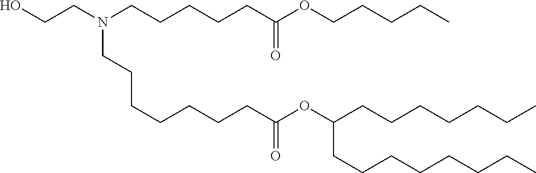 [SMILES: CCCCCCCCC(CCCCCCC)OC(=O)CCCCCCCN(CCO)CCCCCC(=O)OCCCCC] |
| I 105 | 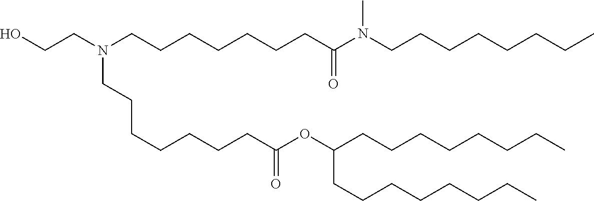 [SMILES: CCCCCCCCC(CCCCCCCC)OC(=O)CCCCCCCN(CCO)CCCCCCCC(=O)N(C)CCCCCCCC] |
| I 106 | 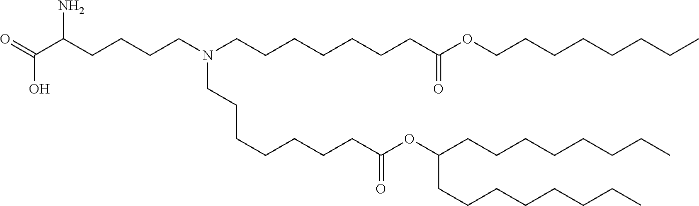 [SMILES: CCCCCCCCOC(=O)CCCCCCCN(CCCCCCCC(=O)OC(CCCCCCCC)CCCCCCCC)CCCCC(N)C(=O)O] |
| I 107 | 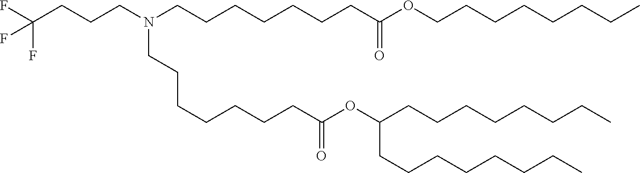 [SMILES: CCCCCCCCOC(=O)CCCCCCCN(CCCCCCCC(=O)OC(CCCCCCCC)CCCCCCCC)CCCC(F)(F)F] |
| I 108 | 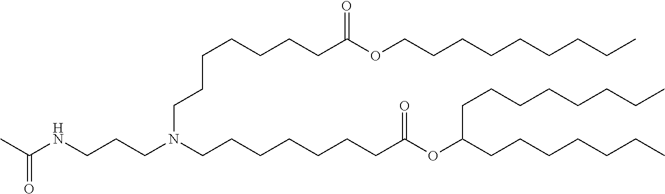 [SMILES: CCCCCCCCCOC(=O)CCCCCCCN(CCCCCCCC(=O)OC(CCCCCCC)CCCCCCCC)CCCNC(C)=O] |
| I 109 | 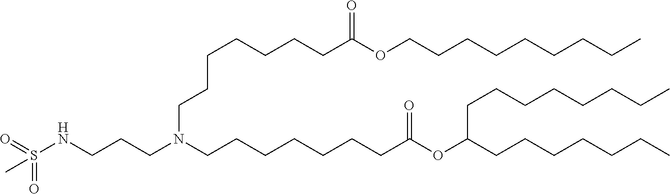 [SMILES: CCCCCCCCCOC(=O)CCCCCCCN(CCCCCCCC(=O)OC(CCCCCCC)CCCCCCCC)CCCNS(C)(=O)=O] |
| I 110 | 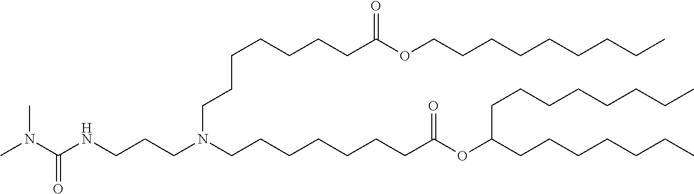 [SMILES: CCCCCCCCCOC(=O)CCCCCCCN(CCCCCCCC(=O)OC(CCCCCCC)CCCCCCCC)CCCNC(=O)N(C)C] |
| I 111 | 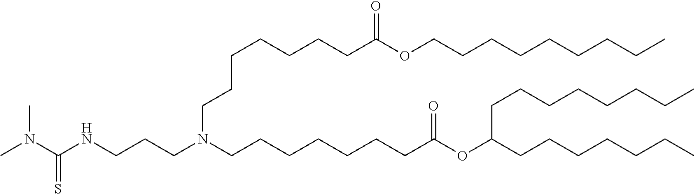 [SMILES: CCCCCCCCCOC(=O)CCCCCCCN(CCCCCCCC(=O)OC(CCCCCCC)CCCCCCCC)CCCNC(=S)N(C)C] |
| I 112 | 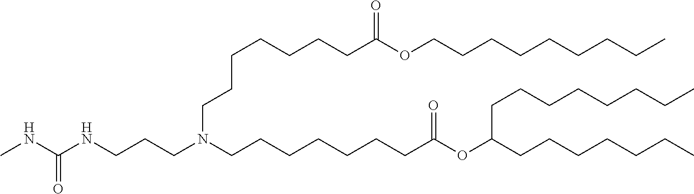 [SMILES: CCCCCCCCCOC(=O)CCCCCCCN(CCCCCCCC(=O)OC(CCCCCCC)CCCCCCCC)CCCNC(=O)NC] |
| I 113 | 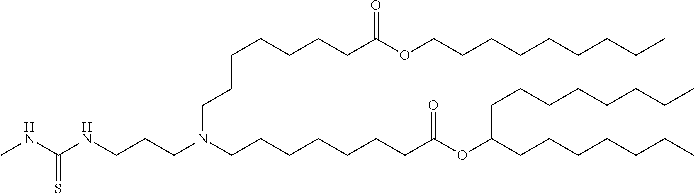 [SMILES: CCCCCCCCCOC(=O)CCCCCCCN(CCCCCCCC(=O)OC(CCCCCCC)CCCCCCCC)CCCNC(=S)NC] |
| I 114 | 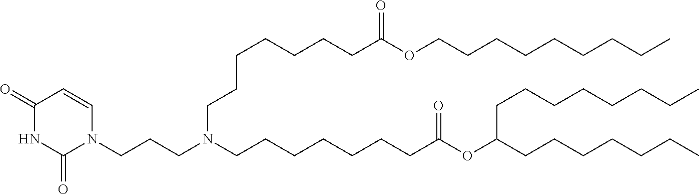 [SMILES: CCCCCCCCCOC(=O)CCCCCCCN(CCCCCCCC(=O)OC(CCCCCCC)CCCCCCCC)CCCn1ccc(=O)[nH]c1=O] |
| I 115 | 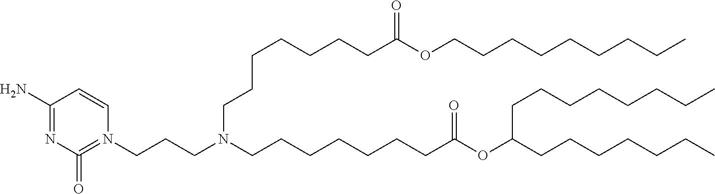 |
| I 116 | 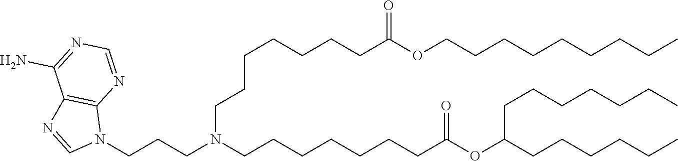 [SMILES: CCCCCCCCCOC(=O)CCCCCCCN(CCCCCCCC(=O)OC(CCCCCC)CCCCCCC)CCCn1cnc2c(N)ncnc21] |
| I 117 | 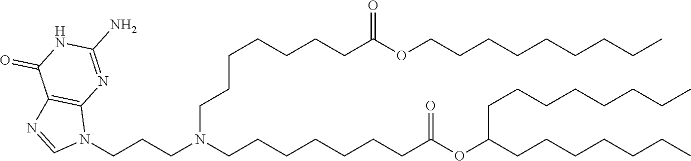 [SMILES: CCCCCCCCCOC(=O)CCCCCCCN(CCCCCCCC(=O)OC(CCCCCCC)CCCCCCCC)CCCn1cnc2c(=O)[nH]c(N)nc21] |
| I 118 | 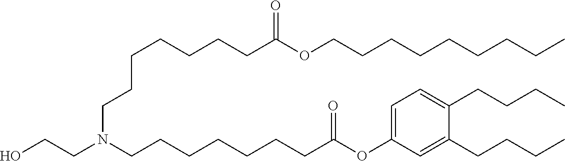 [SMILES: CCCCCCCCCOC(=O)CCCCCCCN(CCO)CCCCCCCC(=O)Oc1ccc(CCCC)c(CCCC)c1] |
| I 119 | 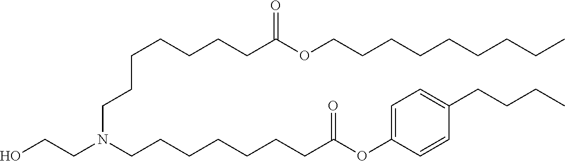 [SMILES: CCCCCCCCCOC(=O)CCCCCCCN(CCO)CCCCCCCC(=O)Oc1ccc(CCCC)cc1] |
| I 120 | 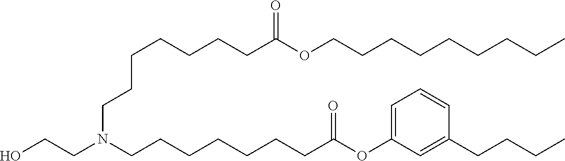 [SMILES: CCCCCCCCCOC(=O)CCCCCCCN(CCO)CCCCCCCC(=O)Oc1cccc(CCCC)c1] |
| I 121 | 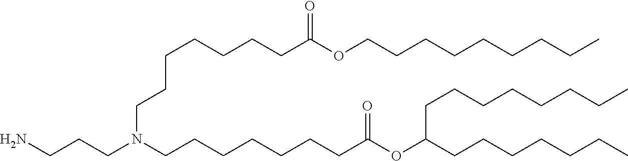 [SMILES: CCCCCCCCCOC(=O)CCCCCCCN(CCCN)CCCCCCCC(=O)OC(CCCCCCC)CCCCCCCC] |
| I 122 | 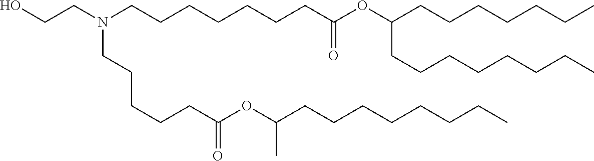 [SMILES: CCCCCCCCC(C)OC(=O)CCCCCN(CCO)CCCCCCCC(=O)OC(CCCCCCC)CCCCCCCC] |
| I 123 | 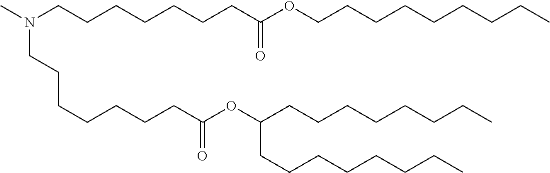 [SMILES: CCCCCCCCCOC(=O)CCCCCCCN(C)CCCCCCCC(=O)OC(CCCCCCCC)CCCCCCCC] |
| I 124 | 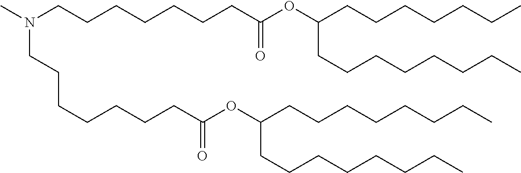 [SMILES: CCCCCCCCC(CCCCCCC)OC(=O)CCCCCCCN(C)CCCCCCCC(=O)OC(CCCCCCCC)CCCCCCCC] |
| I 125 | 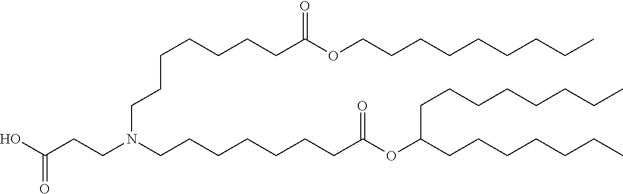 [SMILES: CCCCCCCCCOC(=O)CCCCCCCN(CCCCCCCC(=O)OC(CCCCCCC)CCCCCCCC)CCC(=O)O] |
| I 126 | 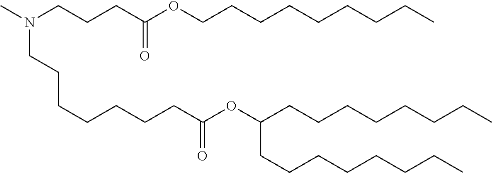 [SMILES: CCCCCCCCCOC(=O)CCCN(C)CCCCCCCC(=O)OC(CCCCCCCC)CCCCCCCC] |
| I 127 | 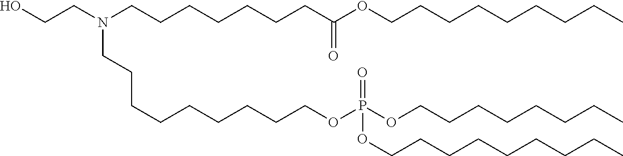 [SMILES: CCCCCCCCCOC(=O)CCCCCCCN(CCO)CCCCCCCCCOP(=O)(OCCCCCCCC)OCCCCCCCCC] |
| I 128 | 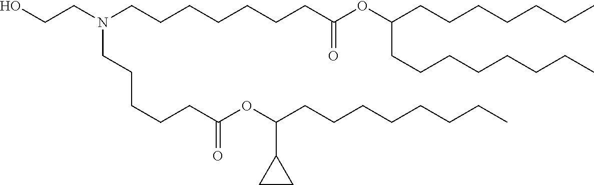 [SMILES: CCCCCCCCC(CCCCCCC)OC(=O)CCCCCCCN(CCO)CCCCCC(=O)OC(CCCCCCCC)C1CC1] |
| I 129 | 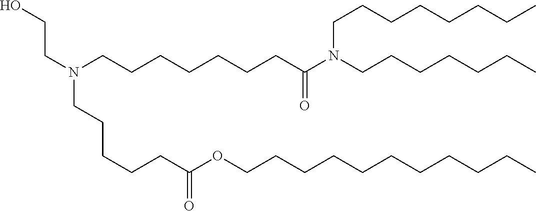 [SMILES: CCCCCCCCCCCOC(=O)CCCCCN(CCO)CCCCCCCC(=O)N(CCCCCCC)CCCCCCCC] |
| I 130 | 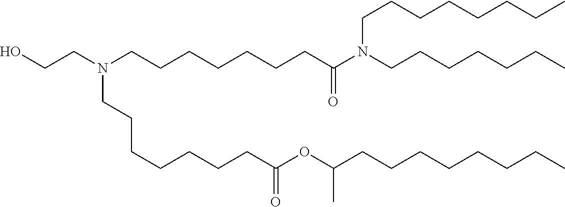 [SMILES: CCCCCCCCC(C)OC(=O)CCCCCCCN(CCO)CCCCCCCC(=O)N(CCCCCCC)CCCCCCCC] |
| I 131 | 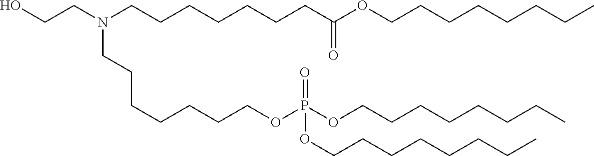 [SMILES: CCCCCCCCOC(=O)CCCCCCCN(CCO)CCCCCCCOP(=O)(OCCCCCCCC)OCCCCCCCC] |
| I 132 | 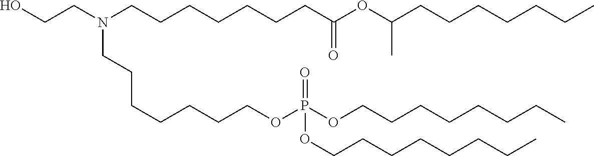 [SMILES: CCCCCCCCOP(=O)(OCCCCCCCC)OCCCCCCCN(CCO)CCCCCCCC(=O)OC(C)CCCCCCC] |
| I 133 | 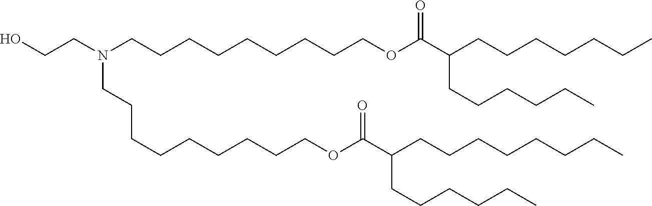 [SMILES: CCCCCCCCC(CCCCCC)C(=O)OCCCCCCCCCN(CCO)CCCCCCCCCOC(=O)C(CCCCCC)CCCCCCC] |
| I 134 | 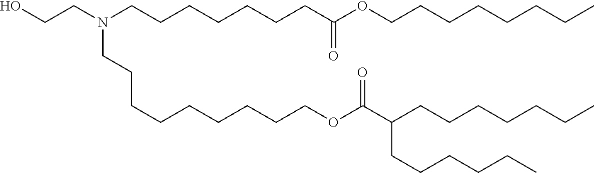 [SMILES: CCCCCCCCOC(=O)CCCCCCCN(CCO)CCCCCCCCCOC(=O)C(CCCCCC)CCCCCCC] |
| I 135 | 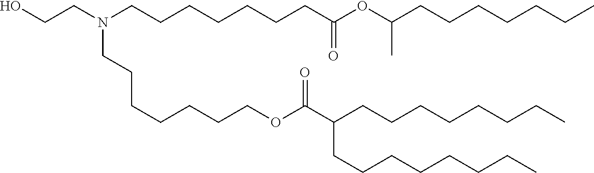 [SMILES: CCCCCCCCC(CCCCCCCC)C(=O)OCCCCCCCN(CCO)CCCCCCCC(=O)OC(C)CCCCCCC] |
| I 136 | 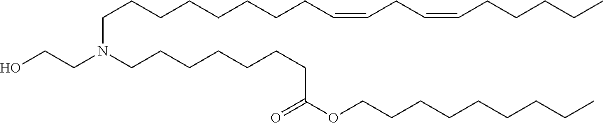 [SMILES: CCCCC/C=C\C/C=C\CCCCCCCCN(CCO)CCCCCCCC(=O)OCCCCCCCCC] |
| I 137 | 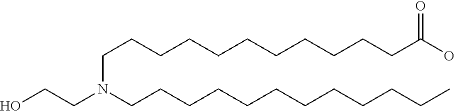 [SMILES: CCCCCCCCCCCCN(CCO)CCCCCCCCCCCC(=O)O] |
| I 138 | 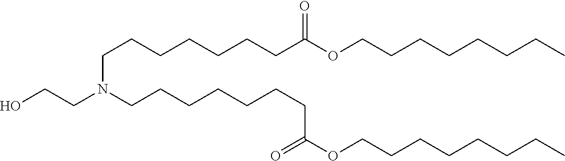 [SMILES: CCCCCCCCOC(=O)CCCCCCCN(CCO)CCCCCCCC(=O)OCCCCCCCC] |
| I 139 | 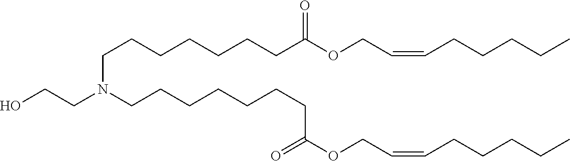 [SMILES: CCCCC/C=C\COC(=O)CCCCCCCN(CCO)CCCCCCCC(=O)OC/C=C\CCCCC] |
| I 140 | 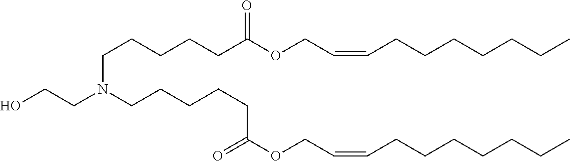 [SMILES: CCCCCCC/C=C\COC(=O)CCCCCN(CCO)CCCCCC(=O)OC/C=C\CCCCCCC] |
| I 141 | 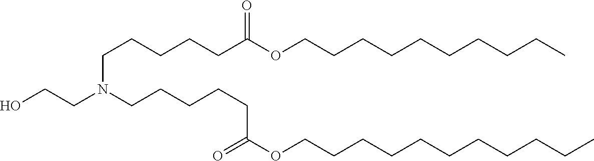 [SMILES: CCCCCCCCCCCOC(=O)CCCCCN(CCO)CCCCCC(=O)OCCCCCCCCCC] |
| I 142 | 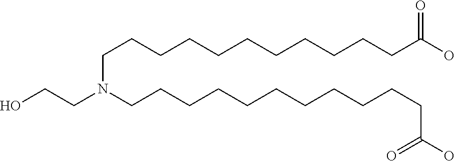 [SMILES: O=C(O)CCCCCCCCCCCN(CCO)CCCCCCCCCCCC(=O)O] |
| I 143 | 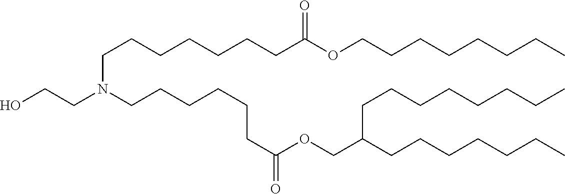 [SMILES: CCCCCCCCOC(=O)CCCCCCCN(CCO)CCCCCCC(=O)OCC(CCCCCCC)CCCCCCCC] |
| I 144 | 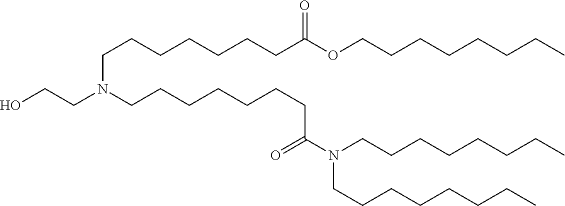 [SMILES: CCCCCCCCOC(=O)CCCCCCCN(CCO)CCCCCCCC(=O)N(CCCCCCCC)CCCCCCCC] |
| I 145 | 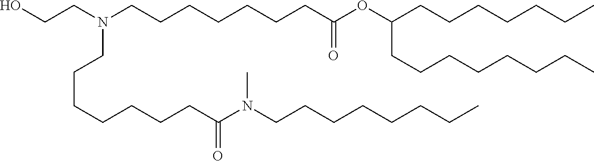 [SMILES: CCCCCCCCC(CCCCCCC)OC(=O)CCCCCCCN(CCO)CCCCCCCC(=O)N(C)CCCCCCCC] |
| I 146 | 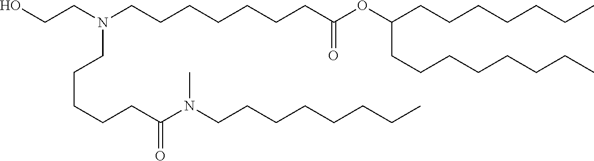 [SMILES: CCCCCCCCC(CCCCCCC)OC(=O)CCCCCCCN(CCO)CCCCCC(=O)N(C)CCCCCCCC] |
| I 147 | 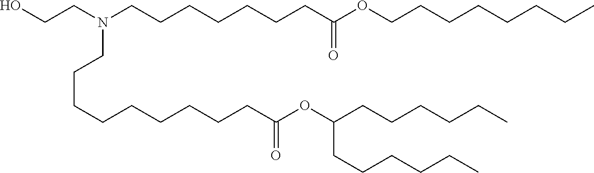 [SMILES: CCCCCCCCOC(=O)CCCCCCCN(CCO)CCCCCCCCCC(=O)OC(CCCCCC)CCCCCC] |
| I 148 | 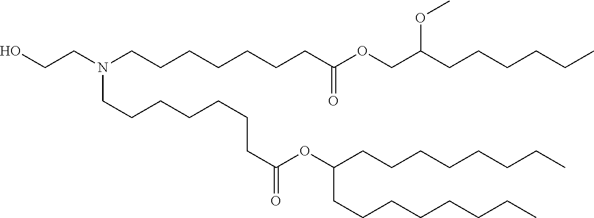 [SMILES: CCCCCCCCC(CCCCCCCC)OC(=O)CCCCCCCN(CCO)CCCCCCCC(=O)OCC(CCCCCC)OC] |
| I 149 | 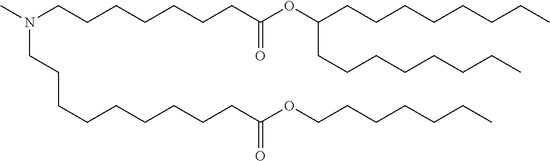 [SMILES: CCCCCCCCC(CCCCCCCC)OC(=O)CCCCCCCN(C)CCCCCCCCCC(=O)OCCCCCCC] |
| I 150 | 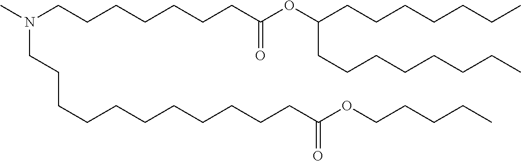 [SMILES: CCCCCCCCC(CCCCCCC)OC(=O)CCCCCCCN(C)CCCCCCCCCCCC(=O)OCCCCC] |
| I 151 | 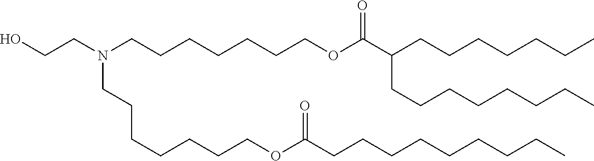 [SMILES: CCCCCCCCCC(=O)OCCCCCCCN(CCO)CCCCCCCOC(=O)C(CCCCCCC)CCCCCCCC] |
| I 152 | 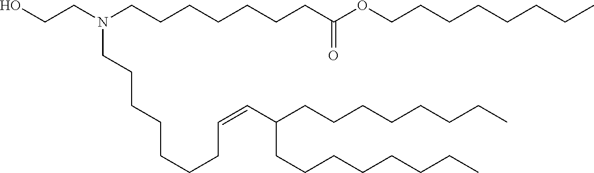 [SMILES: CCCCCCCCOC(=O)CCCCCCCN(CCO)CCCCCCC/C=C\C(CCCCCCCC)CCCCCCCC] |
| I 153 | 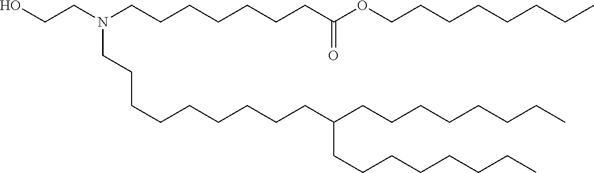 [SMILES: CCCCCCCCOC(=O)CCCCCCCN(CCO)CCCCCCCCCC(CCCCCCCC)CCCCCCCC] |
| I 154 | 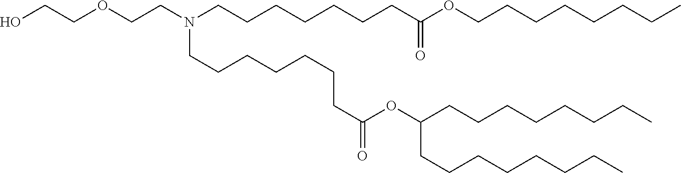 [SMILES: CCCCCCCCOC(=O)CCCCCCCN(CCCCCCCC(=O)OC(CCCCCCCC)CCCCCCCC)CCOCCO] |
| I 155 | 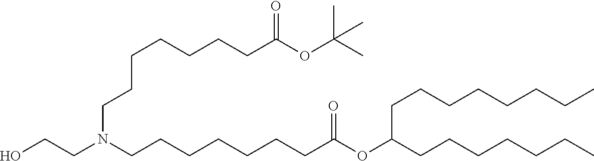 [SMILES: CCCCCCCCC(CCCCCCC)OC(=O)CCCCCCCN(CCO)CCCCCCCC(=O)OC(C)(C)C] |
| I 156 | 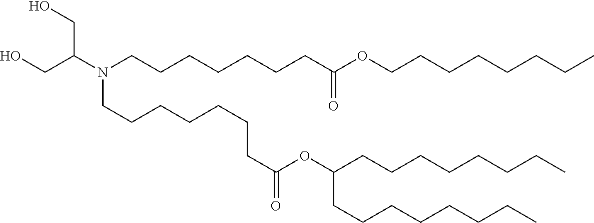 [SMILES: CCCCCCCCOC(=O)CCCCCCCN(CCCCCCCC(=O)OC(CCCCCCCC)CCCCCCCC)C(CO)CO] |
| I 157 | 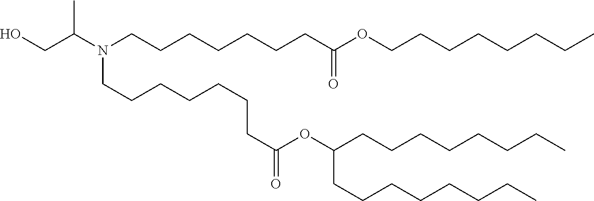 [SMILES: CCCCCCCCOC(=O)CCCCCCCN(CCCCCCCC(=O)OC(CCCCCCCC)CCCCCCCC)C(C)CO] |
| I 158 | 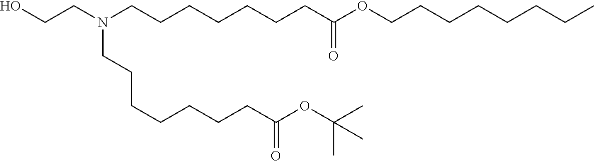 [SMILES: CCCCCCCCOC(=O)CCCCCCCN(CCO)CCCCCCCC(=O)OC(C)(C)C] |
| I 159 | 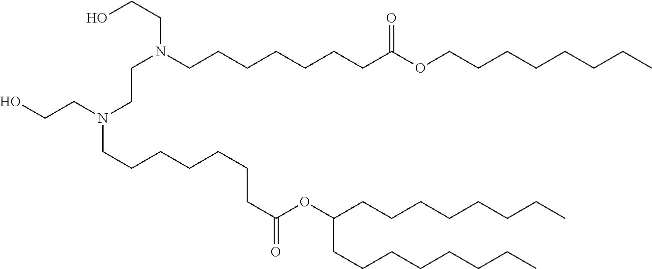 [SMILES: CCCCCCCCOC(=O)CCCCCCCN(CCO)CCN(CCO)CCCCCCCC(=O)OC(CCCCCCCC)CCCCCCCC] |
| I 160 | 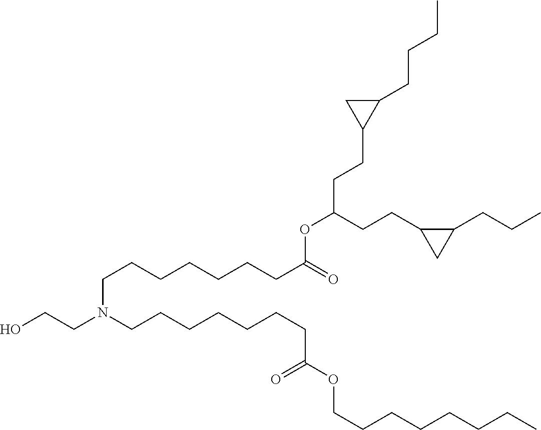 [SMILES: CCCCCCCCOC(=O)CCCCCCCN(CCO)CCCCCCCC(=O)OC(CCC1CC1CCC)CCC1CC1CCCC] |
| I 161 | 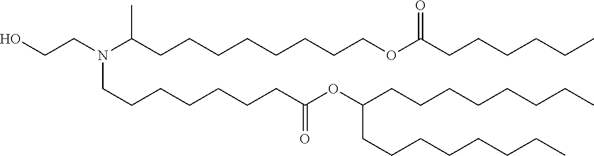 [SMILES: CCCCCCCCC(CCCCCCCC)OC(=O)CCCCCCCN(CCO)C(C)CCCCCCCCOC(=O)CCCCCC] |
| I 162 | 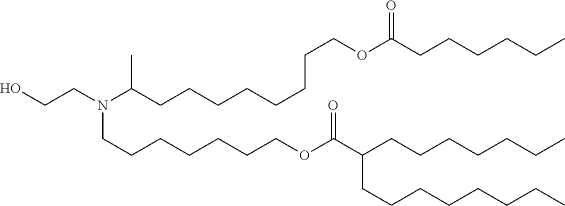 [SMILES: CCCCCCCCC(CCCCCCC)C(=O)OCCCCCCCN(CCO)C(C)CCCCCCCCOC(=O)CCCCCC] |
| I 163 | 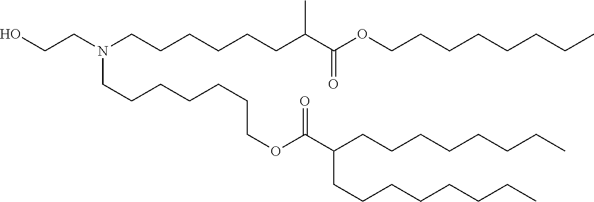 [SMILES: CCCCCCCCOC(=O)C(C)CCCCCCN(CCO)CCCCCCCOC(=O)C(CCCCCCCC)CCCCCCCC] |
| I 164 | 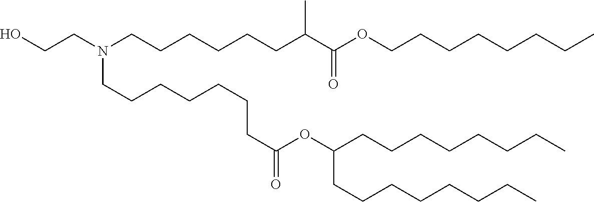 [SMILES: CCCCCCCCOC(=O)C(C)CCCCCCN(CCO)CCCCCCCC(=O)OC(CCCCCCCC)CCCCCCCC] |
| I 165 | 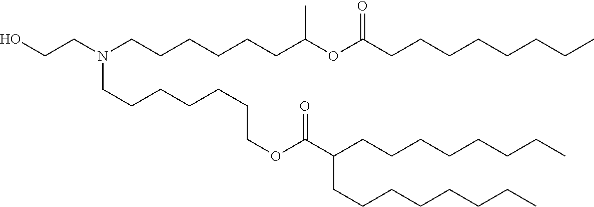 [SMILES: CCCCCCCCC(=O)OC(C)CCCCCCN(CCO)CCCCCCCOC(=O)C(CCCCCCCC)CCCCCCCC] |
| I 166 | 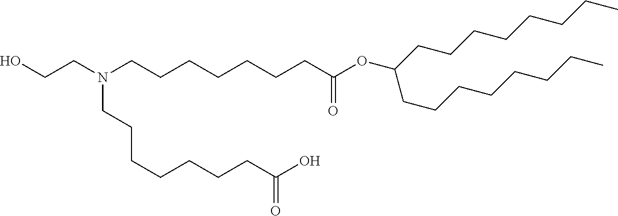 [SMILES: CCCCCCCCC(CCCCCCCC)OC(=O)CCCCCCCN(CCO)CCCCCCCC(=O)O] |
| I 167 | 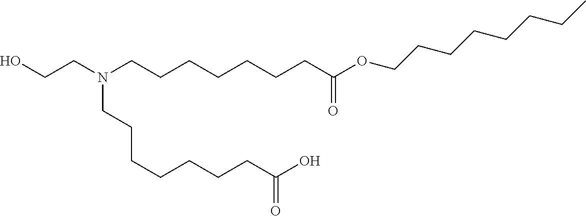 [SMILES: CCCCCCCCOC(=O)CCCCCCCN(CCO)CCCCCCCC(=O)O] |
| I 168 | 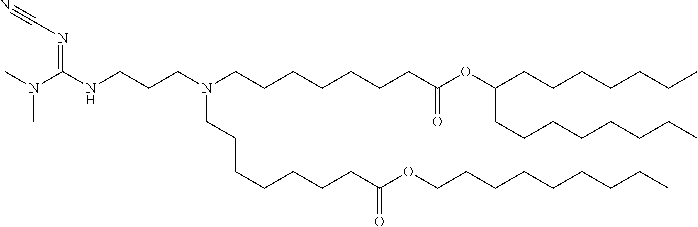 [SMILES: CCCCCCCCCOC(=O)CCCCCCCN(CCCCCCCC(=O)OC(CCCCCCC)CCCCCCCC)CCCN/C(=N/C#N)N(C)C] |
| I 169 | 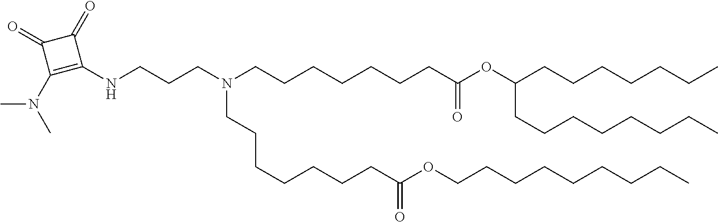 [SMILES: CCCCCCCCCOC(=O)CCCCCCCN(CCCCCCCC(=O)OC(CCCCCCC)CCCCCCCC)CCCNc1c(N(C)C)c(=O)c1=O] |
| I 170 | 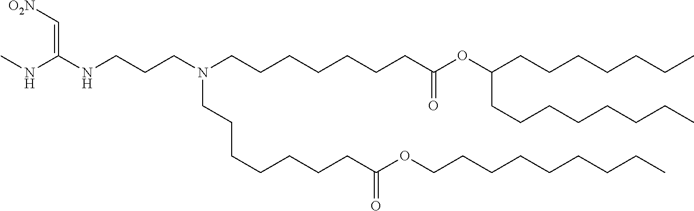 [SMILES: CCCCCCCCCOC(=O)CCCCCCCN(CCCCCCCC(=O)OC(CCCCCCC)CCCCCCCC)CCCN/C(=C/[N+](=O)[O-])NC] |
| I 171 | 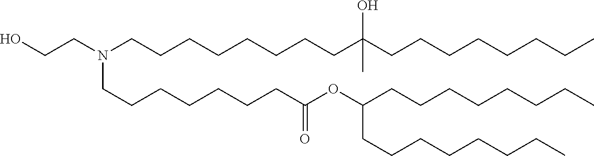 [SMILES: CCCCCCCCC(CCCCCCCC)OC(=O)CCCCCCCN(CCO)CCCCCCCCC(C)(O)CCCCCCCC] |
| I 172 | 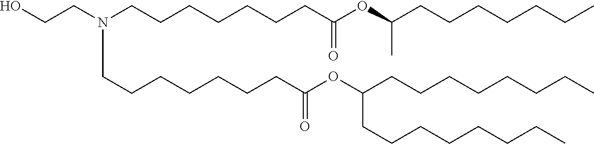 [SMILES: CCCCCCCCC(CCCCCCCC)OC(=O)CCCCCCCN(CCO)CCCCCCCC(=O)O[C@H](C)CCCCCCC] |
| I 173 | 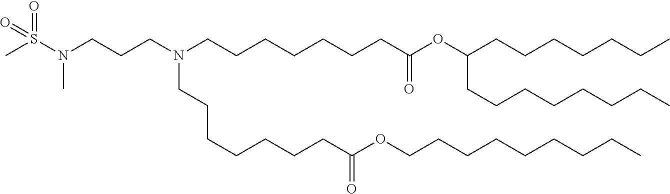 [SMILES: CCCCCCCCCOC(=O)CCCCCCCN(CCCCCCCC(=O)OC(CCCCCCC)CCCCCCCC)CCCN(C)S(C)(=O)=O] |
| I 174 | 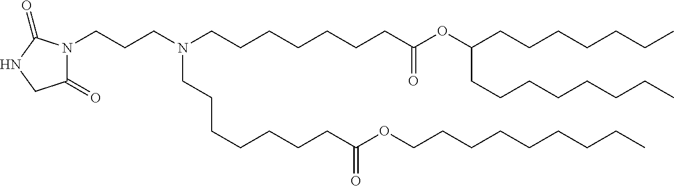 [SMILES: CCCCCCCCCOC(=O)CCCCCCCN(CCCCCCCC(=O)OC(CCCCCCC)CCCCCCCC)CCCN1C(=O)CNC1=O] |
| I 175 | 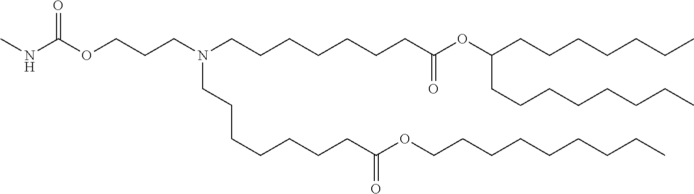 [SMILES: CCCCCCCCCOC(=O)CCCCCCCN(CCCCCCCC(=O)OC(CCCCCCC)CCCCCCCC)CCCOC(=O)NC] |
| I 176 | 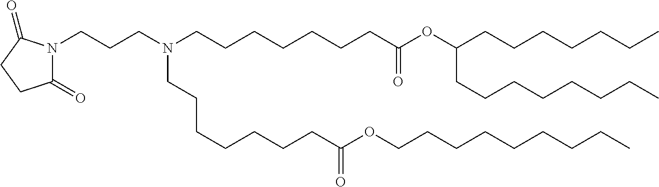 [SMILES: CCCCCCCCCOC(=O)CCCCCCCN(CCCCCCCC(=O)OC(CCCCCCC)CCCCCCCC)CCCN1C(=O)CCC1=O] |
| I 177 | 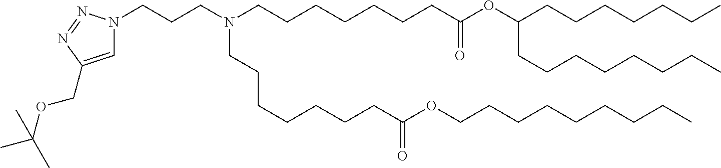 [SMILES: CCCCCCCCCOC(=O)CCCCCCCN(CCCCCCCC(=O)OC(CCCCCCC)CCCCCCCC)CCCn1cc(COC(C)(C)C)nn1] |
| I 178 | 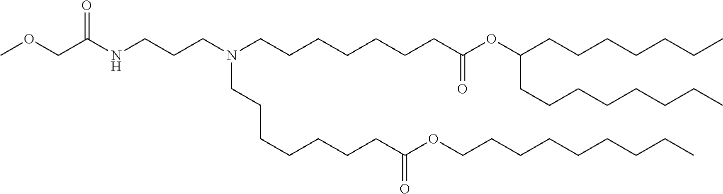 [SMILES: CCCCCCCCCOC(=O)CCCCCCCN(CCCCCCCC(=O)OC(CCCCCCC)CCCCCCCC)CCCNC(=O)COC] |
| I 179 | 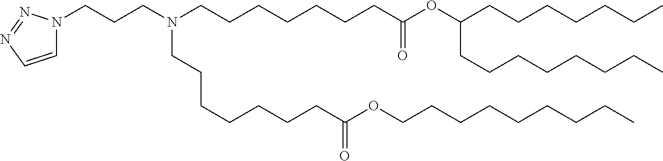 [SMILES: CCCCCCCCCOC(=O)CCCCCCCN(CCCCCCCC(=O)OC(CCCCCCC)CCCCCCCC)CCCn1ccnn1] |
| I 180 | 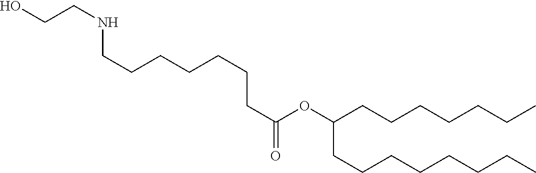 [SMILES: CCCCCCCCC(CCCCCCC)OC(=O)CCCCCCCNCCO] |
| I 181 | 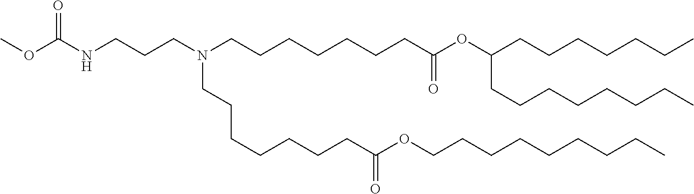 [SMILES: CCCCCCCCCOC(=O)CCCCCCCN(CCCCCCCC(=O)OC(CCCCCCC)CCCCCCCC)CCCNC(=O)OC] |
| I 182 | 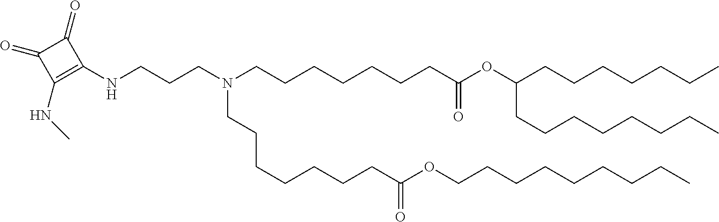 [SMILES: CCCCCCCCCOC(=O)CCCCCCCN(CCCCCCCC(=O)OC(CCCCCCC)CCCCCCCC)CCCNc1c(NC)c(=O)c1=O] |
| I 183 | 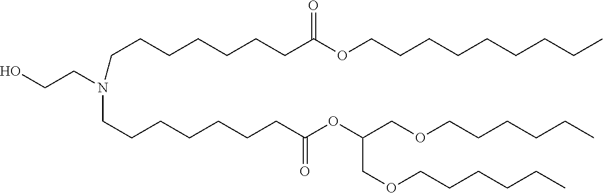 [SMILES: CCCCCCCCCOC(=O)CCCCCCCN(CCO)CCCCCCCC(=O)OC(COCCCCCC)COCCCCCC] |
| I 184 | 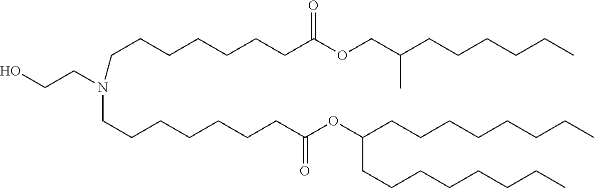 [SMILES: CCCCCCCCC(CCCCCCCC)OC(=O)CCCCCCCN(CCO)CCCCCCCC(=O)OCC(C)CCCCCC] |
| I 185 | 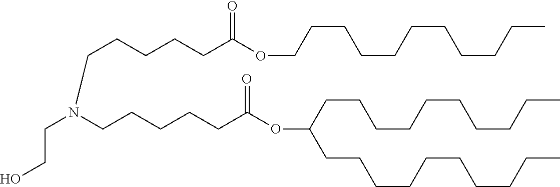 [SMILES: CCCCCCCCCCCOC(=O)CCCCCN(CCO)CCCCCC(=O)OC(CCCCCCCCCC)CCCCCCCCCC] |
| I 186 | 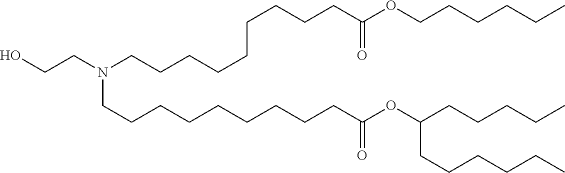 [SMILES: CCCCCCOC(=O)CCCCCCCCCN(CCO)CCCCCCCCCC(=O)OC(CCCCC)CCCCCC] |
| I 187 | 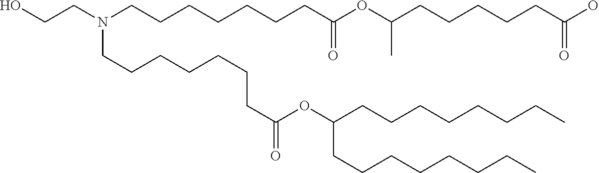 [SMILES: CCCCCCCCC(CCCCCCCC)OC(=O)CCCCCCCN(CCO)CCCCCCCC(=O)OC(C)CCCCCC(=O)O] |
| I 188 | 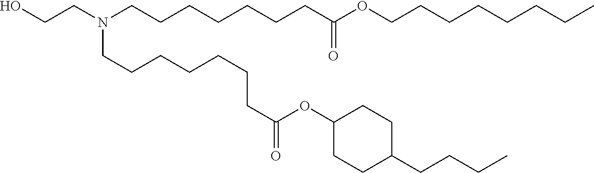 [SMILES: CCCCCCCCOC(=O)CCCCCCCN(CCO)CCCCCCCC(=O)OC1CCC(CCCC)CC1] |
| I 189 | 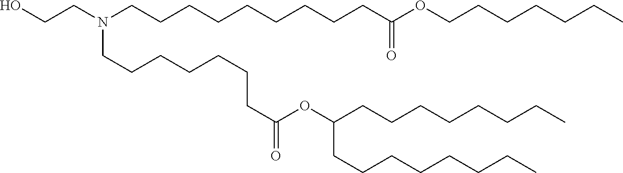 [SMILES: CCCCCCCCC(CCCCCCCC)OC(=O)CCCCCCCN(CCO)CCCCCCCCCC(=O)OCCCCCCC] |
| I 190 | 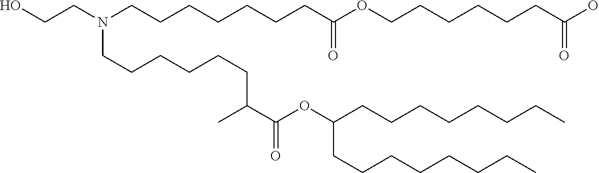 [SMILES: CCCCCCCCC(CCCCCCCC)OC(=O)C(C)CCCCCCN(CCO)CCCCCCCC(=O)OCCCCCCC(=O)O] |
| I 191 | 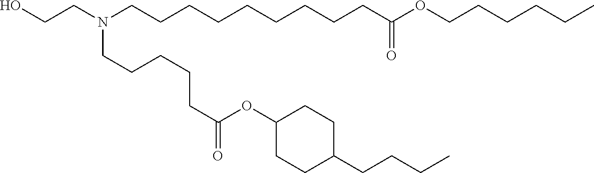 [SMILES: CCCCCCOC(=O)CCCCCCCCCN(CCO)CCCCCC(=O)OC1CCC(CCCC)CC1] |
| I 192 | 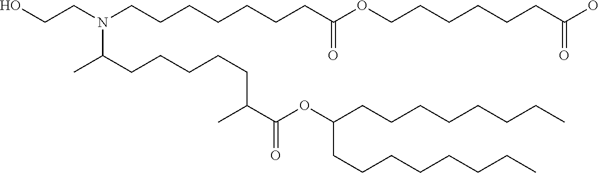 [SMILES: CCCCCCCCC(CCCCCCCC)OC(=O)C(C)CCCCCC(C)N(CCO)CCCCCCCC(=O)OCCCCCCC(=O)O] |
| I 193 | 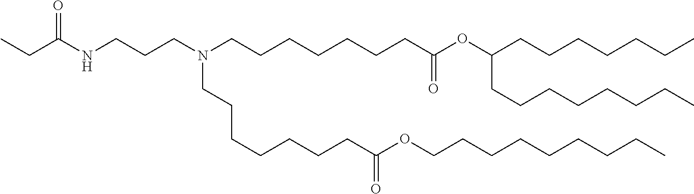 [SMILES: CCCCCCCCCOC(=O)CCCCCCCN(CCCCCCCC(=O)OC(CCCCCCC)CCCCCCCC)CCCNC(=O)CC] |
| I 194 | 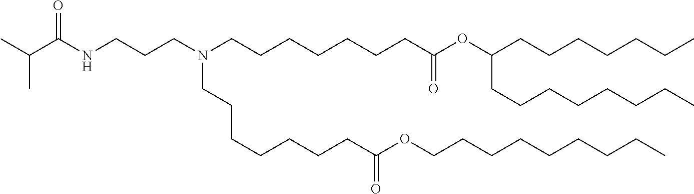 [SMILES: CCCCCCCCCOC(=O)CCCCCCCN(CCCCCCCC(=O)OC(CCCCCCC)CCCCCCCC)CCCNC(=O)C(C)C] |
| I 195 | 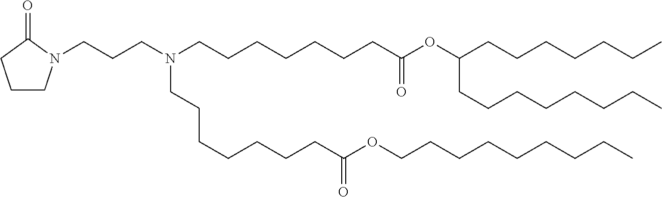 [SMILES: CCCCCCCCCOC(=O)CCCCCCCN(CCCCCCCC(=O)OC(CCCCCCC)CCCCCCCC)CCCN1CCCC1=O] |
| I 196 | 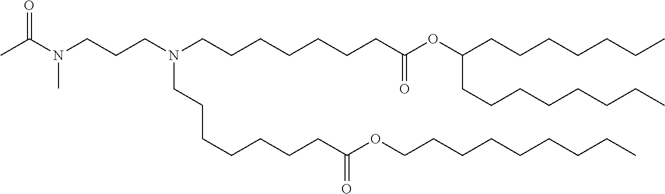 [SMILES: CCCCCCCCCOC(=O)CCCCCCCN(CCCCCCCC(=O)OC(CCCCCCC)CCCCCCCC)CCCN(C)C(C)=O] |
| I 197 | 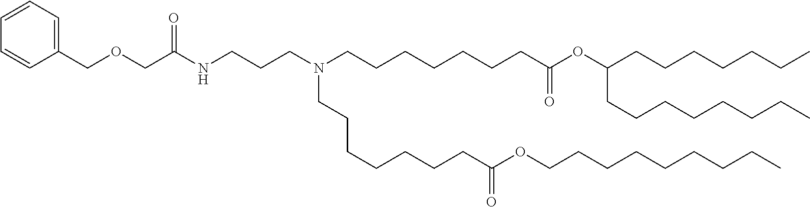 [SMILES: CCCCCCCCCOC(=O)CCCCCCCN(CCCCCCCC(=O)OC(CCCCCCC)CCCCCCCC)CCCNC(=O)COCc1ccccc1] |
| I 198 | 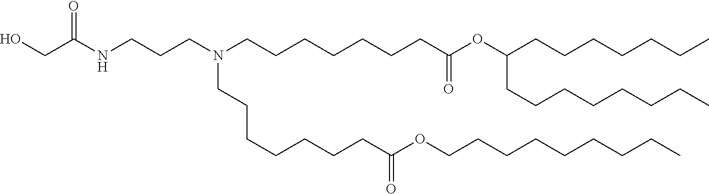 [SMILES: CCCCCCCCCOC(=O)CCCCCCCN(CCCCCCCC(=O)OC(CCCCCCC)CCCCCCCC)CCCNC(=O)CO] |
| I 199 | 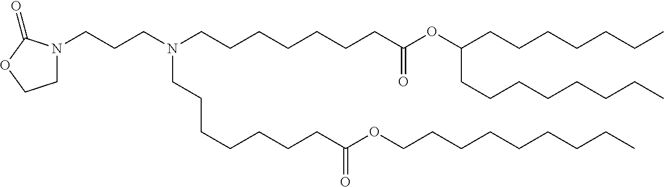 [SMILES: CCCCCCCCCOC(=O)CCCCCCCN(CCCCCCCC(=O)OC(CCCCCCC)CCCCCCCC)CCCN1CCOC1=O] |
| I 200 | 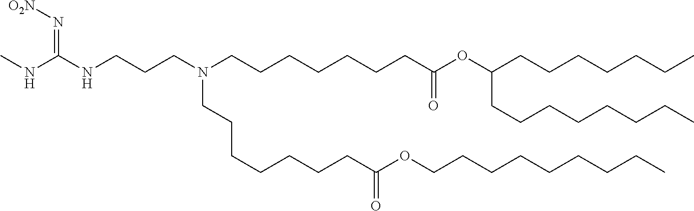 [SMILES: CCCCCCCCCOC(=O)CCCCCCCN(CCCCCCCC(=O)OC(CCCCCCC)CCCCCCCC)CCCN/C(=N/[N+](=O)[O-])NC] |
| I 201 | 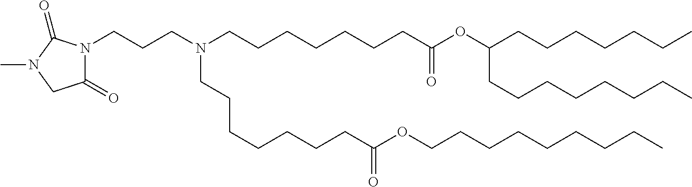 [SMILES: CCCCCCCCCOC(=O)CCCCCCCN(CCCCCCCC(=O)OC(CCCCCCC)CCCCCCCC)CCCN1C(=O)CN(C)C1=O] |
| I 202 | 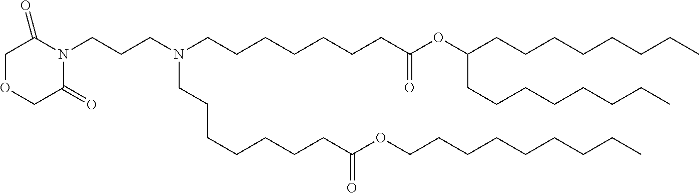 [SMILES: CCCCCCCCCOC(=O)CCCCCCCN(CCCCCCCC(=O)OC(CCCCCCCC)CCCCCCCC)CCCN1C(=O)COCC1=O] |
| I 203 | 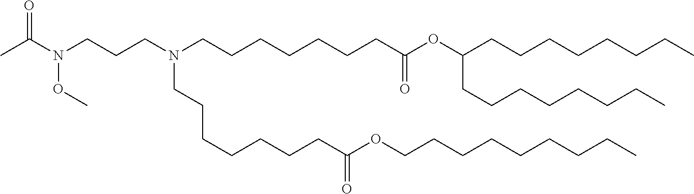 [SMILES: CCCCCCCCCOC(=O)CCCCCCCN(CCCCCCCC(=O)OC(CCCCCCCC)CCCCCCCC)CCCN(OC)C(C)=O] |
| I 204 | 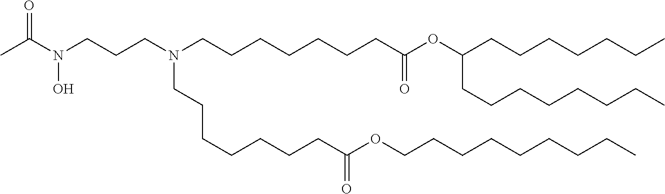 [SMILES: CCCCCCCCCOC(=O)CCCCCCCN(CCCCCCCC(=O)OC(CCCCCCC)CCCCCCCC)CCCN(O)C(C)=O] |
| I 205 | 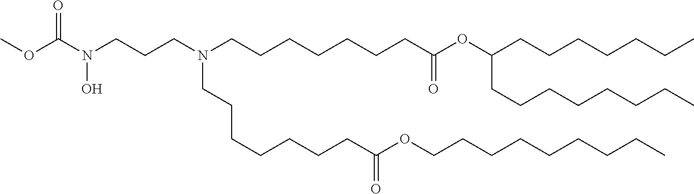 [SMILES: CCCCCCCCCOC(=O)CCCCCCCN(CCCCCCCC(=O)OC(CCCCCCC)CCCCCCCC)CCCN(O)C(=O)OC] |
| I 206 | 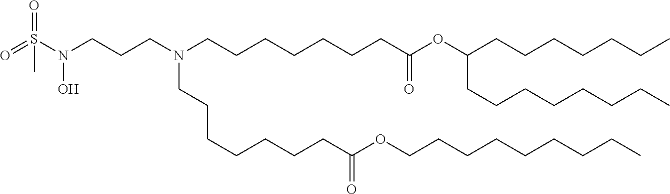 [SMILES: CCCCCCCCCOC(=O)CCCCCCCN(CCCCCCCC(=O)OC(CCCCCCC)CCCCCCCC)CCCN(O)S(C)(=O)=O] |
| I 207 | 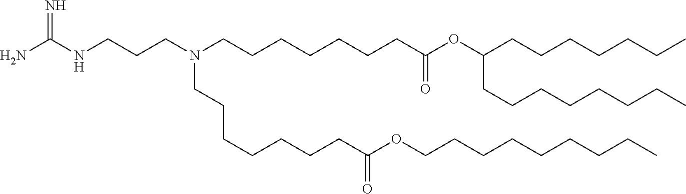 [SMILES: CCCCCCCCCOC(=O)CCCCCCCN(CCCCCCCC(=O)OC(CCCCCCC)CCCCCCCC)CCCNC(=N)N] |
| I 208 | 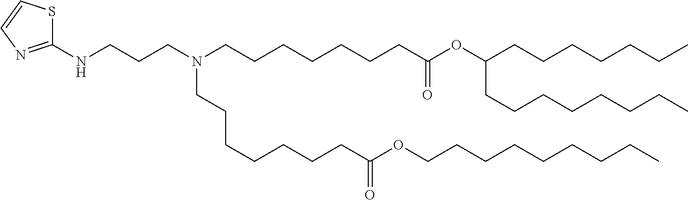 [SMILES: CCCCCCCCCOC(=O)CCCCCCCN(CCCCCCCC(=O)OC(CCCCCCC)CCCCCCCC)CCCNc1nccs1] |
| I 209 | 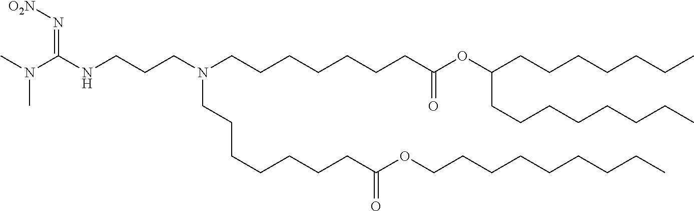 [SMILES: CCCCCCCCCOC(=O)CCCCCCCN(CCCCCCCC(=O)OC(CCCCCCC)CCCCCCCC)CCCN/C(=N/[N+](=O)[O-])N(C)C] |
| I 210 | 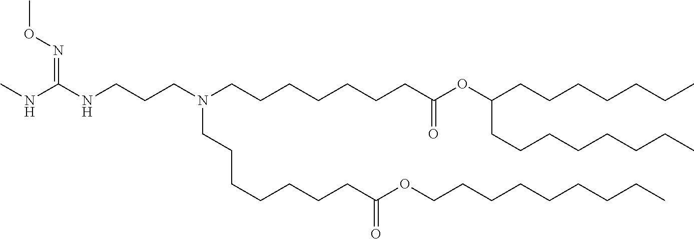 [SMILES: CCCCCCCCCOC(=O)CCCCCCCN(CCCCCCCC(=O)OC(CCCCCCC)CCCCCCCC)CCCN/C(=N/OC)NC] |
| I 211 | 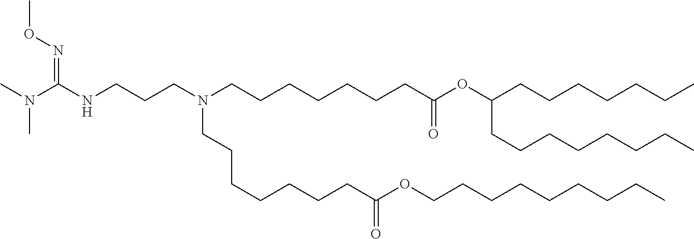 [SMILES: CCCCCCCCCOC(=O)CCCCCCCN(CCCCCCCC(=O)OC(CCCCCCC)CCCCCCCC)CCCN/C(=N/OC)N(C)C] |
| I 212 | 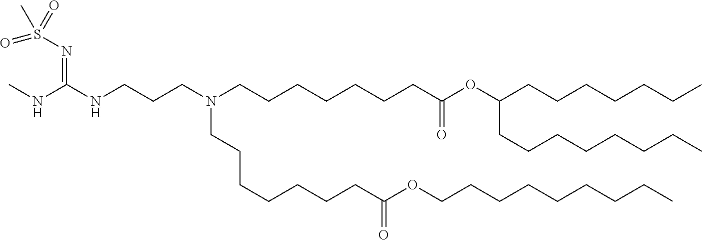 [SMILES: CCCCCCCCCOC(=O)CCCCCCCN(CCCCCCCC(=O)OC(CCCCCCC)CCCCCCCC)CCCN/C(=N/S(C)(=O)=O)NC] |
| I 213 | 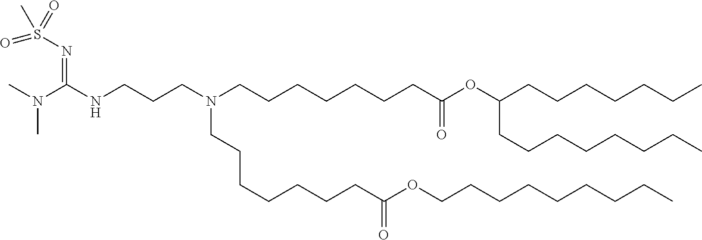 [SMILES: CCCCCCCCCOC(=O)CCCCCCCN(CCCCCCCC(=O)OC(CCCCCCC)CCCCCCCC)CCCN/C(=N/S(C)(=O)=O)N(C)C] |
| I 214 | 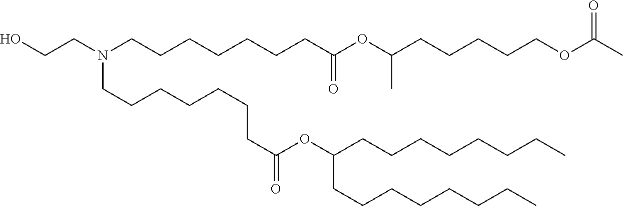 [SMILES: CCCCCCCCC(CCCCCCCC)OC(=O)CCCCCCCN(CCO)CCCCCCCC(=O)OC(C)CCCCCOC(C)=O] |
| I 215 | 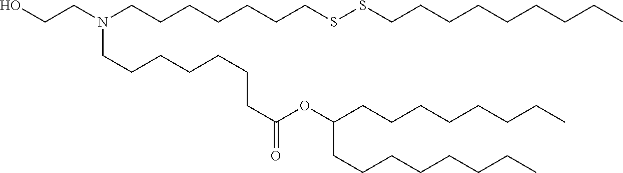 [SMILES: CCCCCCCCCSSCCCCCCCN(CCO)CCCCCCCC(=O)OC(CCCCCCCC)CCCCCCCC] |
| I 216 | 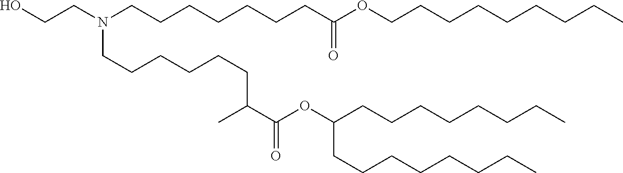 [SMILES: CCCCCCCCCOC(=O)CCCCCCCN(CCO)CCCCCCC(C)C(=O)OC(CCCCCCCC)CCCCCCCC] |
| I 217 | 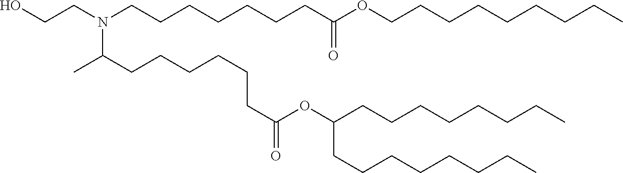 [SMILES: CCCCCCCCCOC(=O)CCCCCCCN(CCO)C(C)CCCCCCC(=O)OC(CCCCCCCC)CCCCCCCC] |
| I 218 | 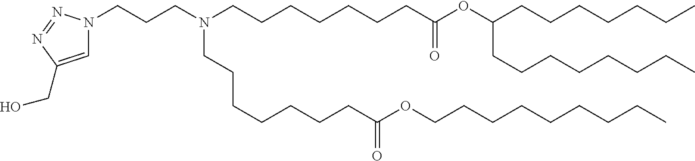 [SMILES: CCCCCCCCCOC(=O)CCCCCCCN(CCCCCCCC(=O)OC(CCCCCCC)CCCCCCCC)CCCn1cc(CO)nn1] |
| I 219 | 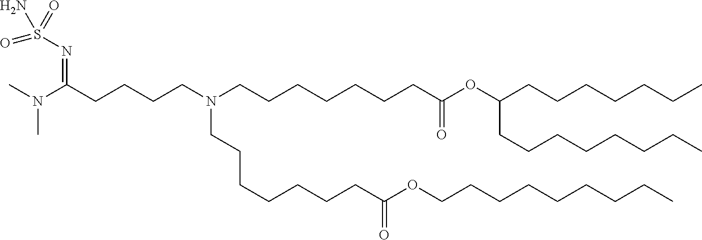 [SMILES: CCCCCCCCCOC(=O)CCCCCCCN(CCCCCCCC(=O)OC(CCCCCCC)CCCCCCCC)CCCC/C(=N/S(N)(=O)=O)N(C)C] |
| I 220 | 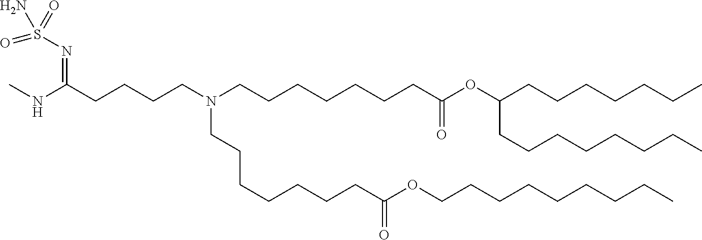 [SMILES: CCCCCCCCCOC(=O)CCCCCCCN(CCCCCCCC(=O)OC(CCCCCCC)CCCCCCCC)CCCC/C(=N/S(N)(=O)=O)NC] |
| I 221 | 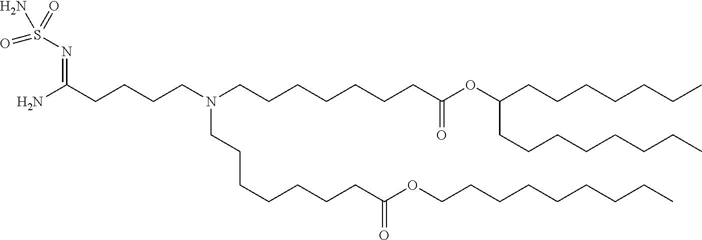 [SMILES: CCCCCCCCCOC(=O)CCCCCCCN(CCCCCCCC(=O)OC(CCCCCCC)CCCCCCCC)CCCC/C(N)=N/S(N)(=O)=O] |
| I 222 | 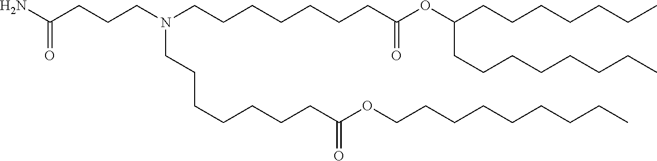 [SMILES: CCCCCCCCCOC(=O)CCCCCCCN(CCCCCCCC(=O)OC(CCCCCCC)CCCCCCCC)CCCC(N)=O] |
| I 223 | 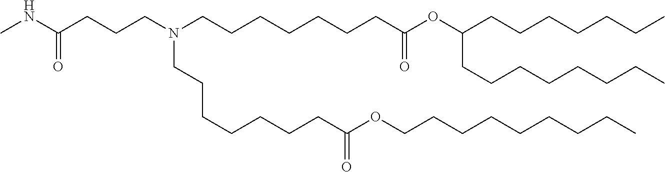 [SMILES: CCCCCCCCCOC(=O)CCCCCCCN(CCCCCCCC(=O)OC(CCCCCCC)CCCCCCCC)CCCC(=O)NC] |
| I 224 | 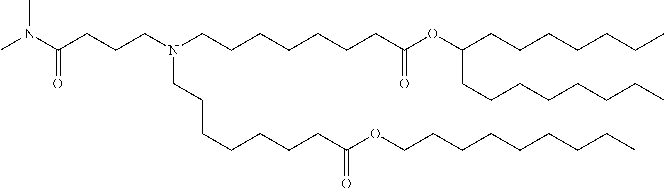 [SMILES: CCCCCCCCCOC(=O)CCCCCCCN(CCCCCCCC(=O)OC(CCCCCCC)CCCCCCCC)CCCC(=O)N(C)C] |
| I 225 | 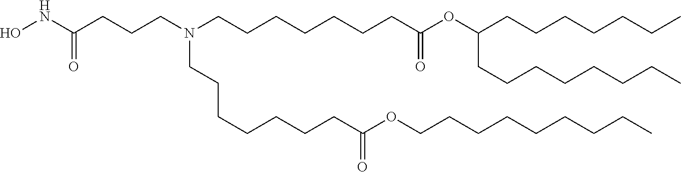 [SMILES: CCCCCCCCCOC(=O)CCCCCCCN(CCCCCCCC(=O)OC(CCCCCCC)CCCCCCCC)CCCC(=O)NO] |
| I 226 | 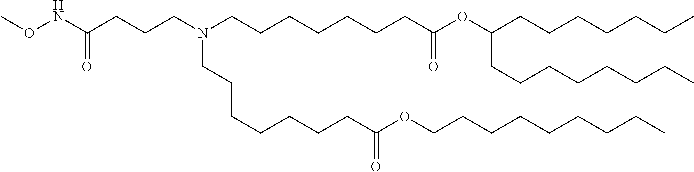 [SMILES: CCCCCCCCCOC(=O)CCCCCCCN(CCCCCCCC(=O)OC(CCCCCCC)CCCCCCCC)CCCC(=O)NOC] |
| I 227 | 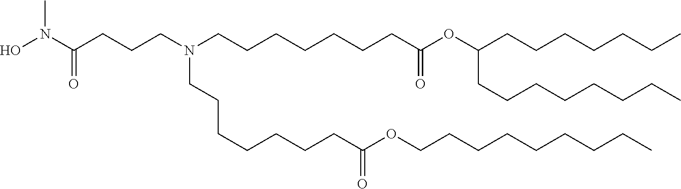 [SMILES: CCCCCCCCCOC(=O)CCCCCCCN(CCCCCCCC(=O)OC(CCCCCCC)CCCCCCCC)CCCC(=O)N(C)O] |
| I 228 | 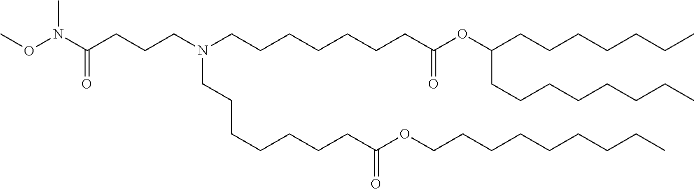 [SMILES: CCCCCCCCCOC(=O)CCCCCCCN(CCCCCCCC(=O)OC(CCCCCCC)CCCCCCCC)CCCC(=O)N(C)OC] |
| I 229 | 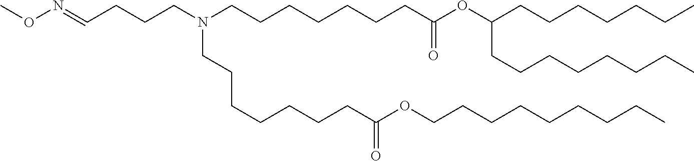 [SMILES: CCCCCCCCCOC(=O)CCCCCCCN(CCC/C=N/OC)CCCCCCCC(=O)OC(CCCCCCC)CCCCCCCC] |
| I 230 | 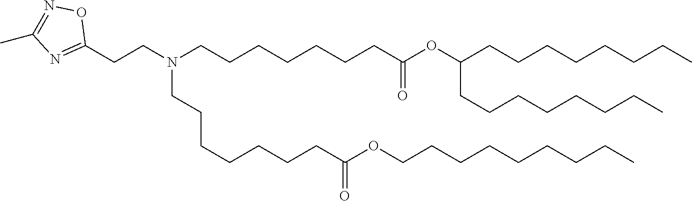 [SMILES: CCCCCCCCCOC(=O)CCCCCCCN(CCCCCCCC(=O)OC(CCCCCCCC)CCCCCCCC)CCc1nc(C)no1] |
| I 231 | 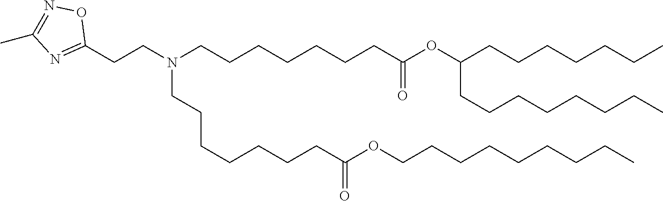 [SMILES: CCCCCCCCCOC(=O)CCCCCCCN(CCCCCCCC(=O)OC(CCCCCCC)CCCCCCCC)CCc1nc(C)no1] |
| I 232 | 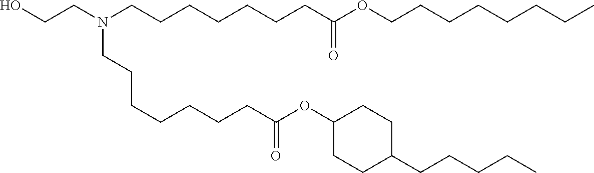 [SMILES: CCCCCCCCOC(=O)CCCCCCCN(CCO)CCCCCCCC(=O)OC1CCC(CCCCC)CC1] |
| I 233 | 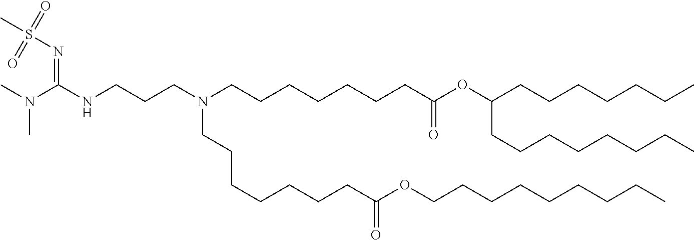 [SMILES: CCCCCCCCCOC(=O)CCCCCCCN(CCCCCCCC(=O)OC(CCCCCCC)CCCCCCCC)CCCN/C(=N/S(C)(=O)=O)N(C)C] |
| I 234 | 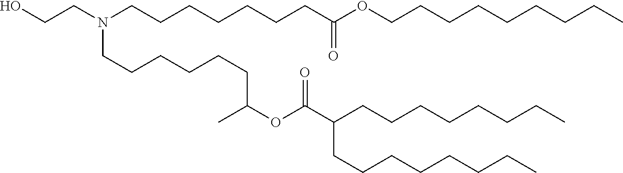 [SMILES: CCCCCCCCCOC(=O)CCCCCCCN(CCO)CCCCCCC(C)OC(=O)C(CCCCCCCC)CCCCCCCC] |
| I 235 | 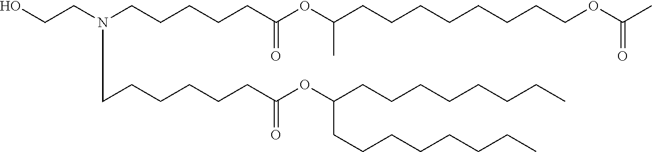 [SMILES: CCCCCCCCC(CCCCCCCC)OC(=O)CCCCCCN(CCO)CCCCCC(=O)OC(C)CCCCCCCCOC(C)=O] |
| I 236 | 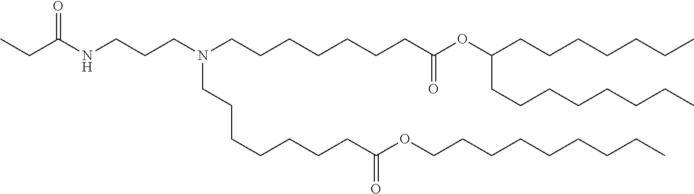 [SMILES: CCCCCCCCCOC(=O)CCCCCCCN(CCCCCCCC(=O)OC(CCCCCCC)CCCCCCCC)CCCNC(=O)CC] |
| I 237 | 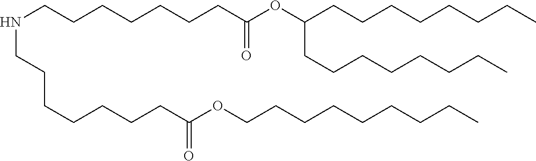 [SMILES: CCCCCCCCCOC(=O)CCCCCCCNCCCCCCCC(=O)OC(CCCCCCCC)CCCCCCCC] |
| I 238 | 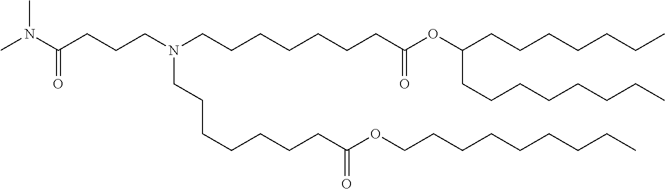 [SMILES: CCCCCCCCCOC(=O)CCCCCCCN(CCCCCCCC(=O)OC(CCCCCCC)CCCCCCCC)CCCC(=O)N(C)C] |
| I 239 | 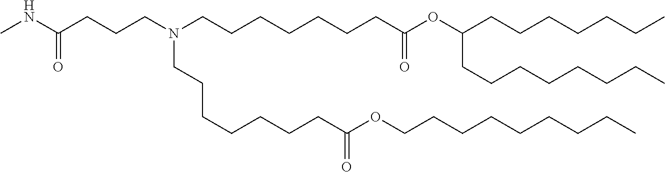 [SMILES: CCCCCCCCCOC(=O)CCCCCCCN(CCCCCCCC(=O)OC(CCCCCCC)CCCCCCCC)CCCC(=O)NC] |
| I 240 | 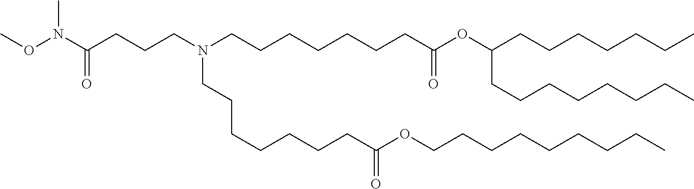 [SMILES: CCCCCCCCCOC(=O)CCCCCCCN(CCCCCCCC(=O)OC(CCCCCCC)CCCCCCCC)CCCC(=O)N(C)OC] |
| I 241 | 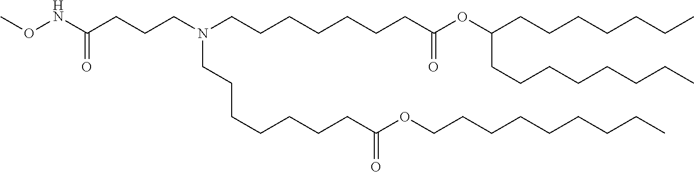 [SMILES: CCCCCCCCCOC(=O)CCCCCCCN(CCCCCCCC(=O)OC(CCCCCCC)CCCCCCCC)CCCC(=O)NOC] |
| I 242 | 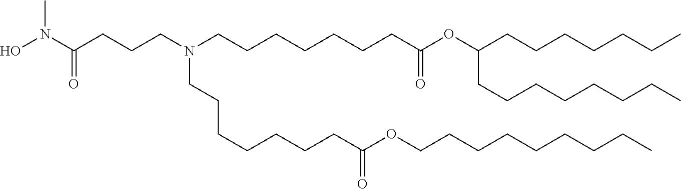 [SMILES: CCCCCCCCCOC(=O)CCCCCCCN(CCCCCCCC(=O)OC(CCCCCCC)CCCCCCCC)CCCC(=O)N(C)O] |
| I 243 | 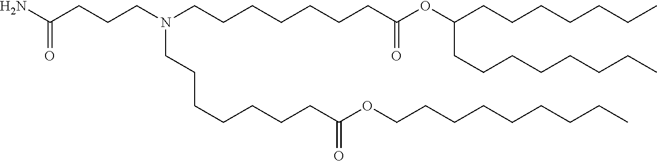 [SMILES: CCCCCCCCCOC(=O)CCCCCCCN(CCCCCCCC(=O)OC(CCCCCCC)CCCCCCCC)CCCC(N)=O] |
| I 244 | 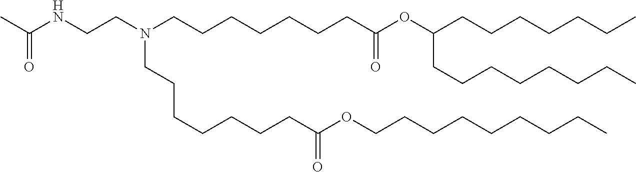 [SMILES: CCCCCCCCCOC(=O)CCCCCCCN(CCCCCCCC(=O)OC(CCCCCCC)CCCCCCCC)CCNC(C)=O] |
| I 245 | 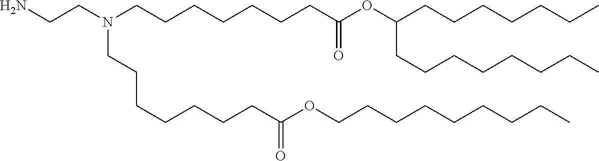 [SMILES: CCCCCCCCCOC(=O)CCCCCCCN(CCN)CCCCCCCC(=O)OC(CCCCCCC)CCCCCCCC] |
| I 246 | 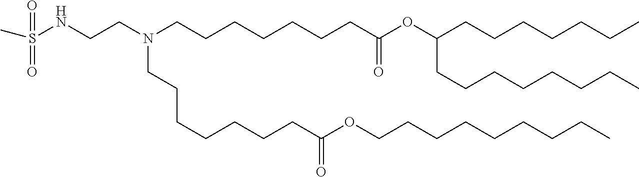 [SMILES: CCCCCCCCCOC(=O)CCCCCCCN(CCCCCCCC(=O)OC(CCCCCCC)CCCCCCCC)CCNS(C)(=O)=O] |
| I 247 | 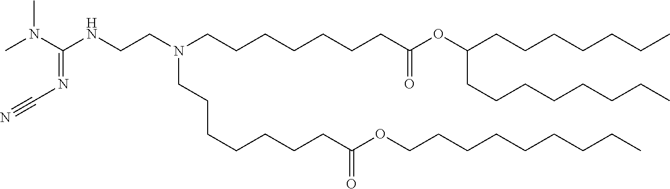 [SMILES: CCCCCCCCCOC(=O)CCCCCCCN(CCCCCCCC(=O)OC(CCCCCCC)CCCCCCCC)CCN/C(=N/C#N)N(C)C] |
| I 248 | 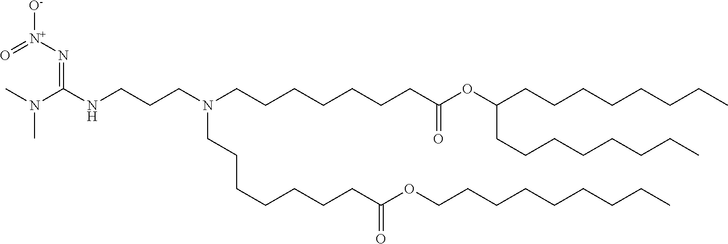 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I 249 | 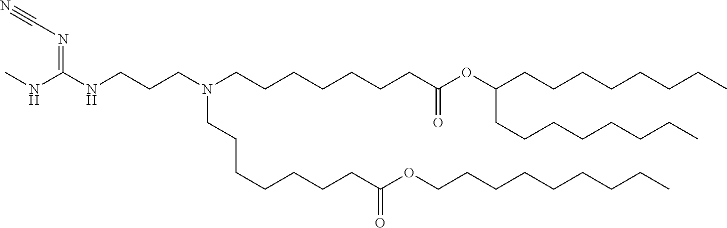 [SMILES: CCCCCCCCCOC(=O)CCCCCCCN(CCCCCCCC(=O)OC(CCCCCCCC)CCCCCCCC)CCCN/C(=N/C#N)NC] |
| I 250 | 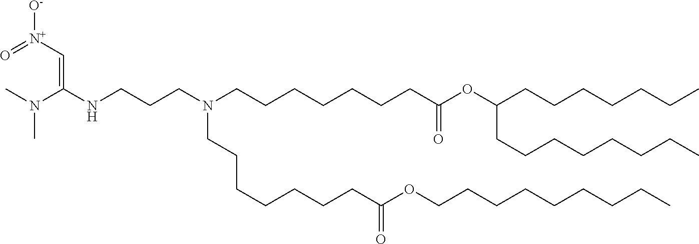 [SMILES: CCCCCCCCCOC(=O)CCCCCCCN(CCCCCCCC(=O)OC(CCCCCCC)CCCCCCCC)CCCN/C(=C/[N+](=O)[O-])N(C)C] |
| I 251 | 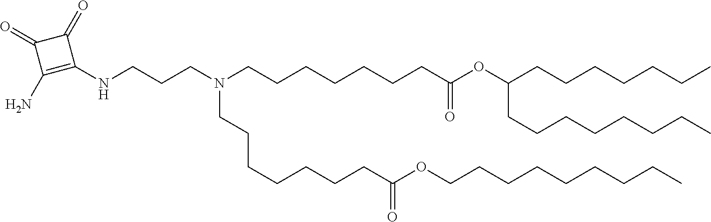 [SMILES: CCCCCCCCCOC(=O)CCCCCCCN(CCCCCCCC(=O)OC(CCCCCCC)CCCCCCCC)CCCNc1c(N)c(=O)c1=O] |
| I 252 | 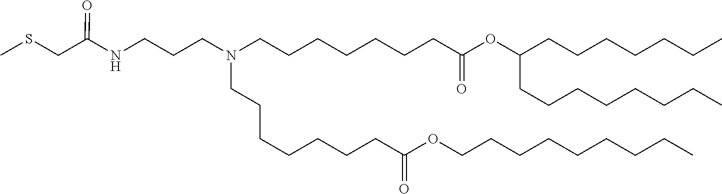 [SMILES: CCCCCCCCCOC(=O)CCCCCCCN(CCCCCCCC(=O)OC(CCCCCCC)CCCCCCCC)CCCNC(=O)CSC] |
| I 253 | 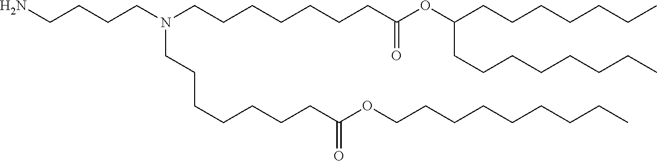 [SMILES: CCCCCCCCCOC(=O)CCCCCCCN(CCCCN)CCCCCCCC(=O)OC(CCCCCCC)CCCCCCCC] |
| I 254 | 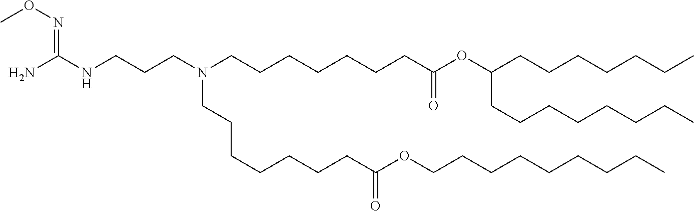 [SMILES: CCCCCCCCCOC(=O)CCCCCCCN(CCCCCCCC(=O)OC(CCCCCCC)CCCCCCCC)CCCN/C(N)=N/OC] |
| I 255 | 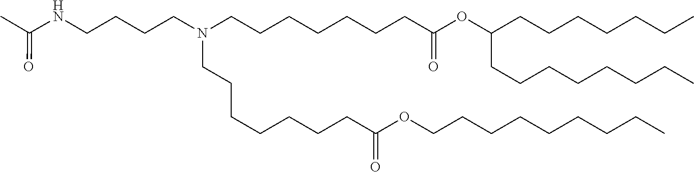 [SMILES: CCCCCCCCCOC(=O)CCCCCCCN(CCCCCCCC(=O)OC(CCCCCCC)CCCCCCCC)CCCCNC(C)=O] |
| I 256 | 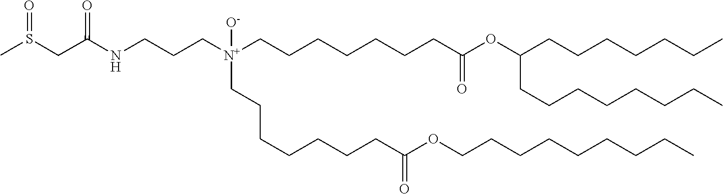 [SMILES: CCCCCCCCCOC(=O)CCCCCCC[N+]([O-])(CCCCCCCC(=O)OC(CCCCCCC)CCCCCCCC)CCCNC(=O)CS(C)=O] |
| I 257 | 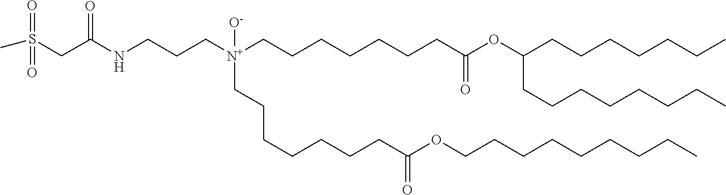 [SMILES: CCCCCCCCCOC(=O)CCCCCCC[N+]([O-])(CCCCCCCC(=O)OC(CCCCCCC)CCCCCCCC)CCCNC(=O)CS(C)(=O)=O] |
| I 258 | 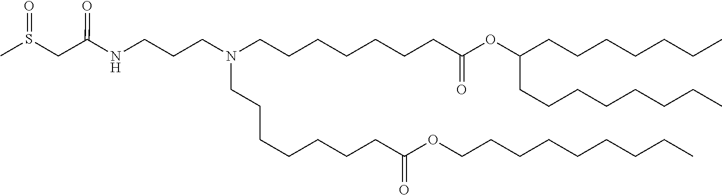 [SMILES: CCCCCCCCCOC(=O)CCCCCCCN(CCCCCCCC(=O)OC(CCCCCCC)CCCCCCCC)CCCNC(=O)CS(C)=O] |
| I 259 | 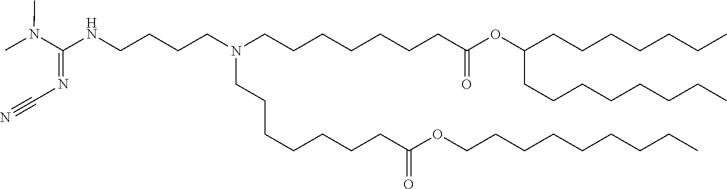 [SMILES: CCCCCCCCCOC(=O)CCCCCCCN(CCCCCCCC(=O)OC(CCCCCCC)CCCCCCCC)CCCCN/C(=N/C#N)N(C)C] |
| I 260 | 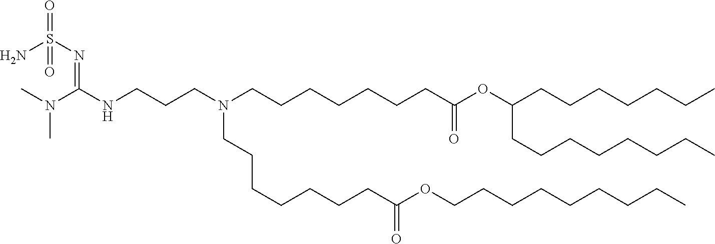 [SMILES: CCCCCCCCCOC(=O)CCCCCCCN(CCCCCCCC(=O)OC(CCCCCCC)CCCCCCCC)CCCN/C(=N/S(N)(=O)=O)N(C)C] |
| I 261 | 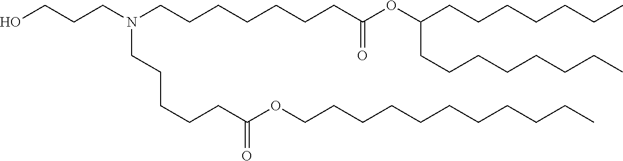 [SMILES: CCCCCCCCCCCOC(=O)CCCCCN(CCCO)CCCCCCCC(=O)OC(CCCCCCC)CCCCCCCC] |
| I 262 | 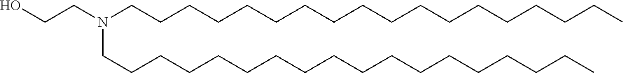 [SMILES: CCCCCCCCCCCCCCCCCCN(CCO)CCCCCCCCCCCCCCCCCC] |
| I 263 | 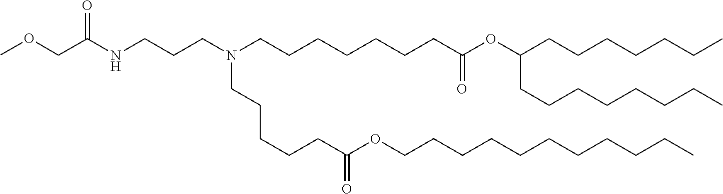 [SMILES: CCCCCCCCCCCOC(=O)CCCCCN(CCCCCCCC(=O)OC(CCCCCCC)CCCCCCCC)CCCNC(=O)COC] |
| I 264 | 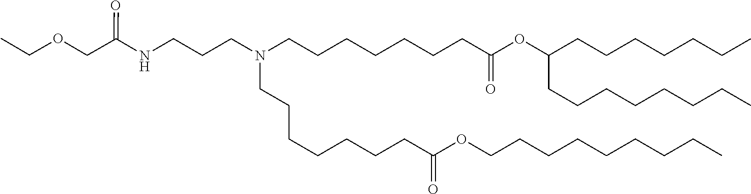 [SMILES: CCCCCCCCCOC(=O)CCCCCCCN(CCCCCCCC(=O)OC(CCCCCCC)CCCCCCCC)CCCNC(=O)COCC] |
| I 265 | 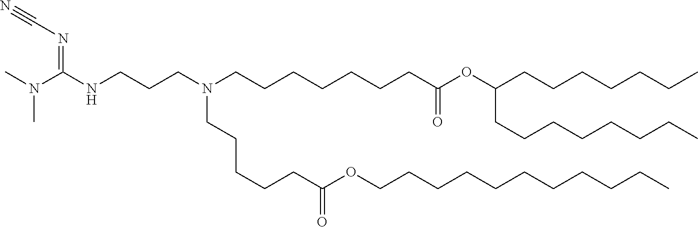 [SMILES: CCCCCCCCCCCOC(=O)CCCCCN(CCCCCCCC(=O)OC(CCCCCCC)CCCCCCCC)CCCN/C(=N/C#N)N(C)C] |
| I 266 | 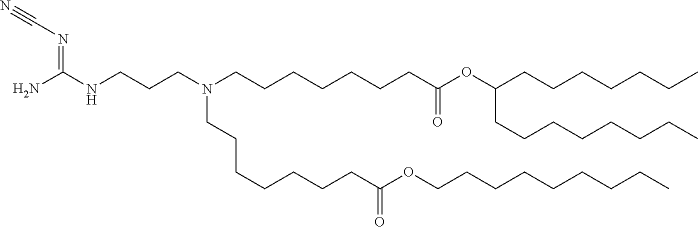 [SMILES: CCCCCCCCCOC(=O)CCCCCCCN(CCCCCCCC(=O)OC(CCCCCCC)CCCCCCCC)CCCN/C(N)=N/C#N] |
| I 267 | 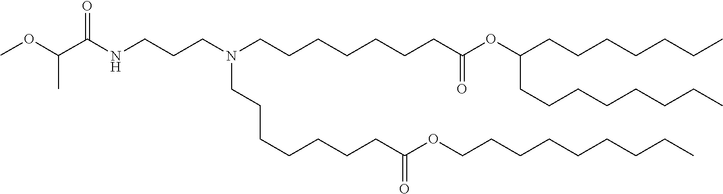 [SMILES: CCCCCCCCCOC(=O)CCCCCCCN(CCCCCCCC(=O)OC(CCCCCCC)CCCCCCCC)CCCNC(=O)C(C)OC] |
| I 268 | 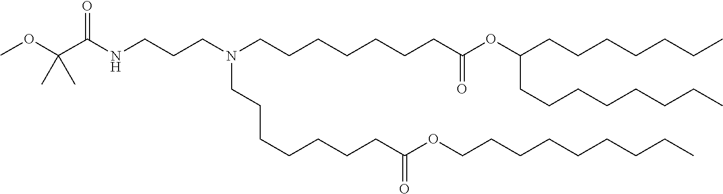 [SMILES: CCCCCCCCCOC(=O)CCCCCCCN(CCCCCCCC(=O)OC(CCCCCCC)CCCCCCCC)CCCNC(=O)C(C)(C)OC] |
| I 269 | 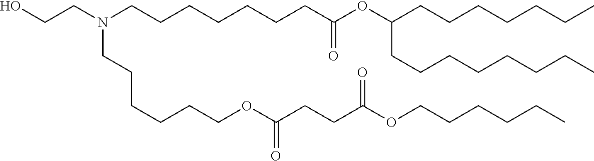 [SMILES: CCCCCCCCC(CCCCCCC)OC(=O)CCCCCCCN(CCO)CCCCCCOC(=O)CCC(=O)OCCCCCC] |
| I 270 | 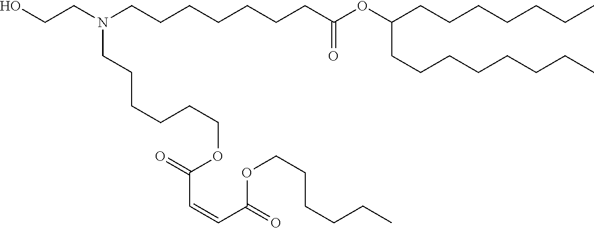 [SMILES: CCCCCCCCC(CCCCCCC)OC(=O)CCCCCCCN(CCO)CCCCCCOC(=O)/C=C\C(=O)OCCCCCC] |
| I 271 | 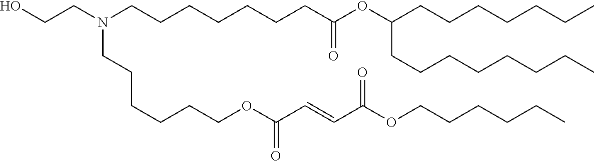 [SMILES: CCCCCCCCC(CCCCCCC)OC(=O)CCCCCCCN(CCO)CCCCCCOC(=O)/C=C/C(=O)OCCCCCC] |
| I 272 | 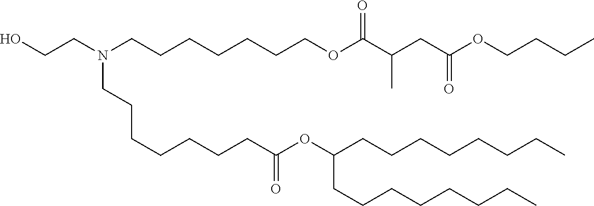 [SMILES: CCCCCCCCC(CCCCCCCC)OC(=O)CCCCCCCN(CCO)CCCCCCCOC(=O)C(C)CC(=O)OCCCC] |
| I 273 | 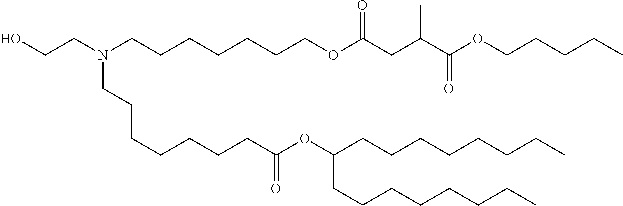 [SMILES: CCCCCCCCC(CCCCCCCC)OC(=O)CCCCCCCN(CCO)CCCCCCCOC(=O)CC(C)C(=O)OCCCCC] |
| I 274 | 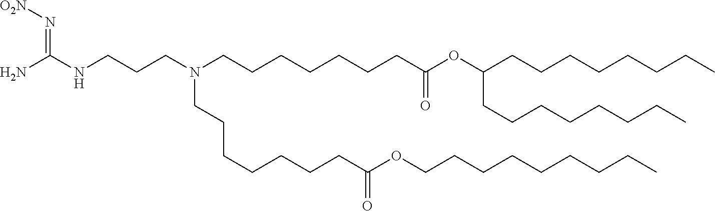 [SMILES: CCCCCCCCCOC(=O)CCCCCCCN(CCCCCCCC(=O)OC(CCCCCCCC)CCCCCCCC)CCCN/C(N)=N/[N+](=O)[O-]] |
| I 275 | 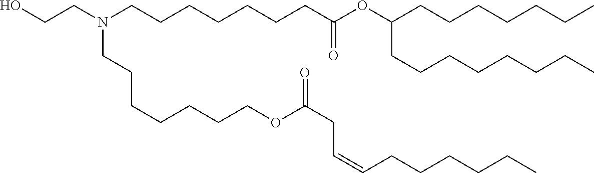 [SMILES: CCCCCC/C=C\CC(=O)OCCCCCCCN(CCO)CCCCCCCC(=O)OC(CCCCCCC)CCCCCCCC] |
| I 276 | 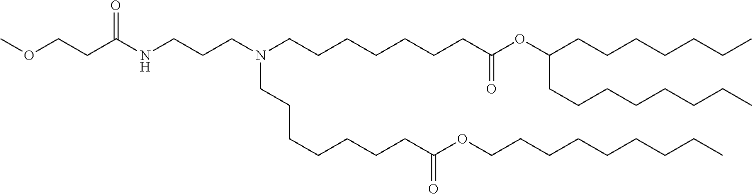 [SMILES: CCCCCCCCCOC(=O)CCCCCCCN(CCCCCCCC(=O)OC(CCCCCCC)CCCCCCCC)CCCNC(=O)CCOC] |
| I 277 | 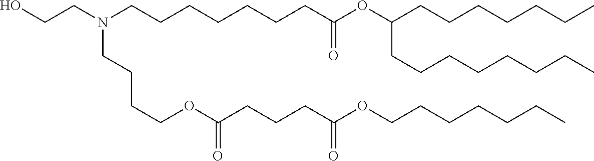 [SMILES: CCCCCCCCC(CCCCCCC)OC(=O)CCCCCCCN(CCO)CCCCOC(=O)CCCC(=O)OCCCCCCC] |
| I 278 | 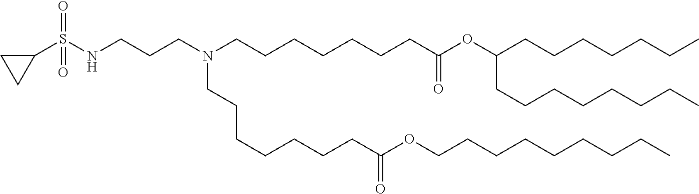 [SMILES: CCCCCCCCCOC(=O)CCCCCCCN(CCCCCCCC(=O)OC(CCCCCCC)CCCCCCCC)CCCNS(=O)(=O)C1CC1] |
| I 279 | 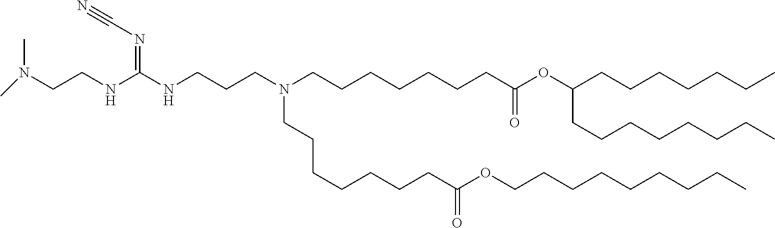 [SMILES: CCCCCCCCCOC(=O)CCCCCCCN(CCCCCCCC(=O)OC(CCCCCCC)CCCCCCCC)CCCN/C(=N/C#N)NCCN(C)C] |
| I 280 | 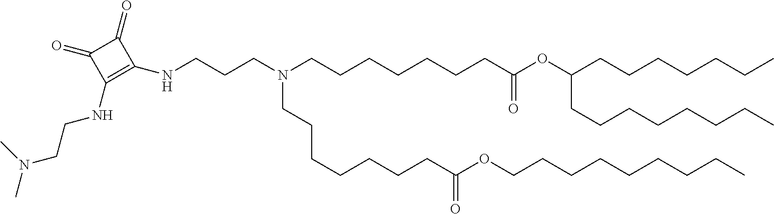 [SMILES: CCCCCCCCCOC(=O)CCCCCCCN(CCCCCCCC(=O)OC(CCCCCCC)CCCCCCCC)CCCNc1c(NCCN(C)C)c(=O)c1=O] |
| I 281 | 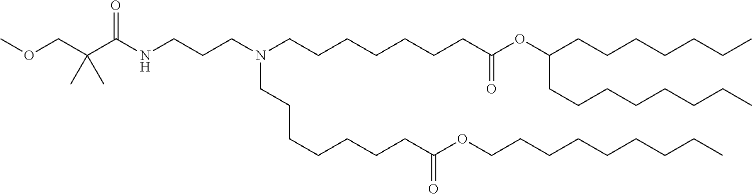 [SMILES: CCCCCCCCCOC(=O)CCCCCCCN(CCCCCCCC(=O)OC(CCCCCCC)CCCCCCCC)CCCNC(=O)C(C)(C)COC] |
| I 282 | 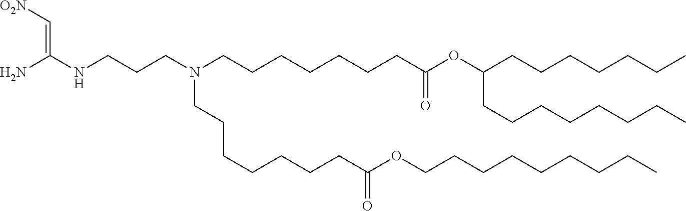 [SMILES: CCCCCCCCCOC(=O)CCCCCCCN(CCCCCCCC(=O)OC(CCCCCCC)CCCCCCCC)CCCN/C(N)=C/[N+](=O)[O-]] |
| I 283 | 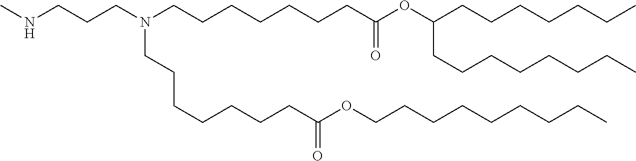 [SMILES: CCCCCCCCCOC(=O)CCCCCCCN(CCCCCCCC(=O)OC(CCCCCCC)CCCCCCCC)CCCNC] |
| I 284 | 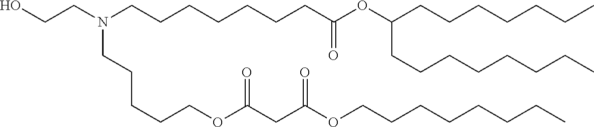 [SMILES: CCCCCCCCOC(=O)CC(=O)OCCCCCN(CCO)CCCCCCCC(=O)OC(CCCCCCC)CCCCCCCC] |
| I 285 | 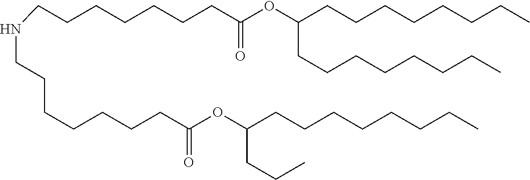 [SMILES: CCCCCCCCC(CCC)OC(=O)CCCCCCCNCCCCCCCC(=O)OC(CCCCCCCC)CCCCCCCC] |
| I 286 | 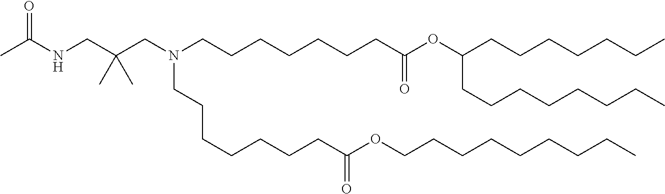 [SMILES: CCCCCCCCCOC(=O)CCCCCCCN(CCCCCCCC(=O)OC(CCCCCCC)CCCCCCCC)CC(C)(C)CNC(C)=O] |
| I 287 | 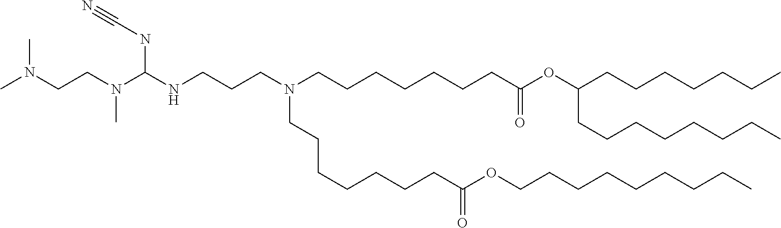 [SMILES: CCCCCCCCCOC(=O)CCCCCCCN(CCCCCCCC(=O)OC(CCCCCCC)CCCCCCCC)CCCNC(NC#N)N(C)CCN(C)C] |
| I 288 | 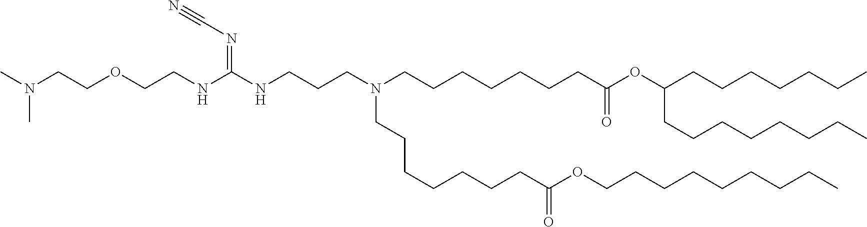 [SMILES: CCCCCCCCCOC(=O)CCCCCCCN(CCCCCCCC(=O)OC(CCCCCCC)CCCCCCCC)CCCN/C(=N/C#N)NCCOCCN(C)C] |
| I 289 | 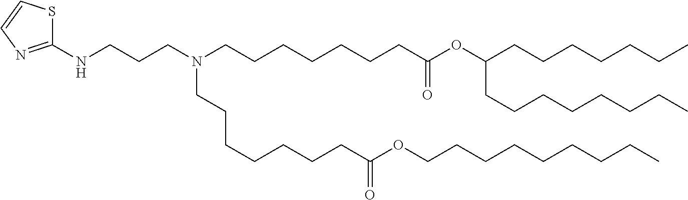 [SMILES: CCCCCCCCCOC(=O)CCCCCCCN(CCCCCCCC(=O)OC(CCCCCCC)CCCCCCCC)CCCNc1nccs1] |
| I 290 | 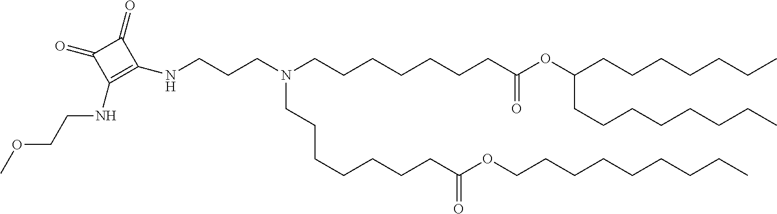 [SMILES: CCCCCCCCCOC(=O)CCCCCCCN(CCCCCCCC(=O)OC(CCCCCCC)CCCCCCCC)CCCNc1c(NCCOC)c(=O)c1=O] |
| I 291 | 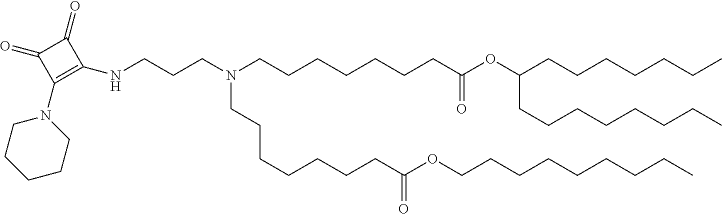 [SMILES: CCCCCCCCCOC(=O)CCCCCCCN(CCCCCCCC(=O)OC(CCCCCCC)CCCCCCCC)CCCNc1c(N2CCCCC2)c(=O)c1=O] |
| I 292 | 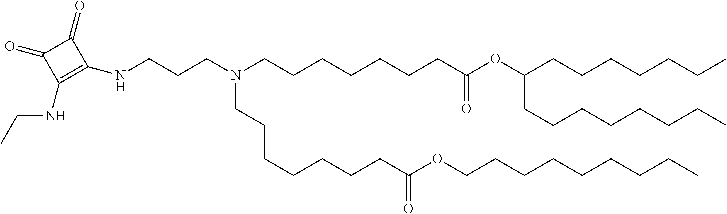 [SMILES: CCCCCCCCCOC(=O)CCCCCCCN(CCCCCCCC(=O)OC(CCCCCCC)CCCCCCCC)CCCNc1c(NCC)c(=O)c1=O] |
| I 293 | 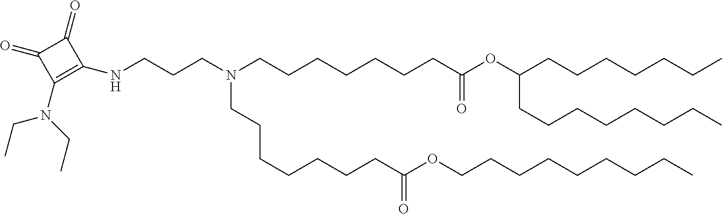 [SMILES: CCCCCCCCCOC(=O)CCCCCCCN(CCCCCCCC(=O)OC(CCCCCCC)CCCCCCCC)CCCNc1c(N(CC)CC)c(=O)c1=O] |
| I 294 | 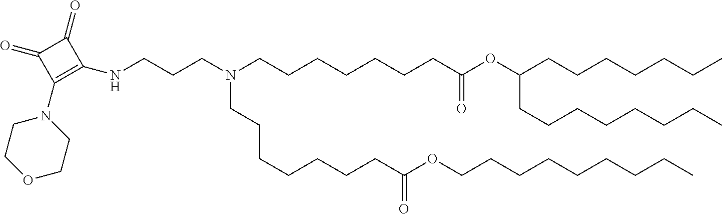 [SMILES: CCCCCCCCCOC(=O)CCCCCCCN(CCCCCCCC(=O)OC(CCCCCCC)CCCCCCCC)CCCNc1c(N2CCOCC2)c(=O)c1=O] |
| I 295 | 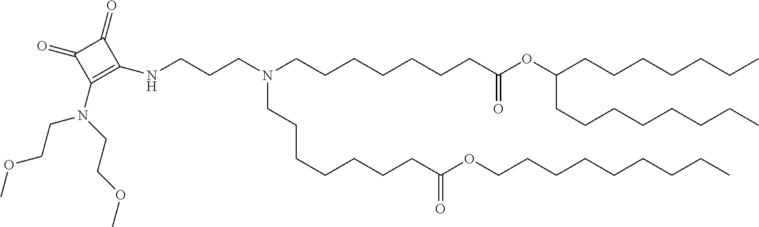 [SMILES: CCCCCCCCCOC(=O)CCCCCCCN(CCCCCCCC(=O)OC(CCCCCCC)CCCCCCCC)CCCNc1c(N(CCOC)CCOC)c(=O)c1=O] |
| I 296 | 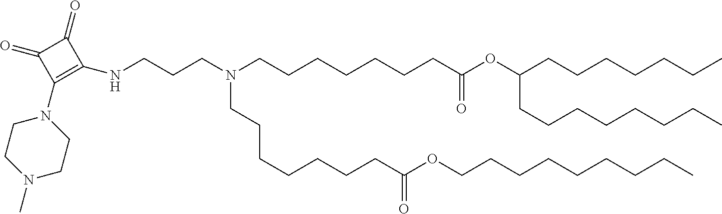 [SMILES: CCCCCCCCCOC(=O)CCCCCCCN(CCCCCCCC(=O)OC(CCCCCCC)CCCCCCCC)CCCNc1c(N2CCN(C)CC2)c(=O)c1=O] |
| I 297 | 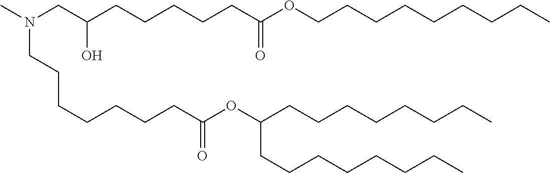 [SMILES: CCCCCCCCCOC(=O)CCCCCC(O)CN(C)CCCCCCCC(=O)OC(CCCCCCCC)CCCCCCCC] |
| I 298 | 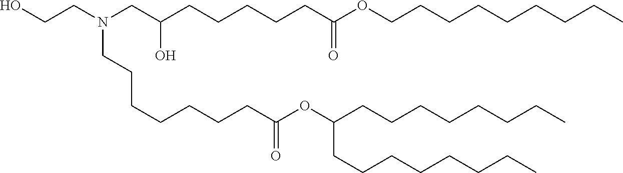 [SMILES: CCCCCCCCCOC(=O)CCCCCC(O)CN(CCO)CCCCCCCC(=O)OC(CCCCCCCC)CCCCCCCC] |
| I 299 | 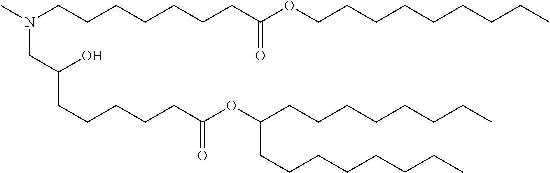 [SMILES: CCCCCCCCCOC(=O)CCCCCCCN(C)CC(O)CCCCCC(=O)OC(CCCCCCCC)CCCCCCCC] |
| I 300 | 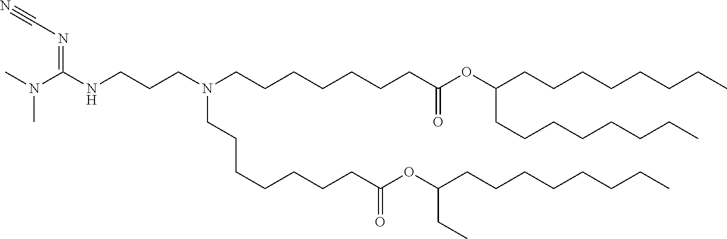 [SMILES: CCCCCCCCC(CC)OC(=O)CCCCCCCN(CCCCCCCC(=O)OC(CCCCCCCC)CCCCCCCC)CCCN/C(=N/C#N)N(C)C] |
| I 301 | 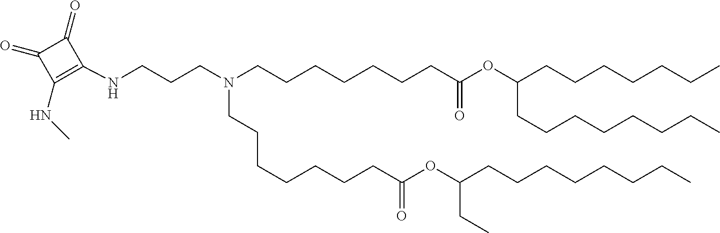 [SMILES: CCCCCCCCC(CC)OC(=O)CCCCCCCN(CCCCCCCC(=O)OC(CCCCCCC)CCCCCCCC)CCCNc1c(NC)c(=O)c1=O] |
| I 302 | 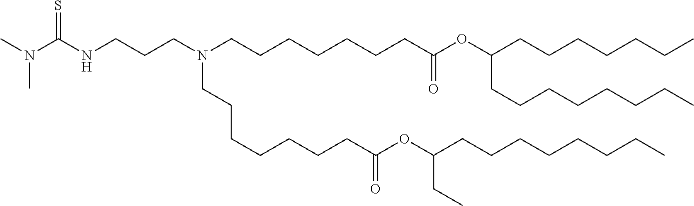 [SMILES: CCCCCCCCC(CC)OC(=O)CCCCCCCN(CCCCCCCC(=O)OC(CCCCCCC)CCCCCCCC)CCCNC(=S)N(C)C] |
| I 303 | 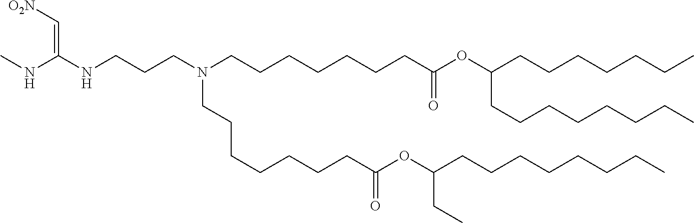 [SMILES: CCCCCCCCC(CC)OC(=O)CCCCCCCN(CCCCCCCC(=O)OC(CCCCCCC)CCCCCCCC)CCCN/C(=C/[N+](=O)[O-])NC] |
| I 304 | 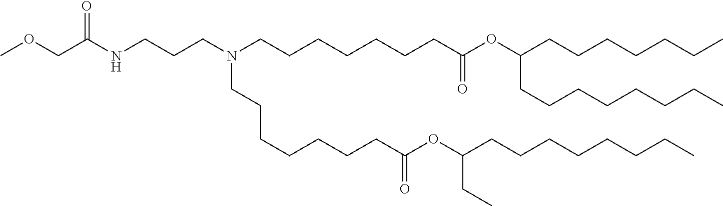 [SMILES: CCCCCCCCC(CC)OC(=O)CCCCCCCN(CCCCCCCC(=O)OC(CCCCCCC)CCCCCCCC)CCCNC(=O)COC] |
| I 305 | 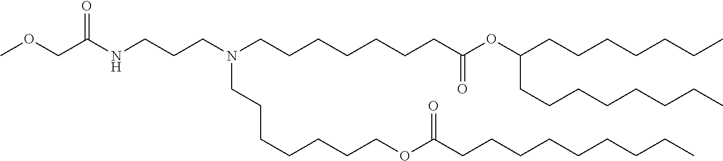 [SMILES: CCCCCCCCCC(=O)OCCCCCCCN(CCCCCCCC(=O)OC(CCCCCCC)CCCCCCCC)CCCNC(=O)COC] |
| I 306 | 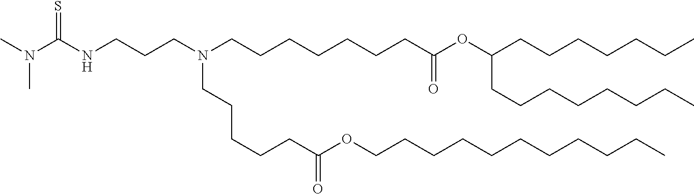 [SMILES: CCCCCCCCCCCOC(=O)CCCCCN(CCCCCCCC(=O)OC(CCCCCCC)CCCCCCCC)CCCNC(=S)N(C)C] |
| I 307 | 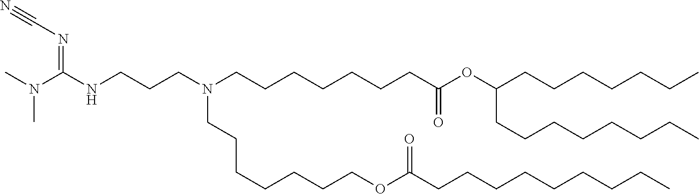 [SMILES: CCCCCCCCCC(=O)OCCCCCCCN(CCCCCCCC(=O)OC(CCCCCCC)CCCCCCCC)CCCN/C(=N/C#N)N(C)C] |
| I 308 | 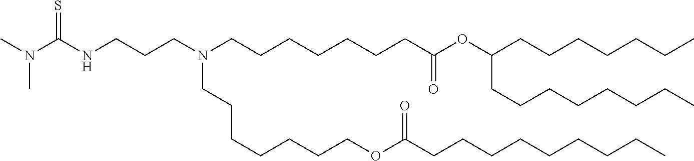 [SMILES: CCCCCCCCCC(=O)OCCCCCCCN(CCCCCCCC(=O)OC(CCCCCCC)CCCCCCCC)CCCNC(=S)N(C)C] |
| I 309 | 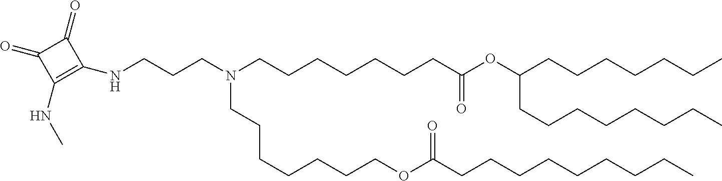 [SMILES: CCCCCCCCCC(=O)OCCCCCCCN(CCCCCCCC(=O)OC(CCCCCCC)CCCCCCCC)CCCNc1c(NC)c(=O)c1=O] |
| I 310 | 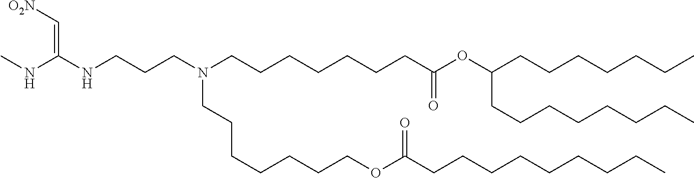 [SMILES: CCCCCCCCCC(=O)OCCCCCCCN(CCCCCCCC(=O)OC(CCCCCCC)CCCCCCCC)CCCN/C(=C/[N+](=O)[O-])NC] |
| I 311 | 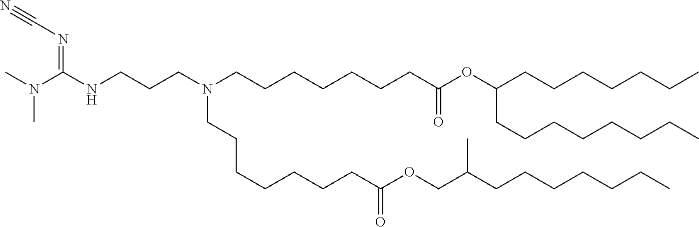 [SMILES: CCCCCCCCC(CCCCCCC)OC(=O)CCCCCCCN(CCCCCCCC(=O)OCC(C)CCCCCCC)CCCN/C(=N/C#N)N(C)C] |
| I 312 | 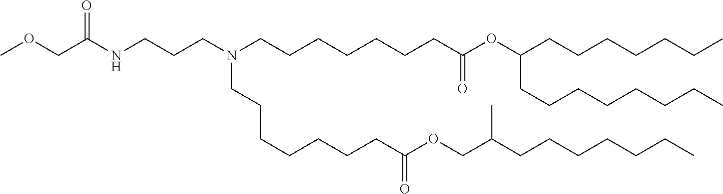 [SMILES: CCCCCCCCC(CCCCCCC)OC(=O)CCCCCCCN(CCCCCCCC(=O)OCC(C)CCCCCCC)CCCNC(=O)COC] |
| I 313 | 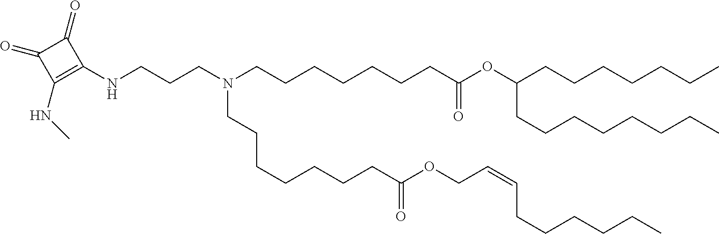 [SMILES: CCCCCC/C=C\COC(=O)CCCCCCCN(CCCCCCCC(=O)OC(CCCCCCC)CCCCCCCC)CCCNc1c(NC)c(=O)c1=O] |
| I 314 | 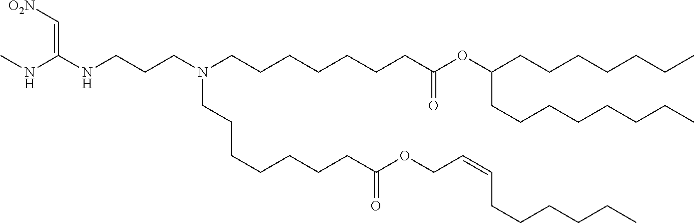 [SMILES: CCCCCC/C=C\COC(=O)CCCCCCCN(CCCCCCCC(=O)OC(CCCCCCC)CCCCCCCC)CCCN/C(=C/[N+](=O)[O-])NC] |
| I 315 | 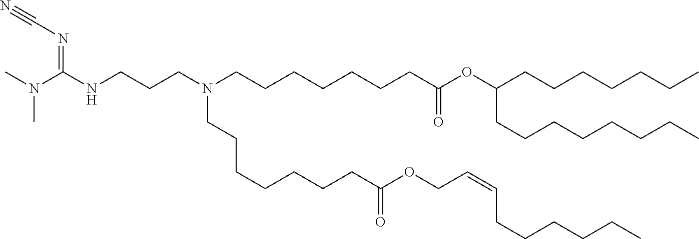 [SMILES: CCCCCC/C=C\COC(=O)CCCCCCCN(CCCCCCCC(=O)OC(CCCCCCC)CCCCCCCC)CCCN/C(=N/C#N)N(C)C] |
| I 316 | 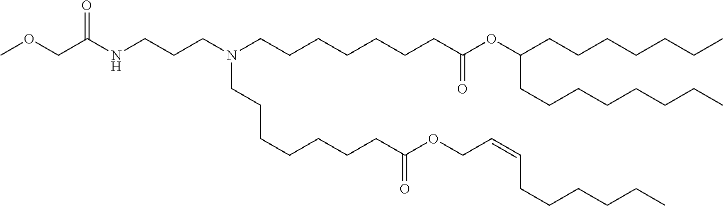 [SMILES: CCCCCC/C=C\COC(=O)CCCCCCCN(CCCCCCCC(=O)OC(CCCCCCC)CCCCCCCC)CCCNC(=O)COC] |
| I 317 | 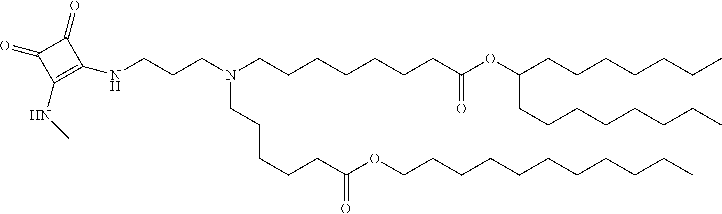 [SMILES: CCCCCCCCCCCOC(=O)CCCCCN(CCCCCCCC(=O)OC(CCCCCCC)CCCCCCCC)CCCNc1c(NC)c(=O)c1=O] |
| I 318 | 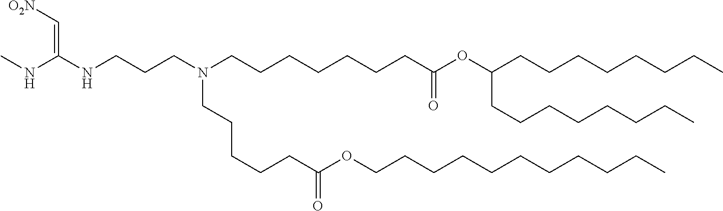 [SMILES: CCCCCCCCCCCOC(=O)CCCCCN(CCCCCCCC(=O)OC(CCCCCCCC)CCCCCCCC)CCCN/C(=C/[N+](=O)[O-])NC] |
| I 319 | 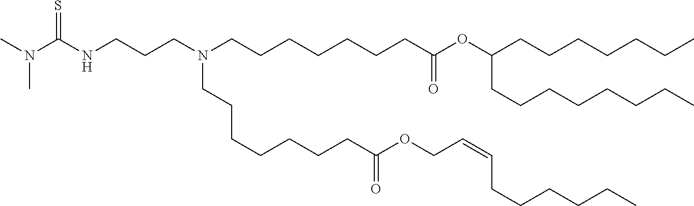 [SMILES: CCCCCC/C=C\COC(=O)CCCCCCCN(CCCCCCCC(=O)OC(CCCCCCC)CCCCCCCC)CCCNC(=S)N(C)C] |
| I 320 | 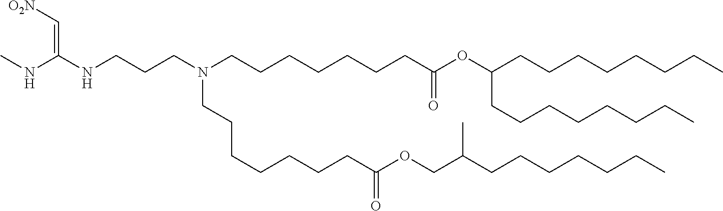 [SMILES: CCCCCCCCC(CCCCCCCC)OC(=O)CCCCCCCN(CCCCCCCC(=O)OCC(C)CCCCCCC)CCCN/C(=C/[N+](=O)[O-])NC] |
| I 321 | 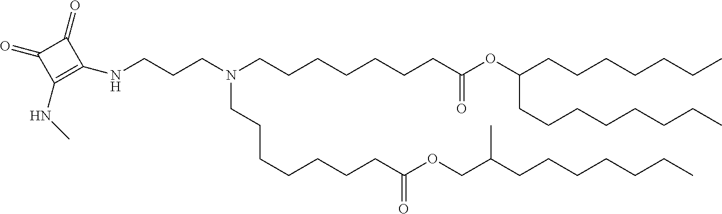 [SMILES: CCCCCCCCC(CCCCCCC)OC(=O)CCCCCCCN(CCCCCCCC(=O)OCC(C)CCCCCCC)CCCNc1c(NC)c(=O)c1=O] |
| I 322 | 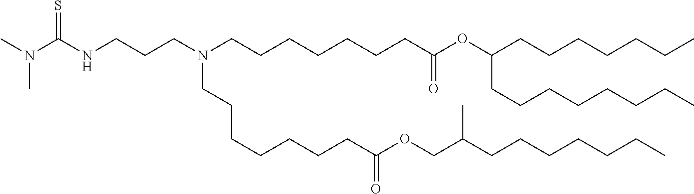 [SMILES: CCCCCCCCC(CCCCCCC)OC(=O)CCCCCCCN(CCCCCCCC(=O)OCC(C)CCCCCCC)CCCNC(=S)N(C)C] |
| I 323 | 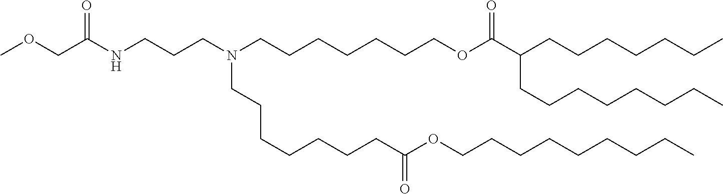 [SMILES: CCCCCCCCCOC(=O)CCCCCCCN(CCCCCCCOC(=O)C(CCCCCCC)CCCCCCCC)CCCNC(=O)COC] |
| I 324 | 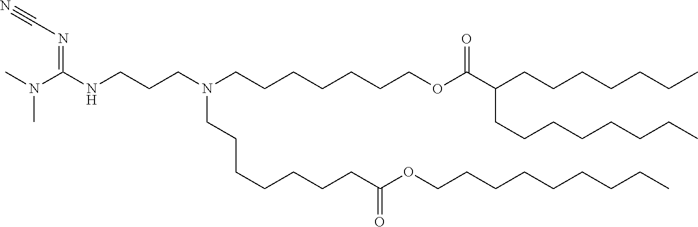 [SMILES: CCCCCCCCCOC(=O)CCCCCCCN(CCCCCCCOC(=O)C(CCCCCCC)CCCCCCCC)CCCN/C(=N/C#N)N(C)C] |
| I 325 | 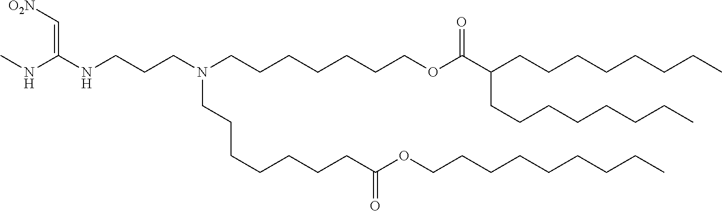 [SMILES: CCCCCCCCCOC(=O)CCCCCCCN(CCCCCCCOC(=O)C(CCCCCCCC)CCCCCCCC)CCCN/C(=C/[N+](=O)[O-])NC] |
| I 326 | 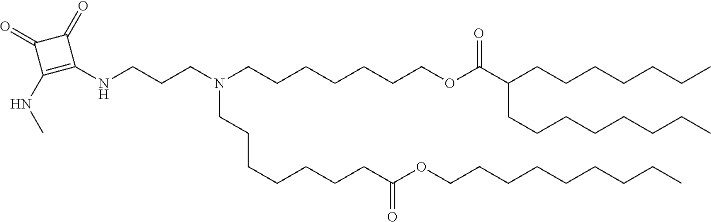 [SMILES: CCCCCCCCCOC(=O)CCCCCCCN(CCCCCCCOC(=O)C(CCCCCCC)CCCCCCCC)CCCNc1c(NC)c(=O)c1=O] |
| I 327 | 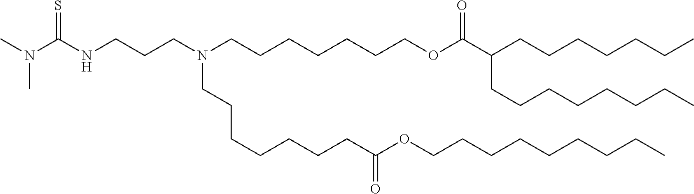 [SMILES: CCCCCCCCCOC(=O)CCCCCCCN(CCCCCCCOC(=O)C(CCCCCCC)CCCCCCCC)CCCNC(=S)N(C)C] |
| I 328 | 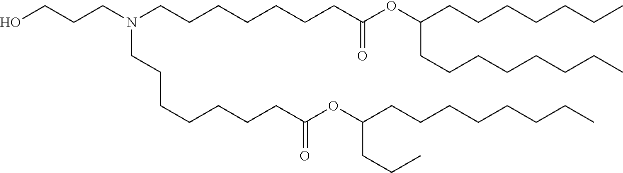 [SMILES: CCCCCCCCC(CCC)OC(=O)CCCCCCCN(CCCO)CCCCCCCC(=O)OC(CCCCCCC)CCCCCCCC] |
| I 329 | 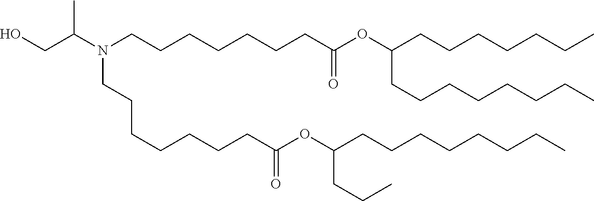 [SMILES: CCCCCCCCC(CCC)OC(=O)CCCCCCCN(CCCCCCCC(=O)OC(CCCCCCC)CCCCCCCC)C(C)CO] |
| I 330 | 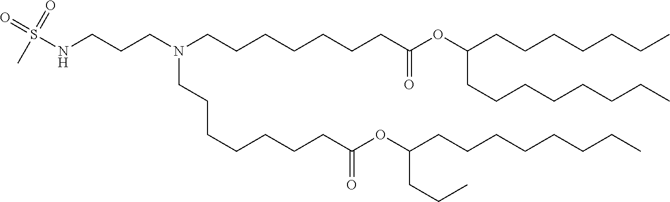 [SMILES: CCCCCCCCC(CCC)OC(=O)CCCCCCCN(CCCCCCCC(=O)OC(CCCCCCC)CCCCCCCC)CCCNS(C)(=O)=O] |
| I 331 | 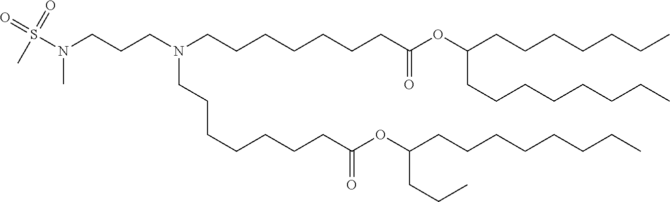 [SMILES: CCCCCCCCC(CCC)OC(=O)CCCCCCCN(CCCCCCCC(=O)OC(CCCCCCC)CCCCCCCC)CCCN(C)S(C)(=O)=O] |
| I 332 | 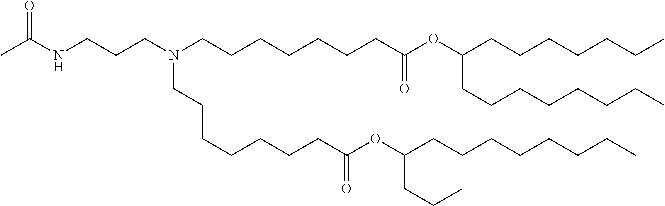 [SMILES: CCCCCCCCC(CCC)OC(=O)CCCCCCCN(CCCCCCCC(=O)OC(CCCCCCC)CCCCCCCC)CCCNC(C)=O] |
| I 333 | 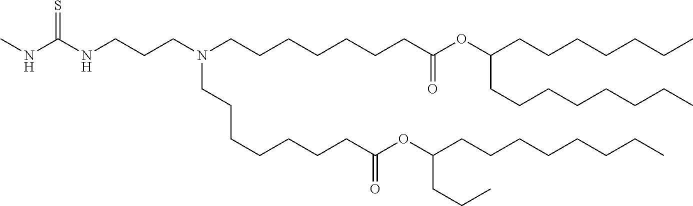 [SMILES: CCCCCCCCC(CCC)OC(=O)CCCCCCCN(CCCCCCCC(=O)OC(CCCCCCC)CCCCCCCC)CCCNC(=S)NC] |
| I 334 | 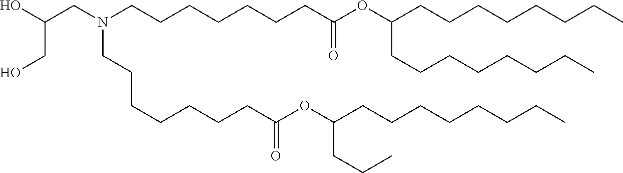 [SMILES: CCCCCCCCC(CCC)OC(=O)CCCCCCCN(CCCCCCCC(=O)OC(CCCCCCCC)CCCCCCCC)CC(O)CO] |
| I 335 | 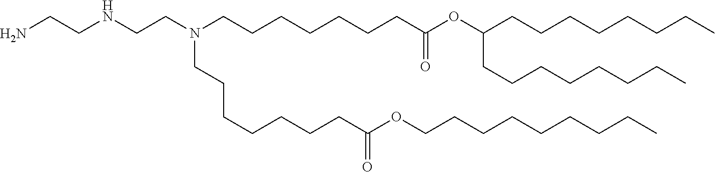 [SMILES: CCCCCCCCCOC(=O)CCCCCCCN(CCCCCCCC(=O)OC(CCCCCCCC)CCCCCCCC)CCNCCN] |
| I 336 | 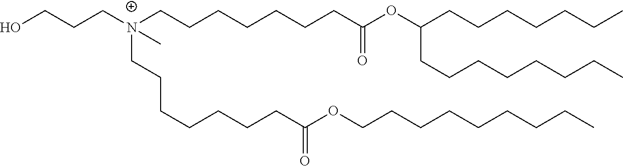 [SMILES: CCCCCCCCCOC(=O)CCCCCCC[N+](C)(CCCO)CCCCCCCC(=O)OC(CCCCCCC)CCCCCCCC] |
| I 337 | 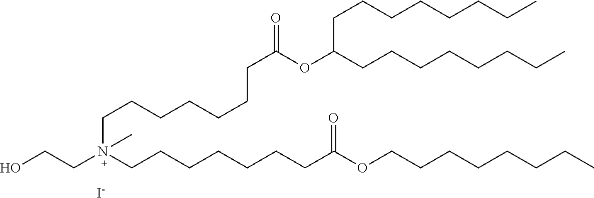 |
| I 338 | 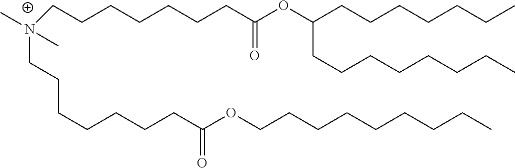 [SMILES: CCCCCCCCCOC(=O)CCCCCCC[N+](C)(C)CCCCCCCC(=O)OC(CCCCCCC)CCCCCCCC] |
| I 339 | 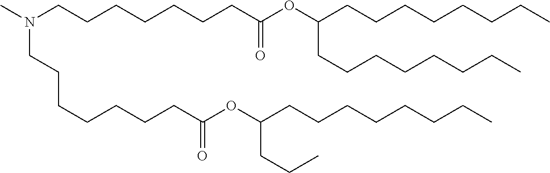 [SMILES: CCCCCCCCC(CCC)OC(=O)CCCCCCCN(C)CCCCCCCC(=O)OC(CCCCCCCC)CCCCCCCC] |
| I 340 | 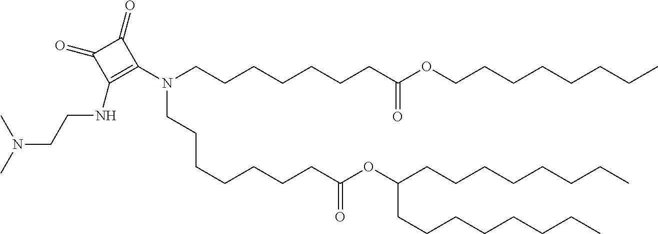 [SMILES: CCCCCCCCOC(=O)CCCCCCCN(CCCCCCCC(=O)OC(CCCCCCCC)CCCCCCCC)c1c(NCCN(C)C)c(=O)c1=O] |
| I 341 | 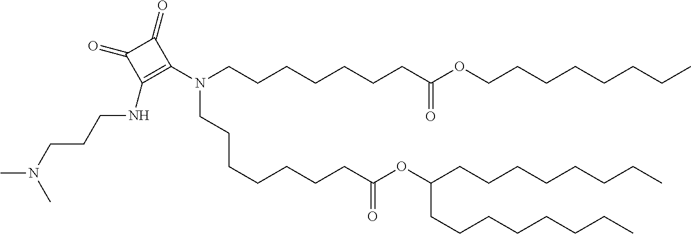 [SMILES: CCCCCCCCOC(=O)CCCCCCCN(CCCCCCCC(=O)OC(CCCCCCCC)CCCCCCCC)c1c(NCCCN(C)C)c(=O)c1=O] |
| I 342 | 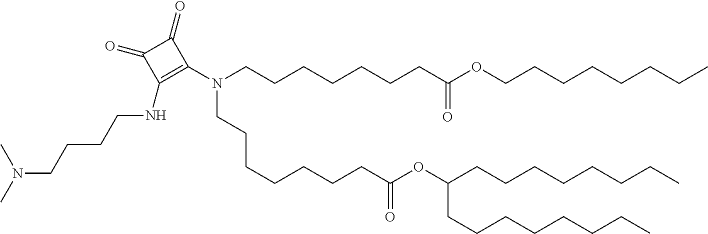 [SMILES: CCCCCCCCOC(=O)CCCCCCCN(CCCCCCCC(=O)OC(CCCCCCCC)CCCCCCCC)c1c(NCCCCN(C)C)c(=O)c1=O] |
| I 343 | 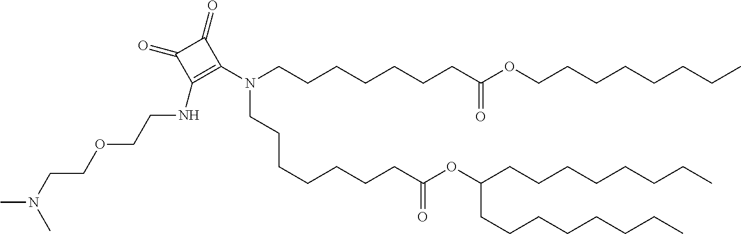 [SMILES: CCCCCCCCOC(=O)CCCCCCCN(CCCCCCCC(=O)OC(CCCCCCCC)CCCCCCCC)c1c(NCCOCCN(C)C)c(=O)c1=O] |
| I 344 | 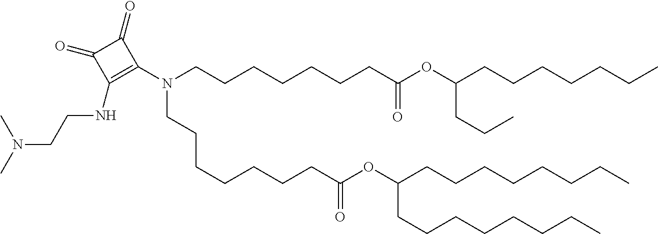 [SMILES: CCCCCCCCC(CCCCCCCC)OC(=O)CCCCCCCN(CCCCCCCC(=O)OC(CCC)CCCCCCC)c1c(NCCN(C)C)c(=O)c1=O] |
| I 345 | 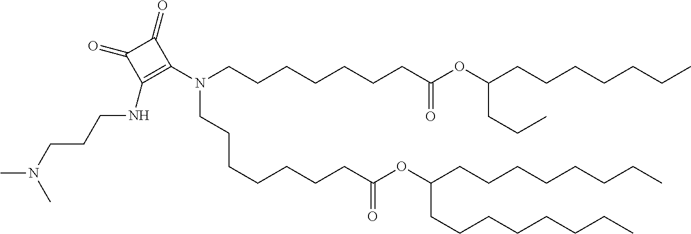 [SMILES: CCCCCCCCC(CCCCCCCC)OC(=O)CCCCCCCN(CCCCCCCC(=O)OC(CCC)CCCCCCC)c1c(NCCCN(C)C)c(=O)c1=O] |
| I 346 | 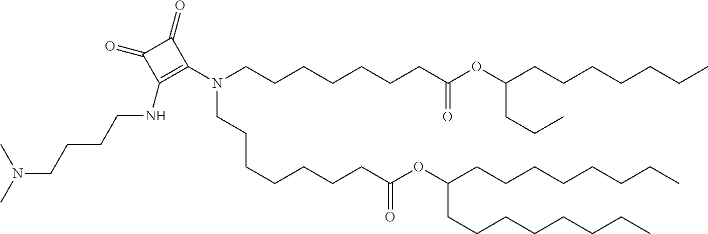 [SMILES: CCCCCCCCC(CCCCCCCC)OC(=O)CCCCCCCN(CCCCCCCC(=O)OC(CCC)CCCCCCC)c1c(NCCCCN(C)C)c(=O)c1=O] |
| I 347 | 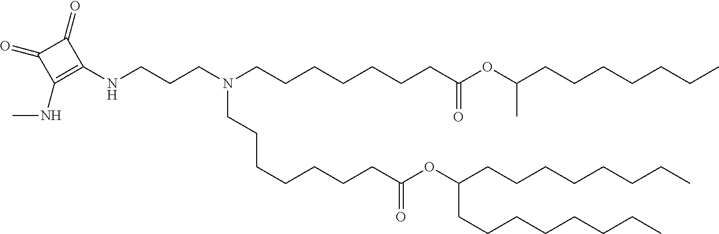 [SMILES: CCCCCCCCC(CCCCCCCC)OC(=O)CCCCCCCN(CCCCCCCC(=O)OC(C)CCCCCCC)CCCNc1c(NC)c(=O)c1=O] |
| I 348 | 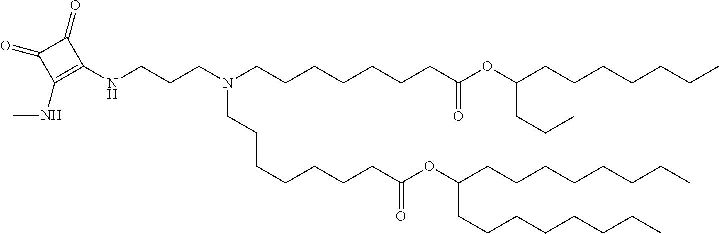 [SMILES: CCCCCCCCC(CCCCCCCC)OC(=O)CCCCCCCN(CCCCCCCC(=O)OC(CCC)CCCCCCC)CCCNc1c(NC)c(=O)c1=O] |
| I 349 | 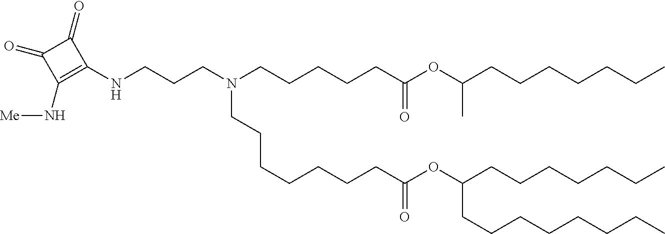 [SMILES: CCCCCCCCC(CCCCCCC)OC(=O)CCCCCCCN(CCCCCC(=O)OC(C)CCCCCCC)CCCNc1c(NC)c(=O)c1=O] |
| I 350 | 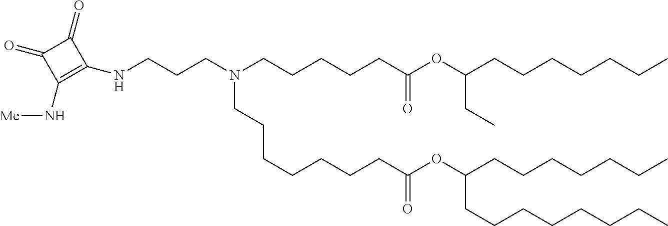 [SMILES: CCCCCCCCC(CCCCCCC)OC(=O)CCCCCCCN(CCCCCC(=O)OC(CC)CCCCCCC)CCCNc1c(NC)c(=O)c1=O] |
| I 351 | 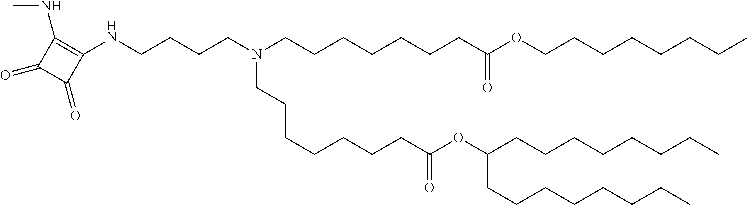 [SMILES: CCCCCCCCOC(=O)CCCCCCCN(CCCCCCCC(=O)OC(CCCCCCCC)CCCCCCCC)CCCCNc1c(NC)c(=O)c1=O] |
| I 352 | 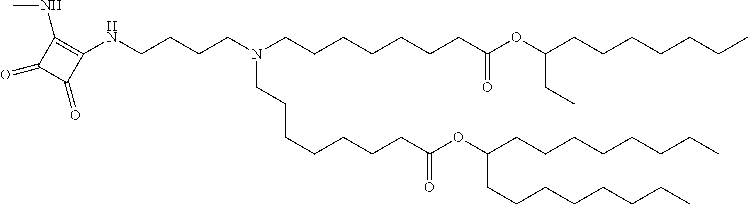 [SMILES: CCCCCCCCC(CCCCCCCC)OC(=O)CCCCCCCN(CCCCCCCC(=O)OC(CC)CCCCCCC)CCCCNc1c(NC)c(=O)c1=O] |
| I 353 | 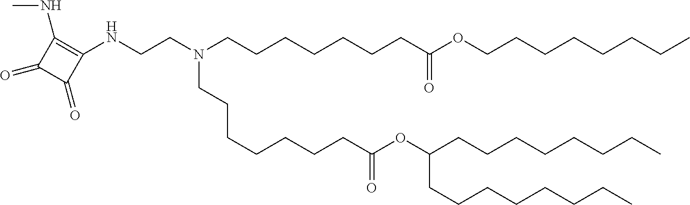 [SMILES: CCCCCCCCOC(=O)CCCCCCCN(CCCCCCCC(=O)OC(CCCCCCCC)CCCCCCCC)CCNc1c(NC)c(=O)c1=O] |
| I 354 | 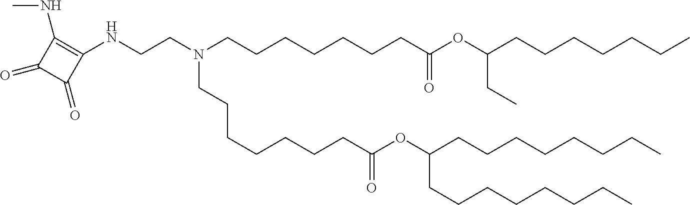 [SMILES: CCCCCCCCC(CCCCCCCC)OC(=O)CCCCCCCN(CCCCCCCC(=O)OC(CC)CCCCCCC)CCNc1c(NC)c(=O)c1=O] |
| I 355 | 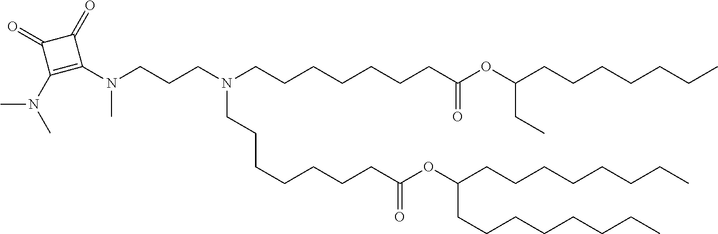 [SMILES: CCCCCCCCC(CCCCCCCC)OC(=O)CCCCCCCN(CCCCCCCC(=O)OC(CC)CCCCCCC)CCCN(C)c1c(N(C)C)c(=O)c1=O] |

In some embodiments, a lipid of the disclosure comprises Compound I-340A:

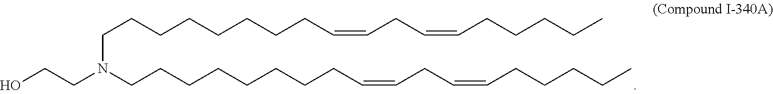 [SMILES: CCCCC/C=C\C/C=C\CCCCCCCCN(CCO)CCCCCCCC/C=C\C/C=C\CCCCC]

The central amine moiety of a lipid according to Formula (I I), (I IA), I (IB), I (II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), or (I VIIId) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some aspects, the ionizable lipids of the present disclosure may be one or more of compounds of formula I (I IX),

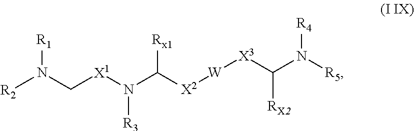

or salts or isomers thereof, wherein

W is

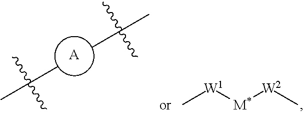

ring A is

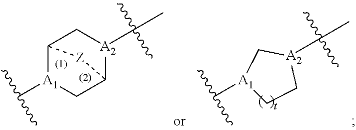

t is 1 or 2;

A₁ and A2 are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)

—CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and -R*MR′; and

n is an integer from 1-6;

wherein when ring A is

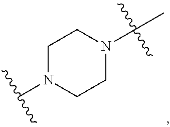 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (I IXa1)-(I IXa8):

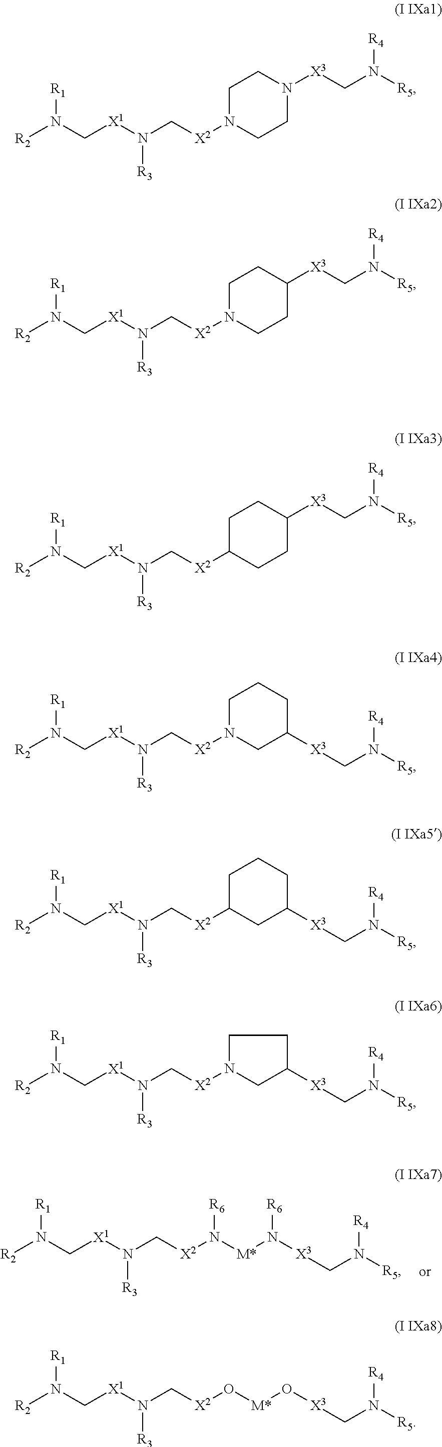

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

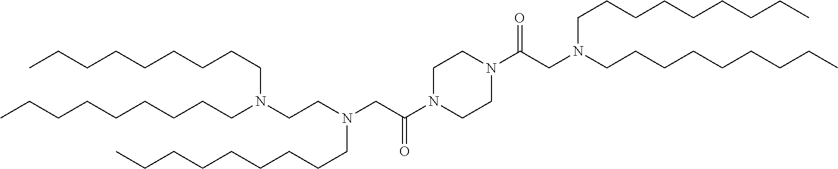 [SMILES: CCCCCCCCCN(CCCCCCCCC)CCN(CCCCCCCCC)CC(=O)N1CCN(C(=O)CN(CCCCCCCCC)CCCCCCCCC)CC1]

(Compound 1-356 (also referred to herein as Compound M), or a salt thereof.
In some embodiments, the ionizable lipid is

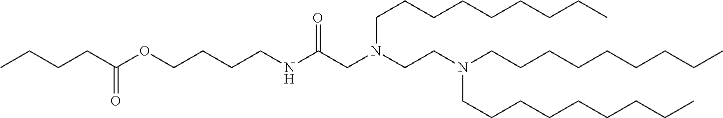 [SMILES: CCCCCCCCCN(CCCCCCCCC)CCN(CCCCCCCCC)CC(=O)NCCCCOC(=O)CCCC]

[Compound I-N], or a salt thereof.

In some embodiments, the ionizable lipid is

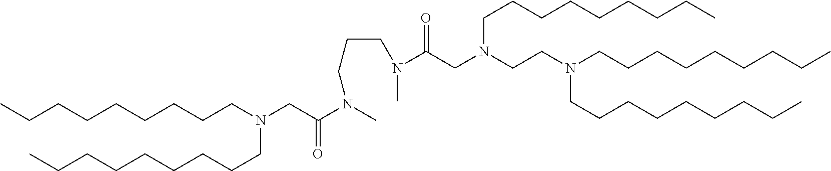 [SMILES: CCCCCCCCCN(CCCCCCCCC)CCN(CCCCCCCCC)CC(=O)N(C)CCCN(C)C(=O)CN(CCCCCCCCC)CCCCCCCCC]

[Compound I-O], or a salt thereof.

In some embodiments, the ionizable lipid is

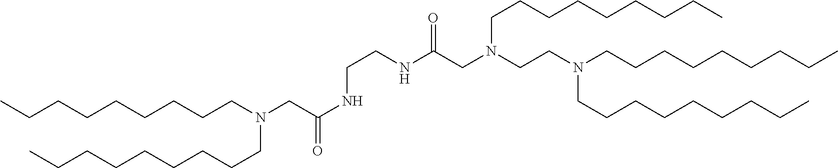 [SMILES: CCCCCCCCCN(CCCCCCCCC)CCN(CCCCCCCCC)CC(=O)NCCNC(=O)CN(CCCCCCCCC)CCCCCCCCC]

[Compound I-P], or a salt thereof.

In some embodiments, the ionizable lipid is

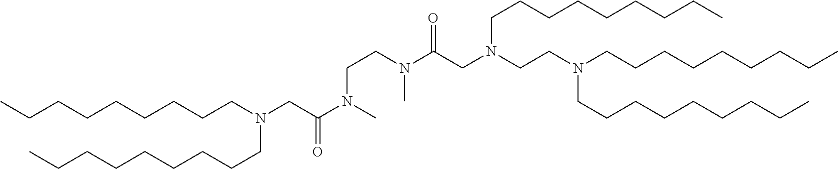 [SMILES: CCCCCCCCCN(CCCCCCCCC)CCN(CCCCCCCCC)CC(=O)N(C)CCN(C)C(=O)CN(CCCCCCCCC)CCCCCCCCC]

[Compound I-Q], or a salt thereof.

The central amine moiety of a lipid according to any of the Formulae herein, e.g. a compound having any of Formula (II), (IIA), (IIB), (II), (IIa), (IIb), (IIc), (IId), (IIe), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some embodiments, the amount the ionizable amino lipid of the invention, e.g. a compound having any of Formula (I), (IA), (IB), (II), (IIa), (IIb), (IIc), (IId), (IIe), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8)) (each of these preceded by the letter I for clarity) ranges from about 1 mol % to 99 mol % in the lipid composition.

In one embodiment, the amount of the ionizable amino lipid of the invention, e.g. a compound having any of Formula (I), (IA), (IB), (II), (IIa), (IIb), (IIc), (IId), (IIe), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the ionizable amino lipid of the invention, e.g. a compound having any of Formula (I), (IA), (IB), (II), (IIa), (IIb), (IIc), (IId), (IIe), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 45 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 40 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 50 mol % in the lipid composition.

In addition to the ionizable amino lipid disclosed herein, e.g. a compound having any of Formula (I), (IA), (IB), (II), (IIa), (IIb), (IIc), (IId), (IIe), MD, (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8), (each of these preceded by the letter I for clarity) the lipid-based composition (e.g., lipid nanoparticle) disclosed herein can comprise additional components such as cholesterol and/or cholesterol analogs, non-cationic helper lipids, structural lipids, PEG-lipids, and any combination thereof.

Additional ionizable lipids of the invention can be selected from the non-limiting group consisting of 3-(didodecylamino)-N1,N1,4-tridodecyl-1-piperazineethanamine (KL¹⁰),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
heptatriaconta-6,9,28,31-tetraen-19-yl 4-(dimethylamino)butanoate (DLin-MC3-DMA),
2,2-dilinoleyl-4-(2-dimethylaminoethyl)-[1,3]-dioxolane (DLin-KC2-DMA),
1,2-dioleyloxy-N,N-dimethylaminopropane (DODMA), (13Z,165Z)—N,N-dimethyl-3-nonydocosa-13-16-dien-1-amine (L608),
2-({8-[3β)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and
(2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids of the invention can also be the compounds disclosed in International Publication No. WO 2017/075531 A1, hereby incorporated by reference in its entirety. For example, the ionizable amino lipids include, but not limited to:

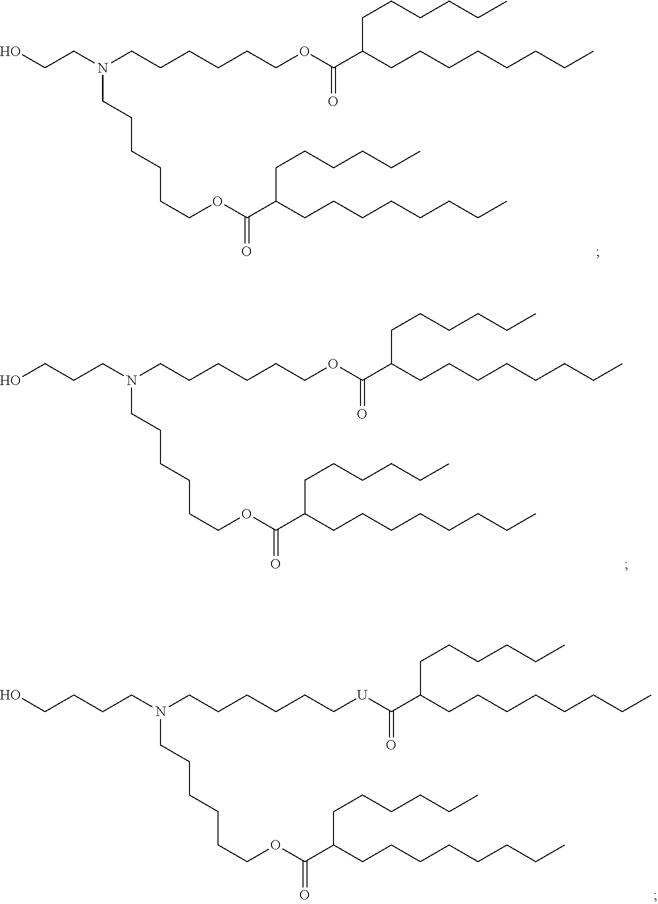

and any combination thereof.

Ionizable lipids of the invention can also be the compounds disclosed in International Publication No. WO 2015/199952 A1, hereby incorporated by reference in its entirety. For example, the ionizable amino lipids include, but not limited to:

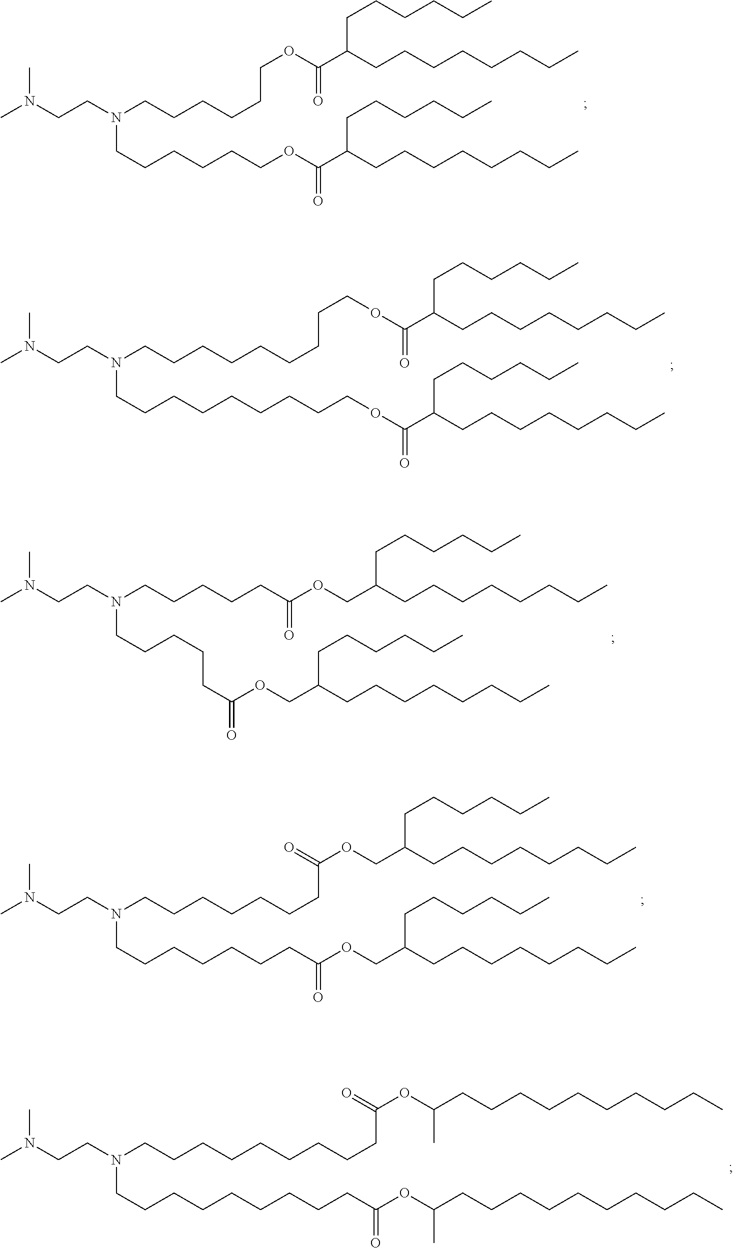

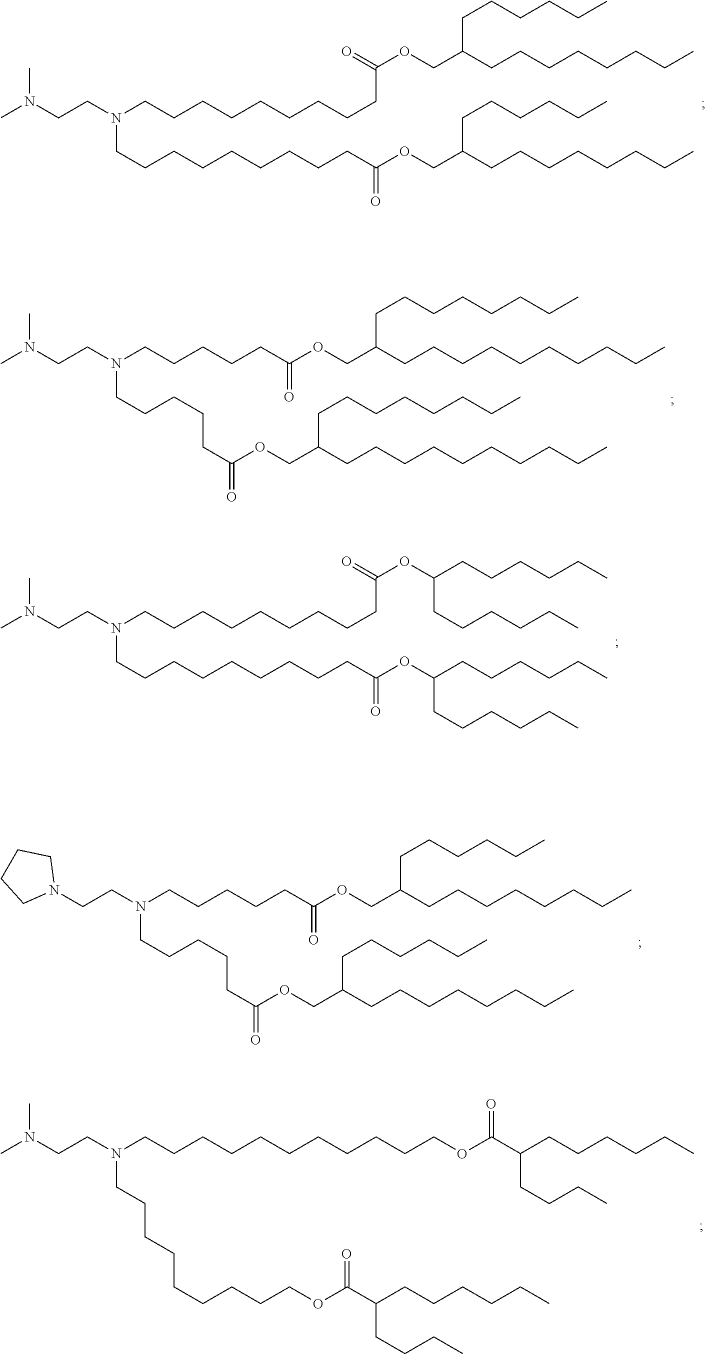

and any combination thereof.

In any of the foregoing or related aspects, the ionizable lipid of the LNP of the disclosure comprises a compound included in any e.g. a compound having any of Formula (I), (IA), (IB), (II), (IIa), (IIb), (IIc), (IId), (IIe), (IIf), (IIg), (III), (VI), (VI-a), (VII), (VIII), (Vila), (Villa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity).

In any of the foregoing or related aspects, the ionizable lipid of the LNP of the disclosure comprises a compound comprising any of Compound Nos. I 1-356.

In any of the foregoing or related aspects, the ionizable lipid of the LNP of the disclosure comprises at least one compound selected from the group consisting of: Compound Nos. I 18 (also referred to as Compound X or Compound II), I 25 (also referred to as Compound Y), I 48, I 50, I 109, I 111, I 113, I 181, I 182, I 244, I 292, I 301, I 321, I 322, I 326, I 328, I 330, I 331, and I 332. In another embodiment, the ionizable lipid of the LNP of the disclosure comprises a compound selected from the group consisting of: Compound Nos. I 18 (also referred to as Compound X or Compound II), I 25 (also referred to as Compound Y), I 48, I 50, I 109, I 111, I 181, I 182, I 292, I 301, I 321, I 326, I 328, and I 330. In another embodiment, the ionizable lipid of the LNP of the disclosure comprises a compound selected from the group consisting of: Compound Nos. I 182, I 301, I 321, and I 326.

In any of the foregoing or related aspects, the synthesis of compounds of the invention, e.g. compounds comprising any of Compound Nos. 1-356, follows the synthetic descriptions in U.S. Provisional Patent Application No. 62/733,315, filed Sep. 19, 2018.

Representative Synthetic Routes:

### Compound 1-182: Heptadecan-9-yl 8((3((2-(methylamino)-3,4-dioxocyclobut-1-en-1-yl)amino)propyl)(8-(nonyloxy)-8-oxooctyl)amino)octanoate 3-Methoxy-4-(methylamino)cyclobut-3-ene-1,2-dione

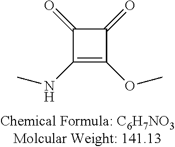

To a solution of 3,4-dimethoxy-3-cyclobutene-1,2-dione (1 g, 7 mmol) in 100 mL diethyl ether was added a 2M methylamine solution in THF (3.8 mL, 7.6 mmol) and a ppt. formed almost immediately. The mixture was stirred at rt for 24 hours, then filtered, the filter solids washed with diethyl ether and air-dried. The filter solids were dissolved in hot EtOAc, filtered, the filtrate allowed to cool to room temp., then cooled to 0° C. to give a ppt. This was isolated via filtration, washed with cold EtOAc, air-dried, then dried under vacuum to give 3-methoxy-4-(methylamino)cyclobut-3-ene-1,2-dione (0.70 g, 5 mmol, 73%) as a white solid. ¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### Heptadecan-9-yl 8-((3-((2-(methylamino)-3,4-dioxocyclobut-1-en-1-yl)amino)propyl)(8-(nonyloxy)-8-oxooctyl)amino)octanoate

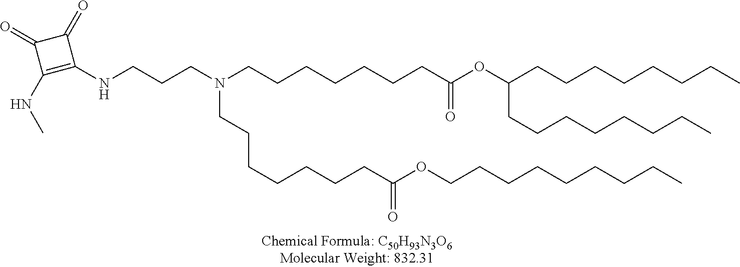 [SMILES: CCCCCCCCCOC(=O)CCCCCCCN(CCCCCCCC(=O)OC(CCCCCCCC)CCCCCCCC)CCCNc1c(NC)c(=O)c1=O]

To a solution of heptadecan-9-yl 8-((3-aminopropyl)(8-(nonyloxy)-8-oxooctyl)amino)octanoate (200 mg, 0.28 mmol) in 10 mL ethanol was added 3-methoxy-4-(methylamino)cyclobut-3-ene-1,2-dione (39 mg, 0.28 mmol) and the resulting colorless solution stirred at rt for 20 hours after which no starting amine remained by LC/MS. The solution was concentrated in vacuo and the residue purified by silica gel chromatography (0-100% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-(nonyloxy)-8-oxooctyl)amino)octanoate (138 mg, 0.17 mmol, 60%) as a gummy white solid. UPLC/ELSD: RT=3. min MS (ES): m/z (MH⁺) 833.4 for C₅₁H₉₅N₃O₆. ¹H NMR (300 MHz, CDCl₃) δ: ppm 7.86 (br. s., 1H); 4.86 (quint., 1H, J=6 Hz); 4.05 (t, 2H, J=6 Hz); 3.92 (d, 2H, J=3 Hz); 3.20 (s, 6H); 2.63 (br. s, 2H); 2.42 (br. s, 3H); 2.28 (m, 4H); 1.74 (br. s, 2H); 1.61 (m, 8H); 1.50 (m, 5H); 1.41 (m, 3H); 1.25 (br. m, 47H); 0.88 (t, 9H, J=7.5 Hz).

### Compound I-301: Heptadecan-9-yl 8-((3-((2-(methylamino)-3,4-dioxocyclobut-1-en-1-yl)amino)propyl)(8-oxo-8-(undecan-3-yloxy)octyl)amino)octanoate

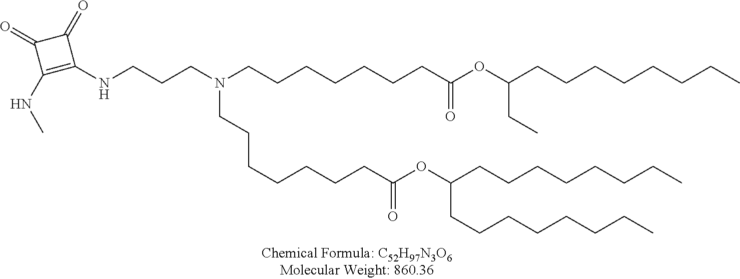 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

Compound I-301 was prepared analogously to compound 182 except that heptadecan-9-yl 8-((3-aminopropyl)(8-oxo-8-(undecan-3-yloxy)octyl)amino)octanoate (500 mg, 0.66 mmol) was used instead of heptadecan-9-yl 8-((3-aminopropyl)(8-(nonyloxy)-8-oxooctyl)amino)octanoate. Following an aqueous workup the residue was purified by silica gel chromatography (0-50% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-oxo-8-(undecan-3-yloxy)octyl)amino)octanoate (180 mg, 32%) as a white waxy solid. HPLC/UV (254 nm): RT=6.77 min MS (CI): m/z (MH⁺) 860.7 for C₅₂H₉₇N₃O₆. ¹H NMR (300 MHz, CDCl₃): δ ppm 4.86-4.79 (m, 2H); 3.66 (bs, 2H); 3.25 (d, 3H, J=4.9 Hz); 2.56-2.52 (m, 2H); 2.42-2.37 (m, 4H); 2.28 (dd, 4H, J=2.7 Hz, 7.4 Hz); 1.78-1.68 (m, 3H); 1.64-1.50 (m, 16H); 1.48-1.38 (m, 6H); 1.32-1.18 (m, 43H); 0.88-0.84 (m, 12H).

(ii) Cholesterol/Structural Lipids

In some embodiments, the LNPs described herein comprise one or more structural lipids. As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

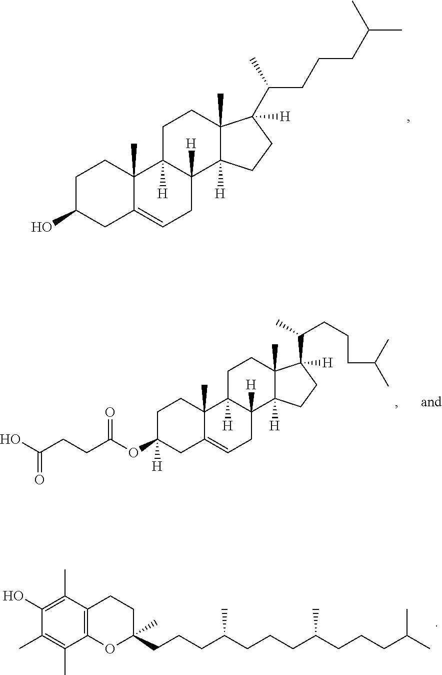

The LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. Structural lipids can include, but are not limited to, sterols (e.g., phytosterols or zoosterols).

In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds S1-148 in Tables 1-16 herein.

In certain embodiments, the structural lipid is cholesterol. In certain embodiments, the structural lipid is an analog of cholesterol.

In certain embodiments, the structural lipid is alpha-tocopherol.

In an aspect, the structural lipid of the invention features a compound having the structure of Formula SI:

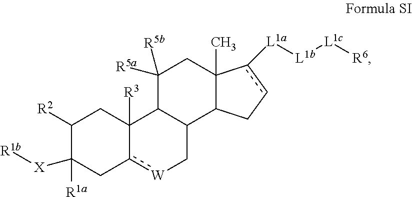

where

R^{1a} is H, optionally substituted C₁-C₆ alkyl, optionally substituted C₂-C₆ alkenyl, or optionally substituted C₂-C₆ alkynyl;

X is O or S;

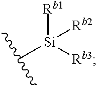

R^{1b} is H, optionally substituted C₁-C₆ alkyl, or

each of R^{b1}, R^{b2}, and R^{b3} is, independently, optionally substituted C₁-C₆ alkyl or optionally substituted C₆-C₁₀ aryl;

R² is H or OR^{A}, where R^{A} is H or optionally substituted C₁-C₆ alkyl;

R³ is H or

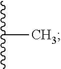

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

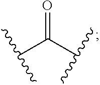 [SMILES: *C(*)=O]

L^{1a} is absent,

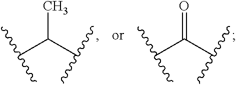

L^{1b} is absent,

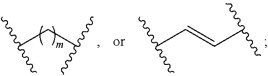

m is 1, 2, or 3;

L^{1c} is absent,

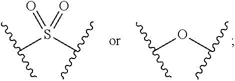

and

R⁶ is optionally substituted C₃-C₁₀ cycloalkyl, optionally substituted C₃-C₁₀ cycloalkenyl, optionally substituted C₆-C₁₀ aryl, optionally substituted C₂-C₉ heterocyclyl, or optionally substituted C₂-C₉ heteroaryl,
or a pharmaceutically acceptable salt thereof.

In some embodiments, the compound has the structure of Formula SIa:

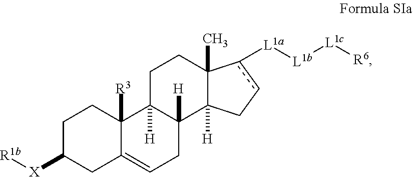

or a pharmaceutically acceptable salt thereof.

In some embodiments, the compound has the structure of Formula SIb:

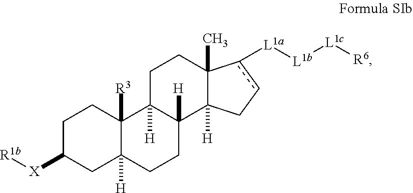

or a pharmaceutically acceptable salt thereof.

In some embodiments, the compound has the structure of Formula SIc:

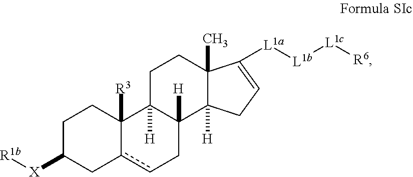

or a pharmaceutically acceptable salt thereof.

In some embodiments, the compound has the structure of Formula SId:

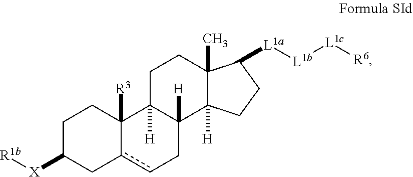

or a pharmaceutically acceptable salt thereof.

In some embodiments, L^{1a} is absent. In some embodiments, L^{1a} is

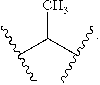 [SMILES: *C(*)C]

In some embodiments, L^{1a} is

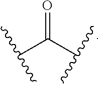 [SMILES: *C(*)=O]

In some embodiments, L^{1b} is absent. In some embodiments, L^{1b} is

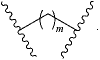

In some embodiments, L^{1b} is

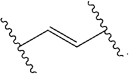 [SMILES: */C=C/*]

In some embodiments, m is 1 or 2. In some embodiments, m is 1. In some embodiments, m is 2.

In some embodiments, L^{1c} is absent. In some embodiments, L^{1c} is

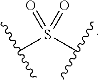 [SMILES: *S(*)(=O)=O]

In some embodiments, L^{1c} is

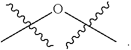 [SMILES: *O*]

In some embodiments, R⁶ is optionally substituted C₆-C₁₀ aryl.

In some embodiments, R⁶ is

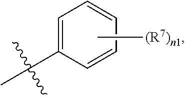

where

n1 is 0, 1, 2, 3, 4, or 5; and

each R⁷ is, independently, halo or optionally substituted C₁-C₆ alkyl.

In some embodiments, each R⁷ is, independently,

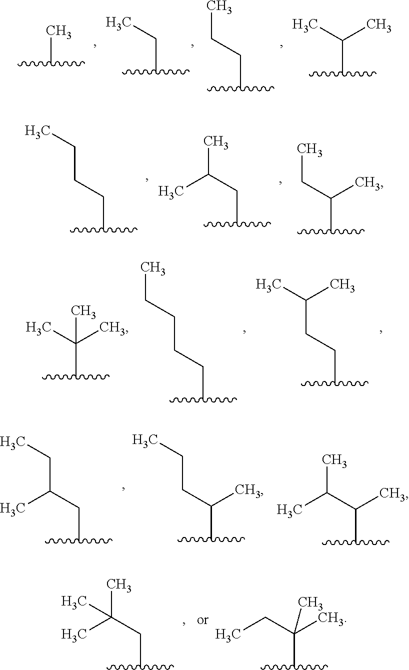

In some embodiments, n1 is 0, 1, or 2. In some embodiments, n is 0. In some embodiments, n1 is 1. In some embodiments, n1 is 2.

In some embodiments, R⁶ is optionally substituted C₃-C₁₀ cycloalkyl.

In some embodiments, R⁶ is optionally substituted C₃-C₁₀ monocycloalkyl.

In some embodiments, R⁶ is

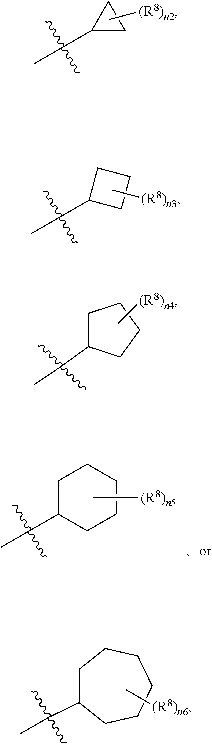

where

n2 is 0, 1, 2, 3, 4, or 5;

n3 is 0, 1, 2, 3, 4, 5, 6, or 7;

n4 is 0, 1, 2, 3, 4, 5, 6, 7, 8, or 9;

n5 is 0, 1, 2, 3, 4, 5, 6, 7, 8, 9, 10, or 11;

n6 is 0, 1, 2, 3, 4, 5, 6, 7, 8, 9, 10, 11, 12, or 13; and

each R⁸ is, independently, halo or optionally substituted C₁-C₆ alkyl.

In some embodiments, each R⁸ is, independently,

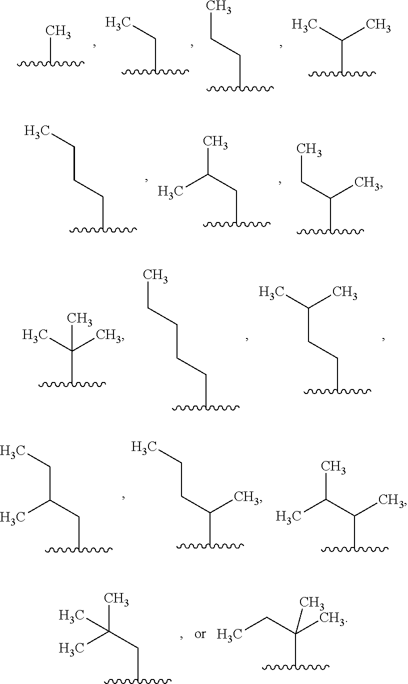

In some embodiments, R⁶ is optionally substituted C₃-C₁₀ polycycloalkyl.

In some embodiments, R⁶ is

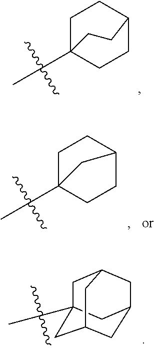 [SMILES: *C12CCC(CC1)CC2 ; *C12CCC(CC1)C2 ; *C12CC3CC(CC(C3)C1)C2]

In some embodiments, R⁶ is optionally substituted C₃-C₁₀ cycloalkenyl.

In some embodiments, R⁶ is

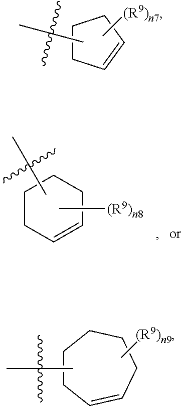

where

n7 is 0, 1, 2, 3, 4, 5, 6, or 7;

n8 is 0, 1, 2, 3, 4, 5, 6, 7, 8, or 9;

n9 is 0, 1, 2, 3, 4, 5, 6, 7, 8, 9, 10, or 11; and

each R⁹ is, independently, halo or optionally substituted C₁-C₆ alkyl.

In some embodiments, R⁶ is

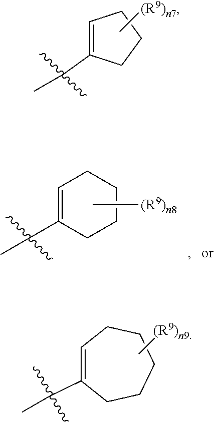

In some embodiments, each R⁹ is, independently,

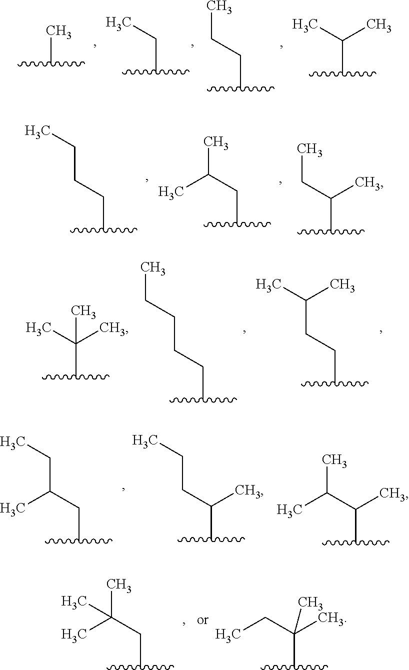

In some embodiments, R⁶ is optionally substituted C₂-C₉ heterocyclyl.

In some embodiments, R⁶ is

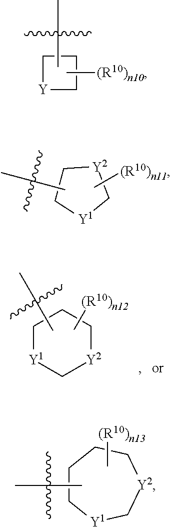

where

n10 is 0, 1, 2, 3, 4, or 5;

n11 is 0, 1, 2, 3, 4, or 5;

n12 is 0, 1, 2, 3, 4, 5, 6, or 7;

n13 is 0, 1, 2, 3, 4, 5, 6, 7, 8, or 9;

each R¹⁰ is, independently, halo or optionally substituted C₁-C₆ alkyl; and

each of Y¹ and Y² is, independently, O, S, NR^{B}, or CR^{11a}R^{11b},

where R^{B} is H or optionally substituted C₁-C₆ alkyl;

each of R^{11a} and R^{11b} is, independently, H, halo, or optionally substituted C₁-C₆ alkyl; and

if Y² is CR^{11a}R^{11b}, then Y1 is O, S, or NR^{B}.

In some embodiments, Y¹ is O.

In some embodiments, Y² is O. In some embodiments, Y² is CR^{11a}R^{11b}.

In some embodiments, each R¹⁰ is, independently,

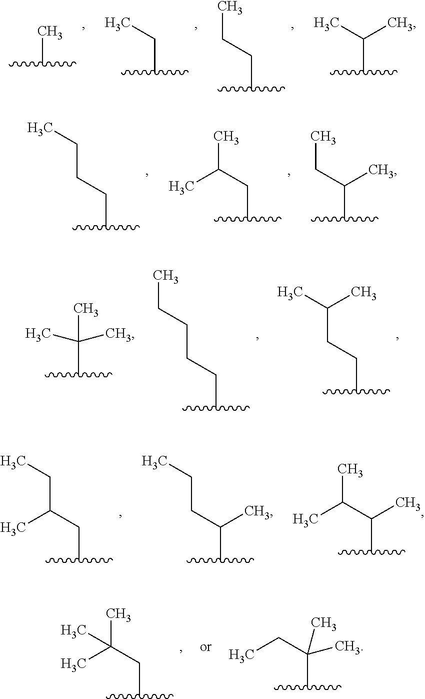

In some embodiments, R⁶ is optionally substituted C₂-C₉ heteroaryl.

In some embodiments, R⁶ is

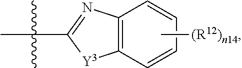

where

Y³ is NR^{c}, O, or S

n14 is 0, 1, 2, 3, or 4;

R^{c} is H or optionally substituted C₁-C₆ alkyl; and

each R¹² is, independently, halo or optionally substituted C₁-C₆ alkyl.

In some embodiments, R⁶ is

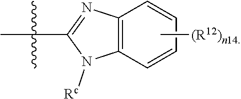

In some embodiments, R⁶ is

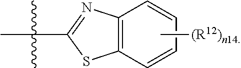

In an aspect, the structural lipid of the invention features a compound having the structure of Formula SII:

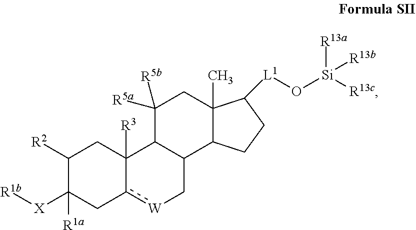

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

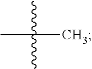

[symbol]

represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the

atom to which each is attached, combine to form

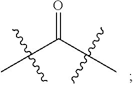 [SMILES: *C(*)=O]

L¹ is optionally substituted C₁-C₆ alkylene; and

each of R^{13a}, R^{13b}, and R^{13c} is, independently, optionally substituted C₁-C₆ alkyl or optionally substituted C₆-C₁₀ aryl, or a pharmaceutically acceptable salt thereof.

In some embodiments, the compound has the structure of Formula SIIa:

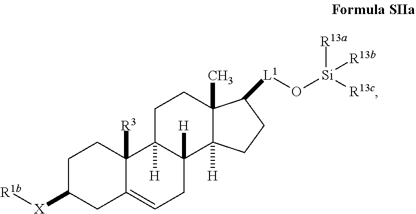

or a pharmaceutically acceptable salt thereof.

In some embodiments, the compound has the structure of Formula SIIb:

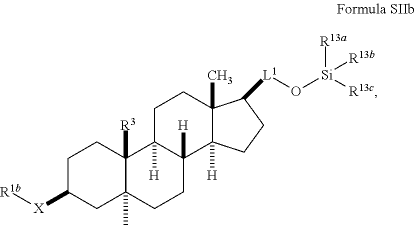

or a pharmaceutically acceptable salt thereof.

In some embodiments, L¹ is

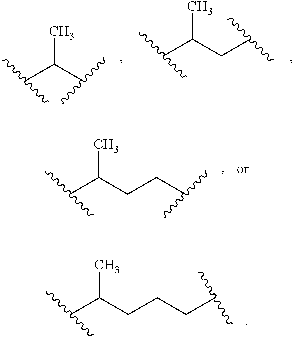

In some embodiments, each of R^{13a}, R^{13b}, and R^{13c} is, independently,

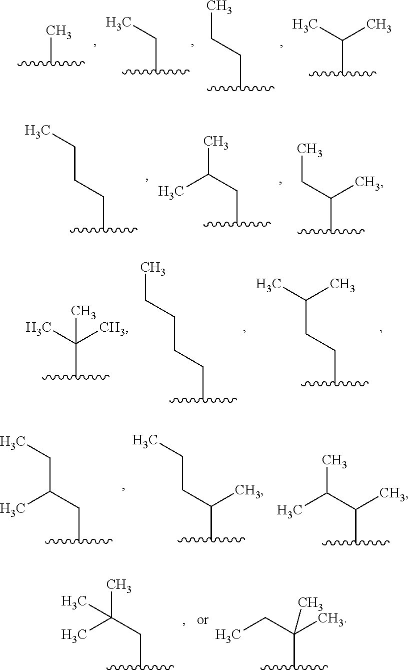

In an aspect, the structural lipid of the invention features a compound having the structure of Formula SIII:

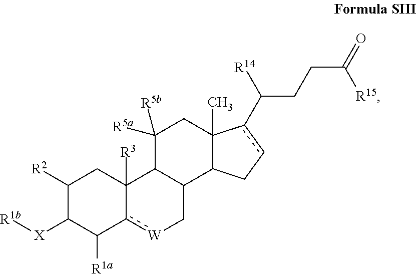

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

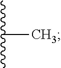

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, hydroxyl, optionally substituted C₁-C₆ alkyl, —OS(O)₂R^{4c}, where R^{4c} is optionally substituted C₁-C₆ alkyl or optionally substituted C₆-C₁₀ aryl;

each of R^{5a} and R^{5b} is, independently, H or OR^{A}, or R^{5a} and R^{5b}, together with the atom to which each is attached, combine to form

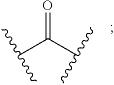 [SMILES: *C(*)=O]

R¹⁴ is H or C₁-C₆ alkyl; and

R¹⁵ is where

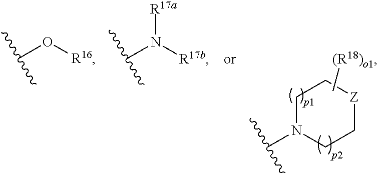

R¹⁶ is H or optionally substituted C₁-C₆ alkyl;
R^{17b} is H, OR^{17c}, optionally substituted C₆-C₁₀ aryl, or optionally substituted C₁-C₆ alkyl;

R^{17c} is H or optionally substituted C₁-C₆ alkyl;

o1 is 0, 1, 2, 3, 4, 5, 6, 7, or 8;

p1 is 0, 1, or 2;

p2 is 0, 1, or 2;

Z is CH₂O, S, or NR^{D}, where R^{D} is H or optionally substituted C₁-C₆ alkyl; and

each R¹⁸ is, independently, halo or optionally substituted C₁-C₆ alkyl, or a pharmaceutically acceptable salt thereof.

In some embodiments, the compound has the structure of Formula SIIIa:

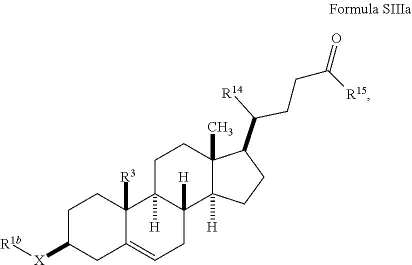

or a pharmaceutically acceptable salt thereof.

In some embodiments, the compound has the structure of Formula SIIIb:

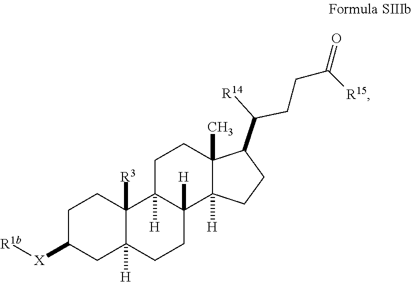

or a pharmaceutically acceptable salt thereof.

In some embodiments, R¹⁴ is H,

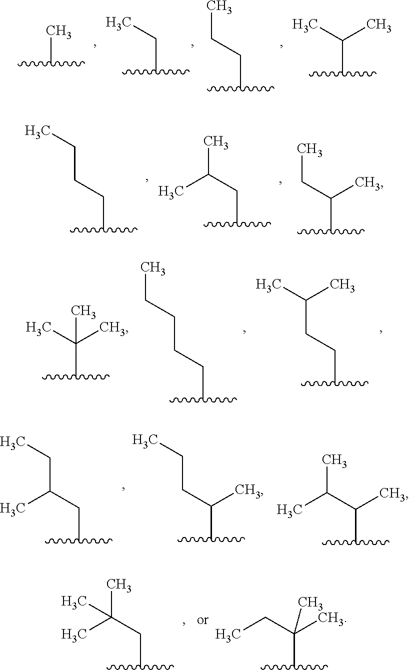

In some embodiments, R¹⁴ is

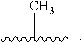

In some embodiments, R¹⁵ is

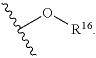

In some embodiments, R¹⁵ is

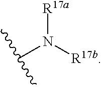

In some embodiments, R¹⁶ is H. In some embodiments, R¹⁶ is

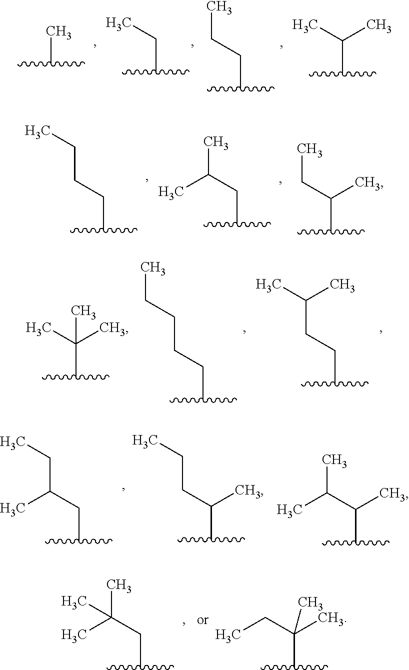

In some embodiments, R^{17a} is H. In some embodiments, R^{17a} is optionally substituted C₁-C₆ alkyl.

In some embodiments, R^{17b} is H. In some embodiments, R^{17b} optionally substituted C₁-C₆ alkyl. In some embodiments, R^{17b} is OR^{17c}.

In some embodiments, R^{17c} is H,

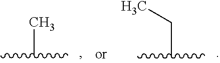

In some embodiments, R^{17c} is H.

In some embodiments, R^{17e} is

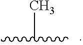

In some embodiments, R¹⁵ is

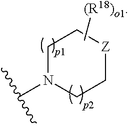

In some embodiments, each R¹⁸ is, independently,

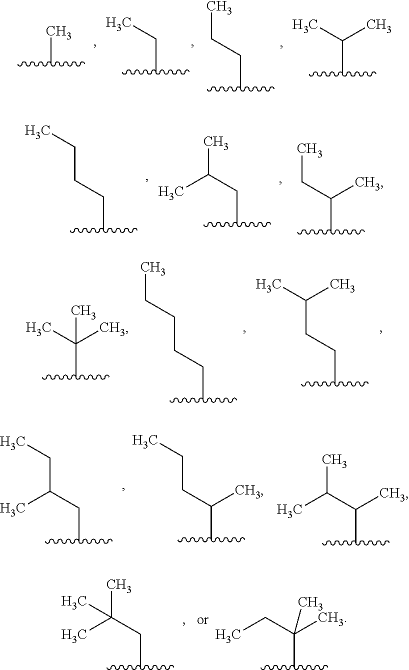

In some embodiments, Z is CH₂. In some embodiments, Z is O. In some embodiments, Z is NR^{D}.

In some embodiments, o1 is 0, 1, 2, 3, 4, 5, or 6.

In some embodiments, o1 is 0. In some embodiments, o1 is 1. In some embodiments, o1 is 2. In some embodiments, o1 is 3. In some embodiments, o1 is 4. In some embodiments, o1 is 5. In some embodiments, o1 is 6.

In some embodiments, p1 is 0 or 1. In some embodiments, p1 is 0. In some embodiments, p1 is 1.

In some embodiments, p2 is 0 or 1. In some embodiments, p2 is 0. In some embodiments, p2 is 1.

In an aspect, the structural lipid of the invention features a compound having the structure of Formula SIV:

where

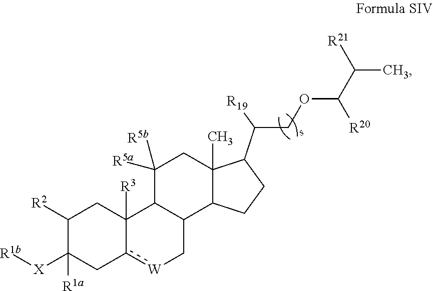

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

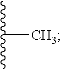

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

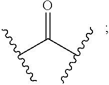 [SMILES: *C(*)=O]

s is 0 or 1;

R¹⁹ is H or C₁-C₆ alkyl;

R²⁰ is C₁-C₆ alkyl;

R²¹ is H or C₁-C₆ alkyl,

or a pharmaceutically acceptable salt thereof.

In some embodiments, the compound has the structure of Formula SIVa:

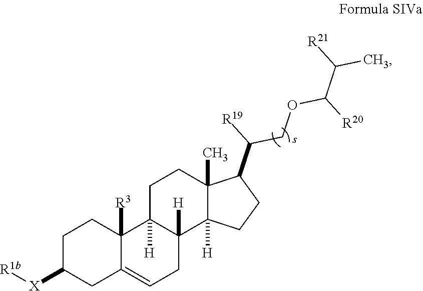

or a pharmaceutically acceptable salt thereof.

In some embodiments, the compound has the structure of Formula SIVb:

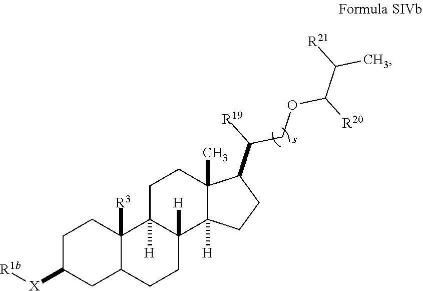

or a pharmaceutically acceptable salt thereof.

In some embodiments, R¹⁹ is H

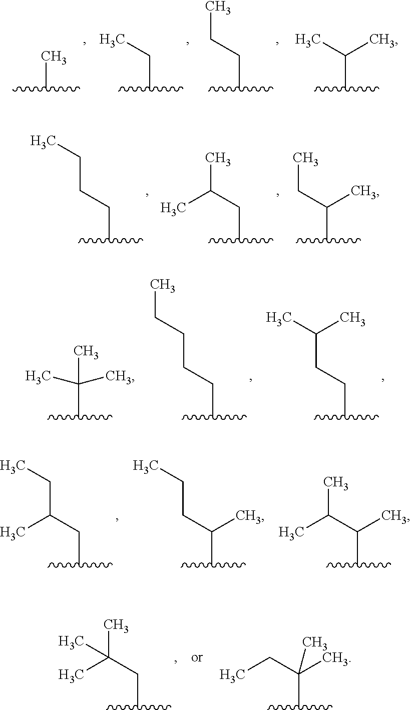

In some embodiments, R¹⁹ is

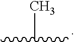

In some embodiments, R²⁰ is,

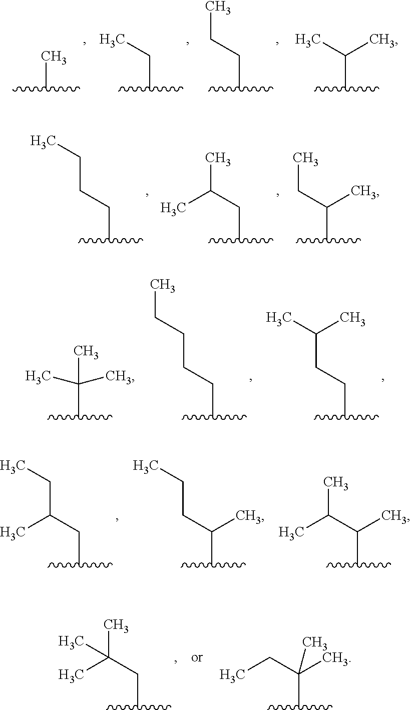

In some embodiments, R²¹ is H,

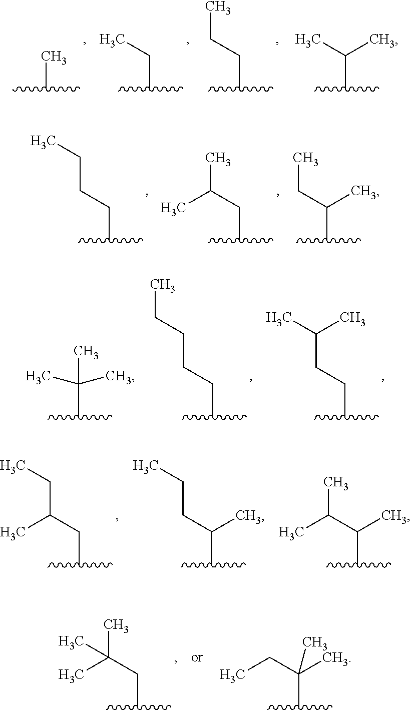

In an aspect, the structural lipid of the invention features, a compound having the structure of Formula SV:

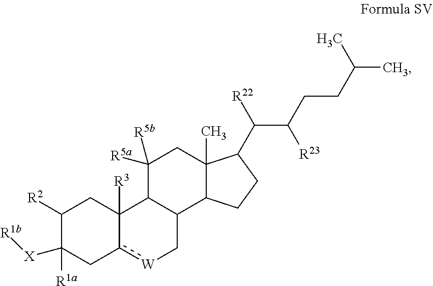

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

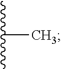

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

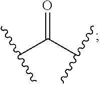 [SMILES: *C(*)=O]

R²² is H or C₁-C₆ alkyl; and

R²³ is halo, hydroxyl, optionally substituted C₁-C₆ alkyl, or optionally substituted C₁-C₆ heteroalkyl,

or a pharmaceutically acceptable salt thereof.

In some embodiments, the compound has the structure of Formula SVa:

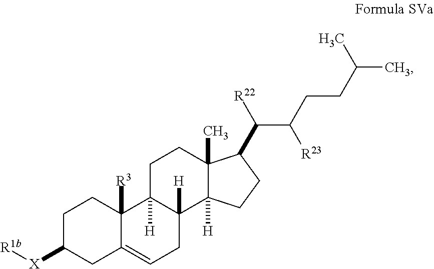

or a pharmaceutically acceptable salt thereof.

In some embodiments, the compound has the structure of Formula SVb:

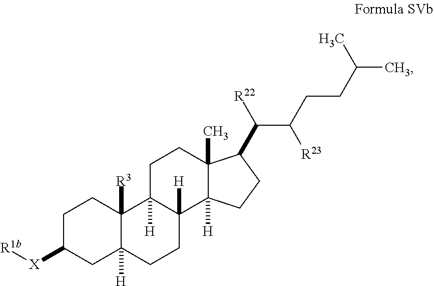

or a pharmaceutically acceptable salt thereof.

In some embodiments, R²² is H,

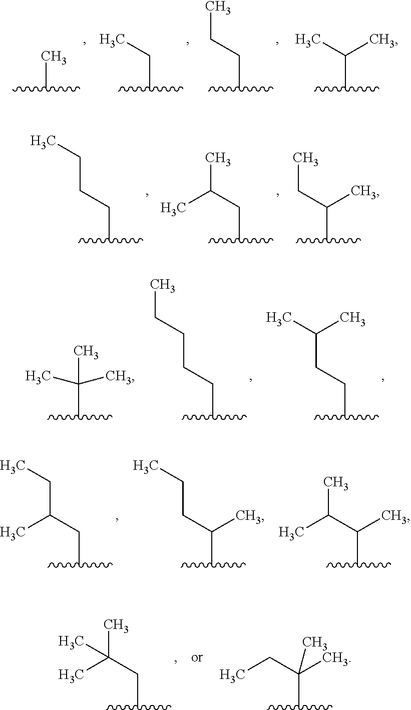

In some embodiments, R²² is

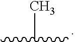

In some embodiments, R²³ is

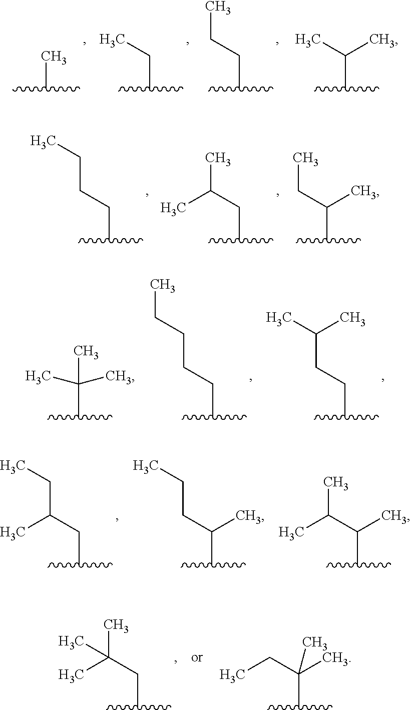

In an aspect, the structural lipid of the invention features a compound having the structure of Formula SVI:

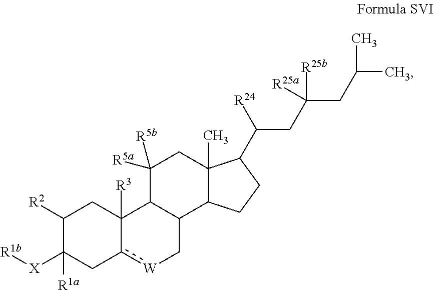

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

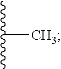

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

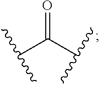 [SMILES: *C(*)=O]

R²⁴ is H or C₁-C₆ alkyl; and

each of R^{25a} and R^{25b} is C₁-C₆ alkyl,

or a pharmaceutically acceptable salt thereof.

In some embodiments, the compound has the structure of Formula SVIa:

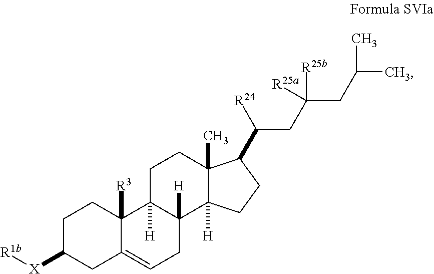

or a pharmaceutically acceptable salt thereof.

In some embodiments, the compound has the structure of Formula SVIb:

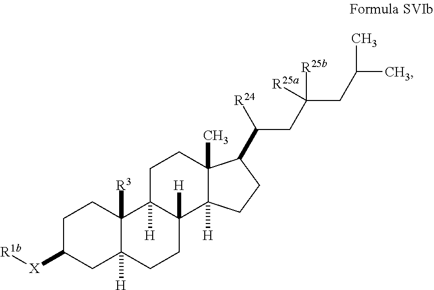

or a pharmaceutically acceptable salt thereof.

In some embodiments, R²⁴ is H,

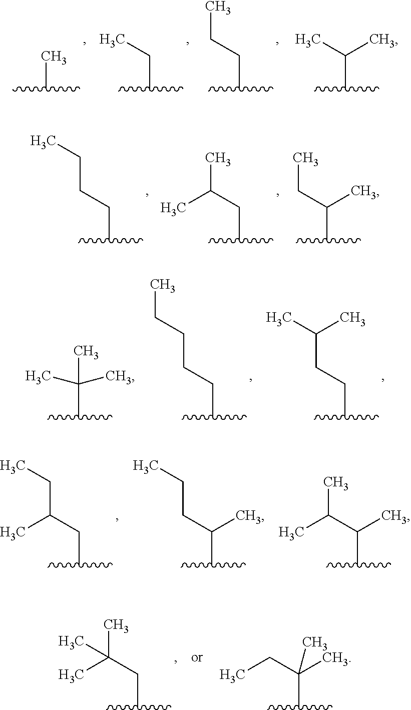

In some embodiments, R²⁴ is

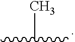

In some embodiments, each of R^{25a} and R^{25b} is, independently,

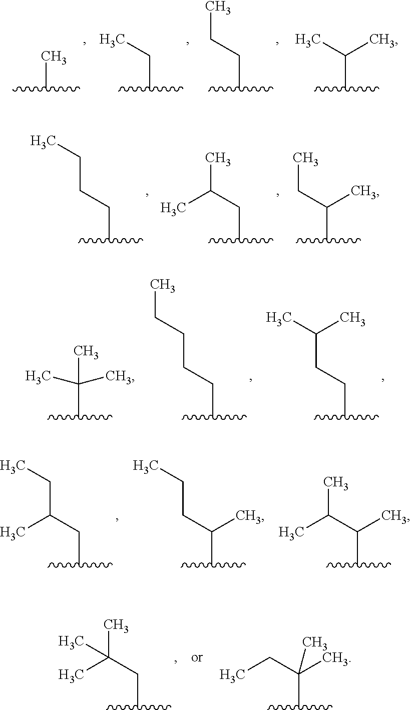

In an aspect, the structural lipid of the invention features a compound having the structure of Formula SVII:

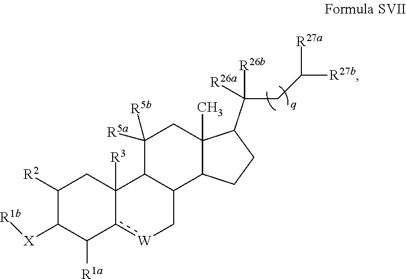

where

R^{1a} is H, optionally substituted C₁-C₆ alkyl, optionally substituted C₂-C₆ alkenyl, optionally substituted C₂-C₆ alkynyl, or

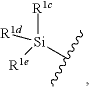

where each of R^{1c}, R^{1d}, and R^{1e} is, independently, optionally substituted C₁-C₆ alkyl or optionally substituted C₆-C₁₀ aryl;

X is O or S;

R^{1b} is H or optionally substituted C₁-C₆ alkyl;

R² is H or OR^{A}, where R^{A} is H or optionally substituted C₁-C₆ alkyl;

R³ is H or

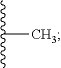

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

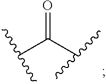 [SMILES: *C(*)=O]

q is 0 or 1;

each of R^{26a} and R^{26b} is, independently, H or optionally substituted C₁-C₆ alkyl, or R^{26a} and R^{26b}, together with the atom to which each is attached, combine to form

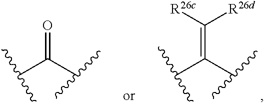

where each of R^{26c} and R²⁶ is, independently, H or optionally substituted C₁-C₆ alkyl; and

each of R^{27a} and R^{27b} is H, hydroxyl, or optionally substituted C₁-C₆ alkyl,

or a pharmaceutically acceptable salt thereof.

In some embodiments, the compound has the structure of Formula SVIIa:

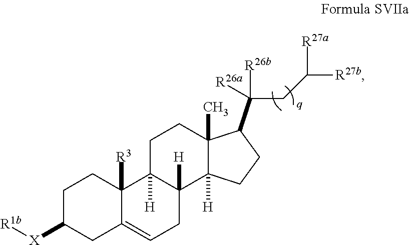

or a pharmaceutically acceptable salt thereof.

In some embodiments, the compound has the structure of Formula SVIIb:

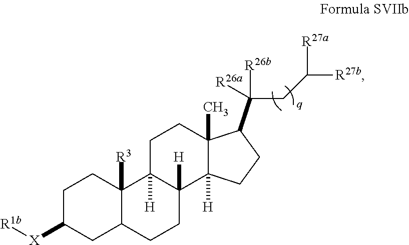

or a pharmaceutically acceptable salt thereof.

In some embodiments, R^{26a} and R^{26b} is, independently, H,

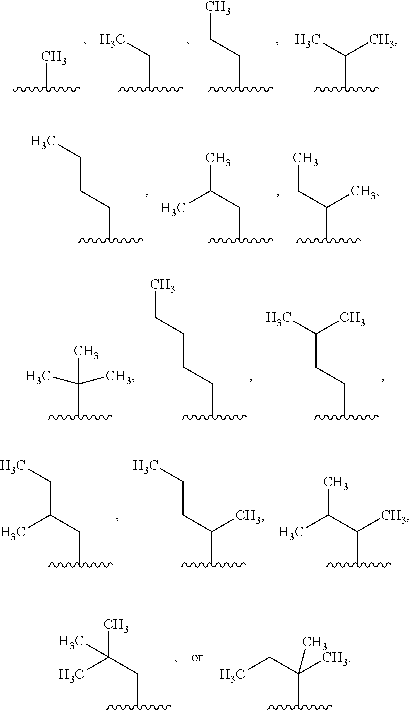

In some embodiments, R^{26a} and R^{26b}, together with the atom to which each is attached, combine to form

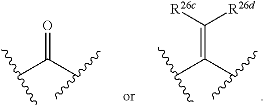

In some embodiments, R^{26a} and R^{26b}, together with the atom to which each is attached, combine to form

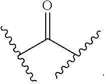 [SMILES: *C(*)=O]

In some embodiments, R^{26a} and R^{26b}, together with the atom to which each is attached, combine to form

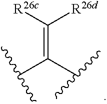

In some embodiments, where each of R^{26c} and R²⁶ is, independently, H,

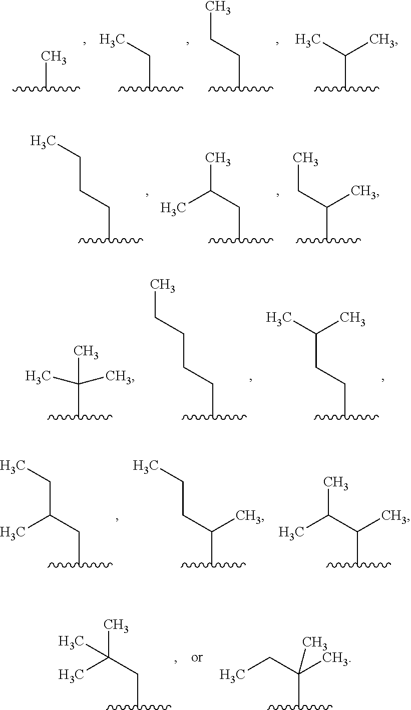

In some embodiments, each of R^{27a} and R^{27b} is H, hydroxyl, or optionally substituted C₁-C₃ alkyl.

In some embodiments, each of R^{27a} and R^{27b} is, independently, H, hydroxyl,

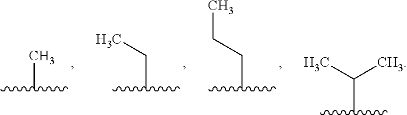

In an aspect, the structural lipid of the invention features a compound having the structure of Formula SVIII:

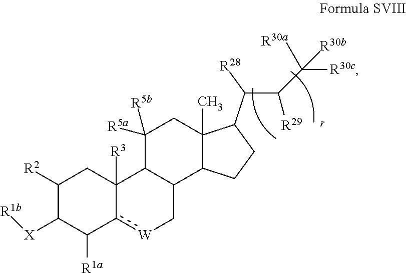

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

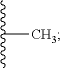

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

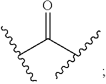 [SMILES: *C(*)=O]

R²⁸ is H or optionally substituted C₁-C₆ alkyl;

r is 1, 2, or 3;

each R²⁹ is, independently, H or optionally substituted C₁-C₆ alkyl; and

each of R^{30a}, R^{30b}, and R^{30c} is C₁-C₆ alkyl,

or a pharmaceutically acceptable salt thereof.

In some embodiments, the compound has the structure of Formula SVIIIa:

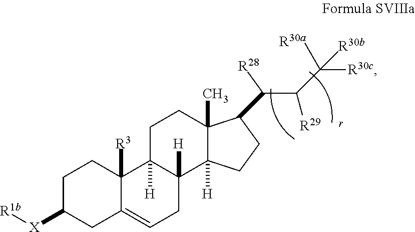

or a pharmaceutically acceptable salt thereof.

In some embodiments, the compound has the structure of Formula SVIIIb:

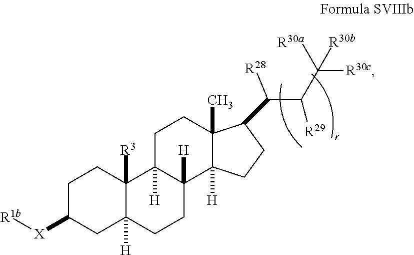

or a pharmaceutically acceptable salt thereof.

In some embodiments, R²⁸ is H,

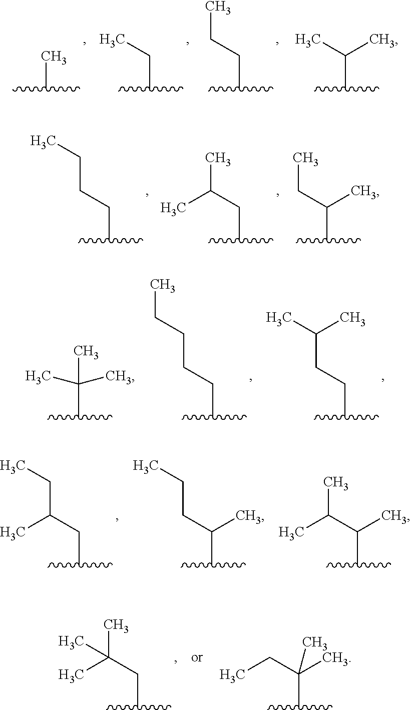

In some embodiments, R²⁸ is

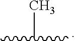

In some embodiments, each of R^{30a}, R^{30b}, and R^{30c} is, independently,

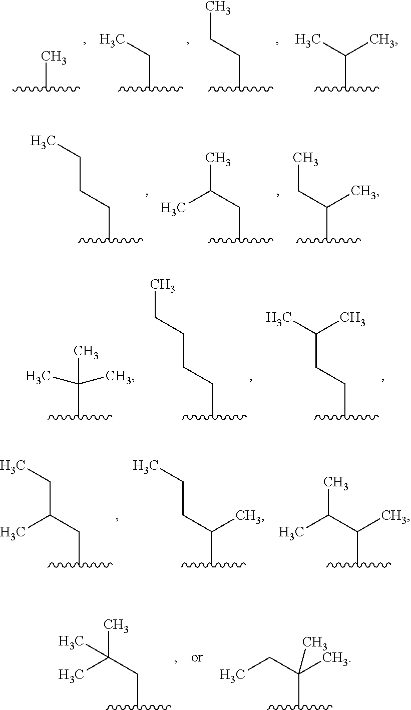

In some embodiments, r is 1. In some embodiments, r is 2. In some embodiments, r is 3.

In some embodiments, each R²⁹ is, independently, H,

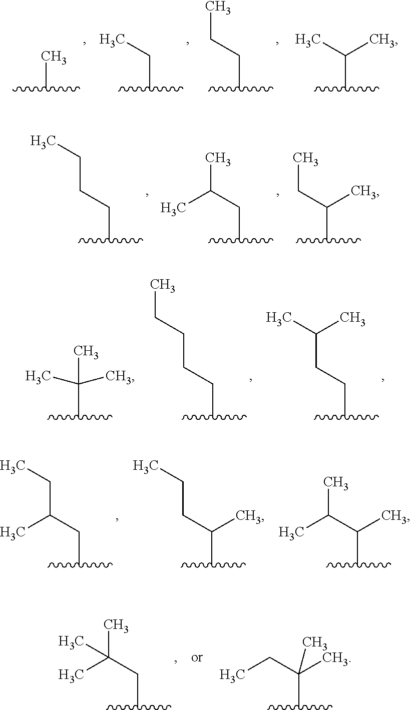

In some embodiments, each R²⁹ is, independently, H or

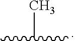

In an aspect, the structural lipid of the invention features a compound having the structure of Formula SIX:

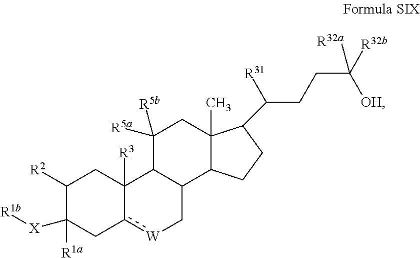

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

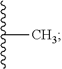

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

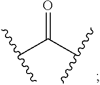 [SMILES: *C(*)=O]

R³¹ is H or C₁-C₆ alkyl; and

each of R^{32a} and R^{32b} is C₁-C₆ alkyl,

or a pharmaceutically acceptable salt thereof.

In some embodiments, the compound has the structure of Formula SIXa:

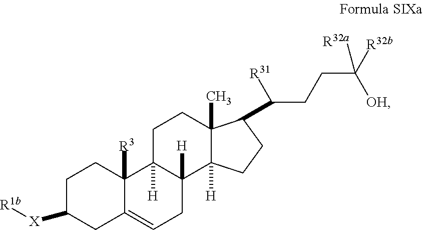

or a pharmaceutically acceptable salt thereof.

In some embodiments, the compound has the structure of Formula SIXb:

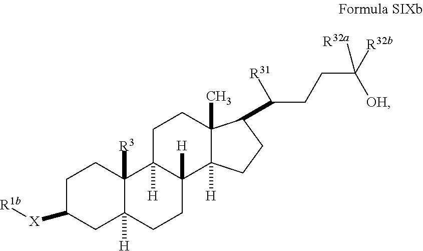

or a pharmaceutically acceptable salt thereof.

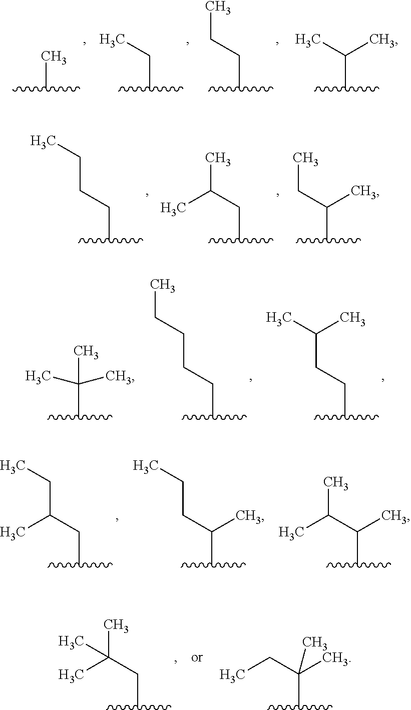

In some embodiments, R³¹ is H,

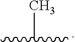

In some embodiments, R³¹ is

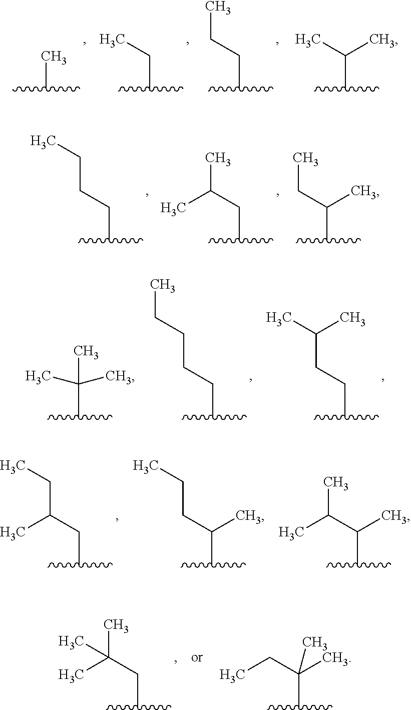

In some embodiments, each of R^{32a} and R^{32b} is, independently,

In an aspect, the structural lipid of the invention features a compound having the structure of Formula SX:

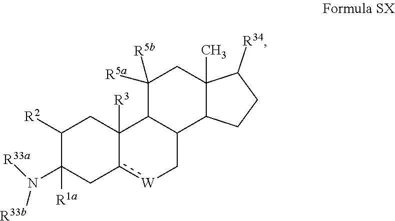

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

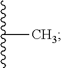

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

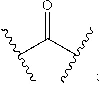 [SMILES: *C(*)=O]

R^{33a} is optionally substituted C₁-C₆ alkyl or

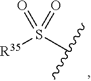

where R³⁵ is optionally substituted C₁-C₆ alkyl or optionally substituted C₆-C₁₀ aryl;

R^{33b} is H or optionally substituted C₁-C₆ alkyl; or

R³⁵ and R^{33b}, together with the atom to which each is attached, form an optionally substituted C₃-C₉ heterocyclyl; and

R³⁴ is optionally substituted C₁-C₆ alkyl or optionally substituted C₁-C₆ heteroalkyl,

or a pharmaceutically acceptable salt thereof.

In some embodiments, the compound has the structure of Formula SXa:

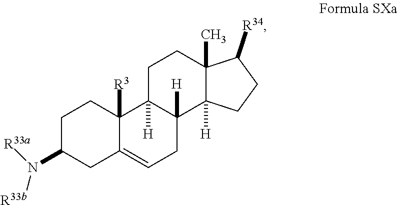

or a pharmaceutically acceptable salt thereof.

In some embodiments, the compound has the structure of Formula SXb:

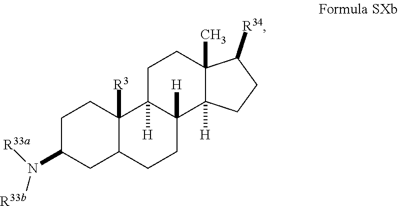

or a pharmaceutically acceptable salt thereof.

In some embodiments, R^{33a} is

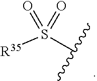

In some embodiments, R³⁵ is

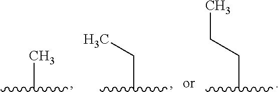

In some embodiments, R³⁵ is

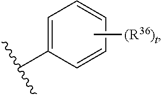

where

t is 0, 1, 2, 3, 4, or 5; and

each R³⁶ is, independently, halo, hydroxyl, optionally substituted C₁-C₆ alkyl, or optionally substituted C₁-C₆ heteroalkyl.

In some embodiments, R³⁴ is

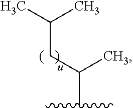

where u is 0, 1, 2, 3, or 4.

In some embodiments, u is 3 or 4.

In an aspect, the structural lipid of the invention features a compound having the structure of Formula SXI:

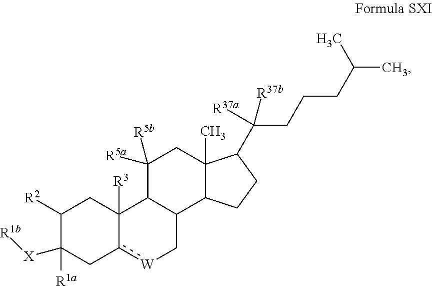

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

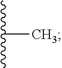

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

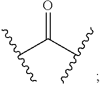 [SMILES: *C(*)=O]

and

each of R^{37a} and R^{37b} is, independently, optionally substituted C₁-C₆ alkyl, optionally substituted C₁-C₆ heteroalkyl, halo, or hydroxyl,

or a pharmaceutically acceptable salt thereof.

In some embodiments, the compound has the structure of Formula SXIa:

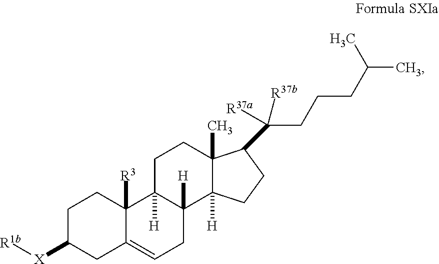

or a pharmaceutically acceptable salt thereof.

In some embodiments, the compound has the structure of Formula SXIb:

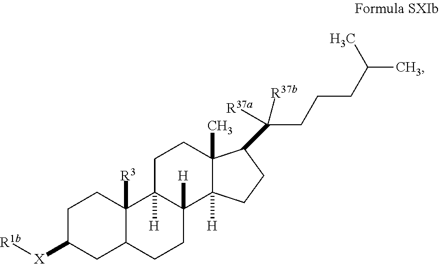

or a pharmaceutically acceptable salt thereof.

In some embodiments, R^{37a} is hydroxyl.

In some embodiments, R^{37b} is

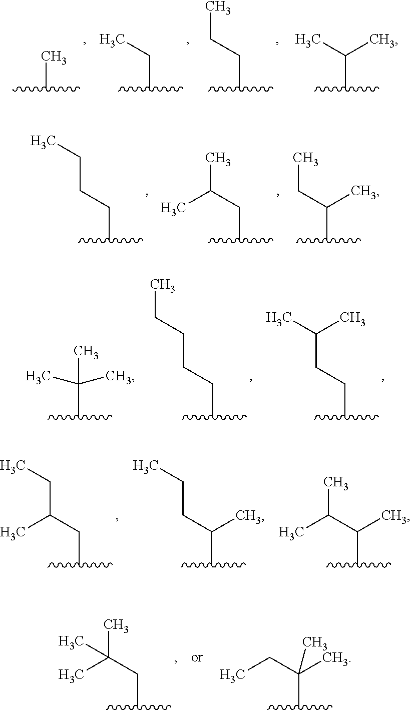

In an aspect, the structural lipid of the invention features a compound having the structure of Formula SXII:

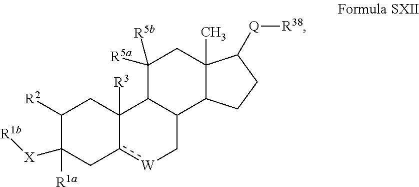

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

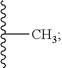

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

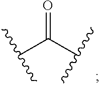 [SMILES: *C(*)=O]

and

Q is O, S, or NR^{E}, where R^{E} is H or optionally substituted C₁-C₆ alkyl; and

R³⁸ is optionally substituted C₁-C₆ alkyl,

or a pharmaceutically acceptable salt thereof.

In some embodiments, the compound has the structure of Formula SXIIa:

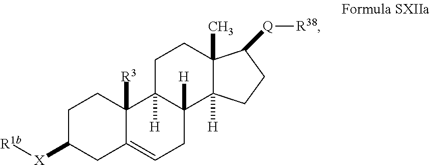

or a pharmaceutically acceptable salt thereof.

In some embodiments, the compound has the structure of Formula SXIIb:

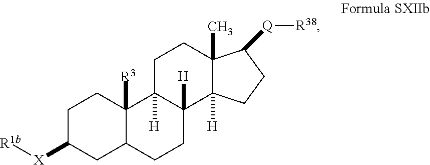

or a pharmaceutically acceptable salt thereof.

In some embodiments, Q is NR^{E}.

In some embodiments, R^{E} is H or

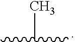

In some embodiments, RE is H. In some embodiments, RE is

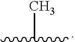

In some embodiments, R³⁸ is

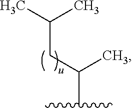

where u is 0, 1, 2, 3, or 4.

In some embodiments, X is O.

In some embodiments, R^{1a} is H or optionally substituted C₁-C₆ alkyl.

In some embodiments, R^{1a} is H.

In some embodiments, R^{1b} is H or optionally substituted C₁-C₆ alkyl.

In some embodiments, R^{1b} is H.

In some embodiments, R² is H.

In some embodiments, R^{4a} is H.

In some embodiments, R^{4b} is H.

In some embodiments, [symbol] represents a double bond.

In some embodiments, R³ is H. In some embodiments, R³ is

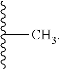

In some embodiments, R^{5a} is H.

In some embodiments, R^{5b} is H.

In an aspect, the invention features a compound having the structure of any one of compounds S-1-42, S-150, S-154, S-162-165, S-169-172 and S-184 in Table 1, or any pharmaceutically acceptable salt thereof. As used herein, “CMPD” refers to “compound.”

TABLE 1
Compounds of Formula SI

| CMPD No. S- | Structure |
|---|---|
| 1 | 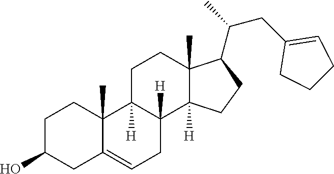 |
| 2 | 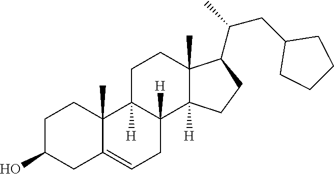 |
| 3 | 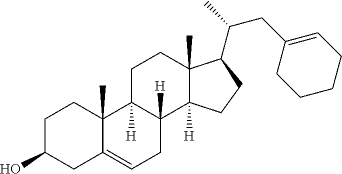 |
| 4 | 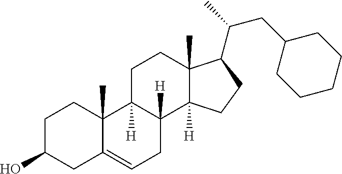 |
| 5 | 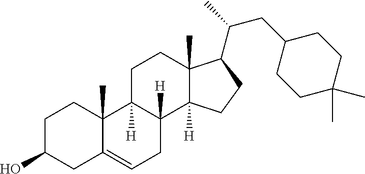 |
| 6 | 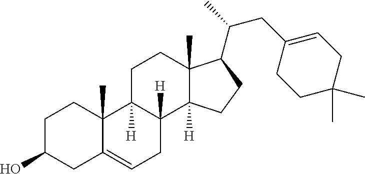 |
| 7 | 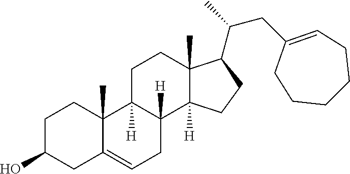 |
| 8 | 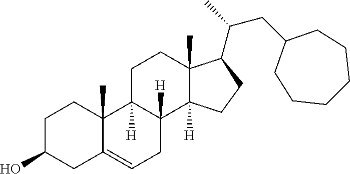 |
| 9 | 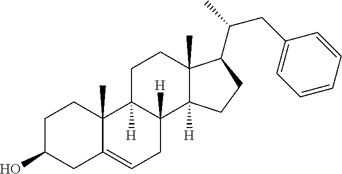 |
| 10 | 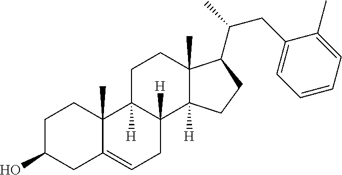 |
| 11 | 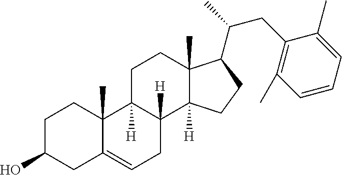 |
| 12 | 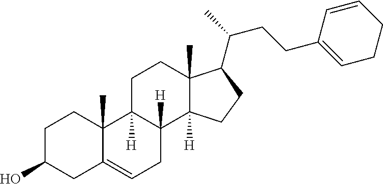 |
| 13 | 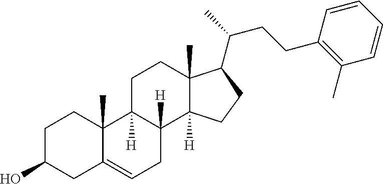 |
| 14 | 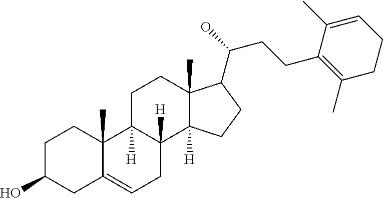 |
| 15 | 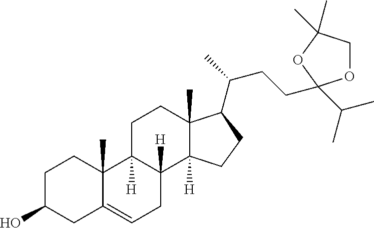 |
| 16 | 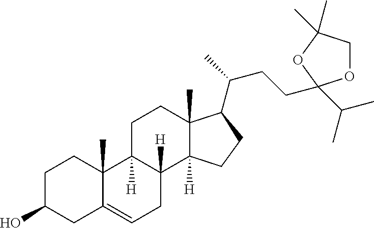 |
| 17 | 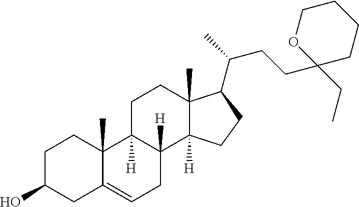 |
| 18 | 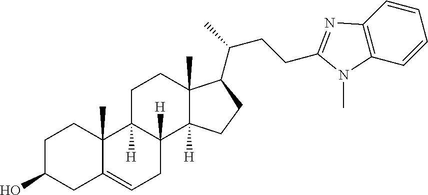 |
| 19 | 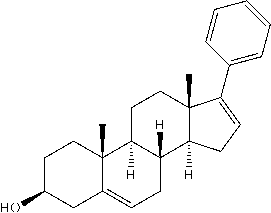 |
| 20 | 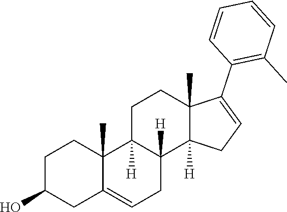 |
| 21 | 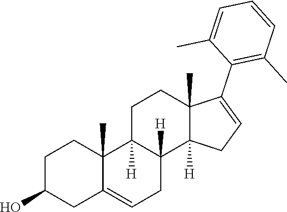 |
| 150 | 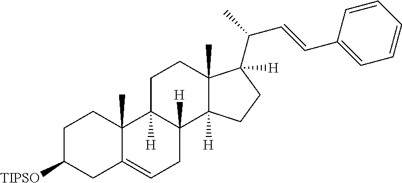 |
| 154 | 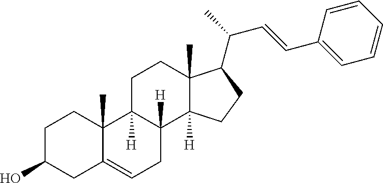 |
| 162 | 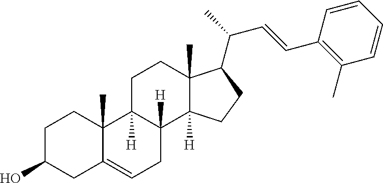 |
| 163 | 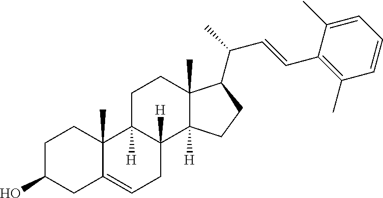 |
| 164 | 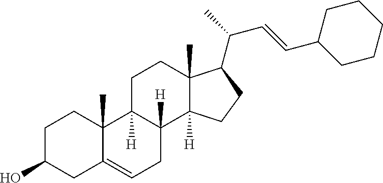 |
| 184 | 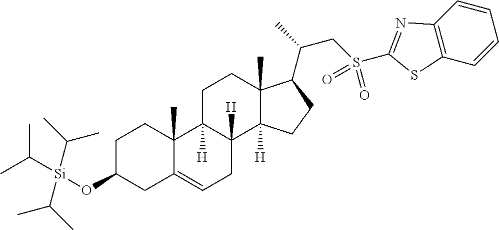 |
| 22 | 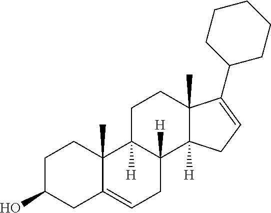 |
| 23 | 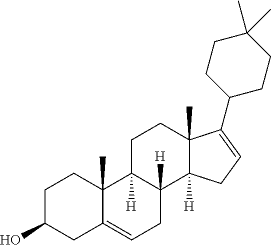 |
| 24 | 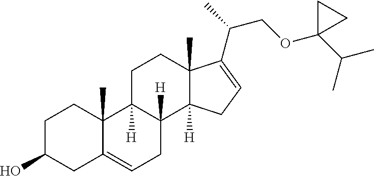 |
| 25 | 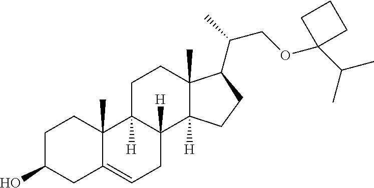 |
| 26 | 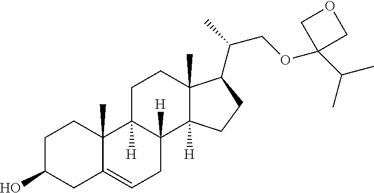 |
| 27 | 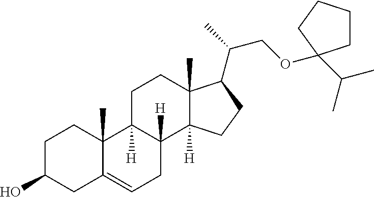 |
| 28 | 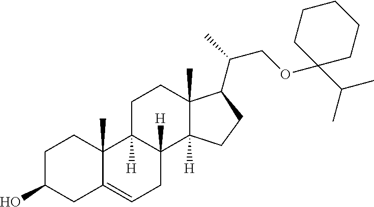 |
| 29 | 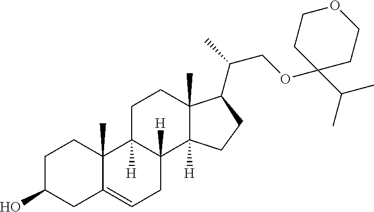 |
| 30 | 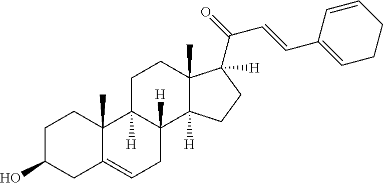 |
| 31 | 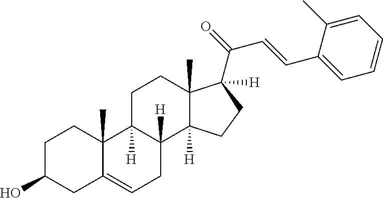 |
| 32 | 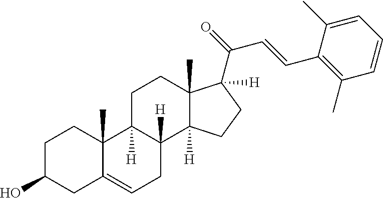 |
| 33 | 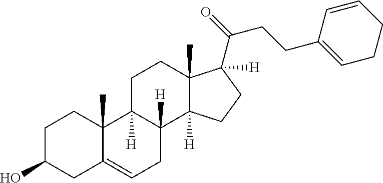 |
| 34 | 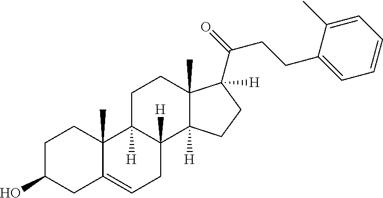 |
| 35 | 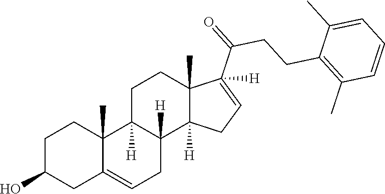 |
| 36 | 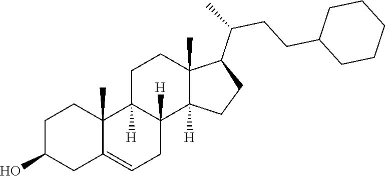 |
| 37 | 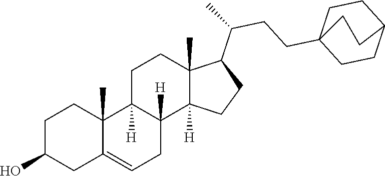 |
| 38 | 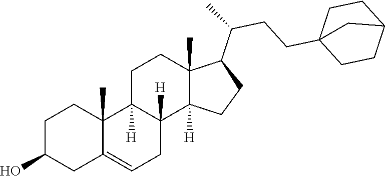 |
| 39 | 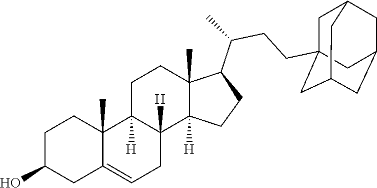 |
| 40 | 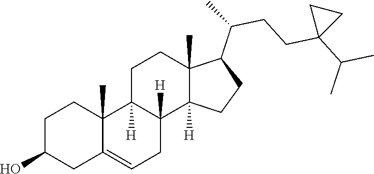 |
| 41 | 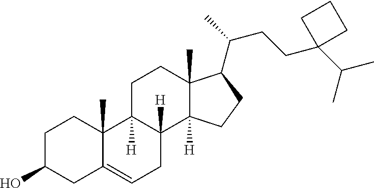 |
| 42 | 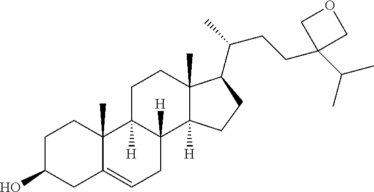 |
| 165 | 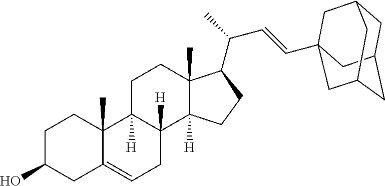 |
| 169 | 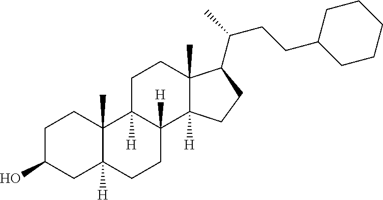 |
| 170 | 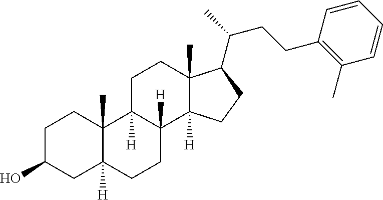 |
| 171 | 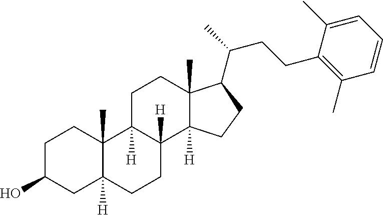 |
| 172 | 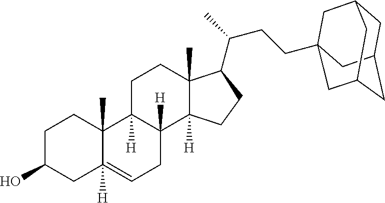 |

In an aspect, the invention features a compound having the structure of any one of compounds S-43-50 and S-175-178 in Table 2, or any pharmaceutically acceptable salt thereof.

TABLE 2
Compounds of Formula SII

| CMPD No. S- | Structure |
|---|---|
| 43 | 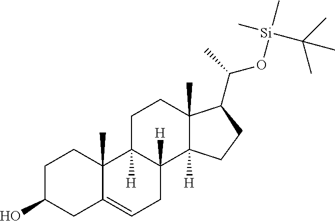 |
| 44 | 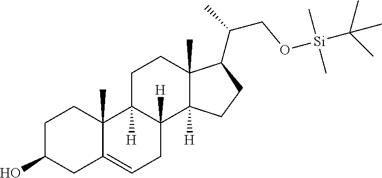 |
| 45 | 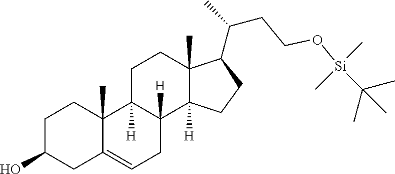 |
| 46 | 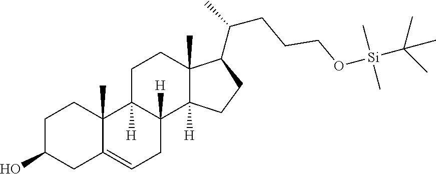 |
| 175 | 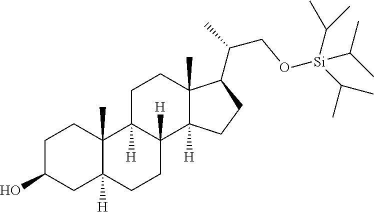 |
| 176 | 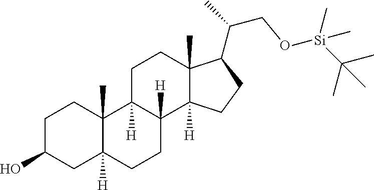 |
| 47 | 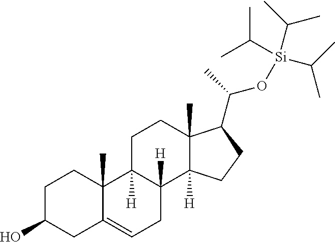 |
| 48 | 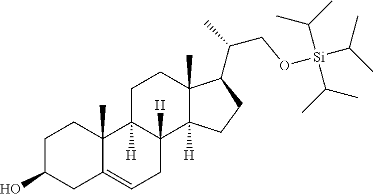 |
| 49 | 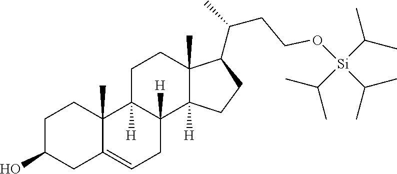 |
| 50 | 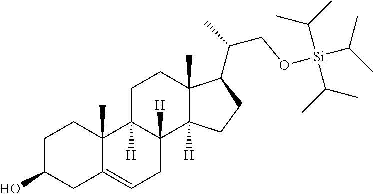 |
| 177 | 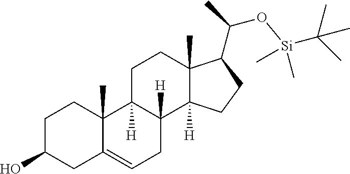 |
| 178 | 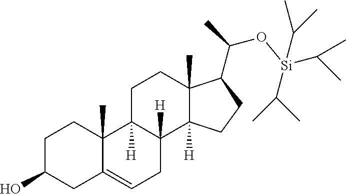 |

In an aspect, the invention features a compound having the structure of any one of compounds S-51-67, S-149 and S-153 in Table 3, or any pharmaceutically acceptable salt thereof.

TABLE 3
Compounds of Formula SIII

| CMPD No. S- | Structure |
|---|---|
| 51 | 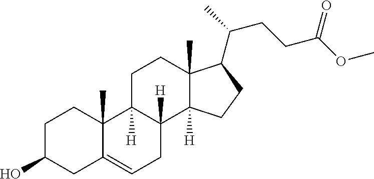 |
| 52 | 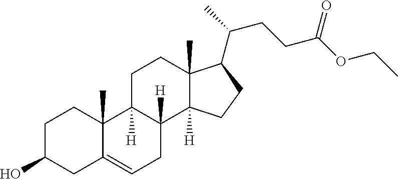 |
| 53 | 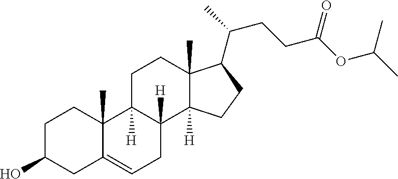 |
| 54 | 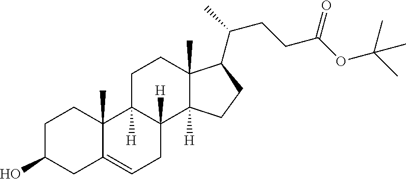 |
| 55 | 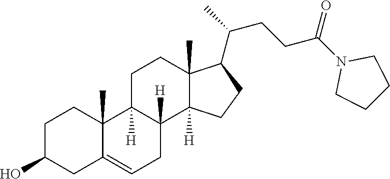 |
| 56 | 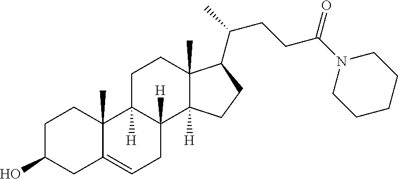 |
| 57 | 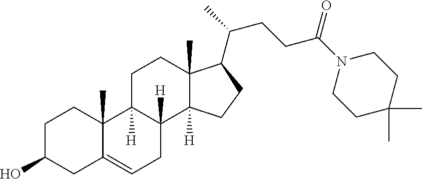 |
| 58 | 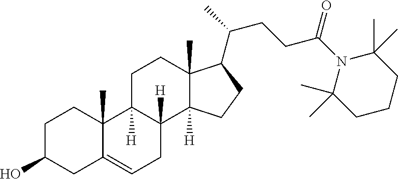 |
| 59 | 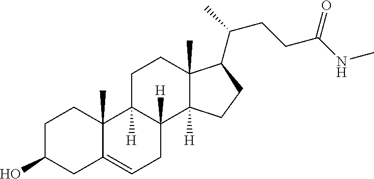 |
| 153 | 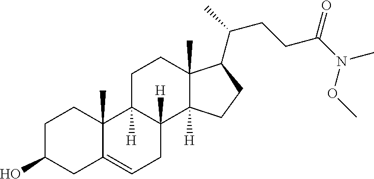 |
| 60 | 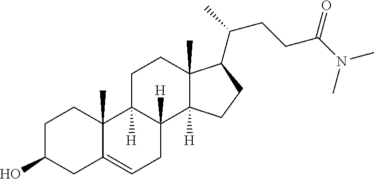 |
| 61 | 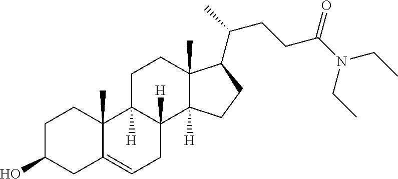 |
| 62 | 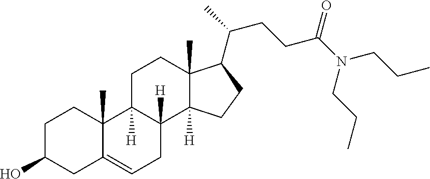 |
| 63 | 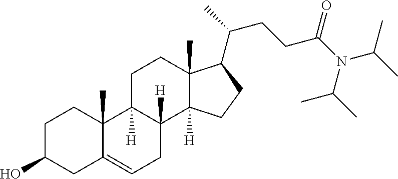 |
| 64 | 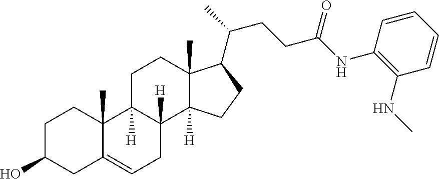 |
| 65 | 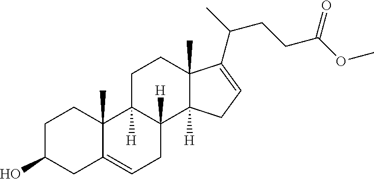 |
| 66 | 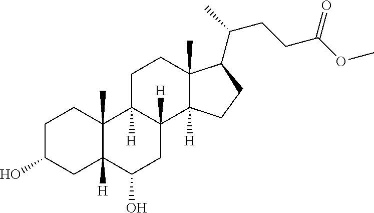 |
| 67 | 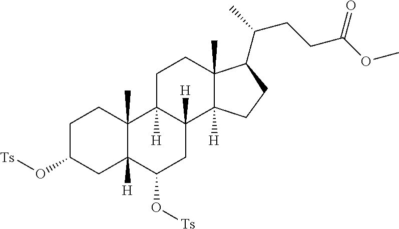 |
| 149 | 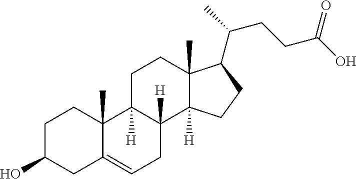 |

In an aspect, the invention features a compound having the structure of any one of compounds S-68-73 in Table 4, or any pharmaceutically acceptable salt thereof.

TABLE 4
Compounds of Formula SIV

| CMPD No. S- | Structure |
|---|---|
| 68 | 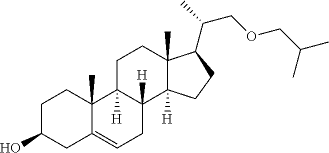 |
| 69 | 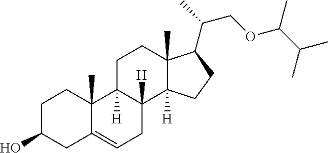 |
| 70 | 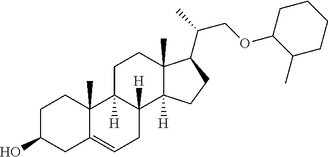 |
| 71 | 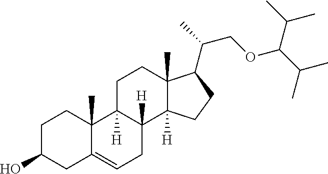 |
| 72 | 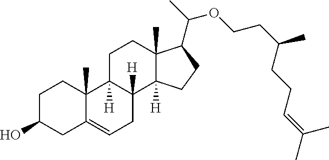 |
| 73 | 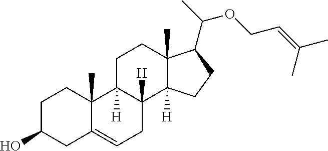 |

In an aspect, the invention features a compound having the structure of any one of compounds S-74-78 in Table 5, or any pharmaceutically acceptable salt thereof.

TABLE 5
Compounds of Formula SV

| CMPD No. S- | Structure |
|---|---|
| 74 | 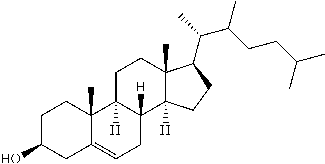 |
| 75 | 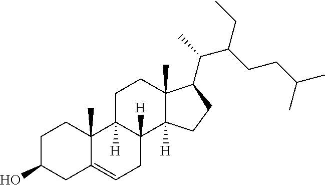 |
| 76 | 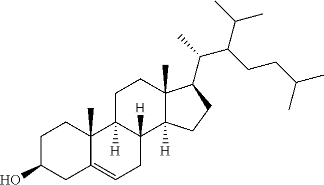 |
| 77 | 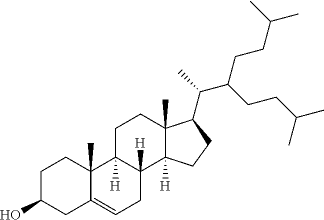 |
| 78 | 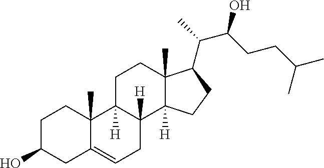 |

In an aspect, the invention features a compound having the structure of any one of compounds S-79 or S-80 in Table 6, or any pharmaceutically acceptable salt thereof.

TABLE 6
Compounds of Formula SVI

| CMPD No. S- | Structure |
|---|---|
| 79 | 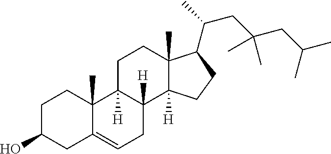 |
| 80 | 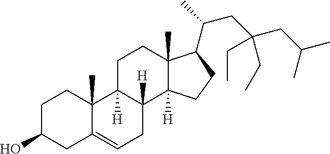 |

In an aspect, the invention features a compound having the structure of any one of compounds S-81-87, S-152 and S-157 in Table 7, or any pharmaceutically acceptable salt thereof.

TABLE 7
Compounds of Formula S-VII

| CMPD No. S- | Structure |
|---|---|
| 81 | 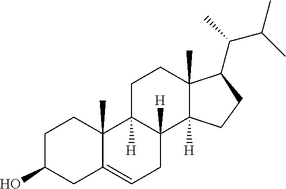 |
| 82 | 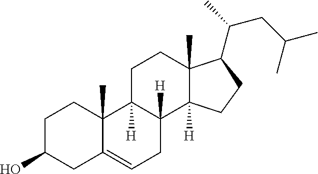 |
| 83 | 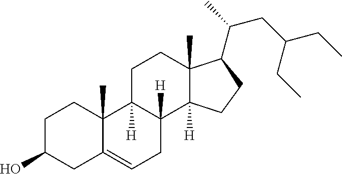 |
| 84 | 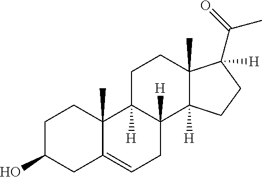 |
| 157 | 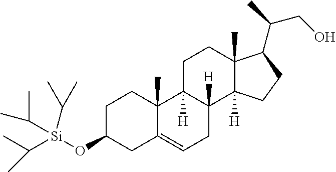 |
| 85 | 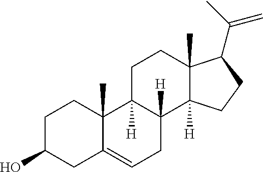 |
| 86 | 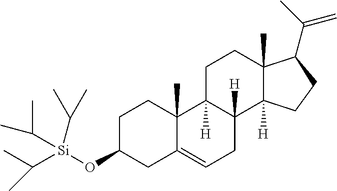 |
| 87 | 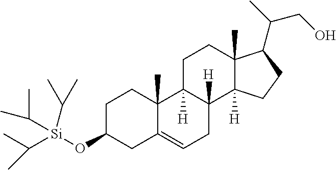 |
| 152 | 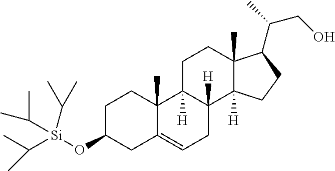 |

In an aspect, the invention features a compound having the structure of any one of compounds S-88-97 in Table 8, or any pharmaceutically acceptable salt thereof.

TABLE 8
Compounds of Formula SVIII

| CMPD No. S- | Structure |
|---|---|
| 88 | 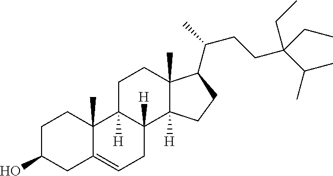 |
| 89 | 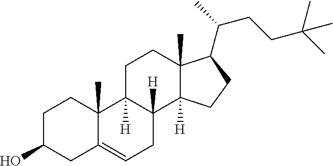 |
| 90 | 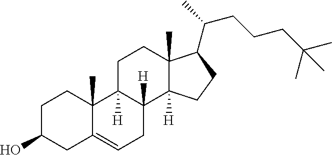 |
| 91 | 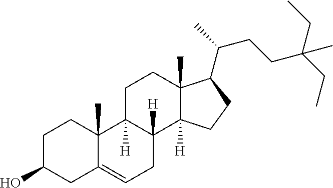 |
| 92 | 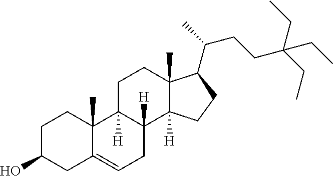 |
| 93 | 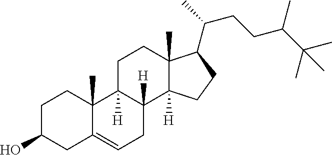 |
| 94 | 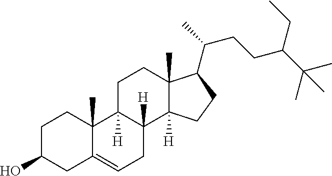 |
| 95 | 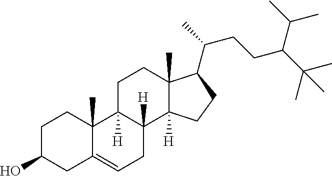 |
| 96 | 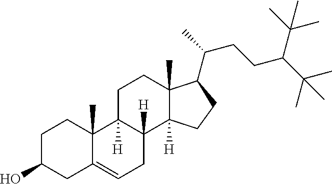 |
| 97 | 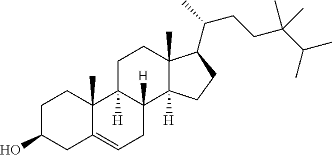 |

In an aspect, the invention features a compound having the structure of any one of compounds S-98-105 and S-180-182 in Table 9, or any pharmaceutically acceptable salt thereof.

TABLE 9
Compounds of Formula SIX

| CMPD No. S- | Structure |
|---|---|
| 98 | 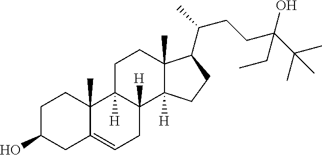 |
| 99 | 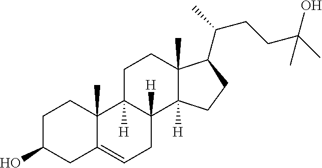 |
| 100 | 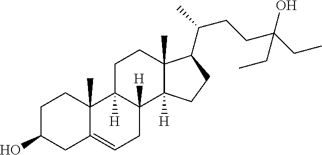 |
| 101 | 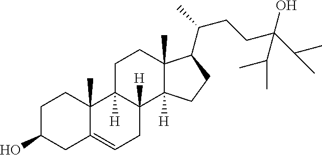 |
| 180 | 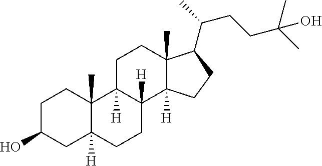 |
| 181 | 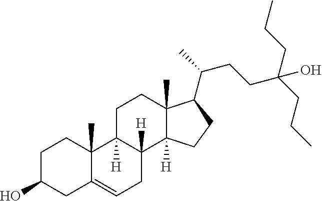 |
| 102 | 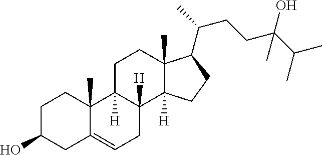 |
| 103 | 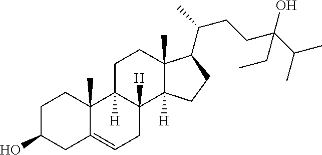 |
| 104 | 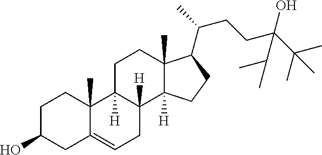 |
| 105 | 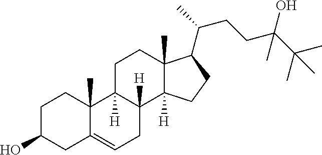 |
| 182 | 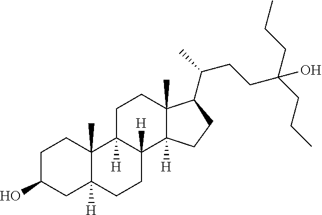 |

In an aspect, the invention features a compound having the structure of compound S-106 in Table 10, or any pharmaceutically acceptable salt thereof.

TABLE 10
Compounds of Formula SX

| CMPD No. S- | Structure |
|---|---|
| 106 | 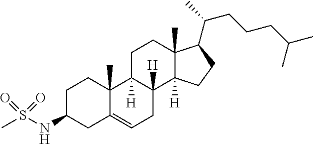 |

In an aspect, the invention features a compound having the structure of compound S-107 or S-108 in Table 11, or any pharmaceutically acceptable salt thereof.

TABLE 11
Compounds of Formula SXI

| CMPD No. S- | Structure |
|---|---|
| 107 | 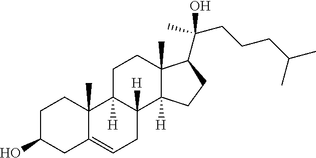 |
| 108 | 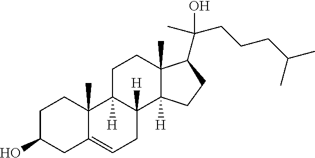 |

In an aspect, the invention features a compound having the structure of compound S-109 in Table 12, or any pharmaceutically acceptable salt thereof.

TABLE 12
Compounds of Formula SXII

| CMPD No. S- | Structure |
|---|---|
| 109 | 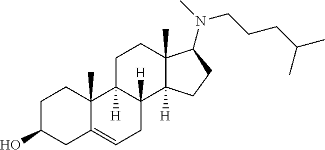 |

In an aspect, the invention features a compound having the structure of any one of compounds S-110-130, S-155, S-156, S-158, S-160, S-161, S-166-168, S-173, S-174 and S-179 in Table 13, or any pharmaceutically acceptable salt thereof.

TABLE 13
Compounds of the Invention

| CMPD No. S- | Structure |
|---|---|
| 110 | 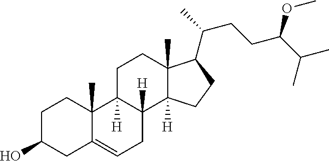 |
| 111 | 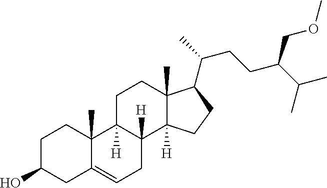 |
| 112 | 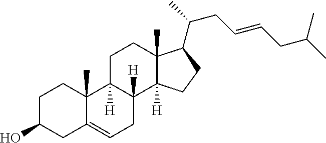 |
| 113 | 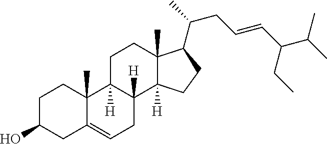 |
| 114 | 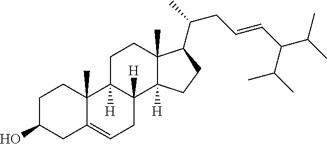 |
| 115 | 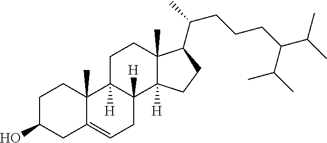 |
| 116 | 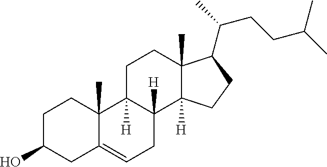 |
| 117 | 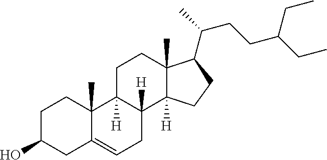 |
| 118 | 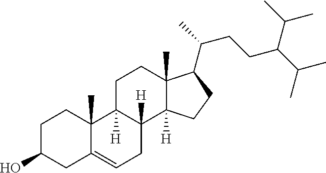 |
| 119 | 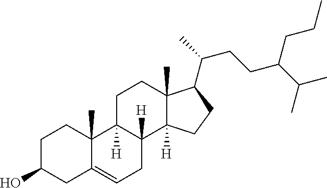 |
| 120 | 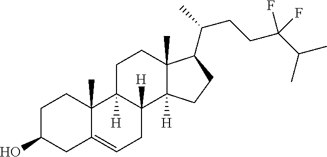 |
| 156 | 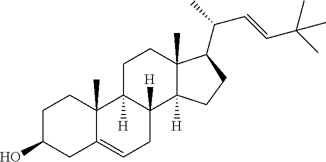 |
| 158 | 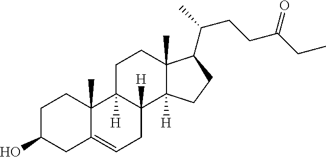 |
| 160 | 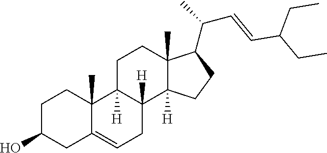 |
| 161 | 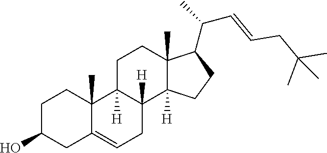 |
| 166 | 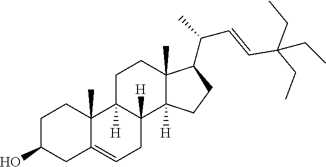 |
| 121 | 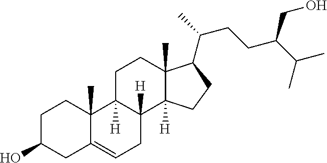 |
| 122 | 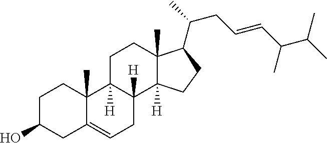 |
| 123 | 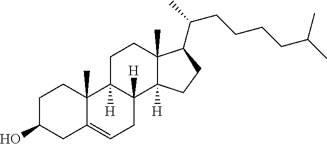 |
| 124 | 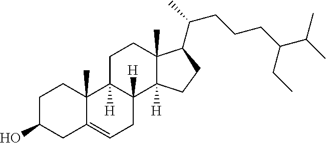 |
| 125 | 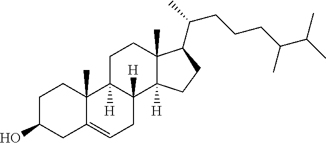 |
| 126 | 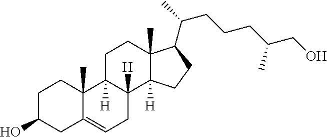 |
| 127 | 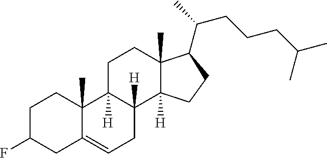 |
| 128 | 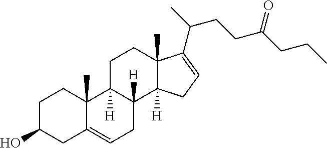 |
| 129 | 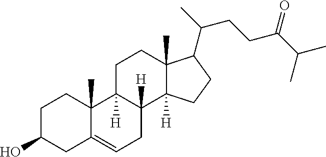 |
| 130 | 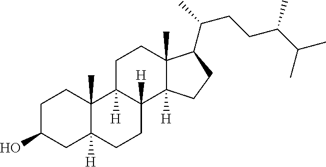 |
| 155 | 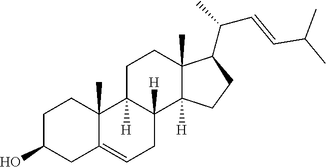 |
| 167 | 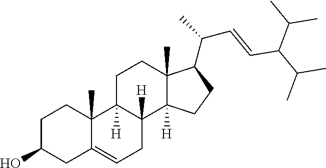 |
| 168 | 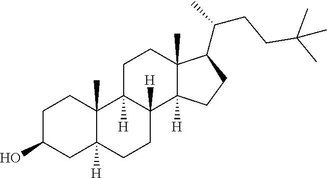 |
| 173 | 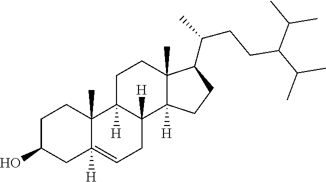 |
| 174 | 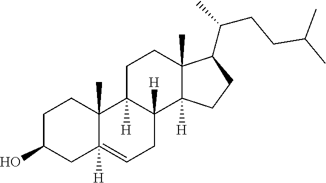 |
| 179 | 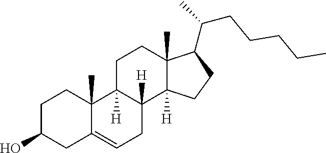 |

In an aspect, the invention features a compound having the structure of any one of compounds S-131-133 in Table 14, or any pharmaceutically acceptable salt thereof.

TABLE 14
Compounds of the Invention

| CMPD No. S- | Structure |
|---|---|
| 131 | 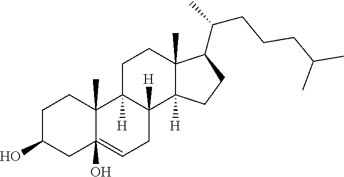 |
| 132 | 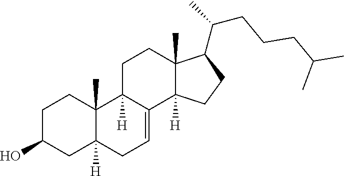 |
| 133 | 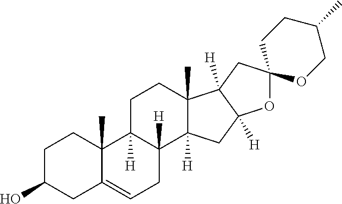 |

In an aspect, the invention features a compound having the structure of any one of compounds S-134-148, S-151 and S-159 in Table 15, or any pharmaceutically acceptable salt thereof.

TABLE 15
Compounds of the Invention

| CMPD No. S- | Structure |
|---|---|
| 134 | 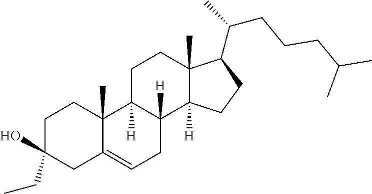 |
| 135 | 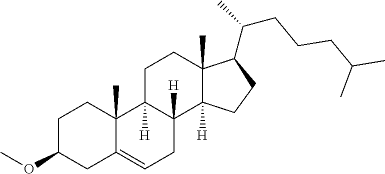 |
| 136 | 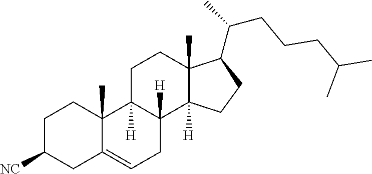 |
| 137 | 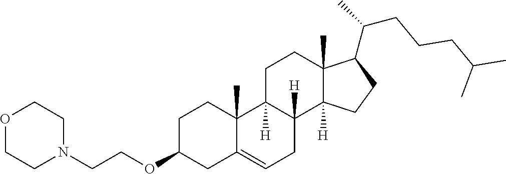 |
| 138 | 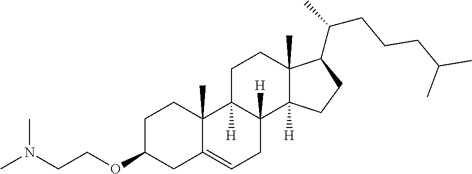 |
| 139 | 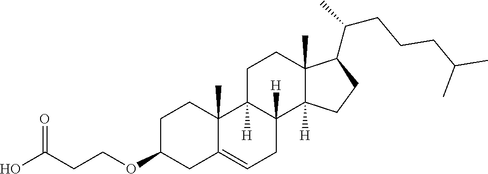 |
| 140 | 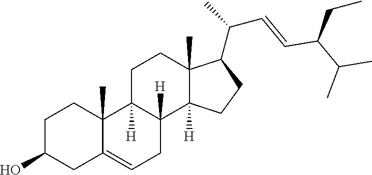 |
| 141 | 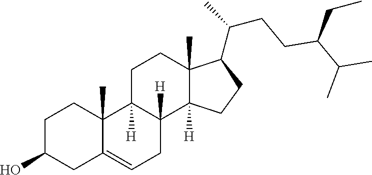 |
| 159 | 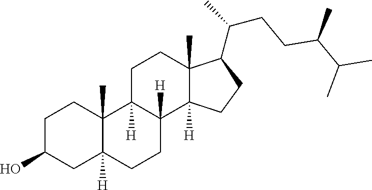 |
| 142 | 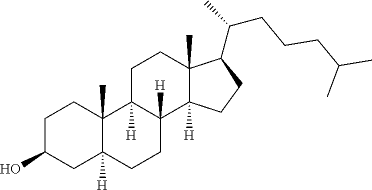 |
| 143 | 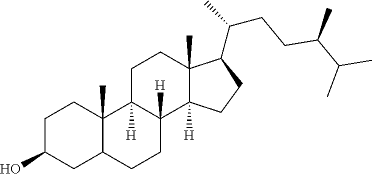 |
| 144 | 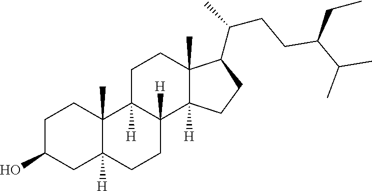 |
| 145 | 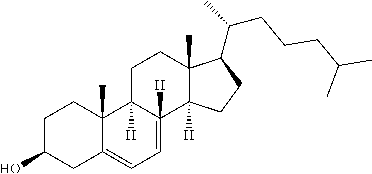 |
| 146 | 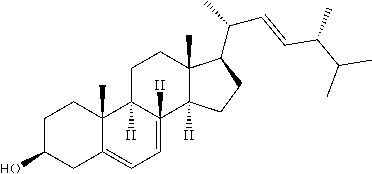 |
| 147 | 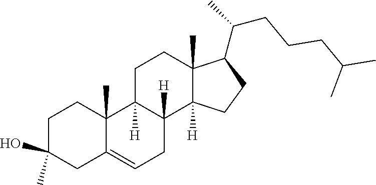 |
| 148 | 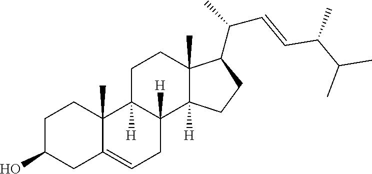 |
| 151 | 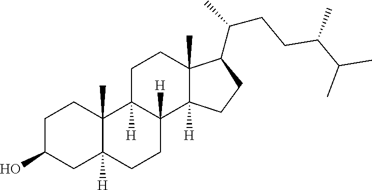 |

The one or more structural lipids of the lipid nanoparticles of the invention can be a composition of structural lipids (e.g., a mixture of two or more structural lipids, a mixture of three or more structural lipids, a mixture of four or more structural lipids, or a mixture of five or more structural lipids). A composition of structural lipids can include, but is not limited to, any combination of sterols (e.g., cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds 134-148, 151, and 159 in Table 15). For example, the one or more structural lipids of the lipid nanoparticles of the invention can be composition 183 in Table 16.

TABLE 16
Structural Lipid Compositions

| Composition | | |
|---|---|---|
| S- No. | Structure | |
| 183 | 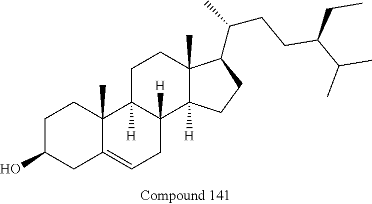 | 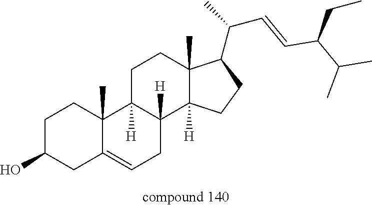 |
| | 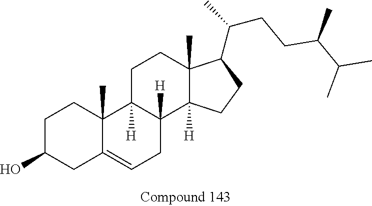 | 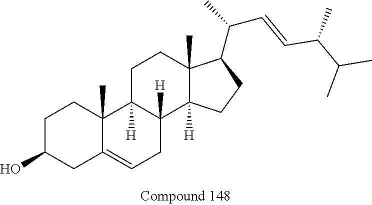 |

Composition S-183 is a mixture of compounds S-141, S-140, S-143, and S-148. In some embodiments, composition S-183 includes about 35% to about 45% of compound S-141, about 20% to about 30% of compound S-140, about 20% to about 30% compound S-143, and about 5% to about 15% of compound S-148. In some embodiments, composition 183 includes about 40% of compound S-141, about 25% of compound S-140, about 25% compound S-143, and about 10% of compound S-148.

In some embodiments, the structural lipid is a pytosterol. In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

Ratio of Compounds

A lipid nanoparticle of the invention can include a structural component as described herein. The structural component of the lipid nanoparticle can be any one of compounds S-1-148, a mixture of one or more structural compounds of the invention and/or any one of compounds S-1-148 combined with a cholesterol and/or a phytosterol.

For example, the structural component of the lipid nanoparticle can be a mixture of one or more structural compounds (e.g. any of Compounds 5-1-148) of the invention with cholesterol. The mol % of the structural compound present in the lipid nanoparticle relative to cholesterol can be from 0-99 mol %. The mol % of the structural compound present in the lipid nanoparticle relative to cholesterol can be about 10 mol %, 20 mol %, 30 mol %, 40 mol %, 50 mol %, 60 mol %, 70 mol %, 80 mol %, or 90 mol %.

In one aspect, the invention features a composition including two or more sterols, wherein the two or more sterols include at least two of: β-sitosterol, sitostanol, camesterol, stigmasterol, and brassicasteol. The composition may additionally comprise cholesterol. In one embodiment, β-sitosterol comprises about 35-99%, e.g., about 40%, 50%, 60%, 70%, 80%, 90%, 95% or greater of the non-cholesterol sterol in the composition.

In another aspect, the invention features a composition including two or more sterols, wherein the two or more sterols include β-sitosterol and campesterol, wherein β-sitosterol includes 95-99.9% of the sterols in the composition and campesterol includes 0.1-5% of the sterols in the composition.

In some embodiments, the composition further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including two or more sterols, wherein the two or more sterols include β-sitosterol and sitostanol, wherein β-sitosterol includes 95-99.9% of the sterols in the composition and sitostanol includes 0.1-5% of the sterols in the composition.

In some embodiments, the composition further includes campesterol. In some embodiments, β-sitosterol includes 95-99.9%, campesterol includes 0.05-4.95%, and sitostanol includes 0.05-4.95% of the sterols in the composition.

In some embodiments, the composition further includes campesterol. In some embodiments, β-sitosterol includes 75-80%, campesterol includes 5-10%, and sitostanol includes 10-15% of the sterols in the composition.

In some embodiments, the composition further includes an additional sterol. In some embodiments, β-sitosterol includes 35-45%, stigmasterol includes 20-30%, and campesterol includes 20-30%, and brassicasterol includes 1-5% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and campesterol and β-sitosterol includes 95-99.9% of the sterols in the composition and campesterol includes 0.1-5% of the sterols in the composition.

In some embodiments, the two or more sterols further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and sitostanol and β-sitosterol includes 95-99.9% of the sterols in the composition and sitostanol includes 0.1-5% of the sterols in the composition.

In some embodiments, the two or more sterols further includes campesterol. In some embodiments, β-sitosterol includes 95-99.9%, campesterol includes 0.05-4.95%, and sitostanol includes 0.05-4.95% of the sterols in the composition.

(iii) Non-Cationic Helper Lipids/Phospholipids

In some embodiments, the lipid-based composition (e.g., LNP) described herein comprises one or more non-cationic helper lipids. In some embodiments, the non-cationic helper lipid is a phospholipid. In some embodiments, the non-cationic helper lipid is a phospholipid substitute or replacement.

As used herein, the term “non-cationic helper lipid” refers to a lipid comprising at least one fatty acid chain of at least 8 carbons in length and at least one polar head group moiety. In one embodiment, the helper lipid is not a phosphatidyl choline (PC). In one embodiment the non-cationic helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-i77-glycero-3-phosphocholine (DSPC) substitute.

Phospholipids

The lipid composition of the pharmaceutical composition disclosed herein can comprise one or more non-cationic helper lipids. In some embodiments, the non-cationic helper lipids are phospholipids, for example, one or more saturated or (poly)unsaturated phospholipids or a combination thereof. In general, phospholipids comprise a phospholipid moiety and one or more fatty acid moieties. As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

The lipid component of a lipid nanoparticle of the disclosure may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to Formula (H III):

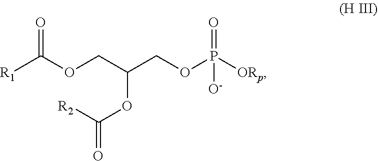

in which Rₚ represents a phospholipid moiety and R₁ and R₂ represent fatty acid moieties with or without unsaturation that may be the same or different. A phospholipid moiety may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a LNP to facilitate membrane permeation or cellular recognition or in conjugating a LNP to a useful component such as a targeting or imaging moiety (e.g., a dye). Each possibility represents a separate embodiment of the present invention.

Phospholipids useful in the compositions and methods described herein may be selected from the non-limiting group consisting of
1,2-distearoyl-sn-glycero-3-phosphocholine (DSPC),
1,2-dioleoyl-sn-glycero-3-phosphoethanolamine (DOPE),
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine (18:3 (cis) PC),
1,2-diarachidonoyl-sn-glycero-3-phosphocholine (DAPC),
1,2-didocosahexaenoyl-sn-glycero-3-phosphocholine(22:6 (cis) PC)
1,2-diphytanoyl-sn-glycero-3-phosphoethanolamine (4ME 16.0 PE),
1,2-distearoyl-sn-glycero-3-phosphoethanolamine (DSPE),
1,2-dilinoleoyl-sn-glycero-3-phosphoethanolamine (PE(18:2/18:2),
1,2-dilinolenoyl-sn-glycero-3-phosphoethanolamine (PE 18:3(9Z, 12Z, 15Z),
1,2-diarachidonoyl-sn-glycero-3-phosphoethanolamine (DAPE 18:3 (9Z, 12Z, 15Z),
1,2-didocosahexaenoyl-sn-glycero-3-phosphoethanolamine (22:6 (cis) PE),
1,2-dioleoyl-sn-glycero-3-phospho-rac-(1-glycerol) sodium salt (DOPG),
and sphingomyelin. Each possibility represents a separate embodiment of the invention.

In some embodiments, a LNP includes DSPC. In certain embodiments, a LNP includes DOPE. In some embodiments, a LNP includes DMPE. In some embodiments, a LNP includes both DSPC and DOPE.

In one embodiment, a non-cationic helper lipid for use in an LNP is selected from the group consisting of: DSPC, DMPE, and DOPC or combinations thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

Examples of phospholipids include, but are not limited to, the following:

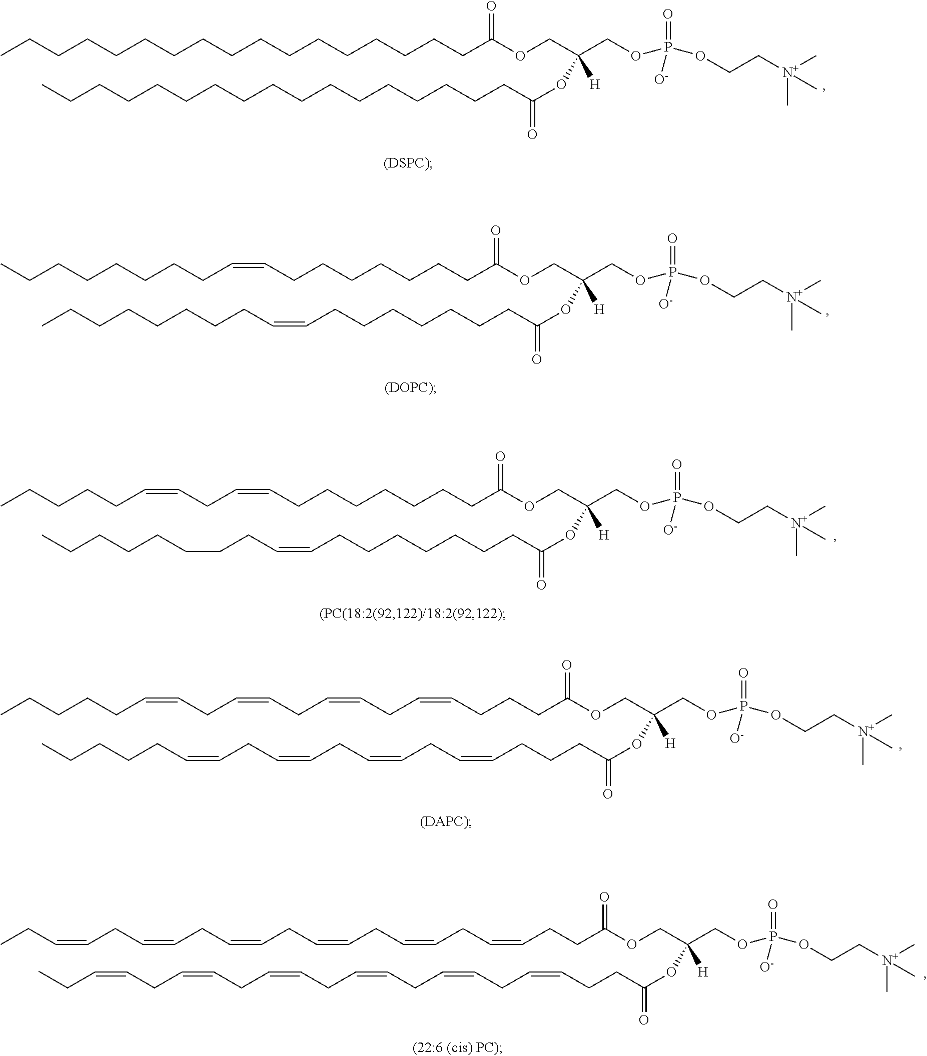

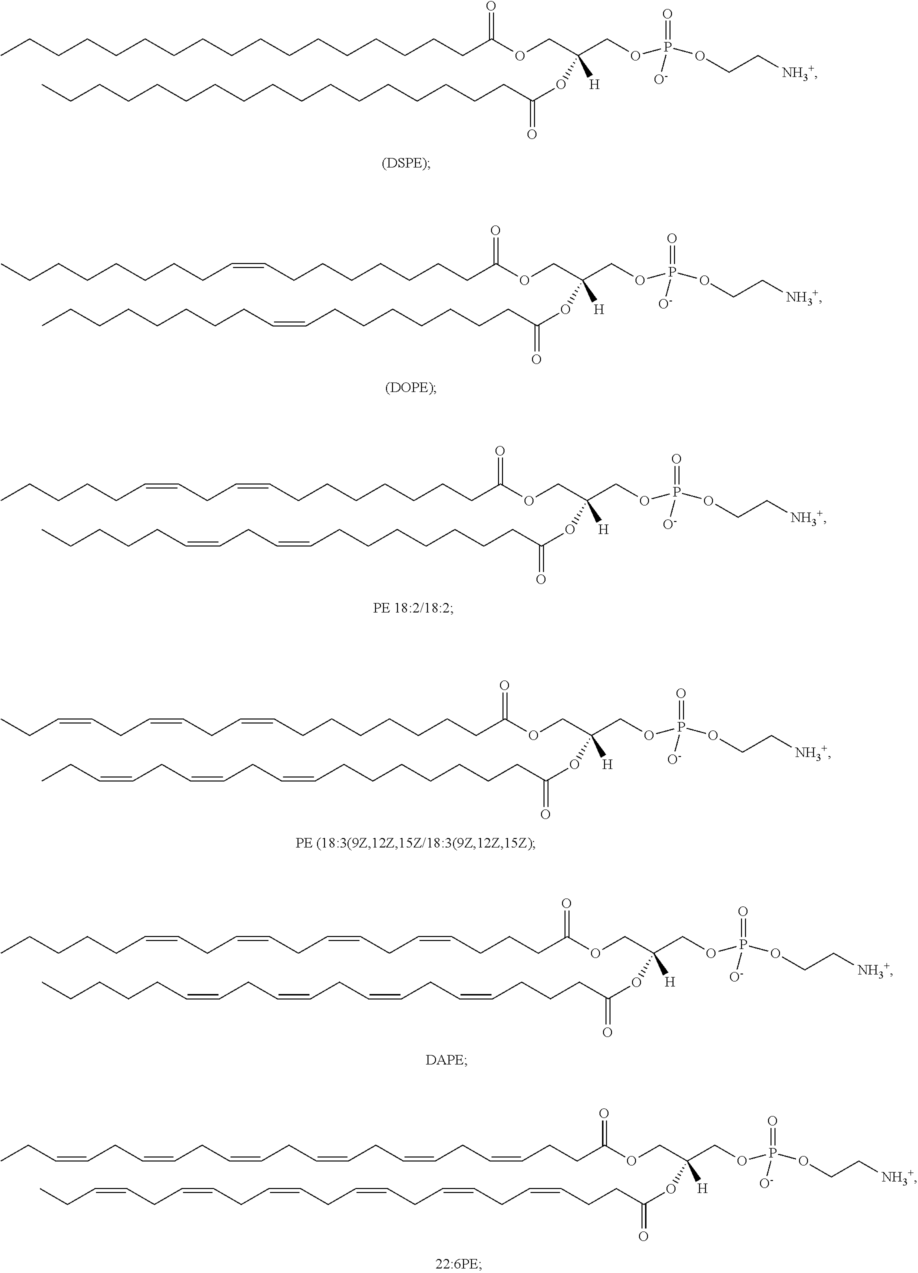

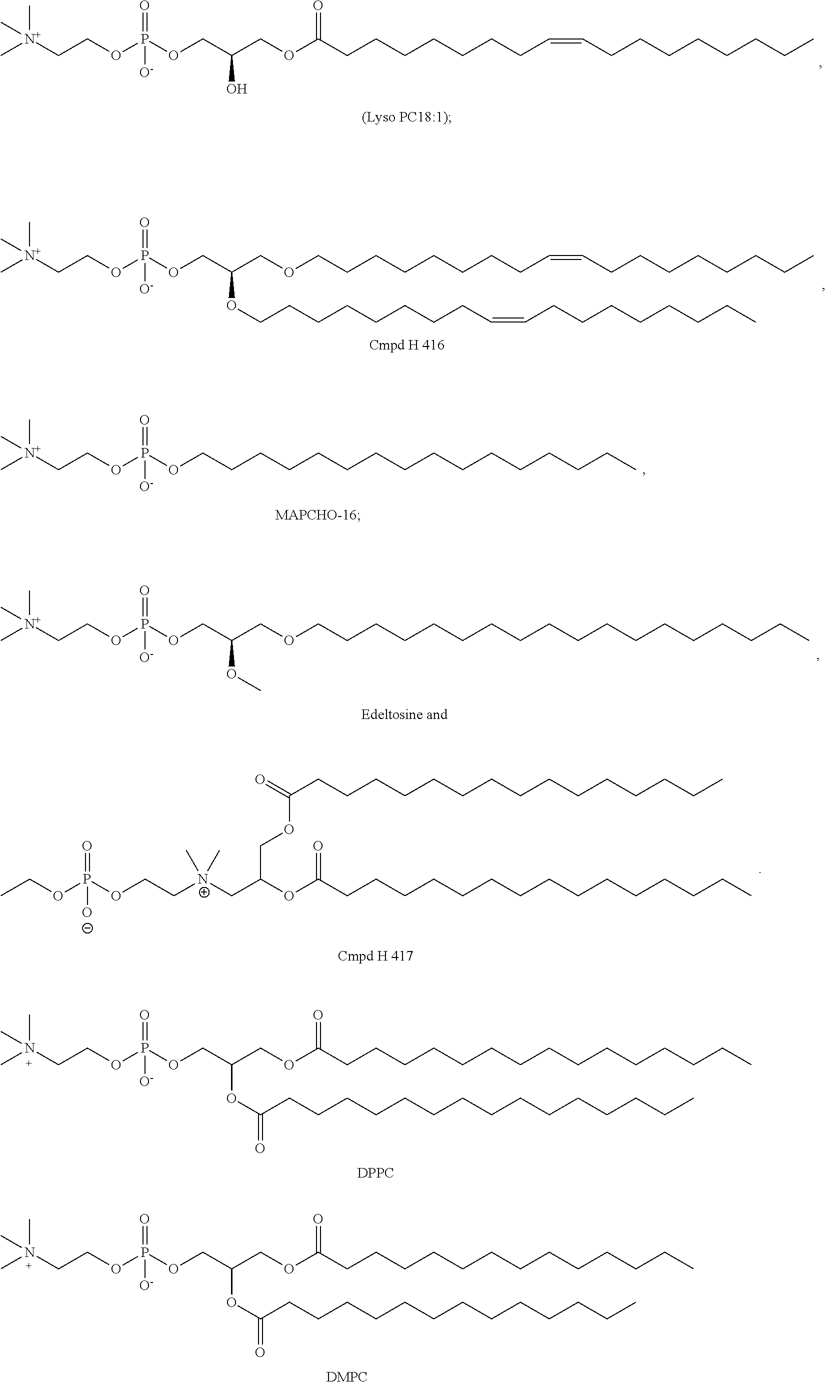

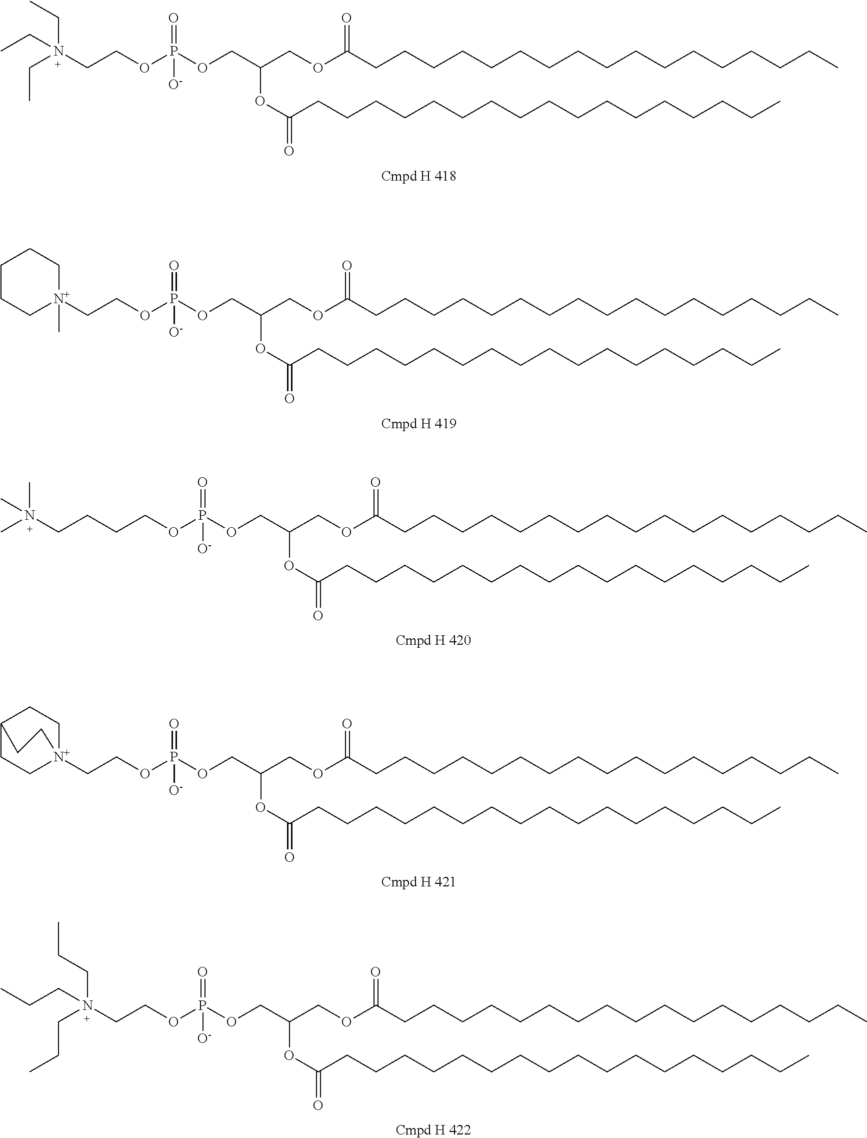

In certain embodiments, a phospholipid useful or potentially useful in the present invention is an analog or variant of DSPC (1,2-dioctadecanoyl-sn-glycero-3-phosphocholine). In certain embodiments, a phospholipid useful or potentially useful in the present invention is a compound of Formula (H IX):

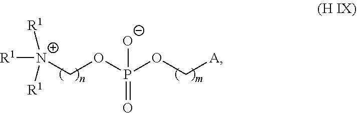

or a salt thereof, wherein:

each R¹ is independently optionally substituted alkyl; or optionally two R′ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

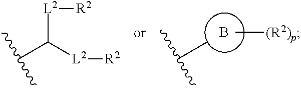

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;

each instance of R² is independently optionally substituted C₁-30 alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

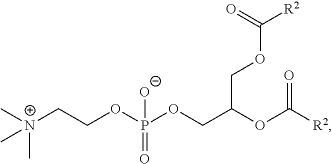

wherein each instance of R² is independently unsubstituted alkyl, unsubstituted alkenyl, or unsubstituted alkynyl.

i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

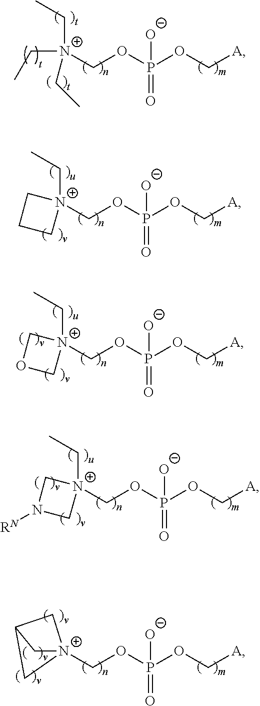

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, the compound of Formula (H IX) is of one of the following formulae:

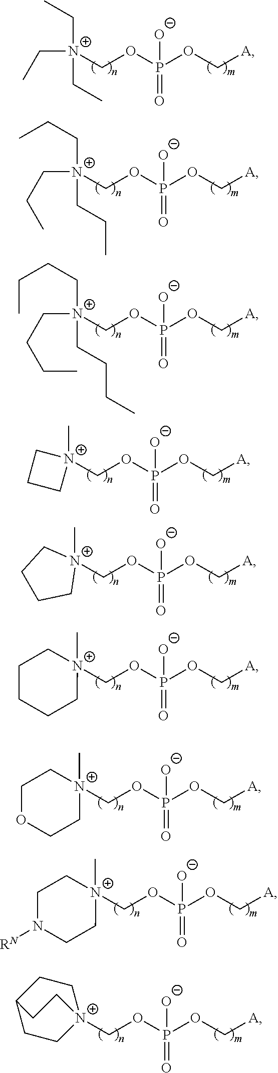

or a salt thereof.

In certain embodiments, a compound of Formula (H IX) is one of the following:

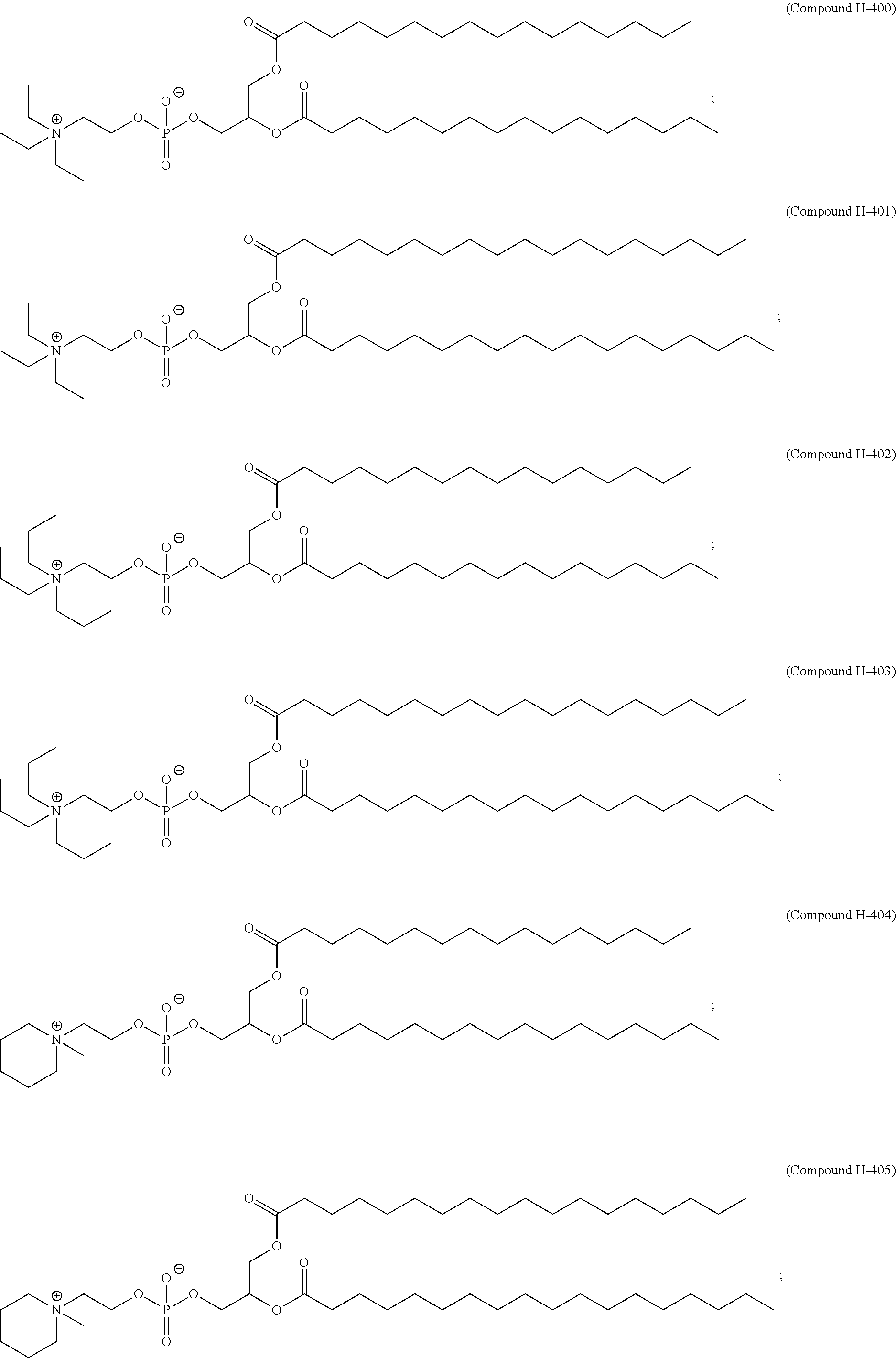

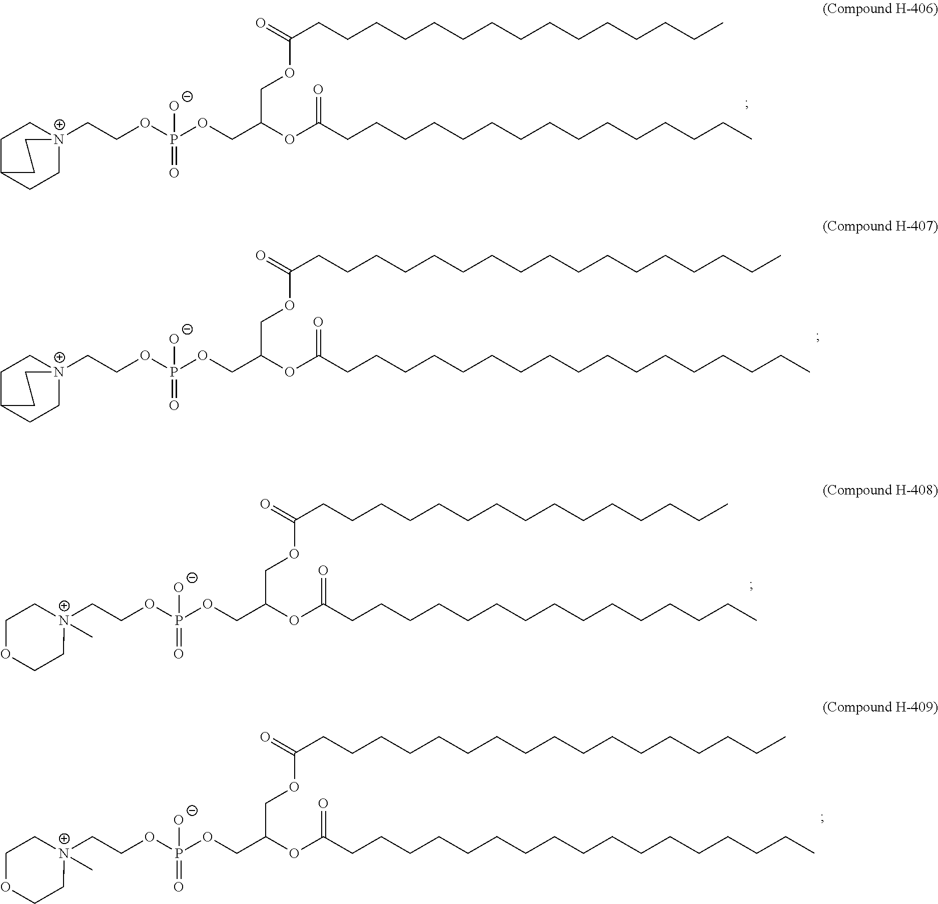 [SMILES: CCCCCCCCCCCCCCCC(=O)OCC(COP(=O)([O-])OCC[N+]12CCC(CC1)CC2)OC(=O)CCCCCCCCCCCCCCC ; CCCCCCCCCCCCCCCCCC(=O)OCC(COP(=O)([O-])OCC[N+]12CCC(CC1)CC2)OC(=O)CCCCCCCCCCCCCCCCC ; CCCCCCCCCCCCCCCC(=O)OCC(COP(=O)([O-])OCC[N+]1(C)CCOCC1)OC(=O)CCCCCCCCCCCCCCC ; CCCCCCCCCCCCCCCCCC(=O)OCC(COP(=O)([O-])OCC[N+]1(C)CCOCC1)OC(=O)CCCCCCCCCCCCCCCCC]

or a salt thereof.

In one embodiment, an LNP comprises Compound H-409 as a non-cationic helper lipid.

(ii) Phospholipid Tail Modifications

In certain embodiments, a phospholipid useful or potentially useful in the present invention comprises a modified tail. In certain embodiments, a phospholipid useful or potentially useful in the present invention is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof.

For example, in certain embodiments, the compound of (H IX) is of Formula (H IX-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H IX) is of Formula (H IX-c):

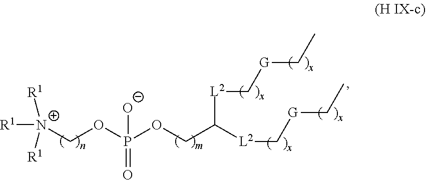

or a salt thereof, wherein:
each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H IX-c) is of Formula (H IX-c-1):

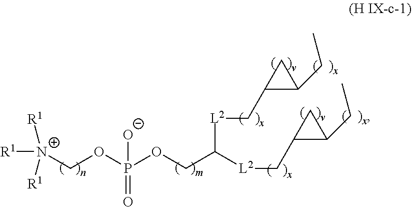

or salt thereof, wherein:
each instance of v is independently 1, 2, or 3.

In certain embodiments, the compound of Formula (H IX-c) is of Formula (H IX-c-2):

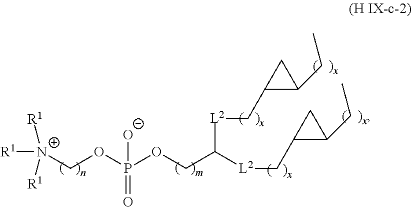

or a salt thereof.

In certain embodiments, the compound of Formula (IX-c) is of the following formula:

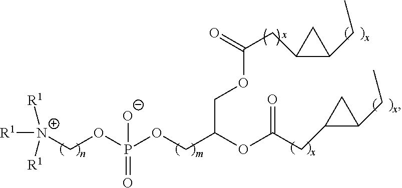

or a salt thereof.

In certain embodiments, the compound of Formula (H IX-c) is the following:

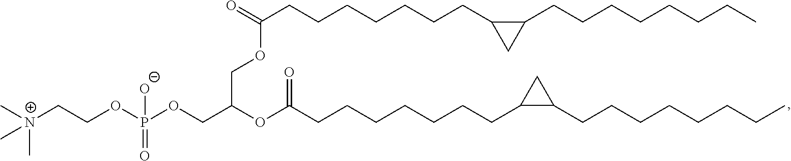 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H IX-c) is of Formula (H IX-c-3):

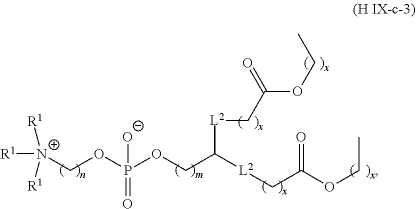

or a salt thereof.

In certain embodiments, the compound of Formula (H IX-c) is of the following formulae:

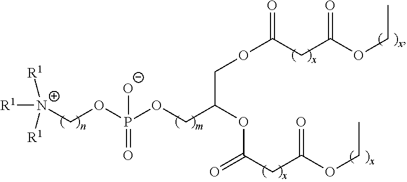

or a salt thereof.

In certain embodiments, the compound of Formula (H IX-c) is the following:

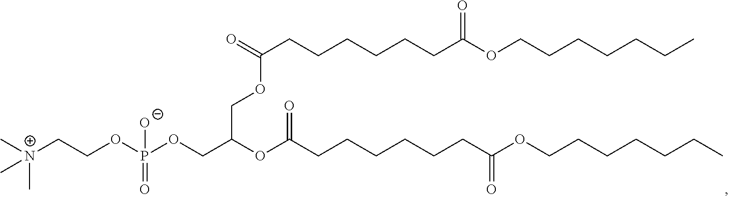 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (H IX), wherein n is 1, 3, 4, 5, 6, 7, 8, 9, or 10. For example, in certain embodiments, a compound of Formula (H IX) is of one of the following formulae:

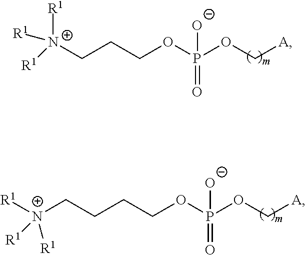

or a salt thereof.

In certain embodiments, a compound of Formula (H IX) is one of the following:

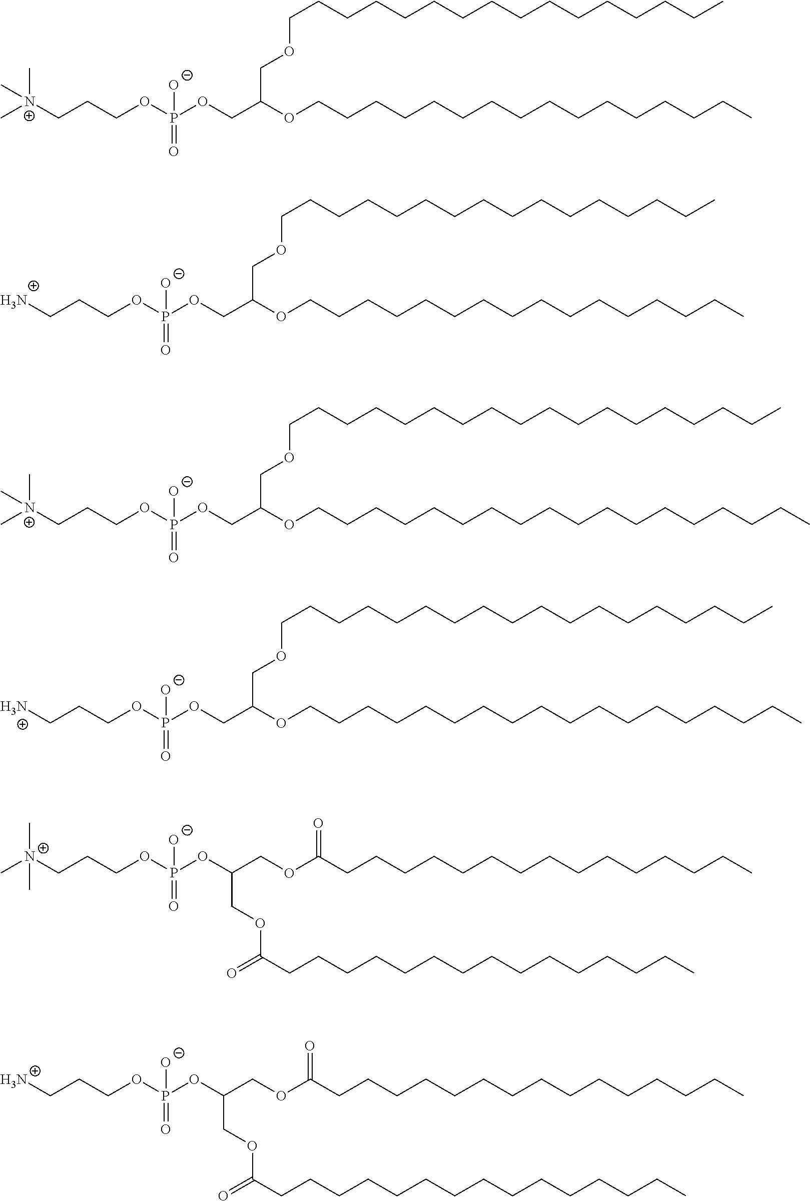

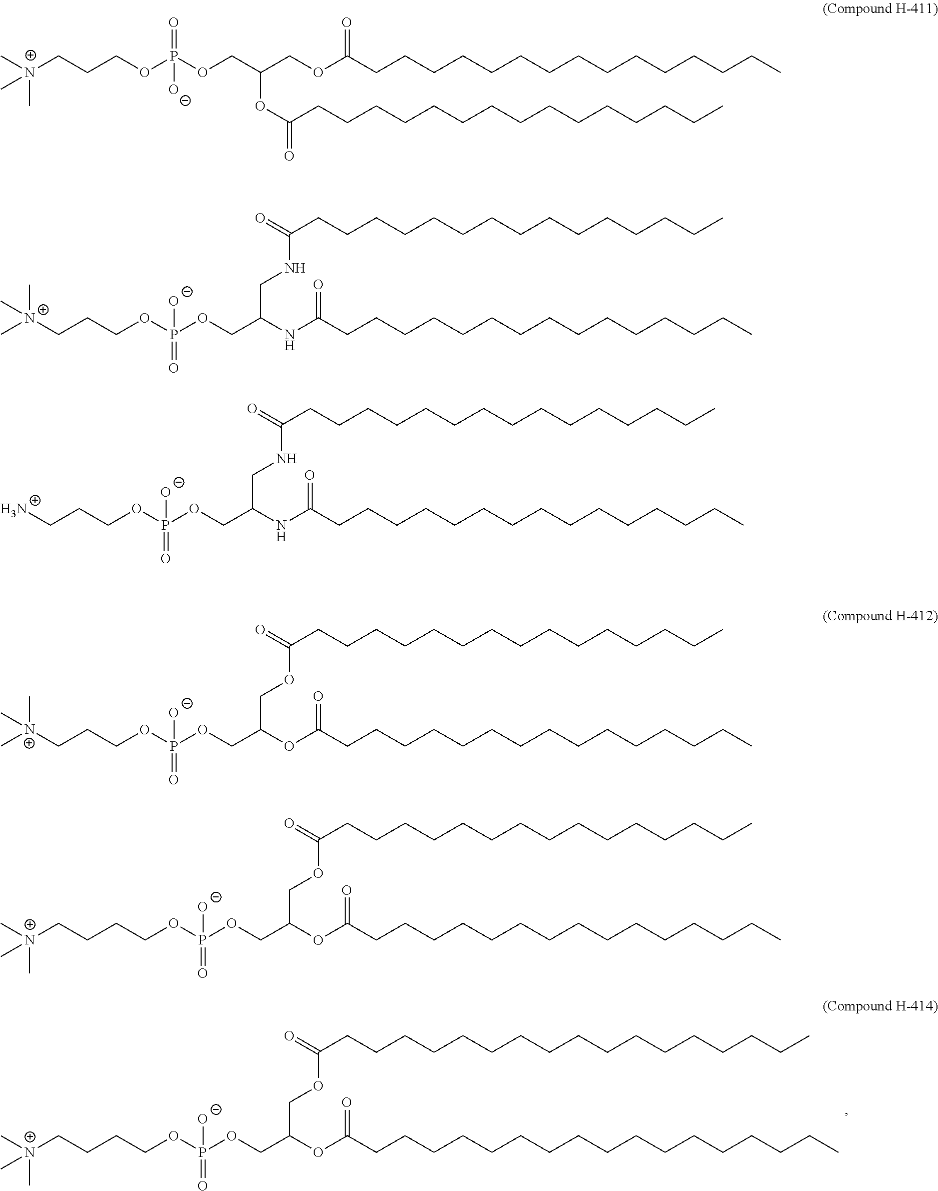

or salts thereof.

In certain embodiments, an alternative lipid is used in place of a phospholipid of the invention. Non-limiting examples of such alternative lipids include the following:

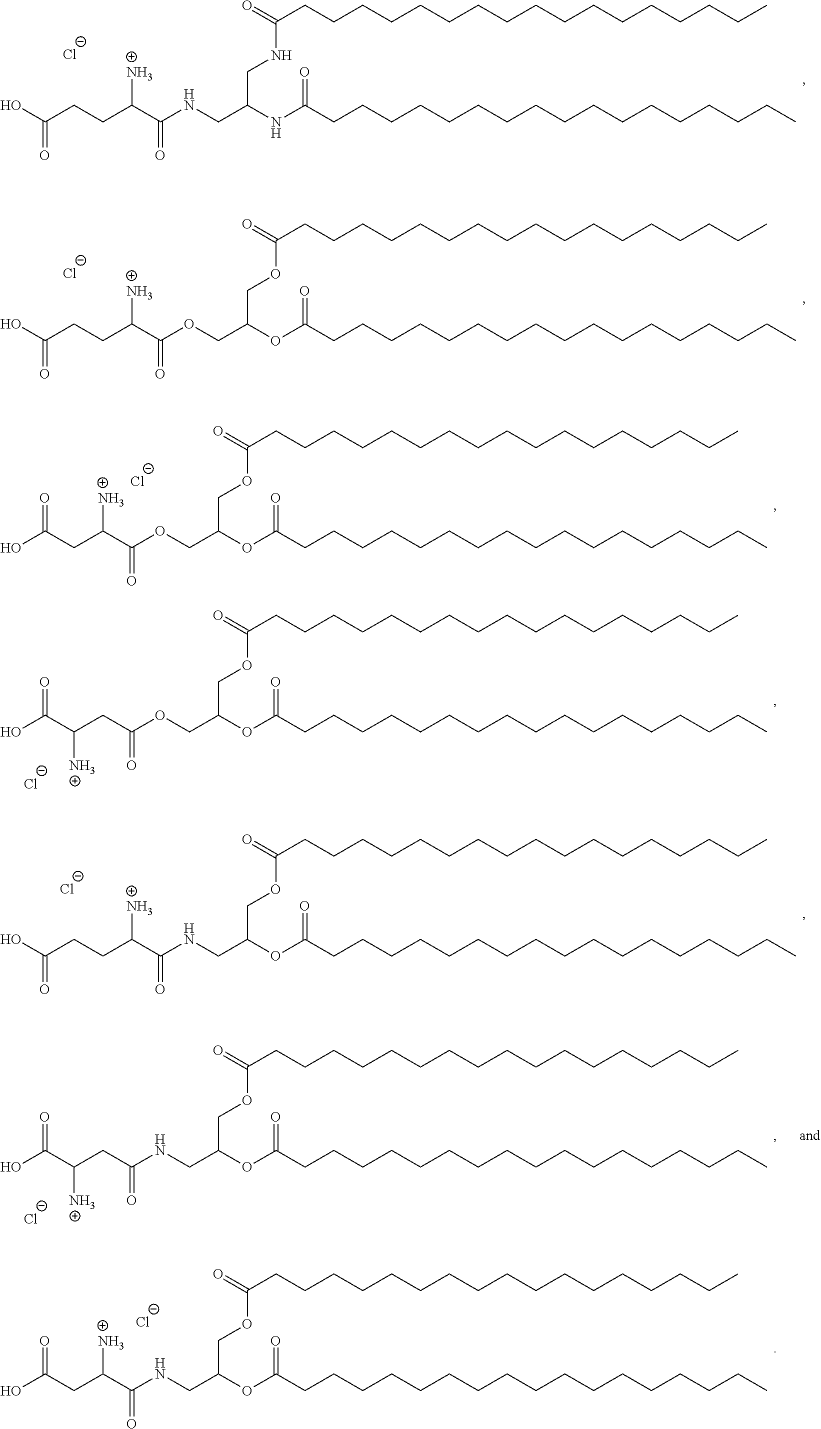

Phospholipid Tail Modifications

In certain embodiments, a phospholipid useful in the present invention comprises a modified tail. In certain embodiments, a phospholipid useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H I) is of Formula (H I-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N}), —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H I-a) is of Formula (H I-c):

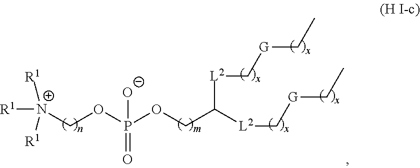

or a salt thereof, wherein:

each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N}), —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H I-c) is of Formula (H I-c-1):

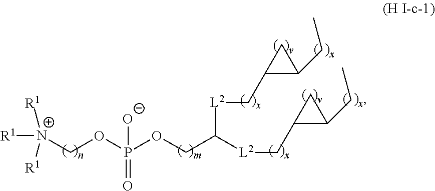

or salt thereof, wherein:

each instance of v is independently 1, 2, or 3.

In certain embodiments, the compound of Formula (H I-c) is of Formula (H I-c-2):

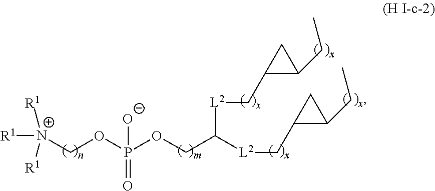

or a salt thereof.

In certain embodiments, the compound of Formula (I-c) is of the following formula:

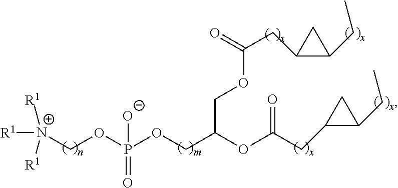

or a salt thereof.

In certain embodiments, the compound of Formula (H I-c) is the following:

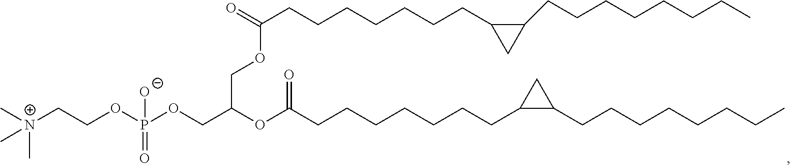 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H I-c) is of Formula (H I-c-3):

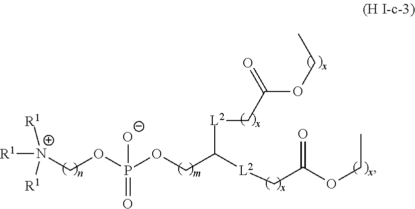

or a salt thereof.

In certain embodiments, the compound of Formula (H I-c) is of the following formulae:

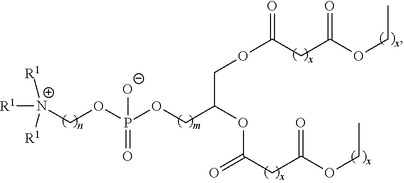

or a salt thereof.

In certain embodiments, the compound of Formula (H I-c) is the following:

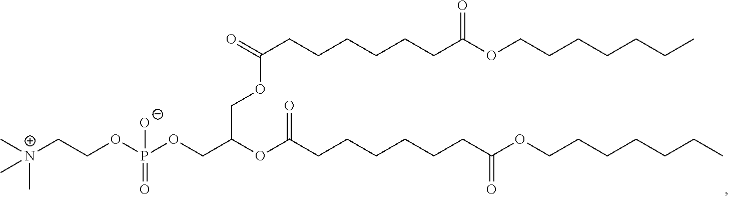 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

Phosphocholine Linker Modifications

In certain embodiments, a phospholipid useful in the present invention comprises a modified phosphocholine moiety, wherein the alkyl chain linking the quaternary amine to the phosphoryl group is not ethylene (e.g., n is not 2). Therefore, in certain embodiments, a phospholipid useful in the present invention is a compound of Formula (H I), wherein n is 1, 3, 4, 5, 6, 7, 8, 9, or 10. For example, in certain embodiments, a compound of Formula (H I) is of one of the following formulae:

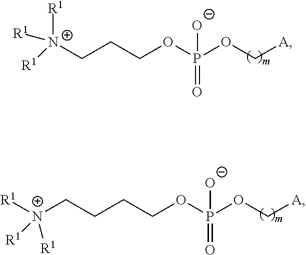

or a salt thereof.

In certain embodiments, a compound of Formula (H I) is one of the following:

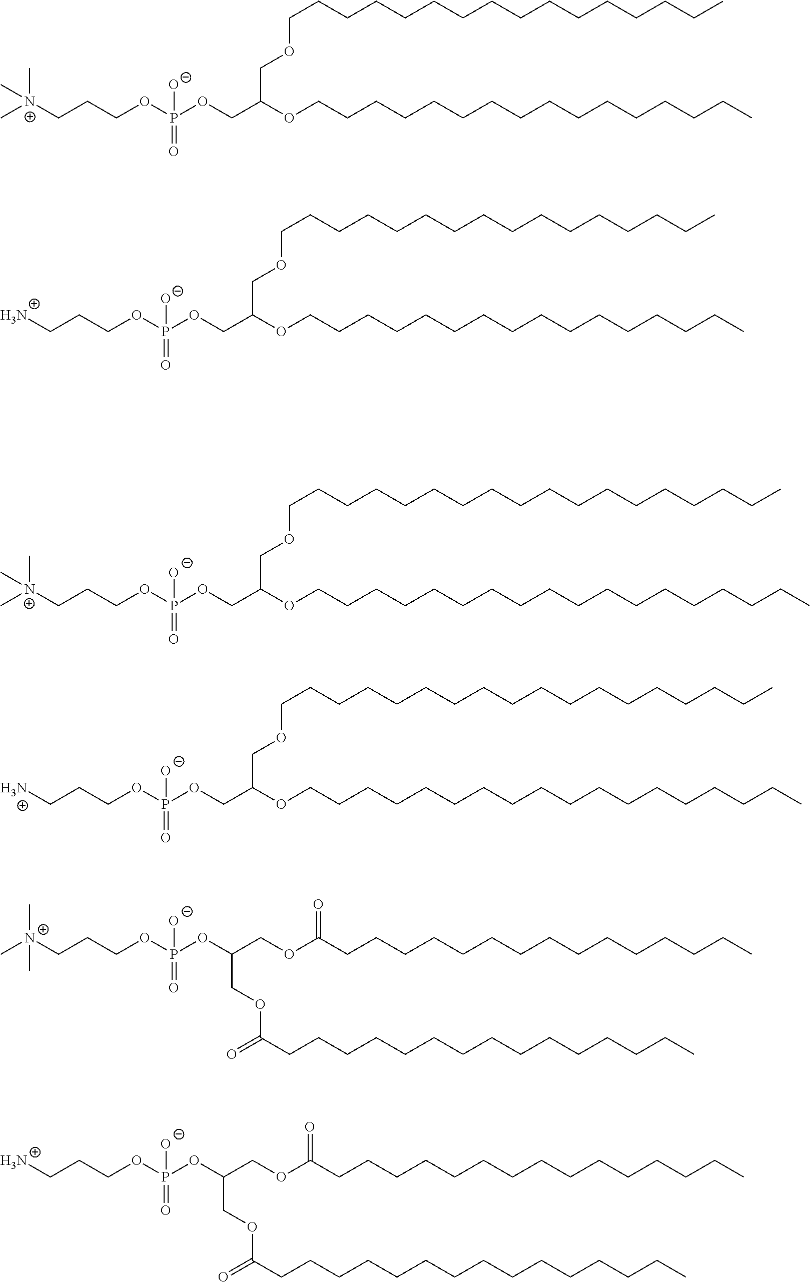

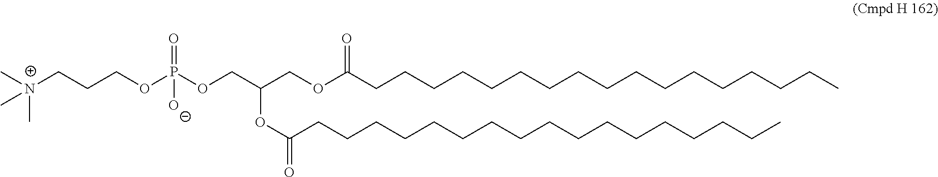 [SMILES: CCCCCCCCCCCCCCCCCC(=O)OCC(COP(=O)([O-])OCCC[N+](C)(C)C)OC(=O)CCCCCCCCCCCCCCCCC]

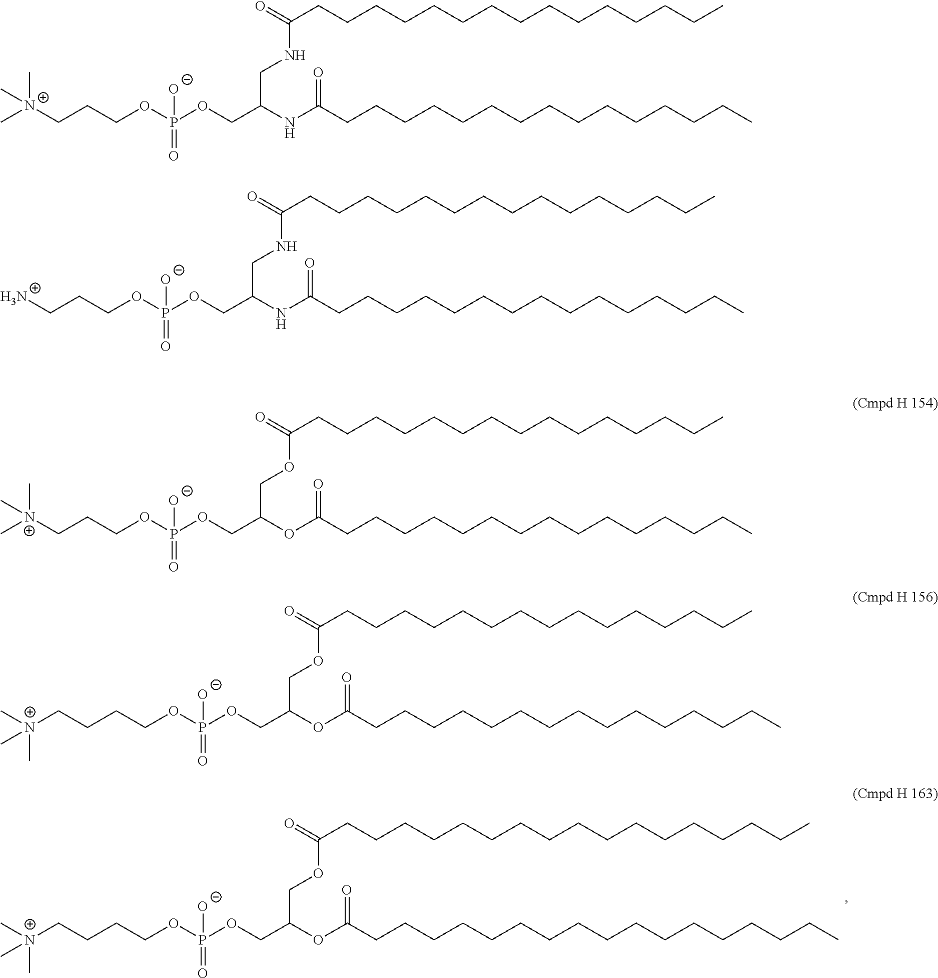

or salts thereof.

Numerous LNP formulations having phospholipids other than DSPC were prepared and tested for activity, as demonstrated in the examples below.

### Phospholipid Substitute or Replacement

In some embodiments, the lipid-based composition (e.g., lipid nanoparticle) comprises an oleic acid or an oleic acid analog in place of a phospholipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the lipid-based composition (e.g., lipid nanoparticle) comprises a different zwitterionic group in place of a phospholipid.

Exemplary phospholipid substitutes and/or replacements are provided in Published PCT Application WO 2017/099823, herein incorporated by reference.

Exemplary phospholipid substitutes and/or replacements are provided in Published PCT Application WO 2017/099823, herein incorporated by reference.

(iv) PEG Lipids

Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

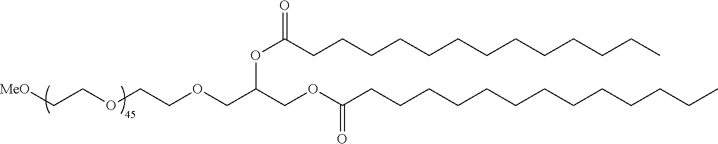

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In some embodiments, the PEG lipid is a compound of Formula (PI):

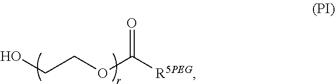

or a salt or isomer thereof, wherein:

r is an integer between 1 and 100;

R^{5PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

For example, R^{5PEG} is C₁₇ alkyl. For example, the PEG lipid is a compound of Formula (PI-a):

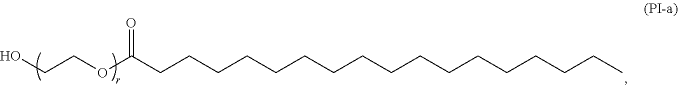

or a salt or isomer thereof, wherein r is an integer between 1 and 100.

For example, the PEG lipid is a compound of the following formula:

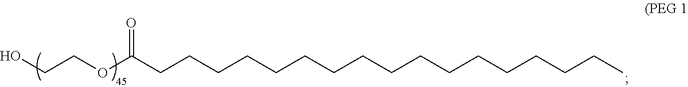

also referred to as Compound 428 or Compound I below),
or a salt or isomer thereof.

The PEG lipid may be a compound of Formula (PII):

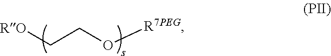

or a salt or isomer thereof, wherein:

s is an integer between 1 and 100;

R″ is a hydrogen, C₁₋₁₀ alkyl, or an oxygen protecting group;

R^{7PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

In some embodiments, R⁷PEG is C₁₀₋₆₀ alkyl, and one or more of the methylene groups of R^{7PEG} are replaced with —C(O)—. For example, R^{7PEG} is C₃₁ alkyl, and two of the methylene groups of R^{7PEG} are replaced with —C(O)—.

In some embodiments, R″ is methyl.

In some embodiments, the PEG lipid is a compound of Formula (PII-a):

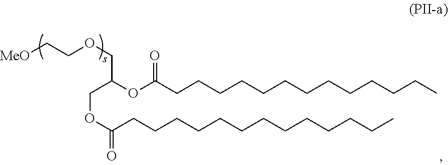

or a salt or isomer thereof, wherein s is an integer between 1 and 100.

For example, the PEG lipid is a compound of the following formula:

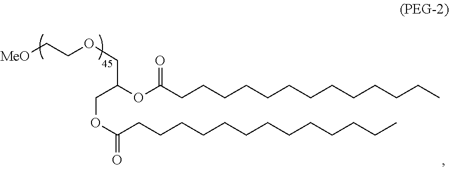

or a salt or isomer thereof.

In certain embodiments, a PEG lipid useful in the present invention is a compound of

Formula (PIII). Provided herein are compounds of Formula (PIII):

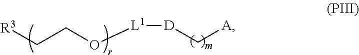

or salts thereof, wherein:

R³ is —OR^{O};

R^{O} is hydrogen, optionally substituted alkyl, or an oxygen protecting group; r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, —OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

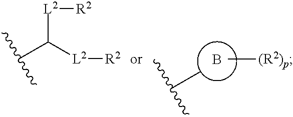

each instance of L² is independently a bond or optionally substituted C₁₋₆ alkylene, wherein one methylene unit of the optionally substituted C₁₋₆ alkylene is optionally replaced with 0, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂₀, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (PIII) is a PEG-OH lipid (i.e., R³ is —OR^{O}, and R^{O} is hydrogen). In certain embodiments, the compound of Formula (PIII) is of Formula (PIII-OH):

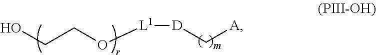

or a salt thereof.

In certain embodiments, D is a moiety obtained by click chemistry (e.g., triazole). In certain embodiments, the compound of Formula (PIII) is of Formula (PIII-a-1) or (PIII-a-2):

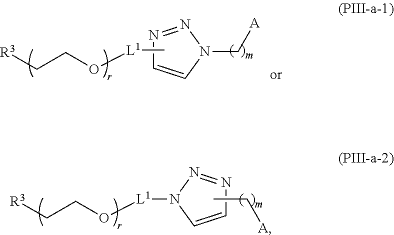

or a salt thereof.

In certain embodiments, the compound of Formula (PIII) is of one of the following formulae:

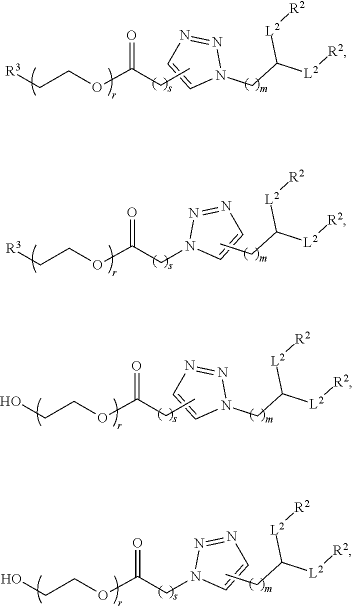

or a salt thereof, wherein

s is 0, 1, 2, 3, 4, 5, 6, 7, 8, 9, or 10.

In certain embodiments, the compound of Formula (PIII) is of one of the following formulae:

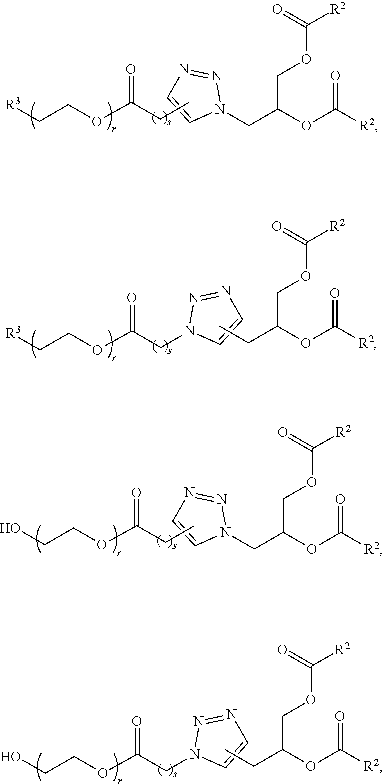

or a salt thereof.

In certain embodiments, a compound of Formula (PIII) is of one of the following formulae:

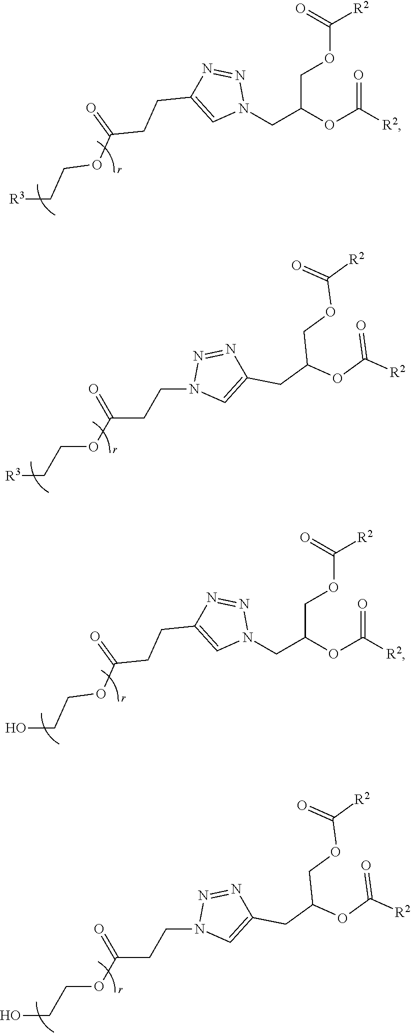

or a salt thereof.

In certain embodiments, a compound of Formula (PIII) is of one of the following formulae:

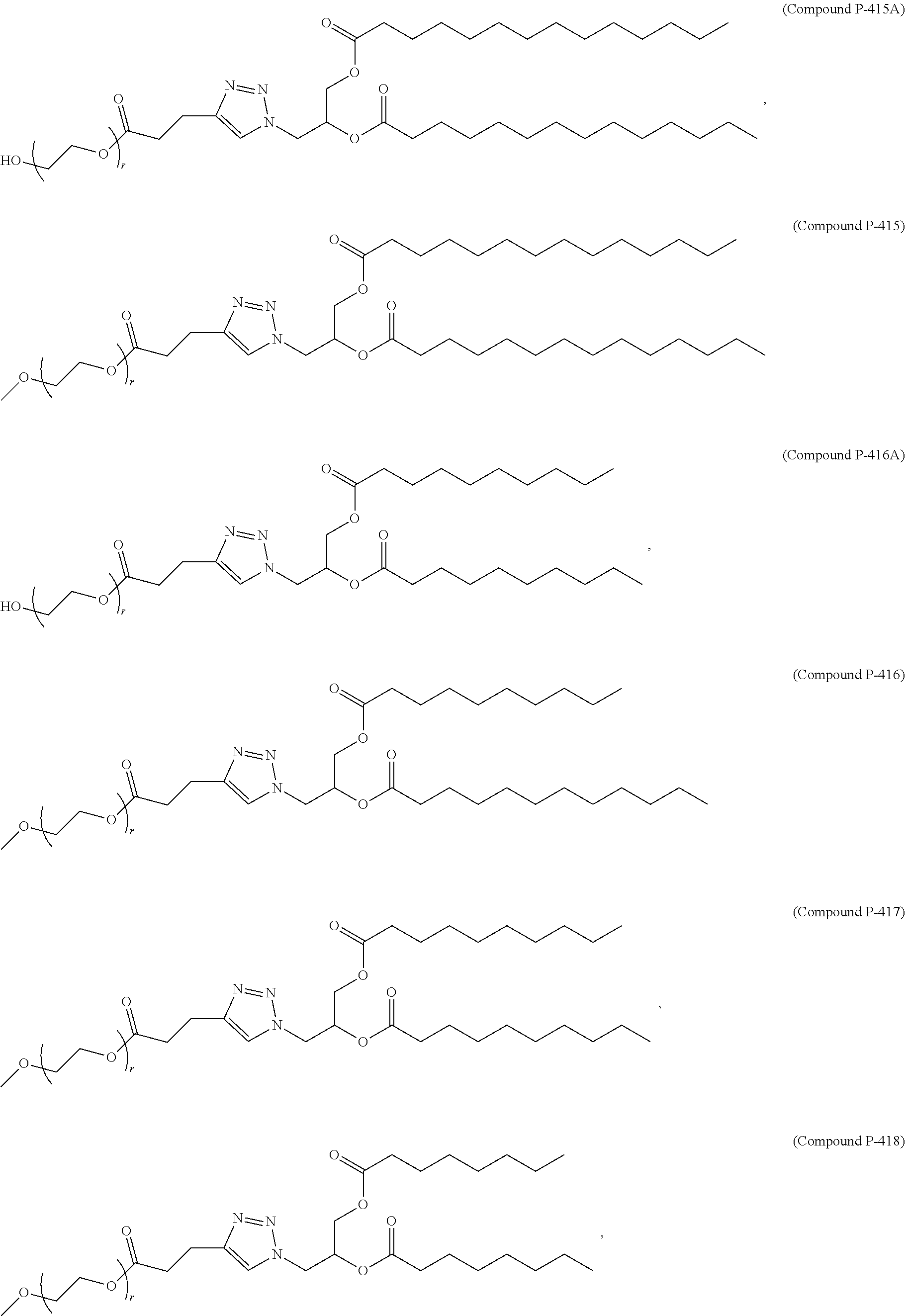

or a salt thereof.

In certain embodiments, D is a moiety cleavable under physiological conditions (e.g., ester, amide, carbonate, carbamate, urea). In certain embodiments, a compound of Formula (PIII) is of Formula (PIII-b-1) or (PIII-b-2):

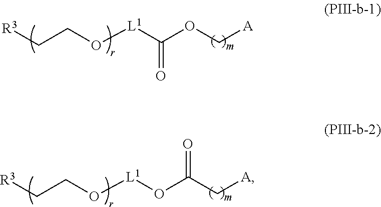

or a salt thereof.

In certain embodiments, a compound of Formula (PIII) is of Formula (PIII-b-1-OH) or (PIII-b-2-OH):

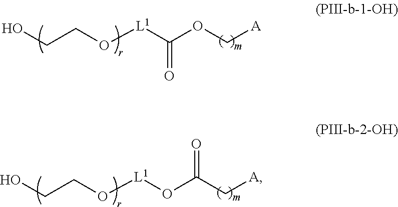

or a salt thereof.

In certain embodiments, the compound of Formula (PIII) is of one of the following formulae:

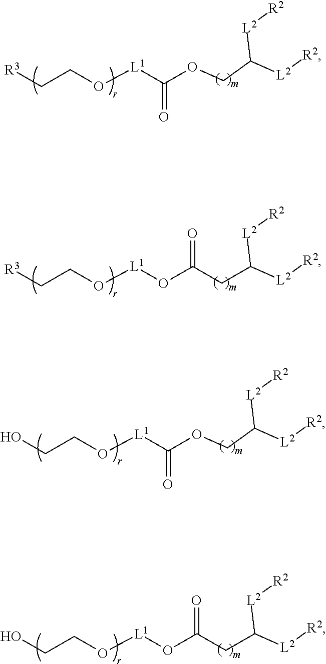

or a salt thereof.

In certain embodiments, a compound of Formula (PIII) is of one of the following formulae:

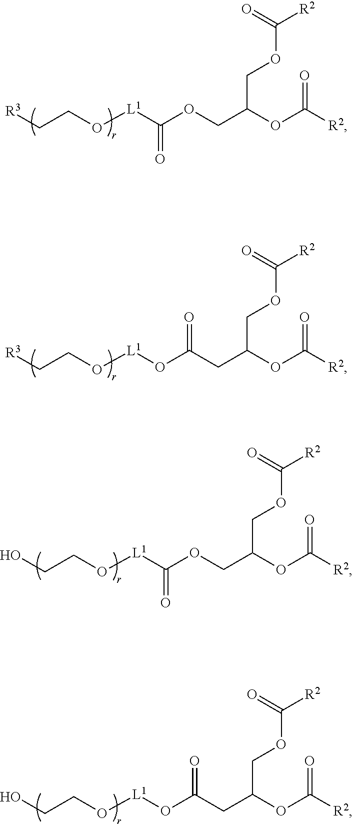

or a salt thereof.

In certain embodiments, a compound of Formula (PIII) is of one of the following formulae:

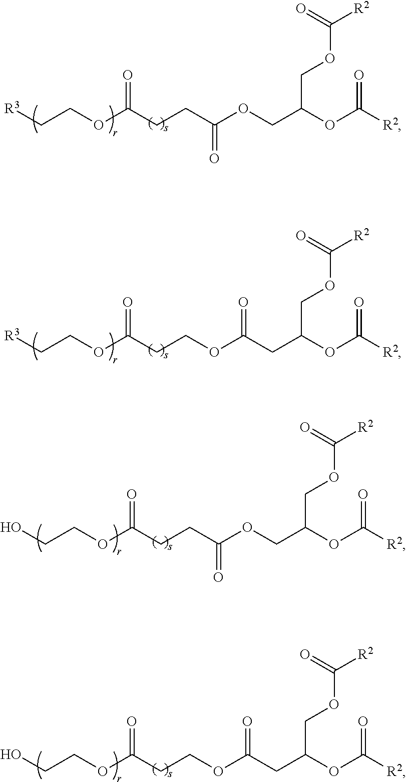

or a salt thereof.

In certain embodiments, a compound of Formula (PIII) is of one of the following formulae:

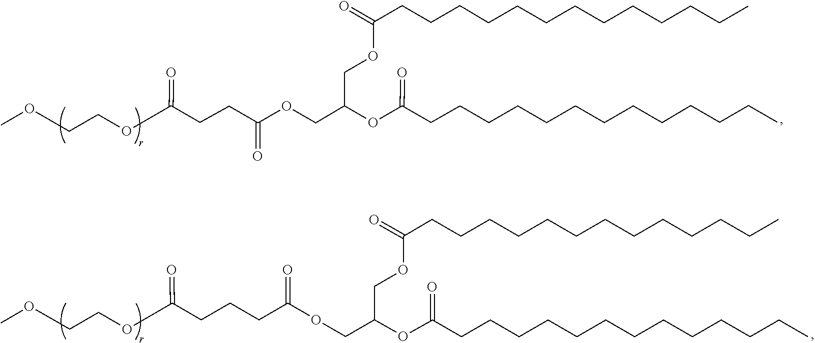

or salts thereof.

In certain embodiments, a PEG lipid useful in the present invention is a PEGylated fatty acid. In certain embodiments, a PEG lipid useful in the present invention is a compound of Formula (PIV). Provided herein are compounds of Formula (PIV):

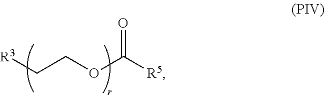

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), —NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)₀, OS(O)₂, —S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (PIV is of Formula (PIV-OH):

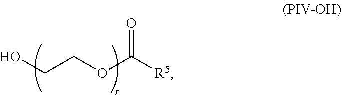

or a salt thereof. In some embodiments, r is 40-50. In some embodiments, r is 45.

In certain embodiments, a compound of Formula (PIV) is of one of the following formulae:

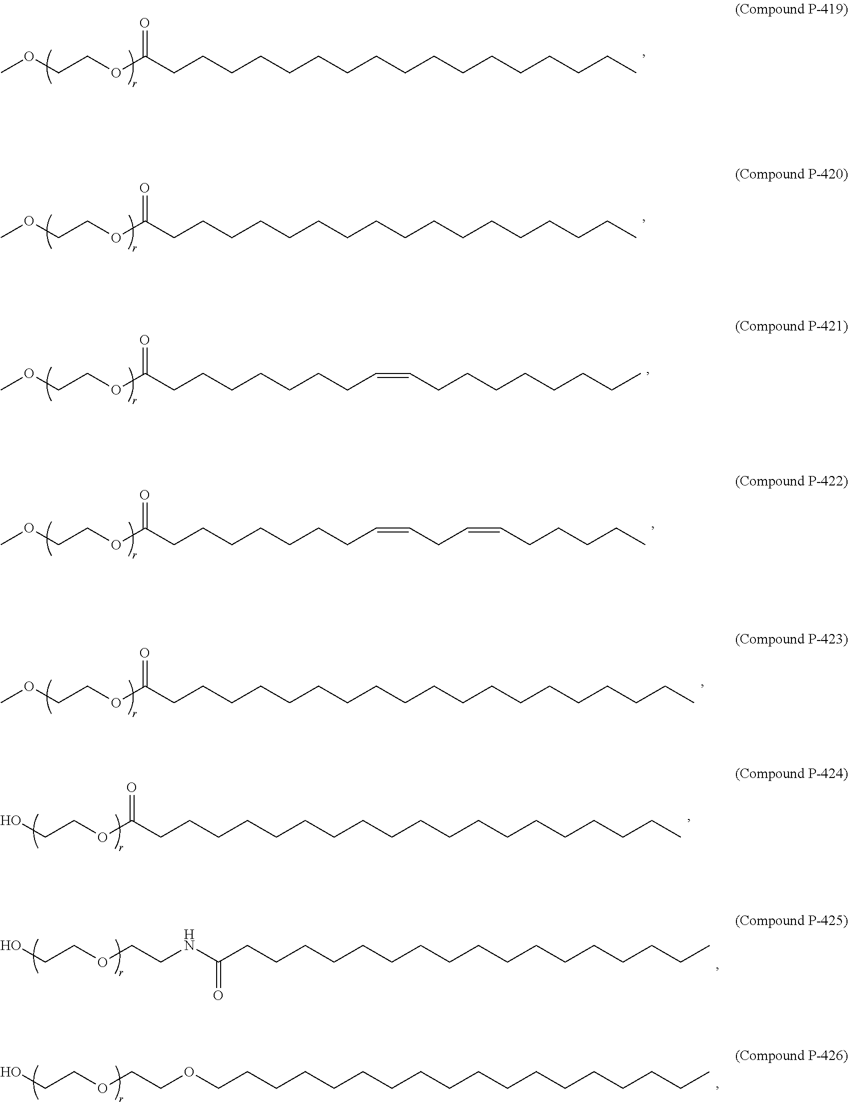

or a salt thereof. In some embodiments, r is 40-50. In some embodiments, r is 45.
In yet other embodiments the compound of Formula (PIV) is:

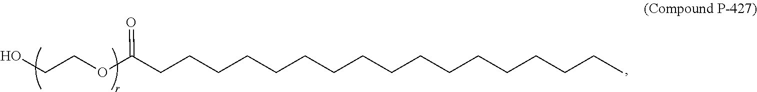

or a salt thereof.
In one embodiment, the compound of Formula (PIV) is

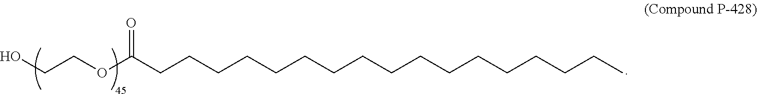

In one aspect, provided herein are lipid nanoparticles (LNPs) comprising PEG lipids of Formula (PV):

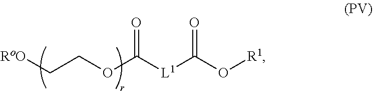

or pharmaceutically acceptable salts thereof; wherein:

L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;

R¹ is optionally substituted C₅₋₃₀ alkyl, optionally substituted C₅₋₃₀ alkenyl, or optionally substituted C₅₋₃₀ alkynyl;

R^{O} is hydrogen, optionally substituted alkyl, optionally substituted acyl, or an oxygen protecting group; and

r is an integer from 2 to 100, inclusive.

In certain embodiments, the PEG lipid of Formula (PV) is of the following formula:

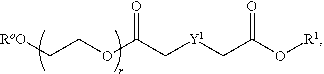

or a pharmaceutically acceptable salt thereof; wherein:

Y¹ is a bond, —CR₂—, —O—, —NR^{N}—, or —S—;

each instance of R is independently hydrogen, halogen, or optionally substituted alkyl; and

R^{N} is hydrogen, optionally substituted alkyl, optionally substituted acyl, or a nitrogen protecting group.

In certain embodiments, the PEG lipid of Formula (PV) is of one of the following formulae:

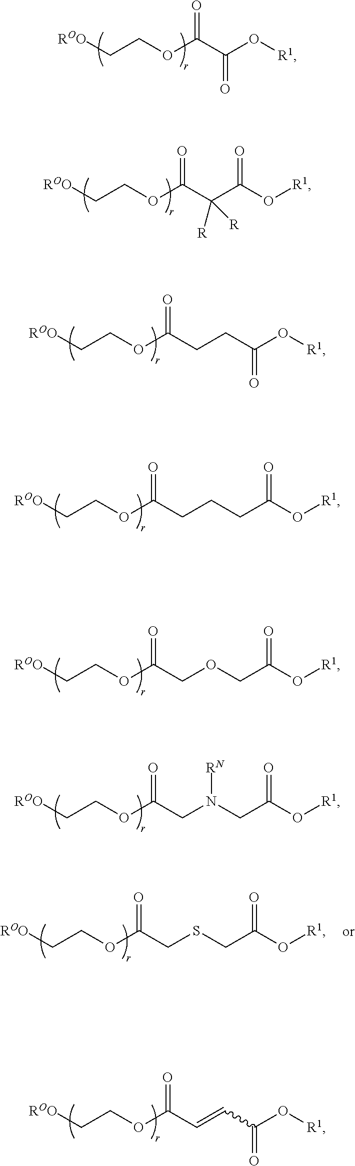

or a pharmaceutically acceptable salt thereof, wherein:

each instance of R is independently hydrogen, halogen, or optionally substituted alkyl.

In certain embodiments, the PEG lipid of Formula (PV) is of one of the following formulae:

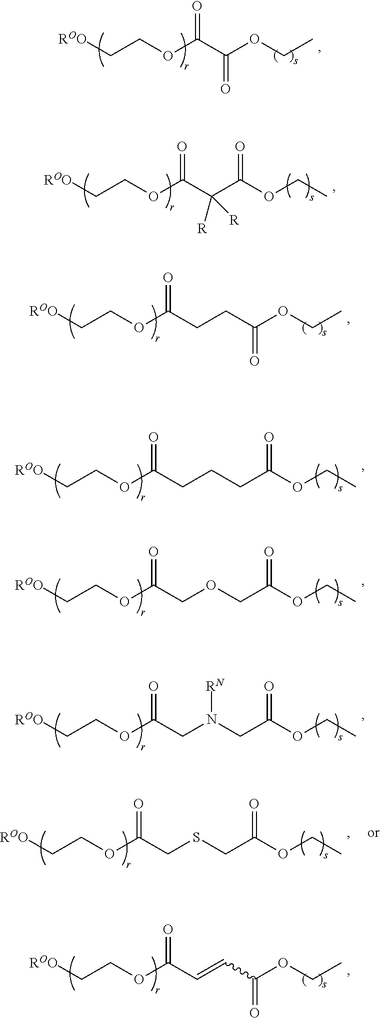

or a pharmaceutically acceptable salt thereof; wherein:

s is an integer from 5-25, inclusive.

In certain embodiments, the PEG lipid of Formula (PV) is of one of the following formulae:

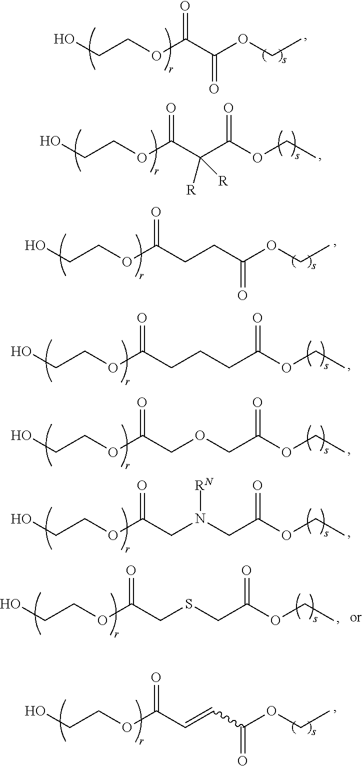

or a pharmaceutically acceptable salt thereof.

In certain embodiments, the PEG lipid of Formula (PV) is selected from the group consisting of:

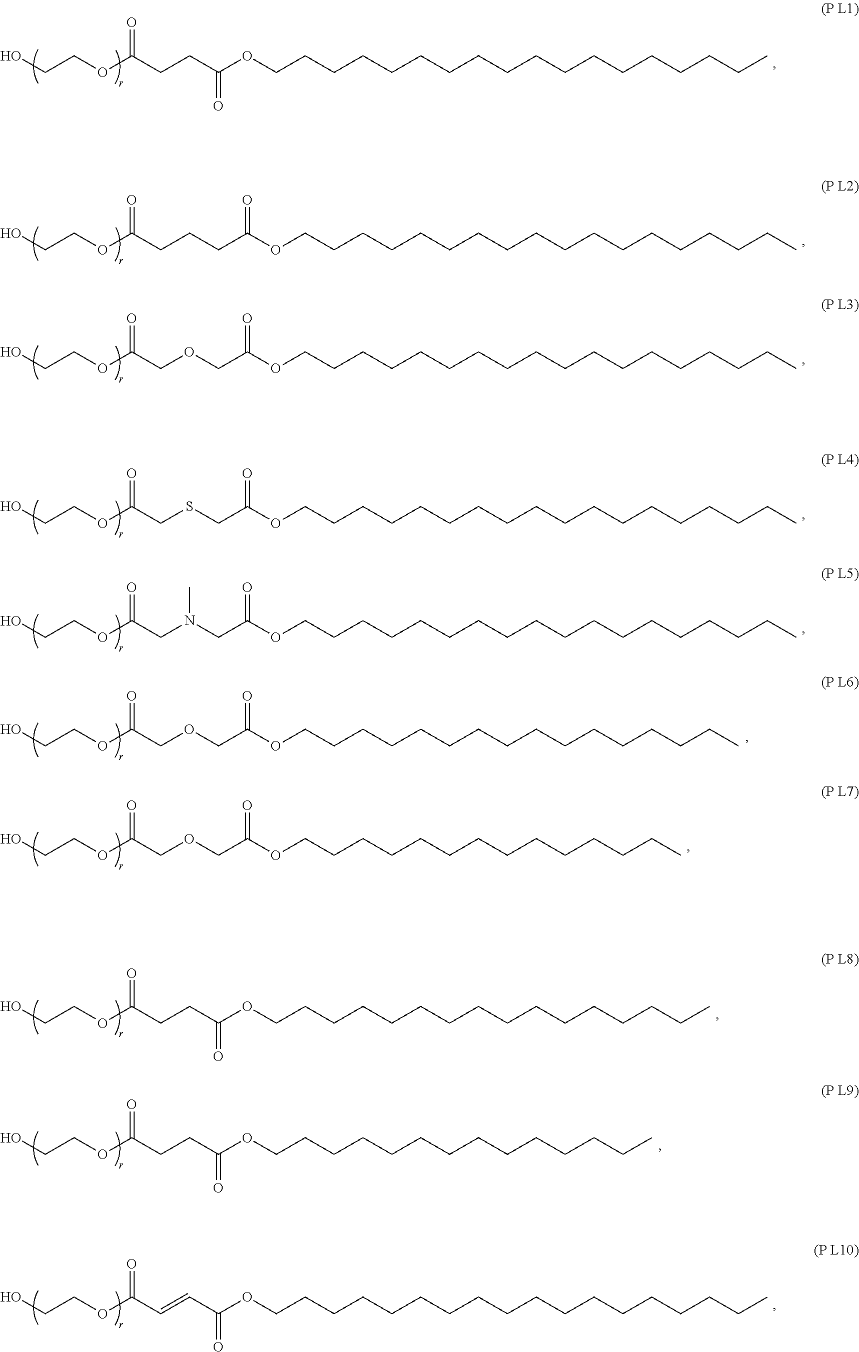

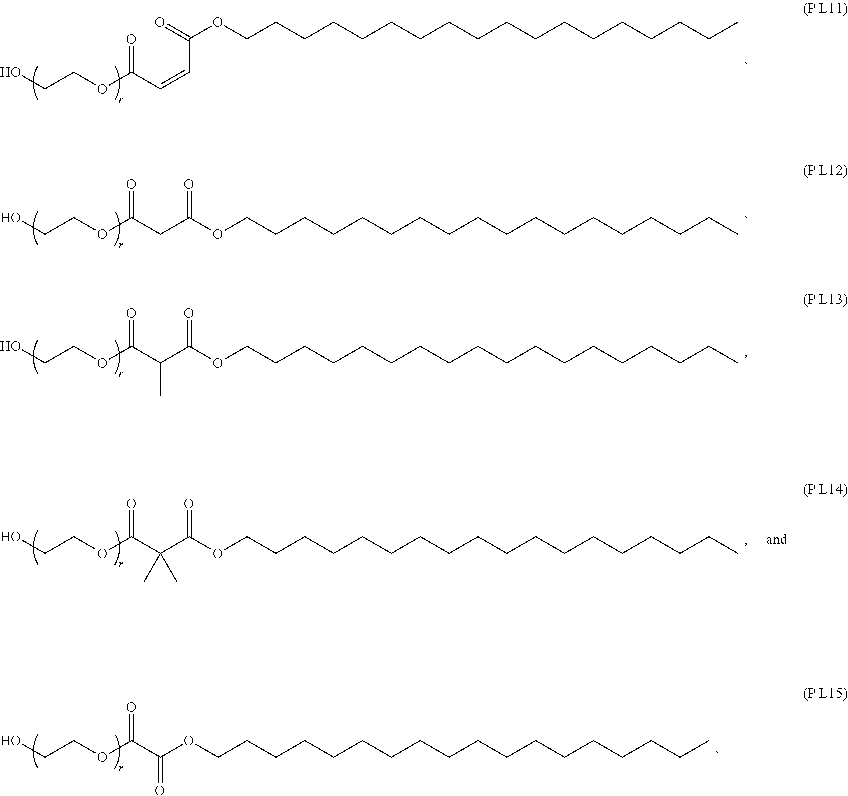

and pharmaceutically acceptable salts thereof.

In another aspect, provided herein are lipid nanoparticles (LNPs) comprising PEG lipids of Formula (PVI):

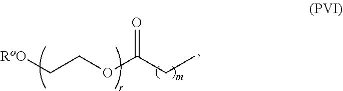

or pharmaceutically acceptable salts thereof; wherein:

R^{O} is hydrogen, optionally substituted alkyl, optionally substituted acyl, or an oxygen protecting group;

r is an integer from 2 to 100, inclusive; and

m is an integer from 5-15, inclusive, or an integer from 19-30, inclusive.

In certain embodiments, the PEG lipid of Formula (PVI) is of one of the following formulae:

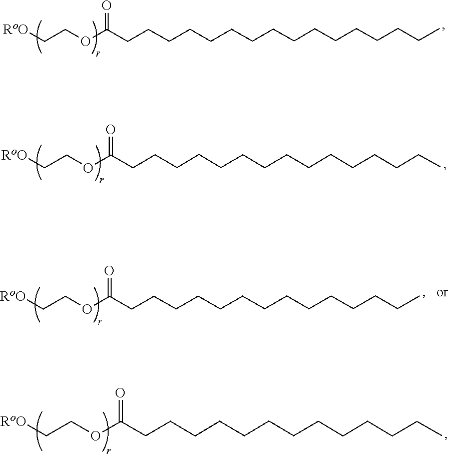

or a pharmaceutically acceptable salt thereof.

In certain embodiments, the PEG lipid of Formula (PVI) is of one of the following formulae:

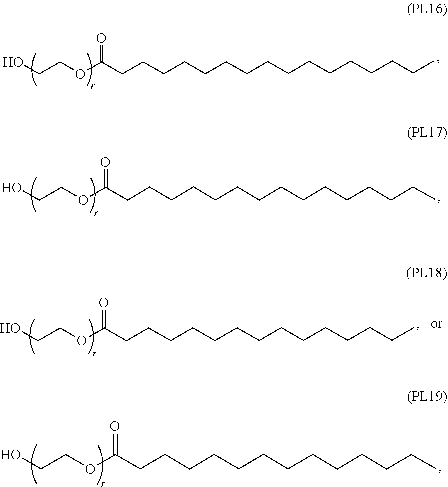

or a pharmaceutically acceptable salt thereof.

In another aspect, provided herein are lipid nanoparticles (LNPs) comprising PEG lipids of Formula (PVII):

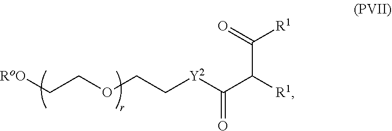

or pharmaceutically acceptable salts thereof, wherein:

Y² is —O—, —NR^{N}—, or —S— each instance of R¹ is independently optionally substituted C₅₋₃₀ alkyl, optionally substituted C₅₋₃₀ alkenyl, or optionally substituted C₅₋₃₀ alkynyl;

R^{O} is hydrogen, optionally substituted alkyl, optionally substituted acyl, or an oxygen protecting group;

R^{N} is hydrogen, optionally substituted alkyl, optionally substituted acyl, or a nitrogen protecting group; and

r is an integer from 2 to 100, inclusive.

In certain embodiments, the PEG lipid of Formula (PVII) is of one of the following formulae:

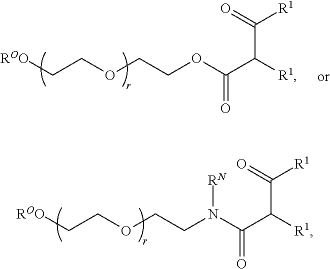

or a pharmaceutically acceptable salt thereof.

In certain embodiments, the PEG lipid of Formula (PVII) is of one of the following formulae:

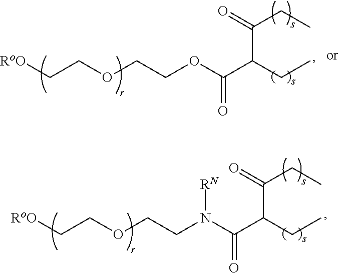

or a pharmaceutically acceptable salt thereof; wherein:

each instance of s is independently an integer from 5-25, inclusive.

In certain embodiments, the PEG lipid of Formula (PVII) is of one of the following formulae:

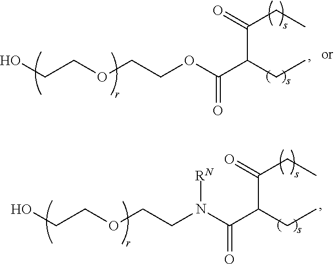

or a pharmaceutically acceptable salt thereof

In certain embodiments, the PEG lipid of Formula (PVII) is selected from the group consisting of:

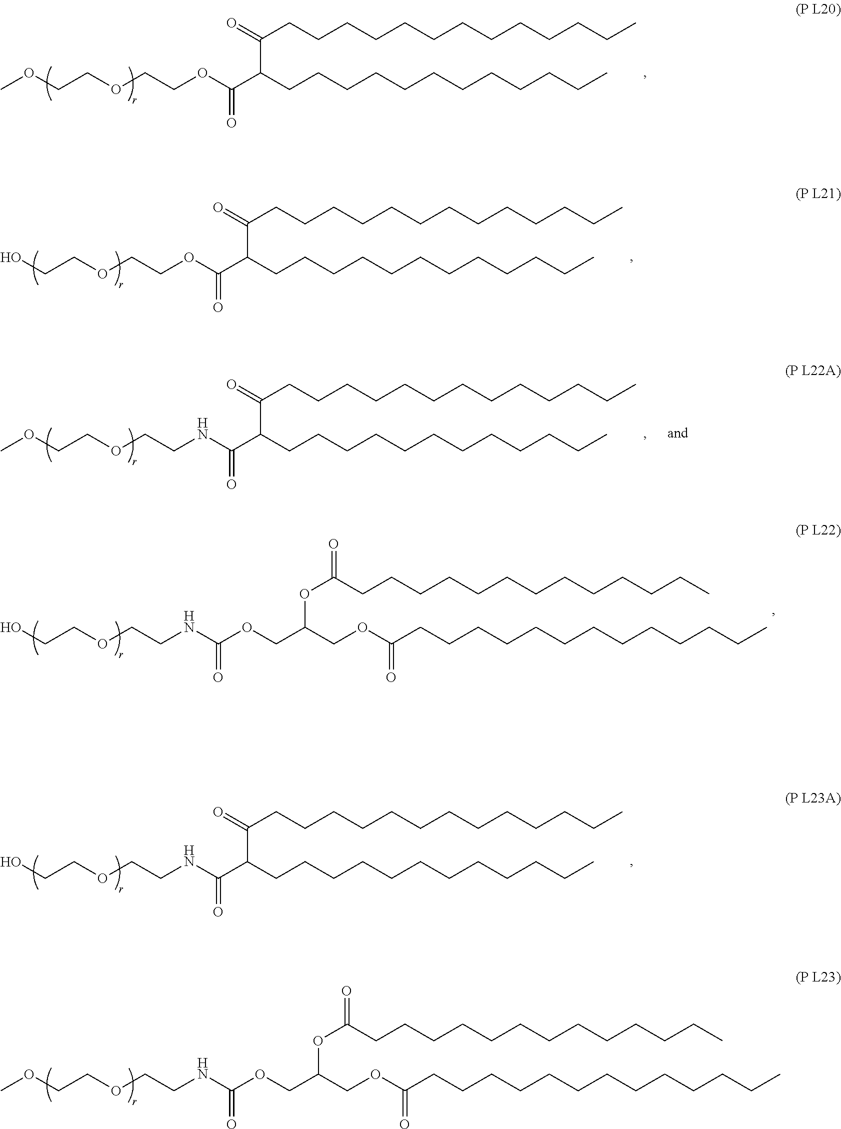

and pharmaceutically acceptable salts thereof.

In another aspect, provided herein are lipid nanoparticles (LNPs) comprising PEG lipids of Formula (PVIII):

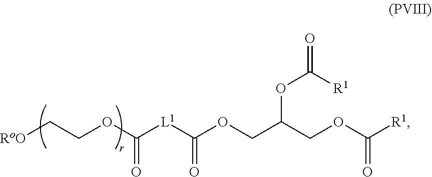

or pharmaceutically acceptable salts thereof, wherein:

L¹ is a bond, optionally substituted C₁₋₃ alkylene, optionally substituted C₁₋₃ heteroalkylene, optionally substituted C₂-3 alkenylene, optionally substituted C₂-3 alkynylene;

each instance of R₁ is independently optionally substituted C₅₋₃₀ alkyl, optionally substituted C₃-30 alkenyl, or optionally substituted C₅₋₃₀ alkynyl;

R^{O} is hydrogen, optionally substituted alkyl, optionally substituted acyl, or an oxygen protecting group;

r is an integer from 2 to 100, inclusive;

provided that when L¹ is —CH₂CH₂— or —CH₂CH₂CH₂—, R^{O} is not methyl.

In certain embodiments, when L¹ is optionally substituted C₂ or C₃ alkylene, R^{O} is not optionally substituted alkyl. In certain embodiments, when L¹ is optionally substituted C₂ or C₃ alkylene, R^{O} is hydrogen. In certain embodiments, when L¹ is —CH₂CH₂— or —CH₂CH₂CH₂—, R^{O} is not optionally substituted alkyl. In certain embodiments, when L′ is —CH₂CH₂— or —CH₂CH₂CH₂—, R^{O} is hydrogen.

In certain embodiments, the PEG lipid of Formula (PVIII) is of the formula:

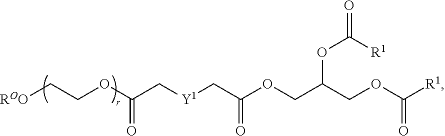

or a pharmaceutically acceptable salt thereof, wherein:

Y¹ is a bond, —CR₂—, —O—, —NR^{N}—, or —S—;

each instance of R is independently hydrogen, halogen, or optionally substituted alkyl;

R^{N} is hydrogen, optionally substituted alkyl, optionally substituted acyl, or a nitrogen protecting group;

provided that when Y¹ is a bond or —CH₂—, R^{O} is not methyl.

In certain embodiments, when L¹ is —CR₂—, R^{O} is not optionally substituted alkyl. In certain embodiments, when L¹ is —CR₂—, R^{O} is hydrogen. In certain embodiments, when L¹ is —CH₂—, R^{O} is not optionally substituted alkyl. In certain embodiments, when L¹ is —CH₂—, R^{O} is hydrogen.

In certain embodiments, the PEG lipid of Formula (PVIII) is of one of the following formulae:

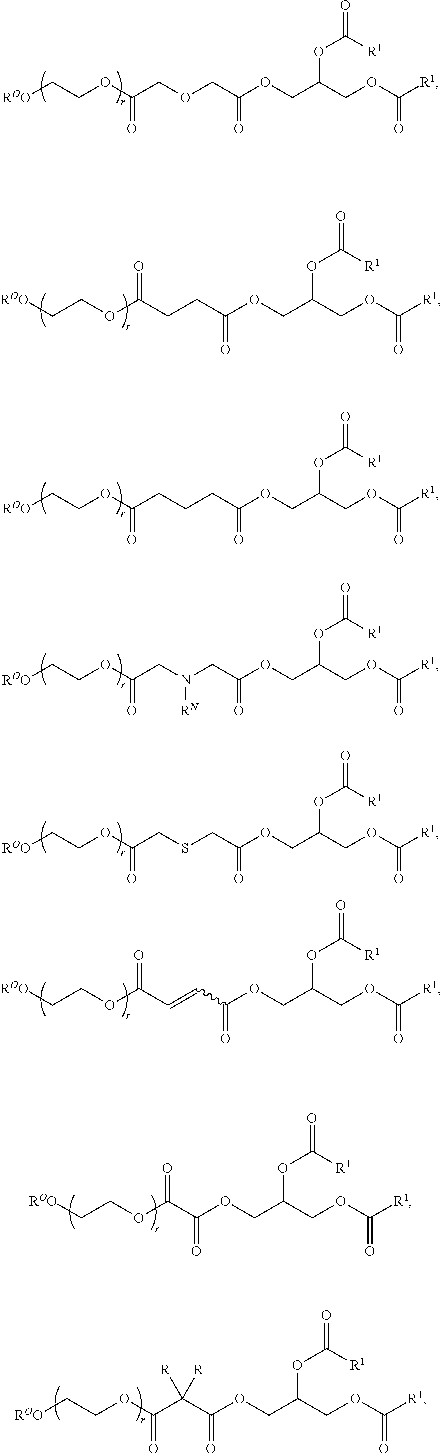

or a pharmaceutically acceptable salt thereof, wherein:

each instance of R is independently hydrogen, halogen, or optionally substituted alkyl.

In certain embodiments, the PEG lipid of Formula (PVIII) is of one of the following formulae:

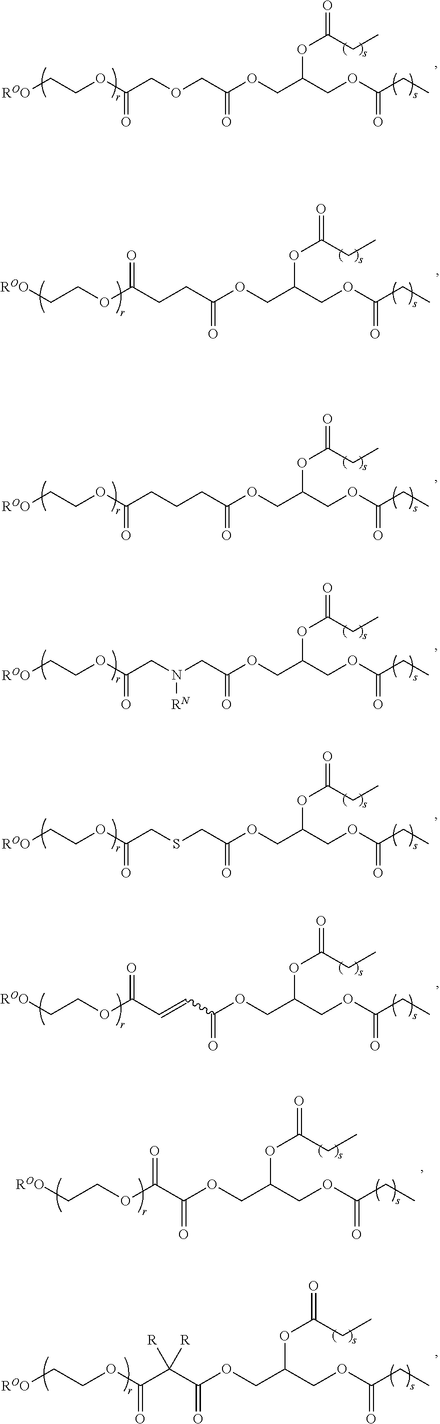

or a pharmaceutically acceptable salt thereof; wherein:

each instance of R is independently hydrogen, halogen, or optionally substituted alkyl; and

each s is independently an integer from 5-25, inclusive.

In certain embodiments, the PEG lipid of Formula (PVIII) is of one of the following formulae:

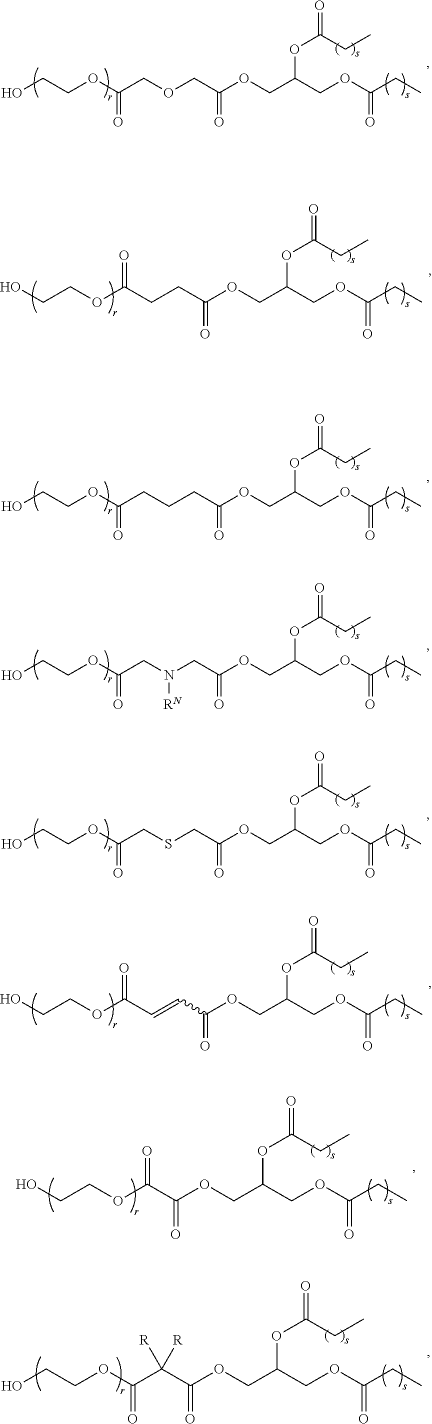

or a pharmaceutically acceptable salt thereof.

In certain embodiments, the PEG lipid of Formula (PVIII) is selected from the group consisting of:

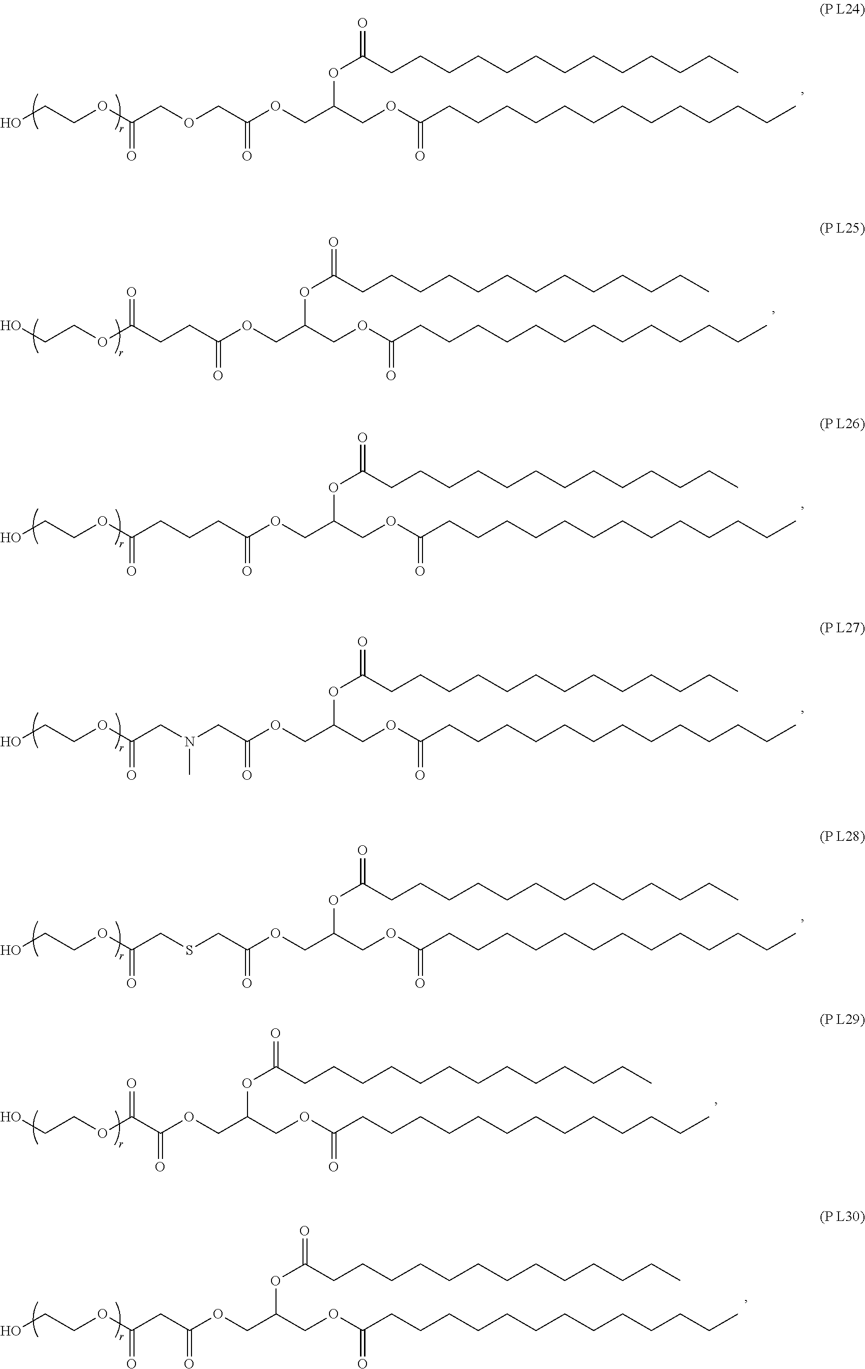

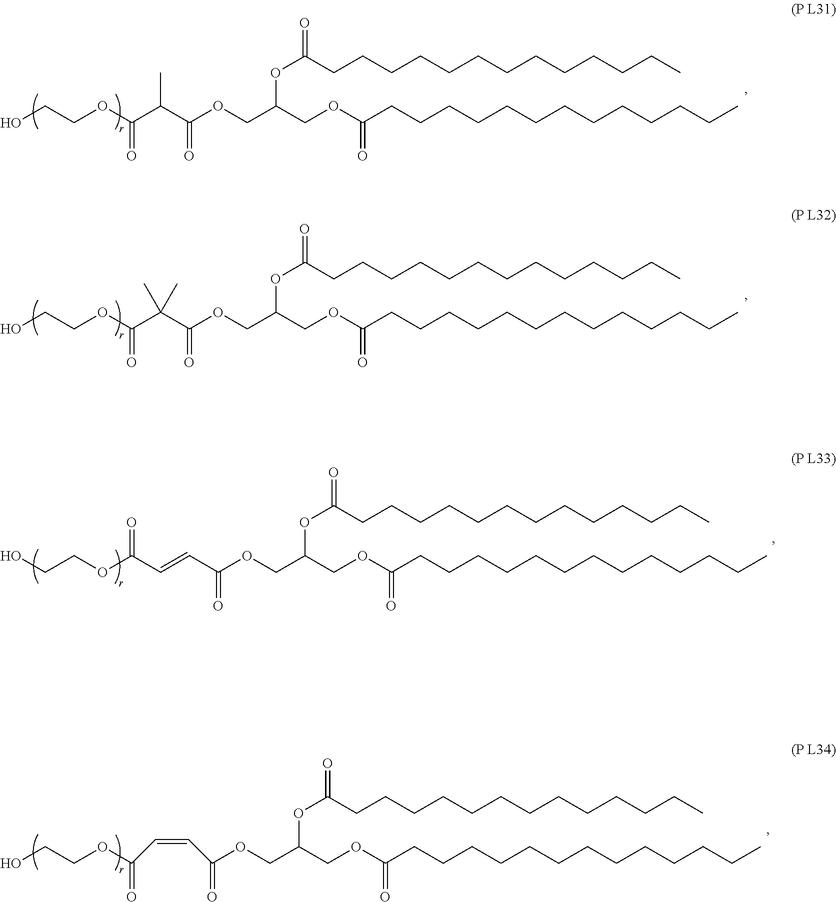

and pharmaceutically acceptable salts thereof.

In any of the foregoing or related aspects, a PEG lipid of the invention is featured wherein r is 40-50.

The LNPs provided herein, in certain embodiments, exhibit increased PEG shedding compared to existing LNP formulations comprising PEG lipids. “PEG shedding,” as used herein, refers to the cleavage of a PEG group from a PEG lipid. In many instances, cleavage of a PEG group from a PEG lipid occurs through serum-driven esterase-cleavage or hydrolysis. The PEG lipids provided herein, in certain embodiments, have been designed to control the rate of PEG shedding. In certain embodiments, an LNP provided herein exhibits greater than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits greater than 50% PEG shedding after about 6 hours in human serum. In certain embodiments, an LNP provided herein exhibits greater than 60% PEG shedding after about 6 hours in human serum. In certain embodiments, an LNP provided herein exhibits greater than 70% PEG shedding after about 6 hours in human serum. In certain embodiments, the LNP exhibits greater than 80% PEG shedding after about 6 hours in human serum. In certain embodiments, the LNP exhibits greater than 90% PEG shedding after about 6 hours in human serum. In certain embodiments, an LNP provided herein exhibits greater than 90% PEG shedding after about 6 hours in human serum.

In other embodiments, an LNP provided herein exhibits less than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum. In certain embodiments, an LNP provided herein exhibits less than 60% PEG shedding after about 6 hours in human serum. In certain embodiments, an LNP provided herein exhibits less than 70% PEG shedding after about 6 hours in human serum. In certain embodiments, an LNP provided herein exhibits less than 80% PEG shedding after about 6 hours in human serum.

In addition to the PEG lipids provided herein, the LNP may comprise one or more additional lipid components. In certain embodiments, the PEG lipids are present in the LNP in a molar ratio of 0.15-15% with respect to other lipids. In certain embodiments, the PEG lipids are present in a molar ratio of 0.15-5% with respect to other lipids. In certain embodiments, the PEG lipids are present in a molar ratio of 1-5% with respect to other lipids. In certain embodiments, the PEG lipids are present in a molar ratio of 0.15-2% with respect to other lipids. In certain embodiments, the PEG lipids are present in a molar ratio of 1-2% with respect to other lipids. In certain embodiments, the PEG lipids are present in a molar ratio of approximately 1%, 1.1%, 1.2%, 1.3%, 1.4%, 1.5%, 1.6%, 1.7%, 1.8%, 1.9%, or 2% with respect to other lipids. In certain embodiments, the PEG lipids are present in a molar ratio of approximately 1.5% with respect to other lipids.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

### Exemplary Synthesis

### Compound: HO-PEG₂₀₀₀-ester-C18

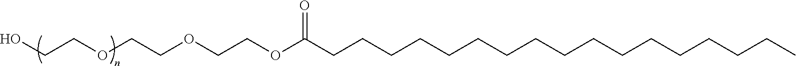

To a nitrogen filled flask containing palladium on carbon (10 wt. %, 74 mg, 0.070 mmol) was added Benzyl-PEG₂₀₀₀-ester-C18 (822 mg, 0.35 mmol) and MeOH (20 mL). The flask was evacuated nad backfilled with H₂ three times, and allowed to stir at RT and 1 atm H₂ for 12 hours. The mixture was filtered through celite, rinsing with DCM, and the filtrate was concentrated in vacuo to provide the desired product (692 mg, 88%). Using this methodology n=40-50. In one embodiment, n of the resulting polydispersed mixture is referred to by the average, 45.

For example, the value of r can be determined on the basis of a molecular weight of the PEG moiety within the PEG lipid. For example, a molecular weight of 2,000 (e.g., PEG2000) corresponds to a value of n of approximately 45. For a given composition, the value for n can connote a distribution of values within an art-accepted range, since polymers are often found as a distribution of different polymer chain lengths. For example, a skilled artisan understanding the polydispersity of such polymeric compositions would appreciate that an n value of 45 (e.g., in a structural formula) can represent a distribution of values between 40-50 in an actual PEG-containing composition, e.g., a DMG PEG200 peg lipid composition.

In some aspects, an LNP of the pharmaceutical compositions disclosed herein does not comprise a PEG-lipid.

In one embodiment, an LNP of the disclosure comprises a PEG-lipid. In one embodiment, the PEG lipid is not PEG DMG. In some aspects, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some aspects, the PEG lipid is selected from the group consisting of PEG-c-DOMG, PEG-DMG, PEG-DLPE, PEG-DMPE, PEG-DPPC and PEG-DSPE lipid. In other aspects, the PEG-lipid is PEG-DMG.

In one embodiment, an LNP of the disclosure comprises a PEG-lipid which has a chain length longer than about 14 or than about 10, if branched.

In one embodiment, the PEG lipid is a compound selected from the group consisting of any of Compound Nos. P415, P416, P417, P 419, P 420, P 423, P 424, P 428, P L¹, P L2, P L¹⁶, P L¹⁷, P L¹⁸, P L¹⁹, P L22 and P L23. In one embodiment, the PEG lipid is a compound selected from the group consisting of any of Compound Nos. P415, P417, P 420, P 423, P 424, P 428, P L¹, P L2, P L¹⁶, P L¹⁷, P L¹⁸, P L¹⁹, P L22 and P L23.

In one embodiment, a PEG lipid is selected from the group consisting of: Cmpd 428, PL¹⁶, PL¹⁷, PL 18, PL¹⁹, PL 1, and PL 2.

Exemplary LNP Lipids

In any of the foregoing or related aspects, the ionizable lipid (denoted by I) of the LNP of the disclosure comprises a compound included in any e.g. a compound having any of Formula (I I), (I IA), (IIB), (I II), (I Ha), (I IIb), (I IIc), (I IId), (IIe), (I III), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8) and/or any of Compounds X, Y, I 48, I 50, I 109, I 111, I 113, I 181, I 182, I 244, I 292, I 301, I 321, I 322, I 326, I 328, I 330, I 331, I 332 or I M.

In any of the foregoing or related aspects, the ionizable lipid of the LNP of the disclosure comprises a compound described herein as Compound X, Compound Y, Compound 1-321, Compound 1-292, Compound 1-326, Compound 1-182, Compound 1-301, Compound 1-48, Compound I-50, Compound 1-328, Compound 1-330, Compound 1-109, Compound I-111 or Compound I-181.

In any of the foregoing or related aspects, the ionizable lipid of the LNP of the disclosure comprises at least one compound selected from the group consisting of: Compound Nos. I 18 (also referred to as Compound X), I 25 (also referred to as Compound Y), I 48, I 50, I 109, I 111, I 113, I 181, I 182, I 244, I 292, I 301, I 309, I 317, I 321, I 322, I 326, I 328, I 330, I 331, I 332, I 347, I 348, I 349, I 350, I 351 and I 352. In another embodiment, the ionizable lipid of the LNP of the disclosure comprises a compound selected from the group consisting of: Compound Nos. I 18 (also referred to as Compound X), I 25 (also referred to as Compound Y), I 48, I 50, I 109, I 111, I 181, I 182, I 292, I 301, I 321, I 326, I 328, and I 330. In another embodiment, the ionizable lipid of the LNP of the disclosure comprises a compound selected from the group consisting of: Compound Nos. I 182, I 301, I 321, and I 326.

In one embodiment, a blend of ionizable lipids may be employed.

In one embodiment, an LNP comprises a sterol. In another embodiment, an LNP comprises a naturally occurring sterol. In another embodiment, an LNP comprises a modified sterol. In one embodiment, an LNP comprises one or more phytosterols. In one embodiment, an LNP comprises a phytosterol/cholesterol blend.

The term “phytosterol” refers to the group of plant based sterols and stanols that are phytosteroids including salts or esters thereof.

The term “sterol” refers to the subgroup of steroids also known as steroid alcohols. Sterols are usually divided into two classes: (1) plant sterols also known as “phytosterols”, and (2) animal sterols also known as “zoosterols” such as cholesterol. The term “stanol” refers to the class of saturated sterols, having no double bonds in the sterol ring structure.

In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

In some embodiments, the sitosterol is a beta-sitosterol.

In some embodiments, the beta-sitosterol has the formula:

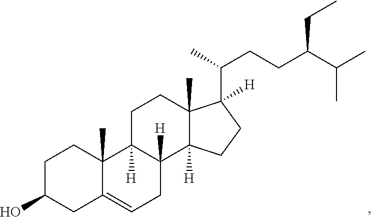

including analogs, salts or esters thereof.

In some embodiments, the sitosterol is a stigmasterol.

In some embodiments, the stigmasterol has the formula:

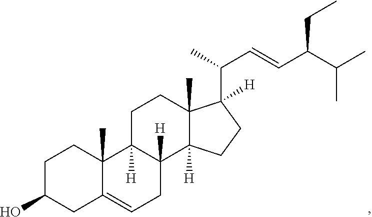

including analogs, salts or esters thereof.

In some embodiments, the sitosterol is a campesterol.

In some embodiments, the campesterol has the formula:

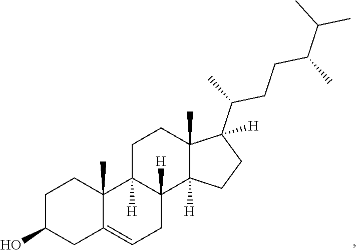

including analogs, salts or esters thereof.

In some embodiments, the sitosterol is a sitostanol.

In some embodiments, the sitostanol has the formula:

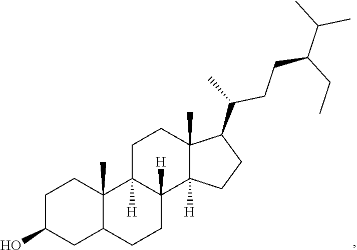

including analogs, salts or esters thereof.

In some embodiments, the sitosterol is a campestanol.

In some embodiments, the campestanol has the formula:

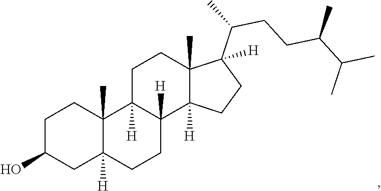

including analogs, salts or esters thereof.

In some embodiments, the sitosterol is a brassicasterol.

In some embodiments, the brassicasterol has the formula:

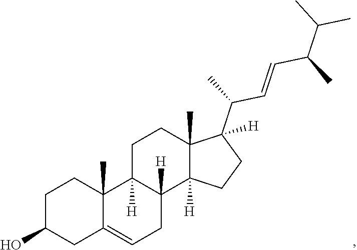

including analogs, salts or esters thereof.

In some embodiments, the sitosterol is a fucosterol.

In some embodiments, the fucosterol has the formula:

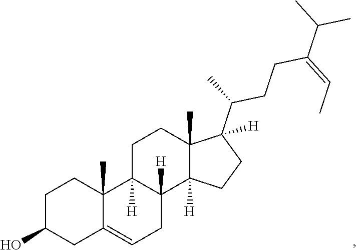

including analogs, salts or esters thereof.

In some embodiments, the phytosterol (e.g., beta-sitosterol) has a purity of greater than 70%. In some embodiments, the phytosterol (e.g., beta-sitosterol) has a purity of greater than 80%. In some embodiments, the phytosterol (e.g., beta-sitosterol) has a purity of greater than 90%. In some embodiments, the phytosterol (e.g., beta-sitosterol) has a purity of greater than 95%. In some embodiments, the phytosterol (e.g., beta-sitosterol) has a purity of greater than 97%, 98% or 99%.

In one embodiment, an LNP comprises more than one type of structural lipid.

For example, in one embodiment, the LNP comprises a phytosterol. In one embodiment, the phytosterol is the only structural lipid present in the LNP. In another embodiment, the LNP comprises a blend of structural lipids.

In one embodiment, the combined amount of the phytosterol and structural lipid (e.g., beta-sitosterol and cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one embodiment, the combined amount of the phytosterol and structural lipid (e.g., beta-sitosterol and cholesterol) in the lipid composition disclosed herein ranges from about 25 mol % to about 30 mol %, from about 30 mol % to about 35 mol %, or from about 35 mol % to about 40 mol %.

In one embodiment, the amount of the phytosterol and structural lipid (e.g., beta-sitosterol and cholesterol) in the lipid composition disclosed herein is about 24 mol %, about 29 mol %, about 34 mol %, or about 39 mol %.

In some embodiments, the combined amount of the phytosterol and structural lipid (e.g., beta-sitosterol and cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

In some embodiments, the lipid nanoparticle comprises one or more phytosterols (e.g., beta-sitosterol) and one or more structural lipids (e.g. cholesterol). In some embodiments, the mol % of the structural lipid is between about 1% and 50% of the mol % of phytosterol present in the lipid nanoparticle. In some embodiments, the mol % of the structural lipid is between about 10% and 40% of the mol % of phytosterol present in the lipid-based composition (e.g., LNP). In some embodiments, the mol % of the structural lipid is between about 20% and 30% of the mol % of phytosterol present in the lipid-based composition (e.g., LNP). In some embodiments, the mol % of the structural lipid is about 30% of the mol % of phytosterol present in the lipid-based composition (e.g., lipid nanoparticle).

In some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17,18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

In certain embodiments, the LNP comprises 50% ionizable lipid, 10% helper lipid (e.g, phospholipid), 38.5% structural lipid, and 1.5% PEG lipid. In certain embodiments, the LNP comprises 50% ionizable lipid, 10% helper lipid (e.g, phospholipid), 38% structural lipid, and 2% PEG lipid. In certain embodiments, the LNP comprises 50% ionizable lipid, 20% helper lipid (e.g, phospholipid), 28.5% structural lipid, and 1.5% PEG lipid. In certain embodiments, the LNP comprises 50% ionizable lipid, 20% helper lipid (e.g, phospholipid), 28% structural lipid, and 2% PEG lipid. In certain embodiments, the LNP comprises 40% ionizable lipid, 30% helper lipid (e.g, phospholipid), 28.5% structural lipid, and 1.5% PEG lipid. In certain embodiments, the LNP comprises 40% ionizable lipid, 30% helper lipid (e.g, phospholipid), 28% structural lipid, and 2% PEG lipid. In certain embodiments, the LNP comprises 45% ionizable lipid, 20% helper lipid (e.g, phospholipid), 33.5% structural lipid, and 1.5% PEG lipid. In certain embodiments, the LNP comprises 45% ionizable lipid, 20% helper lipid (e.g, phospholipid), 33% structural lipid, and 2% PEG lipid.

In one aspect, the LNP comprises phytosterol and the LNP does not comprise an additional structural lipid. Accordingly, the structural lipid (sterol) component of the LNP consists of phytosterol. In another aspect, the LNP comprises phytosterol and an additional structural lipid. Accordingly, the sterol component of the LNP comprise phytosterol and one or more additional sterols or structural lipids.

In any of the foregoing or related aspects, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), brassicasterol or stigmasterol, or combinations or blends thereof. In another embodiment, the structural lipid (e.g., sterol, such as a phytosterol or phytosterol/cholesterol blend) of the LNP of the disclosure comprises a compound selected from cholesterol, β-sitosterol, campesterol, β-sitostanol, brassicasterol, stigmasterol, β-sitosterol-d7, Compound S-30, Compound S-31, Compound S-32, or combinations or blends thereof. In another embodiment, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), Compound S-140, Compound S-144, brassicasterol (also referred to herein as Cmpd S 148) or Composition S-183 (˜40% Compound S-141, ˜25% Compound S-140, ˜25% Compound S-143 and ˜10% brassicasterol). In some embodiments, the structural lipid of the LNP of the disclosure comprises a compound described herein as Compound S-159, Compound S-160, Compound S-164, Compound S-165, Compound S-167, Compound S-170, Compound S-173 or Compound S-175.

In one embodiment, an LNP comprises a non-cationic helper lipid, e.g., phospholipid. In any of the foregoing or related aspects, the non-cationic helper lipid (e.g, phospholipid) of the LNP of the disclosure comprises a compound described herein as DSPC, DMPE, DOPC or H-409. In one embodiment, the non-cationic helper lipid, e.g., phospholipid is DSPC. In other embodiments, the non-cationic helper lipid (e.g., phospholipid) of the LNP of the disclosure comprises a compound described herein as DSPC, DMPE, DOPC, DPPC, PMPC, H-409, H-418, H-420, H-421 or H-422.

In any of the foregoing or related aspects, the PEG lipid of the LNP of the disclosure comprises a compound described herein can be selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In another embodiment, the PEG lipid is selected from the group consisting of Compound Nos. P415, P416, P417, P 419, P 420, P 423, P 424, P 428, P L5, P L¹, P L2, P L¹⁶, P L¹⁷, P L¹⁸, P L¹⁹, P L22, P L23, DMG, DPG and DSG. In another embodiment, the PEG lipid is selected from the group consisting of Cmpd 428, PL¹⁶, PL¹⁷, PL 18, PL¹⁹, P L5, PL 1, and PL 2.

In other embodiments, the disclosure provides lipid nanoparticles comprising Compound X as the ionizable lipid, DSPC as the phospholipid, cholesterol or a cholesterol/β-sitosterol blend as the structural lipid and Compound 428 as the PEG lipid. In various embodiments of these Compound X-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol. In another embodiment, the structural lipid is cholesterol/β-sitosterol at a total percentage of 38.5%, wherein the blend can comprise, for example: (i) 20% cholesterol and 18.5% (3-sitosterol; or (ii) 10% cholesterol and 28.5% β-sitosterol.

In other embodiments, the disclosure provides lipid nanoparticles comprising any of Compounds X, Y, 1-321, 1-292, 1-326, 1-182, 1-301, 1-48, I-50, 1-328, 1-330, 1-109, I-111 or I-181 as the ionizable lipid; DSPC as the phospholipid; cholesterol, a cholesterol/β-sitosterol blend, a β-sitosterol/β-sitostanol blend, a β-sitosterol/camposterol blend, a β-sitosterol/β-sitostanol/camposterol blend, a cholesterol/camposterol blend, a cholesterol/β-sitostanol blend, a cholesterol/β-sitostanol/camposterol blend or a cholesterol/β-sitosterol/β-sitostanol/camposterol blend as the structural lipid; and Compound 428 as the PEG lipid. In various embodiments of these compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; (v) 40:18.5:40:1.5; or (vi) 45:20:33.5:1.5. In one embodiment, for the structural lipid component, the LNP can comprise, for example, 40% structural lipid composed of (i) 10% cholesterol and 30% β-sitosterol; (ii) 10% cholesterol and 30% campesterol; (iii) 10% cholesterol and 30% β-sitostanol; (iv) 10% cholesterol, 20% β-sitosterol and 10% campesterol; (v) 10% cholesterol, 20% β-sitosterol and 10% β-sitostanol; (vi) 10% cholesterol, 10% β-sitosterol and 20% campesterol; (vii) 10% cholesterol, 10% β-sitosterol and 20% campesterol; (viii) 10% cholesterol, 20% campesterol and 10% (3-sitostanol; (ix) 10% cholesterol, 10% campesterol and 20% β-sitostanol; or (x) 10% cholesterol, 10% β-sitosterol, 10% campesterol and 10% β-sitostanol. In another embodiment, for the structural lipid component, the LNP can comprise, for example, 33.5% structural lipid composed of (i) 33.5% cholesterol; (ii) 18.5% cholesterol, 15% β-sitosterol; (iii) 18.5% cholesterol, 15% campesterol; or (iv) 18.5% cholesterol, 15% campesterol.

In other embodiment, the disclosure provides lipid nanoparticles comprising camposterol, β-sitostanol or stigmasterol as the structural lipid. The other components of the LNP can be selected from those disclosed herein, for example Compound X, Compound I-109, Compound I-111, Compound 1-181, Compound 1-182 or Compound 1-244 as the ionizable lipid; DSPC as the phospholipid; and Compound 428 as the PEG lipid.

Exemplary Additional LNP Components Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer. For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10⁻⁴ M in water at about 30° C. and atmospheric pressure.

For example, amphiphilic polymer has a critical micelle concentration (CMC) ranging between about 0.1×10⁻⁴ M and about 1.3×10⁻⁴ M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

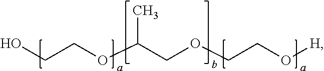

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof.

For example, the polyethylene glycol ether is a compound of Formula (VIII):

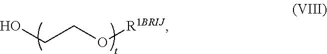

or a salt or isomer thereof, wherein:

t is an integer between 1 and 100;

R^{iBRIJ} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, S, C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NRNC(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group

In some embodiment, R^{iBRIJ} is C₁₈ alkyl. For example, the polyethylene glycol ether is a compound of Formula (VIII-a):

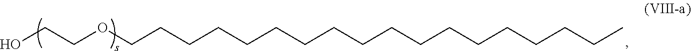

or a salt or isomer thereof.

In some embodiments, R^{iBRIJ} is C₁₈ alkenyl. For example, the polyethylene glycol ether is a compound of Formula (VIII-b):

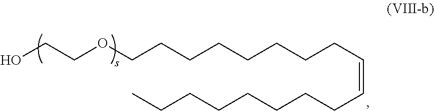

or a salt or isomer thereof

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

Adjuvants

In some embodiments, an LNP of the invention optionally includes one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

Other Components

An LNP of the invention may optionally include one or more components in addition to those described in the preceding sections. For example, a lipid nanoparticle may include one or more small hydrophobic molecules such as a vitamin (e.g., vitamin A or vitamin E) or a sterol.

Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21St Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

LNP Compositions

A lipid nanoparticle described herein may be designed for one or more specific applications or targets. The elements of a lipid nanoparticle and their relative amounts may be selected based on a particular application or target, and/or based on the efficacy, toxicity, expense, ease of use, availability, or other feature of one or more elements. Similarly, the particular formulation of a lipid nanoparticle may be selected for a particular application or target according to, for example, the efficacy and toxicity of particular combinations of elements. The efficacy and tolerability of a lipid nanoparticle formulation may be affected by the stability of the formulation.

The elements of the various components may be provided in specific fractions, e.g., mole percent fractions.

For example, in any of the foregoing or related aspects, the LNP of the disclosure comprises a structural lipid or a salt thereof. In some aspects, the structural lipid is cholesterol or a salt thereof. In further aspects, the mol % cholesterol is between about 1% and 50% of the mol % of phytosterol present in the LNP. In other aspects, the mol % cholesterol is between about 10% and 40% of the mol % of phytosterol present in the LNP. In some aspects, the mol % cholesterol is between about 20% and 30% of the mol % of phytosterol present in the LNP. In further aspects, the mol % cholesterol is about 30% of the mol % of phytosterol present in the LNP.

In any of the foregoing or related aspects, the LNP of the disclosure comprises about 30 mol % to about 60 mol % ionizable lipid, about 0 mol % to about 30 mol % phospholipid, about 18.5 mol % to about 48.5 mol % sterol, and about 0 mol % to about 10 m of % PEG lipid.

In any of the foregoing or related aspects, the LNP of the disclosure comprises about 35 mol % to about 55 mol % ionizable lipid, about 5 mol % to about 25 mol % phospholipid, about 30 mol % to about 40 mol % sterol, and about 0 mol % to about 10 mol % PEG lipid.

In any of the foregoing or related aspects, the LNP of the disclosure comprises about 50 mol % ionizable lipid, about 10 mol % phospholipid, about 38.5 mol % sterol, and about 1.5 mol % PEG lipid.

In certain embodiments, the ionizable lipid component of the lipid nanoparticle includes about 30 mol % to about 60 mol % ionizable lipid, about 0 mol % to about 30 mol % non-cationic helper lipid, about 18.5 mol % to about 48.5 mol % phytosterol optionally including one or more structural lipids, and about 0 mol % to about 10 mol % of PEG lipid, provided that the total mol % does not exceed 100%. In some embodiments, the ionizable lipid component of the lipid nanoparticle includes about 35 mol % to about 55 mol % ionizable lipid, about 5 mol % to about 25 mol % non-cationic helper lipid, about 30 mol % to about 40 mol % phytosterol optionally including one or more structural lipids, and about 0 mol % to about 10 mol % of PEG lipid. In a particular embodiment, the lipid component includes about 50 mol % ionizable lipid, about 10 mol % non-cationic helper lipid, about 38.5 mol % phytosterol optionally including one or more structural lipids, and about 1.5 mol % of PEG lipid. In another particular embodiment, the lipid component includes about 40 mol % ionizable lipid, about 20 mol % non-cationic helper lipid, about 38.5 mol % phytosterol optionally including one or more structural lipids, and about 1.5 mol % of PEG lipid. In some embodiments, the phytosterol may be beta-sitosterol, the non-cationic helper lipid may be a phospholipid such as DOPE, DSPC or a phospholipid substitute such as oleic acid. In other embodiments, the PEG lipid may be PEG-DMG and/or the structural lipid may be cholesterol.

In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and a structural lipid, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and cholesterol, and about 0 mol % to about 10 mol % PEG lipid.

In some aspects, the LNP of the disclosure comprises about 35 mol % to about 55 mol % ionizable lipid, about 5 mol % to about 25 mol % non-cationic helper lipid, about 30 mol % to about 40 mol % phytosterol, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and a structural lipid, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and cholesterol, and about 0 mol % to about 10 mol % PEG lipid.

In some aspects, the LNP of the disclosure comprises about 50 mol % ionizable lipid, about 10 mol % non-cationic helper lipid, about 38.5 mol % phytosterol, and about 1.5 mol % PEG lipid. In some aspects, the LNP of the disclosure comprises about 50 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 50 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 38.5 mol % phytosterol, and about 1.5 mol % PEG lipid. In some aspects, the LNP of the disclosure comprises about 40 mol % ionizable lipid, about 20 mol % non-cationic helper lipid, about 38.5 mol % phytosterol and a structural lipid, and about 1.5 mol % PEG lipid. In some aspects, the LNP of the disclosure comprises about 40 mol % ionizable lipid, about 20 mol % non-cationic helper lipid, about 38.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 38.5 mol % phytosterol, and about 1.5 mol % PEG lipid. In some aspects, the LNP of the disclosure comprises about 45 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 55 mol % ionizable lipid, about 5 mol % non-cationic helper lipid, about 38.5 mol % phytosterol, and about 1.5 mol % PEG lipid. In some aspects, the LNP of the disclosure comprises about 55 mol % ionizable lipid, about 5 mol % non-cationic helper lipid, about 38.5 mol % phytosterol and a structural lipid, and about 1.5 mol % PEG lipid. In some aspects, the LNP of the disclosure comprises about 55 mol % ionizable lipid, about 5 mol % non-cationic helper lipid, about 38.5 mol % phytosterol and cholesterol, and about 1.5 mol % PEG lipid.

In some aspects, the LNP of the disclosure comprises about 60 mol % ionizable lipid, about 5 mol % non-cationic helper lipid, about 33.5 mol % phytosterol, and about 1.5 mol % PEG lipid. In some aspects, the LNP of the disclosure comprises about 60 mol % ionizable lipid, about 5 mol % non-cationic helper lipid, about 33.5 mol % phytosterol and a structural lipid, and about 1.5 mol % PEG lipid. In some aspects, the LNP of the disclosure comprises about 60 mol % ionizable lipid, about 5 mol % non-cationic helper lipid, about 33.5 mol % phytosterol and cholesterol, and about 1.5 mol % PEG lipid.

In some aspects, the LNP of the disclosure comprises about 45 mol % ionizable lipid, about 20 mol % non-cationic helper lipid, about 33.5 mol % phytosterol, and about 1.5 mol % PEG lipid. In some aspects, the LNP of the disclosure comprises about 45 mol % ionizable lipid, about 20 mol % non-cationic helper lipid, about 33.5 mol % phytosterol and a structural lipid, and about 1.5 mol % PEG lipid. In some aspects, the LNP of the disclosure comprises about 45 mol % ionizable lipid, about 20 mol % non-cationic helper lipid, about 33.5 mol % phytosterol and cholesterol, and about 1.5 mol % PEG lipid.

In some aspects, the LNP of the disclosure comprises about 50 mol % ionizable lipid, about 20 mol % non-cationic helper lipid, about 28.5 mol % phytosterol, and about 1.5 mol % PEG lipid. In some aspects, the LNP of the disclosure comprises about 50 mol % ionizable lipid, about 20 mol % non-cationic helper lipid, about 28.5 mol % phytosterol and a structural lipid, and about 1.5 mol % PEG lipid. In some aspects, the LNP of the disclosure comprises about 50 mol % ionizable lipid, about 20 mol % non-cationic helper lipid, about 28.5 mol % phytosterol and cholesterol, and about 1.5 mol % PEG lipid.

In some aspects, the LNP of the disclosure comprises about 55 mol % ionizable lipid, about 20 mol % non-cationic helper lipid, about 23.5 mol % phytosterol, and about 1.5 mol % PEG lipid. In some aspects, the LNP of the disclosure comprises about 55 mol % ionizable lipid, about 20 mol % non-cationic helper lipid, about 23.5 mol % phytosterol and a structural lipid, and about 1.5 mol % PEG lipid. In some aspects, the LNP of the disclosure comprises about 55 mol % ionizable lipid, about 20 mol % non-cationic helper lipid, about 23.5 mol % phytosterol and cholesterol, and about 1.5 mol % PEG lipid.

In some aspects, the LNP of the disclosure comprises about 60 mol % ionizable lipid, about 20 mol % non-cationic helper lipid, about 18.5 mol % phytosterol, and about 1.5 mol % PEG lipid. In some aspects, the LNP of the disclosure comprises about 60 mol % ionizable lipid, about 20 mol % non-cationic helper lipid, about 18.5 mol % phytosterol and a structural lipid, and about 1.5 mol % PEG lipid. In some aspects, the LNP of the disclosure comprises about 60 mol % ionizable lipid, about 20 mol % non-cationic helper lipid, about 18.5 mol % phytosterol and cholesterol, and about 1.5 mol % PEG lipid.

In some aspects, the LNP of the disclosure comprises about 40 mol % ionizable lipid, about 15 mol % non-cationic helper lipid, about 43.5 mol % phytosterol, and about 1.5 mol % PEG lipid. In some aspects, the LNP of the disclosure comprises about 40 mol % ionizable lipid, about 15 mol % non-cationic helper lipid, about 43.5 mol % phytosterol and a structural lipid, and about 1.5 mol % PEG lipid. In some aspects, the LNP of the disclosure comprises about 40 mol % ionizable lipid, about 15 mol % non-cationic helper lipid, about 43.5 mol % phytosterol and cholesterol, and about 1.5 mol % PEG lipid.

In some aspects, the LNP of the disclosure comprises about 50 mol % ionizable lipid, about 15 mol % non-cationic helper lipid, about 33.5 mol % phytosterol, and about 1.5 mol % PEG lipid. In some aspects, the LNP of the disclosure comprises about 50 mol % ionizable lipid, about 15 mol % non-cationic helper lipid, about 33.5 mol % phytosterol and a structural lipid, and about 1.5 mol % PEG lipid. In some aspects, the LNP of the disclosure comprises about 50 mol % ionizable lipid, about 15 mol % non-cationic helper lipid, about 33.5 mol % phytosterol and cholesterol, and about 1.5 mol % PEG lipid.

In some aspects, the LNP of the disclosure comprises about 55 mol % ionizable lipid, about 15 mol % non-cationic helper lipid, about 28.5 mol % phytosterol, and about 1.5 mol % PEG lipid. In some aspects, the LNP of the disclosure comprises about 55 mol % ionizable lipid, about 15 mol % non-cationic helper lipid, about 28.5 mol % phytosterol and a structural lipid, and about 1.5 mol % PEG lipid. In some aspects, the LNP of the disclosure comprises about 55 mol % ionizable lipid, about 15 mol % non-cationic helper lipid, about 28.5 mol % phytosterol and cholesterol, and about 1.5 mol % PEG lipid.

In some aspects, the LNP of the disclosure comprises about 60 mol % ionizable lipid, about 15 mol % non-cationic helper lipid, about 23.5 mol % phytosterol, and about 1.5 mol % PEG lipid. In some aspects, the LNP of the disclosure comprises about 60 mol % ionizable lipid, about 15 mol % non-cationic helper lipid, about 23.5 mol % phytosterol and a structural lipid, and about 1.5 mol % PEG lipid. In some aspects, the LNP of the disclosure comprises about 60 mol % ionizable lipid, about 15 mol % non-cationic helper lipid, about 23.5 mol % phytosterol and cholesterol, and about 1.5 mol % PEG lipid.

In some aspects, the LNP of the disclosure comprises about 40 mol % ionizable lipid, about 10 mol % non-cationic helper lipid, about 48.5 mol % phytosterol, and about 1.5 mol % PEG lipid. In some aspects, the LNP of the disclosure comprises about 40 mol % ionizable lipid, about 10 mol % non-cationic helper lipid, about 48.5 mol % phytosterol and a structural lipid, and about 1.5 mol % PEG lipid. In some aspects, the LNP of the disclosure comprises about 40 mol % ionizable lipid, about 10 mol % non-cationic helper lipid, about 48.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 43.5 mol % phytosterol, and about 1.5 mol % PEG lipid. In some aspects, the LNP of the disclosure comprises about 45 mol % ionizable lipid, about 10 mol % non-cationic helper lipid, about 43.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 43.5 mol % phytosterol and cholesterol, and about 1.5 mol % PEG lipid.

In some aspects, the LNP of the disclosure comprises about 55 mol % ionizable lipid, about 10 mol % non-cationic helper lipid, about 33.5 mol % phytosterol, and about 1.5 mol % PEG lipid. In some aspects, the LNP of the disclosure comprises about 55 mol % ionizable lipid, about 10 mol % non-cationic helper lipid, about 33.5 mol % phytosterol and a structural lipid, and about 1.5 mol % PEG lipid. In some aspects, the LNP of the disclosure comprises about 55 mol % ionizable lipid, about 10 mol % non-cationic helper lipid, about 33.5 mol % phytosterol and cholesterol, and about 1.5 mol % PEG lipid.

In some aspects, the LNP of the disclosure comprises about 60 mol % ionizable lipid, about 10 mol % non-cationic helper lipid, about 28.5 mol % phytosterol, and about 1.5 mol % PEG lipid. In some aspects, the LNP of the disclosure comprises about 60 mol % ionizable lipid, about 10 mol % non-cationic helper lipid, about 28.5 mol % phytosterol and a structural lipid, and about 1.5 mol % PEG lipid. In some aspects, the LNP of the disclosure comprises about 60 mol % ionizable lipid, about 10 mol % non-cationic helper lipid, about 28.5 mol % phytosterol and cholesterol, and about 1.5 mol % PEG lipid.

In some aspects, the LNP of the disclosure comprises about 40 mol % ionizable lipid, about 5 mol % non-cationic helper lipid, about 53.5 mol % phytosterol, and about 1.5 mol % PEG lipid. In some aspects, the LNP of the disclosure comprises about 40 mol % ionizable lipid, about 5 mol % non-cationic helper lipid, about 53.5 mol % phytosterol and a structural lipid, and about 1.5 mol % PEG lipid. In some aspects, the LNP of the disclosure comprises about 40 mol % ionizable lipid, about 5 mol % non-cationic helper lipid, about 53.5 mol % phytosterol and cholesterol, and about 1.5 mol % PEG lipid.

In some aspects, the LNP of the disclosure comprises about 45 mol % ionizable lipid, about 5 mol % non-cationic helper lipid, about 48.5 mol % phytosterol, and about 1.5 mol % PEG lipid. In some aspects, the LNP of the disclosure comprises about 45 mol % ionizable lipid, about 5 mol % non-cationic helper lipid, about 48.5 mol % phytosterol and a structural lipid, and about 1.5 mol % PEG lipid. In some aspects, the LNP of the disclosure comprises about 45 mol % ionizable lipid, about 5 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 5 mol % non-cationic helper lipid, about 43.5 mol % phytosterol, and about 1.5 mol % PEG lipid. In some aspects, the LNP of the disclosure comprises about 50 mol % ionizable lipid, about 5 mol % non-cationic helper lipid, about 43.5 mol % phytosterol and a structural lipid, and about 1.5 mol % PEG lipid. In some aspects, the LNP of the disclosure comprises about 50 mol % ionizable lipid, about 5 mol % non-cationic helper lipid, about 43.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 40 mol % phytosterol, and about 0 mol % PEG lipid. In some aspects, the LNP of the disclosure comprises about 40 mol % ionizable lipid, about 20 mol % non-cationic helper lipid, about 40 mol % phytosterol and a structural lipid, and about 0 mol % PEG lipid. In some aspects, the LNP of the disclosure comprises about 40 mol % ionizable lipid, about 20 mol % non-cationic helper lipid, about 40 mol % phytosterol and cholesterol, and about 0 mol % PEG lipid.

In some aspects, the LNP of the disclosure comprises about 45 mol % ionizable lipid, about 20 mol % non-cationic helper lipid, about 35 mol % phytosterol, and about 0 mol % PEG lipid. In some aspects, the LNP of the disclosure comprises about 45 mol % ionizable lipid, about 20 mol % non-cationic helper lipid, about 35 mol % phytosterol and a structural lipid, and about 0 mol % PEG lipid. In some aspects, the LNP of the disclosure comprises about 45 mol % ionizable lipid, about 20 mol % non-cationic helper lipid, about 35 mol % phytosterol and cholesterol, and about 0 mol % PEG lipid.

In some aspects, the LNP of the disclosure comprises about 50 mol % ionizable lipid, about 20 mol % non-cationic helper lipid, about 30 mol % phytosterol, and about 0 mol % PEG lipid. In some aspects, the LNP of the disclosure comprises about 50 mol % ionizable lipid, about 20 mol % non-cationic helper lipid, about 30 mol % phytosterol and a structural lipid, and about 0 mol % PEG lipid. In some aspects, the LNP of the disclosure comprises about 50 mol % ionizable lipid, about 20 mol % non-cationic helper lipid, about 30 mol % phytosterol and cholesterol, and about 0 mol % PEG lipid.

In some aspects, the LNP of the disclosure comprises about 55 mol % ionizable lipid, about 20 mol % non-cationic helper lipid, about 25 mol % phytosterol, and about 0 mol % PEG lipid. In some aspects, the LNP of the disclosure comprises about 55 mol % ionizable lipid, about 20 mol % non-cationic helper lipid, about 25 mol % phytosterol and a structural lipid, and about 0 mol % PEG lipid. In some aspects, the LNP of the disclosure comprises about 55 mol % ionizable lipid, about 20 mol % non-cationic helper lipid, about 25 mol % phytosterol and cholesterol, and about 0 mol % PEG lipid.

In some aspects, the LNP of the disclosure comprises about 60 mol % ionizable lipid, about 20 mol % non-cationic helper lipid, about 20 mol % phytosterol, and about 0 mol % PEG lipid. In some aspects, the LNP of the disclosure comprises about 60 mol % ionizable lipid, about 20 mol % non-cationic helper lipid, about 20 mol % phytosterol and a structural lipid, and about 0 mol % PEG lipid. In some aspects, the LNP of the disclosure comprises about 60 mol % ionizable lipid, about 20 mol % non-cationic helper lipid, about 20 mol % phytosterol and cholesterol, and about 0 mol % PEG lipid.

In some aspects, the LNP of the disclosure comprises about 40 mol % ionizable lipid, about 15 mol % non-cationic helper lipid, about 45 mol % phytosterol, and about 0 mol % PEG lipid. In some aspects, the LNP of the disclosure comprises about 40 mol % ionizable lipid, about 15 mol % non-cationic helper lipid, about 45 mol % phytosterol and a structural lipid, and about 0 mol % PEG lipid. In some aspects, the LNP of the disclosure comprises about 40 mol % ionizable lipid, about 15 mol % non-cationic helper lipid, about 45 mol % phytosterol and cholesterol, and about 0 mol % PEG lipid.

In some aspects, the LNP of the disclosure comprises about 45 mol % ionizable lipid, about 15 mol % non-cationic helper lipid, about 40 mol % phytosterol, and about 0 mol % PEG lipid. In some aspects, the LNP of the disclosure comprises about 45 mol % ionizable lipid, about 15 mol % non-cationic helper lipid, about 40 mol % phytosterol and a structural lipid, and about 0 mol % PEG lipid. In some aspects, the LNP of the disclosure comprises about 45 mol % ionizable lipid, about 15 mol % non-cationic helper lipid, about 40 mol % phytosterol and cholesterol, and about 0 mol % PEG lipid.

In some aspects, the LNP of the disclosure comprises about 50 mol % ionizable lipid, about 15 mol % non-cationic helper lipid, about 35 mol % phytosterol, and about 0 mol % PEG lipid. In some aspects, the LNP of the disclosure comprises about 50 mol % ionizable lipid, about 15 mol % non-cationic helper lipid, about 35 mol % phytosterol and a structural lipid, and about 0 mol % PEG lipid. In some aspects, the LNP of the disclosure comprises about 50 mol % ionizable lipid, about 15 mol % non-cationic helper lipid, about 35 mol % phytosterol and cholesterol, and about 0 mol % PEG lipid.

In some aspects, the LNP of the disclosure comprises about 55 mol % ionizable lipid, about 15 mol % non-cationic helper lipid, about 30 mol % phytosterol, and about 0 mol % PEG lipid. In some aspects, the LNP of the disclosure comprises about 55 mol % ionizable lipid, about 15 mol % non-cationic helper lipid, about 30 mol % phytosterol and a structural lipid, and about 0 mol % PEG lipid. In some aspects, the LNP of the disclosure comprises about 55 mol % ionizable lipid, about 15 mol % non-cationic helper lipid, about 30 mol % phytosterol and cholesterol, and about 0 mol % PEG lipid.

In some aspects, the LNP of the disclosure comprises about 60 mol % ionizable lipid, about 15 mol % non-cationic helper lipid, about 25 mol % phytosterol, and about 0 mol % PEG lipid. In some aspects, the LNP of the disclosure comprises about 60 mol % ionizable lipid, about 15 mol % non-cationic helper lipid, about 25 mol % phytosterol and a structural lipid, and about 0 mol % PEG lipid. In some aspects, the LNP of the disclosure comprises about 60 mol % ionizable lipid, about 15 mol % non-cationic helper lipid, about 25 mol % phytosterol and cholesterol, and about 0 mol % PEG lipid.

In some aspects, the LNP of the disclosure comprises about 40 mol % ionizable lipid, about 10 mol % non-cationic helper lipid, about 50 mol % phytosterol, and about 0 mol % PEG lipid. In some aspects, the LNP of the disclosure comprises about 40 mol % ionizable lipid, about 10 mol % non-cationic helper lipid, about 50 mol % phytosterol and a structural lipid, and about 0 mol % PEG lipid. In some aspects, the LNP of the disclosure comprises about 40 mol % ionizable lipid, about 10 mol % non-cationic helper lipid, about 50 mol % phytosterol and cholesterol, and about 0 mol % PEG lipid.

In some aspects, the LNP of the disclosure comprises about 45 mol % ionizable lipid, about 10 mol % non-cationic helper lipid, about 45 mol % phytosterol, and about 0 mol % PEG lipid. In some aspects, the LNP of the disclosure comprises about 45 mol % ionizable lipid, about 10 mol % non-cationic helper lipid, about 45 mol % phytosterol and a structural lipid, and about 0 mol % PEG lipid. In some aspects, the LNP of the disclosure comprises about 45 mol % ionizable lipid, about 10 mol % non-cationic helper lipid, about 45 mol % phytosterol and cholesterol, and about 0 mol % PEG lipid.

In some aspects, the LNP of the disclosure comprises about 50 mol % ionizable lipid, about 0 mol % non-cationic helper lipid, about 48.5 mol % phytosterol, and about 1.5 mol % PEG lipid. In some aspects, the LNP of the disclosure comprises about 50 mol % ionizable lipid, about 0 mol % non-cationic helper lipid, about 48.5 mol % phytosterol and a structural lipid, and about 1.5 mol % PEG lipid. In some aspects, the LNP of the disclosure comprises about 50 mol % ionizable lipid, about 0 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 10 mol % non-cationic helper lipid, about 40 mol % phytosterol, and about 0 mol % PEG lipid. In some aspects, the LNP of the disclosure comprises about 50 mol % ionizable lipid, about 10 mol % non-cationic helper lipid, about 40 mol % phytosterol and a structural lipid, and about 0 mol % PEG lipid. In some aspects, the LNP of the disclosure comprises about 50 mol % ionizable lipid, about 10 mol % non-cationic helper lipid, about 40 mol % phytosterol and cholesterol, and about 0 mol % PEG lipid.

In some aspects, the LNP of the disclosure comprises about 55 mol % ionizable lipid, about 10 mol % non-cationic helper lipid, about 35 mol % phytosterol, and about 0 mol % PEG lipid. In some aspects, the LNP of the disclosure comprises about 55 mol % ionizable lipid, about 10 mol % non-cationic helper lipid, about 35 mol % phytosterol and a structural lipid, and about 0 mol % PEG lipid. In some aspects, the LNP of the disclosure comprises about 55 mol % ionizable lipid, about 10 mol % non-cationic helper lipid, about 35 mol % phytosterol and cholesterol, and about 0 mol % PEG lipid.

In some aspects, the LNP of the disclosure comprises about 60 mol % ionizable lipid, about 10 mol % non-cationic helper lipid, about 30 mol % phytosterol, and about 0 mol % PEG lipid. In some aspects, the LNP of the disclosure comprises about 60 mol % ionizable lipid, about 10 mol % non-cationic helper lipid, about 30 mol % phytosterol and a structural lipid, and about 0 mol % PEG lipid. In some aspects, the LNP of the disclosure comprises about 60 mol % ionizable lipid, about 10 mol % non-cationic helper lipid, about 30 mol % phytosterol and cholesterol, and about 0 mol % PEG lipid.

In some aspects with respect to the embodiments herein, the phytosterol and a structural lipid components of a LNP of the disclosure comprises between about 10:1 and 1:10 phytosterol to structural lipid, such as about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9 and 1:10 phytosterol to structural lipid (e.g. beta-sitosterol to cholesterol).

In some embodiments, the phytosterol component of the LNP is a blend of the phytosterol and a structural lipid, such as cholesterol, wherein the phytosterol (e.g., beta-sitosterol) and the structural lipid (e.g., cholesterol) are each present at a particular mol %. For example, in some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17,18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

The amount of a nucleic acid molecule in a lipid nanoparticle may depend on the size, composition, desired target and/or application, or other properties of the lipid nanoparticle as well as on the properties of the therapeutic and/or prophylactic. For example, the amount of an RNA useful in a lipid nanoparticle may depend on the size, sequence, and other characteristics of the RNA. The relative amounts of one or more nucleic acid molecules and other elements (e.g., lipids) in a lipid nanoparticle may also vary. In some embodiments, the wt/wt ratio of the ionizable lipid component to one or more nucleic acid molecules, in a lipid nanoparticle may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the ionizable lipid component to one or more nucleic acid molecules may be from about 10:1 to about 40:1. In certain embodiments, the wt/wt ratio is about 20:1. The amount of one or more nucleic acid molecules in a LNP may, for example, be measured using absorption spectroscopy (e.g., ultraviolet-visible spectroscopy).

In some embodiments, a lipid nanoparticle includes one or more RNAs, and one or more ionizable lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 5.7:1, about 5.8:1, about 5.9:1, about 6.0:1, about 6.5:1, or about 7.0:1. For example, the N:P ratio may be about 5.67:1. In another embodiment, the N:P ratio may be about 5.8:1.

In some embodiments, the formulation including a lipid nanoparticle may further includes a salt, such as a chloride salt.

In some embodiments, the formulation including a lipid nanoparticle may further includes a sugar such as a disaccharide. In some embodiments, the formulation further includes a sugar but not a salt, such as a chloride salt.

### Physical Properties

The characteristics of a lipid nanoparticle may depend on the components thereof. For example, a lipid nanoparticle including cholesterol as a structural lipid may have different characteristics than a lipid nanoparticle that includes a different structural lipid. Similarly, the characteristics of a lipid nanoparticle may depend on the absolute or relative amounts of its components. For instance, a lipid nanoparticle including a higher molar fraction of a phospholipid may have different characteristics than a lipid nanoparticle including a lower molar fraction of a phospholipid. Characteristics may also vary depending on the method and conditions of preparation of the lipid nanoparticle.

Lipid nanoparticles may be characterized by a variety of methods. For example, microscopy (e.g., transmission electron microscopy or scanning electron microscopy) may be used to examine the morphology and size distribution of a lipid nanoparticle. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a lipid nanoparticle, such as particle size, polydispersity index, and zeta potential.

The mean size of a lipid nanoparticle may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a lipid nanoparticle may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a lipid nanoparticle may be from about 70 nm to about 100 nm. In a particular embodiment, the mean size may be about 80 nm. In other embodiments, the mean size may be about 100 nm.

A lipid nanoparticle may be relatively homogenous. A polydispersity index may be used to indicate the homogeneity of a LNP, e.g., the particle size distribution of the lipid nanoparticles. As used herein, the “polydispersity index” is a ratio that describes the homogeneity of the particle size distribution of a system. A small value, e.g., less than 0.3, indicates a narrow particle size distribution. A small (e.g., less than 0.3) polydispersity index generally indicates a narrow particle size distribution. A lipid nanoparticle may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a lipid nanoparticle may be from about 0.10 to about 0.20.

The zeta potential of a lipid nanoparticle may be used to indicate the electrokinetic potential of the composition. As used herein, the “zeta potential” is the electrokinetic potential of a lipid, e.g., in a particle composition.

For example, the zeta potential may describe the surface charge of a lipid nanoparticle. Lipid nanoparticles with relatively low charges, positive or negative, are generally desirable, as more highly charged species may interact undesirably with cells, tissues, and other elements in the body. In some embodiments, the zeta potential of a lipid nanoparticle may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a nucleic acid molecule describes the amount of nucleic acid molecule that is encapsulated or otherwise associated with a lipid nanoparticle after preparation, relative to the initial amount provided. The encapsulation efficiency is desirably high (e.g., close to 100%). The encapsulation efficiency may be measured, for example, by comparing the amount of nucleic acid molecule in a solution containing the lipid nanoparticle before and after breaking up the lipid nanoparticle with one or more organic solvents or detergents. Fluorescence may be used to measure the amount of free nucleic acid molecules (e.g., RNA) in a solution. For the lipid nanoparticles described herein, the encapsulation efficiency of a nucleic acid molecule may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A lipid nanoparticle may optionally comprise one or more coatings. For example, a lipid nanoparticle may be formulated in a capsule, film, or tablet having a coating. A capsule, film, or tablet including a composition described herein may have any useful size, tensile strength, hardness, or density.

### Pharmaceutical Compositions

The present disclosure includes pharmaceutical compositions comprising an mRNA or a nanoparticle (e.g., a lipid nanoparticle) described herein, in combination with one or more pharmaceutically acceptable excipient, carrier or diluent. In particular embodiments, the mRNA is present in a nanoparticle, e.g., a lipid nanoparticle. In particular embodiments, the mRNA or nanoparticle is present in a pharmaceutical composition. In various embodiments, the one or more mRNA present in the pharmaceutical composition is encapsulated in a nanoparticle, e.g., a lipid nanoparticle. In particular embodiments, the molar ratio of the first mRNA to the second mRNA is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In particular embodiments, the molar ratio of the first mRNA to the second mRNA is greater than 1:1.

In some embodiments, a first mRNA encoding OX40L, a second mRNA encoding tethered IL-12 and at least a third mRNA encoding cell-associated IL-15 are co-formulated (e.g., in an LNP) at varying weight ratios, for example, with equivalent amounts (by weight) of each mRNA or with any one of the mRNA present at 2, 3, 4, 5, 6, 7, 8, 9, 10, 11, 12, 13, 14, 15, 16, 17, 18, 19, 20, 25, 30, 35, 40, 45, 50, 75, or 100 times the amount (by weight) of the other mRNAs.

In some embodiments, a first mRNA encoding OX40L, a second mRNA encoding tethered IL-12 and at least a third mRNA encoding a cell-associated IL-15 are co-formulated (e.g., in an LNP) at varying mass quantity of each mRNA or with any one of the mRNAs present in the formulation. For example, a first mRNA is co-formulated relative to a second and/or third mRNA in a formulation (e.g., an LNP) in which the first mRNA is present in an amount from 10-100%, 20-80%, 30-70%, or 40-50% the mass quantity of the amount of the second mRNA and/or third mRNAs. In another embodiment, a first mRNA and a second mRNA are co-formulated relative to a third mRNA in a formulation (e.g., an LNP) in which the first mRNA and second mRNA are present in an amount from 10-100%, 20-80%, 30-70%, or 40-50% the mass quantity of the third mRNA.

In some embodiments, the OX40L:tethered IL-12:cell-associated IL-15 mRNAs are co-formulated (e.g., in an LNP) at a weight (mass) ratio such that the tethered IL-12 and cell-associated IL-15 mRNAs are at about equal amounts and the OX40L mRNA is present at a lower weight (mass) amount, such as 1.5, 2.0. 2.5, 3.0, 3.5, 4.0, 4.5 or 5.0 times less weight (mass) amount. In some embodiments, the OX40L:tethered IL-12:cell-associated IL-15 mRNAs are co-formulated (e.g., in an LNP) at a weight (mass) ratio of 1:1:1. In some embodiments, the OX40L:tethered IL-12:cell-associated IL-15 mRNAs are co-formulated (e.g., in an LNP) at a weight (mass) ratio of 0.1:1:1.

In some embodiments, any one of mRNAs encoding OX40L, tethered IL-12 and cell-associated IL-15 is co-formulated (e.g., in an LNP) with the other mRNAs from 10%-100% the mass quantity of the other mRNAs. In some embodiments, the OX40L:tethered IL-12:cell-associated IL-15 mRNAs are co-formulated (e.g., in an LNP) at a weight (mass) ratio of 0.1-1:0.1-1:0.1-1.

In some embodiments, the mRNA(s) encoding a cell-associated IL-15 polypeptide is an mRNA encoding IL-15 and an mRNA encoding IL-15Rα co-formulated (e.g., in an LNP) at a 1:1 molar ratio. In some embodiments, the mRNA(s) encoding a cell-associated IL-15 polypeptide is an mRNA encoding IL-15 and an mRNA encoding IL-15Rα co-formulated (e.g., in an LNP) at a 1:1.4 weight (mass) ratio. Accordingly, in some embodiments, the weight (mass) ratio of mRNAs encoding OX40L:tethered IL-12:IL-15:IL-15Rα is 2.4:2.4:1:1.4. In one embodiment, the disclosure provides an LNP comprising mRNAs encoding OX40L:tethered IL-12:IL-15:IL-15Rα at a weight (mass) ratio of 2.4:2.4:1:1.4.

In some embodiments, the weight (mass) ratio of a first mRNA encoding OX40L to a second mRNA encoding tethered IL-12 to a third mRNA encoding IL-15 operably linked to IL-15Rα is 1:1:1. In one embodiment, the disclosure provides an LNP comprising mRNAs encoding OX40L:tethered IL-12:IL-15:IL-15Rα at a weight (mass) ratio of 1:1:1. In some embodiments, the weight (mass) ratio of a first mRNA encoding OX40L to a second mRNA encoding tethered IL-12 to a third mRNA encoding IL-15 operably linked to IL-15Rα is about 0.1:1:1. In one embodiment, the disclosure provides an LNP comprising mRNAs encoding OX40L:tethered IL-12:IL-15:IL-15Rα at a weight (mass) ratio of 0.1:1:1. In some embodiments, the weight (mass) ratio of a first mRNA encoding OX40L to a second mRNA encoding tethered IL-12 to a third mRNA encoding IL-15 to a fourth mRNA encoding IL-15Rα is 2.4:2.4:1:1.4. In one embodiment, the disclosure provides an LNP comprising mRNAs encoding OX40L:tethered IL-12:IL-15:IL-15Rα at a weight (mass) ratio of 2.4:2.4:1:1.4. In some embodiments, the molar ratio of a first mRNA encoding OX40L to a second mRNA encoding tethered IL-12 to a third mRNA encoding IL-15 to a fourth mRNA encoding IL-15Rα is about 0.24:2.4:1:1.4. In one embodiment, the disclosure provides an LNP comprising mRNAs encoding OX40L:tethered IL-12:IL-15:IL-15Rα at a weight (mass) ratio of 0.24:2.4:1:1.4.

Pharmaceutical compositions may optionally include one or more additional active substances, for example, therapeutically and/or prophylactically active substances. Pharmaceutical compositions of the present disclosure may be sterile and/or pyrogen-free. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference in its entirety). In particular embodiments, a pharmaceutical composition comprises an mRNA and a lipid nanoparticle, or complexes thereof.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100%, e.g., between 0.5% and 70%, between 1% and 30%, between 5% and 80%, or at least 80% (w/w) active ingredient.

The mRNAs of the disclosure can be formulated using one or more excipients to: (1) increase stability; (2) increase cell transfection; (3) permit the sustained or delayed release (e.g., from a depot formulation of the mRNA); (4) alter the biodistribution (e.g., target the mRNA to specific tissues or cell types); (5) increase the translation of a polypeptide encoded by the mRNA in vivo; and/or (6) alter the release profile of a polypeptide encoded by the mRNA in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles (e.g., liposomes and micelles), polymers, lipoplexes, core-shell nanoparticles, peptides, proteins, carbohydrates, cells transfected with mRNAs (e.g., for transplantation into a subject), hyaluronidase, nanoparticle mimics and combinations thereof. Accordingly, the formulations of the disclosure can include one or more excipients, each in an amount that together increases the stability of the mRNA, increases cell transfection by the mRNA, increases the expression of a polypeptide encoded by the mRNA, and/or alters the release profile of an mRNA-encoded polypeptide. Further, the mRNAs of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Excipients may include, for example: antiadherents, antioxidants, binders, coatings, compression aids, disintegrants, dyes (colors), emollients, emulsifiers, fillers (diluents), film formers or coatings, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

In some embodiments, the formulations described herein may include at least one pharmaceutically acceptable salt. Examples of pharmaceutically acceptable salts that may be included in a formulation of the disclosure include, but are not limited to, acid addition salts, alkali or alkaline earth metal salts, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In some embodiments, the formulations described herein may contain at least one type of mRNA. As a non-limiting example, the formulations may contain 1, 2, 3, 4, 5 or more than 5 mRNAs described herein. In some embodiments, the formulations described herein may contain at least one mRNA encoding a polypeptide and at least one nucleic acid sequence such as, but not limited to, an siRNA, an shRNA, a snoRNA, and an miRNA.

Liquid dosage forms for e.g., parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and/or suspending agents. In certain embodiments for parenteral administration, compositions are mixed with solubilizing agents such as CREMAPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, pharmaceutical compositions including at least one mRNA described herein are administered to mammals (e.g., humans). Although the descriptions of pharmaceutical compositions provided herein are principally directed to pharmaceutical compositions which are suitable for administration to humans, it will be understood by the skilled artisan that such compositions are generally suitable for administration to any other animal, e.g., to a non-human mammal. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys. In particular embodiments, a subject is provided with two or more mRNAs described herein. In particular embodiments, the first and second mRNAs are provided to the subject at the same time or at different times, e.g., sequentially. In particular embodiments, the first and second mRNAs are provided to the subject in the same pharmaceutical composition or formulation, e.g., to facilitate uptake of both mRNAs by the same cells.

The present disclosure also includes kits comprising a container comprising a mRNA encoding a polypeptide that enhances an immune response. In another embodiment, the kit comprises a container comprising a mRNA encoding a polypeptide that enhances an immune response, as well as one or more additional mRNAs encoding one or more antigens or interest. In other embodiments, the kit comprises a first container comprising the mRNA encoding a polypeptide that enhances an immune response and a second container comprising one or more mRNAs encoding one or more antigens of interest. In particular embodiments, the mRNAs for enhancing an immune response and the mRNA(s) encoding an antigen(s) are present in the same or different nanoparticles and/or pharmaceutical compositions. In particular embodiments, the mRNAs are lyophilized, dried, or freeze-dried.

### Methods of Use

In some embodiments, the disclosure provides a method for treating a cancer in a subject in need thereof, e.g., a human subject. In some embodiments, the disclosure provides a method for enhancing an immune response to a cancer. In some embodiments, the disclosure provides a method for enhancing an immune response to a leukemic cell (e.g., an AML cell). In some embodiments, the disclosure provides a method for enhancing an immune response to a solid tumor. In some embodiments, enhancing an immune response comprises stimulating cytokine production. In another embodiment, enhancing an immune response comprises enhancing cellular immunity (T cell responses), such activating T cells. In some embodiments, enhancing an immune response comprises activating NK cells. Enhancement of an immune response in a subject can be evaluated by a variety of methods established in the art for assessing immune response, including but not limited to determining the level of T cell activation and NK cell activation by intracellular staining of activation markers.

### Disseminated Cancers

In some embodiments, the disclosure provides a method for treating a disseminated cancer in a subject in need thereof, e.g., a human subject. In some embodiments, treatment of a disseminated cancer comprises enhancing an immune response to the disseminated cancer. Disseminated cancers include metastatic cancers and cancers located within the circulation, e.g., the blood, of a subject which do not ordinarily form solid tumors. Disseminated cancers that do not ordinarily form solid tumors include, but are not limited to, cancers having significant myeloid populations, as well as multiple myeloma and B cell leukemias.

In some embodiments, the disseminated cancer is a hematological cancer. As used herein, the term “hematological cancer” includes a lymphoma, leukemia, myeloma or a lymphoid malignancy, as well as a cancer of the spleen and lymph nodes. Exemplary lymphomas include both B cell lymphomas (a B-cell hematological cancer) and T cell lymphomas. B-cell lymphomas include both Hodgkin's lymphomas and most non-Hodgkin's lymphomas. Non-limiting examples of B cell lymphomas include diffuse large B-cell lymphoma, follicular lymphoma, mucosa-associated lymphatic tissue lymphoma, small cell lymphocytic lymphoma (overlaps with chronic lymphocytic leukemia), mantle cell lymphoma (MCL), Burkitt's lymphoma, mediastinal large B cell lymphoma, Waldenstrom macroglobulinemia, nodal marginal zone B cell lymphoma, splenic marginal zone lymphoma, intravascular large B-cell lymphoma, primary effusion lymphoma, lymphomatoid granulomatosis. Non-limiting examples of T cell lymphomas include extranodal T cell lymphoma, cutaneous T cell lymphomas, anaplastic large cell lymphoma, and angioimmunoblastic T cell lymphoma. Hematological malignancies also include leukemia, such as, but not limited to, secondary leukemia, chronic lymphocytic leukemia, acute myelogenous leukemia, chronic myelogenous leukemia, and acute lymphoblastic leukemia. Hematological malignancies further include myelomas, such as, but not limited to, multiple myeloma and smoldering multiple myeloma. Other hematological and/or B cell- or T-cell-associated cancers are encompassed by the term hematological malignancy.

In some embodiments, the disseminated cancer is a myeloid malignancy Myeloid malignancies include myelodysplastic syndrome (MDS), myeloproliferative disorders or neoplasms (MPD) and acute myeloid leukemia (AML).

In some embodiments, the disseminated cancer is a metastases of a primary tumor. In some embodiments, the disseminated cancer is a metastases of a previous metastases of a primary tumor. In some embodiments, disseminated cancer cells are detached from a primary tumor or metastases and enter the circulation. Such disseminated cancer cells can form tumors in locations distal from the primary tumor or metastases from which the cells are derived.

### Solid Tumors

In some embodiments, the disclosure provides a method for treating a solid tumor in a subject in need thereof, e.g., a human subject. In some embodiments, treatment of a solid tumor comprises enhancing an immune response to the solid tumor.

In some embodiments, the method comprises intratumoral administration of the compositions and/or mRNAs disclosed herein. In some embodiments, intratumoral administration promotes an immune response systemically. In some embodiments, intratumoral administration results in the shrinking or delaying of untreated tumors by promotion of an immune response systemically.

A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Cancers and/or tumors amenable to treatment in accordance with the methods of the instant invention include those accessible via direct intratumoral and/or regional administration, i.e., administration in the region of a target tumor. For example, tumors accessible to administration with a simple syringe injection are readily amenable to treatment. Also amenable to treatment are tumors in which injection requires some imaging and/or guided administration, and/or those in which injection is possible via image-guided percutaneous injection, or catheter/cannula directly into site, or endoscopy.

In some embodiments, the solid tumor comprises a tumor microenvironment that is immunogenic. In some embodiments, immunogenic tumor microenvironments are characterized by greater T-cell infiltration and Th1 cytokine expression. In some embodiments, the solid tumors comprise a tumor microenvironment that is immunologically barren. In some embodiments, immunologically barren tumor microenvironments are characterized by sparse T-cell infiltrate. In some embodiments, the solid tumor is resistant and/or unresponsive to immune checkpoint therapy. Mosley et al. describe these various tumor microenvironments (Mosley et al. Rational Selection of Syngenic Preclinical Tumor Models for Immunotherapeutic Drug Discovery, Cancer Immunology Research, doi: 10.1158/2326-6066.CIR-16-0114 (2016), incorporated herein by this reference).

In certain embodiments, the mRNAs described herein can be used to modulate tumor microenvironments and/or can be selected for treatment based on the tumor microenvironment in the subject to be treated. In some embodiments, the mRNAs are used to treat a tumor that has an inflamed tumor microenvironment. In some embodiments, the mRNAs are used to treat a tumor that has an immunosuppressive tumor microenvironment. In some embodiments, the mRNAs are used to treat a tumor that has an immunologically barren tumor microenvironment.

In some embodiments, any of the methods described herein comprise administering to the subject a composition of the disclosure (or lipid nanoparticle thereof, or pharmaceutical composition thereof) comprising:

(i) an mRNA encoding a human OX40L polypeptide and an mRNA encoding a human IL-12 polypeptide operably linked to a membrane domain comprising a transmembrane domain;

(ii) an mRNA encoding a human OX40L polypeptide, an mRNA encoding a human IL-15 polypeptide and an mRNA encoding a human IL-15Rαpolypeptide;

(iii) an mRNA encoding a human OX40L polypeptide and an mRNA encoding a human IL-15 polypeptide operably linked to a human IL-15Rα polypeptide;

(iv) an mRNA encoding a human IL-12 polypeptide operably linked to a membrane domain comprising a transmembrane domain, an mRNA encoding a human IL-15 polypeptide and an mRNA encoding a human IL-15Rα polypeptide;

(v) an mRNA encoding a human IL-12 polypeptide operably linked to a membrane domain comprising a transmembrane domain and an mRNA encoding a human IL-15 polypeptide operably linked to a human IL-15Rα polypeptide;

(vi) an mRNA encoding a human OX40L polypeptide, an mRNA encoding a human IL-12 polypeptide operably linked to a membrane domain comprising a transmembrane domain, an mRNA encoding a human IL-15 polypeptide and an mRNA encoding a human IL-15Rα polypeptide; or

(vii) an mRNA encoding a human OX40L polypeptide, an mRNA encoding a human IL-12 polypeptide operably linked to a membrane domain comprising a transmembrane domain and an mRNA encoding a human IL-15 polypeptide operably linked to a human IL-15Rα polypeptide.

When multiple mRNAs are used, they can be coformulated, e.g., in the same lipid nanoparticles, and/or can be co-administered. Alternatively, different mRNAs can be administered to the subject at different times. For example, one mRNA composition (e.g., encoding an OX40L polypeptide) can be administered 1-30 days, e.g., 3 days, 5 days, 7 days, 10 days, 14 days, 21 days, 28 days, prior to administering a second mRNA composition (e.g., encoding an IL-12 polypeptide and/or an IL-15 polypeptide).

Compositions of the disclosure are administered to the subject at an effective amount. In general, an effective amount of the composition will allow for efficient production of the encoded polypeptide in the cell. Metrics for efficiency may include polypeptide translation (indicated by polypeptide expression), level of mRNA degradation, and immune response indicators.

The methods of the disclosure for treating a cancer (e.g., solid tumor or disseminated cancer such as a myeloid malignancy) can be used in a variety of clinical or therapeutic applications. For example, the methods can be used to stimulate anti-cancer immunity in a subject with a cancer (e.g., anti-malignancy immunity in a subject with a myeloid malignancy).

In certain embodiments, a subject is administered at least one mRNA composition described herein. In related embodiments, the subject is provided with or administered a nanoparticle (e.g., a lipid nanoparticle) comprising the mRNA(s). In further related embodiments, the subject is provided with or administered a pharmaceutical composition of the disclosure to the subject. In particular embodiments, the pharmaceutical composition comprises an mRNA(s) as described herein, or it comprises a nanoparticle comprising the mRNA(s). In particular embodiments, the mRNA(s) is present in a nanoparticle, e.g., a lipid nanoparticle. In particular embodiments, the mRNA(s) or nanoparticle is present in a pharmaceutical composition.

In some embodiments, the mRNA(s), nanoparticle, or pharmaceutical composition is administered to the patient parenterally. In particular embodiments, the subject is a mammal, e.g., a human. In various embodiments, the subject is provided with an effective amount of the mRNA(s).

The methods of treating cancer can further include treatment of the subject with additional agents that enhance an anti-tumor response in the subject and/or that are cytotoxic to the tumor (e.g., chemotherapeutic agents). Suitable therapeutic agents for use in combination therapy include small molecule chemotherapeutic agents, including protein tyrosine kinase inhibitors, as well as biological anti-cancer agents, such as anti-cancer antibodies, including but not limited to those discussed further below. Combination therapy can include administering to the subject an immune checkpoint inhibitor to enhance anti-tumor immunity, such as PD-1 inhibitors, PD-L¹ inhibitors and CTLA-4 inhibitors, and combinations thereof (e.g., a PD-1 inhibitor+a CTLA-4 inhibitor, a PD-L¹ inhibitor+a CTLA-4 inhibitor or a PD-1 inhibitor+a PD-L¹ inhibitor). In one embodiment, an agent that modulates an immune checkpoint is an antibody. In another embodiment, an agent that modulates an immune checkpoint is a protein or small molecule modulator. In another embodiment, the agent (such as an mRNA) encodes an antibody modulator of an immune checkpoint. Non-limiting examples of immune checkpoint inhibitors that can be used in combination therapy include pembrolizumab, alemtuzumab, nivolumab, pidilizumab, ofatumumab, MEDI0680 and PDR001, AMP-224, PF-06801591, BGB-A317, REGN2810, SHR-1210, TSR-042, affimer, avelumab (MSB0010718C), atezolizumab (MPDL3280A), durvalumab (MEDI4736), BMS936559, ipilimumab, tremelimumab, AGEN1884, MEDI6469 and MOXR0916.

In one embodiment, a single dose of the mRNA(s) of the disclosure (e.g., an mRNA encoding a human OX40L polypeptide+an mRNA encoding a tethered human IL-12 polypeptide+an mRNA encoding a human IL-15 polypeptide+an mRNA encoding a human IL-15Rα polypeptide (or an mRNA encoding a human IL-15 polypeptide operably linked to a human IL-15Rα polypeptide)) is used in combination with treatment with at least one immune checkpoint inhibitor (e.g., anti-CTLA-4, anti-PD-L¹, anti-PD-1 or combinations thereof). In another embodiment, multiple doses (e.g., Q7D×3) of the mRNA(s) of the disclosure (e.g., an mRNA encoding a human OX40L polypeptide+an mRNA encoding a tethered human IL-12 polypeptide+an mRNA encoding a human IL-15 polypeptide+an mRNA encoding a human IL-15Rα polypeptide (or an mRNA encoding a human IL-15 polypeptide operably linked to a human IL-15Rα polypeptide)) are used in combination with treatment with at least one immune checkpoint inhibitor (e.g., anti-CTLA-4, anti-PD-L¹, anti-PD-1 or combinations thereof). Treatment with the immune checkpoint inhibitor(s) can comprise administration of a single dose of the checkpoint inhibitor(s) or, more typically, administration of multiple doses of the checkpoint inhibitors(s).

A pharmaceutical composition including one or more mRNAs of the disclosure may be administered to a subject by any suitable route. In some embodiments, compositions of the disclosure are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. In some embodiments, a composition is administered intramuscularly. However, the present disclosure encompasses the delivery of compositions of the disclosure by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In certain embodiments, compositions of the disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In particular embodiments, a dose of about 0.005 mg/kg to about 5 mg/kg of mRNA or nanoparticle of the disclosure may be administrated.

A dose may be administered one or more times per day, in the same or a different amount, to obtain a desired level of mRNA expression and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage is delivered using multiple administrations of a single dose (e.g., two, three, four, five, six, seven, eight, nine, ten, eleven, twelve, thirteen, fourteen, or more administrations), referred to as “fractionated dosing”. For example, a desired dosage of 2 mg/kg per week can be administered to a subject over the course of the week by administering 0.67 mg/kg three times a week instead of a single bolus dose of 2 mg/kg. In some embodiments, the fractionated dosing regimen results in enhanced anti-cancer efficacy relative to a single bolus of the same total dose. In some embodiments, the fractionated dosing regimen results in less toxicity relative to a single bolus of the same total dose. In some embodiments, a fractionated dosing regimen is better tolerated by a subject relative to a single bolus dose. In some embodiments, the enhanced efficacy of fractionated dosing is due to greater or enhanced exposure to the mRNA encoded polypeptides. Methods for measuring exposure include, but are not limited to, determining the concentration of the mRNA encoded polypeptides in a sample, determining the half-life of the mRNA encoded polypeptides, and/or determining the area under the curve (AUC) of drug concentration in a sample (e.g., blood plasma) versus time.

In some embodiments, a single dose may be administered, for example, prior to or after a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

In some embodiments, a pharmaceutical composition of the disclosure may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure, or imaging, diagnostic, or prophylactic compositions thereof in combination with agents that improve their bioavailability, reduce and/or modify their metabolism, inhibit their excretion, and/or modify their distribution within the body.

Exemplary therapeutic agents that may be administered in combination with the compositions of the disclosure include, but are not limited to, cytotoxic, chemotherapeutic, hypomethylating agents, pro-apoptotic agents, small molecules/kinase inhibitors, and other therapeutic agents including therapeutics approved for cancer, such as AML or MDS, now or at a later date. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

### Kits

In some embodiments, the disclosure provides a kit comprising the mRNAs described herein. For example, in some embodiments the comprises (i) an mRNA encoding an OX40L polypeptide; (ii) an mRNA encoding an IL-12 polypeptide; (iii) an mRNA encoding an IL-15 polypeptide; and (iv) an mRNA encoding an IL-15Rα polypeptide, co-formulated in a lipid nanoparticle. in some embodiments the comprises (i) an mRNA encoding an OX40L polypeptide; (ii) an mRNA encoding an IL-12 polypeptide; (iii) an mRNA encoding an IL-15 operably linked to an IL-15Rα polypeptide, co-formulated in a lipid nanoparticle. Accordingly, in some embodiments, a kit comprises a container comprising a lipid nanoparticle encapsulating the mRNAs described herein, and an optional pharmaceutically acceptable carrier, or a pharmaceutical composition, and a package insert comprising instructions for administration of the lipid nanoparticle or pharmaceutical composition. In some embodiments, a kit comprises a container comprising a lipid nanoparticle encapsulating the mRNAs described herein, and an optional pharmaceutically acceptable carrier, or a pharmaceutical composition, and a package insert comprising instructions for administration of the lipid nanoparticle or pharmaceutical composition for treating or delaying progression of a cancer (e.g., solid tumor or disseminated cancer such as a myeloid malignancy) in an individual. In some aspects, the package insert further comprises instructions for administration of the lipid nanoparticle or pharmaceutical composition in combination with a composition comprising a checkpoint inhibitor polypeptide and an optional pharmaceutically acceptable carrier for treating or delaying progression of a cancer (e.g., solid tumor or disseminated cancer such as a myeloid malignancy) in an individual.

In some embodiments, a kit comprises a medicament comprising a lipid nanoparticle encapsulating the mRNAs described herein, and an optional pharmaceutically acceptable carrier, or a pharmaceutical composition, and a package insert comprising instructions for administration of the medicament alone or in combination with a composition comprising a checkpoint inhibitor polypeptide and an optional pharmaceutically acceptable carrier. In some embodiments, a kit comprises a medicament comprising a lipid nanoparticle encapsulating the mRNAs described herein, and an optional pharmaceutically acceptable carrier, or a pharmaceutical composition, and a package insert comprising instructions for administration of the medicament alone or in combination with a composition comprising a checkpoint inhibitor polypeptide and an optional pharmaceutically acceptable carrier for treating or delaying progression of a cancer (e.g., solid tumor or disseminated cancer such as a myeloid malignancy) in an individual. In some aspects, the kit further comprises a package insert comprising instructions for administration of the first medicament prior to, current with, or subsequent to administration of the second medicament for treating or delaying progression of a cancer (e.g., solid tumor or disseminated cancer such as a myeloid malignancy) in an individual.

### Definitions

Abscopal effect: As used herein, “abscopal effect” refers to a phenomenon in the treatment of cancer, including metastatic cancer, where localized administration of a treatment (e.g., mRNAs encoding OX40L, tethered IL-12 and cell-associated IL-15) to a tumor causes not only a reduction in size of the treated tumor but also a reduction in size of tumors outside the treated area. In some embodiments, the abscopal effect is a local, regional abscopal effect, wherein a proximal or nearby tumor relative to the treated tumor is affected. In some embodiments, the abscopal effect occurs in a distal tumor relative to the treated tumor. In some embodiments, treatment (e.g., mRNAs encoding OX40L, tethered IL-12 and cell-associated IL-15) is administered via intratumoral injection, resulting in a reduction in tumor size of the injected tumor and a proximal or distal uninjected tumor.

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Cancer: As used herein, “cancer” is a condition involving abnormal and/or unregulated cell growth. The term cancer encompasses benign and malignant cancers. Exemplary non-limiting cancers include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In some embodiments, the cancer is liver cancer (e.g., hepatocellular carcinoma), ovarian cancer or colorectal cancer. In other embodiments, the cancer is a blood-based cancer or a hematopoietic cancer. In some embodiments, the cancer is a myeloid malignancy, such as AML.

Cell-Associated: As used herein, the term “cell-associated” refers to the location of an mRNA encoded polypeptide on the surface of a cell, either naturally (e.g., in a wild-type form) or by design due to alteration of the mRNA encoded polypeptide (e.g., via recombinant techniques) such that when expressed the polypeptide is associated with the cell surface. In some embodiments, “cell-associated” refers to an mRNA encoded polypeptide that is naturally associated with a cell surface (e.g., includes a transmembrane domain) or a combination of mRNA(s) that when expressed encode polypeptides that associate (e.g., form a complex) which is bound to a cell surface. For example, an mRNA encoding IL-15 and an mRNA encoding IL-15Rα when expressed form a complex in which IL-15 associates with the membrane bound receptor, thereby confining the IL-15 to the surface of a cell when bound to the receptor. In other embodiments, “cell-associated” refers to an mRNA encoding a naturally soluble polypeptide (e.g., a cytokine, such as IL-12) which is engineered to comprise a membrane domain (e.g., a transmembrane domain), that confines the polypeptide to the surface of a cell. This is also referred to herein as a tethered polypeptide or tethered cytokine.

Cleavable Linker: As used herein, the term “cleavable linker” refers to a linker, typically a peptide linker (e.g., about 5-30 amino acids in length, typically about 10-20 amino acids in length) that can be incorporated into multicistronic mRNA constructs such that equimolar levels of multiple genes can be produced from the same mRNA. Non-limiting examples of cleavable linkers include the 2A family of peptides, including F2A, P2A, T2A and E2A, first discovered in picornaviruses, that when incorporated into an mRNA construct (e.g., between two polypeptide domains) function by making the ribosome skip the synthesis of a peptide bond at C-terminus of the 2A element, thereby leading to separation between the end of the 2A sequence and the next peptide downstream.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., an isolated mRNA, nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle or an isolated mRNA) and a cell may be contacted, for example, by adding the composition to the culture medium of the cell and may involve or result in transfection. Moreover, more than one cell may be contacted by a nanoparticle composition.

Disseminated cancer: As used herein the term “disseminated cancer” refers to circulating cancer cells within a subject. In some embodiments, disseminated cancer cells have detached from a primary tumor or metastases. In some embodiments, disseminated cancers include those that do not ordinarily form solid tumors and are found throughout the circulation of a subject, e.g., in the blood of a subject. In some embodiments, disseminated cancer cells are those derived from the hematopoietic lineage. In some embodiments, disseminated cancers include those having significant myeloid populations such as myeloid malignancies, along with lymphomas, leukemias etc.

Encapsulate: As used herein, the term “encapsulate” means to enclose, surround, or encase. In some embodiments, a compound, an mRNA, or other composition may be fully encapsulated, partially encapsulated, or substantially encapsulated. For example, in some embodiments, an mRNA of the disclosure may be encapsulated in a lipid nanoparticle, e.g., a liposome.

Effective amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent. In some embodiments, a therapeutically effective amount is an amount of an agent to be delivered (e.g., nucleic acid, drug, therapeutic agent, diagnostic agent or prophylactic agent) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Fractionated dosing: As used herein, “fractionated dosing” refers to a dosing regimen that involves taking an intended dose of mRNA (e.g., total amount of mRNA) and dividing it into at least two doses over a specified period of time (dosing interval, e.g., weekly, biweekly, bimonthly) such that the intended dose or total amount of mRNA is administered to a subject in multiple doses over the period of time rather than a single bolus dose of the intended dose. In some embodiments, a dose is fractionated into two, three, four, five, six, seven, eight, nine or ten doses. In some embodiments, fractionated dosing includes an infusion in which the dose is provided constantly over time.

Fragment: A “fragment,” as used herein, refers to a portion. For example, fragments of proteins may include polypeptides obtained by digesting full-length protein isolated from cultured cells or obtained through recombinant DNA techniques.

Heterologous: As used herein, “heterologous” indicates that a sequence (e.g., an amino acid sequence or the nucleic acid that encodes an amino acid sequence) is not normally present in a given polypeptide or nucleic acid. For example, an amino acid sequence that corresponds to a domain or motif of one protein may be heterologous to a second protein.

Hydrophobic amino acid: As used herein, a “hydrophobic amino acid” is an amino acid having an uncharged, nonpolar side chain. Examples of naturally occurring hydrophobic amino acids are alanine (Ala), valine (Val), leucine (Leu), isoleucine (Ile), proline (Pro), phenylalanine (Phe), methionine (Met), and tryptophan (Trp).

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two mRNA sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux et al., Nucleic Acids Research, 12(1): 387,1984, BLASTP, BLASTN, and FASTA, Altschul, S. F. et al., J. Molec. Biol., 215, 403, 1990.

Immune checkpoint inhibitor: An “immune checkpoint inhibitor” or simply “checkpoint inhibitor” refers to a molecule that prevents immune cells from being turned off by cancer cells. As used herein, the term checkpoint inhibitor refers to polypeptides (e.g., antibodies) or polynucleotides encoding such polypeptides (e.g., mRNAs) that neutralize or inhibit inhibitory checkpoint molecules such as cytotoxic T-lymphocyte-associated protein 4 (CTLA-4), programmed death 1 receptor (PD-1), or PD-1 ligand 1 (PD-L¹).

Immune response: The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues. In some cases, the administration of a nanoparticle comprising a lipid component and an encapsulated therapeutic agent can trigger an immune response, which can be caused by (i) the encapsulated therapeutic agent (e.g., an mRNA), (ii) the expression product of such encapsulated therapeutic agent (e.g., a polypeptide encoded by the mRNA), (iii) the lipid component of the nanoparticle, or (iv) a combination thereof.

Insertion: As used herein, an “insertion” or an “addition” refers to a change in an amino acid or nucleotide sequence resulting in the addition of one or more amino acid residues or nucleotides, respectively, to a molecule as compared to a reference sequence, for example, the sequence found in a naturally-occurring molecule. For example, an amino acid sequence of a heterologous polypeptide (e.g., a BH3 domain) may be inserted into a scaffold polypeptide (e.g. a SteA scaffold polypeptide) at a site that is amenable to insertion. In some embodiments, an insertion may be a replacement, for example, if an amino acid sequence that forms a loop of a scaffold polypeptide (e.g., loop 1 or loop 2 of SteA or a SteA derivative) is replaced by an amino acid sequence of a heterologous polypeptide.

Insertion Site: As used herein, an “insertion site” is a position or region of a scaffold polypeptide that is amenable to insertion of an amino acid sequence of a heterologous polypeptide. It is to be understood that an insertion site also may refer to the position or region of the mRNA that encodes the polypeptide (e.g., a codon of an mRNA that codes for a given amino acid in the scaffold polypeptide). In some embodiments, insertion of an amino acid sequence of a heterologous polypeptide into a scaffold polypeptide has little to no effect on the stability (e.g., conformational stability), expression level, or overall secondary structure of the scaffold polypeptide.

Intracellular domain: As used herein, the terms “intracellular domain”, “IC” and “ICD” refer to the region of a polypeptide located inside a cell. In some embodiments, an intracellular domain transmits a signal to the cell. In some embodiments, the tethered IL-12 polypeptides encoded by the polynucleotides (e.g., mRNA) described herein, comprise an intracellular domain that transmits a signal to the cell. In some embodiments, the tethered IL-12 polypeptides encoded by the polynucleotides (e.g., mRNA) described herein, comprise an intracellular domain that does not transmit a signal to the cell.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Linker: As used herein, a “linker” (including a membrane linker, a subunit linker, and a heterologous polypeptide linker as referred to herein) refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof., Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Metastasis: As used herein, the term “metastasis” means the process by which cancer spreads from the place at which it first arose as a primary tumor to distant locations in the body. A secondary tumor that arose as a result of this process may be referred to as “a metastasis.”

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

microRNA (miRNA): As used herein, a “microRNA (miRNA)” is a small non-coding RNA molecule which may function in post-transcriptional regulation of gene expression (e.g., by RNA silencing, such as by cleavage of the mRNA, destabilization of the mRNA by shortening its polyA tail, and/or by interfering with the efficiency of translation of the mRNA into a polypeptide by a ribosome). A mature miRNA is typically about 22 nucleotides long.

microRNA-122 (miR-122): As used herein, “microRNA-122 (miR-122)” refers to any native miR-122 from any vertebrate source, including, for example, humans, unless otherwise indicated. miR-122 is typically highly expressed in the liver, where it may regulate fatty-acid metabolism. miR-122 levels are reduced in liver cancer, for example, hepatocellular carcinoma. miR-122 is one of the most highly-expressed miRNAs in the liver, where it regulates targets including but not limited to CAT-1, CD320, AldoA, Hjv, Hfe, ADAM10, IGFR1, CCNG1, and ADAM17. Mature human miR-122 may have a sequence of AACGCCAUUAUCACACUAAAUA (SEQ ID NO: 73, corresponding to hsa-miR-122-3p) or UGGAGUGUGACAAUGGUGUUUG (SEQ ID NO: 82, corresponding to hsa-miR-122-5p).

microRNA-21 (miR-21): As used herein, “microRNA-21 (miR-21)” refers to any native miR-21 from any vertebrate source, including, for example, humans, unless otherwise indicated. miR-21 levels are increased in liver cancer, for example, hepatocellular carcinoma, as compared to normal liver. Mature human miR-21 may have a sequence of UAGCUUAUCAGACUGAUGUUGA (SEQ ID NO: 84, corresponding to has-miR-21-5p) or 5′-CAACACCAGUCGAUGGGCUGU-3′ (SEQ ID NO: 85, corresponding to has-miR-21-3p).

microRNA-142 (miR-142): As used herein, “microRNA-142 (miR-142)” refers to any native miR-142 from any vertebrate source, including, for example, humans, unless otherwise indicated. miR-142 is typically highly expressed in myeloid cells. Mature human miR-142 may have a sequence of UGUAGUGUUUCCUACUUUAUGGA (SEQ ID NO: 127, corresponding to hsa-miR-142-3p) or CAUAAAGUAGAAAGCACUACU (SEQ ID NO: 128, corresponding to hsa-miR-142-5p).

microRNA (miRNA) binding site: As used herein, a “microRNA (miRNA) binding site” refers to a miRNA target site or a miRNA recognition site, or any nucleotide sequence to which a miRNA binds or associates. In some embodiments, a miRNA binding site represents a nucleotide location or region of an mRNA to which at least the “seed” region of a miRNA binds. It should be understood that “binding” may follow traditional Watson-Crick hybridization rules or may reflect any stable association of the miRNA with the target sequence at or adjacent to the microRNA site.

miRNA seed: As used herein, a “seed” region of a miRNA refers to a sequence in the region of positions 2-8 of a mature miRNA, which typically has perfect Watson-Crick complementarity to the miRNA binding site. A miRNA seed may include positions 2-8 or 2-7 of a mature miRNA. In some embodiments, a miRNA seed may comprise 7 nucleotides (e.g., nucleotides 2-8 of a mature miRNA), wherein the seed-complementary site in the corresponding miRNA binding site is flanked by an adenine (A) opposed to miRNA position 1. In some embodiments, a miRNA seed may comprise 6 nucleotides (e.g., nucleotides 2-7 of a mature miRNA), wherein the seed-complementary site in the corresponding miRNA binding site is flanked by an adenine (A) opposed to miRNA position 1. When referring to a miRNA binding site, an miRNA seed sequence is to be understood as having complementarity (e.g., partial, substantial, or complete complementarity) with the seed sequence of the miRNA that binds to the miRNA binding site.

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Myeloid Malignancy: As used herein “myeloid malignancy” refers to both chronic and acute clonal disorders that are characterized by acquired somatic mutation(s) in hematopoietic progenitor cells, such as myelodysplastic disorders (MDS) and myeloproliferative neoplasms (MPN). Exemplary myeloid malignancies include, but are not limited to, acute myeloid leukemia (AML) and chronic meylomonocytic leukemia (CMML). Further, MPNs comprise a variety of disorders, such as chronic myeloid leukemia (CML) and non-CML MPNs such as polycythemia vera (PV), essential thrombocythemia (ET) and primary myelofibrosis (PMF).

Nanoparticle: As used herein, “nanoparticle” refers to a particle having any one structural feature on a scale of less than about 1000 nm that exhibits novel properties as compared to a hulk sample of the same material. Routinely, nanoparticles have any one structural feature on a scale of less than about 500 nm, less than about 200 nm, or about 100 nm. Also routinely, nanoparticles have any one structural feature on a scale of front about 50 nm to about 500 nm, from about 50 nm to about 200 nm or from about 70 to about 120 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Operably linked: As used herein, the phrase “operably linked” refers to a functional connection between two or more molecules, constructs, transcripts, entities, moieties or the like.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as an mRNA) into a cell.

Transmembrane domain: As used herein, the terms “transmembrane domain”, “TM” and “TMD” refer to the region of a polypeptide which crosses the plasma membrane of a cell.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Tumor Microenvironment”: As used herein, “tumor microenvironment” refers to the cellular compositions within a tumor with respect to the presence or absence of infiltrating immune and/or inflammatory cells, as well as the type(s) of such cells within the tumor. In some embodiments, a tumor microenvironment is an “inflamed tumor microenvironment”, which refers to the presence of immune and/or inflammatory cells infiltrated into the tumor, with the predominant cell type being granulocytes. In some embodiments, a tumor microenvironment is an “immunosuppressive tumor microenvironment”, which refers to the presence of immune and/or inflammatory cells infiltrated into the tumor, with the predominant cell types being monocytic cells and macrophages. In some embodiments, a tumor microenvironment is an “immunologically barren tumor microenvironment”, which refers to an absence of significant infiltration into the tumor of immune and/or inflammatory cells.

Type I integral membrane protein: As used herein, the term “type I integral membrane protein” refers to an integral membrane protein (i.e., proteins having at least one transmembrane domain that crosses the lipid bilayer) with its amino-terminus in the extracellular space and comprising one alpha-helical transmembrane domain.

Preventing: As used herein, the term “preventing” refers to partially or completely inhibiting the onset of one or more symptoms or features of cancer, including preventing a relapse or recurrence after successful treatment.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### OTHER EMBODIMENTS

The disclosure relates to the following embodiments. Throughout this section, the term embodiment is abbreviated as ‘E’ followed by an ordinal. For example, E1 is equivalent to Embodiment 1.
E1. A method of treating a myeloid malignancy in a subject in need thereof, the method comprising administering to the subject at least two messenger RNAs (mRNAs) selected from the group consisting of:

(i) an mRNA encoding an OX40L polypeptide;

(ii) an mRNA encoding an IL-15 polypeptide operably linked to an IL-15Rα polypeptide;

(iii) an mRNA encoding an IL-15 polypeptide and an mRNA encoding an IL-15Rα polypeptide; and

(iv) an mRNA encoding an IL-12 polypeptide operably linked to a membrane domain comprising a transmembrane domain.
E2. The method of embodiment 1, wherein the at least two mRNAs are selected from the group consisting of:

(i) an mRNA encoding an OX40L polypeptide and an mRNA encoding an IL-12 polypeptide operably linked to a membrane domain comprising a transmembrane domain;

(ii) an mRNA encoding an OX40L polypeptide, an mRNA encoding an IL-15 polypeptide and an mRNA encoding an IL-15Rα polypeptide;

(iii) an mRNA encoding an OX40L polypeptide and an mRNA encoding an IL-15 polypeptide operably linked to an IL-15Rα polypeptide;

(iv) an mRNA encoding an IL-15 polypeptide, an mRNA encoding an IL-15Rα polypeptide, and an mRNA encoding an IL-12 polypeptide operably linked to a membrane domain comprising a transmembrane domain;

(v) an mRNA encoding an IL-15 polypeptide operably linked to an IL-15Rα polypeptide, and an mRNA encoding an IL-12 polypeptide operably linked to a membrane domain comprising a transmembrane domain;

(vi) an mRNA encoding an OX40L polypeptide, an mRNA encoding an IL-15 polypeptide, an mRNA encoding an IL-15Rα polypeptide and an mRNA encoding an IL-12 polypeptide operably linked to a membrane domain comprising a transmembrane domain; and

(vii) an mRNA encoding an OX40L polypeptide, an mRNA encoding an IL-15 polypeptide operably linked to an IL-15Rα polypeptide, and an mRNA encoding an IL-12 polypeptide operably linked to a membrane domain comprising a transmembrane domain.
E3. The method of embodiment 1 or embodiment 2, wherein the OX40L polypeptide comprises the amino acid sequence set forth in SEQ ID NO: 1, or an amino acid sequence having at least 90% identity to the amino acid sequence set forth in SEQ ID NO: 1.
E4. The method of embodiment 3, wherein the OX40L polypeptide is encoded by a nucleotide sequence comprising the nucleotide sequence set forth SEQ ID NO: 11, or a nucleotide sequence having at least 80% identity to the nucleotide sequence set forth SEQ ID NO: 11.
E5. The method of any one of embodiments 1-4, wherein the IL-12 polypeptide comprises an IL-12 p40 subunit (IL-12B) polypeptide operably linked to an IL-12 p35 subunit (IL-12A) polypeptide.
E6. The method of embodiment 5, wherein the IL-12 polypeptide comprises the amino acid sequence set forth in SEQ ID NO: 39 or SEQ ID NO: 40, or an amino acid sequence having at least 90% identity to the amino acid sequence set forth in SEQ ID NO: 39 or SEQ ID NO: 40.
E7. The method of embodiment 6, wherein the IL-12 polypeptide is encoded by a nucleotide sequence comprising the nucleotide sequence set forth SEQ ID NO: 46, or a nucleotide sequence having at least 80% identity to the nucleotide sequence set forth SEQ ID NO: 46.
E8. The method of embodiment 5, wherein the IL-12B polypeptide is operably linked to the IL-12A polypeptide by a peptide linker.
E9. The method of embodiment 8, wherein the IL-12B polypeptide is located at the 5′ terminus of the IL-12A polypeptide, or the 5′ terminus of the peptide linker; or wherein the IL-12A polypeptide is located at the 5′ terminus of the IL-12B polypeptide, or the 5′ terminus of the peptide linker.
E10. The method of any one of embodiments 1-9, wherein the IL-12 polypeptide is operably linked to the membrane domain via a peptide linker.
E11. The method of anyone of embodiments 1-10, wherein the transmembrane domain of the membrane domain operably linked to the IL-12 polypeptide comprises a transmembrane domain derived from a Type I integral membrane protein.
E12. The method of anyone of embodiments 1-10, wherein the transmembrane domain of the membrane domain operably linked to the IL-12 polypeptide is selected from the group consisting of: a Cluster of Differentiation 8 (CD8) transmembrane domain, a Platelet-Derived Growth Factor Receptor (PDGFR) transmembrane domain, and a Cluster of Differentiation 80 (CD80) transmembrane domain.
E13. The method of embodiment 12, the PDGFR-beta transmembrane domain comprises the amino acid sequence set forth in SEQ ID NO: 42 the CD8 transmembrane domain comprises the amino acid sequence set forth in SEQ ID NO: 41; and the CD80 transmembrane domain comprises the amino acid sequence set forth in SEQ ID NO: 43.
E14. The method of any one of embodiments 1-13, wherein the membrane domain operably linked to the IL-12 polypeptide comprises an intracellular domain.
E15. The method of embodiment 14, wherein the intracellular domain is derived from the same polypeptide as the transmembrane domain, or wherein the intracellular domain is derived from a different polypeptide than the transmembrane domain is derived from.
E16. The method of embodiment 14, wherein the intracellular domain is selected from the group consisting of: a PDGFR intracellular domain, a truncated PDGFR intracellular domain, and a CD80 intracellular domain.
E17. The method of embodiment 16, wherein the intracellular domain is a PDGFR intracellular domain comprising a PDGFR-beta intracellular domain comprising the amino acid sequence set forth in SEQ ID NO: 48.
E18. The method of embodiment 16, wherein the intracellular domain is a truncated PDGFR intracellular domain comprising a PDGFR-beta intracellular domain truncated at E570 or G739.
E19. The method of embodiment 18, wherein the truncated PDGFR-beta intracellular domain truncated at E570 comprises the amino acid sequence set forth in SEQ ID NO: 49, and wherein the truncated PDGFR-beta transmembrane truncated at G739 comprises the amino acid sequence set forth in SEQ ID NO: 50.
E20. The method of embodiment 16, wherein the intracellular domain is a CD80 intracellular domain comprising the amino acid sequence set forth in SEQ ID NO: 47.
E21. The method of any one of embodiments 1-20, wherein the membrane domain operably linked to the IL-12 polypeptide comprises:

(i) a PDGFR-beta transmembrane domain and a PDGFR-beta intracellular domain;

(ii) a PDGFR-beta transmembrane domain and a truncated PDGFR-beta intracellular domain truncated at E570;

(iii) a PDGFR-beta transmembrane domain and a truncated PDGFR-beta intracellular domain truncated at G739; or

(iv) a CD80 transmembrane domain and a CD80 intracellular domain.
E22. The method of any one of embodiments 1-21, wherein the membrane domain is operably linked to the IL-12A polypeptide by a peptide linker, or wherein the membrane domain is operably linked to the IL-12B polypeptide by a peptide linker.
E23. The method of any one of embodiments 1-22, wherein the IL-15Rα polypeptide comprises a sushi domain.
E24. The method of embodiment 23, wherein the IL-15Rα polypeptide further comprises an intracellular domain and a transmembrane domain.
E25. The method of embodiment 24, wherein the intracellular domain and the transmembrane domain are derived from IL-15Rα.
E26. The method of embodiment 24, wherein the intracellular domain and the transmembrane domain are derived from a heterologous polypeptide.
E27. The method of any one of embodiments 1-26, wherein the IL-15 polypeptide comprises the amino acid sequence set forth in SEQ ID NO: 17, or an amino acid sequence having at least 90% identity to the amino acid sequence set forth in SEQ ID NO: 17.
E28. The method of embodiment 27, wherein the IL-15 polypeptide is encoded by a nucleotide sequence comprising the nucleotide sequence set forth SEQ ID NO: 122, or a nucleotide sequence having at least 80% identity to the nucleotide sequence set forth SEQ ID NO: 122.
E29. The method of any one of embodiments 1-28, wherein the IL-15Rα polypeptide comprises the amino acid sequence set forth in SEQ ID NO: 13, or an amino acid sequence having at least 90% identity to the amino acid sequence set forth in SEQ ID NO: 13.
E30. The method of embodiment 29, wherein the IL-15Rα polypeptide is encoded by a nucleotide sequence comprising the nucleotide sequence set forth SEQ ID NO: 22, or a nucleotide sequence having at least 80% identity to the nucleotide sequence set forth SEQ ID NO: 22.
E31. The method of any one of embodiments 1-22, wherein the IL-15 polypeptide operably linked to an IL-15Rα polypeptide comprises the amino acid sequence set forth in any one of SEQ ID NOs: 23, 27 and 123, or an amino acid sequence having at least 90% identity to the amino acid sequence set forth in any one of SEQ ID NOs: 23, 27 and 123.
E32. The method of embodiment 31, wherein the IL-15 polypeptide operably linked to an IL-15Rα polypeptide is encoded by a nucleotide sequence comprising the nucleotide sequence set forth in any one of SEQ ID NOs: 24-26, 28-30 and 124-126, or a nucleotide sequence having at least 80% identity to the nucleotide sequence set forth in any one of SEQ ID NOs: 24-26, 28-30 and 124-126.
E33. The method of any one of the preceding embodiments, wherein each mRNA comprises a 3′ untranslated region (UTR).
E34. The method of embodiment 33, wherein the 3′UTR comprises at least one microRNA (miR) binding site.
E35. The method of embodiment 34, wherein the at least one miR binding site is a miR-122 binding site.
E36. The method of embodiment 35, wherein the miR-122 binding site is a miR-122-3p or a miR-122-5p binding site.
E37. The method of embodiment 36, wherein the miR-122-5p binding site comprises the nucleotide sequence set forth in SEQ ID NO: 83, and wherein the miR-122-3p binding site comprises the nucleotide sequence set forth in SEQ ID NO: 74.
E38. The method of any one of embodiments 1-32, wherein each mRNA comprises a 3′UTR comprising the nucleotide sequence set forth in SEQ ID NO: 77 or SEQ ID NO: 121.
E39. The method of any one of the preceding embodiments, wherein each mRNA comprises a 5′ untranslated region (UTR).
E40. The method of embodiment 39, wherein the 5′UTR comprises the nucleotide sequence set forth in SEQ ID NO: 12 or SEQ ID NO: 76.
E41. The method of any one of the preceding embodiments, wherein each mRNA includes at least one chemical modification.
E42. The method of embodiment 41, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
E43. The method of any one of embodiments 1-40, wherein at least 95% of uridines in each mRNA are N1-methylpseudouridine.
E44. The method embodiment 43, wherein at least 99% of uridines in each mRNA are N1-methylpseudouridine.
E45. The method of embodiment 43, wherein 100% of uridines in each mRNA are N1-methylpseudouridine.
E46. The method of any one of the preceding embodiments, wherein each mRNA is formulated in the same lipid nanoparticle.
E47. The method of any one of embodiments 1-45, wherein each mRNA is formulated in a separate lipid nanoparticle.
E48. The method of embodiment 46 or embodiment 47, wherein the lipid nanoparticle comprises a molar ratio of about 20-60% ionizable amino lipid: 5-25% phospholipid: 25-55% structural lipid; and 0.5-15% PEG-modified lipid.
E49. The method of embodiment 46 or embodiment 47, wherein the lipid nanoparticle comprises a molar ratio of about 50% ionizable lipid: about 10% phospholipid: about 38.5% sterol; and about 1.5% PEG-modified lipid.
E50). The method of embodiment 46 or embodiment 47, wherein the lipid nanoparticle comprises a molar ratio of 50:38.5:10:1.5 of ionizable lipid: cholesterol: DSPC: PEG-modified lipid.
E51. The method of any one of embodiments 48-50, wherein the ionizable lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).
E52. The method of any one of embodiments 48-50, wherein the ionizable lipid comprises Compound II.
E53. The method of embodiment 46 or embodiment 47, wherein the lipid nanoparticle comprises a molar ratio of about 20-60% Compound II: 5-25% phospholipid: 25-55% cholesterol; and 0.5-15% PEG-modified lipid.
E54. The method of embodiment 53, wherein the lipid nanoparticle comprises a molar ratio of about 50% Compound II: about 10% phospholipid: about 38.5% cholesterol; and about 1.5% PEG-modified lipid.
E55. The method of any one of embodiments 48-54, wherein the PEG-modified lipid is PEG-DMG or Compound I.
E56. The method of embodiment 46 or embodiment 47, wherein the lipid nanoparticle comprises a molar ratio of 50:38.5:10:1.5 of Compound II: cholesterol: phospholipid: Compound I, or of Compound II: cholesterol: DSPC: Compound I.
E57. The method of embodiment 46 or embodiment 47, wherein the lipid nanoparticle comprises a molar ratio of 40:38.5:20:1.5 of Compound II: cholesterol: phospholipid: Compound I, or of Compound II: cholesterol: DSPC: Compound I.
E58. A method of any one of embodiments 1-57, wherein the myeloid malignancy is selected from the group consisting of myelodysplastic syndrome (MDS), myeloproliferative disorder (MPD) and acute myeloid leukemia (AML).
E59. The method of embodiment 58, wherein the myeloid malignancy is AML.
E60. The method of any one of embodiments 1-59, wherein the at least two mRNAs are administered intratumorally.
E61. The method of any one of embodiments 1-59, wherein the at least two mRNAs are administered intravenously.
E62. The method of any one of embodiments 1-61, comprising administering a checkpoint inhibitor polypeptide.
E63. The method of embodiment 62, wherein the checkpoint inhibitor polypeptide inhibits PD-1, PD-L¹, CTLA-4, or a combination thereof.
E64. The method of embodiment 63, wherein the checkpoint inhibitor polypeptide is an antibody or an mRNA encoding the antibody.
E65. The method of embodiment 64, wherein the antibody is an anti-CTLA-4 antibody or antigen-binding fragment thereof that specifically binds CTLA-4, an anti-PD-1 antibody or antigen-binding fragment thereof that specifically binds PD-1, an anti-PD-L¹ antibody or antigen-binding fragment thereof that specifically binds PD-L¹, and a combination thereof.
E66. The method of embodiment 65, wherein the anti-PD-L¹ antibody is atezolizumab, avelumab, or durvalumab, wherein the anti-CTLA-4 antibody is tremelimumab or ipilimumab, and wherein the anti-PD-1 antibody is nivolumab or pembrolizumab.
E67. A lipid nanoparticle comprising at least two encapsulated messenger RNAs (mRNAs), wherein the at least two mRNAs are selected from the group consisting of:

(i) an mRNA encoding an OX40L polypeptide;

(ii) an mRNA encoding an IL-15 polypeptide operably linked to an IL-15Rα polypeptide;

(iii) an mRNA encoding an IL-15 polypeptide and an mRNA encoding an IL-15Rα polypeptide; and

(iv) an mRNA encoding an IL-12 polypeptide operably linked to a membrane domain comprising a transmembrane domain.
E68. The lipid nanoparticle of embodiment 67, wherein the at least two mRNAs are selected from the group consisting of:

(i) an mRNA encoding an OX40L polypeptide and an mRNA encoding an IL-12 polypeptide operably linked to a membrane domain comprising a transmembrane domain;

(ii) an mRNA encoding an OX40L polypeptide, an mRNA encoding an IL-15 polypeptide and an mRNA encoding an IL-15Rα polypeptide;

(iii) an mRNA encoding an OX40L polypeptide and an mRNA encoding an IL-15 polypeptide operably linked to an IL-15Rα polypeptide;

(iv) an mRNA encoding an IL-15 polypeptide, an mRNA encoding an IL-15Rα polypeptide, and an mRNA encoding an IL-12 polypeptide operably linked to a membrane domain comprising a transmembrane domain;

(v) an mRNA encoding an IL-15 polypeptide operably linked to an IL-15Rα polypeptide, and an mRNA encoding an IL-12 polypeptide operably linked to a membrane domain comprising a transmembrane domain;

(vi) an mRNA encoding an OX40L polypeptide, an mRNA encoding an IL-15 polypeptide, an mRNA encoding an IL-15Rα polypeptide and an mRNA encoding an IL-12 polypeptide operably linked to a membrane domain comprising a transmembrane domain; and

(vii) an mRNA encoding an OX40L polypeptide, an mRNA encoding an IL-15 polypeptide operably linked to an IL-15Rα polypeptide, and an mRNA encoding an IL-12 polypeptide operably linked to a membrane domain comprising a transmembrane domain.
E69. A lipid nanoparticle comprising: an mRNA encoding an OX40L polypeptide, an mRNA encoding an IL-15 polypeptide, an mRNA encoding an IL-15Rα polypeptide and an mRNA encoding an IL-12 polypeptide operably linked to a membrane domain comprising a transmembrane domain.
E70. A lipid nanoparticle comprising:

(i) an mRNA encoding a human OX40L polypeptide, wherein the human OX40L polypeptide comprises the amino acid sequence set forth in SEQ ID NO: 1;

(ii) an mRNA encoding a human IL-15 polypeptide, wherein the human IL-15 polypeptide comprises the amino acid sequence set forth in SEQ ID NO: 17;

(iii) an mRNA encoding a human IL-15Rα polypeptide, wherein the human IL-15Rα polypeptide comprises the amino acid sequence set forth in SEQ ID NO: 13; and

(iv) an mRNA encoding a human IL-12 polypeptide operably linked to a membrane domain comprising a transmembrane domain, wherein the human IL-12 polypeptide comprises the amino acid sequence set forth in SEQ ID NO: 61, wherein the lipid nanoparticle comprises a molar ratio of about 20-60% ionizable amino lipid: 5-25% phospholipid: 25-55% structural lipid; and 0.5-15% PEG-modified lipid.
E71. A lipid nanoparticle comprising:

(i) an mRNA encoding a human OX40L polypeptide, wherein the mRNA comprises the nucleotide sequence set forth in SEQ ID NO: 11, or a nucleotide sequence having at least 80% identity to the nucleotide sequence set forth in SEQ ID NO: 11;

(ii) an mRNA encoding a human IL-15 polypeptide, wherein the mRNA comprises the nucleotide sequence set forth in SEQ ID NO: 122, or a nucleotide sequence having at least 80% identity to the nucleotide sequence set forth in SEQ ID NO: 122;

(iii) an mRNA encoding a human IL-15Rα polypeptide, wherein the mRNA comprises the nucleotide sequence set forth in SEQ ID NO: 22, or a nucleotide sequence having at least 80% identity to the nucleotide sequence set forth in SEQ ID NO: 22; and

(iv) an mRNA encoding a human IL-12 polypeptide operably linked to a membrane domain comprising a transmembrane domain, wherein the mRNA comprises the nucleotide sequence set forth in SEQ ID NO: 60, or a nucleotide sequence having at least 80% identity to the nucleotide sequence set forth in SEQ ID NO: 60,

wherein the lipid nanoparticle comprises a molar ratio of about 20-60% ionizable amino lipid: 5-25% phospholipid: 25-55% structural lipid; and 0.5-15% PEG-modified lipid.
E72. The lipid nanoparticle of any one of embodiments 68-71, comprising a 1:1:1:1 ratio of OX40L:IL-15:IL-15Rα:IL-12.
E73. The lipid nanoparticle of any one of embodiments 67-72, formulated for intratumoral delivery.
E74. The lipid nanoparticle of any one of embodiments 67-72, formulated for intravenous delivery.
E75. The lipid nanoparticle of any one of embodiments 67-69, wherein the lipid nanoparticle comprises a molar ratio of about 20-60% ionizable amino lipid: 5-25% phospholipid: 25-55% structural lipid; and 0.5-15% PEG-modified lipid.
E76. The lipid nanoparticle of any one of embodiments 70-75, wherein the lipid nanoparticle comprises a molar ratio of about 50% ionizable lipid: about 10% phospholipid: about 38.5% sterol; and about 1.5% PEG-modified lipid.
E77. The lipid nanoparticle of any one of embodiments 67-74, wherein the lipid nanoparticle comprises a molar ratio of 50:38.5:10:1.5 of ionizable lipid: cholesterol: DSPC: PEG-modified lipid.
E78. The lipid nanoparticle of any one of embodiments 70-77, wherein the ionizable lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).
E79. The lipid nanoparticle of any one of embodiments 70-78, wherein the ionizable lipid comprises Compound II.
E80. The lipid nanoparticle of any one of embodiments 67-74, wherein the lipid nanoparticle comprises a molar ratio of about 20-60% Compound II: 5-25% phospholipid: 25-55% cholesterol; and 0.5-15% PEG-modified lipid.
E81. The lipid nanoparticle of embodiment 80, wherein the lipid nanoparticle comprises a molar ratio of about 50% Compound II: about 10% phospholipid: about 38.5% cholesterol; and about 1.5% PEG-modified lipid.
E82. The lipid nanoparticle of any one of embodiments 70-81, wherein the PEG-modified lipid is PEG-DMG or Compound I.
E83. The lipid nanoparticle of any one of embodiments 67-82, wherein the lipid nanoparticle comprises a molar ratio of 50:38.5:10:1.5 of Compound II: cholesterol: phospholipid: Compound I, or of Compound II: cholesterol: DSPC: Compound I.
E84. The lipid nanoparticle of any one of embodiments 67-82, wherein the lipid nanoparticle comprises a molar ratio of 40:38.5:20:1.5 of Compound II: cholesterol: phospholipid: Compound I, or of Compound II: cholesterol: DSPC: Compound I.
E85. A method for treating a myeloid malignancy in a subject in need thereof, the method comprising administering to the subject the lipid nanoparticle of any one of embodiments 67-84.
E86. The method of embodiment 85, further comprising administering an immune checkpoint inhibitor polypeptide.
E87. The method of embodiment 86, wherein the checkpoint inhibitor polypeptide inhibits PD-1, PD-L¹, CTLA-4, or a combination thereof.
E88. The method of embodiment 87, wherein the checkpoint inhibitor polypeptide is an antibody or an mRNA encoding the antibody.
E89. The method of embodiment 88, wherein the antibody is an anti-CTLA-4 antibody or antigen-binding fragment thereof that specifically binds CTLA-4, an anti-PD-1 antibody or antigen-binding fragment thereof that specifically binds PD-1, an anti-PD-L¹ antibody or antigen-binding fragment thereof that specifically binds PD-L¹, and a combination thereof.
E90. The method of embodiment 89, wherein the anti-PD-L¹ antibody is atezolizumab, avelumab, or durvalumab, wherein the anti-CTLA-4 antibody is tremelimumab or ipilimumab, and wherein the anti-PD-1 antibody is nivolumab or pembrolizumab.
E91. The lipid nanoparticle of any one of embodiments 67-84, and an optional pharmaceutically acceptable carrier, for use in treating or delaying progression of a myeloid malignancy in an individual, wherein treatment comprises administration of the lipid nanoparticle.
E92. The lipid nanoparticle of any one of embodiments 67-84, and an optional pharmaceutically acceptable carrier, for use in treating or delaying progression of a myeloid malignancy in an individual, wherein treatment comprises administration of the lipid nanoparticle in combination with a composition comprising an immune checkpoint inhibitory polypeptide, and an optional pharmaceutically acceptable carrier.
E93. Use of a lipid nanoparticle of any one of embodiments 67-84, and an optional pharmaceutically acceptable carrier, in the manufacture of a medicament for treating or delaying progression of a myeloid malignancy in an individual, wherein the medicament comprises the lipid nanoparticle, and an optional pharmaceutically acceptable carrier, and wherein the treatment comprises administration of the medicament.
E94. Use of a lipid nanoparticle of any one of embodiments 67-84, and an optional pharmaceutically acceptable carrier, in the manufacture of a medicament for treating or delaying progression of a myeloid malignancy in an individual, wherein the medicament comprises the lipid nanoparticle, and an optional pharmaceutically acceptable carrier, and wherein the treatment comprises administration of the medicament in combination with a composition comprising an immune checkpoint inhibitor polypeptide, and an optional pharmaceutically acceptable carrier.
E95. A kit comprising a container comprising the lipid nanoparticle of any one of embodiments 67-84, and an optional pharmaceutically acceptable carrier, and a package insert comprising instructions for administration of the lipid nanoparticle for treating or delaying progression of a myeloid malignancy in an individual.
E96. The kit of embodiment 95, wherein the package insert further comprises instructions for administration of the lipid nanoparticle in combination with a composition comprising an immune checkpoint inhibitor polypeptide, and an optional pharmaceutically acceptable carrier, for treating or delaying progression of a myeloid malignancy in an individual.
E97. A kit comprising a container comprising the lipid nanoparticle of any one of embodiments 67-84, and an optional pharmaceutically acceptable carrier, and a package insert comprising instructions for administration of the medicament alone, or in combination with a composition comprising an immune checkpoint inhibitor polypeptide, and an optional pharmaceutically acceptable carrier, for treating or delaying progression of a myeloid malignancy in an individual.
E98. A method for enhancing an immune response in a subject, comprising administering to the subject the lipid nanoparticle of any one of embodiments 67-84.
E99. A method for enhancing T cell activation in a subject, comprising administering to the subject the lipid nanoparticle of any one of embodiments 67-84.
E100. A method for enhancing NK cell activation in a subject, comprising administering to the subject the lipid nanoparticle of any one of embodiments 67-84.
E101. The method of any one of embodiments 98-100, wherein the subject has a myeloid malignancy.
E102. The method of any one of embodiments 98-101, further comprising administering an immune checkpoint inhibitor polypeptide.
E103. A method of treating cancer in a subject in need thereof, the method comprising administering to the subject at least two messenger RNAs (mRNAs) selected from the group consisting of:

(i) an mRNA encoding an OX40L polypeptide;

(ii) an mRNA encoding an IL-15 polypeptide operably linked to an IL-15Rα polypeptide;

(iii) an mRNA encoding an IL-15 polypeptide and an mRNA encoding an IL-15Rα polypeptide; and

(iv) an mRNA encoding an IL-12 polypeptide operably linked to a membrane domain comprising a transmembrane domain.
E104. The method of embodiment 103, wherein the at least two mRNAs are selected from the group consisting of:

(i) an mRNA encoding an OX40L polypeptide and an mRNA encoding an IL-12 polypeptide operably linked to a membrane domain comprising a transmembrane domain;

(ii) an mRNA encoding an OX40L polypeptide, an mRNA encoding an IL-15 polypeptide and an mRNA encoding an IL-15Rα polypeptide;

(iii) an mRNA encoding an OX40L polypeptide and an mRNA encoding an IL-15 polypeptide operably linked to an IL-15Rα polypeptide;

(iv) an mRNA encoding an IL-15 polypeptide, an mRNA encoding an IL-15Rα polypeptide, and an mRNA encoding an IL-12 polypeptide operably linked to a membrane domain comprising a transmembrane domain;

(v) an mRNA encoding an IL-15 polypeptide operably linked to an IL-15Rα polypeptide, and an mRNA encoding an IL-12 polypeptide operably linked to a membrane domain comprising a transmembrane domain;

(vi) an mRNA encoding an OX40L polypeptide, an mRNA encoding an IL-15 polypeptide, an mRNA encoding an IL-15Rα polypeptide and an mRNA encoding an IL-12 polypeptide operably linked to a membrane domain comprising a transmembrane domain; and

(vii) an mRNA encoding an OX40L polypeptide, an mRNA encoding an IL-15 polypeptide operably linked to an IL-15Rα polypeptide, and an mRNA encoding an IL-12 polypeptide operably linked to a membrane domain comprising a transmembrane domain.
E105. The method of embodiment 103 or embodiment 104, wherein the OX40L polypeptide comprises the amino acid sequence set forth in SEQ ID NO: 1, or an amino acid sequence having at least 90% identity to the amino acid sequence set forth in SEQ ID NO: 1.
E106. The method of embodiment 105, wherein the OX40L polypeptide is encoded by a nucleotide sequence comprising the nucleotide sequence set forth SEQ ID NO: 11, or a nucleotide sequence having at least 80% identity to the nucleotide sequence set forth SEQ ID NO: 11.
E107. The method of any one of embodiments 103-106, wherein the IL-12 polypeptide comprises an IL-12 p40 subunit (IL-12B) polypeptide operably linked to an IL-12 p35 subunit (IL-12A) polypeptide.
E108. The method of embodiment 107, wherein the IL-12B polypeptide is operably linked to the IL-12A polypeptide by a peptide linker.
E109. The method of embodiment 108, wherein the IL-12B polypeptide is located at the 5′ terminus of the IL-12A polypeptide, or the 5′ terminus of the peptide linker; or wherein the IL-12A polypeptide is located at the 5′ terminus of the IL-12B polypeptide, or the 5′ terminus of the peptide linker.
E110. The method of any one of embodiments 103-109, wherein the IL-12 polypeptide transmembrane domain comprises a transmembrane domain derived from a Type I integral membrane protein.
E111. The method of any one of embodiments 103-109, wherein the IL-12 polypeptide transmembrane domain is selected from the group consisting of: a Cluster of Differentiation 8 (CD8) transmembrane domain, a Platelet-Derived Growth Factor Receptor (PDGFR) transmembrane domain, and a Cluster of Differentiation 80 (CD80) transmembrane domain.
E112. The method of embodiment 111, the PDGFR-beta transmembrane domain comprises the amino acid sequence set forth in SEQ ID NO: 42 the CD8 transmembrane domain comprises the amino acid sequence set forth in SEQ ID NO: 41; and the CD80 transmembrane domain comprises the amino acid sequence set forth in SEQ ID NO: 43.
E113. The method of any one of embodiments 103-112, wherein the IL-12 polypeptide membrane domain comprises an intracellular domain.
E114. The method of embodiment E113, wherein the intracellular domain is derived from the same polypeptide as the transmembrane domain, or wherein the intracellular domain is derived from a different polypeptide than the transmembrane domain is derived from.
E115. The method of embodiment 113, wherein the intracellular domain is selected from the group consisting of: a PDGFR intracellular domain, a truncated PDGFR intracellular domain, and a CD80 intracellular domain.
E116. The method of embodiment 115, wherein the intracellular domain is a PDGFR intracellular domain comprising a PDGFR-beta intracellular domain comprising the amino acid sequence set forth in SEQ ID NO: 48.
E117. The method of embodiment 115, wherein the intracellular domain is a truncated PDGFR intracellular domain comprising a PDGFR-beta intracellular domain truncated at E570 or G739.
E118. The method of embodiment 117, wherein the truncated PDGFR-beta intracellular domain truncated at E570 comprises the amino acid sequence set forth in SEQ ID NO: 49, and wherein the truncated PDGFR-beta transmembrane truncated at G739 comprises the amino acid sequence set forth in SEQ ID NO: 50.
E119. The method of embodiment 115, wherein the intracellular domain is a CD80 intracellular domain comprising the amino acid sequence set forth in SEQ ID NO: 47.
E120. The method of any one of embodiments 103-119, wherein the IL-12 polypeptide membrane domain comprises:

(i) a PDGFR-beta transmembrane domain and a PDGFR-beta intracellular domain;

(ii) a PDGFR-beta transmembrane domain and a truncated PDGFR-beta intracellular domain truncated at E570;

(iii) a PDGFR-beta transmembrane domain and a truncated PDGFR-beta intracellular domain truncated at G739; or

(iv) a CD80 transmembrane domain and a CD80 intracellular domain.
E121. The method of any one of embodiments 103-120, wherein the IL-12 polypeptide membrane domain is operably linked to the IL-12A polypeptide by a peptide linker, or wherein the IL-12 polypeptide membrane domain is operably linked to the IL-12B polypeptide by a peptide linker.
E122. The method of any one of embodiments 103-121, wherein the IL-15Rα polypeptide comprises a sushi domain.
E123. The method of embodiment 122, wherein the IL-15Rα polypeptide further comprises an intracellular domain and a transmembrane domain.
E124. The method of embodiment 123, wherein the intracellular domain and the transmembrane domain are derived from IL-15Rα.
E125. The method of embodiment 123, wherein the intracellular domain and the transmembrane domain are derived from a heterologous polypeptide.
E126. The method of any one of embodiments 103-125, wherein the IL-15 polypeptide comprises the amino acid sequence set forth in SEQ ID NO: 17, or an amino acid sequence having at least 90% identity to the amino acid sequence set forth in SEQ ID NO: 17.
E127. The method of embodiment 126, wherein the IL-15 polypeptide is encoded by a nucleotide sequence comprising the nucleotide sequence set forth SEQ ID NO: 122, or a nucleotide sequence having at least 80% identity to the nucleotide sequence set forth SEQ ID NO: 122.
E128. The method of any one of embodiments 103-127 wherein the IL-15Rα polypeptide comprises the amino acid sequence set forth in SEQ ID NO: 13, or an amino acid sequence having at least 90% identity to the amino acid sequence set forth in SEQ ID NO: 13.
E129. The method of embodiment 128, wherein the IL-15Rα polypeptide is encoded by a nucleotide sequence comprising the nucleotide sequence set forth SEQ ID NO: 22, or a nucleotide sequence having at least 80% identity to the nucleotide sequence set forth SEQ ID NO: 22.
E130. The method of any one of embodiments 103-121, wherein the IL-15 polypeptide operably linked to an IL-15Rα polypeptide comprises the amino acid sequence set forth in any one of SEQ ID NOs: 23, 27 and 123, or an amino acid sequence having at least 90% identity to the amino acid sequence set forth in any one of SEQ ID NOs: 23, 27 and 123.
E131. The method of embodiment 130, wherein the IL-15 polypeptide operably linked to an IL-15Rα polypeptide is encoded by a nucleotide sequence comprising the nucleotide sequence set forth in any one of SEQ ID NOs: 24-26, 28-30 and 124-126, or a nucleotide sequence having at least 80% identity to the nucleotide sequence set forth in any one of SEQ ID NOs: 24-26, 28-30 and 124-126.
E132. The method of any one of embodiments 103-131, wherein the cancer is a solid tumor.
E133. The method of embodiment 132, wherein the solid tumor comprises an immunosuppressive tumor microenvironment.
E134. The method of any one of embodiments 132-133, wherein the solid tumor is unresponsive to immune checkpoint inhibitor therapy.
E135. The method of any one of embodiments 103-131, wherein the cancer is a disseminated cancer.
E136. The method of embodiment 135, wherein the disseminated cancer is a hematological cancer.
E137. The method of embodiment 135, wherein the disseminated cancer is a myeloid malignancy.
E138. The method of embodiment 137, wherein the myeloid malignancy is selected from the group consisting of myelodysplastic syndrome (MDS), myeloproliferative disorder (MPD) and acute myeloid leukemia (AML).
E139. The method of embodiment 138, wherein the myeloid malignancy is AML.
E140. The method of any one of embodiments 103-139, wherein the at least two mRNAs are administered intratumorally.
E141. The method of any one of embodiments 103-139, wherein the at least two mRNAs are administered intravenously.
E142. The method of any one of embodiments 103-131, wherein the cancer is a solid tumor and wherein the at least two mRNAs are administered intratumorally.
E143. The method of any one of embodiments 103-131, wherein the cancer is a disseminated cancer and wherein the at least two mRNAs are administered intravenously.
E144. A method of treating a disseminated cancer in a human patient, comprising systemically administering to the patient:

(i) a first mRNA encoding human OX40L; and

(ii) at least one second mRNA encoding an immune potentiator, wherein the immune potentiator is a cell-associated cytokine that activates T cells, NK cells, or both T cells and NK cells,
wherein the first mRNA and second mRNA are encapsulated in the same or different lipid nanoparticles.
E145. A method of treating a disseminated cancer in a human patient, comprising systemically administering to the patient a pharmaceutical composition comprising a lipid nanoparticle (LNP) and a pharmaceutically acceptable carrier, wherein the LNP comprises:

(i) a first mRNA encoding human OX40L; and

(ii) at least one second mRNA encoding an immune potentiator, wherein the immune potentiator is a cell-associated cytokine that activates T cells, NK cells, or both T cells and NK cells.
E146. A method of treating a disseminated cancer in a human patient, comprising administering to the patient a dosing regimen comprising:

(i) a first fractionated dose of a pharmaceutical composition comprising a first mRNA encoding human OX40L, and at least one second mRNA encoding an immune potentiator, wherein the immune potentiator is a cell-associated cytokine that activates T cells, NK cells, or both T cells and NK cells, and

(ii) at least one second fractionated dose of the pharmaceutical composition, wherein the first and second fractionated doses increase exposure to the mRNA encoded polypeptides in the patient relative to a single dose of the same amount of mRNA during the same dosing interval, thereby treating the disseminated cancer in the patient.
E147. The method of embodiment 146, wherein the first fractionated dose and second fractionated dose enhance anti-tumor efficacy of the treatment relative to a single dose of the same amount of mRNA.
E148. The method of any one of embodiments 146 and 147, wherein the first fractionated dose and second fractionated dose enhance anti-tumor efficacy with reduced toxicity and better tolerability.
E149. The method of any one of embodiments 144-146, wherein the OX40L polypeptide comprises the amino acid sequence set forth in SEQ ID NO: 1, or an amino acid sequence having at least 90% identity to the amino acid sequence set forth in SEQ ID NO: 1.
E150. The method of embodiment 149, wherein the OX40L polypeptide is encoded by a nucleotide sequence comprising the nucleotide sequence set forth SEQ ID NO: 11, or a nucleotide sequence having at least 80% identity to the nucleotide sequence set forth SEQ ID NO: 11.
E151. The method of any one of embodiments 144-150, wherein the cell-associated cytokine is a human IL-12 polypeptide operably linked to a membrane domain comprising a transmembrane domain.
E152. The method of any one of embodiments 144-150, wherein the cell-associated cytokine is a trans-presented human IL-15.
E153. The method of embodiment 152, wherein the trans-presented human IL-15 is a human IL-15 polypeptide operably linked to a human IL-15Rα polypeptide.
E154. The method of embodiment 152, wherein the trans-presented human IL-15 is encoded by a first mRNA encoding a human IL-15 polypeptide and a second mRNA encoding a IL-15Rα polypeptide.
E155. The method of any one of embodiments 150-154, comprising administering a third mRNA encoding a second immune potentiator, wherein the immune potentiator is a cell-associated cytokine that activates T cells, NK cells, or both T cells and NK cells.
E156. The method of embodiment 155, wherein the second immune potentiator is a human IL-12 polypeptide operably linked to a membrane domain comprising a transmembrane domain.
E157. The method of any one of embodiments 150 and 156, wherein the IL-12 polypeptide comprises an IL-12 p40 subunit (IL-12B) polypeptide operably linked to an IL-12 p35 subunit (IL-12A) polypeptide.
E158. The method of embodiment 157, wherein the IL-12B polypeptide is operably linked to the IL-12A polypeptide by a peptide linker.
E159. The method of embodiment 158, wherein the IL-12B polypeptide is located at the 5′ terminus of the IL-12A polypeptide, or the 5′ terminus of the peptide linker; or wherein the IL-12A polypeptide is located at the 5′ terminus of the IL-12B polypeptide, or the 5′ terminus of the peptide linker.
E160. The method of any one of embodiments 150 and 155-159, wherein the IL-12 polypeptide transmembrane domain comprises a transmembrane domain derived from a Type I integral membrane protein.
E161. The method of any one of embodiments 150 and 155-159, wherein the IL-12 polypeptide transmembrane domain is selected from the group consisting of: a Cluster of Differentiation 8 (CD8) transmembrane domain, a Platelet-Derived Growth Factor Receptor (PDGFR) transmembrane domain, and a Cluster of Differentiation 80 (CD80) transmembrane domain.
E162. The method of embodiment 161, the PDGFR-beta transmembrane domain comprises the amino acid sequence set forth in SEQ ID NO: 42 the CD8 transmembrane domain comprises the amino acid sequence set forth in SEQ ID NO: 41; and the CD80 transmembrane domain comprises the amino acid sequence set forth in SEQ ID NO: 43.
E163. The method of any one of embodiments 150 and 155-162, wherein the IL-12 polypeptide membrane domain comprises an intracellular domain.
E164. The method of embodiment 163, wherein the intracellular domain is derived from the same polypeptide as the transmembrane domain, or wherein the intracellular domain is derived from a different polypeptide than the transmembrane domain is derived from.
E165. The method of embodiment 163, wherein the intracellular domain is selected from the group consisting of: a PDGFR intracellular domain, a truncated PDGFR intracellular domain, and a CD80 intracellular domain.
E166. The method of embodiment 165, wherein the intracellular domain is a PDGFR intracellular domain comprising a PDGFR-beta intracellular domain comprising the amino acid sequence set forth in SEQ ID NO: 48.
E167. The method of embodiment 165, wherein the intracellular domain is a truncated PDGFR intracellular domain comprising a PDGFR-beta intracellular domain truncated at E570 or G739.
E168. The method of embodiment 167, wherein the truncated PDGFR-beta intracellular domain truncated at E570 comprises the amino acid sequence set forth in SEQ ID NO: 49, and wherein the truncated PDGFR-beta transmembrane truncated at G739 comprises the amino acid sequence set forth in SEQ ID NO: 50.
E169. The method of embodiment 165, wherein the intracellular domain is a CD80 intracellular domain comprising the amino acid sequence set forth in SEQ ID NO: 47.
E170. The method of any one of embodiments 148 and 153-159, wherein the IL-12 polypeptide membrane domain comprises:

(i) a PDGFR-beta transmembrane domain and a PDGFR-beta intracellular domain;

(ii) a PDGFR-beta transmembrane domain and a truncated PDGFR-beta intracellular domain truncated at E570;

(iii) a PDGFR-beta transmembrane domain and a truncated PDGFR-beta intracellular domain truncated at G739; or

(iv) a CD80 transmembrane domain and a CD80 intracellular domain.
E171. The method of any one of embodiments 150 and 155-170, wherein the IL-12 polypeptide membrane domain is operably linked to the IL-12A polypeptide by a peptide linker, or wherein the IL-12 polypeptide membrane domain is operably linked to the IL-12B polypeptide by a peptide linker.
E172. The method of any one of embodiments 153-171, wherein the IL-15Rα polypeptide comprises a sushi domain.
E173. The method of embodiment 172, wherein the IL-15Rα polypeptide further comprises an intracellular domain and a transmembrane domain.
E174. The method of embodiment 173, wherein the intracellular domain and the transmembrane domain are derived from IL-15Rα.
E175. The method of embodiment 173, wherein the intracellular domain and the transmembrane domain are derived from a heterologous polypeptide.
E176. The method of any one of embodiments 153-175, wherein the IL-15 polypeptide comprises the amino acid sequence set forth in SEQ ID NO: 17, or an amino acid sequence having at least 90% identity to the amino acid sequence set forth in SEQ ID NO: 17.
E177. The method of embodiment 176, wherein the IL-15 polypeptide is encoded by a nucleotide sequence comprising the nucleotide sequence set forth SEQ ID NO: 122, or a nucleotide sequence having at least 80% identity to the nucleotide sequence set forth SEQ ID NO: 122.
E178. The method of any one of embodiments 153-177, wherein the IL-15Rα polypeptide comprises the amino acid sequence set forth in SEQ ID NO: 13, or an amino acid sequence having at least 90% identity to the amino acid sequence set forth in SEQ ID NO: 13.
E179. The method of embodiment 178, wherein the IL-15Rα polypeptide is encoded by a nucleotide sequence comprising the nucleotide sequence set forth SEQ ID NO: 22, or a nucleotide sequence having at least 80% identity to the nucleotide sequence set forth SEQ ID NO: 22.
E180. The method of any one of embodiments 152 and 154-171, wherein the IL-15 polypeptide operably linked to an IL-15Rα polypeptide comprises the amino acid sequence set forth in any one of SEQ ID NOs: 23, 27 and 123, or an amino acid sequence having at least 90% identity to the amino acid sequence set forth in any one of SEQ ID NOs: 23, 27 and 123.
E181. The method of embodiment 180, wherein the IL-15 polypeptide operably linked to an IL-15Rα polypeptide is encoded by a nucleotide sequence comprising the nucleotide sequence set forth in any one of SEQ ID NOs: 24-26, 28-30 and 124-126, or a nucleotide sequence having at least 80% identity to the nucleotide sequence set forth in any one of SEQ ID NOs: 24-26, 28-30 and 124-126.
E182. The method of any one of the preceding embodiments, wherein each mRNA comprises a 3′ untranslated region (UTR).
E183. The method of embodiment 182, wherein the 3′UTR comprises at least one microRNA (miR) binding site.
E184. The method of embodiment 183, wherein the at least one miR binding site is a miR-122 binding site.
E185. The method of embodiment 184, wherein the miR-122 binding site is a miR-122-3p or a miR-122-5p binding site.
E186. The method of embodiment 185, wherein the miR-122-5p binding site comprises the nucleotide sequence set forth in SEQ ID NO: 83, and wherein the miR-122-3p binding site comprises the nucleotide sequence set forth in SEQ ID NO: 74.
E187. The method of any one of embodiments 103-181, wherein each mRNA comprises a 3′UTR comprising the nucleotide sequence set forth in SEQ ID NO: 77 or SEQ ID NO: 121
E188. The method of any one of the preceding embodiments, wherein each mRNA comprises a 5′ untranslated region (UTR).
E189. The method of embodiment 188, wherein the 5′UTR comprises the nucleotide sequence set forth in SEQ ID NO: 12 or SEQ ID NO: 76.
E190. The method of any one of the preceding embodiments, wherein each mRNA includes at least one chemical modification.
E191. The method of embodiment 190, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
E192. The method of any one of embodiments 103-189, wherein at least 95% of uridines in each mRNA are N1-methylpseudouridine.
E193. The method embodiment 192, wherein at least 99% of uridines in each mRNA are N1-methylpseudouridine.
E194. The method of embodiment 192, wherein 100% of uridines in each mRNA are N1-methylpseudouridine.
E195. The method of any one of the preceding embodiments, wherein each mRNA is formulated in the same lipid nanoparticle.
E196. The method of any one of embodiments 103-144 and 146-194, wherein each mRNA is formulated in a separate lipid nanoparticle.
E197. The method of any one of embodiments 195-196, wherein the lipid nanoparticle comprises a molar ratio of about 20-60% ionizable amino lipid: 5-25% phospholipid: 25-55% structural lipid; and 0.5-15% PEG-modified lipid.
E198. The method of any one of embodiments 195-196, wherein the lipid nanoparticle comprises a molar ratio of about 50% ionizable lipid: about 10% phospholipid: about 38.5% sterol; and about 1.5% PEG-modified lipid.
E199. The method of any one of embodiments 195-196, wherein the lipid nanoparticle comprises a molar ratio of 50:38.5:10:1.5 of ionizable lipid: cholesterol: DSPC: PEG-modified lipid.
E200. The method of any one of embodiments 197-199, wherein the ionizable lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).
E201. The method of any one of embodiments 197-199, wherein the ionizable lipid comprises Compound II.
E202. The method of any one of embodiments 195-196, wherein the lipid nanoparticle comprises a molar ratio of about 20-60% Compound II: 5-25% phospholipid: 25-55% cholesterol; and 0.5-15% PEG-modified lipid.
E203. The method of embodiment 202, wherein the lipid nanoparticle comprises a molar ratio of about 50% Compound II: about 10% phospholipid: about 38.5% cholesterol; and about 1.5% PEG-modified lipid.
E204. The method of any one of embodiments 197-203, wherein the PEG-modified lipid is PEG-DMG or Compound I.
E205. The method of any one of embodiments 195-196, wherein the lipid nanoparticle comprises a molar ratio of 50:38.5:10:1.5 of Compound II: cholesterol: phospholipid: Compound I, or of Compound II: cholesterol: DSPC: Compound I.
E206. The method of any one of embodiments 195-196, wherein the lipid nanoparticle comprises a molar ratio of 40:38.5:20:1.5 of Compound II: cholesterol: phospholipid: Compound I, or of Compound II: cholesterol: DSPC: Compound I.
E207. The method of any one of embodiments 103-206, comprising administering a checkpoint inhibitor polypeptide.
E208. The method of embodiment 207, wherein the checkpoint inhibitor polypeptide inhibits PD-1, PD-L¹, CTLA-4, or a combination thereof.
E209. The method of embodiment 208, wherein the checkpoint inhibitor polypeptide is an antibody or an mRNA encoding the antibody.
E210. The method of embodiment 209, wherein the antibody is an anti-CTLA-4 antibody or antigen-binding fragment thereof that specifically binds CTLA-4, an anti-PD-1 antibody or antigen-binding fragment thereof that specifically binds PD-1, an anti-PD-L¹ antibody or antigen-binding fragment thereof that specifically binds PD-L¹, and a combination thereof.
E211. The method of embodiment 210, wherein the anti-PD-L¹ antibody is atezolizumab, avelumab, or durvalumab, wherein the anti-CTLA-4 antibody is tremelimumab or ipilimumab, and wherein the anti-PD-1 antibody is nivolumab or pembrolizumab.
E212. A lipid nanoparticle comprising at least two encapsulated messenger RNAs (mRNAs), wherein the at least two mRNAs are selected from the group consisting of:

(i) an mRNA encoding an OX40L polypeptide;

(ii) an mRNA encoding an IL-15 polypeptide operably linked to an IL-15Rα polypeptide;

(iii) an mRNA encoding an IL-15 polypeptide and an mRNA encoding an IL-15Rα polypeptide; and

(iv) an mRNA encoding an IL-12 polypeptide.
E213. The lipid nanoparticle of embodiment 212, wherein the at least two mRNAs are selected from the group consisting of:

(i) an mRNA encoding an OX40L polypeptide and an mRNA encoding an IL-12 polypeptide;

(ii) an mRNA encoding an OX40L polypeptide, an mRNA encoding an IL-15 polypeptide and an mRNA encoding an IL-15Rα polypeptide;

(iii) an mRNA encoding an OX40L polypeptide and an mRNA encoding an IL-15 polypeptide operably linked to an IL-15Rα polypeptide;

(iv) an mRNA encoding an IL-15 polypeptide, an mRNA encoding an IL-15Rα polypeptide, and an mRNA encoding an IL-12 polypeptide;

(v) an mRNA encoding an IL-15 polypeptide operably linked to an IL-15Rα polypeptide, and an mRNA encoding an IL-12 polypeptide;

(vi) an mRNA encoding an OX40L polypeptide, an mRNA encoding an IL-15 polypeptide, an mRNA encoding an IL-15Rα polypeptide and an mRNA encoding an IL-12 polypeptide; and

(vii) an mRNA encoding an OX40L polypeptide, an mRNA encoding an IL-15 polypeptide operably linked to an IL-15Rα polypeptide, and an mRNA encoding an IL-12 polypeptide.
E214. A lipid nanoparticle comprising: an mRNA encoding an OX40L polypeptide, an mRNA encoding an IL-15 polypeptide, an mRNA encoding an IL-15Rα polypeptide and an mRNA encoding an IL-12 polypeptide.
E215. A lipid nanoparticle comprising:

(i) an mRNA encoding a human OX40L polypeptide, wherein the human OX40L polypeptide comprises the amino acid sequence set forth in SEQ ID NO: 1;

(ii) an mRNA encoding a human IL-15 polypeptide, wherein the human IL-15 polypeptide comprises the amino acid sequence set forth in SEQ ID NO: 17;

(iii) an mRNA encoding a human IL-15Rα polypeptide, wherein the human IL-15Rα polypeptide comprises the amino acid sequence set forth in SEQ ID NO: 13; and

(iv) an mRNA encoding a human IL-12 polypeptide, wherein the human IL-12 polypeptide comprises the amino acid sequence set forth in SEQ ID NO: 61, wherein the lipid nanoparticle comprises a molar ratio of about 20-60% ionizable amino lipid: 5-25% phospholipid: 25-55% structural lipid; and 0.5-15% PEG-modified lipid.
E216. A lipid nanoparticle comprising:

(i) an mRNA encoding a human OX40L polypeptide, wherein the mRNA comprises the nucleotide sequence set forth in SEQ ID NO: 11, or a nucleotide sequence having at least 80% identity to the nucleotide sequence set forth in SEQ ID NO: 11;

(ii) an mRNA encoding a human IL-15 polypeptide, wherein the mRNA comprises the nucleotide sequence set forth in SEQ ID NO: 122, or a nucleotide sequence having at least 80% identity to the nucleotide sequence set forth in SEQ ID NO: 122;

(iii) an mRNA encoding a human IL-15Rα polypeptide, wherein the mRNA comprises the nucleotide sequence set forth in SEQ ID NO: 22, or a nucleotide sequence having at least 80% identity to the nucleotide sequence set forth in SEQ ID NO: 22; and

(iv) an mRNA encoding a human IL-12 polypeptide, wherein the mRNA comprises the nucleotide sequence set forth in SEQ ID NO: 60, or a nucleotide sequence having at least 80% identity to the nucleotide sequence set forth in SEQ ID NO: 60, wherein the lipid nanoparticle comprises a molar ratio of about 20-60% ionizable amino lipid: 5-25% phospholipid: 25-55% structural lipid; and 0.5-15% PEG-modified lipid.
E217. The lipid nanoparticle of any one of embodiments 213-216, comprising a 1:1:1:1 ratio of OX40L:IL-15:IL-15Rα:IL-12.
E218. The lipid nanoparticle of any one of embodiments 212-217, formulated for intratumoral delivery.
E219. The lipid nanoparticle of any one of embodiments 212-217, formulated for intravenous delivery.
E220. The lipid nanoparticle of any one of embodiments 212-214, wherein the lipid nanoparticle comprises a molar ratio of about 20-60% ionizable amino lipid: 5-25% phospholipid: 25-55% structural lipid; and 0.5-15% PEG-modified lipid.
E221. The lipid nanoparticle of any one of embodiments 215-220, wherein the lipid nanoparticle comprises a molar ratio of about 50% ionizable lipid: about 10% phospholipid: about 38.5% sterol; and about 1.5% PEG-modified lipid.
E222. The lipid nanoparticle of any one of embodiments 212-219, wherein the lipid nanoparticle comprises a molar ratio of 50:38.5:10:1.5 of ionizable lipid: cholesterol: DSPC: PEG-modified lipid.
E223. The lipid nanoparticle of any one of embodiments 215-222, wherein the ionizable lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).
E224. The lipid nanoparticle of any one of embodiments 215-223, wherein the ionizable lipid comprises Compound II.
E225. The lipid nanoparticle of any one of embodiments 212-219, wherein the lipid nanoparticle comprises a molar ratio of about 20-60% Compound II: 5-25% phospholipid: 25-55% cholesterol; and 0.5-15% PEG-modified lipid.
E226. The lipid nanoparticle of embodiment 225, wherein the lipid nanoparticle comprises a molar ratio of about 50% Compound II: about 10% phospholipid: about 38.5% cholesterol; and about 1.5% PEG-modified lipid.
E227. The lipid nanoparticle of any one of embodiments 215-226, wherein the PEG-modified lipid is PEG-DMG or Compound I.
E228. The lipid nanoparticle of any one of embodiments 212-227, wherein the lipid nanoparticle comprises a molar ratio of 50:38.5:10:1.5 of Compound II: cholesterol: phospholipid: Compound I, or of Compound II: cholesterol: DSPC: Compound I.
E229. The lipid nanoparticle of any one of embodiments 212-227, wherein the lipid nanoparticle comprises a molar ratio of 40:38.5:20:1.5 of Compound II: cholesterol: phospholipid: Compound I, or of Compound II: cholesterol: DSPC: Compound I.
E230. A method for treating a cancer in a subject in need thereof, the method comprising administering to the subject the lipid nanoparticle of any one of embodiments 212-229.
E231. The method of embodiment 230, wherein the cancer is a disseminated cancer or a solid tumor.
E232. The method of embodiment 231, wherein the disseminated cancer is a myeloid malignancy.
E233. The method of any one of embodiments 230-232, further comprising administering an immune checkpoint inhibitor polypeptide.
E234. The method of embodiment 233, wherein the checkpoint inhibitor polypeptide inhibits PD-1, PD-L¹, CTLA-4, or a combination thereof.
E235. The method of embodiment 234, wherein the checkpoint inhibitor polypeptide is an antibody or an mRNA encoding the antibody.
E236. The method of embodiment 235, wherein the antibody is an anti-CTLA-4 antibody or antigen-binding fragment thereof that specifically binds CTLA-4, an anti-PD-1 antibody or antigen-binding fragment thereof that specifically binds PD-1, an anti-PD-L¹ antibody or antigen-binding fragment thereof that specifically binds PD-L¹, and a combination thereof.
E237. The method of embodiment 236, wherein the anti-PD-L¹ antibody is atezolizumab, avelumab, or durvalumab, wherein the anti-CTLA-4 antibody is tremelimumab or ipilimumab, and wherein the anti-PD-1 antibody is nivolumab or pembrolizumab.
E238. The lipid nanoparticle of any one of embodiments 212-229, and an optional pharmaceutically acceptable carrier, for use in treating or delaying progression of a cancer in an individual, wherein treatment comprises administration of the lipid nanoparticle.
E239. The lipid nanoparticle of any one of embodiments 212-229, and an optional pharmaceutically acceptable carrier, for use in treating or delaying progression of a cancer in an individual, wherein treatment comprises administration of the lipid nanoparticle in combination with a composition comprising an immune checkpoint inhibitory polypeptide, and an optional pharmaceutically acceptable carrier.
E240. The lipid nanoparticle of any one of embodiments 238-239, wherein the cancer is a disseminated cancer or a solid tumor.
E241. Use of a lipid nanoparticle of any one of embodiments 212-229, and an optional pharmaceutically acceptable carrier, in the manufacture of a medicament for treating or delaying progression of a cancer in an individual, wherein the medicament comprises the lipid nanoparticle, and an optional pharmaceutically acceptable carrier, and wherein the treatment comprises administration of the medicament.
E242. Use of a lipid nanoparticle of any one of embodiments 212-229, and an optional pharmaceutically acceptable carrier, in the manufacture of a medicament for treating or delaying progression of a cancer in an individual, wherein the medicament comprises the lipid nanoparticle, and an optional pharmaceutically acceptable carrier, and wherein the treatment comprises administration of the medicament in combination with a composition comprising an immune checkpoint inhibitor polypeptide, and an optional pharmaceutically acceptable carrier.
E243. The use of a lipid nanoparticle according to any one of embodiments 241-242, wherein the cancer is a disseminated cancer or a solid tumor.
E244. A kit comprising a container comprising the lipid nanoparticle of any one of embodiments 212-229, and an optional pharmaceutically acceptable carrier, and a package insert comprising instructions for administration of the lipid nanoparticle for treating or delaying progression of a cancer in an individual.
E245. The kit of embodiment 244, wherein the package insert further comprises instructions for administration of the lipid nanoparticle in combination with a composition comprising an immune checkpoint inhibitor polypeptide, and an optional pharmaceutically acceptable carrier, for treating or delaying progression of a cancer in an individual.
E246. A kit comprising a container comprising the lipid nanoparticle of any one of embodiments 212-229, and an optional pharmaceutically acceptable carrier, and a package insert comprising instructions for administration of the medicament alone, or in combination with a composition comprising an immune checkpoint inhibitor polypeptide, and an optional pharmaceutically acceptable carrier, for treating or delaying progression of a cancer in an individual.
E247. The kit of any one of embodiments 244-246, wherein the cancer is a disseminated cancer or a solid tumor.
E248. A method for enhancing an immune response in a subject, comprising administering to the subject the lipid nanoparticle of any one of embodiments 212-229.
E249. A method for enhancing T cell activation in a subject, comprising administering to the subject the lipid nanoparticle of any one of embodiments 212-229.
E250. A method for enhancing NK cell activation in a subject, comprising administering to the subject the lipid nanoparticle of any one of embodiments 212-229.
E251. The method of any one of embodiments 248-250, wherein the subject has a cancer.
E252. The method of embodiment 251, wherein the cancer is a disseminated cancer or a solid tumor.
E253. The method of any one of embodiments 248-252, further comprising administering an immune checkpoint inhibitor polypeptide.
E254. The lipid nanoparticle of any one of embodiments 212-214 and 218-219, comprising:

(i) an ionizable lipid;

(ii) a sterol or other structural lipid;

(iii) a polynucleotide of any one of claims 1-76;

(iv) optionally, a non-cationic helper lipid or phospholipid; and

(v) optionally, a PEG-lipid.
E255. The lipid nanoparticle of embodiment 254, which comprises a phytosterol or a combination of a phytosterol and cholesterol.
E256. The lipid nanoparticle of embodiment 255, wherein the phytosterol is selected from the group consisting of β-sitosterol, stigmasterol, β-sitostanol, campesterol, brassicasterol, and combinations thereof.
E257. The lipid nanoparticle of embodiment 255, wherein the phytosterol comprises a sitosterol or a salt or an ester thereof.
E258. The lipid nanoparticle of embodiment 255, wherein the phytosterol comprises a stigmasterol or a salt or an ester thereof.
E259. The lipid nanoparticle of embodiment 255, wherein the phytosterol is beta-sitosterol

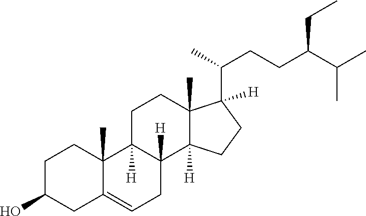

or a salt or an ester thereof.
E260. The lipid nanoparticle of embodiment 254, wherein the phytosterol or a salt or ester thereof is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.
E261. The lipid nanoparticle of embodiment 260, wherein the phytosterol is β-sitosterol.
E262. The lipid nanoparticle of embodiment 260, wherein the phytosterol is β-sitostanol.
E263. The lipid nanoparticle of embodiment 260, wherein the phytosterol is campesterol.
E264. The lipid nanoparticle of embodiment 260, wherein the phytosterol is brassicasterol.
E265. The lipid nanoparticle of any one of embodiments 254-264, wherein the ionizable lipid comprises a compound of any of Formulae (I I), (I IA), (IIB), (I II), (I IIa), (I (I IIc), (I IId), (I lie), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8).
E266. The lipid nanoparticle of any one of embodiments 254-264, wherein the ionizable lipid comprises a compound selected from the group consisting of Compound X, Compound Y, Compound 1-48, Compound I-50, Compound 1-109, Compound I-111, Compound 1-113, Compound 1-181, Compound 1-182, Compound 1-244, Compound 1-292, Compound 1-301, Compound 1-309, Compound 1-317, Compound 1-321, Compound 1-322, Compound 1-326, Compound 1-328, Compound 1-330, Compound 1-331, Compound 1-332, Compound 1-347, Compound 1-348, Compound 1-349, Compound 1-350, Compound 1-352 and Compound I-M.
E267. The lipid nanoparticle of any one of embodiments 254-264, wherein the ionizable lipid comprises a compound selected from the group consisting of Compound X, Compound Y, Compound 1-321, Compound 1-292, Compound 1-326, Compound 1-182, Compound I-301, Compound 1-48, Compound I-50, Compound 1-328, Compound 1-330, Compound I-109, Compound I-111 and Compound I-181.
E268. The lipid nanoparticle of any one of embodiments 254-267, wherein the LNP comprises a phospholipid, and wherein the phospholipid comprises a compound selected from the group consisting of DSPC, DMPE, and Compound H-409.
E269. The lipid nanoparticle of any one of embodiments 254-268, wherein the LNP comprises a PEG-lipid.
E270. The lipid nanoparticle of embodiment 269, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E271. The lipid nanoparticle of embodiment 270, wherein the PEG lipid comprises a compound selected from the group consisting of Compound P-415, Compound P-416, Compound P-417, Compound P-419, Compound P-420, Compound P-423, Compound P-424, Compound P-428, Compound P-L¹, Compound P-L2, Compound P-L3, Compound P-L4, Compound P-L6, Compound P-L8, Compound P-L9, Compound P-L¹⁶, Compound P-L¹⁷, Compound P-L¹⁸, Compound P-L¹⁹, Compound P-L22, Compound P-L23 and Compound P-L25.
E272. The lipid nanoparticle of embodiment 271, wherein the PEG lipid comprises a compound selected from the group consisting of Compound P-428, Compound PL-16, Compound PL-17, Compound PL-18, Compound PL-19, Compound PL-1, and Compound PL-2.
E273. The lipid nanoparticle of any one of embodiments 254-272, which comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid.
E274. The lipid nanoparticle of any one of embodiments 254-272, which comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid.
E275. The lipid nanoparticle of any one of embodiments 254-272, which comprises about 50 mol % ionizable lipid, about 10 mol % non-cationic helper lipid or phospholipid, about 38.5 mol % sterol or other structural lipid, and about 1.5 mol % PEG lipid.
E276. The lipid nanoparticle of any one of embodiments 254-275, wherein the mol % sterol or other structural lipid is 18.5% phytosterol and the total mol % structural lipid is 38.5%.
E277. The lipid nanoparticle of any one of embodiments 254-275, wherein the mol % sterol or other structural lipid is 28.5% phytosterol and the total mol % structural lipid is 38.5%.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: LNP Transfection Efficiency of AML Cell Lines

In this example, transfection conditions and LNP components were studied to optimize transfection efficiency of AML cells in vitro.

In a first series of experiments, AML cells were transfected with mRNAs in LNPs under conditions in the presence or absence of: human serum, mouse serum, human immunoglobulin (IgM or IgG), human PBMCs, or human complement (with or without human serum, human IgM, and/or human IgG). The results (data not shown) demonstrated that addition of human serum, human PBMCs, human complement, or human complement and human immunoglobulin increased transfection efficiency of AML cell lines in vitro. The results suggest that both immunoglobulin recognition and complement deposition on immunoglobulin, followed by complement receptor binding, is required for AML cell uptake of the mRNAs in LNPs.

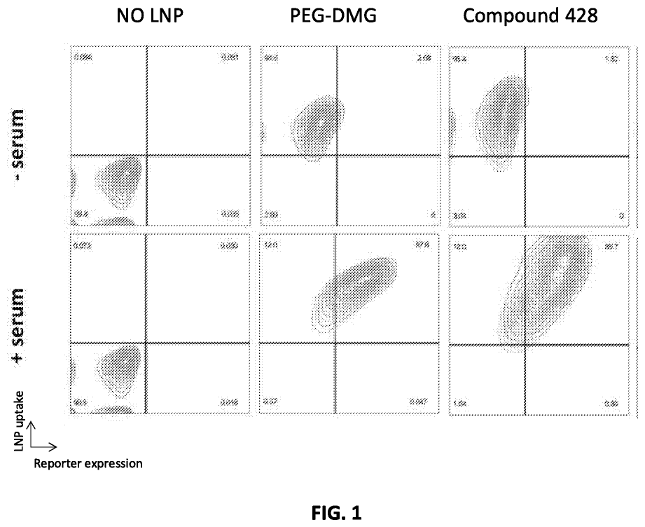

In a second series of experiments, transfection of AML cells in vitro was compared using LNPs containing different PEG-modified lipids. Lipid nanoparticles were formulated comprising: 50% ionizable amino lipid (Compound X), 10% phospholipid; 38.5% cholesterol and 1.5% PEG-modified lipid, wherein the PEG-modified lipid was either 1,2-dimyristoyl-sn-glycerol methoxypolyethylene glycol (PEG-DMG) or Compound 428. Kasumi-1 AML cells were transfected in vitro with: (i) no LNP, (ii) the Compound X/PEG-DMG LNP or (iii) the Compound X/Compound 428 LNP, in the presence or absence of human serum. The results are shown in FIG. 1, which demonstrates efficient transfection of the AML cells with either the Compound X/PEG-DMG LNP or the Compound X/Compound 428 LNP in the presence of human serum.

In a third series of experiments, a series of AML cell lines (ATCC or DSMZ) was tested for transfection efficacy comparing the Compound X/PEG-DMG LNP and the Compound X/Compound 428 LNP. The results are summarized below in Table 17:

TABLE 17
Comparison of Transfection Efficiency
of AML Cells with Different LNPs

| | | PEG DMG | | Compound 428 | |
|---|---|---|---|---|---|
| Cell Line | Nature | −HS* | +HS | −HS | +HS |
| HEL | Erythroleukemia | 90.1 | 99.9 | 89.9 | 99.8 |
| MOLM-13 | Monocyte | 3.9 | 69.7 | 3.5 | 53.7 |
| Kasumi-1 | Myeloblast | 2.88 | 87.6 | 1.52 | 85.7 |
| MV-4-11 | Myelomonocyte | 0 | 65.5 | 1.2 | 60.8 |
| NOMO-1 | Myeloblast | 54.8 | 99.3 | 4 | 35 |
| OCl-AML5 | Monocyte | 0 | 1.71 | 0 | 0.4 |
| MOLM-16 | Megakaryoblast | 99.7 | 99.9 | 90 | 99 |
| KG-1 | Macrophage | 2.1 | 6.1 | 0.3 | 6.6 |
| HL-60 | Promyeloblast | 0 | 22.7 | 0.1 | 2.3 |
| GDM-1 | Monoblast | 0.4 | 3.1 | 0.1 | 13 |
| EOL-1 | Eosinophil | 0.04 | 7.9 | 0.043 | 1.6 |

*HS = human serum

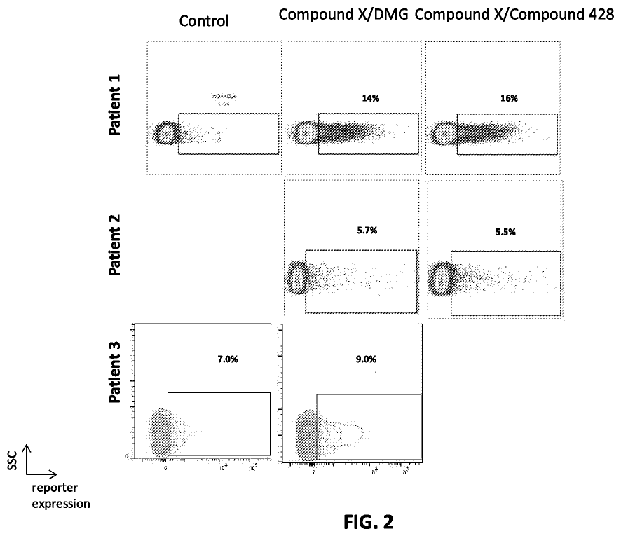

In a further experiment, transfection efficiency of LNP in primary AML cells was assessed. Specifically, 8 different primary AML samples were transfected with Compound X/PEG-DMG LNP or Compound X/Compound 428 LNP after the LNPs were opsonized in human serum. The results are shown in FIG. 2, which demonstrates efficient transfection of the AML cells with either the Compound X/PEG-DMG LNP or the Compound X/Compound 428 LNP.

The results demonstrate that the Compound X/PEG-DMG LNP and the Compound X/Compound 428 LNP exhibited comparable transfection efficiencies in the different cell lines tested.

### Example 2: Inhibition of Tumor Growth by Combination Therapy with OX40L, IL-12 and IL-15 mRNAs

In this example, a murine tumor model of Acute Myeloid Leukemia (AML) was used to examine the efficacy of combination therapy using mRNAs encoding mOX40L, mIL-12 and hIL-15/IL-15Rα in solid tumors and disseminated cancers.

AML tumors were established subcutaneously in DBA/2 mice. Mouse AML cells P388D1 (ATCC No. CCL-46; ATCC, Manassas, Va.) were cultured according to the vendor's instructions. Cells were inoculated subcutaneously in mice to generate subcutaneous tumors. Tumors were monitored for size and palpability. Given the cellular origin of this line and its growth characteristics in mice, P388D1 cells serve as a model for both disseminated cancers and solid tumors.

Once the tumors were established, animals were separated into three groups. Group 1 was treated with a control mRNA NST (12.5 μg), Group 2 was treated with an mRNA encoding mOX40L (5 μg) and an mRNA encoding hIL-15 linked to the sushi domain of hIL-15Rα (hIL-15 RLI) (5 μg) and Group 3 was treated with an mRNA encoding mOX40L (5 μg), an mRNA encoding hIL-15 RLI (5 μg), and an mRNA encoding mIL-12 (2.5 μg). The mRNA encoding mOX40L encodes an amino acid sequence as set forth in SEQ ID NO: 3. The mRNA encoding IL-15 RLI comprises an open reading frame comprising SEQ ID NO: 32, which encodes the amino acid sequence as set forth in SEQ ID NO: 31. For Group 2, the control mRNA NST was also included to achieve a total mRNA dose of 12.5 μg per animal. mRNAs were formulated in separate lipid nanoparticles comprising: 50% ionizable amino lipid (Compound X), 10% phospholipid; 38.5% cholesterol and 1.5% PEG lipid. Intratumoral dosing for each group was every 7 days (Q7D), beginning 7 days after tumor implantation. Groups 1 and 2 were treated with three doses. Group 3 was treated with a single dose, since some toxicity was observed with the dose level used.

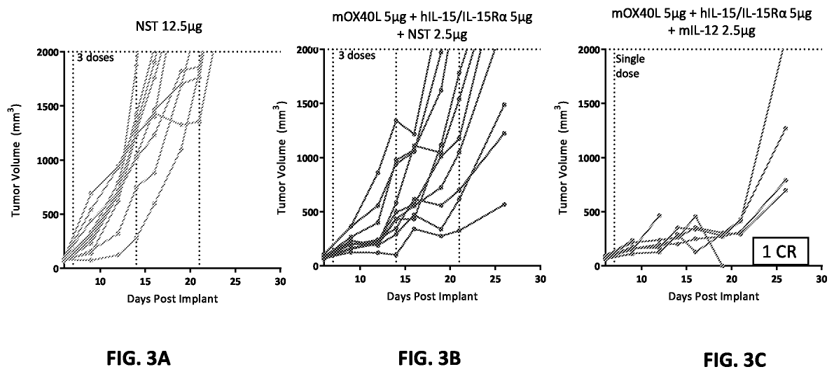

Results are shown in the graphs of FIG. 3A (Group 1), FIG. 3B (Group 2) and FIG. 3C (Group 3), which show tumor volume over time.

The results demonstrate that, compared to treatment with mOX40L alone, doublet combination therapy with mOX40L and hIL-15 RLI led to slower tumor growth, whereas triplet combination therapy with mOX40L, hIL-15 RLI and mIL-12 led to significant inhibition of tumor growth, with one complete remission observed. These results demonstrate the efficacy of the mRNA combination therapy in cancer.

### Example 3: In vitro Expression and Bioactivity of Cell-Associated and Tethered IL-15 Constructs

To assess the potential for enhancing anti-tumor efficacy in cancer, mRNA(s) encoding cell-associated IL-15 constructs were generated capable of trans-presenting IL-15.

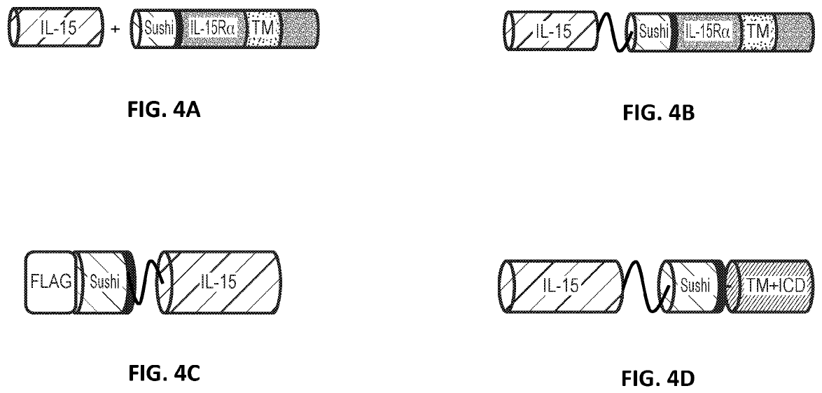

Specifically, mRNAs comprising nucleotide sequences encoding an IL-15 polypeptide and a miRNA binding site (miR-122) in the 3′UTR were prepared. The sequences were: hIL-15+hIL-15Rα (cell-associated IL-15; SEQ ID NOs: 17 and 13, respectively), encoding human IL-15 and IL-15Rα, which comprises a sushi domain and transmembrane domain (FIG. 4A); hIL-15_IL-15Rα (tethered IL-15; SEQ ID NO: 27), encoding human IL-15 linked to full length human IL-15Rα (FIG. 4B), and hIL-15 RLI (SEQ ID NO: 31), encoding chimera of IL-15Rα sushi domain linked to IL-15 via a linker (FIG. 4C), hIL-15_CD80TID (tethered IL-15; SEQ ID NO: 123), encoding human IL-15 linked to a sushi domain, and a transmembrane domain and intracellular domain derived from CD80 (FIG. 4D). The mRNA open reading frame sequences are shown in SEQ ID NOs: 122, 22, 28, 32 and 124, respectively. Each mRNA comprised a 5′ UTR having the sequence set forth in SEQ ID NO: 117 or SEQ ID NO: 12, and 3′UTR having the sequence set forth in SEQ ID NO: 121 or SEQ ID NO: 77.

The cell-associated IL-15 construct comprises two separate mRNAs encoding the separate components, wherein the IL-15 binds to the IL-15Rα sushi domain with high affinity, thereby restricting IL-15 to IL-15Rα expressing cells. The tethered IL-15 creates a fusion protein with the IL-15 cytokine linked to IL-15Rα which is naturally expressed on the cell membrane, thereby tethering IL-15 to the cell it is expressed in.

HeLa cells were seeded on 6-well plates (BD Biosciences, San Jose, USA) one day prior to transfection. Next, mRNAs comprising hIL-15+hIL-15Rα; hIL-15_IL-15Rα; hIL-15 RLI; or hIL-15_CD80TID were individually transfected into the HeLa cells using 2 μg mRNA and 4 μL Lipofectamine 2000 in 150 μL Opti-MEM per well and incubated. HeLa cells exposed to transfection reagent with no mRNA served as a negative control (i.e., “Mock”). Transfection media was removed after 4 hours and replaced with fresh growth medium for the remainder of the incubation period. After 24 hours, supernatant was collected from each well, and the cells in each well were lysed using a consistent amount of lysis buffer. The amount of IL-15 (ng/well) in the supernatant and lysate for each well was then quantified by a standard ELISA assay.

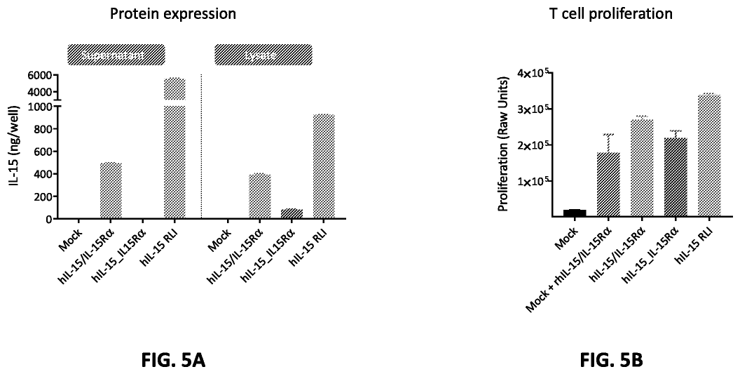

FIG. 5A shows that tethered IL-15 (hIL-15_IL-15Rα) was only expressed in the lysate, and not the supernatant, indicating successful tethering to the cells. In contrast, separate mRNAs encoding hIL-15 and hIL-15Rα, along with hIL-15 RLI, resulted in IL-15 expression in both the supernatant and the lysate.

To assess bioactivity, 25,000 CTLL-2 cells were cultured with a fixed number of Mitomycin C-treated HeLa cells from a HeLa cell culture transfected with one of the noted constructs and further including a fixed dilution of supernatant from the HeLa cell culture. Recombinant human IL-15 and human IL-15Rα (rhIL-15/IL-15Rα), mixed together at a 1:1 molar ratio, were also added to a subset of negative control cultures (“Mock+rhIL-15/IL-15Rα”). After 72 hours of culture, the proliferation of CTLL-2 cells in each culture was determined using the CellTiter-Glo® Luminescent Cell Viability Assay kit (Promega Corporation, Madison, Wis.) according to the manufacturer's instructions.

FIG. 5B shows all the IL-15 constructs tested induce T cell proliferation, indicating bioactivity for each construct.

An additional experiment was done to test the expression of the IL-15 constructs using different mRNA versions encoding the same construct. The methods were the same as those described above. Specifically, lysate and supernatant were collected 24 hours post-transfection of HeLa cells with Lipofectamine Expression was measured using the IL-15/IL-15Rα complex ELISA kit from R&D Systems (DY6924). hIL-15_IL-15Rα having a tPA6 signal peptide (“tPA6 IL15-IL-15Rα”) was encoded by SEQ ID NOs: 28, 29 (var2) or 30 (var3); hIL-15_IL-15Rα having an IgE signal peptide (“IgE IL15-IL15Rα”) was encoded by SEQ ID NOs: 24, 25 (var2) or 26 (var3); and hIL-15_CD80TID (“tPA6 ILR linker 80TID”) was encoded by SEQ ID NOs: 124, 125 (var2) or 126 (var3). Separate mRNAs encoding hIL-15 and hIL-15Rα (Hs IL15_mod/Hs IL15Rα mod1). FIG. 5C shows protein expression of IL-15 in the supernatant and the lysate in cells transfected with the various mRNAs. FIG. 5D shows the percent of protein shed, which was calculated as supernatant expression/lysate expression+supernatant expression. These results indicated low expression in the supernatant is not solely due to low expression overall, but that the lower percentage of total protein that is translated ends up in the supernatant. Moreover, the results show almost no expression in the supernant in cells transfected with the hIL-15 linked to the transmembrane and intracellular domains of CD80.

### Example 4: Further Characterization of AML Treatment with Tethered and Cell-Associated Cytokine mRNAs

In this example, a C1498 tumor model was used to further examine the efficacy of treatment with tethered and cell-associated cytokine mRNA constructs. The C1498 tumor model was used to determine efficacy in both disseminated cancers and solid tumors, as these AML cells are used to form subcutaneous tumors (i.e. solid tumors). In addition to utilizing mRNA encoding the cell-associated human IL-15 construct described in Example 3, mRNA encoding tethered murine IL-12 was also utilized. Specifically, mRNA encoding a murine IL-12 polypeptide connected by a linker to a mouse PGFRB transmembrane domain (mIL-12PTM) was prepared. The amino acid sequence is provided in SEQ ID NO: 45, and the open reading frame is provided in SEQ ID NO: 44.

C1498 syngeneic subcutaneous tumors were established in C57Bl/6 mice and the mice were treated intratumorally with single (mOX40L or cell-associated hIL-15/IL-15Rα or mIL-12TM), doublet (mOX40L+cell-associated hIL-15/IL-15Rα) or triplet mRNA (mOX40L+cell-associated hIL-15/IL-15Rα+mIL-12TM) constructs in LNPs. The LNPs were formulated comprising: 50% ionizable amino lipid (Compound X), 10% phospholipid; 38.5% cholesterol and 1.5% PEG-modified lipid, wherein the PEG-modified lipid was 1,2-dimyristoyl-sn-glycerol methoxypolyethylene glycol (PEG-DMG). Mice were treated with a total of 12.5 μg mRNA in LNPs. The mOX40L and hIL-15/IL-15Rα constructs were used at 5 μg mRNA each and the mIL-12TM construct was used at 5 μg mRNA as a single agent and at 2.5 μg mRNA when used in combinations, with total mRNA being brought to 12.5 μg using the NST control mRNA as necessary.

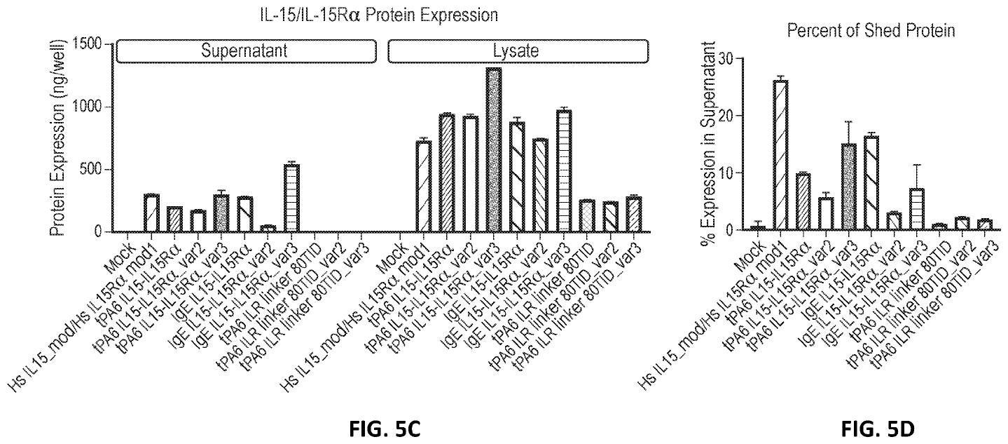

The results provided in FIG. 6A-6F show tumor growth as measured by tumor volume over 65 days in the mice treated with either the single agents (FIGS. 6B, 6C and 6D), the doublet mRNA combination (FIG. 6E) or the triplet mRNA combination (FIG. 6F). The results demonstrate that the triplet combination was highly efficacious in the C1498 subcutaneous tumor model, with 6 complete responses (CR) observed.

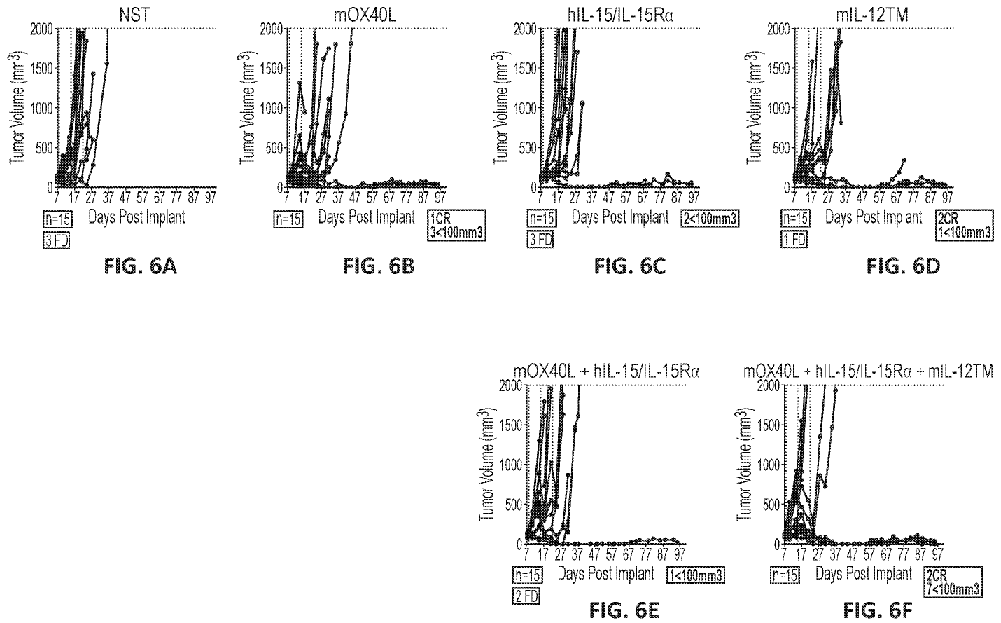

The results provided in FIGS. 7A-7C show percent survival of the mice treated with the various single agents (FIG. 7A), the various mOX40L+hIL-15/IL-15Rα doublets (FIG. 7B) and the various mOX40L+hIL-15/IL-15Rα+mIL-12TM triplets (FIG. 7C). The results demonstrated that the mOX40L+hIL-15/IL-15Rα+mIL-12TM triplet combination using tethered cytokines significantly improved the survival of the mice in the C1498 subcutaneous tumor model.

A repeat experiment demonstrated similar tumor growth inhibition, showing that the efficacy of the triplet intratumoral treatment was reproducible. The results from the second study (n=15) and the combined results of the two studies (n=30) are summarized in Table 18, which shows tumor growth as measured by tumor volume over 65 days in mice treated with either a control, the singlet or triplet mRNA combination:

TABLE 18
Efficacy of combination mRNA in C1498 tumor model

| mRNA | combined CR | % CR (n = 30) |
|---|---|---|
| NST | 0 | 0 |
| mOX40L | 5/30 | 17 |
| hIL-15/15Rα | 4/30 | 13 |
| mIL-12TM | 5/30 | 17 |
| mOX40L + hIL-15/IL-15Rα + | 14/30 | 47 |
| mIL-12TM | | |

The combination of mOX40L+hIL-15/IL-15Rα+mIL-12TM exhibited a complete response (CR) rate of 47%, whereas the CR rate for the single agents was β-17%, demonstrating that the triplet combination was more efficacious when administered intratumorally than any single agent. Moreover, rechallenge of all responders led to no regrowth suggesting durable anti-tumor immune response.

Additionally, the efficacy of a single dose of treatment with the mOX40L+hIL-15/IL-15Rα+mIL-12TM triplet versus multiple doses of treatment (Q7D×3) with the triplet was compared in a total of four independent studies (n=59 total). In addition, the single or multiple dose regimen of the triplet was compared to the doublet combinations of mOX40L+mIL-12TM and mIL-12TM+hIL-15/IL-15Rα. The results of a representative study (n=15) are summarized in Table 19 (as compared to the NST control) and the results from the four intratumoral studies for the triplet combination are also summarized (Table 19):

TABLE 19
Efficacy of single- and multi-dose combination mRNA

| | | | combined CR |
|---|---|---|---|
| | Dosing | | from 4 studies, |
| mRNA | schedule | CR | n = 59 (% CR) |
| NST | Single- and | 0 | NA |
| | multi-doses | | |
| hIL-15/IL-15Rα + mIL-12TM | Q7Dx3 | 5/15 | NA |
| mOX40L + mIL-12TM | Q7Dx3 | 8/15 | NA |
| mOX40L + hIL-15/IL-15Rα + | single | 4/15 | 24 (41%) |
| mIL-12TM | Q7Dx3 | 7/15 | 31 (53%) |

The results demonstrated that both the single dose and multiple dose intratumoral treatment regimens were effective in inhibiting tumor growth, with no significant advantage of multiple dosing being observed. The results also indicated efficacy of the doublet combinations, with the combination of mOX40L+mIL-12TM being similarly effective as multiple doses of the triplet combination in this model.

Overall, these results indicate the combination of a tethered or cell-associated cytokine with a costimulatory molecule (e.g., OX40L) provide anti-tumor efficacy in cancer, including disseminated cancers and solid tumors.

### Example 5: Combined Treatment of AML with Tethered Cytokine mRNAs and Immune Checkpoint Inhibitors

In this example, the C1498 tumor model described in Example 4 was used to examine the efficacy of treatment with tethered and cell-associated cytokine mRNA constructs in combination with immune checkpoint inhibitor agents. Mice were treated intratumorally with the LNP-encapsulated triplet mOX40L+hIL-15/IL-15Rα+mIL-12TM mRNA construct alone or in combination with treatment with either anti-mCTLA-4 antibody, anti-mPD-L¹ antibody or anti-mPD-1 antibody, from BioXCell. Single dose and multiple dose treatment regimens for the triplet mRNA were examined. For multiple dosing, the mRNA construct was administered weekly for two weeks (Q7Dx2) or three weeks (Q7Dx3). The NST mRNA construct, alone or in combination with an immune checkpoint inhibitor, was used as the negative control.

In a first series of experiments, mice (n=12) were treated with a single dose of the mOX40L+hIL-15/IL-15Rα+mIL-12TM triplet mRNA combination (12.5 μg, intratumorally) in combination with anti-mCTLA-4 antibody treatment (10 mg/kg; twice per week for two weeks for a total of four doses). The results are shown in Table 20.

TABLE 20
Efficacy of combination with anti-mCTLA-4 antibody

| | αmCTAL-4 | |
|---|---|---|
| mRNA (single dose) | (10 mpk; BIWx2) | CR |
| NST | − | 0/12 |
| | + | 0/12 |
| mOX40L + hIL-15/15Rα + | − | 6/12 |
| mIL-12TM | + | 9/12 |

The results demonstrated that addition of the anti-mCTLA-4 immune checkpoint inhibitor led to an improvement in the complete response (CR) rate for the single dose of the mOX40L+hIL-15/IL-15Rα+mIL-12TM triplet mRNA construct. More specifically, for intratumoral treatment with the single dose of the triplet alone, a 50% CR rate (6/12) was observed, whereas the combination of the single dose of the triplet with anti-mCTLA-4 treatment led to a 75% CR rate (9/12).

In a second series of experiments, mice (n=15) were treated with either a single dose or multiple doses (once per week for three weeks for a total of three doses) of the mOX40L+hIL-15/IL-15Rα+mIL-12TM triplet mRNA combination (12.5 μg, intratumorally) in combination with anti-mPD-L¹ antibody treatment (10 mg/kg; twice per week for two weeks for a total of four doses). For the single dose experiments, no significant improvement in CR was observed with addition of anti-mPD-L¹ treatment as compared to treatment with the triplet mRNA alone (data not shown). The results for the multiple dose triplet mRNA treatment are shown in Table 21:

TABLE 21
efficacy of combination mRNA with immune checkpoint inhibitors

| | | | Checkpoint | | |
|---|---|---|---|---|---|
| | | Dosing | inhibitor | CR | |
| | mRNA | schedule | (10 mpk) | (% CR) | |
| Expt. 2 | NST | multi-dose | — | 0 | |
| | | Q7Dx3 | | | |
| | | | αmPD-L1 | 1/15 | (7) |
| | mOX40L + hIL-15/ | Q7Dx3 | — | 6/15 | (40) |
| | 15Rα + | | αmPD-L1 | 12/15 | (80) |
| | mIL-12TM | | | | |
| Expt. 3 | NST | Q7Dx3 | — | 0 | |
| | | Q7Dx2 | αmPD-L1 | 1/15 | (7) |
| | | | αmPD-L1 | 1/15 | (7) |
| | mOX40L + hIL-15/ | Q7Dx2 | — | 5/15 | (33) |
| | 15Rα + | | αmPD-L1 | 9/15 | (60) |
| | mIL-12TM | | αmPD-L1 | 14/15 | (93) |

The results demonstrated that addition of the anti-mPD-L¹ immune checkpoint inhibitor led to an improvement in the complete response (CR) rate for the multiple dose regimen of the mOX40L+hIL-15/IL-15Rα+m IL-12TM triplet mRNA construct. More specifically, for intratumoral treatment with the multiple dose regimen of the triplet alone, a 40% CR rate (6/15) was observed, whereas the combination of the multiple doses of the triplet with anti-mPD-L¹ treatment led to an 80% CR rate (12/15).

In a third series of experiments, mice (n=15) were treated with multiple doses (once per week for two weeks for a total of two doses) of the mOX40L+hIL-15/IL-15Rα+mIL-12TM triplet mRNA construct (12.5 μg, intratumorally) in combination with anti-mPD-L¹ antibody treatment (10 mg/kg; twice per week for two weeks for a total of four doses) or anti-mPD-1 antibody treatment (10 mg/kg; twice per week for two weeks for a total of four doses). The results are shown in Table 21, which demonstrate the addition of the anti-mPD-L¹ or anti-mPD-1 immune checkpoint inhibitor leads to an improvement in the complete response (CR) rate of the mOX40L+hIL-15/IL-15Rα+mIL-12TM triplet mRNA construct. More specifically, for intratumoral treatment with the multiple dose regimen of the triplet alone, a 33% CR rate (5/15) was observed, whereas the combination of the triplet with anti-mPD-L¹ treatment led to a 60% CR rate (9/15), and combination with anti-mPD-1 treatment led to a 93% CR rate (14/15).

These experiments confirmed the anti-tumor effectiveness of the triplet mRNA construct alone using either a single or multiple doses intratumorally and demonstrated that combined treatment with an immune checkpoint inhibitor can further improve the effectiveness of the therapy.

### Example 6: Characterization of Disseminated AML Model and Treatment with mRNAs

To further assess the efficacy of the combination of mRNA constructs described above in treating AML, a disseminated model of AML was generated to recapitulate the physiological characteristics of AML disease in humans. The AML cells expressed GFP and luciferase, which allows for monitoring of leukemic burden by bioluminescence imaging (BLI) and flow cytometry measurement of GFP+ cells in the blood. Such AML cells were implanted intravenously via tail vein injection in C₅₇BL/6 mice. BLI signal was evident in the long bones and spleens 5 days after implantation, whereas GFP+ cells were detectable by flow cytometry in the circulating blood 15 days after implantation (data not shown). Thus, this model recapitulates the human disease by propagating in various hematopoietic tissues over the course of disease progression.

mRNAs encoding OX40L, mIL-12TM (tethered IL-12 construct comprising murine IL-12 linked to a murine PDGFR transmembrane domain), and cell-associated hIL-15 (separate mRNAs encoding human IL-15 and human IL-15Rα), were prepared as described above. The mRNAs were formulated in separate LNPs having a molar ratio of 50% ionizable amino lipid (Compound X), 10% phospholipid; 38.5% cholesterol and 1.5% PEG-modified lipid, wherein the PEG-modified lipid was Compound 428.

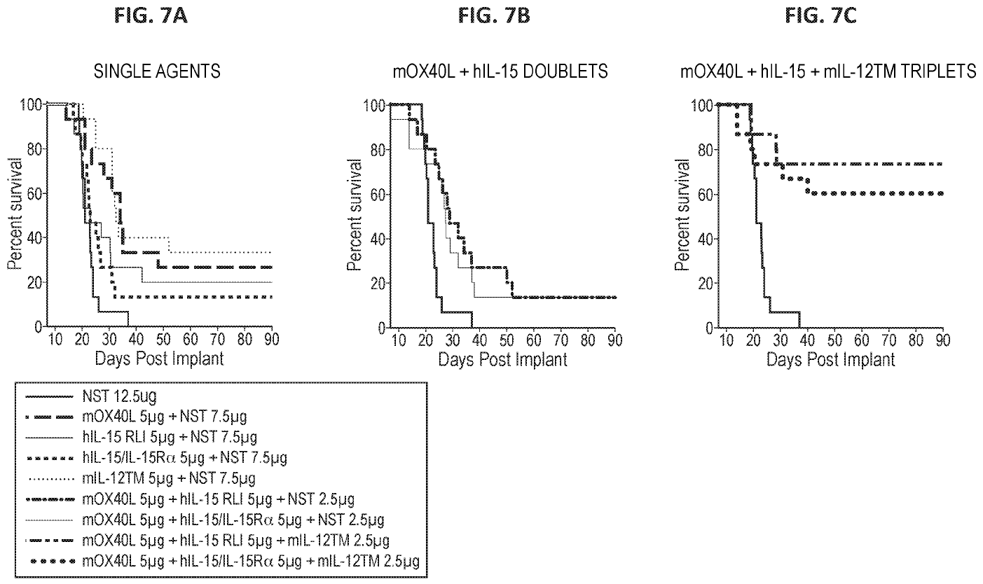

Mice were administered Tris/sucrose as a control, or 2 mg/kg (total mRNA) of mRNAs encoding mOX40L, mIL-12TM, hIL-15, and hIL-15Rα, administered intravenously at day 9 after implantation. Light output was measured by BLI over 90 days by administering luciferin to mice intraperitoneally 5-10 minutes prior to imaging mice on an IVIS in vivo imaging system (Perkin Elmer). FIG. 8A shows 3/5 mice had no measurable AML disease when administered mRNAs encoding mOX40L, tethered mIL-12 and cell-associated hIL-15. When these mice were re-challenged with the AML cells, 3/3 mice had no measurable BLI signal at day 70 (data not shown). Further, blood was collected at day 21 after re-challenge, and the number of GFP+ cells per microliter of blood was measured via flow cytometry by detecting native GFP fluorescence from AML cells in tissue samples that were co-stained for mouse CD45 and subsequently analyzed on a BD LSRFortessa flow cytometer. FIG. 8B shows mice that received mRNAs encoding mOX40L, tethered mIL-12 and cell-associated hIL-15 had reduced GFP+ cells relative to mice that received Tris/sucrose.

A separate study was conducted to assess changes in efficacy based on dosing regimens, as it was previously found that although a single dose of 2 mg/kg of mRNA resulted in efficacy, toxicity events were observed with repeated weekly doses at this dose level in this model. Accordingly, mice were implanted with mouse AML cells, and those with BLI signal of 5×10⁵ to 5×10⁶ photons per second 7 days after implantation were dosed intravenously at day 9 with LNPs encapsulating mRNAs encoding mOX40L, tethered mIL-12 and cell-associated hIL-15, as described supra. Mice either received 2 mg/kg total mRNA once a week for three weeks (Q7D×3), a total of 2 mg/kg mRNA over three weeks (“fractionated dose”), i.e., 0.22 mg/kg three times a week for three weeks (TIW×3), i.e., 0.67 mg/kg a week, or 0.67 mg/kg once a week for three weeks. Table 22 show BLI measurements up to 49 days post-implant, wherein the fractionated low doses of mRNA induced efficacy superior to the same dose delivered as a weekly bolus, and equivalent to the high bolus dose group that delivers three times the total amount of mRNA.

TABLE 22
Efficacy of fractionated doses of combination mRNA

| | mRNA dose | Dosing | CR | |
|---|---|---|---|---|
| mRNA | (mg/kg) | schedule | (% CR) | |
| NST | 2 | QDx1 | 0 | |
| mOX40L + hIL-15/ | 2 | Q7Dx3 | 7/12 | (58) |
| IL-15Rα + | 0.67 | Q7Dx3 | 1/12 | (8) |
| mIL-12TM | 0.22 | TIWx3 | 5/12 | (42) |

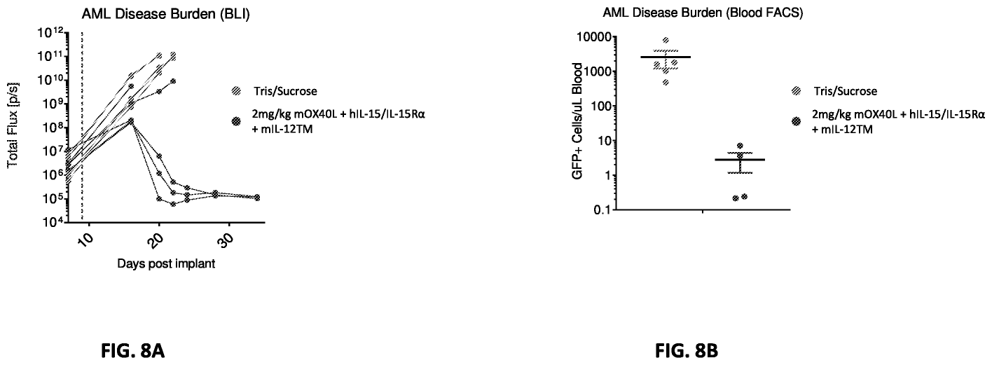

In addition, the number of GFP+ cells in the blood of the same mice was determined as described above. FIG. 9 shows the total number of GFP+ cells per microliter of blood (left), and the percentage of GFP+CD45+ cells (right). Both dosing regimens resulted in similar levels of GFP+ cells in the blood, which was lower compared to mice that received control.

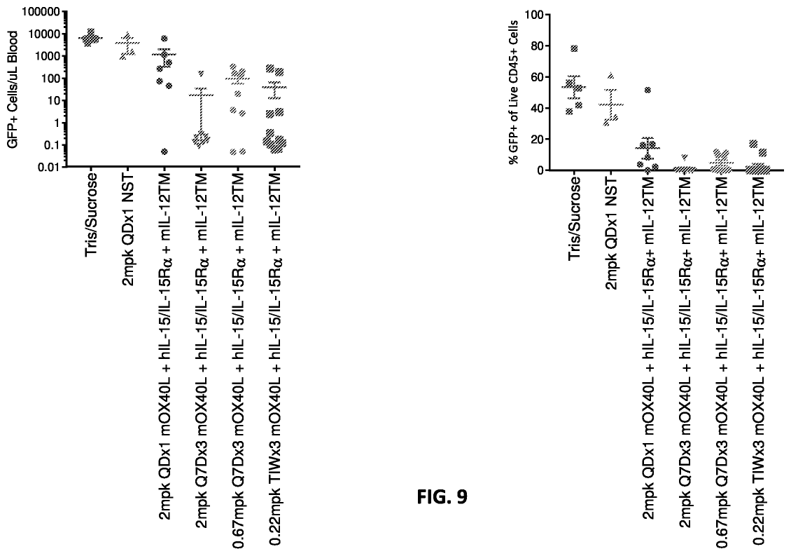

Survival of the mice that received various doses was also determined. In addition to the above dosing regimens, a group of mice received once dose of 2 mg/kg of the combination of mRNAs. FIG. 10 provides the results, showing higher survival in mice that received 2 mg/kg once a week for three weeks or 0.22 mg/kg three times a week for three weeks.

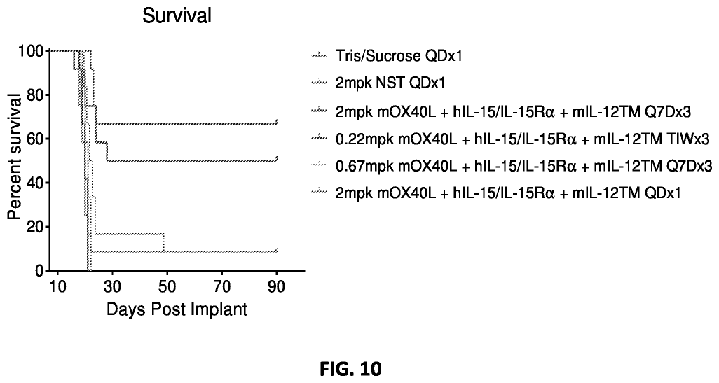

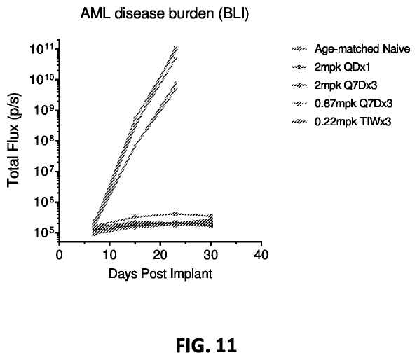

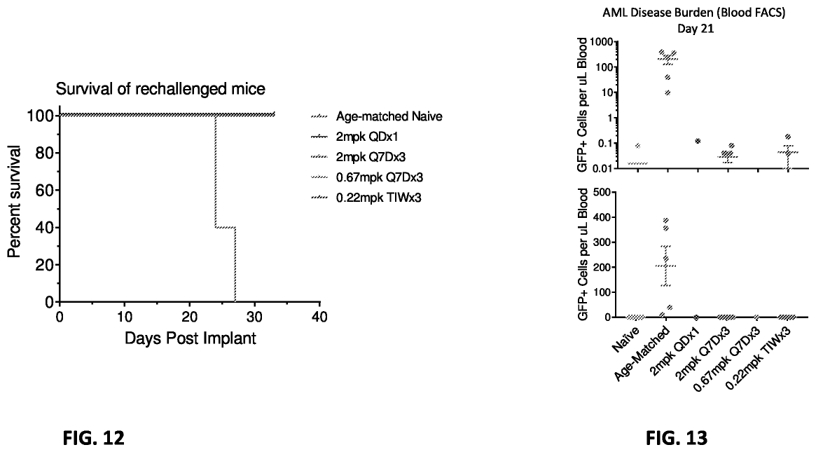

To determine whether the combination of mRNAs encoding mOX40L, tethered mIL-12 and cell-associated hIL-15 provided protective immunity, mice that completely responded to the combination treatment were re-challenged with murine AML cells via tail vein injection. Protective immunity is critical for developing effective immunotherapies, as a durable memory response prevents relapse. As shown in FIG. 11, all the re-challenged complete responders were leukemia-free 70 days post re-challenge. FIGS. 12 and 13 show that all re-challenged mice survived and had no disease burden as measured by the number of GFP+ cells in the blood, respectively.

These results indicate the combination of mRNAs encoding OX40L, tethered IL-12 and cell-associated IL-15 provides protective immunity, an important feature for cancer immunotherapy. Importantly, this efficacy was observed with intravenous administration of the mRNAs.

### Example 7: Further Characterization of Disseminated AML Model and Treatment with Fractionated Doses of mRNAs

The efficacy of mRNAs encoding mOX40L, tethered mIL-12 and cell-associated hIL-15 in the disseminated AML model was unexpectedly found to vary based on dosing regimen as described in Example 6. To further evaluate this finding, biodistribution of the mRNAs was assessed in two different LNP formulations.

Specifically, 15 days after mice were implanted with murine AML cells, mice were dosed with mRNA encoding mOX40L formulated in LNPs described in Example 6 (LNP1) or LNPs comprising Compound X/cholesterol/beta-sitosterol/PEG-DMG at a ratio of 50:10:10:28.5:1.5 (LNP2). Such lipid nanoparticles (LNPs), which contain beta-sitosterol are described further in PCT Application No. PCT/US19/15913, filed Jan. 30, 2019, the entire contents of which is expressly incorporated herein by reference.

mRNA was administered at single doses of 2 mg/kg, 0.67 mg/kg, or 0.22 mk/kg, and mOX40L expression was analyzed by flow cytometry 24 hours after administration. Alternatively, mice received 0.22 mg/kg three times a week for one week (TIW), and mOX40L expression was determined 24 hours after the third dose.

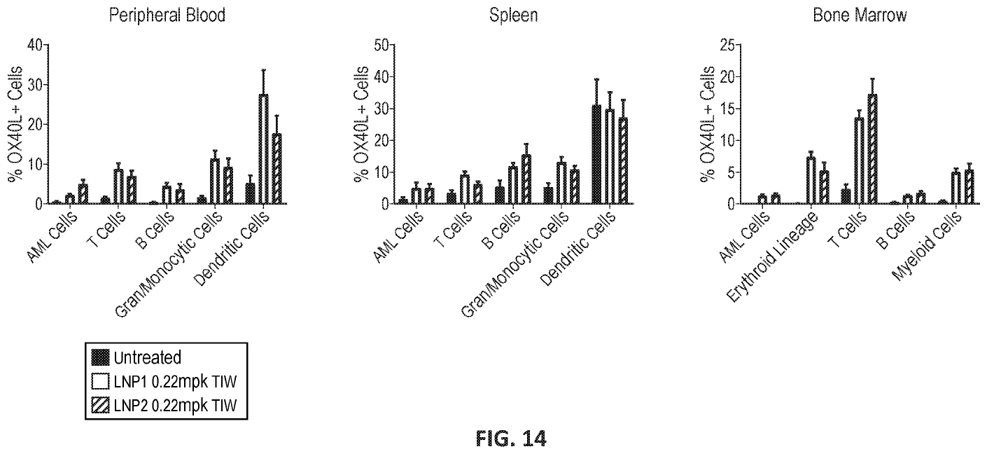

Cells were collected from the peripheral blood, spleen and bone marrow for expression analysis. Individual cell types, i.e., GFP+ AML cells, CD3+ T cells, CD19+ B cells, CD11b+ granulocytic/monocytic cells, CD11c+ dendritic cells, other CD11b+ CD11c+ myeloid cells, and Ter119+ erythroid lineage cells, were evaluated for mOX40L expression. FIG. 14 provides the results for both LNPs administered at 0.22 mg/kg TIW, which indicate no obvious benefit to fractionated doses compared to bolus doses in terms of number of transfected cells or amount of protein produced, which was consistent for all dosing regimens tested (data not shown). AML cells as well as a variety of normal hematopoietic cells are transfected in vivo. Dendritic cells were the best transfected cell type in all the hematopoietic tissues evaluated.

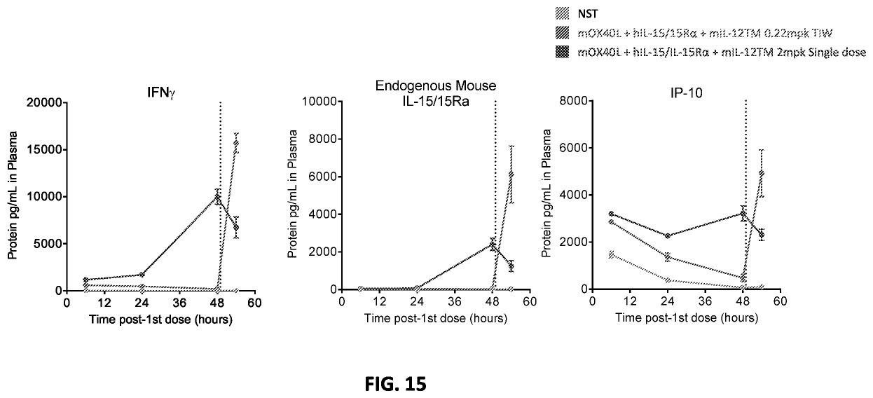

To further assess the efficacy of fractionated dosing, serum cytokine levels were evaluated in mice bearing disseminated AML cells. Mice received either a 2 mg/kg bolus of mRNAs encoding mOX40L, tethered mIL-12 and cell-associated hIL-15, or 0.22 mg/kg of the combination three times a week for one week. Serum was collected over 54 hours and evaluated for expression of mouse IFNγ, endogenous mouse IL-15/IL-15R, and mouse IP-10 at several time points. using Luminex bead-based multi-analyte immunoassay (ThermoFisher Scientific, Waltham, Mass.). FIG. 15 provides the results, which indicate the bolus dose induced higher cytokine levels after the first dose, corresponding to the higher mRNA dose delivered; however, after the second fractionated dose, cytokine levels reach a peak at 6 hours post-dose even higher than that achieved with the higher bolus dose.

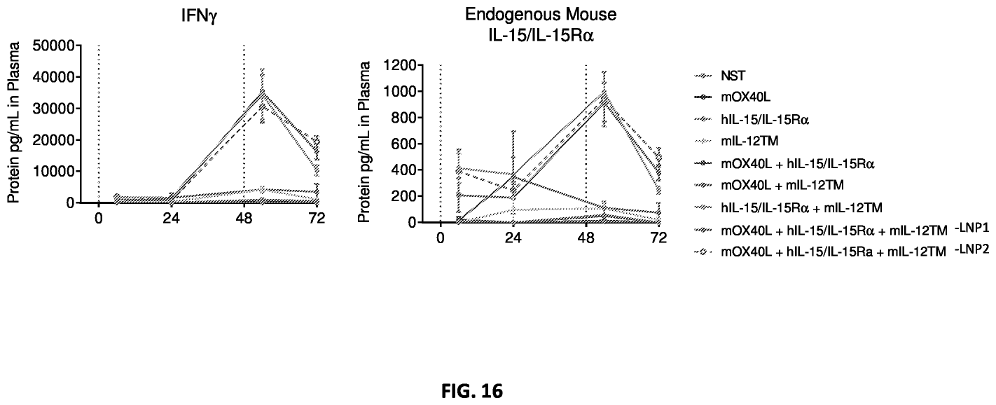

An additional study was conducted to evaluate serum cytokine levels in mice that received single mRNAs or combinations of mRNAs encoding mOX40L, tethered mIL-12 and/or cell-associated hIL-15. mRNAs encapsulated in LNP1 or LNP2 as described above were administered three times a week, and serum collected for analysis of mouse IFNγ and mouse IL-15/IL-15R at 6 and 24 hours after each dose. FIG. 16 provides the results, which indicate a spike in serum levels with both the triplet combination of mRNAs encoding mOX40L, tethered mIL-12 and cell-associated hIL-15, regardless of LNP formulation, and with the doublet combination encoding tethered mIL-12 and cell-associated hIL-15. This result was confirmed in additional studies (data not shown). Cytokines were also induced with other combinations of these three components, but the higher spike in serum cytokines was only observed in treatments including both tethered mIL-12 and cell-associated hIL-15.

In a separate study, the efficacy of mRNAs encoding mOX40L, tethered mIL-12 and cell-associated hIL-15 administered at the same dose for different lengths of time was evaluated. Specifically, mice received 0.22 mg/kg three times a week for either one, two or three weeks, or twice a week for three weeks, of mRNAs encoding mOX40L, tethered mIL-12 and cell-associated hIL-15 formulated in separate LNPs (LNP1 or LNP2). Table 23 shows the efficacy of the combination of these mRNAs was similar when dosed for two or three weeks, but notably lower if only dosed for one week. Extending the dosing schedule beyond one week improves the complete responder rate from 40% to 70-80%. Similar efficacy was shown between dosing twice a week or three times a week for three weeks.

TABLE 23
Efficacy of combination mRNA at different dosing schedules

| | Dose | Dosing | |
|---|---|---|---|
| mRNA | (mg/kg) | schedule | CR |
| NST | 0.22 | TIWx3 | 0 |
| mOX40L + hIL-15/IL-15Rα + | | TIWx1 | 6/10 |
| mIL-12TM (LNP1) | | TIWx2 | 7/10 |
| | | TIWx3 | 8/10 |
| | | BIWx3 | 8/10 |
| mOX40L + hIL-15/IL-15Rα + | 0.22 | TIWx3 | 10/10 |
| mIL-12TM (LNP2) | | | |

### Example 8: Characterization of Efficacy of mRNA Combination Treatment in Disseminated AML Model

The enhanced anti-leukemic efficacy of the combination of mRNAs encoding mOX40L, tethered mIL-12, and cell-associated hIL-15, especially with fractionated dosing, is described in the Examples above. To further evaluate this efficacy, monotherapies and double combinations administered at fractionated doses were assessed.

Specifically, mRNAs encoding mOX40L, cell-associated hIL-15 and tethered mIL-12 were formulated in LNPs as described in Example 6, and administered on day 9 after implantation of murine disseminated AML cells as single agents or in combination to mice having BLI measurements of 5×10⁵ to 5×10⁶ photons per second as of day 7. Mice received 0.22 mg/kg mRNA (0.22 mpk) three times a week for three weeks (TIW×3). Table 24 shows the disease burden as determined by BLI. mOX40L and tethered mIL-12 reduced disease burden as single agents, whereas cell-associated hIL-15 did not relative to control. The addition of mRNA encoding mOX40L to mRNAs encoding tethered mIL-12 or cell-associated hIL-15 enhanced anti-tumor efficacy, whereas the combination of tethered mIL-12 and cell-associated hIL-15 provided minimal efficacy. Notably, the combination of all mRNAs (mOX40L, tethered mIL-12 and cell-associated hIL-15) was the most efficacious.

TABLE 24
Efficacy of single or combination mRNA in disseminated AML

| mRNA | CR (day 80) |
|---|---|
| NST | 0/10 |
| mOX40L | 3/10 |
| hIL-15/IL-15Rα | 0/10 |
| mIL-12TM | 6/10 |
| mOX40L + hIL-15/IL-15Rα | 7/10 |
| mOX40L + mIL-12TM | 8/10 |
| mIL-12TM + hIL-15/IL-15Rα | 1/10 |
| mOX40L + hIL-15/IL-15Rα + | 8/10 |
| mIL-12TM | |

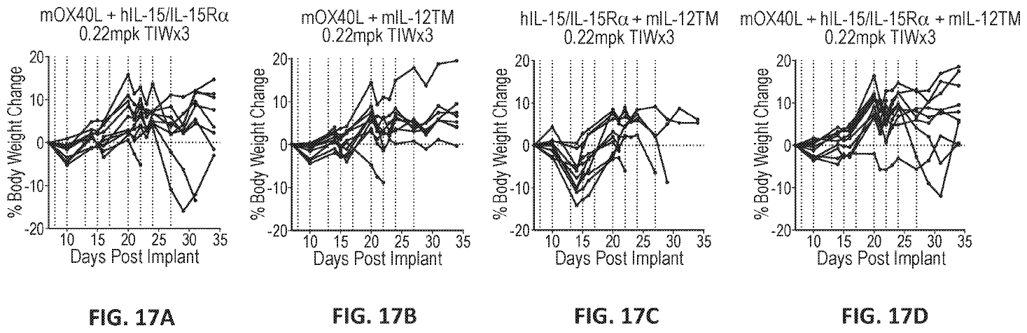

In addition, body weight in mice receiving the various combinations was evaluated. Specifically, the percentage of body weight change was determined for mice that received mOX40L+cell-associated hIL-15; mOX40L+tethered mIL-12; cell-associated hIL-15+tethered mIL-12; or mOX40L+cell-associated hIL-15+tethered mIL-12, at a dose of 0.22 mg/kg three times a week for three weeks. FIGS. 17A-17D provide the results, which shows the double combination of tethered mIL-12 and cell-associated hIL-15 causes body weight loss in the first week. Overall, the body weight loss, potent induction of cytokines, and lack of anti-leukemic response in mice that received cell-associated hIL-15 and tethered mIL-12, suggests that cytokine induction is not sufficient for efficacy, although it may be required. This example also demonstrated that OX40L is a necessary component for anti-leukemic efficacy.

### Example 9: Characterization of Efficacy of mRNA Combination Treatment in Disseminated AML Model at Varying Doses of Individual mRNAs

In the previous Examples, mRNAs encoding OX40L, tethered IL-12 and cell-associated IL-15, in combination, were administered at the same weight (mass) ratio (1:1:1). Where mRNAs encoding cell-associated IL-15 comprised an mRNA encoding hIL-15 and an mRNA encoding hIL-15Rα, the two mRNAs were formulated at a molar ratio of 1:1. To evaluate the impact of the stoichiometry of the individual components within the combination, different ratios were assessed.

Disseminated murine AML cells were implanted into mice as described in Example 6. Mice having BLI measurements of 5×10⁵ to 5×10⁶ photons per second were administered the following mRNAs at the specified ratios 9 days after implantation, at a total mRNA dose of 0.22 mg/kg three times a week for three weeks: mOX40L:cell-associated hIL-15:tethered mIL-12 weight (mass) ratio of 1:1:1; mOX40L:cell-associated hIL-15 weight (mass) ratio of 1:1; mOX40L:tethered mIL-12 weight (mass) ratio of 1:1; tethered mIL-12:cell-associated hIL-15 weight (mass) ratio of 1:1; mOX40L:cell-associated hIL-15:tethered mIL-12 weight (mass) ratio of 1:1:0.1; mOX40L:cell-associated hIL-15:tethered mIL-12 weight (mass) ratio of 1:0.1:1; or mOX40L:cell-associated hIL-15:tethered mIL-12 weight (mass) ratio of 0.1:1:1. Table 25 provides the results, which indicate that while the combination of tethered mIL-12 and cell-associated hIL-15 is not efficacious and potentially toxic, including mOX40L even at one-tenth the amount of tethered mIL-12 and cell-associated hIL-15 rescues efficacy, and also alleviates body weight loss (data not shown).

TABLE 25
Efficacy of combination mRNA ratios

| mRNA | mRNA ratios | CR at day 80 (% CR) | |
|---|---|---|---|
| mOX40L + hIL-15/IL- 15Rα | 1:1 | 9/15 | (60) |
| mOX40L + mIL-12TM | | 10/15 | (67) |
| mIL-12TM + hIL- 15/IL-15Rα | | 1/15 | (7) |
| mOX40L + hIL-15/IL- 15Rα + | 1:1:1 | 13/15 | (87) |
| mIL-12TM | 1:1:0.1 | 8/10 | (80) |
| | 1:0.1:1 | 8/10 | (80) |
| | 0.1:1:1 | 7/10 | (70) |

To further assess the dosing of the combinations of mRNAs encoding mOX40L, tethered mIL-12 and cell-associated hIL-15, total mRNA was administered at either 0.22 mg/kg or 0.11 mg/kg three times a week for two weeks. Table 26 show similar anti-tumor efficacy of the triple combination when the dose is halved. However, when the double combinations of mOX40L with tethered mIL-12 or cell-associated hIL-15 were administered at the lower dose (i.e., 0.11 mg/kg), anti-tumor efficacy is almost lost Specifically, the complete response rate is reduced from 60-80% to 20%. Thus, the triplet combination shows superior efficacy over any corresponding doublet combination in this model.

TABLE 26
Efficacy of combination mRNA dosage

| mRNA | Dose (mpk) | CR at day 52 |
|---|---|---|
| mOX40L + hIL-15/IL- 15Rα | 0.22 | 6/10 |
| | 0.11 | 2/10 |
| mOX40L + mIL-12TM | 0.22 | 8/10 |
| | 0.11 | 2/10 |
| mOX40L + hIL-15/IL-15Rα + | 0.22 | 9/10 |
| mIL-12TM | 0.11 | 7/10 |

### Example 10: Combined Treatment of Disseminated AML with mRNAs and Immune Checkpoint Inhibitors

In this example, the disseminated murine AML model described in Example 6 was used to examine the efficacy of treatment with tethered and cell-associated cytokine mRNA constructs in combination with immune checkpoint inhibitor agents.

Specifically, an anti-mPD-L¹ or anti-mCTLA-4 antibody (BioXCell) was administered to mice twice a week for two weeks (BIW×2) at a dose of 10 mg/kg, to mice receiving mRNAs encoding mOX40L, tethered mIL-12, and cell-associated hIL-15 at 0.11 mg/kg three times a week for two weeks (TIW×2). mRNAs were prepared and formulated as described in Example 6. Table 27 provides the results, which show a slight increase in anti-tumor efficacy with an anti-mCTLA-4 antibody.

TABLE 27
Efficacy combination mRNA with immune checkpoint inhibitors

| | Immune | |
|---|---|---|
| | checkpoint | CR at |
| mRNA | inhibitors | day 52 |
| mOX40L + hIL-15/IL-15Rα | — | 2/10 |
| | αmPD-L1 | 7/10 |
| | αmCTLA-4 | 9/10 |
| mOX40L + mIL-12TM | — | 2/10 |
| | αmPD-L1 | 8/10 |
| | αmCTLA-4 | 10/10 |
| mOX40L + hIL-15/IL-15Rα + | — | 7/10 |
| mIL-12TM | αmPD-L1 | 7/10 |
| | αmCTLA-4 | 10/10 |

In a separate study, the effect of adding immune checkpoint inhibitor agents to combinations of mOX40L with tethered mIL-12 or cell-associated hIL-15 was evaluated. Specifically, mice receiving 0.11 mg/kg three times a week for 2 weeks of total mRNA (mRNA encoding mOX40L and tethered mIL-12 or cell-associated hIL-15), received an anti-mPD-L¹ or anti-mCTLA-4 antibody twice a week for two weeks (BIW×2). Table 27 provides the results, which show a significant increase in efficacy when combining doublet combinations with an immune checkpoint inhibitor.

Overall, these results indicate the addition of an immune checkpoint inhibitor to the combination of mRNAs described herein enhances anti-tumor efficacy against AML.

### Example 11: Efficacy of Tethered and Cell-Associated Cytokine mRNAs in Checkpoint Therapy Resistant Solid Tumors

To determine whether the combination of tethered and cell-associated cytokine mRNAs with costimulatory mRNAs induced anti-tumor efficacy in solid tumors, an MC38-R syngeneic colon adenocarcinoma model was utilized. The MC38-R model is immunosuppressive and resistant to immune checkpoint inhibitor treatment, in contrast to the MC38-S model which is less immunosuppressive and partially sensitive to immune checkpoint inhibitor treatment. MC38-R tumors were established subcutaneously in C₅₇BL/6 mice. mRNAs encoding mOX40L, tethered mIL-12 and/or cell-associated hIL-15, as described above, were formulated in separate LNPs comprising Compound X, and administered intratumorally to established tumors at a single dose of 10 μg total mRNA. Table 28 provides the results, indicating the triplet combination of mRNAs encoding mOX40L, tethered mIL-12 and cell-associated hIL-15 were more effective at reducing tumor volume compared to the doublet combinations of mOX40L+tethered mIL-12, mOX40L+cell-associated hIL-15, and tethered mIL-12+cell-associated hIL-15.

TABLE 28
Efficacy of combination mRNA in checkpoint
therapy resistant model

| | Dosing | |
|---|---|---|
| mRNA | schedule | CR |
| NST | QDx1 | 0 |
| mOX40L + mIL-12TM | | 1/14 |
| mOX40L + hIL-15/IL-15Rα | | 0 |
| hIL-15/IL-15Rα + mIL-12TM | | 2/14 |
| mOX40L + hIL-15/IL-15Rα + | | 6/14 |
| mIL-12TM | | |
| NST | Q7Dx3 | 0 |
| mOX40L + mIL-12TM | | 2/14 |
| mOX40L + hIL-15/IL-15Rα | | 0 |
| hIL-15/IL-15Rα + mIL-12TM | | 5/14 |
| mOX40L + hIL-15/IL-15Rα + | | 9/14 |
| mIL-12TM | | |

Notably, the enhanced efficacy of the triplet combination compared to the doublet combination is in contrast to the efficacy observed in the C1498 model (see Table 19). While effective in both models, in this experiment the triplet was significantly more effective than the doublet combination of mOX40L and tethered mIL-12.

Next, the efficacy of multiple dosing of the combination of mRNAs in the MC38-R model was assessed. Specifically, LNPs comprising Compound X and encapsulating mRNAs encoding mOX40L, tethered mIL-12, and/or cell-associated hIL-15 were administered intratumorally at a dose of 10 μg total mRNA once a week for 3 weeks (Q7Dx3). Table 28 provides the results, which indicate multiple doses of the triplet combination of mOX40L, tethered mIL-12, and cell-associated hIL-15 was more effective compared to a single dose. In addition, the efficacy of the doublet combination of tethered mIL-12 and cell-associated hIL-15 was enhanced with multiple dosing. Notably, the anti-tumor efficacy of the triplet combination remained more effective than the doublet combinations.

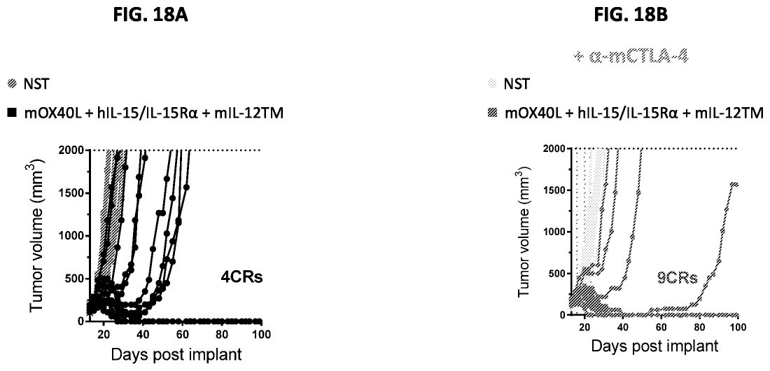

To further asses the efficacy of the triplet combination in a solid tumor, combinations with a checkpoint inhibitor were evaluated. Specifically, a single dose (i.e., 10 μg total mRNA) of LNPs comprising Compound X and encapsulating mRNAs encoding mOX40L, tethered mIL-12, and cell-associated hIL-15 was administered to mice having MC38-R tumors, in combination with an anti-mCTLA-4 antibody (BioXcell). FIGS. 18A-18B show enhanced efficacy of the triplet combination when administered with an anti-mCLTA-4 antibody.

To determine whether the LNP formulation confers efficacy of the triplet combination, LNPs comprising Compound X were compared to LNPs comprising Compound 428 and Compound X. mRNAs encoding mOX40L, tethered mIL-12, and cell-associated hIL-15 were formulated in separate LNPs and administered to mice having MC38-R tumors at a single dose or multiple doses (i.e., once a week for three weeks) of 10 μg total mRNA. Table 29 shows comparable anti-tumor efficacy between the two LNP formulations for the single dose, and an improved efficacy with both LNPs comprising Compound X with or without Compound 428 for the multiple dosing.

TABLE 29
Efficacy of mRNA combination in different LNP formulations

| | | Dosing | |
|---|---|---|---|
| LNP formulation | mRNA | schedule | CR |
| Compound X/DMG | NST | Q7Dx3 | 0 |
| | | QDx1 | 4/13 |
| | mOX40L + hIL-15/ | Q7Dx3 | 10/13 |
| | IL-15Rα + | | |
| | mIL-12TM | | |
| Compound X/ | NST | Q7Dx3 | 0 |
| Compound 428 | mOX40L + hIL-15/ | QDx1 | 5/13 |
| | IL-15Rα + | | |
| | mIL-12TM | Q7Dx3 | 9/13 |

Overall, these results indicated the combination of mRNAs encoding mOX40L, tethered mIL-12, and cell-associated hIL-15 were effective in solid tumors at single and multiple dosing regimens.

### Example 12: Abscopal Effect of Tethered and Cell-Associated Cytokine mRNAs in Solid Tumors

To further assess the anti-tumor efficacy of the combination of mRNAs encoding mOX40L, tethered mIL-12, and cell-associated hIL-15 in solid tumors, the abscopal effect of the combination was evaluated. Specifically, it was tested whether untreated tumors would benefit from the anti-tumor effects seen in treated tumors.

In one study, the CT26 syngeneic colon cancer model was utilized. CT26 is one of the most extensively studied syngeneic mouse tumor models, used in the screening for and evaluation of small molecule cytotoxic agents and biological response modifiers for use in cancer immunotherapy. CT26 tumors cells were injected into each flank of BALB/c mice to establish subcutaneous tumors. To analyze the abscopal effect of the triplet combination of mRNAs, only one tumor was administered mRNAs encoding mOX40L, tethered mIL-12, and cell-associated hIL-15 formulated in separate LNPs comprising Compound X (“treated”). The mRNAs were administered either at a single dose or at multiple doses (one a week for three weeks, “Q7Dx3”) of 5 μg total mRNA, at a 1:1:1 weight (mass) ratio of mOX40L:tethered mIL-12:cell-associated hIL-15. Table 30 shows both treated and untreated tumors were responsive to the triplet combination of mRNAs, regardless of the dosing schedule.

TABLE 30
Abscopal effect of mRNA combination

| | | Dosing | CR | |
|---|---|---|---|---|
| Tumor type | mRNA | schedule | treated | distal |
| CT26 | NST | Q7Dx3 | 0 | 0 |
| | mOX40L + hIL-15/ | QDx1 | 11/20 | 11/20 |
| | IL-15Rα + mIL-12TM | Q7Dx3 | 11/20 | 11/20 |
| A20 | NST | Q7Dx3 | 0 | 0 |
| | mOX40L + hIL-15/ | QDx1 | 8/20 | 8/20 |
| | IL-15Rα + mIL-12TM | Q7Dx3 | 12/20 | 12/20 |

In a second study, A20 mouse B-cell lymphoma cells (A20, ATCC No. TIB-208; ATCC, Manassas, Va.) were injected in BALB/c mice to generate subcutaneous tumors. Tumors were established in both flanks of the mice to evaluate abscopal effects. mRNAs encoding mOX40L, tethered mIL-12, and cell-associated hIL-15 were formulated in separate LNPs comprising Compound X and administered to one of the tumors (“treated”). mRNAs were administered intratumorally at either a single dose or multiple doses (i.e., once a week for two weeks) of 5 μg total mRNA, at a 1:1:1 weight (mass) ratio of mOX40L:tethered mIL-12:cell-associated hIL-15. Table 30 shows both treated and untreated tumors were responsive to the triplet combination of mRNAs, regardless of dosing schedule.

### Example 13: Immune Activation by Administration of Tethered and Cell-Associated Cytokine mRNAs in Mice Inoculated with AML Cells

In this example, immune activation after treatment with the combination of mRNAs encoding mOX40L, tethered mIL-12, and cell-associated hIL-15 were studied in mice inoculated with GFP+AML cells, as described in Example 6 above. Untreated, the AML cells proliferate in various hematopoietic tissues, as measured by increased BLI signal. On day 12 post-AML implant, the tumor-bearing mice were treated with the combination of mOX40L, tethered mIL-12, and cell-associated hIL-15 mRNAs encapsulated in a lipid nanoparticle at a weight (mass) ratio of 1:1:1, administered intravenously at a dose of 0.22 mg/kg/dose three times a week (with at least 48 hours in between doses) for two weeks (TIW×2). Lipid nanoparticles were formulated comprising: 50% ionizable amino lipid (Compound X), 10% phospholipid; 38.5% cholesterol and 1.5% PEG-modified lipid, wherein the PEG-modified lipid is Compound 428. As a control, similarly inoculated mice were administered control mRNA (NST) encapsulated in the same lipid nanoparticle formulation at the same dose level and schedule.

To assess how this treatment combination affects immune cell populations, tissues (spleen, bone marrow, and lymph node) and peripheral blood were collected at 24 hours (24 hours post-1^{st} dose), 6 days (24 hours post-3^{rd} dose) and 13 days (24 hours post-6^{th} dose) and analyzed by flow cytometry Immune cell populations were identified out of live CD45+ cells using the following gating strategies:

NK cells: CD3− CD49b+;

NKT cells: CD3+CD49b+;

CD4+ T cell: CD3+CD49b− CD8− CD4+Foxp3−;

CD8+ T cell: CD3+CD49b− CD8+CD4−;

CD8+ cDC1: CD11c+MHC II+B220− CD8+;

CD103+ cDC1: CD11c+MHC II+B220− CD103+;

cDC2: CD11c+, MHC II+B220− Ly6C− CD24+;

iDC: CD11c+, MHC II+B220− CD11b+Ly6C+.

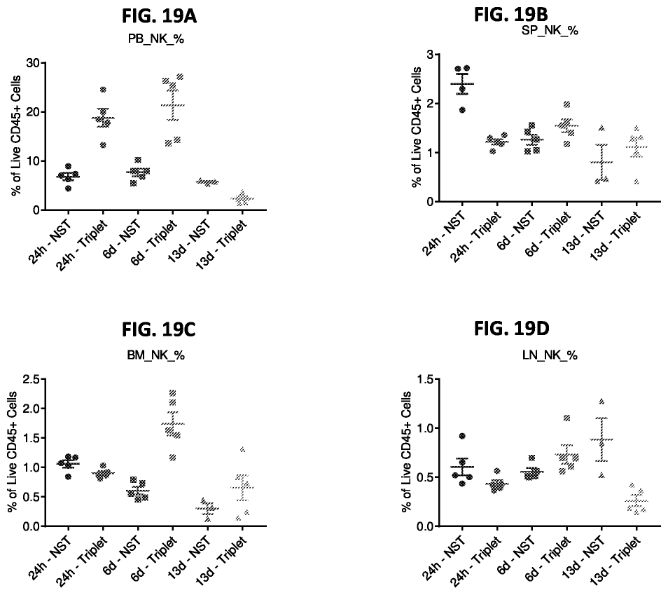

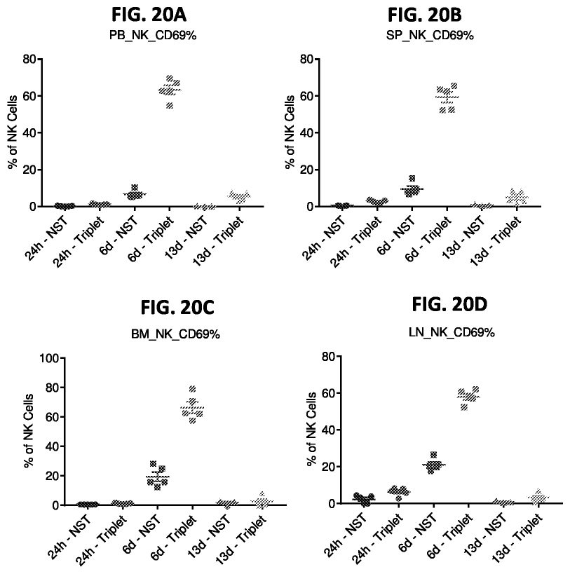

As early as 24 hours post-1st dose, the total number of NK cells increased in peripheral blood of mice administered the combination of mRNAs encoding mOX40L, tethered mIL-12, and cell-associated hIL-15 relative to blood from mice given control mRNA. However, by 24 hours post-6th dose (i.e., 13d), NK cell numbers declined in both peripheral blood and immune tissues (spleen, bone marrow, and lymph nodes), indicating that the NK cell levels had returned to baseline (data not shown). The same results were observed when the NK cells were analyzed as a percentage of live CD45+ cells. (FIGS. 19A-19D). Further study of NK cell proliferation using the marker Ki-67, showed that NK cells exhibited increased proliferation as early as 24 hours post-first dose in peripheral blood, spleen, bone marrow, and to a lesser extent, in inguinal lymph nodes (data not shown). NK cell activation was also evaluated using the CD25 (IL-2Rα) (data not shown) and CD69 (FIGS. 20A-20D) markers. The results show that the combination of mRNAs encoding mOX40L, tethered mIL-12 and cell-associated hIL-15 induced activation, proliferation and expansion of NK cells in multiple hematopoietic tissues in treated mice.

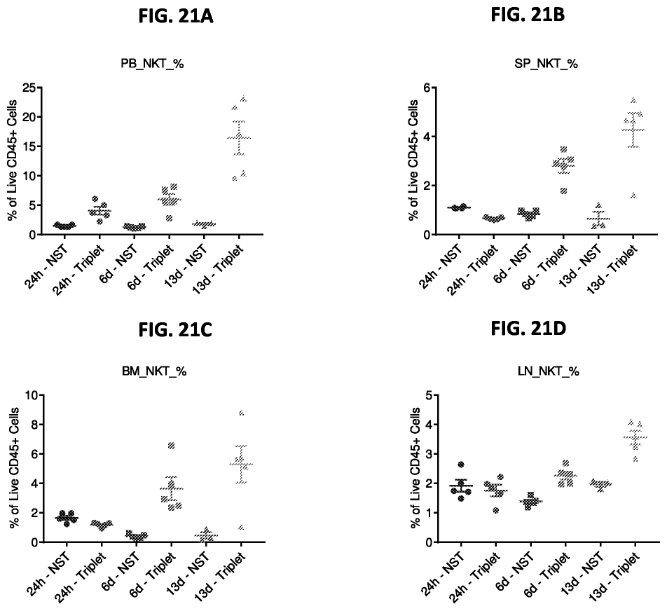

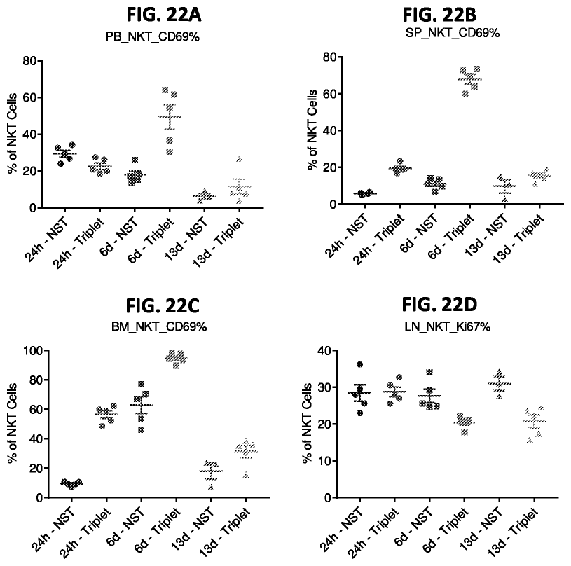

The dynamics of NKT cell response were also studied. Specifically, total NKT cell numbers (data not shown) and NKT cells as a percentage of CD45+ cells (FIGS. 21A-21D) were analyzed and NKT cell expansion was observed. An assessment of Ki-67 expression showed that NKT cell proliferation was also increased (data not shown). Expression of activation markers CD25 (data not shown) and CD69 (FIGS. 22A-22D) was also increased in triplet-treated mice relative to control mice (data not shown).

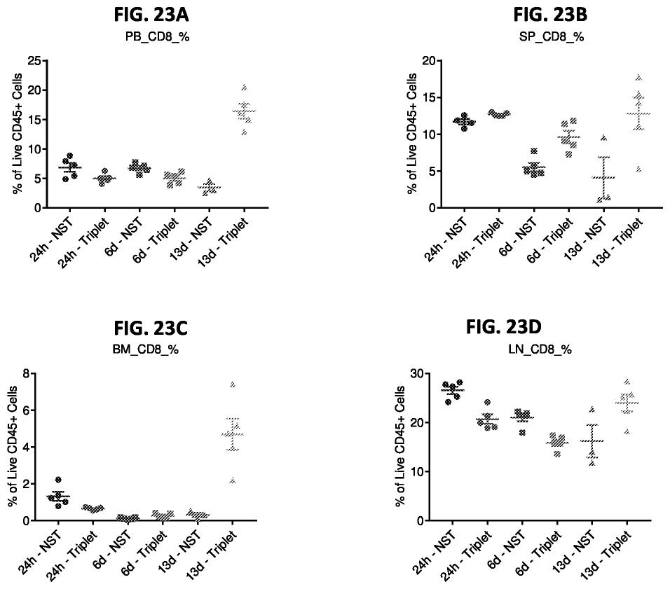

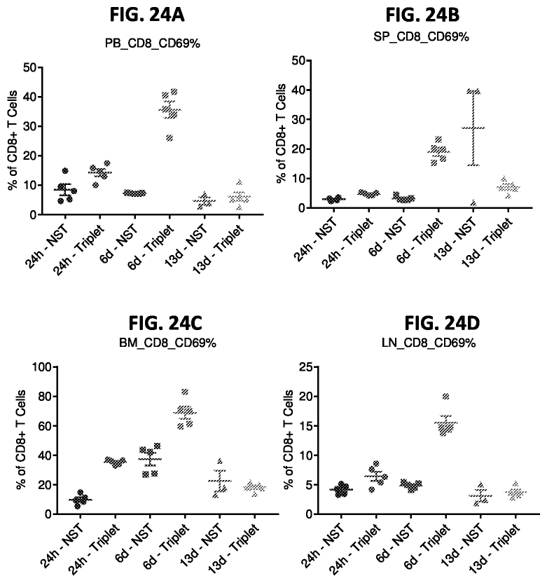

Treatment with the combination of mRNAs encoding mOX40L, tethered mIL-12 and cell-associated hIL-15 also induced expansion of CD8+ T cells as measured by absolute numbers or as a percentage of live CD45+ cells (FIGS. 23A-23D) by 6 days (24 hours post-3^{rd} dose) and continued up to 13 days (24 hours post-6^{th} dose). CD8+ T cell proliferation (Ki-67 expression) was also increased by 6 days and remained elevated through 13 days of the study (data not shown). Similarly, activation markers CD25 (data now shown) and CD69 (FIGS. 24A-24D) were also upregulated by 6 days and continued to increase up to 13 days.

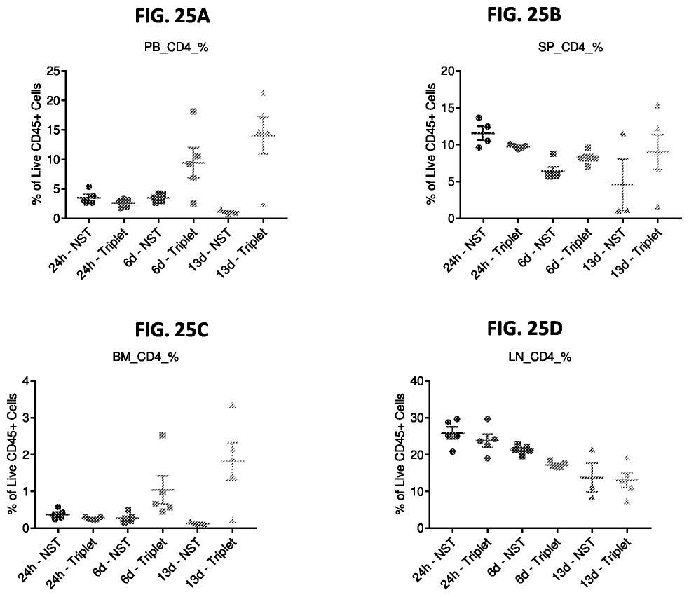

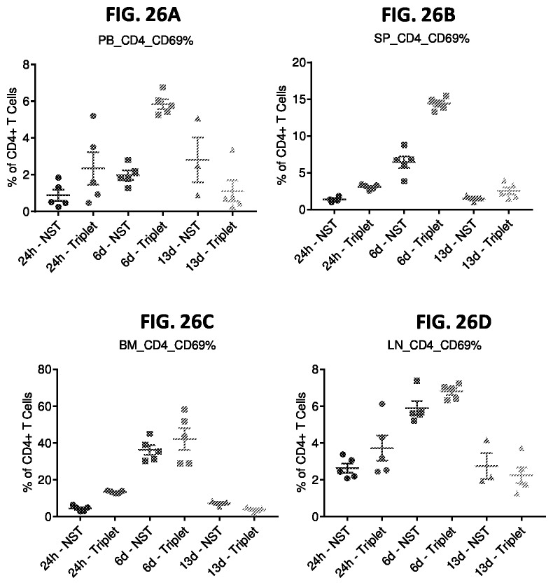

Treatment with the combination of mRNAs encoding mOX40L, tethered mIL-12 and cell-associated hIL-15 also resulted in an expansion of CD4+ T cells as measured by absolute numbers or as a percentage of live CD45+ cells (FIGS. 25A-25D), increased CD4+ T cells proliferation (data not shown), and upregulation of CD4+ T cell activation markers (FIGS. 26A-26D).

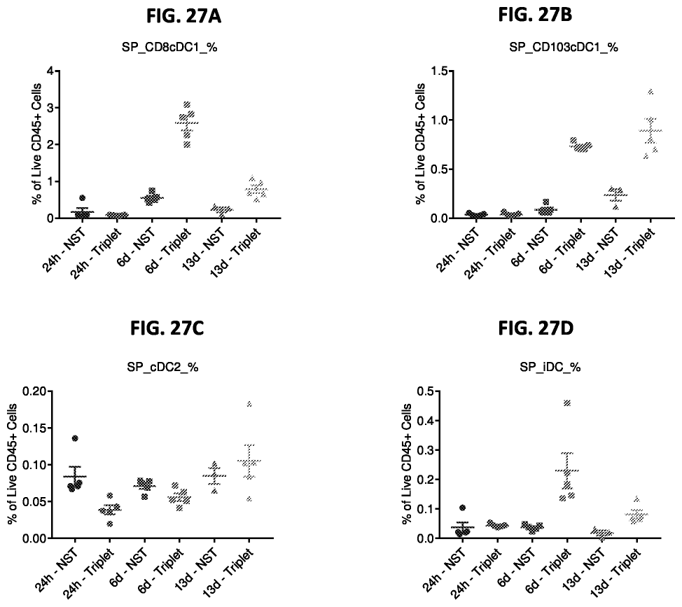

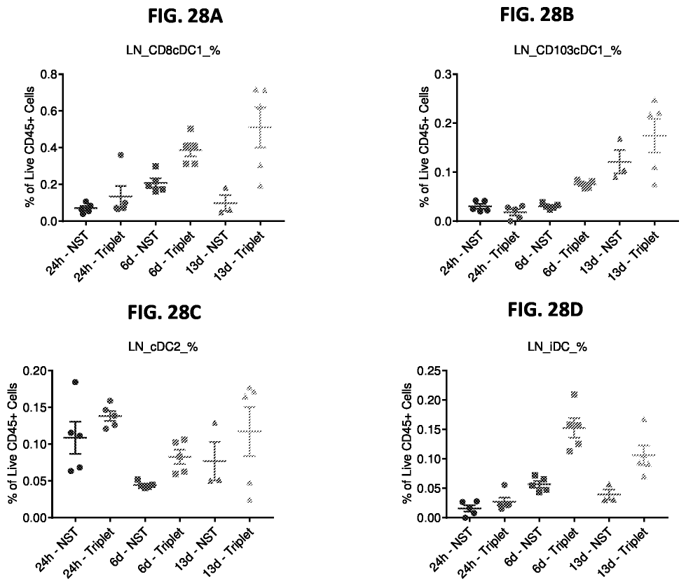

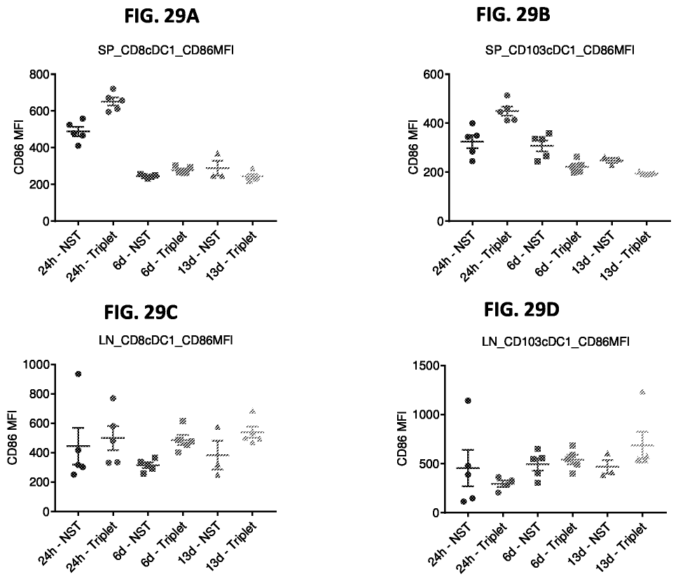

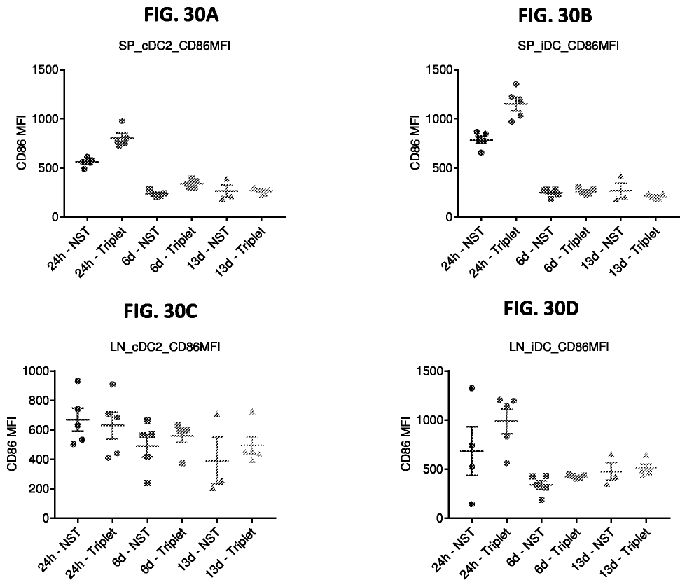

Dendritic cell populations (CD8+ cDC1, CD103+ cDC1, cDC2, and iDC) also increased in cell numbers and as a percentage of live CD45+ cells, as measured in spleen (FIGS. 27A-27D,) and lymph nodes (FIGS. 28A-28D, respectively). The DC populations also showed an early increase in expression of the CD86 maturation marker that slowly tapered off back to baseline levels by 13 days (FIGS. 29A-29D, 30A-30D). This study shows that the combination of mRNAs encoding mOX40L, tethered mIL-12 and cell-associated hIL-15 are immunostimulatory, leading to an increase in innate and adaptive immune cells (CD4+ T cells, CD8+ T cells, NK cells, NKT cells, CD8+ cDC1s, CD103+cDC1s, cDC2s, and iDCs) numbers and expression of proliferation, activation, and maturation markers.

### Example 14: Efficacy of Tethered and Cell-Associated Cytokine mRNAs in the Treatment of Disseminated AML in Batf3 KO mice

The efficacy of mRNAs encoding OX40L, tethered IL-12 and cell-associated IL-15 was assessed Batf3knockout (KO) mice to evaluate the role of dendritic cells in mediating the anti-leukemic response. The Batf3 KO mice lack exons 1-2 of the Batf3 (basic leucine zipper transcription factor, ATF-like) gene. Batf3 is highly expressed in CD11c+ CD8α+ conventional dendritic cells (cDCs), which play a role in antigen cross-presentation. Batf3 KO mice lack CD8α+ dendritic cells, are deficient in TLR-3 induced IL-12 production, and also lack CD103+CD11b− DCs in the lung, intestines, mesenteric lymph nodes (MLNs), dermis, and skin-draining lymph nodes. Batf3 KO mice are defective in CD8+ T cell priming, and are not able to reject highly immunogenic syngeneic tumors. (Hildner K et al., Science 322(5904):1097-1100 (2008)).

In this study, mRNAs encoding mOX40L, tethered mIL-12, and cell-associated hIL-15 were intravenously administered to either C57BL/6 or Batf3 KO mice according to the study design as summarized in Table 31:

TABLE 31
Efficacy of mRNAs encoding mOX40L, tethered
mIL-12, and cell-associated hIL-15

| | | mRNA dose | |
|---|---|---|---|
| Group | mRNA | (mg/kg) | CR |
| 1 (C57BL/6) | Untreated | 0.22 | 0/10 |
| 2 (C57BL/6) | NST (1:1:1) | 0.22 | 0/10 |
| 3 (C57BL/6) | mOX40L + mIL-12TM + hIL-15/ | 0.22 | 7/10 |
| | IL-15Rα (1:1:1) | | |
| 4 (Batf3 KO) | NST | 0.22 | 0/10 |
| 5 (Batf3 KO) | mOX40L + mIL-12TM + hIL-15/ | 0.22 | 6/10 |
| | IL-15Rα (1:1:1) | | |

The mice were inoculated with AML cells expressing GFP at day 0, as described in Example 6 above. Once the AML cells were established, mRNAs encapsulated in LNP as described in Example 13, were administered intravenously three times a week for two weeks (TIW×2). Mice were imaged twice weekly to monitor disease burden. AML cells continued to expand in untreated and control mRNA (NST) treated mice. In both C57BL/6 and Batf3 KO mice, administration of LNP encapsulating the mRNAs encoding mOX40L, tethered mIL-12 and cell-associated hIL-15 reduced AML disease burden (e.g., decreased BLI signal). Table 31 indicates the complete responders in both C57BL/6 (CR=7) and Batf3 KO (CR=6) groups.

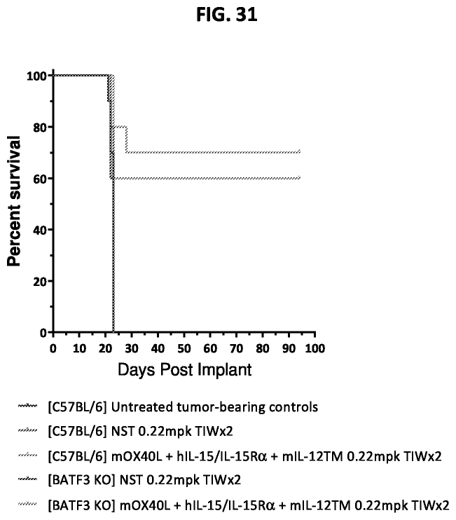

These results show that although administration of mRNAs encoding OX40L, tethered IL-12 and cell-associated IL-15 resulted in the expansion and activation of various cross-presenting DCs (see e.g., Example 13), the ability of the mRNA combination to affect anti-tumor response is not dependent on having a functional cross-presenting DC population, as shown by the suppression of AML disease burden and the increased survival through 100 days post-treatment (FIG. 31) of both C57BL/6 and Batf3 KO mice.

### Example 15: Anti-Tumor Efficacy of Tethered and Cell-Associated Cytokine mRNAs is Abrogated by IFNγ Neutralization or T Cell Depletion in the Treatment of Disseminated AML

The role of T cells in mediating the efficacy of the combination of mRNAs encoding mOX40L, tethered mIL-12, and cell-associated hIL-15 was further evaluated in a murine AMLstudy as described in the Examples above. Briefly, C57BL/6 mice were given intravenous tail-vein injection of 1×10⁶ MLL-AF9 cells (Day 0). At day 9 post-AML implant, tumor-bearing mice (n=15) were treated with combination of mOX40L, tethered mIL-12, and cell-associated hIL-15 mRNAs, encapsulated in a lipid nanoparticle at a weight (mass) ratio of 1:1:1, and administered intravenously at a dose of 0.11 mg/kg/dose three times a week for two weeks (TIW×2). As a control, similarly inoculated mice were administered control mRNA (NST) encapsulated in a lipid nanoparticle at the same dose and on the same schedule. Beginning one day prior to initiation of mRNA administration, mice were given four intraperitoneral injections of a CD8+-depleting antibody (2.43, BP0061, BioXCell) or a Rat IgG2b isotype control (LTF-2, BP0090, BioXCell) as summarized in Table 32:

TABLE 32
mOX40L + mIL-12TM + hIL-15/IL-15Rα
mRNA in CD8+ depleted mice

| | | mRNA | Antibody | |
|---|---|---|---|---|
| | | dose | dose | |
| Group | mRNA | (mg/kg)^{a} | (mg/mL)^{b} | CR |
| 1 | NST | 0.11 | Rat IgG2b 0.5 mg | 0/15 |
| 2 | mOX40L + | TIWx2 | QDx3, 3 days off, | 10/15 |
| | mIL-12TM + | | 0.2 mg QDx1 | |
| | hIL-15/IL-15Rα | | | |
| 3 | NST | | Anti-CD8 0.5 mg | 0/15 |
| 4 | mOX40L + | | QDx3, 3 days off, | 1/15 |
| | mIL-12TM + | | 0.2 mg QDx1 | |
| | hIL-15/IL-15Rα | | | |

^{a}mRNA administered iv on days 9, 11, 14, 16, 18, and 21
^{b}antibodies administered ip on days 8, 9, 10, and 14

Administration of the anti-CD8 antibody resulted in depletion of CD8+ T cells as early as 11 days post implant (2 days post-Pt mRNA dose) and was sustained through Day 22 (data not shown). The depletion of CD8+ T cells resulted in the abrogation of the therapeutic efficacy of the combination of mRNAs encoding OX40L, tethered IL-12 and cell-associated IL-15 (Table 32). Administration of the mRNA combination along with the isotype antibody successfully reduced AML disease burden leading to 10 complete responders up to 60 days post-implantation. In contrast, CD8+ T cell-depleted mice treated with the mRNA combination were not able to control AML disease burden, demonstrated by a significant reduction in the number of responders (CR=1). The inhibition of AML progression is specific to the mRNA combination, as mice given control mRNA were not able to control AML progression, regardless of whether they were given isotype control or anti-CD8 antibodies. The results demonstrate that efficacy of mOX40L+hIL-15/IL-15Rα+mIL-12TM in controlling AML is dependent on CD8+ T cell function.

In another experiment, the role of CD4+ T cells in mediating the efficacy of mOX40L+hIL-15/IL-15Rα+mIL-12TMagainst AML was studied using an anti-CD4 antibody (GK1.5, BP0003-1, BioXCell) to deplete CD4+ T cells in mice inoculated with GFP+AML cells and treated with mOX40L+hIL-15/IL-15Rα+mIL-12TM mRNAs encapsulated in LNP. The study design is summarized in Table 33:

TABLE 33
Efficacy of mOX40L + hIL-15/IL-15Rα +
mIL-12TM in CD4+ depleted mice

| | | mRNA | Antibody | |
|---|---|---|---|---|
| | | dose | dose | |
| Group | mRNA | (mg/kg)^{a} | (mg/mL)^{b} | CR |
| 1 | NST | 0.11 | — | 0/15 |
| 2 | mOX40L + hIL-15/ | TIWx2 | | 8/15 |
| | IL-15Rα + | | | |
| | mIL-12TM | | | |
| 3 | NST | | Rat IgG2b 0.5 mg | 0/15 |
| 4 | mOX40L + hIL-15/ | | QDx3, 3 days off, | 4/15 |
| | IL-15Rα + | | 0.2 mg QDx1 | |
| | mIL-12TM | | | |
| 5 | NST | | Anti-CD4 + 0.5 mg | 0/15 |
| 6 | mOX40L + hIL-15/ | | QDx3, 3 days off, | 0/15 |
| | IL-15Rα + | | 0.2 mg QDx1 | |
| | mIL-12TM | | | |

^{a}mRNA administered iv on days 9, 11, 14, 16, 18, and 21
^{b}antibodies administered ip on days 8, 9, 10, and 14

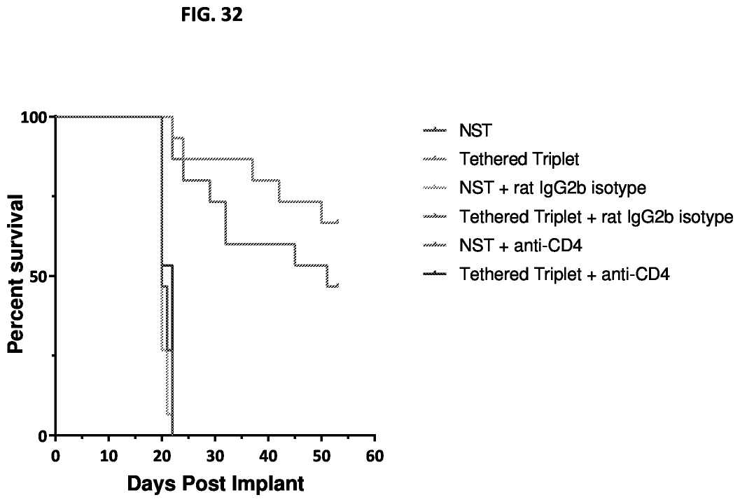

Administration of the anti-CD4 antibody resulted in depletion of CD4+ T cells as early as 11 days post implant (2 days post-1st mRNA dose) and was sustained through Day 22 (data not shown). The depletion of CD4+ T cells resulted in the abrogation of the therapeutic efficacy of the combination of mRNAs encoding mOX40L, tethered mIL-12 and cell-associated hIL-15 (Table 33). Administration of the mRNA combination successfully reduced AML disease burden in mice that were given no antibody (CR=8) or isotype antibody (CR=4). In contrast, CD4+ T cell-depleted mice treated with the mRNA combination were not able to control AML disease burden. The inhibition of AML progression is specific to treatment with the triplet, as mice given control mRNA, were not able to control AML progression, regardless of whether they were given isotype control or anti-CD4 antibodies. The survival curve of the animals up to 30 days post-implantation is shown in FIG. 32. The majority (11/15) of tumor-bearing mice that were administered mOX40L+hIL-15/IL-15Rα+mIL-12TM survived up to 30 days post implantation. In contrast, none of the CD4+ T cell-depleted mice treated with the mRNA combination survive past day 22 post-implantation. The results demonstrate that efficacy of mOX40L+hIL-15/IL-15Rα+mIL-12TM in controlling AML is also CD4+ T cell-dependent.

A further experiment was conducted to determine the role of IFNγ in mediating the anti-leukemic activity of this mRNA combination. AML-inoculated mice (n=15) were intraperitoneally administered an IFNγ neutralizing antibody (XMG1.2, BioXCell) concurrently with administration of mOX40L+hIL-15/IL-15Rα+mIL-12TM mRNAs or control mRNA (NST). The study design is summarized in Table 34:

TABLE 34
Efficacy of mOX40L + hIL-15/IL-15Rα + mIL-
12TMin anti-IFNγ treated mice

| | | mRNA | Antibody | |
|---|---|---|---|---|
| | | dose | dose | |
| Group | mRNA | (mg/kg)^{a} | (mg/mL)^{b} | CR |
| 1 | NST | 0.11 | — | 0/15 |
| | | TIWx2 | | |
| 2 | mOX40L + hIL-15/ | | | 5/15 |
| | IL-15Rα + | | | |
| | mIL-12TM | | | |
| 3 | NST | | Isotype Control | 0/15 |
| 4 | mOX40L + hIL-15/ | | Anti-HRP (HRPN) | 5/15 |
| | IL-15Rα + | | 0.5 mg TIWx2 | |
| | mIL-12TM | | | |
| 5 | NST | | Anti-IFNγ | 0/15 |
| 6 | mOX40L + hIL-15/ | | 0.5 mg TIWx2 | 0/15 |
| | IL-15Rα + | | | |
| | mIL-12TM | | | |

^{a}mRNA administered iv on days 9, 12, 14, 16, 19, and 21 after antibody administration
^{b}antibodies administered ip on days 9, 12, 14, 16, 19, and 21 before mRNA administration

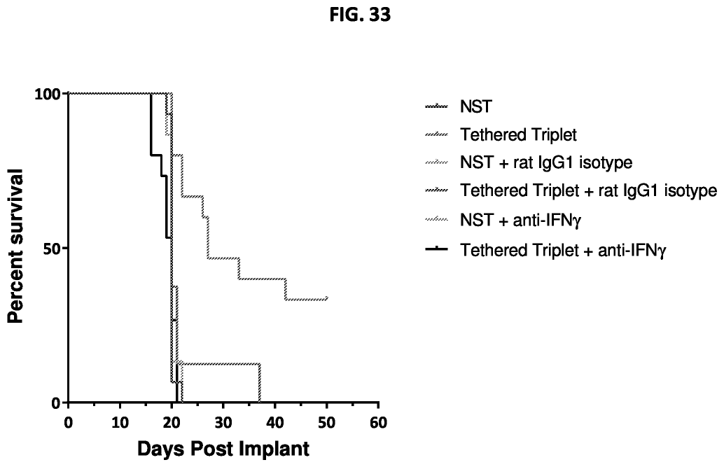

AML disease burden, as measured by BLI, showed that the anti-leukemic efficacy of mOX40L+hIL-15/IL-15Rα+mIL-12TM is severely diminished when mice were given anti-IFNγ antibodies (Table 34). Administration of hIL-15/IL-15Rα+mIL-12TM successfully reduced AML disease burden up to 60 days post-implantation in mice that were given no antibody (CR=5) or given isotype control antibody (CR=5). In contrast, anti-IFNγ antibody-treated mice were not able to control AML disease burden, even when treated with mOX40L+hIL-15/IL-15Rα+mIL-12TM. The inhibition of AML progression is mOX40L+hIL-15/IL-15Rα+mIL-12TM specific as mice given control mRNA were not able to control AML progression, regardless of whether they were given isotype control or anti-IFNγ antibodies. The survival curve of the animals up to 50 days post-implantation is shown in FIG. 33. All mice treated with mOX40L+hIL-15/IL-15Rα+mIL-12TM and with the anti-IFNγ antibody succumb to disease by day 22 post-implantation, while survival of all other mice treated with mOX40L+hIL-15/IL-15Rα+mIL-12TM was approximately 70% at the same timepoint, with about 25% survival up to 50 days post-implantation.

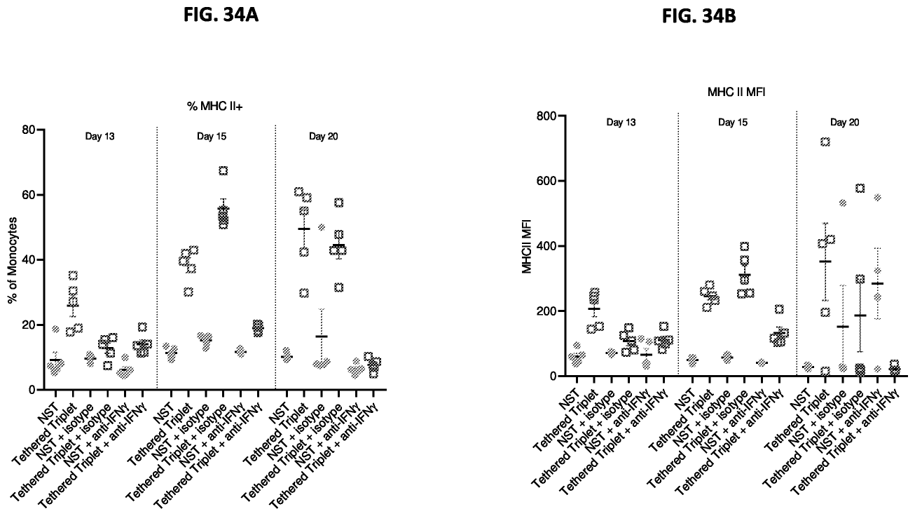

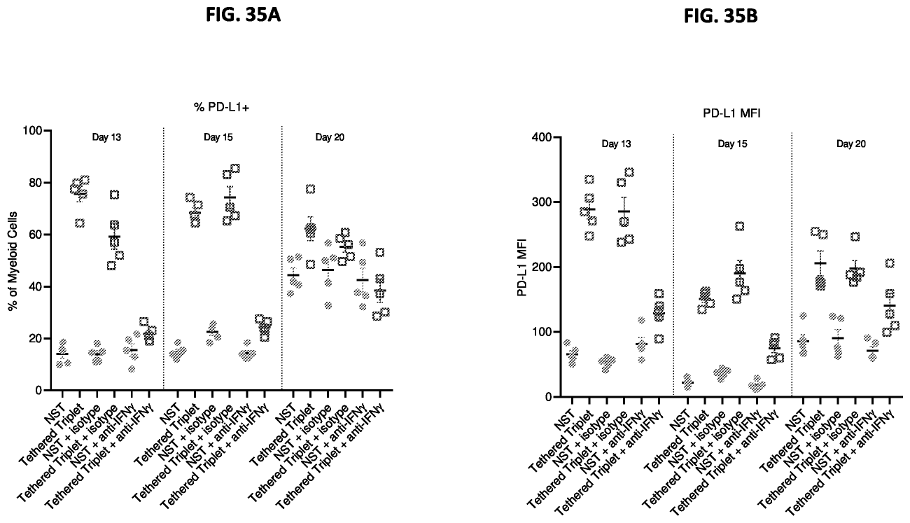

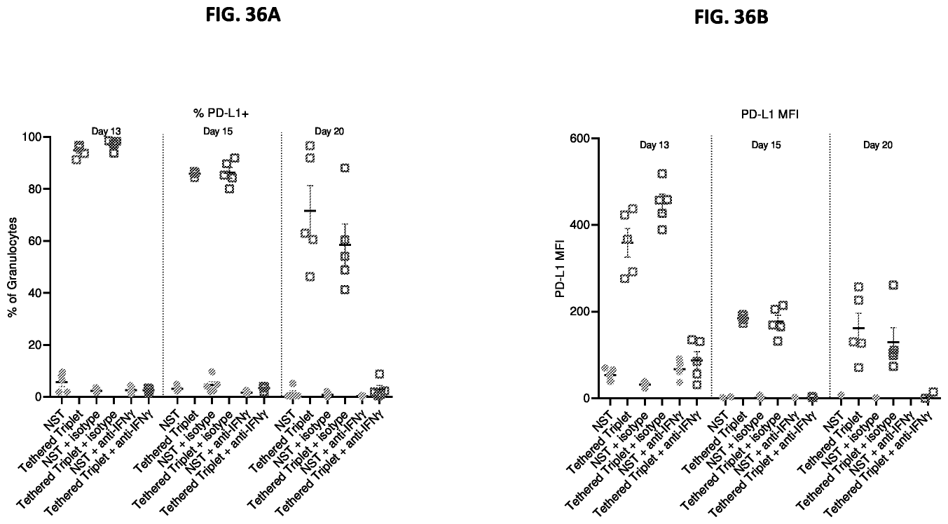

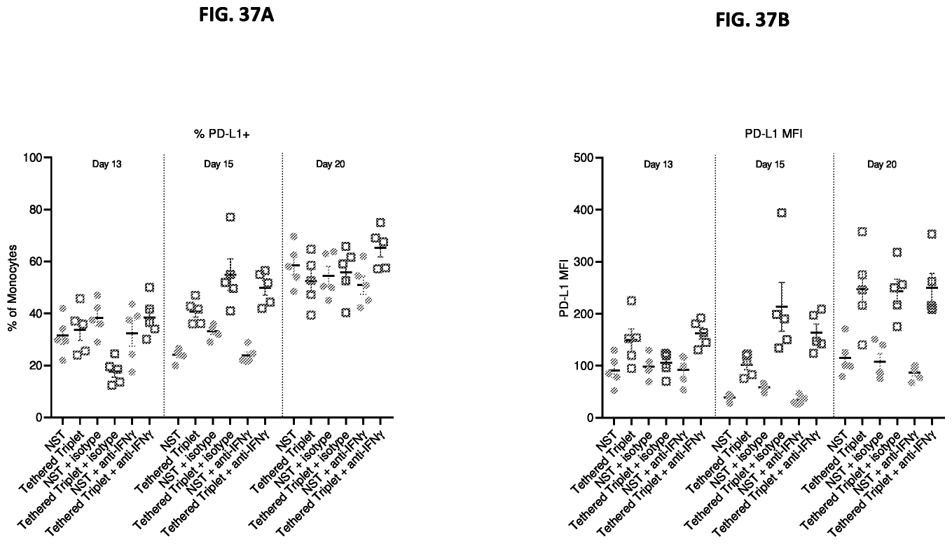

The downstream effect of IFNγ neutralization was further studied and results are shown in FIGS. 34A-34B, 35A-35B, 36A-36B, and 37A-37B. The results show that mOX40L+hIL-15/IL-15Rα+mIL-12TM treatment resulted in upregulation of markers induced by IFNγ signaling: MHC II on monocytes (FIGS. 34A-34B), and checkpoint protein PD-L¹ on myeloid cells (FIGS. 35A-35B), including both granulocytes (FIGS. 36A-36B), and monocytes (FIGS. 37A-37B). When anti-IFNγ antibody was administered, MHC II and PD-L¹ in the respective cell populations were reduced to baseline levels, demonstrating complete block of IFNγ signaling. The results demonstrate that IFNγ plays an important role in mediating the anti-leukemic efficacy of the mOX40L+hIL-15/IL-15Rα+mIL-12TM mRNA combination.

### Example 16: Administration of Tethered and Cell-Associated Cytokine mRNAs in Non-Human Primates

Non-human primates (NHPs) were administered a combination of mRNAs encoding human OX40L, tethered human IL-12, and cell-associated human IL-15 to further assess the immunostimulatory effect of the combination. Different routes of administration—subcutaneous (SC) or intravenous (IV)—and dosing schedules were evaluated in the study as summarized in Table 35:

TABLE 35
hIL-12TM + hOX40L + hIL-15/15Rα in NHP

| Group | | Dose Volume | Dose Level | Dose Conc. | | | No. of |
|---|---|---|---|---|---|---|---|
| No. | Test Material | (mL/kg) | (mg/kg/dose) | (mg/mL) | Route(s) | Dosing Days | Animals |
| 1 | Control Article | 5 (IV) | 0 | 0 | IV inf. | IV inf.: 1, 3, 5, | 3 |
| | | 1.5 (SC) | | | & SC | 8, 12, 14, and 16 | |
| | | | | | | SC: 1 and 15 | |
| 2 | hIL-12TM/ | 5 | 0.1 | 0.02 | IV inf | 1, 3, 5, 12, 14, | 3 |
| | hOX40L/hIL- | | | | | and 16 | |
| 3 | 15/IL-15Ra | 5 | 0.3 | 0.06 | | 1, 3, and 5 | 3 |
| 4 | mRNA | 5 | 1 | 0.2 | | 1 and 3 | 3 |
| 5 | | 5 | 0.3 | 0.06 | | 1, 4, and 8 | 3 |
| 6 | | 1.5 | 0.3 | 0.2 | SC | 1 and 15 | 3 |

Conc. = concentration;
IV inf. = intravenous infusion;
SC = subcutaneous injection

The dose level indicates the total amount of mRNA administered at a weight (mass) ratio of 1:1:1, e.g., 0.033, 0.1, or 0.3 mg/kg/dose each of hIL-12TM+hOX40L+hIL-15/IL-15Rα formulated in LNP as described in Example 13, for a total mRNA dose of 0.1, 0.3, or 1.0 mg/kg/dose of the combination. Serum was collected pre-dose (day 0) and at various time points post-dose to measure the induction of IFNγ and IP-10 (CXCL10) cytokine expression in each primate. Collected sera were tested at dilutions of 1:10, 1:30, 1:90 and 1:270 using commercially available Primate IFNγ ELISA kit (DY961, R&D) and Cyno CXCL10 ELISA kit (DIY1266Y-003, Kingfisher Biotech), performed according to the manufacturer's instructions.

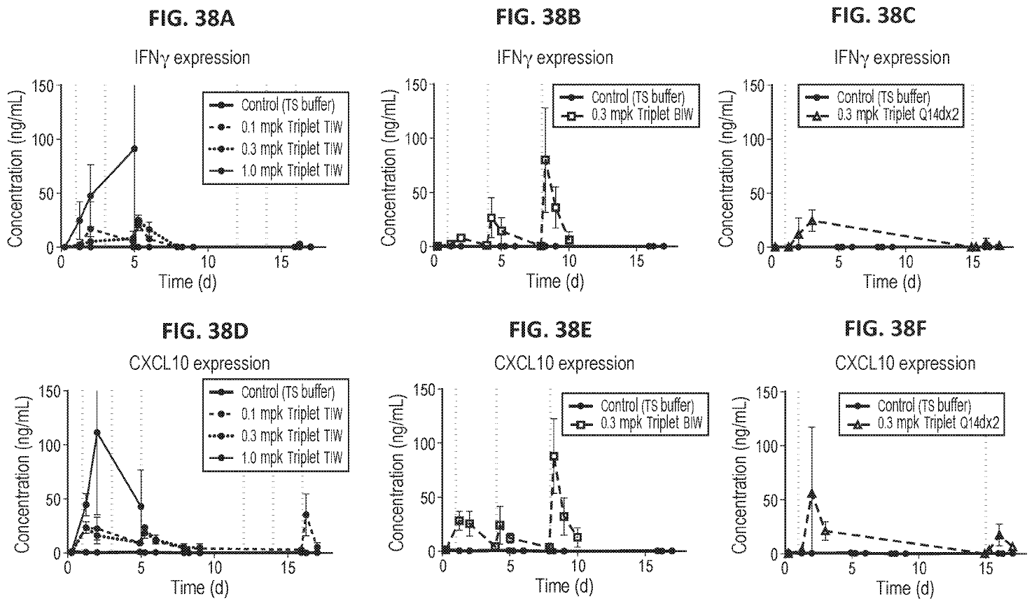

As shown in FIGS. 38A-38F, administration of the mRNA combination resulted in the induction of IFNγ (FIGS. 38A-38C) and IP-10 (CXCL¹⁰) (FIGS. 38D-38F) after each administration in a dose-dependent manner, independent of the route of administration. The study shows that the hIL-12TM+hOX40L+hIL-15/IL-15Rα mRNAs are bioactive in NHP and induce downstream cytokines associated with immune activation in a dose-dependent manner.

Tissues (spleen segment and whole femur collected 24 hours post-final dose) were also collected from the cynomolgus macaques in Group 1 (control) and Group 2 (0.1 mg/kg/dose) to further assess the local and systemic effects of hIL-12TM+hOX40L+hIL-15/IL-15Rα on the immune system Immune cell populations were identified out of live CD45+ using the following gating strategies:

NK cells: CD3− CD7+CD16+;

NKT cells: CD3+CD16+;

CD4+ T cell: CD3+CD16− CD8− CD4+;

CD8+ T cell: CD3+CD16− CD8+CD4−.

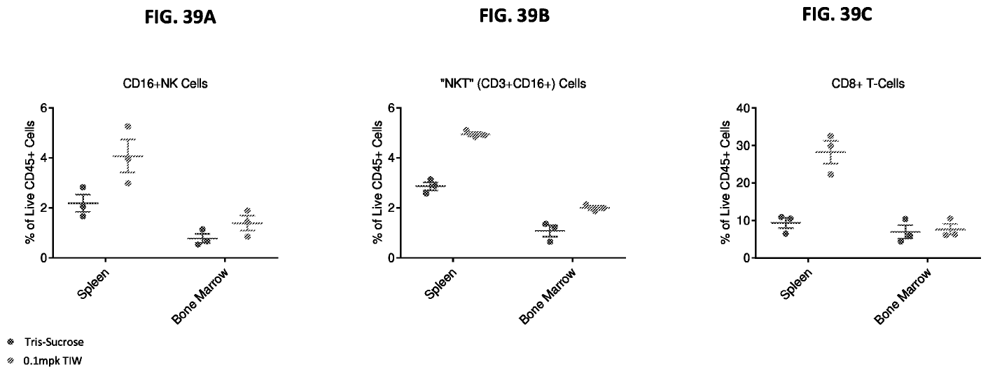

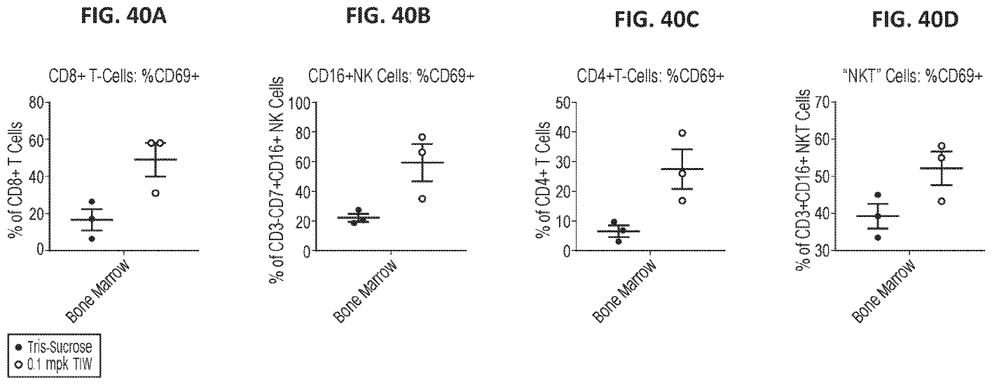

Administration of the hIL-12TM+hOX40L+hIL-15/IL-15Rα combination resulted in expansion of NK, NKT and CD8+ T cells in both the spleen and bone marrow of macaques relative to the numbers of the same cell populations in macaques treated with control article (FIGS. 39A-39C). Although the increase in the number of bone marrow immune cells (e.g., NK, CD8+ T, and NKT cells) were not as dramatic compared to the increase of the same cell populations as observed in spleen samples, further analysis of the bone marrow immune cell populations indicate that a larger population of these immune cells are activated, as measured by increased expression of CD69, compared to bone marrow cells in animals administered control article (FIGS. 40A-40D).

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

All references described herein are incorporated by reference in their entireties.

SEQUENCE LISTING SUMMARY

| SEQ | |
|---|---|
| ID | |
| NO: | DESCRIPTION |
| 1 | MERVQPLEENVGNAARPRFERNKLLLVASVIQGLGLLLCFTYICLHFSALQVSHRYPRI |
| | QSIKVQFTEYKKEKGFILTSQKEDEIMKVQNNSVIINCDGFYLISLKGYFSQEVNISLH |
| | YQKDEEPLFQLKKVRSVNSLMVASLTYKDKVYLNVTTDNTSLDDFHVNGGELILIHQNP |
| | GEFCVL |
| | OX40L (TNSFR4)-Tumor necrosis factor ligand superfamily |
| | member 4 isoform 1 [Homo sapiens] NP_003317 (bold is |
| | intracellular domain, italics is transmembrane domain, and |
| | underline is extracellular domain) |
| 2 | MVSHRYPRIQSIKVQFTEYKKEKGFILTSQKEDEIMKVQNNSVIINCDGFYLISLKGYF |
| | SQEVNISLHYQKDEEPLFQLKKVRSVNSLMVASLTYKDKVYLNVTTDNTSLDDFHVNGG |
| | ELILIHQNPGEFCVL |
| | OX40L (TNSFR4)-TNFSF4 isoform 2 [Homo sapiens]NP_001284491 |
| 3 | MEGEGVQPLDENLENGSRPRFKWKKTLRLVVSGIKGAGMLLCFIYVCLQLSSSPAKDPP |
| | IQRLRGAVTRCEDGQLFISSYKNEYQTMEVQNNSVVIKCDGLYIIYLKGSFFQEVKIDL |
| | HFREDHNPISIPMLNDGRRIVFTVVASLAFKDKVYLTVNAPDTLCEHLQINDGELIVVQ |
| | LTPGYCAPEGSYHSTVNQVPL |
| | OX40L (TNSFR4)-TNFSF4 [Mus musculus]NP_033478 |
| 4 | AUGGAAAGGGUCCAACCCCUGGAAGAGAAUGUGGGAAAUGCAGCCAGGCCAAGAUUCGA |
| | GAGGAACAAGCUAUUGCUGGUGGCCUCUGUAAUUCAGGGACUGGGGCUGCUCCUGUGCU |
| | UCACCUACAUCUGCCUGCACUUCUCUGCUCUUCAGGUAUCACAUCGGUAUCCUCGAAUU |
| | CAAAGUAUCAAAGUACAAUUUACCGAAUAUAAGAAGGAGAAAGGUUUCAUCCUCACUUC |
| | CCAAAAGGAGGAUGAAAUCAUGAAGGUGCAGAACAACUCAGUCAUCAUCAACUGUGAUG |
| | GGUUUUAUCUCAUCUCCCUGAAGGGCUACUUCUCCCAGGAAGUCAACAUUAGCCUUCAU |
| | UACCAGAAGGAUGAGGAGCCCCUCUUCCAACUGAAGAAGGUCAGGUCUGUCAACUCCUU |
| | GAUGGUGGCCUCUCUGACUUACAAAGACAAAGUCUACUUGAAUGUGACCACUGACAAUA |
| | CCUCCCUGGAUGACUUCCAUGUGAAUGGCGGAGAACUGAUUCUUAUCCAUCAAAAUCCU |
| | GGUGAAUUCUGUGUCCUU |
| | Human OX40L mRNA (ORF) |
| 5 | 5′^{7Me}GₚₚₚG2′_{OMe}GGAAAUAAGAGAGAAAAGAAGAGUAAGAAGAAAUAUAAGAGCCACCAU |
| | GGAAAGGGUCCAACCCCUGGAAGAGAAUGUGGGAAAUGCAGCCAGGCCAAGAUUCGAGA |
| | GGAACAAGCUAUUGCUGGUGGCCUCUGUAAUUCAGGGACUGGGGCUGCUCCUGUGCUUC |
| | ACCUACAUCUGCCUGCACUUCUCUGCUCUUCAGGUAUCACAUCGGUAUCCUCGAAUUCA |
| | AAGUAUCAAAGUACAAUUUACCGAAUAUAAGAAGGAGAAAGGUUUCAUCCUCACUUCCC |
| | AAAAGGAGGAUGAAAUCAUGAAGGUGCAGAACAACUCAGUCAUCAUCAACUGUGAUGGG |
| | UUUUAUCUCAUCUCCCUGAAGGGCUACUUCUCCCAGGAAGUCAACAUUAGCCUUCAUUA |
| | CCAGAAGGAUGAGGAGCCCCUCUUCCAACUGAAGAAGGUCAGGUCUGUCAACUCCUUGA |
| | UGGUGGCCUCUCUGACUUACAAAGACAAAGUCUACUUGAAUGUGACCACUGACAAUACC |
| | UCCCUGGAUGACUUCCAUGUGAAUGGCGGAGAACUGAUUCUUAUCCAUCAAAAUCCUGG |
| | UGAAUUCUGUGUCCUUUGAUAAUAGGCUGGAGCCUCGGUGGCCAUGCUUCUUGCCCCUU |
| | GGGCCUCCCCCCAGCCCCUCCUCCCCUUCCUGCACCCGUACCCCCCAAACACCAUUGUC |
| | ACACUCCAGUGGUCUUUGAAUAAAGUCUGAGUGGGCGGCAAAAAAAAAAAAAAAAAAAA |
| | AAAAAAAAAAAAAAAAAAAAAAAAAAAAAAAAAAAAAAAAAAAAAAAAAAAAAAAAAAA |
| | AAAAAAAAAAAAAAAAAAAAAUCUAG_{OH}3′ |
| | Where: A, C G & U = AMP, CMP, GMP & N1-ΨUMP, respectively; |
| | Me = methyl; p = inorganic phosphate |
| | Full-length mRNA Nucleotide sequence (5′ UTR, ORF, 3′ UTR, |
| | polyA tail) of human OX40L |
| 6 | AUGGAAAGGGUCCAACCCCUGGAAGAGAAUGUGGGAAAUGCAGCCAGGCCAAGAUUCGA |
| | GAGGAACAAGCUAUUGCUGGUGGCCUCUGUAAUUCAGGGACUGGGGCUGCUCCUGUGCU |
| | UCACCUACAUCUGCCUGCACUUCUCUGCUCUUCAGGUAUCACAUCGGUAUCCUCGAAUU |
| | CAAAGUAUCAAAGUACAAUUUACCGAAUAUAAGAAGGAGAAAGGUUUCAUCCUCACUUC |
| | CCAAAAGGAGGAUGAAAUCAUGAAGGUGCAGAACAACUCAGUCAUCAUCAACUGUGAUG |
| | GGUUUUAUCUCAUCUCCCUGAAGGGCUACUUCUCCCAGGAAGUCAACAUUAGCCUUCAU |
| | UACCAGAAGGAUGAGGAGCCCCUCUUCCAACUGAAGAAGGUCAGGUCUGUCAACUCCUU |
| | GAUGGUGGCCUCUCUGACUUACAAAGACAAAGUCUACUUGAAUGUGACCACUGACAAUA |
| | CCUCCCUGGAUGACUUCCAUGUGAAUGGCGGAGAACUGAUUCUUAUCCAUCAAAAUCCU |
| | GGUGAAUUCUGUGUCCUU |
| | TNFSF4, ORF [Homo sapiens] |
| 7 | GGCCCUGGGACCUUUGCCUAUUUUCUGAUUGAUAGGCUUUGUUUUGUCUUUACCUCCUU |
| | CUUUCUGGGGAAAACUUCAGUUUUAUCGCACGUUCCCCUUUUCCAUAUCUUCAUCUUCC |
| | CUCUACCCAGAUUGUGAAGAUGGAAAGGGUCCAACCCCUGGAAGAGAAUGUGGGAAAUG |
| | CAGCCAGGCCAAGAUUCGAGAGGAACAAGCUAUUGCUGGUGGCCUCUGUAAUUCAGGGA |
| | CUGGGGCUGCUCCUGUGCUUCACCUACAUCUGCCUGCACUUCUCUGCUCUUCAGGUAUC |
| | ACAUCGGUAUCCUCGAAUUCAAAGUAUCAAAGUACAAUUUACCGAAUAUAAGAAGGAGA |
| | AAGGUUUCAUCCUCACUUCCCAAAAGGAGGAUGAAAUCAUGAAGGUGCAGAACAACUCA |
| | GUCAUCAUCAACUGUGAUGGGUUUUAUCUCAUCUCCCUGAAGGGCUACUUCUCCCAGGA |
| | AGUCAACAUUAGCCUUCAUUACCAGAAGGAUGAGGAGCCCCUCUUCCAACUGAAGAAGG |
| | UCAGGUCUGUCAACUCCUUGAUGGUGGCCUCUCUGACUUACAAAGACAAAGUCUACUUG |
| | AAUGUGACCACUGACAAUACCUCCCUGGAUGACUUCCAUGUGAAUGGCGGAGAACUGAU |
| | UCUUAUCCAUCAAAAUCCUGGUGAAUUCUGUGUCCUUUGAGGGGCUGAUGGCAAUAUCU |
| | AAAACCAGGCACCAGCAUGAACACCAAGCUGGGGGUGGACAGGGCAUGGAUUCUUCAUU |
| | GCAAGUGAAGGAGCCUCCCAGCUCAGCCACGUGGGAUGUGACAAGAAGCAGAUCCUGGC |
| | CCUCCCGCCCCCACCCCUCAGGGAUAUUUAAAACUUAUUUUAUAUACCAGUUAAUCUUA |
| | UUUAUCCUUAUAUUUUCUAAAUUGCCUAGCCGUCACACCCCAAGAUUGCCUUGAGCCUA |
| | CUAGGCACCUUUGUGAGAAAGAAAAAAUAGAUGCCUCUUCUUCAAGAUGCAUUGUUUCU |
| | AUUGGUCAGGCAAUUGUCAUAAUAAACUUAUGUCAUUGAAAACGGUACCUGACUACCAU |
| | UUGCUGGAAAUUUGACAUGUGUGUGGCAUUAUCAAAAUGAAGAGGAGCAAGGAGUGAAG |
| | GAGUGGGGUUAUGAAUCUGCCAAAGGUGGUAUGAACCAACCCCUGGAAGCCAAAGCGGC |
| | CUCUCCAAGGUUAAAUUGAUUGCAGUUUGCAUAUUGCCUAAAUUUAAACUUUCUCAUUU |
| | GGUGGGGGUUCAAAAGAAGAAUCAGCUUGUGAAAAAUCAGGACUUGAAGAGAGCCGUCU |
| | AAGAAAUACCACGUGCUUUUUUUCUUUACCAUUUUGCUUUCCCAGCCUCCAAACAUAGU |
| | UAAUAGAAAUUUCCCUUCAAAGAACUGUCUGGGGAUGUGAUGCUUUGAAAAAUCUAAUC |
| | AGUGACUUAAGAGAGAUUUUCUUGUAUACAGGGAGAGUGAGAUAACUUAUUGUGAAGGG |
| | UUAGCUUUACUGUACAGGAUAGCAGGGAACUGGACAUCUCAGGGUAAAAGUCAGUACGG |
| | AUUUUAAUAGCCUGGGGAGGAAAACACAUUCUUUGCCACAGACAGGCAAAGCAACACAU |
| | GCUCAUCCUCCUGCCUAUGCUGAGAUACGCACUCAGCUCCAUGUCUUGUACACACAGAA |
| | ACAUUGCUGGUUUCAAGAAAUGAGGUGAUCCUAUUAUCAAAUUCAAUCUGAUGUCAAAU |
| | AGCACUAAGAAGUUAUUGUGCCUUAUGAAAAAUAAUGAUCUCUGUCUAGAAAUACCAUA |
| | GACCAUAUAUAGUCUCACAUUGAUAAUUGAAACUAGAAGGGUCUAUAAUCAGCCUAUGC |
| | CAGGGCUUCAAUGGAAUAGUAUCCCCUUAUGUUUAGUUGAAAUGUCCCCUUAACUUGAU |
| | AUAAUGUGUUAUGCUUAUGGCGCUGUGGACAAUCUGAUUUUUCAUGUCAACUUUCCAGA |
| | UGAUUUGUAACUUCUCUGUGCCAAACCUUUUAUAAACAUAAAUUUUUGAGAUAUGUAUU |
| | UUAAAAUUGUAGCACAUGUUUCCCUGACAUUUUCAAUAGAGGAUACAACAUCACAGAAU |
| | CUUUCUGGAUGAUUCUGUGUUAUCAAGGAAUUGUACUGUGCUACAAUUAUCUCUAGAAU |
| | CUCCAGAAAGGUGGAGGGCUGUUCGCCCUUACACUAAAUGGUCUCAGUUGGAUUUUUUU |
| | UUCCUGUUUUCUAUUUCCUCUUAAGUACACCUUCAACUAUAUUCCCAUCCCUCUAUUUU |
| | AAUCUGUUAUGAAGGAAGGUAAAUAAAAAUGCUAAAUAGAAGAAAUUGUAGGUAAGGUA |
| | AGAGGAAUCAAGUUCUGAGUGGCUGCCAAGGCACUCACAGAAUCAUAAUCAUGGCUAAA |
| | UAUUUAUGGAGGGCCUACUGUGGACCAGGCACUGGGCUAAAUACUUACAUUUACAAGAA |
| | UCAUUCUGAGACAGAUAUUCAAUGAUAUCUGGCUUCACUACUCAGAAGAUUGUGUGUGU |
| | GUUUGUGUGUGUGUGUGUGUGUGUAUUUCACUUUUUGUUAUUGACCAUGUUCUGCAAAA |
| | UUGCAGUUACUCAGUGAGUGAUAUCCGAAAAAGUAAACGUUUAUGACUAUAGGUAAUAU |
| | UUAAGAAAAUGCAUGGUUCAUUUUUAAGUUUGGAAUUUUUAUCUAUAUUUCUCACAGAU |
| | GUGCAGUGCACAUGCAGGCCUAAGUAUAUGUUGUGUGUGUUGUUUGUCUUUGAUGUCAU |
| | GGUCCCCUCUCUUAGGUGCUCACUCGCUUUGGGUGCACCUGGCCUGCUCUUCCCAUGUU |
| | GGCCUCUGCAACCACACAGGGAUAUUUCUGCUAUGCACCAGCCUCACUCCACCUUCCUU |
| | CCAUCAAAAAUAUGUGUGUGUGUCUCAGUCCCUGUAAGUCAUGUCCUUCACAGGGAGAA |
| | UUAACCCUUCGAUAUACAUGGCAGAGUUUUGUGGGAAAAGAAUUGAAUGAAAAGUCAGG |
| | AGAUCAGAAUUUUAAAUUUGACUUAGCCACUAACUAGCCAUGUAACCUUGGGAAAGUCA |
| | UUUCCCAUUUCUGGGUCUUGCUUUUCUUUCUGUUAAAUGAGAGGAAUGUUAAAUAUCUA |
| | ACAGUUUAGAAUCUUAUGCUUACAGUGUUAUCUGUGAAUGCACAUAUUAAAUGUCUAUG |
| | UUCUUGUUGCUAUGAGUCAAGGAGUGUAACCUUCUCCUUUACUAUGUUGAAUGUAUUUU |
| | UUUCUGGACAAGCUUACAUCUUCCUCAGCCAUCUUUGUGAGUCCUUCAAGAGCAGUUAU |
| | CAAUUGUUAGUUAGAUAUUUUCUAUUUAGAGAAUGCUUAAGGGAUUCCAAUCCCGAUCC |
| | AAAUCAUAAUUUGUUCUUAAGUAUACUGGGCAGGUCCCCUAUUUUAAGUCAUAAUUUUG |
| | UAUUUAGUGCUUUCCUGGCUCUCAGAGAGUAUUAAUAUUGAUAUUAAUAAUAUAGUUAA |
| | UAGUAAUAUUGCUAUUUACAUGGAAACAAAUAAAAGAUCUCAGAAUUCACUAAAAAAAA |
| | AAA |
| | OX40L-TNFSF4, transcript variant 1, mRNA NM_003326 |
| 8 | GGGAAAUAAGAGAGAAAAGAAGAGUAAGAAGAAAUAUAAGAGCCACCAUGGAAAGGGUC |
| | CAACCCCUGGAAGAGAAUGUGGGAAAUGCAGCCAGGCCAAGAUUCGAGAGGAACAAGCU |
| | AUUGCUGGUGGCCUCUGUAAUUCAGGGACUGGGGCUGCUCCUGUGCUUCACCUACAUCU |
| | GCCUGCACUUCUCUGCUCUUCAGGUAUCACAUCGGUAUCCUCGAAUUCAAAGUAUCAAA |
| | GUACAAUUUACCGAAUAUAAGAAGGAGAAAGGUUUCAUCCUCACUUCCCAAAAGGAGGA |
| | UGAAAUCAUGAAGGUGCAGAACAACUCAGUCAUCAUCAACUGUGAUGGGUUUUAUCUCA |
| | UCUCCCUGAAGGGCUACUUCUCCCAGGAAGUCAACAUUAGCCUUCAUUACCAGAAGGAU |
| | GAGGAGCCCCUCUUCCAACUGAAGAAGGUCAGGUCUGUCAACUCCUUGAUGGUGGCCUC |
| | UCUGACUUACAAAGACAAAGUCUACUUGAAUGUGACCACUGACAAUACCUCCCUGGAUG |
| | ACUUCCAUGUGAAUGGCGGAGAACUGAUUCUUAUCCAUCAAAAUCCUGGUGAAUUCUGU |
| | GUCCUUUGAUAAUAGGCUGGAGCCUCGGUGGCCAUGCUUCUUGCCCCUUGGGCCUCCCC |
| | CCAGCCCCUCCUCCCCUUCCUGCACCCGUACCCCCCAAACACCAUUGUCACACUCCAGU |
| | GGUCUUUGAAUAAAGUCUGAGUGGGCGGC |
| | mRNA sequence: Human OX40L with 5′-UTR, 3′-UTR, and miR-122 |
| | binding site |
| 9 | AUGGAGCGGGUGCAGCCCCUGGAGGAGAACGUGGGCAACGCCGCUCGGCCACGGUUCGA |
| | GCGGAACAAGCUGCUGCUGGUGGCUAGCGUGAUCCAGGGCCUGGGCCUGCUGCUGUGCU |
| | UCACCUACAUCUGCCUGCACUUCAGCGCCCUGCAGGUGAGCCACCGGUAUCCCCGGAUC |
| | CAGAGCAUCAAGGUGCAGUUCACCGAGUACAAGAAGGAGAAGGGCUUCAUCCUGACCAG |
| | CCAGAAGGAGGACGAGAUCAUGAAGGUGCAGAACAACAGCGUGAUCAUCAACUGCGACG |
| | GCUUCUACCUGAUCAGCCUGAAGGGCUACUUCAGCCAGGAGGUGAACAUCAGCCUGCAC |
| | UACCAGAAGGACGAGGAGCCCCUGUUCCAGCUGAAGAAGGUGCGGAGCGUGAACAGCCU |
| | GAUGGUGGCCAGCCUGACCUACAAGGACAAGGUGUACCUGAACGUGACCACCGACAACA |
| | CCAGCCUGGACGACUUCCACGUGAACGGCGGCGAGCUGAUCCUGAUCCACCAGAACCCC |
| | GGCGAGUUCUGCGUGCUG |
| | mRNA open reading frame sequence 1 for Human OX40L |
| 10 | AUGGAAAGGGUCCAACCCCUCGAAGAGAACGUGGGAAACGCAGCCAGGCCAAGAUUCGA |
| | GAGGAACAAGCUAUUGCUCGUGGCCUCAGUAAUUCAGGGACUCGGGUUACUCCUUUGCU |
| | UCACCUACAUCUGCUUGCACUUCAGUGCUCUGCAGGUAUCACAUCGGUAUCCUCGAAUU |
| | CAAAGUAUCAAAGUACAAUUUACCGAAUAUAAGAAGGAGAAAGGUUUCAUCCUCACUUC |
| | CCAGAAGGAGGAUGAAAUCAUGAAGGUGCAGAACAACUCAGUCAUCAUCAACUGUGAUG |
| | GGUUUUAUCUCAUCUCCCUGAAGGGCUACUUCUCCCAGGAAGUCAACAUUAGCCUUCAU |
| | UACCAGAAGGAUGAGGAGCCCCUCUUCCAACUGAAGAAGGUCAGGUCUGUCAACUCCUU |
| | GAUGGUAGCCUCUCUGACUUACAAAGACAAAGUCUACUUGAAUGUGACCACUGACAAUA |
| | CCUCCCUGGAUGACUUCCAUGUGAAUGGCGGAGAACUGAUUCUUAUCCAUCAGAAUCCU |
| | GGUGAAUUCUGUGUCCUU |
| | mRNA open reading frame sequence 2 for Human OX40L |
| 11 | AUGGAGCGGGUGCAGCCCCUGGAGGAGAACGUGGGCAACGCCGCUCGGCCACGGUUCGA |
| | GCGGAACAAGCUGCUGCUGGUGGCUAGCGUGAUCCAGGGCCUGGGCCUGCUGCUGUGCU |
| | UCACCUACAUCUGCCUGCACUUCAGCGCCCUGCAGGUGAGCCACCGGUAUCCCCGGAUC |
| | CAGAGCAUCAAGGUGCAGUUCACCGAGUACAAGAAGGAGAAGGGCUUCAUCCUGACCAG |
| | CCAGAAGGAGGACGAGAUCAUGAAGGUGCAGAACAACAGCGUGAUCAUCAACUGCGACG |
| | GCUUCUACCUGAUCAGCCUGAAGGGCUACUUCAGCCAGGAGGUGAACAUCAGCCUGCAC |
| | UACCAGAAGGACGAGGAGCCCCUGUUCCAGCUGAAGAAGGUGCGGAGCGUGAACAGCCU |
| | GAUGGUGGCCAGCCUGACCUACAAGGACAAGGUGUACCUGAACGUGACCACCGACAACA |
| | CCAGCCUGGACGACUUCCACGUGAACGGCGGCGAGCUGAUCCUGAUCCACCAGAACCCC |
| | GGCGAGUUCUGCGUGCUG |
| | mRNA open reading frame sequence 3 for Human OX40L |
| 12 | AGGAAAUAAGAGAGAAAAGAAGAGUAAGAAGAAAUAUAAGACCCCGGCGCCGCCACC |
| | 5′UTR |
| 13 | MAPRRARGCRTLGLPALLLLLLLRPPATRGITCPPPMSVEHADIWVKSYSLYSRERYIC |
| | NSGFKRKAGTSSLTECVLNKATNVAHWTTPSLKCIRDPALVHQRPAPPSTVTTAGVTPQ |
| | PESLSPSGKEPAASSPSSNNTAATTAAIVPGSQLMPSKSPSTGTTEISSHESSHGTPSQ |
| | TTAKNWELTASASHQPPGVYPQGHSDTTVAISTSTVLLCGLSAVSLLACYLKSRQTPPL |
| | ASVE MEAMEALPVTWGTSSRDEDLENCSHHL |
| | Full Length IL15R Amino Acid Sequence (Wild Type). Signal |
| | peptide is italicized, the sushi domain of the wild-type |
| | IL15Rα is double underlined. |
| 14 | AUGGCUCCCCGCCGCGCGCGAGGCUGUCGCACCCUCGGACUUCCUGCACUCUUGCUUUU |
| | GCUCCUCCUUAGACCCCCUGCAACCAGAGGGAUAACCUGUCCACCUCCAAUGAGCGUCG |
| | AGCACGCAGACAUUUGGGUGAAAUCAUACAGUCUGUACAGUAGAGAGCGGUACAUCUGC |
| | AACAGUGGGUUUAAAAGAAAAGCAGGCACUUCAUCUCUGACAGAGUGCGUGCUGAACAA |
| | AGCAACUAAUGUAGCUCAUUGGACAACCCCAUCACUGAAAUGCAUUAGAGAUCCAGCUC |
| | UGGUGCAUCAAAGACCAGCACCACCAAGUACCGUAACAACCGCAGGGGUGACCCCUCAG |
| | CCUGAGUCCCUAUCUCCCUCCGGCAAGGAGCCAGCAGCAUCUUCACCUAGCUCCAAUAA |
| | CACCGCAGCUACCACUGCCGCCAUAGUCCCCGGAAGCCAGCUGAUGCCUAGUAAAUCUC |
| | CCUCAACAGGUACCACCGAAAUUUCUAGCCAUGAGUCCUCGCACGGCACCCCGUCACAG |
| | ACUACAGCUAAAAACUGGGAGCUAACGGCUUCGGCAUCCCACCAACCUCCAGGCGUUUA |
| | UCCCCAAGGUCACUCCGACACUACUGUGGCGAUUAGCACAAGUACCGUCCUUCUGUGUG |
| | GACUGAGUGCAGUUUCAUUGCUGGCCUGUUAUCUGAAAUCUCGCCAGACCCCUCCCCUC |
| | GCCAGUGUUGAGAUGGAAGCCAUGGAAGCACUUCCUGUGACUUGGGGAACAUCCUCGAG |
| | GGACGAGGACCUCGAGAACUGCUCUCACCACCUG |
| | Full-Length IL15R mRNA ORF #1. Signal peptide is |
| | italicized, the sushi domain of the wild-type IL15Rα is |
| | double underlined. |
| 15 | 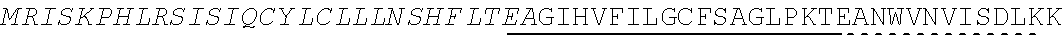 |
| | 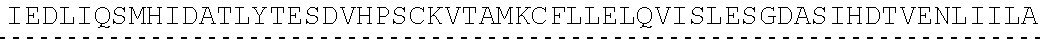 |
| | 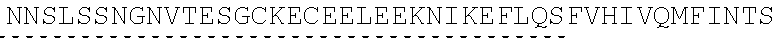 |
| | Full-Length IL15 Amino Acid Sequence (Wild Type). The |
| | signal peptide is italized. The propetide is solid line |
| | underlined. Mature IL15 is dot underlined. |
| 16 | AUGCGCAUCAGCAAACCUCAUUUACGGAGUAUCAGCAUCCAGUGCUAUCUCUGCCUGCU |
| | UCUGAACAGUCAUUUUCUGACUGAGGCGGGCAUUCAUGUCUUUAUUUUAGGCUGCUUUU |
| | CCGCAGGUCUGCCCAAAACAGAAGCAAAUUGGGUGAACGUGAUCAGCGACCUGAAGAAG |
| | AUUGAGGAUCUAAUUCAAAGCAUGCAUAUUGAUGCCACACUCUACACCGAAUCCGACGU |
| | GCACCCUUCGUGUAAAGUGACUGCAAUGAAGUGUUUCUUACUGGAACUGCAGGUGAUCA |
| | GUCUGGAGUCCGGGGAUGCAUCAAUCCACGACACAGUGGAAAACCUGAUUAUCCUGGCA |
| | AACAAUUCCCUGAGCAGUAAUGGCAAUGUCACGGAGAGCGGAUGUAAGGAGUGUGAGGA |
| | AUUAGAGGAAAAGAAUAUCAAGGAAUUCCUUCAGUCCUUUGUGCACAUCGUACAGAUGU |
| | UUAUUAAUACAUCC |
| | Full-length IL15 Nucleotide Sequence (Wild Type) |
| 17 | 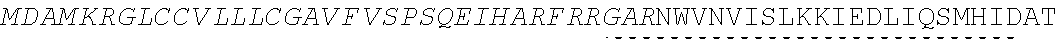 |
| | 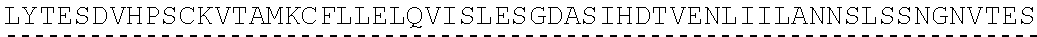 |
| | 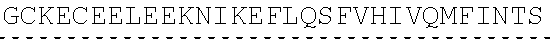 |
| | tPA-IL15 Amino acid Sequence. Signal peptide and propeptide |
| | from tPA is italicized; the mature IL15 peptide has a |
| | dotted underline |
| 18 | METDTLLLWVLLLWVPGSTGEPKSCDKTHTCPPCPAPELLGGPSVFLFPPKPKDTLMIS |
| | RTPEVTCVVVAVSHEDPEVKFNWYVDGVEVHNAKTKPREEQYNSTYRVVSVLTVLHQDW |
| | LNGKEYKCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSCSVMHEA |
| | LHNHYTQKSLSLSPCKITCPPPMSVEHADIWVKSYSLYSRERYICNSGFKRKAGTSSLT |
| | 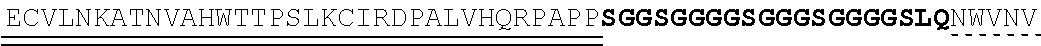 |
| | 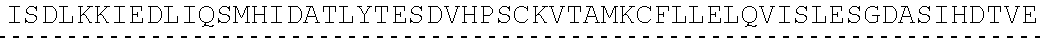 |
| | 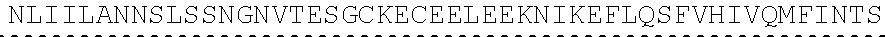 |
| | Full Fc-IL15R-Linker-IL15 Amino acid Sequence. The signal |
| | peptide is italicized and the mature IL15 is represented by |
| | a dotted underline. |
| 19 | AUGGAUGCAAUGAAGAGAGGGCUCUGCUGUGUGCUGCUGCUGUGUGGAGCAGUCUUCGU |
| | UUCGCCCAGCCAGGAAAUCCAUGCCCGAUUCAGAAGAGGAGCCAGAAACUGGGUGAACG |
| | UGAUCUCGGACCUGAAGAAGAUCGAGGACCUCAUCCAGUCGAUGCACAUCGACGCGACG |
| | CUGUACACGGAGUCGGACGUCCACCCGUCGUGCAAGGUCACGGCGAUGAAGUGCUUCCU |
| | CCUGGAGCUCCAAGUCAUCUCGCUCGAGUCGGGGGACGCGUCGAUCCACGACACGGUGG |
| | AGAACCUGAUCAUCCUGGCGAACAACUCGCUGUCGUCGAACGGGAACGUCACGGAGUCG |
| | GGCUGCAAGGAGUGCGAGGAGCUGGAGGAGAAGAACAUCAAGGAGUUCCUGCAGUCGUU |
| | CGUGCACAUCGUCCAGAUGUUCAUCAACACGUCG |
| | HS IL15opt-tPA6 mRNA ORF #1 |
| 20 | AUGGACGCCAUGAAGCGCGGCCUGUGCUGCGUGCUGCUGCUGUGCGGCGCCGUGUUCGU |
| | GAGCCCCUCCCAGGAGAUCCACGCAAGGUUCAGGAGGGGCGCCAGAAACUGGGUGAACG |
| | UGAUCUCUGAUCUGAAGAAGAUCGAGGAUCUCAUACAGAGCAUGCACAUCGACGCAACC |
| | CUGUACACCGAGAGCGACGUGCACCCUAGCUGCAAGGUGACAGCUAUGAAGUGCUUCCU |
| | GCUCGAGCUGCAGGUGAUCAGCCUGGAGAGCGGCGACGCCUCCAUUCACGACACCGUGG |
| | AGAACCUGAUCAUCCUGGCCAAUAACAGCCUGAGCAGCAACGGCAACGUGACAGAAAGC |
| | GGCUGUAAGGAGUGCGAGGAGCUGGAGGAGAAGAACAUCAAAGAGUUUCUGCAGAGCUU |
| | CGUGCACAUUGUGCAGAUGUUCAUCAACACCAGC |
| | Hs IL15opt-tPA6 mRNA ORF #3 |
| 21 | AUGGCACCAAGACGCGCCAGGGGCUGUCGCACCCUCGGACUUCCUGCACUCUUGCUUUU |
| | GCUCCUCCUUAGACCACCUGCAACCAGAGGGAUAACCUGUCCACCUCCAAUGAGCGUCG |
| | AGCACGCAGACAUUUGGGUGAAAUCAUACAGUCUGUACAGUAGAGAGCGGUACAUCUGC |
| | AACAGUGGGUUUAAGAGGAAGGCAGGCACUUCAUCUCUGACAGAGUGCGUGCUGAACAA |
| | AGCAACUAACGUAGCUCAUUGGACAACCCCAUCACUGAAGUGCAUUAGAGAUCCAGCUC |
| | UGGUGCAUCAAAGACCAGCACCACCAAGUACCGUAACAACCGCAGGGGUGACCCCUCAG |
| | CCUGAGUCCCUAUCUCCCUCCGGCAAGGAGCCAGCAGCAUCUUCACCUAGCUCCAAUAA |
| | CACCGCAGCUACCACUGCCGCCAUAGUCCCCGGAAGCCAGCUGAUGCCUAGUAAAUCUC |
| | CCUCAACAGGUACCACCGAAAUUUCUAGCCACGAGUCCUCGCACGGCACCCCGUCACAG |
| | ACUACAGCUAAGAACUGGGAGCUAACGGCUUCGGCAUCCCACCAACCUCCAGGCGUUUA |
| | UCCCCAAGGUCACUCCGACACUACUGUGGCGAUUAGCACAAGUACCGUCCUUCUGUGUG |
| | GACUGAGUGCAGUUUCAUUGCUGGCCUGUUAUCUGAAAUCUCGCCAGACCCCUCCCCUC |
| | GCCAGUGUUGAGAUGGAAGCCAUGGAAGCACUUCCUGUGACUUGGGGAACAUCCUCGAG |
| | GGACGAGGACCUCGAGAACUGCUCUCACCACCUG |
| | Hs IL15Ra mRNA ORF #2 |
| 22 | AUGGCCCCUAGACGGGCCAGGGGCUGCAGGACACUGGGCCUGCCCGCCCUGCUGCUCCU |
| | GCUGUUGCUCAGACCUCCCGCCACCAGAGGAAUCACCUGCCCUCCUCCCAUGAGCGUGG |
| | AGCACGCCGACAUCUGGGUGAAGUCCUACAGCCUGUACAGCCGGGAGAGAUACAUCUGU |
| | AACUCCGGCUUCAAGAGAAAGGCCGGCACCAGCUCCCUGACCGAGUGUGUGCUGAACAA |
| | GGCCACCAACGUGGCCCACUGGACCACACCCAGCCUGAAGUGCAUCAGAGAUCCCGCUC |
| | UGGUGCACCAAAGACCUGCUCCUCCUAGCACCGUGACCACAGCCGGCGUGACACCUCAA |
| | CCGGAAAGCCUGAGCCCCUCCGGCAAGGAGCCGGCCGCCUCCUCACCCAGCAGCAACAA |
| | CACCGCCGCCACAACCGCCGCUAUCGUGCCCGGCAGCCAGCUGAUGCCCAGCAAGAGCC |
| | CCAGCACAGGAACAACCGAGAUCAGCUCUCACGAAUCCAGCCACGGCACCCCUAGCCAG |
| | ACAACCGCUAAGAACUGGGAGCUGACAGCCAGCGCUAGCCACCAGCCACCCGGGGUGUA |
| | CCCACAGGGCCACAGCGACACCACCGUGGCCAUCAGCACCAGCACUGUGCUGCUGUGCG |
| | GGCUCAGCGCAGUGAGCCUGCUGGCUUGCUACCUUAAGUCCAGACAGACCCCUCCCCUG |
| | GCUUCCGUCGAAAUGGAGGCCAUGGAGGCUCUGCCUGUGACCUGGGGCACAAGCAGCAG |
| | GGACGAGGAUCUGGAGAACUGCUCUCACCACCUG |
| | Hs IL15Ra mRNA ORF #3 |
| 23 | MDWTWILFLVAAATRVHSNWVNVISDLKKIEDLIQSMHIDATLYTESDVHPSCKVTAMK |
| | CFLLELQVISLESGDASIHDTVENLIILANNSLSSNGNVTESGCKECEELEEKNIKEFL |
| | QSFVHIVQMFINTSSGGGSGGGGSGGGGSGGGGSGGGSLQITCPPPMSVEHADIWVKSY |
| | SLYSRERYICNSGFKRKAGTSSLTECVLNKATNVAHWTTPSLKCIRDPALVHQRPAPPS |
| | TVTTAGVTPQPESLSPSGKEPAASSPSSNNTAATTAAIVPGSQLMPSKSPSTGTTEISS |
| | HESSHGTPSQTTAKNWELTASASHQPPGVYPQGHSDTTVAISTSTVLLCGLSAVSLLAC |
| | 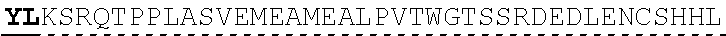 |
| | Hs IL15opt-IgEsp_IL15Ra WT (signal peptide is italic; IL-15 |
| | is underlined; linker is bold; sushi domain is underlined |
| | and italic; IL-15Ra is double underlined; transmembrane |
| | domain is bold and underlined; intracellular domain is |
| | dotted underlined) |
| 24 | AUGGACUGGACCUGGAUCCUGUUCCUGGUGGCCGCCGCCACCAGAGUGCACAGCAACUG |
| | GGUGAACGUGAUCUCGGACCUGAAGAAGAUCGAGGACCUCAUCCAGUCGAUGCACAUCG |
| | ACGCGACGCUGUACACGGAGUCGGACGUCCACCCGUCGUGCAAGGUCACGGCGAUGAAG |
| | UGCUUCCUCCUGGAGCUCCAAGUCAUCUCGCUCGAGUCGGGCGACGCGUCGAUCCACGA |
| | CACGGUGGAGAACCUGAUCAUCCUGGCGAACAACUCGCUGUCGUCGAACGGGAACGUCA |
| | CGGAGUCGGGCUGCAAGGAGUGCGAGGAGCUGGAGGAGAAGAACAUCAAGGAGUUCCUG |
| | CAGUCGUUCGUGCACAUCGUCCAGAUGUUCAUCAACACGUCGUCUGGUGGCGGAUCAGG |
| | UGGUGGCGGAUCUGGCGGUGGUGGAAGUGGAGGUGGCGGGUCUGGCGGAGGUUCACUGC |
| | AGAUAACCUGUCCACCUCCAAUGAGCGUCGAGCACGCAGACAUUUGGGUGAAAUCAUAC |
| | AGUCUGUACAGUAGAGAGCGGUACAUCUGCAACAGUGGGUUUAAGAGAAAGGCAGGCAC |
| | UUCAUCUCUGACAGAGUGCGUGCUGAACAAAGCAACUAAUGUAGCUCAUUGGACAACCC |
| | CAUCACUGAAAUGCAUUAGAGAUCCAGCUCUGGUGCAUCAAAGACCAGCACCACCAAGU |
| | ACCGUAACAACCGCAGGGGUGACCCCUCAGCCUGAGUCCCUAUCUCCCUCCGGCAAGGA |
| | GCCAGCAGCAUCUUCACCUAGCUCCAAUAACACCGCAGCUACCACUGCCGCCAUAGUCC |
| | CCGGAAGCCAGCUGAUGCCUAGUAAAUCUCCCUCAACAGGUACCACCGAAAUUUCUAGC |
| | CAUGAGUCCUCGCACGGCACCCCGUCACAGACUACAGCUAAGAACUGGGAGCUAACGGC |
| | UUCGGCAUCCCACCAACCUCCAGGCGUUUAUCCCCAAGGUCACUCCGACACUACUGUGG |
| | CGAUUAGCACAAGUACCGUCCUUCUGUGUGGACUGAGUGCAGUUUCAUUGCUGGCCUGU |
| | UAUCUGAAAUCUCGCCAGACCCCUCCCCUCGCCAGUGUUGAGAUGGAAGCCAUGGAAGC |
| | ACUUCCUGUGACUUGGGGAACAUCCUCGAGGGACGAGGACCUCGAGAACUGCUCUCACC |
| | ACCUG |
| | Nucleotide Sequence #1: Hs IL15opt-IgEsp_IL15Ra |
| 25 | AUGGACUGGACCUGGAUCCUGUUCCUGGUGGCUGCCGCCACCAGGGUGCACAGCAACUG |
| | GGUGAACGUGAUCUCGGACCUGAAGAAGAUCGAGGACCUCAUCCAGUCGAUGCACAUCG |
| | ACGCGACGCUGUACACGGAGUCGGACGUCCACCCGUCGUGCAAGGUCACGGCGAUGAAG |
| | UGCUUCCUCCUGGAGCUCCAAGUCAUCUCGCUCGAGUCGGGCGACGCGUCGAUCCACGA |
| | CACGGUGGAGAACCUGAUCAUCCUGGCGAACAACUCGCUGUCGUCGAACGGGAACGUCA |
| | CGGAGUCGGGCUGCAAGGAGUGCGAGGAGCUGGAGGAGAAGAACAUCAAGGAGUUCCUG |
| | CAGUCGUUCGUGCACAUCGUCCAGAUGUUCAUCAACACGUCGAGCGGCGGCGGUAGUGG |
| | AGGAGGUGGCUCCGGUGGAGGCGGCAGCGGUGGCGGUGGAAGUGGCGGCGGCAGCCUGC |
| | AGAUCACCUGUCCUCCUCCCAUGAGCGUGGAGCACGCCGACAUCUGGGUGAAGAGCUAC |
| | AGCCUGUACAGCCGGGAGCGGUACAUCUGCAACAGCGGCUUCAAGCGGAAGGCCGGCAC |
| | AAGCAGCCUGACCGAGUGCGUGCUGAACAAGGCCACCAACGUGGCCCACUGGACCACCC |
| | CAAGCCUGAAGUGCAUCCGGGACCCCGCCCUGGUGCAUCAGCGGCCCGCCCCACCUAGC |
| | ACCGUGACCACCGCCGGCGUGACACCCCAGCCCGAGAGCCUGAGCCCCAGCGGCAAGGA |
| | GCCCGCUGCCAGCUCCCCAAGCAGCAACAACACCGCCGCCACAACCGCCGCCAUCGUGC |
| | CCGGCAGCCAGCUGAUGCCCAGCAAGAGCCCCAGCACCGGCACCACCGAGAUCAGCAGC |
| | CACGAGAGCAGCCACGGCACACCCAGCCAGACCACCGCCAAGAACUGGGAGCUGACCGC |
| | CAGCGCCAGCCACCAGCCUCCCGGCGUGUAUCCCCAGGGCCACAGCGACACCACCGUGG |
| | CCAUCAGCACCAGCACCGUGCUGCUGUGCGGCCUGAGCGCCGUGAGCCUGCUGGCCUGC |
| | UACCUGAAGAGCCGGCAGACCCCACCCCUGGCCAGCGUGGAGAUGGAGGCCAUGGAGGC |
| | CCUGCCCGUGACCUGGGGCACCAGCAGCCGGGACGAGGACCUGGAGAACUGCAGCCACC |
| | ACCUG |
| | Nucleotide Sequence #2: Hs IL15opt-IgEsp_IL15Ra |
| 26 | AUGGACUGGACCUGGAUCCUGUUUCUCGUUGCCGCCGCUACCAGAGUGCACAGCAACUG |
| | GGUGAACGUGAUCAGCGACCUGAAGAAGAUCGAGGACCUGAUCCAGAGCAUGCACAUCG |
| | ACGCCACCCUGUACACCGAGAGCGACGUGCACCCCAGCUGCAAGGUGACCGCCAUGAAG |
| | UGCUUCCUGCUGGAGCUGCAGGUGAUCAGCCUGGAGAGCGGCGACGCCAGCAUCCACGA |
| | CACCGUGGAGAACCUGAUCAUCCUGGCCAACAACAGCCUGAGCAGCAACGGCAACGUGA |
| | CCGAGAGCGGCUGCAAGGAGUGCGAGGAGCUGGAGGAGAAGAACAUCAAGGAGUUCCUG |
| | CAGAGCUUCGUGCACAUCGUGCAGAUGUUCAUCAACACCAGCAGUGGUGGCGGAAGUGG |
| | CGGCGGAGGCAGUGGAGGCGGCGGCUCAGGAGGCGGUGGCUCAGGCGGAGGUAGCCUGC |
| | AGAUCACCUGCCCACCUCCCAUGAGCGUGGAGCACGCCGACAUCUGGGUGAAGAGCUAC |
| | AGCCUGUACAGCCGGGAGCGGUACAUCUGCAACAGCGGCUUCAAGCGGAAGGCCGGCAC |
| | CAGCAGCCUGACCGAGUGCGUGCUGAACAAGGCCACCAACGUGGCCCACUGGACAACCC |
| | CUAGCCUGAAGUGCAUUAGAGACCCGGCCCUGGUGCAUCAACGGCCCGCCCCACCUAGC |
| | ACCGUGACCACCGCCGGCGUGACUCCCCAGCCCGAGAGCCUGAGCCCCAGCGGCAAGGA |
| | ACCCGCCGCCAGUAGCCCCAGCAGCAACAACACAGCCGCCACUACCGCCGCCAUCGUGC |
| | CCGGCAGCCAGCUGAUGCCCAGCAAGAGCCCCAGCACCGGCACCACCGAGAUCAGCAGC |
| | CACGAGAGCAGCCACGGCACCCCUAGCCAGACCACCGCCAAGAACUGGGAGCUGACCGC |
| | CAGCGCCAGCCACCAACCACCAGGCGUGUACCCUCAGGGCCACAGCGACACCACCGUGG |
| | CCAUCAGCACCAGCACCGUGCUGCUGUGCGGCCUGAGCGCCGUGAGCCUGCUGGCCUGC |
| | UACCUGAAGAGCCGGCAGACCCCACCCCUGGCCAGCGUGGAGAUGGAGGCCAUGGAGGC |
| | CCUGCCCGUGACCUGGGGCACCAGCUCUCGGGACGAGGACCUGGAGAACUGCAGCCACC |
| | ACCUG |
| | Nucleotide Sequence #3: Hs IL15opt-IgEsp_IL15Ra |
| 27 | MDAMKRGLCCVLLLCGAVFVSPSQEIHARFRRGARNWVNVISDLKKIEDLIQSMHIDAT |
| | LYTESDVHPSCKVTAMKCFLLELQVISLESGDASIHDTVENLIILANNSLSSNGNVTES |
| | GCKECEELEEKNIKEFLQSFVHIVQMFINTSSGGGSGGGGSGGGGSGGGGSGGGSLQIT |
| | CPPPMSVEHADIWVKSYSLYSRERYICNSGFKRKAGTSSLTECVLNKATNVAHWTTPSL |
| | KCIRDPALVHQRPAPPSTVTTAGVTPQPESLSPSGKEPAASSPSSNNTAATTAAIVPGS |
| | QLMPSKSPSTGTTEISSHESSHGTPSQTTAKNWELTASASHQPPGVYPQGHSDTTVAIS |
| | 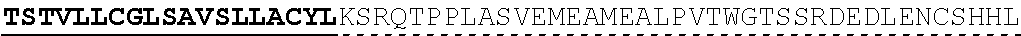 |
| | Hs IL15opt-tPA6_IL15Ra WT (signal peptide is italic; IL-15 |
| | is underlined; linker is bold; sushi domain is underlined |
| | and italic; IL-15Ra is double underlined; transmembrane |
| | domain is bold and underlined; intracellular domain is |
| | dotted underlined) |
| 28 | AUGGAUGCAAUGAAGAGAGGGCUCUGCUGUGUGCUGCUGCUGUGUGGAGCAGUCUUCGU |
| | UUCGCCCAGCCAGGAAAUCCAUGCCCGAUUCAGAAGAGGAGCCAGAAACUGGGUGAACG |
| | UGAUCUCGGACCUGAAGAAGAUCGAGGACCUCAUCCAGUCGAUGCACAUCGACGCGACG |
| | CUGUACACGGAGUCGGACGUCCACCCGUCGUGCAAGGUCACGGCGAUGAAGUGCUUCCU |
| | CCUGGAGCUCCAAGUCAUCUCGCUCGAGUCGGGCGACGCGUCGAUCCACGACACGGUGG |
| | AGAACCUGAUCAUCCUGGCGAACAACUCGCUGUCGUCGAACGGGAACGUCACGGAGUCG |
| | GGCUGCAAGGAGUGCGAGGAGCUGGAGGAGAAGAACAUCAAGGAGUUCCUGCAGUCGUU |
| | CGUGCACAUCGUCCAGAUGUUCAUCAACACGUCGUCUGGUGGCGGAUCAGGUGGUGGCG |
| | GAUCUGGCGGUGGUGGAAGUGGAGGUGGCGGGUCUGGCGGAGGUUCACUGCAGAUAACC |
| | UGUCCACCUCCAAUGAGCGUCGAGCACGCAGACAUUUGGGUGAAAUCAUACAGUCUGUA |
| | CAGUAGAGAGCGGUACAUCUGCAACAGUGGGUUUAAGAGAAAGGCAGGCACUUCAUCUC |
| | UGACAGAGUGCGUGCUGAACAAAGCAACUAAUGUAGCUCAUUGGACAACCCCAUCACUG |
| | AAAUGCAUUAGAGAUCCAGCUCUGGUGCAUCAAAGACCAGCACCACCAAGUACCGUAAC |
| | AACCGCAGGGGUGACCCCUCAGCCUGAGUCCCUAUCUCCCUCCGGCAAGGAGCCAGCAG |
| | CAUCUUCACCUAGCUCCAAUAACACCGCAGCUACCACUGCCGCCAUAGUCCCCGGAAGC |
| | CAGCUGAUGCCUAGUAAAUCUCCCUCAACAGGUACCACCGAAAUUUCUAGCCAUGAGUC |
| | CUCGCACGGCACCCCGUCACAGACUACAGCUAAGAACUGGGAGCUAACGGCUUCGGCAU |
| | CCCACCAACCUCCAGGCGUUUAUCCCCAAGGUCACUCCGACACUACUGUGGCGAUUAGC |
| | ACAAGUACCGUCCUUCUGUGUGGACUGAGUGCAGUUUCAUUGCUGGCCUGUUAUCUGAA |
| | AUCUCGCCAGACCCCUCCCCUCGCCAGUGUUGAGAUGGAAGCCAUGGAAGCACUUCCUG |
| | UGACUUGGGGAACAUCCUCGAGGGACGAGGACCUCGAGAACUGCUCUCACCACCUG |
| | Nucleotide Sequence #1: Hs IL15opt-tPA6_IL15Ra WT |
| 29 | AUGGAUGCAAUGAAGAGAGGGCUCUGCUGUGUGCUGCUGCUGUGUGGAGCAGUCUUCGU |
| | UUCGCCCAGCCAGGAAAUCCAUGCCCGAUUCAGAAGAGGAGCCAGAAACUGGGUGAACG |
| | UGAUCUCGGACCUGAAGAAGAUCGAGGACCUCAUCCAGUCGAUGCACAUCGACGCGACG |
| | CUGUACACGGAGUCGGACGUCCACCCGUCGUGCAAGGUCACGGCGAUGAAGUGCUUCCU |
| | CCUGGAGCUCCAAGUCAUCUCGCUCGAGUCGGGCGACGCGUCGAUCCACGACACGGUGG |
| | AGAACCUGAUCAUCCUGGCGAACAACUCGCUGUCGUCGAACGGGAACGUCACGGAGUCG |
| | GGCUGCAAGGAGUGCGAGGAGCUGGAGGAGAAGAACAUCAAGGAGUUCCUGCAGUCGUU |
| | CGUGCACAUCGUCCAGAUGUUCAUCAACACGUCGAGCGGCGGCGGUAGUGGAGGAGGUG |
| | GCUCCGGUGGAGGCGGCAGCGGUGGCGGUGGAAGUGGCGGCGGCAGCCUGCAGAUCACC |
| | UGUCCUCCUCCCAUGAGCGUGGAGCACGCCGACAUCUGGGUGAAGAGCUACAGCCUGUA |
| | CAGCCGGGAGCGGUACAUCUGCAACAGCGGCUUCAAGCGGAAGGCCGGCACAAGCAGCC |
| | UGACCGAGUGCGUGCUGAACAAGGCCACCAACGUGGCCCACUGGACCACCCCAAGCCUG |
| | AAGUGCAUCCGGGACCCCGCCCUGGUGCAUCAGCGGCCCGCCCCACCUAGCACCGUGAC |
| | CACCGCCGGCGUGACACCCCAGCCCGAGAGCCUGAGCCCCAGCGGCAAGGAGCCCGCUG |
| | CCAGCUCCCCAAGCAGCAACAACACCGCCGCCACAACCGCCGCCAUCGUGCCCGGCAGC |
| | CAGCUGAUGCCCAGCAAGAGCCCCAGCACCGGCACCACCGAGAUCAGCAGCCACGAGAG |
| | CAGCCACGGCACACCCAGCCAGACCACCGCCAAGAACUGGGAGCUGACCGCCAGCGCCA |
| | GCCACCAGCCUCCCGGCGUGUAUCCCCAGGGCCACAGCGACACCACCGUGGCCAUCAGC |
| | ACCAGCACCGUGCUGCUGUGCGGCCUGAGCGCCGUGAGCCUGCUGGCCUGCUACCUGAA |
| | GAGCCGGCAGACCCCACCCCUGGCCAGCGUGGAGAUGGAGGCCAUGGAGGCCCUGCCCG |
| | UGACCUGGGGCACCAGCAGCCGGGACGAGGACCUGGAGAACUGCAGCCACCACCUG |
| | Nucleotide Sequence #2: Hs IL15-tPA6_IL15Ra-opt |
| 30 | AUGGACGCCAUGAAGCGGGGCCUGUGCUGCGUGCUGCUGCUGUGCGGCGCCGUGUUCGU |
| | GAGCCCCAGCCAGGAGAUCCACGCCCGUUUCCGACGGGGCGCCAGAAACUGGGUGAACG |
| | UGAUCAGCGACCUGAAGAAGAUCGAGGACCUGAUCCAGAGCAUGCACAUCGACGCCACC |
| | CUGUACACCGAGAGCGACGUGCACCCCAGCUGCAAGGUGACCGCCAUGAAGUGCUUCCU |
| | GCUGGAGCUGCAGGUGAUCAGCCUGGAGAGCGGCGACGCCAGCAUCCACGACACCGUGG |
| | AGAACCUGAUCAUCCUGGCCAACAACAGCCUGAGCAGCAACGGCAACGUGACCGAGAGC |
| | GGCUGCAAGGAGUGCGAGGAGCUGGAGGAGAAGAACAUCAAGGAGUUCCUGCAGAGCUU |
| | CGUGCACAUCGUGCAGAUGUUCAUCAACACCAGCUCUGGCGGCGGCUCAGGAGGAGGAG |
| | GCAGCGGAGGAGGCGGUAGCGGCGGAGGUGGAAGCGGCGGCGGAAGCCUGCAGAUCACC |
| | UGUCCGCCUCCCAUGAGCGUGGAGCACGCCGACAUCUGGGUGAAGAGCUACAGCCUGUA |
| | CAGCCGGGAGCGGUACAUCUGCAACAGCGGCUUCAAGCGGAAGGCCGGUACCAGCAGUC |
| | UGACCGAGUGCGUGCUGAACAAGGCCACCAACGUGGCCCACUGGACCACCCCAAGCCUG |
| | AAGUGCAUCCGCGACCCAGCCCUGGUGCAUCAGCGGCCCGCUCCUCCAAGCACCGUGAC |
| | CACCGCUGGCGUGACCCCACAGCCCGAGAGCCUGUCCCCAAGCGGCAAGGAGCCCGCCG |
| | CUAGCAGCCCCAGCAGCAACAACACAGCCGCCACCACCGCUGCCAUCGUGCCCGGCAGC |
| | CAGCUGAUGCCCAGCAAGAGCCCCAGCACCGGCACCACCGAGAUCAGCAGCCACGAGAG |
| | CAGCCACGGCACCCCUAGCCAGACCACCGCCAAGAACUGGGAGCUGACCGCCAGCGCCA |
| | GCCACCAGCCACCUGGCGUGUAUCCCCAGGGCCACAGCGACACCACCGUGGCCAUCAGC |
| | ACCAGCACCGUGCUGCUGUGUGGCCUGAGCGCCGUGAGCCUGCUGGCCUGCUACCUGAA |
| | GUCCCGCCAAACCCCGCCACUGGCCAGCGUGGAGAUGGAGGCCAUGGAGGCCCUGCCCG |
| | UGACCUGGGGCACCAGCAGCCGGGACGAGGACCUGGAGAACUGCAGCCACCACCUG |
| | Nucleotide Sequence #3: Hs IL15-15Ra_tPA6_fullopt |
| 31 | METDTLLLWVLLLWVPGSTGDYKDDDDKITCPPPMSVEHADIWVKSYSLYSRERYICNS |
| | GFKRKAGTSSLTECVLNKATNVAHWTTPSLKCIRDPALVHQRPAPPSGGSGGGGSGGGS |
| | 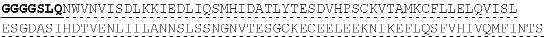 |
| | Hs IL-15 RLI (signal peptide is italic; epitope tag is |
| | bold; sushi domain is underlined and italic; linker is |
| | underlined and bold; IL-15 is dotted underlined) |
| 32 | AUGGAAACCGACACUCUGCUGCUUUGGGUGCUGCUGCUCUGGGUGCCCGGAUCCACCGG |
| | AGAUUACAAGGACGAUGACGACAAGAUCACCUGUCCACCUCCCAUGUCCGUGGAACACG |
| | CCGACAUCUGGGUCAAAAGCUACAGCCUGUACUCCCGGGAAAGAUACAUUUGUAACAGC |
| | GGGUUUAAGAGGAAGGCCGGCACCUCCUCGUUGACCGAAUGCGUGCUGAACAAGGCUAC |
| | CAACGUGGCCCAUUGGACUACCCCGUCCCUGAAGUGCAUUCGCGAUCCUGCCCUCGUGC |
| | ACCAACGGCCUGCGCCGCCGUCCGGAGGGUCUGGCGGUGGUGGAUCGGGAGGCGGUUCA |
| | GGAGGCGGAGGGUCGCUGCAGAACUGGGUGAACGUGAUCUCCGACCUUAAGAAGAUCGA |
| | GGACCUGAUCCAGUCCAUGCACAUCGACGCAACCCUGUAUACUGAGUCAGACGUGCACC |
| | CCUCCUGCAAAGUCACAGCGAUGAAGUGCUUCCUGCUGGAACUCCAGGUCAUCUCCUUG |
| | GAAUCGGGAGAUGCCUCCAUUCACGACACUGUCGAGAACCUCAUUAUUCUGGCCAACAA |
| | CAGCCUGUCCAGCAAUGGCAACGUGACGGAGUCAGGCUGCAAGGAGUGCGAGGAACUCG |
| | AAGAGAAGAAUAUCAAGGAGUUCCUGCAGUCGUUCGUGCAUAUCGUGCAAAUGUUCAUC |
| | AACACCAGC |
| | Hs IL15-RLI mRNA open reading frame |
| 33 | IWELKKDVYVVELDWYPDAPGEMVVLTCDTPEEDGITWTLDQSSEVLGSGKTLTIQVKE |
| | FGDAGQYTCHKGGEVLSHSLLLLHKKEDGIWSTDILKDQKEPKNKTFLRCEAKNYSGRF |
| | TCWWLTTISTDLTFSVKSSRGSSDPQGVTCGAATLSAERVRGDNKEYEYSVECQEDSAC |
| | PAAEESLPIEVMVDAVHKLKYENYTSSFFIRDIIKPDPPKNLQLKPLKNSRQVEVSWEY |
| | PDTWSTPHSYFSLTFCVQVQGKSKREKKDRVFTDKTSATVICRKNASISVRAQDRYYSS |
| | SWSEWASVPCS |
| | Wildtype IL12B without signal amino acids |
| 34 | AUAUGGGAACUGAAGAAAGAUGUUUAUGUCGUAGAAUUGGAUUGGUAUCCGGAUGCCCC |
| | UGGAGAAAUGGUGGUCCUCACCUGUGACACCCCUGAAGAAGAUGGUAUCACCUGGACCU |
| | UGGACCAGAGCAGUGAGGUCUUAGGCUCUGGCAAAACCCUGACCAUCCAAGUCAAAGAG |
| | UUUGGAGAUGCUGGCCAGUACACCUGUCACAAAGGAGGCGAGGUUCUAAGCCAUUCGCU |
| | CCUGCUGCUUCACAAAAAGGAAGAUGGAAUUUGGUCCACUGAUAUUUUAAAGGACCAGA |
| | AAGAACCCAAAAAUAAGACCUUUCUAAGAUGCGAGGCCAAGAAUUAUUCUGGACGUUUC |
| | ACCUGCUGGUGGCUGACGACAAUCAGUACUGAUUUGACAUUCAGUGUCAAAAGCAGCAG |
| | AGGCUCUUCUGACCCCCAAGGGGUGACGUGCGGAGCUGCUACACUCUCUGCAGAGAGAG |
| | UCAGAGGGGACAACAAGGAGUAUGAGUACUCAGUGGAGUGCCAGGAGGACAGUGCCUGC |
| | CCAGCUGCUGAGGAGAGUCUGCCCAUUGAGGUCAUGGUGGAUGCCGUUCACAAGCUCAA |
| | GUAUGAAAACUACACCAGCAGCUUCUUCAUCAGGGACAUCAUCAAACCUGACCCACCCA |
| | AGAACUUGCAGCUGAAGCCAUUAAAGAAUUCUCGGCAGGUGGAGGUCAGCUGGGAGUAC |
| | CCUGACACCUGGAGUACUCCACAUUCCUACUUCUCCCUGACAUUCUGCGUUCAGGUCCA |
| | GGGCAAGAGCAAGAGAGAAAAGAAAGAUAGAGUCUUCACGGACAAGACCUCAGCCACGG |
| | UCAUCUGCCGCAAAAAUGCCAGCAUUAGCGUGCGGGCCCAGGACCGCUACUAUAGCUCA |
| | UCUUGGAGCGAAUGGGCAUCUGUGCCCUGCAGU |
| | Wildtype IL12B without signal nucleic acids |
| 35 | RNLPVATPDPGMFPCLHHSQNLLRAVSNMLQKARQTLEFYPCTSEEIDHEDITKDKTST |
| | VEACLPLELTKNESCLNSRETSFITNGSCLASRKTSFMMALCLSSIYEDLKMYQVEFKT |
| | MNAKLLMDPKRQIFLDQNMLAVIDELMQALNFNSETVPQKSSLEEPDFYKTKIKLCILL |
| | HAFRIRAVTIDRVMSYLNAS |
| | Wildtype IL12A without signal amino acids |
| 36 | AGAAACCUCCCCGUGGCCACUCCAGACCCAGGAAUGUUCCCAUGCCUUCACCACUCCCA |
| | AAACCUGCUGAGGGCCGUCAGCAACAUGCUCCAGAAGGCCAGACAAACUCUAGAAUUUU |
| | ACCCUUGCACUUCUGAAGAGAUUGAUCAUGAAGAUAUCACAAAAGAUAAAACCAGCACA |
| | GUGGAGGCCUGUUUACCAUUGGAAUUAACCAAGAAUGAGAGUUGCCUAAAUUCCAGAGA |
| | GACCUCUUUCAUAACUAAUGGGAGUUGCCUGGCCUCCAGAAAGACCUCUUUUAUGAUGG |
| | CCCUGUGCCUUAGUAGUAUUUAUGAAGACUUGAAGAUGUACCAGGUGGAGUUCAAGACC |
| | AUGAAUGCAAAGCUUCUGAUGGAUCCUAAGAGGCAGAUCUUUCUAGAUCAAAACAUGCU |
| | GGCAGUUAUUGAUGAGCUGAUGCAGGCCCUGAAUUUCAACAGUGAGACUGUGCCACAAA |
| | AAUCCUCCCUUGAAGAACCGGAUUUUUAUAAAACUAAAAUCAAGCUCUGCAUACUUCUU |
| | CAUGCUUUCAGAAUUCGGGCAGUGACUAUUGAUAGAGUGAUGAGCUAUCUGAAUGCUUCC |
| | Wildtype IL12A without signal nucleic acids |
| 37 | MCHQQLVISWFSLVFLASPLVA |
| | Wildtype IL12B signal peptide amino acids |
| 38 | AUGUGUCACCAGCAGUUGGUCAUCUCUUGGUUUUCCCUGGUUUUUCUGGCAUCUCCCCU |
| | CGUGGCC |
| | Wildtype IL12B signal peptide nucleic acids |
| 39 | MCHQQLVISWFSLVFLASPLVAIWELKKDVYVVELDWYPDAPGEMVVLTCDTPEED |
| | GITWTLDQSSEVLGSGKTLTIQVKEFGDAGQYTCHKGGEVLSHSLLLLHKKEDGIWSTD |
| | ILKDQKEPKNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYTSSFFIRDII |
| | KPDPPKNLQLKPLKNSRQVEVSWEYPDTWSTPHSYFSLTFCVQVQGKSKREKKDRVFTD |
| | KTSATVICRKNASISVRAQDRYYSSSWSEWASVPCSGGGGGGSRNLPVATPDPGMFPCL |
| | HHSQNLLRAVSNMLQKARQTLEFYPCTSEEIDHEDITKDKTSTVEACLPLELTKNESCL |
| | NSRETSFITNGSCLASRKTSFMMALCLSSIYEDLKMYQVEFKTMNAKLLMDPKRQIFLD |
| | QNMLAVIDELMQALNFNSETVPQKSSLEEPDFYKTKIKLCILLHAFRIRAVTIDRVMSY |
| | LNAS |
| | Signal peptide-IL12B-linker-IL12A amino acid sequence #1 |
| 40 | MGCHQQLVISWFSLVFLASPLVAIWELKKDVYVVELDWYPDAPGEMWLTCDTPEE |
| | DGITWTLDQSSEVLGSGKTLTIQVKEFGDAGQYTCHKGGEVLSHSLLLLHKKEDGIWST |
| | DILKDQKEPKNKTFLRCEAKNYSGRFTCWWLTTISTDLTFSVKSSRGSSDPQGVTCGAA |
| | TLSAERVRGDNKEYEYSVECQEDSACPAAEESLPIEVMVDAVHKLKYENYTSSFFIRDI |
| | IKPDPPKNLQLKPLKNSRQVEVSWEYPDTWSTPHSYFSLTFCVQVQGKSKREKKDRVFT |
| | DKTSATVICRKNASISVRAQDRYYSSSWSEWASVPCSGGGGGGSRNLPVATPDPGMFPC |
| | LHHSQNLLRAVSNMLQKARQTLEFYPCTSEEIDHEDITKDKTSTVEACLPLELTKNESC |
| | LNSRETSFITNGSCLASRKTSFMMALCLSSIYEDLKMYQVEFKTMNAKLLMDPKRQIFL |
| | DQNMLAVIDELMQALNFNSETVPQKSSLEEPDFYKTKIKLCILLHAFRIRAVTIDRVMS |
| | YLNAS |
| | Signal peptide-IL12B-linker-IL12A amino acid sequence #2 |
| 41 | IYIWAPLAGTCGVLLLSLVITLYCY |
| | Human CD8A transmembrane domain |
| 42 | VVVISAILALVVLTIISLIILIMLW |
| | Human PDGF-RB transmembrane domain |
| 43 | LLPSWAITLISVNGIFVICCL |
| | Human CD80 Transmembrane domain |
| 44 | AUGUGUCCUCAGAAGCUAACCAUCUCCUGGUUUGCCAUCGUUUUGCUUGUGUCUCCACU |
| | CAUGGCCAUGUGGGAGCUCGAGAAAGACGUUUACGUUGUAGAGGUGGACUGGACUCCCG |
| | ACGCCCCAGGAGAAACAGUGAACCUCACCUGUGACACGCCUGAAGAAGAUGACAUCACC |
| | UGGACCUCAGACCAGAGACAUGGAGUCAUAGGCUCUGGAAAGACCCUGACCAUCACUGU |
| | CAAAGAGUUCCUAGAUGCUGGCCAGUACACCUGCCACAAAGGAGGCGAGACUCUGAGCC |
| | ACUCACAUCUGCUGCUCCACAAGAAGGAGAAUGGAAUUUGGUCCACCGAAAUCCUGAAG |
| | AACUUCAAGAAUAAGACUUUCCUGAAGUGUGAAGCACCAAAUUACUCCGGACGGUUCAC |
| | GUGCUCAUGGCUGGUGCAAAGAAACAUGGACUUGAAGUUCAACAUCAAGAGCAGUAGCA |
| | GUUCCCCUGACUCUCGGGCAGUGACAUGUGGAAUGGCGUCUCUGUCUGCAGAGAAGGUC |
| | ACACUGGACCAAAGGGACUAUGAGAAGUAUUCAGUGUCCUGCCAGGAGGAUGUCACCUG |
| | CCCAACUGCCGAGGAAACUCUGCCCAUUGAACUGGCGUUGGAAGCACGGCAGCAGAAUA |
| | AAUAUGAGAACUACAGCACCAGCUUCUUCAUCAGGGACAUCAUCAAACCAGACCCGCCC |
| | AAGAACUUGCAGAUGAAGCCUUUGAAGAACUCACAGGUGGAGGUCAGCUGGGAGUACCC |
| | AGACUCCUGGAGCACUCCCCAUUCCUACUUCUCCCUCAAGUUCUUUGUUCGAAUCCAGC |
| | GCAAGAAAGAGAAGAUGAAGGAGACAGAGGAGGGGUGUAACCAGAAAGGUGCGUUCCUC |
| | GUAGAGAAGACAUCUACCGAAGUCCAAUGCAAAGGCGGGAAUGUCUGCGUGCAAGCUCA |
| | GGAUCGCUAUUACAAUUCCUCAUGCAGCAAGUGGGCAUGUGUUCCCUGCAGGGUCCGAU |
| | CCGGAGGCGGAGGGUCUGGAGGAGGAGGUUCUGGAGGUGGUGGCAGUAGGGUCAUUCCA |
| | GUCUCUGGACCUGCAAGGUGUCUUAGCCAGUCCCGAAACCUGCUGAAGACCACAGACGA |
| | UAUGGUGAAGACGGCCAGAGAGAAACUGAAACAUUAUUCCUGCACAGCAGAGGACAUCG |
| | AUCAUGAAGAUAUUACACGGGACCAAACCAGCACAUUGAAGACCUGUUUACCACUGGAA |
| | CUACACAAGAACGAGAGUUGCCUGGCUACUAGAGAGACUUCUUCCACAACAAGAGGGAG |
| | CUGCCUGCCACCACAGAAGACGUCUUUGAUGAUGACCCUGUGCCUUGGUAGCAUCUACG |
| | AGGAUCUCAAGAUGUACCAGACAGAGUUCCAGGCCAUCAACGCAGCACUUCAGAAUCAC |
| | AACCAUCAGCAGAUCAUUUUAGACAAGGGCAUGCUGGUGGCCAUCGAUGAGCUGAUGCA |
| | AUCUCUGAAUCAUAAUGGCGAGACACUUCGCCAGAAACCUCCUGUGGGAGAAGCAGACC |
| | CUUACAGAGUGAAGAUGAAGCUCUGCAUCCUGCUUCACGCCUUCAGCACCCGCGUCGUC |
| | ACUAUUAACAGGGUGAUGGGCUAUCUGAGCUCCGCCUCUGGUGGCGGAUCAGGCGGCGG |
| | CGGCUCUGGCGGCGGUGGAAGCGGAGGUGGCGGGUCUGGCGGAGGUUCACUGCAGGUAG |
| | UAGUGAUCAGCGCCAUCCUGGCCCUGGUGGUGCUGACCGUGAUCUCAUUGAUCAUCUUG |
| | AUUAUGCUGUGGGGCGGAGGAGGCAGCGGGAAACCAAUUCCAAAUCCCCUCCUGGGGUU |
| | GGAUAGCACC |
| | mIL12AB-PTM_v5 miR122 Nucleotide Sequence |
| 45 | MCPQKLTISWFAIVLLVSPLMAMWELEKDVYVVEVDWTPDAPGETVNLTCDTPEEDDIT |
| | WTSDQRHGVIGSGKTLTITVKEFLDAGQYTCHKGGETLSHSHLLLHKKENGIWSTEILK |
| | NFKNKTFLKCEAPNYSGRFTCSWLVQRNMDLKFNIKSSSSSPDSRAVTCGMASLSAEKV |
| | TLDQRDYEKYSVSCQEDVTCPTAEETLPIELALEARQQNKYENYSTSFFIRDIIKPDPP |
| | KNLQMKPLKNSQVEVSWEYPDSWSTPHSYFSLKFFVRIQRKKEKMKETEEGCNQKGAFL |
| | 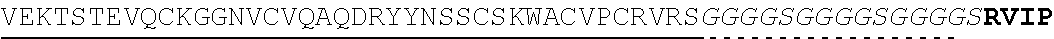 |
| | VSGPARCLSQSRNLLKTTDDMVKTAREKLKHYSCTAEDIDHEDITRDQTSTLKTCLPLE |
| | LHKNESCLATRETSSTTRGSCLPPQKTSLMMTLCLGSIYEDLKMYQTEFQAINAALQNH |
| | NHQQIILDKGMLVAIDELMQSLNHNGETLRQKPPVGEADPYRVKMKLCILLHAFSTRVV |
| | 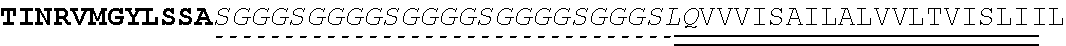 |
| | 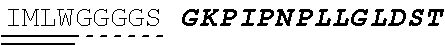 |
| | Italic: signal peptide; Underline: IL12B; Dashed underline |
| | and Italic: linker; Bold: IL12A; Double underline: |
| | Transmembrane domain; Bold and Italic: V5 tag |
| | mIL12AB-PTM_v5 miR122 Amino Acid Sequence |
| 46 | AUGUGCCACCAGCAGCUGGUGAUCAGCUGGUUCAGCCUGGUGUUCCUGGCCAGCCCCCU |
| | GGUGGCCAUCUGGGAGCUGAAGAAGGACGUGUACGUGGUGGAGUUGGAUUGGUACCCCG |
| | ACGCCCCCGGCGAGAUGGUGGUGCUGACCUGCGACACCCCCGAGGAGGACGGCAUCACC |
| | UGGACCCUGGACCAGAGCAGCGAGGUGCUGGGCAGCGGCAAGACCCUGACCAUCCAGGU |
| | GAAGGAGUUCGGCGACGCCGGCCAGUACACCUGCCACAAGGGCGGCGAGGUGCUGAGCC |
| | ACAGCCUGCUGCUGCUGCACAAGAAGGAGGACGGCAUCUGGAGCACCGACAUCCUGAAG |
| | GACCAGAAGGAGCCCAAGAACAAGACCUUCCUGAGAUGCGAGGCCAAGAACUACAGCGG |
| | CAGAUUCACCUGCUGGUGGCUGACCACCAUCAGCACCGACCUGACCUUCAGCGUGAAGA |
| | GCAGCAGAGGCAGCAGCGACCCCCAGGGCGUGACCUGCGGCGCCGCCACCCUGAGCGCC |
| | GAGAGAGUGAGAGGCGACAACAAGGAGUACGAGUACAGCGUGGAGUGCCAGGAAGAUAG |
| | CGCCUGCCCCGCCGCCGAGGAGAGCCUGCCCAUCGAGGUGAUGGUGGACGCCGUGCACA |
| | AGCUGAAGUACGAGAACUACACCAGCAGCUUCUUCAUCAGAGAUAUCAUCAAGCCCGAC |
| | CCCCCCAAGAACCUGCAGCUGAAGCCCCUGAAGAACAGCCGGCAGGUGGAGGUGAGCUG |
| | GGAGUACCCCGACACCUGGAGCACCCCCCACAGCUACUUCAGCCUGACCUUCUGCGUGC |
| | AGGUGCAGGGCAAGAGCAAGAGAGAGAAGAAAGAUAGAGUGUUCACCGACAAGACCAGC |
| | GCCACCGUGAUCUGCAGAAAGAACGCCAGCAUCAGCGUGAGAGCCCAAGAUAGAUACUA |
| | CAGCAGCAGCUGGAGCGAGUGGGCCAGCGUGCCCUGCAGCGGCGGCGGCGGCGGCGGCA |
| | GCAGAAACCUGCCCGUGGCCACCCCCGACCCCGGCAUGUUCCCCUGCCUGCACCACAGC |
| | CAGAACCUGCUGAGAGCCGUGAGCAACAUGCUGCAGAAGGCCCGGCAGACCCUGGAGUU |
| | CUACCCCUGCACCAGCGAGGAGAUCGACCACGAAGAUAUCACCAAAGAUAAGACCAGCA |
| | CCGUGGAGGCCUGCCUGCCCCUGGAGCUGACCAAGAACGAGAGCUGCCUGAACAGCAGA |
| | GAGACCAGCUUCAUCACCAACGGCAGCUGCCUGGCCAGCAGAAAGACCAGCUUCAUGAU |
| | GGCCCUGUGCCUGAGCAGCAUCUACGAGGACCUGAAGAUGUACCAGGUGGAGUUCAAGA |
| | CCAUGAACGCCAAGCUGCUGAUGGACCCCAAGCGGCAGAUCUUCCUGGACCAGAACAUG |
| | CUGGCCGUGAUCGACGAGCUGAUGCAGGCCCUGAACUUCAACAGCGAGACCGUGCCCCA |
| | GAAGAGCAGCCUGGAGGAGCCCGACUUCUACAAGACCAAGAUCAAGCUGUGCAUCCUGC |
| | UGCACGCCUUCAGAAUCAGAGCCGUGACCAUCGACAGAGUGAUGAGCUACCUGAACGCC |
| | AGC |
| | hIL12AB_041 mRNA ORF |
| 47 | TYCFAPRCRERRRNERLRRESVRPV |
| | Human CD80 Intracellular domain |
| 48 | QKKPRYEIRWKVIESVSSDGHEYIYVDPMQLPYDSTWELPRDQLVLGRTLGSGAFGQVV |
| | EATAHGLSHSQATMKVAVKMLKSTARSSEKQALMSELKIMSHLGPHLNVVNLLGACTKG |
| | GPIYIITEYCRYGDLVDYLHRNKHTFLQHHSDKRRPPSAELYSNALPVGLPLPSHVSLT |
| | GESDGGYMDMSKDESVDYVPMLDMKGDVKYADIESSNYMAPYDNYVPSAPERTCRATLI |
| | NESPVLSYMDLVGFSYQVANGMEFLASKNCVHRDLAARNVLICEGKLVKICDFGLARDI |
| | MRDSNYISKGSTFLPLKWMAPESIFNSLYTTLSDVWSFGILLWEIFTLGGTPYPELPMN |
| | EQFYNAIKRGYRMAQPAHASDEIYEIMQKCWEEKFEIRPPFSQLVLLLERLLGEGYKKK |
| | YQQVDEEFLRSDHPAILRSQARLPGFHGLRSPLDTSSVLYTAVQPNEGDNDYIIPLPDP |
| | KPEVADEGPLEGSPSLASSTLNEVNTSSTISCDSPLEPQDEPEPEPQLELQVEPEPELE |
| | QLPDSGCPAPRAEAEDSFL |
| | Human PGFRB Intracellular domain (WT) |
| 49 | QKKPRYEIRWKVIESVSSDGHE |
| | Human PGFRB Intracellular domain (E570tr) |
| 50 | QKKPRYEIRWKVIESVSSDGHEFIFVDPMQLPYDSTWELPRDQLVLGRTLGSGAFGQVV |
| | EATAHGLSHSQATMKVAVAMLKSTARSSEKQALMSELKIMSHLGPHLNVVNLLGACTKG |
| | GPIYIITEYCRYGDLVDYLHRNKHTFLQHHSDKRRPPSAELYSNALPVGLPLPSHVSLT |
| | GESDGG |
| | Human PGFRB Intracellular domain (G739tr) |
| 51 | SGGGSGGGGSGGGGSGGGGSGGGSLQ |
| | Linker |
| 52 | AUGUGCCACCAGCAGCUGGUGAUCAGCUGGUUCAGCCUGGUGUUCCUGGCCAGCCCCCU |
| | GGUGGCCAUCUGGGAGCUGAAGAAGGACGUGUACGUGGUGGAGUUGGAUUGGUACCCCG |
| | ACGCCCCCGGCGAGAUGGUGGUGCUGACCUGCGACACCCCCGAGGAGGACGGCAUCACC |
| | UGGACCCUGGACCAGAGCAGCGAGGUGCUGGGCAGCGGCAAGACCCUGACCAUCCAGGU |
| | GAAGGAGUUCGGCGACGCCGGCCAGUACACCUGCCACAAGGGCGGCGAGGUGCUGAGCC |
| | ACAGCCUGCUGCUGCUGCACAAGAAGGAGGACGGCAUCUGGAGCACCGACAUCCUGAAG |
| | GACCAGAAGGAGCCCAAGAACAAGACCUUCCUGAGAUGCGAGGCCAAGAACUACAGCGG |
| | CAGAUUCACCUGCUGGUGGCUGACCACCAUCAGCACCGACCUGACCUUCAGCGUGAAGA |
| | GCAGCAGAGGCAGCAGCGACCCCCAGGGCGUGACCUGCGGCGCCGCCACCCUGAGCGCC |
| | GAGAGAGUGAGAGGCGACAACAAGGAGUACGAGUACAGCGUGGAGUGCCAGGAAGAUAG |
| | CGCCUGCCCCGCCGCCGAGGAGAGCCUGCCCAUCGAGGUGAUGGUGGACGCCGUGCACA |
| | AGCUGAAGUACGAGAACUACACCAGCAGCUUCUUCAUCAGAGAUAUCAUCAAGCCCGAC |
| | CCCCCCAAGAACCUGCAGCUGAAGCCCCUGAAGAACAGCCGGCAGGUGGAGGUGAGCUG |
| | GGAGUACCCCGACACCUGGAGCACCCCCCACAGCUACUUCAGCCUGACCUUCUGCGUGC |
| | AGGUGCAGGGCAAGAGCAAGAGAGAGAAGAAAGAUAGAGUGUUCACCGACAAGACCAGC |
| | GCCACCGUGAUCUGCAGAAAGAACGCCAGCAUCAGCGUGAGAGCCCAAGAUAGAUACUA |
| | CAGCAGCAGCUGGAGCGAGUGGGCCAGCGUGCCCUGCAGCGGCGGCGGCGGCGGCGGCA |
| | GCAGAAACCUGCCCGUGGCCACCCCCGACCCCGGCAUGUUCCCCUGCCUGCACCACAGC |
| | CAGAACCUGCUGAGAGCCGUGAGCAACAUGCUGCAGAAGGCCCGGCAGACCCUGGAGUU |
| | CUACCCCUGCACCAGCGAGGAGAUCGACCACGAAGAUAUCACCAAAGAUAAGACCAGCA |
| | CCGUGGAGGCCUGCCUGCCCCUGGAGCUGACCAAGAACGAGAGCUGCCUGAACAGCAGA |
| | GAGACCAGCUUCAUCACCAACGGCAGCUGCCUGGCCAGCAGAAAGACCAGCUUCAUGAU |
| | GGCCCUGUGCCUGAGCAGCAUCUACGAGGACCUGAAGAUGUACCAGGUGGAGUUCAAGA |
| | CCAUGAACGCCAAGCUGCUGAUGGACCCCAAGCGGCAGAUCUUCCUGGACCAGAACAUG |
| | CUGGCCGUGAUCGACGAGCUGAUGCAGGCCCUGAACUUCAACAGCGAGACCGUGCCCCA |
| | GAAGAGCAGCCUGGAGGAGCCCGACUUCUACAAGACCAAGAUCAAGCUGUGCAUCCUGC |
| | UGCACGCCUUCAGAAUCAGAGCCGUGACCAUCGACAGAGUGAUGAGCUACCUGAACGCC |
| | AGCUCUGGUGGCGGAUCAGGCGGCGGCGGUUCAGGAGGCGGUGGAAGUGGAGGUGGCGG |
| | GUCUGGCGGAGGUUCACUGCAGAUCUACAUCUGGGCUCCACUGGCCGGCACCUGCGGCG |
| | UGCUGCUGCUGAGCCUGGUGAUCACCCUGUACUGCUACGGGAAACCAAUUCCAAAUCCC |
| | CUCCUGGGGUUGGAUAGCACC |
| | hIL12AB-8TM Nucleotide Sequence |
| 53 | MCHQQLVISWFSLVFLASPLVAIWELKKDVYVVELDWYPDAPGEMVVLTCDTPEEDGIT |
| | WTLDQSSEVLGSGKTLTIQVKEFGDAGQYTCHKGGEVLSHSLLLLHKKEDGIWSTDILK |
| | DQKEPKNKTFLRCEAKNYSGRFTCWWLTTISTDLTFSVKSSRGSSDPQGVTCGAATLSA |
| | ERVRGDNKEYEYSVECQEDSACPAAEESLPIEVMVDAVHKLKYENYTSSFFIRDIIKPD |
| | PPKNLQLKPLKNSRQVEVSWEYPDTWSTPHSYFSLTFCVQVQGKSKREKKDRVFTDKTS |
| | 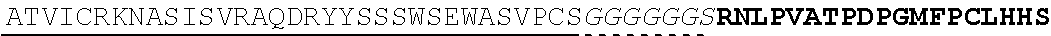 |
| | QNLLRAVSNMLQKARQTLEFYPCTSEEIDHEDITKDKTSTVEACLPLELTKNESCLNSR |
| | ETSFITNGSCLASRKTSFMMALCLSSIYEDLKMYQVEFKTMNAKLLMDPKRQIFLDQNM |
| | LAVIDELMQALNFNSETVPQKSSLEEPDFYKTKIKLCILLHAFRIRAVTIDRVMSYLNA |
| | 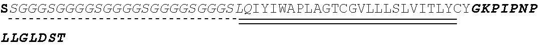 |
| | Italic: signal peptide; Underline: IL12B; Dashed underline |
| | and Italic: linker; Bold: IL12A; Double underline: |
| | Transmembrane domain; Bold and Italic: Epitope tag |
| | hIL12AB-8TM Amino Acid Sequence |
| 54 | AUGUGCCACCAGCAGCUGGUGAUCAGCUGGUUCAGCCUGGUGUUCCUGGCCAGCCCCCU |
| | GGUGGCCAUCUGGGAGCUGAAGAAGGACGUGUACGUGGUGGAGUUGGAUUGGUACCCCG |
| | ACGCCCCCGGCGAGAUGGUGGUGCUGACCUGCGACACCCCCGAGGAGGACGGCAUCACC |
| | UGGACCCUGGACCAGAGCAGCGAGGUGCUGGGCAGCGGCAAGACCCUGACCAUCCAGGU |
| | GAAGGAGUUCGGCGACGCCGGCCAGUACACCUGCCACAAGGGCGGCGAGGUGCUGAGCC |
| | ACAGCCUGCUGCUGCUGCACAAGAAGGAGGACGGCAUCUGGAGCACCGACAUCCUGAAG |
| | GACCAGAAGGAGCCCAAGAACAAGACCUUCCUGAGAUGCGAGGCCAAGAACUACAGCGG |
| | CAGAUUCACCUGCUGGUGGCUGACCACCAUCAGCACCGACCUGACCUUCAGCGUGAAGA |
| | GCAGCAGAGGCAGCAGCGACCCCCAGGGCGUGACCUGCGGCGCCGCCACCCUGAGCGCC |
| | GAGAGAGUGAGAGGCGACAACAAGGAGUACGAGUACAGCGUGGAGUGCCAGGAAGAUAG |
| | CGCCUGCCCCGCCGCCGAGGAGAGCCUGCCCAUCGAGGUGAUGGUGGACGCCGUGCACA |
| | AGCUGAAGUACGAGAACUACACCAGCAGCUUCUUCAUCAGAGAUAUCAUCAAGCCCGAC |
| | CCCCCCAAGAACCUGCAGCUGAAGCCCCUGAAGAACAGCCGGCAGGUGGAGGUGAGCUG |
| | GGAGUACCCCGACACCUGGAGCACCCCCCACAGCUACUUCAGCCUGACCUUCUGCGUGC |
| | AGGUGCAGGGCAAGAGCAAGAGAGAGAAGAAAGAUAGAGUGUUCACCGACAAGACCAGC |
| | GCCACCGUGAUCUGCAGAAAGAACGCCAGCAUCAGCGUGAGAGCCCAAGAUAGAUACUA |
| | CAGCAGCAGCUGGAGCGAGUGGGCCAGCGUGCCCUGCAGCGGCGGCGGCGGCGGCGGCA |
| | GCAGAAACCUGCCCGUGGCCACCCCCGACCCCGGCAUGUUCCCCUGCCUGCACCACAGC |
| | CAGAACCUGCUGAGAGCCGUGAGCAACAUGCUGCAGAAGGCCCGGCAGACCCUGGAGUU |
| | CUACCCCUGCACCAGCGAGGAGAUCGACCACGAAGAUAUCACCAAAGAUAAGACCAGCA |
| | CCGUGGAGGCCUGCCUGCCCCUGGAGCUGACCAAGAACGAGAGCUGCCUGAACAGCAGA |
| | GAGACCAGCUUCAUCACCAACGGCAGCUGCCUGGCCAGCAGAAAGACCAGCUUCAUGAU |
| | GGCCCUGUGCCUGAGCAGCAUCUACGAGGACCUGAAGAUGUACCAGGUGGAGUUCAAGA |
| | CCAUGAACGCCAAGCUGCUGAUGGACCCCAAGCGGCAGAUCUUCCUGGACCAGAACAUG |
| | CUGGCCGUGAUCGACGAGCUGAUGCAGGCCCUGAACUUCAACAGCGAGACCGUGCCCCA |
| | GAAGAGCAGCCUGGAGGAGCCCGACUUCUACAAGACCAAGAUCAAGCUGUGCAUCCUGC |
| | UGCACGCCUUCAGAAUCAGAGCCGUGACCAUCGACAGAGUGAUGAGCUACCUGAACGCC |
| | AGCUCUGGUGGCGGAUCAGGCGGCGGCGGUUCAGGAGGCGGUGGAAGUGGAGGUGGCGG |
| | GUCUGGCGGAGGUUCACUGCAGAUCUACAUCUGGGCUCCACUGGCCGGCACCUGCGGCG |
| | UGCUGCUGCUGAGCCUGGUGAUCACCCUGUACUGCUAC |
| | hIL12AB-8TM no epitope tag Nucleotide Sequence |
| 55 | MCHQQLVISWFSLVFLASPLVAIWELKKDVYVVELDWYPDAPGEMVVLTCDTPEEDGIT |
| | WTLDQSSEVLGSGKTLTIQVKEFGDAGQYTCHKGGEVLSHSLLLLHKKEDGIWSTDILK |
| | DQKEPKNKTFLRCEAKNYSGRFTCWWLTTISTDLTFSVKSSRGSSDPQGVTCGAATLSA |
| | ERVRGDNKEYEYSVECQEDSACPAAEESLPIEVMVDAVHKLKYENYTSSFFIRDIIKPD |
| | PPKNLQLKPLKNSRQVEVSWEYPDTWSTPHSYFSLTFCVQVQGKSKREKKDRVFTDKTS |
| | 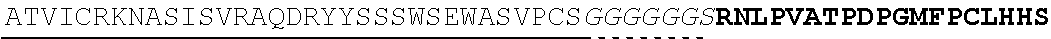 |
| | QNLLRAVSNMLQKARQTLEFYPCTSEEIDHEDITKDKTSTVEACLPLELTKNESCLNSR |
| | ETSFITNGSCLASRKTSFMMALCLSSIYEDLKMYQVEFKTMNAKLLMDPKRQIFLDQNM |
| | LAVIDELMQALNFNSETVPQKSSLEEPDFYKTKIKLCILLHAFRIRAVTIDRVMSYLNA |
| | 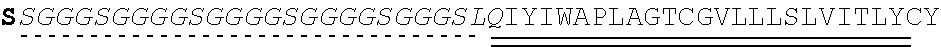 |
| | Italic: signal peptide; Underline: IL12B; Dashed underline |
| | and Italic: linker; Bold: IL12A; Double underline: |
| | Transmembrane domain |
| | hIL12AB-8TM no epitope tag Amino Acid Sequence |
| 56 | AUGUGCCACCAGCAGCUGGUGAUCAGCUGGUUCAGCCUGGUGUUCCUGGCCAGCCCCCU |
| | GGUGGCCAUCUGGGAGCUGAAGAAGGACGUGUACGUGGUGGAGUUGGAUUGGUACCCCG |
| | ACGCCCCCGGCGAGAUGGUGGUGCUGACCUGCGACACCCCCGAGGAGGACGGCAUCACC |
| | UGGACCCUGGACCAGAGCAGCGAGGUGCUGGGCAGCGGCAAGACCCUGACCAUCCAGGU |
| | GAAGGAGUUCGGCGACGCCGGCCAGUACACCUGCCACAAGGGCGGCGAGGUGCUGAGCC |
| | ACAGCCUGCUGCUGCUGCACAAGAAGGAGGACGGCAUCUGGAGCACCGACAUCCUGAAG |
| | GACCAGAAGGAGCCCAAGAACAAGACCUUCCUGAGAUGCGAGGCCAAGAACUACAGCGG |
| | CAGAUUCACCUGCUGGUGGCUGACCACCAUCAGCACCGACCUGACCUUCAGCGUGAAGA |
| | GCAGCAGAGGCAGCAGCGACCCCCAGGGCGUGACCUGCGGCGCCGCCACCCUGAGCGCC |
| | GAGAGAGUGAGAGGCGACAACAAGGAGUACGAGUACAGCGUGGAGUGCCAGGAAGAUAG |
| | CGCCUGCCCCGCCGCCGAGGAGAGCCUGCCCAUCGAGGUGAUGGUGGACGCCGUGCACA |
| | AGCUGAAGUACGAGAACUACACCAGCAGCUUCUUCAUCAGAGAUAUCAUCAAGCCCGAC |
| | CCCCCCAAGAACCUGCAGCUGAAGCCCCUGAAGAACAGCCGGCAGGUGGAGGUGAGCUG |
| | GGAGUACCCCGACACCUGGAGCACCCCCCACAGCUACUUCAGCCUGACCUUCUGCGUGC |
| | AGGUGCAGGGCAAGAGCAAGAGAGAGAAGAAAGAUAGAGUGUUCACCGACAAGACCAGC |
| | GCCACCGUGAUCUGCAGAAAGAACGCCAGCAUCAGCGUGAGAGCCCAAGAUAGAUACUA |
| | CAGCAGCAGCUGGAGCGAGUGGGCCAGCGUGCCCUGCAGCGGCGGCGGCGGCGGCGGCA |
| | GCAGAAACCUGCCCGUGGCCACCCCCGACCCCGGCAUGUUCCCCUGCCUGCACCACAGC |
| | CAGAACCUGCUGAGAGCCGUGAGCAACAUGCUGCAGAAGGCCCGGCAGACCCUGGAGUU |
| | CUACCCCUGCACCAGCGAGGAGAUCGACCACGAAGAUAUCACCAAAGAUAAGACCAGCA |
| | CCGUGGAGGCCUGCCUGCCCCUGGAGCUGACCAAGAACGAGAGCUGCCUGAACAGCAGA |
| | GAGACCAGCUUCAUCACCAACGGCAGCUGCCUGGCCAGCAGAAAGACCAGCUUCAUGAU |
| | GGCCCUGUGCCUGAGCAGCAUCUACGAGGACCUGAAGAUGUACCAGGUGGAGUUCAAGA |
| | CCAUGAACGCCAAGCUGCUGAUGGACCCCAAGCGGCAGAUCUUCCUGGACCAGAACAUG |
| | CUGGCCGUGAUCGACGAGCUGAUGCAGGCCCUGAACUUCAACAGCGAGACCGUGCCCCA |
| | GAAGAGCAGCCUGGAGGAGCCCGACUUCUACAAGACCAAGAUCAAGCUGUGCAUCCUGC |
| | UGCACGCCUUCAGAAUCAGAGCCGUGACCAUCGACAGAGUGAUGAGCUACCUGAACGCC |
| | AGCUCUGGUGGCGGAUCAGGCGGCGGCGGUUCAGGAGGCGGUGGAAGUGGAGGUGGCGG |
| | GUCUGGCGGAGGUUCACUGCAGCUGCUGCCCAGCUGGGCCAUCACCCUGAUCAGCGUGA |
| | ACGGCAUCUUCGUGAUCUGCUGCCUGACCUACUGCUUCGCCCCUCGAUGCAGAGAGAGA |
| | AGAAGAAACGAGAGACUGAGAAGAGAGAGCGUGCGACCCGUG |
| | h12AB-80TID Nucleotide Sequence 1 matched |
| 57 | AUGUGCCACCAGCAGCUGGUGAUCAGCUGGUUCAGCCUGGUGUUCCUGGCCAGCCCUCU |
| | GGUGGCCAUCUGGGAGCUGAAGAAGGACGUGUACGUGGUGGAGCUGGACUGGUACCCUG |
| | ACGCCCCUGGCGAGAUGGUGGUGCUGACCUGCGACACCCCUGAGGAGGACGGCAUCACC |
| | UGGACCCUGGACCAGAGCAGCGAGGUGCUGGGCAGCGGCAAGACCCUGACCAUCCAGGU |
| | GAAGGAGUUCGGCGACGCCGGCCAGUACACCUGCCACAAGGGCGGAGAGGUGUUAAGCC |
| | ACAGCCUGCUCUUGCUACACAAGAAGGAGGACGGUAUUUGGAGCACCGACAUCCUGAAG |
| | GACCAGAAGGAGCCUAAGAACAAGACCUUCCUGAGGUGCGAGGCCAAGAACUACAGCGG |
| | CAGAUUCACCUGCUGGUGGCUGACCACCAUCUCCACGGACCUGACCUUCAGCGUUAAGA |
| | GUAGCAGAGGCAGCAGCGACCCUCAGGGCGUGACUUGUGGCGCCGCCACCCUGAGCGCC |
| | GAGAGAGUGAGAGGCGACAACAAGGAGUACGAGUACAGCGUGGAGUGCCAGGAGGACAG |
| | CGCCUGCCCUGCCGCCGAGGAGAGCCUGCCUAUCGAGGUGAUGGUGGACGCCGUGCACA |
| | AGCUGAAGUACGAGAAUUACACCUCAUCCUUCUUCAUCAGAGACAUCAUCAAGCCUGAC |
| | CCUCCAAAGAAUCUGCAGCUGAAGCCUCUGAAGAACAGCAGACAGGUGGAGGUGAGCUG |
| | GGAGUAUCCGGAUACCUGGAGCACACCUCACAGCUACUUCUCACUUACAUUCUGCGUGC |
| | AGGUGCAGGGCAAGAGCAAGAGAGAGAAGAAGGAUAGAGUGUUCACCGACAAGACCAGC |
| | GCCACCGUGAUCUGCAGAAAGAACGCCAGCAUCUCAGUGAGAGCCCAGGACAGAUACUA |
| | CUCAUCCUCCUGGAGCGAGUGGGCCAGCGUGCCUUGCUCCGGUGGUGGUGGCGGAGGCA |
| | GCAGAAACCUGCCUGUGGCUACACCUGAUCCUGGCAUGUUCCCUUGCCUGCACCACAGC |
| | CAGAACCUGCUGAGAGCCGUGAGCAACAUGCUGCAGAAGGCCAGACAGACUCUGGAGUU |
| | CUACCCUUGCACCAGCGAGGAGAUCGACCACGAGGACAUCACCAAGGAUAAGACAAGCA |
| | CCGUGGAGGCCUGCCUGCCUCUGGAGCUGACCAAGAACGAGAGCUGCCUAAACUCUAGG |
| | GAAACCAGCUUCAUUACUAACGGCAGUUGCUUAGCCAGCCGGAAGACAUCGUUCAUGAU |
| | GGCCCUGUGCCUGAGCAGCAUCUACGAGGACCUGAAGAUGUACCAAGUGGAGUUCAAGA |
| | CCAUGAACGCCAAGCUGCUGAUGGACCCUAAGAGACAGAUCUUCCUAGACCAGAACAUG |
| | CUCGCCGUGAUCGACGAGCUGAUGCAGGCCCUGAACUUCAACAGCGAAACUGUGCCUCA |
| | GAAGAGUUCACUGGAGGAGCCUGACUUCUAUAAGACUAAGAUCAAGCUGUGUAUUCUCC |
| | UCCACGCCUUCAGAAUCAGGGCUGUCACCAUCGAUAGGGUGAUGAGCUACCUGAACGCA |
| | UCGUCCGGCGGAGGAUCCGGAGGAGGAGGCUCCGGCGGUGGUGGAAGUGGAGGAGGUGG |
| | AUCAGGAGGCGGUAGUCUCCAGCUCCUGCCUAGCUGGGCCAUCACCCUGAUCUCUGUAA |
| | ACGGCAUUUUCGUCAUUUGCUGUCUGACUUACUGCUUCGCCCCUAGGUGCCGGGAGCGU |
| | AGGAGAAACGAGAGACUGCGCCGGGAGUCCGUGCGGCCUGUG |
| | h12AB-80TID Nucleotide Sequence 2 IL12_041 |
| 58 | AUGUGUCACCAGCAGCUCGUGAUCAGCUGGUUCAGCCUGGUGUUCCUGGCCUCCCCGCU |
| | GGUGGCCAUCUGGGAGCUGAAGAAGGACGUGUACGUGGUGGAGCUGGACUGGUACCCCG |
| | ACGCUCCCGGCGAGAUGGUGGUGCUGACCUGCGACACACCGGAGGAAGACGGAAUCACC |
| | UGGACCCUGGACCAAUCCUCCGAAGUUCUGGGCAGCGGCAAGACCCUGACCAUCCAGGU |
| | GAAGGAGUUCGGCGACGCCGGCCAGUACACCUGCCACAAGGGCGGCGAGGUGCUGAGCC |
| | ACAGCCUGCUCCUCCUCCACAAGAAAGAGGACGGCAUCUGGAGCACCGACAUCCUGAAG |
| | GACCAGAAGGAGCCCAAGAACAAGACCUUCCUGCGGUGCGAGGCCAAGAACUACUCAGG |
| | CCGAUUCACCUGUUGGUGGCUCACAACUAUCAGCACAGACCUGACCUUCAGCGUGAAGU |
| | CUAGCCGGGGCAGCAGCGAUCCUCAGGGCGUGACCUGCGGCGCCGCCACCCUGAGCGCC |
| | GAGCGGGUGCGGGGCGACAACAAGGAGUACGAGUACAGCGUGGAGUGCCAGGAGGACAG |
| | CGCCUGCCCGGCCGCCGAAGAGUCCCUGCCCAUCGAGGUGAUGGUGGACGCCGUGCACA |
| | AACUCAAGUACGAGAACUACACCUCCAGCUUCUUCAUCCGGGACAUCAUCAAGCCCGAU |
| | CCGCCCAAGAACCUGCAGCUGAAGCCCCUGAAGAACAGCCGGCAGGUGGAGGUGAGCUG |
| | GGAAUACCCGGACACGUGGUCCACCCCACACAGCUACUUCAGCCUGACCUUUUGCGUGC |
| | AGGUCCAAGGCAAGAGCAAGCGGGAGAAGAAGGACCGGGUGUUCACCGAUAAGACCUCA |
| | GCCACCGUGAUUUGCAGAAAGAACGCAUCCAUAUCCGUACGCGCCCAGGAUCGGUACUA |
| | CAGCAGCAGCUGGAGCGAGUGGGCCAGCGUGCCCUGUAGCGGCGGCGGCGGUGGUGGGA |
| | GUCGCAACCUGCCCGUGGCCACCCCGGACCCCGGCAUGUUCCCCUGCCUGCACCACAGC |
| | CAGAACCUGCUGCGGGCCGUGAGCAACAUGCUGCAGAAGGCCCGGCAGACCCUGGAGUU |
| | CUACCCCUGCACCAGCGAGGAGAUCGACCACGAGGACAUCACCAAGGACAAGACCAGCA |
| | CCGUGGAGGCCUGCCUGCCCCUGGAGCUGACCAAGAACGAGAGCUGCCUGAACAGUCGC |
| | GAAACCUCCUUCAUUACGAACGGCAGCUGCCUGGCCAGCCGGAAGACCAGCUUCAUGAU |
| | GGCCCUGUGCCUGAGCAGCAUCUACGAGGACCUGAAGAUGUACCAGGUGGAGUUCAAGA |
| | CCAUGAACGCCAAGCUGCUGAUGGACCCCAAGCGGCAGAUCUUCCUGGACCAGAACAUG |
| | CUGGCCGUGAUCGACGAGCUGAUGCAGGCCCUGAACUUCAACAGCGAAACCGUGCCCCA |
| | GAAGUCCAGCCUGGAGGAGCCCGACUUCUACAAGACCAAGAUCAAGCUGUGCAUCCUGC |
| | UGCACGCCUUCCGGAUCCGGGCCGUGACCAUCGACCGGGUGAUGAGCUACCUGAACGCC |
| | UCUUCCGGUGGCGGGAGCGGAGGCGGUGGAUCUGGCGGAGGAGGGUCGGGAGGCGGCGG |
| | AAGCGGUGGUGGAAGCCUUCAACUGCUGCCCUCGUGGGCCAUCACACUGAUCUCCGUGA |
| | ACGGCAUCUUCGUGAUCUGCUGCCUGACCUACUGCUUCGCCCCUCGGUGCCGCGAGCGA |
| | CGGAGAAACGAGAGGCUCAGACGGGAGAGCGUGCGGCCCGUG |
| | h12AB-80TID Nucleotide Sequence 3 IL12_042 |
| 59 | AUGUGCCACCAACAGCUGGUGAUCAGCUGGUUCAGCCUGGUGUUCCUGGCCUCACCCCU |
| | GGUGGCCAUCUGGGAGCUGAAGAAGGACGUGUACGUGGUGGAGCUGGACUGGUACCCCG |
| | ACGCCCCUGGCGAGAUGGUGGUGCUGACCUGCGACACGCCCGAGGAAGACGGUAUCACC |
| | UGGACUCUGGACCAGAGCAGCGAGGUGCUGGGCAGCGGCAAGACCCUGACCAUCCAGGU |
| | GAAGGAGUUCGGCGACGCCGGCCAGUACACCUGCCACAAGGGCGGCGAAGUGCUGAGCC |
| | ACAGCCUUCUGCUGCUGCACAAGAAGGAGGACGGCAUUUGGAGCACCGACAUCCUGAAG |
| | GACCAGAAGGAGCCCAAGAACAAGACCUUCCUGCGGUGCGAGGCCAAGAACUACAGCGG |
| | CCGGUUCACCUGCUGGUGGCUGACCACCAUCAGCACCGACCUUACCUUCAGCGUUAAGA |
| | GCAGCCGGGGCAGCAGCGAUCCCCAGGGCGUGACCUGCGGAGCCGCCACCCUCUCCGCA |
| | GAGCGGGUGCGUGGCGACAACAAGGAGUACGAGUACAGCGUGGAGUGCCAGGAGGAUAG |
| | CGCCUGUCCCGCUGCCGAAGAGAGCCUGCCCAUCGAGGUGAUGGUGGACGCCGUGCACA |
| | AGCUGAAGUACGAGAACUACACCAGCAGCUUCUUCAUCCGGGACAUCAUCAAGCCCGAU |
| | CCACCCAAGAACCUGCAGCUGAAGCCCCUGAAGAACAGCAGGCAGGUUGAGGUGAGCUG |
| | GGAAUACCCCGACACCUGGAGCACCCCUCACAGCUACUUCAGCCUGACCUUCUGCGUGC |
| | AGGUGCAGGGCAAGAGCAAGCGGGAGAAGAAGGAUCGGGUGUUCACCGAUAAGACCAGC |
| | GCCACCGUGAUCUGCCGGAAGAACGCCAGCAUCAGCGUUCGGGCCCAGGACCGGUACUA |
| | CAGCAGCAGCUGGAGCGAGUGGGCCAGCGUGCCCUGCUCUGGAGGCGGAGGCGGAGGCU |
| | CACGGAACCUGCCAGUGGCCACGCCGGAUCCCGGCAUGUUCCCCUGCCUGCACCACAGC |
| | CAGAACCUGCUGCGGGCCGUGAGCAACAUGCUGCAGAAGGCCCGGCAGACCCUGGAGUU |
| | CUACCCCUGCACCAGCGAGGAGAUCGACCACGAGGACAUCACCAAGGACAAGACCAGCA |
| | CCGUGGAGGCCUGCCUGCCCCUGGAGCUGACCAAGAACGAGAGCUGCCUGAACAGCCGG |
| | GAGACAAGCUUCAUCACCAACGGCAGCUGCCUGGCCAGCCGGAAGACCAGCUUCAUGAU |
| | GGCCCUGUGCCUGAGCAGCAUCUACGAGGACCUGAAGAUGUACCAGGUGGAGUUCAAGA |
| | CCAUGAACGCCAAGCUGCUGAUGGACCCCAAGCGGCAGAUCUUCCUGGAUCAGAACAUG |
| | CUGGCCGUGAUCGACGAGCUGAUGCAGGCCCUGAACUUCAACAGCGAGACUGUGCCCCA |
| | GAAGUCCAGCCUGGAGGAGCCCGACUUCUACAAGACCAAGAUCAAGCUGUGCAUCCUGC |
| | UGCACGCCUUCCGGAUCCGGGCUGUGACCAUCGACCGGGUGAUGAGCUACCUGAACGCC |
| | UCUUCCGGCGGCGGAUCGGGAGGUGGAGGUUCUGGAGGAGGUGGAAGCGGUGGUGGCGG |
| | AAGCGGCGGUGGCAGCCUGCAAUUGCUCCCCAGCUGGGCCAUCACCCUGAUCAGCGUGA |
| | ACGGCAUCUUCGUGAUCUGUUGCCUGACCUACUGCUUCGCCCCACGGUGCCGGGAGAGA |
| | CGGCGGAACGAGCGGCUGCGGCGAGAGAGCGUGCGGCCCGUG |
| | h12AB-80TID Nucleotide Sequence 4 IL12_043 |
| 60 | AUGUGUCACCAGCAGUUGGUGAUAUCUUGGUUCUCACUGGUGUUUCUUGCAUCACCACU |
| | CGUGGCGAUCUGGGAACUUAAGAAGGACGUCUACGUGGUGGAGUUAGAUUGGUAUCCUG |
| | ACGCACCCGGGGAAAUGGUUGUCCUCACGUGCGACACUCCAGAGGAAGACGGGAUCACC |
| | UGGACCCUGGAUCAGUCGUCAGAGGUACUUGGCAGUGGCAAGACACUGACAAUCCAGGU |
| | UAAAGAGUUUGGUGACGCCGGGCAGUAUACGUGCCACAAGGGCGGCGAGGUGUUGUCAC |
| | AUUCUCUGCUUCUCCUGCACAAGAAAGAAGACGGCAUCUGGUCAACUGACAUCCUGAAA |
| | GACCAGAAAGAACCCAAGAAUAAGACCUUCCUCCGUUGCGAAGCAAAGAACUACUCAGG |
| | GCGUUUCACUUGCUGGUGGCUAACCACAAUUUCUACCGAUCUGACGUUCUCUGUGAAGU |
| | CAAGUAGGGGAUCCUCAGACCCUCAAGGGGUCACCUGCGGCGCCGCCACCUUAUCCGCC |
| | GAAAGAGUUCGGGGUGACAAUAAAGAGUACGAGUACAGCGUCGAGUGUCAGGAGGACUC |
| | CGCCUGUCCUGCUGCAGAGGAGUCCCUGCCGAUCGAAGUUAUGGUGGACGCCGUCCACA |
| | AGCUCAAAUACGAGAACUACACCUCAAGCUUCUUCAUCAGAGACAUCAUCAAGCCUGAU |
| | CCACCCAAGAACCUGCAACUGAAGCCUUUGAAGAACAGCCGACAGGUGGAGGUUUCUUG |
| | GGAAUAUCCAGACACGUGGAGUACGCCCCAUUCCUACUUCAGCUUGACCUUCUGCGUGC |
| | AGGUUCAGGGGAAGUCCAAGAGAGAGAAGAAGGAUCGUGUGUUCACAGACAAGACCUCC |
| | GCCACCGUGAUCUGCCGGAAGAACGCAUCUAUCAGUGUUAGGGCCCAGGAUCGGUACUA |
| | CUCGAGUUCCUGGUCUGAGUGGGCAAGUGUGCCCUGCUCCGGUGGCGGCGGAGGAGGGU |
| | CAAGGAACCUGCCCGUUGCCACACCAGAUCCAGGAAUGUUCCCCUGUCUGCACCACUCU |
| | CAGAACCUUUUGCGAGCCGUUUCUAAUAUGCUUCAGAAGGCUCGGCAGACCCUUGAGUU |
| | UUAUCCCUGCACGUCUGAGGAGAUCGAUCACGAGGACAUCACCAAGGACAAGACUUCCA |
| | CCGUUGAAGCCUGUUUACCUCUGGAACUGACCAAGAACGAAUCCUGUCUCAACAGUAGG |
| | GAAACGAGCUUCAUCACUAACGGAAGCUGUCUGGCUAGCCGGAAGACCUCUUUUAUGAU |
| | GGCCCUGUGCUUGAGCUCUAUUUACGAAGAUUUGAAGAUGUACCAAGUGGAAUUUAAGA |
| | CUAUGAACGCCAAACUGCUGAUGGACCCUAAGCGCCAAAUCUUCUUGGAUCAGAAUAUG |
| | CUGGCUGUAAUCGACGAGCUCAUGCAGGCUCUGAACUUCAACAGCGAGACGGUACCGCA |
| | GAAGAGUUCCCUGGAAGAACCGGACUUCUACAAGACUAAGAUUAAACUCUGCAUACUCC |
| | UCCACGCCUUCCGGAUCAGGGCCGUCACAAUAGAUAGGGUCAUGAGUUAUCUUAACGCG |
| | AGUUCUGGUGGUGGAUCGGGUGGCGGAGGCUCAGGAGGAGGCGGUUCUGGCGGUGGUGG |
| | GAGUGGAGGCGGUAGUCUGCAGCUGCUGCCGAGUUGGGCAAUCACGCUAAUCAGCGUGA |
| | ACGGAAUAUUCGUAAUUUGUUGCCUCACCUAUUGUUUCGCACCCAGGUGCAGGGAAAGG |
| | AGGCGAAACGAAAGGUUGAGGAGGGAAUCUGUCCGGCCAGUG |
| | h12AB-80TID Nucleotide Sequence 5 IL12_044 |
| 61 | MCHQQLVISWFSLVFLASPLVAIWELKKDVYVVELDWYPDAPGEMVVLTCDTPEEDGIT |
| | WTLDQSSEVLGSGKTLTIQVKEFGDAGQYTCHKGGEVLSHSLLLLHKKEDGIWSTDILK |
| | DQKEPKNKTFLRCEAKNYSGRFTCWWLTTISTDLTFSVKSSRGSSDPQGVTCGAATLSA |
| | ERVRGDNKEYEYSVECQEDSACPAAEESLPIEVMVDAVHKLKYENYTSSFFIRDIIKPD |
| | PPKNLQLKPLKNSRQVEVSWEYPDTWSTPHSYFSLTFCVQVQGKSKREKKDRVFTDKTS |
| | 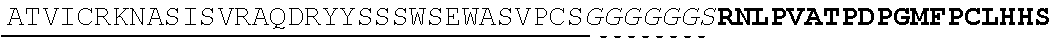 |
| | QNLLRAVSNMLQKARQTLEFYPCTSEEIDHEDITKDKTSTVEACLPLELTKNESCLNSR |
| | ETSFITNGSCLASRKTSFMMALCLSSIYEDLKMYQVEFKTMNAKLLMDPKRQIFLDQNM |
| | LAVIDELMQALNFNSETVPQKSSLEEPDFYKTKIKLCILLHAFRIRAVTIDRVMSYLNA |
| | 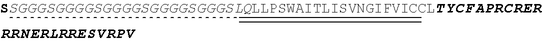 |
| | Italic: signal peptide; Underline: IL12B; Dashed underline |
| | and Italic: linker; Bold: IL12A; Double underline: |
| | Transmembrane domain; Bold and Italic: Intracellular domain |
| | h12AB-80TID Amino Acid Sequence 1 (Corresponds to nucleotide |
| | sequences 1- |
| 62 | GUGGUGGUGAUCAGCGCCAUCCUGGCCCUGGUGGUGCUGACCAUCAUCAGCCUGAUCAU |
| | CCUGAUCAUGCUGUGG |
| | Human PGFRB transmembrane domain nucleotide sequence |
| 63 | CAGAAGAAGCCCAGAUACGAGAUCAGAUGGAAGGUGAUCGAGAGCGUGAGCAGCGACGG |
| | CCACGAG |
| | Human PGFRB E570tr intracellular domain nucleotide sequence |
| 64 | CAGAAGAAGCCCAGAUACGAGAUCCGGUGGAAGGUGAUCGAGAGCGUGAGCAGCGACGG |
| | CCACGAGUUCAUCUUCGUGGACCCCAUGCAGCUGCCCUACGACAGCACCUGGGAGCUGC |
| | CCCGUGAUCAGCUGGUGCUGGGCAGAACCCUGGGCAGCGGCGCCUUCGGCCAGGUGGUG |
| | GAGGCUACCGCCCACGGCCUGAGCCACAGCCAGGCCACCAUGAAGGUGGCCGUGGCCAU |
| | GCUCAAGAGCACCGCCAGAAGCAGCGAGAAGCAGGCCCUGAUGAGCGAGCUGAAGAUCA |
| | UGAGCCAUCUGGGGCCCCACCUGAACGUGGUGAACCUGCUGGGCGCCUGCACCAAGGGC |
| | GGCCCCAUCUACAUCAUCACCGAGUACUGCAGAUACGGCGACCUGGUGGACUACCUGCA |
| | CAGAAACAAGCACACCUUCCUGCAGCACCACAGCGACAAGAGAAGACCUCCCAGCGCCG |
| | AGCUGUACAGCAACGCCCUGCCCGUUGGUCUGCCCCUACCCAGCCACGUGAGCCUGACC |
| | GGCGAGAGCGACGGCGGC |
| | Human PGFRB G739tr intracellular domain nucleotide sequence |
| 65 | AUGUGCCACCAGCAGCUGGUGAUCAGCUGGUUCAGCCUGGUGUUCCUGGCCAGCCCCCU |
| | GGUGGCCAUCUGGGAGCUGAAGAAGGACGUGUACGUGGUGGAGUUGGAUUGGUACCCCG |
| | ACGCCCCCGGCGAGAUGGUGGUGCUGACCUGCGACACCCCCGAGGAGGACGGCAUCACC |
| | UGGACCCUGGACCAGAGCAGCGAGGUGCUGGGCAGCGGCAAGACCCUGACCAUCCAGGU |
| | GAAGGAGUUCGGCGACGCCGGCCAGUACACCUGCCACAAGGGCGGCGAGGUGCUGAGCC |
| | ACAGCCUGCUGCUGCUGCACAAGAAGGAGGACGGCAUCUGGAGCACCGACAUCCUGAAG |
| | GACCAGAAGGAGCCCAAGAACAAGACCUUCCUGAGAUGCGAGGCCAAGAACUACAGCGG |
| | CAGAUUCACCUGCUGGUGGCUGACCACCAUCAGCACCGACCUGACCUUCAGCGUGAAGA |
| | GCAGCAGAGGCAGCAGCGACCCCCAGGGCGUGACCUGCGGCGCCGCCACCCUGAGCGCC |
| | GAGAGAGUGAGAGGCGACAACAAGGAGUACGAGUACAGCGUGGAGUGCCAGGAAGAUAG |
| | CGCCUGCCCCGCCGCCGAGGAGAGCCUGCCCAUCGAGGUGAUGGUGGACGCCGUGCACA |
| | AGCUGAAGUACGAGAACUACACCAGCAGCUUCUUCAUCAGAGAUAUCAUCAAGCCCGAC |
| | CCCCCCAAGAACCUGCAGCUGAAGCCCCUGAAGAACAGCCGGCAGGUGGAGGUGAGCUG |
| | GGAGUACCCCGACACCUGGAGCACCCCCCACAGCUACUUCAGCCUGACCUUCUGCGUGC |
| | AGGUGCAGGGCAAGAGCAAGAGAGAGAAGAAAGAUAGAGUGUUCACCGACAAGACCAGC |
| | GCCACCGUGAUCUGCAGAAAGAACGCCAGCAUCAGCGUGAGAGCCCAAGAUAGAUACUA |
| | CAGCAGCAGCUGGAGCGAGUGGGCCAGCGUGCCCUGCAGCGGCGGCGGCGGCGGCGGCA |
| | GCAGAAACCUGCCCGUGGCCACCCCCGACCCCGGCAUGUUCCCCUGCCUGCACCACAGC |
| | CAGAACCUGCUGAGAGCCGUGAGCAACAUGCUGCAGAAGGCCCGGCAGACCCUGGAGUU |
| | CUACCCCUGCACCAGCGAGGAGAUCGACCACGAAGAUAUCACCAAAGAUAAGACCAGCA |
| | CCGUGGAGGCCUGCCUGCCCCUGGAGCUGACCAAGAACGAGAGCUGCCUGAACAGCAGA |
| | GAGACCAGCUUCAUCACCAACGGCAGCUGCCUGGCCAGCAGAAAGACCAGCUUCAUGAU |
| | GGCCCUGUGCCUGAGCAGCAUCUACGAGGACCUGAAGAUGUACCAGGUGGAGUUCAAGA |
| | CCAUGAACGCCAAGCUGCUGAUGGACCCCAAGCGGCAGAUCUUCCUGGACCAGAACAUG |
| | CUGGCCGUGAUCGACGAGCUGAUGCAGGCCCUGAACUUCAACAGCGAGACCGUGCCCCA |
| | GAAGAGCAGCCUGGAGGAGCCCGACUUCUACAAGACCAAGAUCAAGCUGUGCAUCCUGC |
| | UGCACGCCUUCAGAAUCAGAGCCGUGACCAUCGACAGAGUGAUGAGCUACCUGAACGCC |
| | AGCUCUGGUGGCGGAUCAGGGGGUGGCGGAUCUGGCGGGGGUGGAAGUGGAGGUGGCGG |
| | GUCUGGCGGAGGUUCACUGCAGGUGGUGGUGAUCAGCGCCAUCCUGGCCCUGGUGGUGC |
| | UGACCAUCAUCAGCCUGAUCAUCCUGAUCAUGCUGUGGCAGAAGAAGCCCAGAUACGAG |
| | AUCAGAUGGAAGGUGAUCGAGAGCGUGAGCAGCGACGGCCACGAG |
| | h12AB-PTM-ICD-E570tr Nucleotide Sequence |
| 66 | MCHQQLVISWFSLVFLASPLVAIWELKKDVYVVELDWYPDAPGEMVVLTCDTPEEDGIT |
| | WTLDQSSEVLGSGKTLTIQVKEFGDAGQYTCHKGGEVLSHSLLLLHKKEDGIWSTDILK |
| | DQKEPKNKTFLRCEAKNYSGRFTCWWLTTISTDLTFSVKSSRGSSDPQGVTCGAATLSA |
| | ERVRGDNKEYEYSVECQEDSACPAAEESLPIEVMVDAVHKLKYENYTSSFFIRDIIKPD |
| | PPKNLQLKPLKNSRQVEVSWEYPDTWSTPHSYFSLTFCVQVQGKSKREKKDRVFTDKTS |
| | 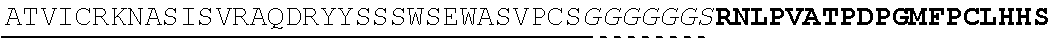 |
| | QNLLRAVSNMLQKARQTLEFYPCTSEEIDHEDITKDKTSTVEACLPLELTKNESCLNSR |
| | ETSFITNGSCLASRKTSFMMALCLSSIYEDLKMYQVEFKTMNAKLLMDPKRQIFLDQNM |
| | LAVIDELMQALNFNSETVPQKSSLEEPDFYKTKIKLCILLHAFRIRAVTIDRVMSYLNA |
| | 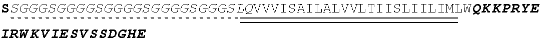 |
| | Italic: signal peptide; Underline: IL12B; Dashed underline |
| | and Italic: linker; Bold: IL12A; Double underline: |
| | Transmembrane domain; Bold and Italic: Intracellular domain |
| | h12AB-PTM-ICD-E570tr Amino Acid Sequence |
| 67 | AUGUGCCACCAGCAGCUGGUGAUCAGCUGGUUCAGCCUGGUGUUCCUGGCCAGCCCCCU |
| | GGUGGCCAUCUGGGAGCUGAAGAAGGACGUGUACGUGGUGGAGUUGGAUUGGUACCCCG |
| | ACGCCCCCGGCGAGAUGGUGGUGCUGACCUGCGACACCCCCGAGGAGGACGGCAUCACC |
| | UGGACCCUGGACCAGAGCAGCGAGGUGCUGGGCAGCGGCAAGACCCUGACCAUCCAGGU |
| | GAAGGAGUUCGGCGACGCCGGCCAGUACACCUGCCACAAGGGCGGCGAGGUGCUGAGCC |
| | ACAGCCUGCUGCUGCUGCACAAGAAGGAGGACGGCAUCUGGAGCACCGACAUCCUGAAG |
| | GACCAGAAGGAGCCCAAGAACAAGACCUUCCUGAGAUGCGAGGCCAAGAACUACAGCGG |
| | CAGAUUCACCUGCUGGUGGCUGACCACCAUCAGCACCGACCUGACCUUCAGCGUGAAGA |
| | GCAGCAGAGGCAGCAGCGACCCCCAGGGCGUGACCUGCGGCGCCGCCACCCUGAGCGCC |
| | GAGAGAGUGAGAGGCGACAACAAGGAGUACGAGUACAGCGUGGAGUGCCAGGAAGAUAG |
| | CGCCUGCCCCGCCGCCGAGGAGAGCCUGCCCAUCGAGGUGAUGGUGGACGCCGUGCACA |
| | AGCUGAAGUACGAGAACUACACCAGCAGCUUCUUCAUCAGAGAUAUCAUCAAGCCCGAC |
| | CCCCCCAAGAACCUGCAGCUGAAGCCCCUGAAGAACAGCCGGCAGGUGGAGGUGAGCUG |
| | GGAGUACCCCGACACCUGGAGCACCCCCCACAGCUACUUCAGCCUGACCUUCUGCGUGC |
| | AGGUGCAGGGCAAGAGCAAGAGAGAGAAGAAAGAUAGAGUGUUCACCGACAAGACCAGC |
| | GCCACCGUGAUCUGCAGAAAGAACGCCAGCAUCAGCGUGAGAGCCCAAGAUAGAUACUA |
| | CAGCAGCAGCUGGAGCGAGUGGGCCAGCGUGCCCUGCAGCGGCGGCGGCGGCGGCGGCA |
| | GCAGAAACCUGCCCGUGGCCACCCCCGACCCCGGCAUGUUCCCCUGCCUGCACCACAGC |
| | CAGAACCUGCUGAGAGCCGUGAGCAACAUGCUGCAGAAGGCCCGGCAGACCCUGGAGUU |
| | CUACCCCUGCACCAGCGAGGAGAUCGACCACGAAGAUAUCACCAAAGAUAAGACCAGCA |
| | CCGUGGAGGCCUGCCUGCCCCUGGAGCUGACCAAGAACGAGAGCUGCCUGAACAGCAGA |
| | GAGACCAGCUUCAUCACCAACGGCAGCUGCCUGGCCAGCAGAAAGACCAGCUUCAUGAU |
| | GGCCCUGUGCCUGAGCAGCAUCUACGAGGACCUGAAGAUGUACCAGGUGGAGUUCAAGA |
| | CCAUGAACGCCAAGCUGCUGAUGGACCCCAAGCGGCAGAUCUUCCUGGACCAGAACAUG |
| | CUGGCCGUGAUCGACGAGCUGAUGCAGGCCCUGAACUUCAACAGCGAGACCGUGCCCCA |
| | GAAGAGCAGCCUGGAGGAGCCCGACUUCUACAAGACCAAGAUCAAGCUGUGCAUCCUGC |
| | UGCACGCCUUCAGAAUCAGAGCCGUGACCAUCGACAGAGUGAUGAGCUACCUGAACGCC |
| | AGCUCUGGUGGCGGAUCAGGCGGCGGCGGUUCAGGAGGCGGUGGAAGUGGAGGUGGCGG |
| | GUCUGGCGGAGGUUCACUGCAGGUGGUGGUGAUCAGCGCCAUCCUGGCCCUGGUGGUGC |
| | UCACCAUCAUCAGCCUGAUCAUCCUGAUCAUGCUGUGGCAGAAGAAGCCCAGAUACGAG |
| | AUCCGGUGGAAGGUGAUCGAGAGCGUGAGCAGCGACGGCCACGAGUUCAUCUUCGUGGA |
| | CCCCAUGCAGCUGCCCUACGACAGCACCUGGGAGCUGCCCCGUGAUCAGCUGGUGCUGG |
| | GCAGAACCCUGGGCAGCGGCGCCUUCGGCCAGGUGGUGGAGGCUACCGCCCACGGCCUG |
| | AGCCACAGCCAGGCCACCAUGAAGGUGGCCGUGGCCAUGCUCAAGAGCACCGCCAGAAG |
| | CAGCGAGAAGCAGGCCCUGAUGAGCGAGCUGAAGAUCAUGAGCCAUCUGGGGCCCCACC |
| | UGAACGUGGUGAACCUGCUGGGCGCCUGCACCAAGGGCGGCCCCAUCUACAUCAUCACC |
| | GAGUACUGCAGAUACGGCGACCUGGUGGACUACCUGCACAGAAACAAGCACACCUUCCU |
| | GCAGCACCACAGCGACAAGAGAAGACCUCCCAGCGCCGAGCUGUACAGCAACGCCCUGC |
| | CCGUUGGUCUGCCCCUACCCAGCCACGUGAGCCUGACCGGCGAGAGCGACGGCGGC |
| | h12AB-PTM-ICD-G739tr Nucleotide Sequence |
| 68 | MCHQQLVISWFSLVFLASPLVAIWELKKDVYVVELDWYPDAPGEMVVLTCDTPEEDGIT |
| | WTLDQSSEVLGSGKTLTIQVKEFGDAGQYTCHKGGEVLSHSLLLLHKKEDGIWSTDILK |
| | DQKEPKNKTFLRCEAKNYSGRFTCWWLTTISTDLTFSVKSSRGSSDPQGVTCGAATLSA |
| | ERVRGDNKEYEYSVECQEDSACPAAEESLPIEVMVDAVHKLKYENYTSSFFIRDIIKPD |
| | PPKNLQLKPLKNSRQVEVSWEYPDTWSTPHSYFSLTFCVQVQGKSKREKKDRVFTDKTS |
| | 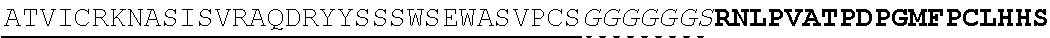 |
| | QNLLRAVSNMLQKARQTLEFYPCTSEEIDHEDITKDKTSTVEACLPLELTKNESCLNSR |
| | ETSFITNGSCLASRKTSFMMALCLSSIYEDLKMYQVEFKTMNAKLLMDPKRQIFLDQNM |
| | LAVIDELMQALNFNSETVPQKSSLEEPDFYKTKIKLCILLHAFRIRAVTIDRVMSYLNA |
| | 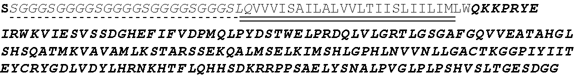 |
| | Italic: signal peptide; Underline: IL12B; Dashed underline |
| | and Italic: linker; Bold: IL12A; Double underline: |
| | Transmembrane domain; Bold and Italic: Intracellular domain |
| | h12AB-PTM-ICD-G739tr Amino Acid Sequence |
| 69 | AUCUACAUCUGGGCUCCACUGGCCGGCACCUGCGGCGUGCUGCUGCUGAGCCUGGUGAU |
| | CACCCUGUACUGCUAC |
| | Human CD8 transmembrane nucleotide sequence |
| 70 | CUGCUGCCCAGCUGGGCCAUCACCCUGAUCAGCGUGAACGGCAUCUUCGUGAUCUGCUG |
| | CCUG |
| | Human CD80 transmembrane domain nucleotide sequence |
| 71 | ACCUACUGCUUCGCCCCUCGAUGCAGAGAGAGAAGAAGAAACGAGAGACUGAGAAGAGA |
| | GAGCGUGCGACCCGUG |
| | Human CD80 intracellular domain nucleotide sequence |
| 72 | CCUUAGCAGAGCUGUGGAGUGUGACAAUGGUGUUUGUGUCUAAACUAUCAAACGCCAUU |
| | AUCACACUAAAUAGCUACUGCUAGGC |
| | (miR-122) |
| 73 | AACGCCAUUAUCACACUAAAUA |
| | (miR-122-3p) |
| 74 | UAUUUAGUGUGAUAAUGGCGUU (miR-122-3p binding site) |
| 75 | UCAAGCUUUUGGACCCUCGUACAGAAGCUAAUACGACUCACUAUAGGGAAAUAAGAGAG |
| | AAAAGAAGAGUAAGAAGAAAUAUAAGAGCCACC |
| | (5′ UTR) |
| 76 | GGGAAAUAAGAGAGAAAAGAAGAGUAAGAAGAAAUAUAAGAGCCACC |
| | (5′ UTR) |
| 77 | UGAUAAUAGGCUGGAGCCUCGGUGGCCAUGCUUCUUGCCCCUUGGGCCUCCCCCCAGCC |
| | CCUCCUCCCCUUCCUGCACCCGUACCCCCCAAACACCAUUGUCACACUCCAGUGGUCUU |
| | UGAAUAAAGUCUGAGUGGGCGGC (3′ UTR) |
| 78 | UGAUAAUAGGCUGGAGCCUCGGUGGCCAUGCUUCUUGCCCCUUGGGCCCAAACACCAUU |
| | GUCACACUCCAUCCCCCCAGCCCCUCCUCCCCUUCCUCCAUAAAGUAGGAAACACUACA |
| | UGCACCCGUACCCCCGUGGUCUUUGAAUAAAGUCUGAGUGGGCGGC |
| | (3′ UTR with mi-122 and mi-142.3p sites) |
| 79 | GSGATNFSLLKQAGDVEENPGP |
| | (2A peptide amino acid sequence) |
| 80 | GGAAGCGGAGCTACTAACTTCAGCCTGCTGAAGCAGGCTGGAGACGTGGAGGAGAACCC |
| | TGGACCT |
| | (Nucleotide sequence encoding 2A peptide) |
| 81 | TCCGGACTCAGATCCGGGGATCTCAAAATTGTCGCTCCTGTCAAACAAACTCTTAACTT |
| | TGATTTACTCAAACTGGCTGGGGATGTAGAAAGCAATCCAGGTCCACTC |
| | (Nucleotide sequence encoding 2A peptide) |
| 82 | UGGAGUGUGACAAUGGUGUUUG |
| | (miR-122-5p) |
| 83 | CAAACACCAUUGUCACACUCCA (miR-122-5p binding site) |
| 84 | UAGCUUAUCAGACUGAUGUUGA (has-miR-21-5p) |
| 85 | CAACACCAGUCGAUGGGCUGU (has-miR-21-3p) |
| 86 | (GGGGS)ₒ o = 1-5 |
| 87 | GGSGGGGSGG |
| 88 | GGSGGGGG |
| 89 | GSGSGSGS |
| 90 | GGGGGGS |
| 91 | GGGGG |
| 92 | GGGGGG |
| 93 | GGGGGGG |
| 94 | GGGGGGGG |
| 95 | (EAAAK)_{q} q = 1-5 |
| 96 | EAAAKEAAAKEAAAK |
| 97 | GGGGSLVPRGSGGGGS |
| 98 | GSGSGS |
| 99 | GGGGSLVPRGSGGGG |
| 100 | GGSGGHMGSGG |
| 101 | GGSGGSGGSGG |
| 102 | GGSGG |
| 103 | GSGSGSGS |
| 104 | GGGSEGGGSEGGGSEGGG |
| 105 | AAGAATAA |
| 106 | GGSSG |
| 107 | GSGGGTGGGSG |
| 108 | GSGSGSGSGGSG |
| 109 | GSGGSGSGGSGGSG |
| 110 | GSGGSGGSGGSGGS |
| 111 | CCGCCGCCGCCG |
| | [CCG]₄ |
| 112 | CCGCCGCCGCCGCCG |
| | [CCG]₅ |
| 113 | CCCCGGCGCC |
| | V1 GC-rich RNA element |
| 114 | CCCCGGC |
| | V2 GC-rich RNA element |
| 115 | GCCGCC |
| | EK GC-rich RNA element |
| 116 | GGGAAATAAGAGAGAAAAGAAGAGTAAGAAGAAATATAAGA |
| | 5′UTR |
| 117 | GGGAAATAAGAGAGAAAAGAAGAGTAAGAAGAAATATAAGACCCCGGCGCCGCCACC |
| | V1-5′UTR |
| 118 | GGGAAATAAGAGAGAAAAGAAGAGTAAGAAGAAATATAAGACCCCGGCGCCACC |
| | V2-5′UTR |
| 119 | GGGAAATAAGAGAGAAAAGAAGAGTAAGAAGAAATATAAGAGCCACC |
| | Standard UTR |
| 120 | GCCA/GCC |
| | Kozak Consensus |
| 121 | UGAUAAUAGGCUGGAGCCUCGGUGGCCUAGCUUCUUGCCCCUUGGGCCUCCCCCCAGCC |
| | CCUCCUCCCCUUCCUGCACCCGUACCCCCCAAACACCAUUGUCACACUCCAGUGGUCUU |
| | UGAAUAAAGUCUGAGUGGGCGGC |
| | 3′UTR with miR-122 |
| 122 | AUGGACGCAAUGAAGAGAGGGCUCUGCUGUGUGCUCCUUCUUUGCGGAGCAGUCUUCGU |
| | UUCGCCCAGCCAGGAAAUCCACGCCCGAUUCAGAAGAGGAGCCAGAAACUGGGUGAACG |
| | UGAUCUCGGACCUUAAGAAGAUCGAGGACCUCAUCCAGUCGAUGCACAUCGACGCGACG |
| | CUGUACACGGAGUCGGACGUCCACCCGUCGUGCAAGGUCACGGCGAUGAAGUGCUUCCU |
| | CCUGGAGCUCCAAGUCAUCUCGCUCGAGAGCGGUGACGCGUCGAUCCACGACACGGUGG |
| | AGAACCUGAUCAUCCUGGCGAACAACUCGCUGUCGUCGAACGGGAACGUCACAGAGUCC |
| | GGCUGCAAGGAGUGCGAGGAGCUGGAGGAGAAGAACAUCAAGGAGUUCCUGCAGUCGUU |
| | CGUUCAUAUAGUCCAGAUGUUCAUCAACACGUCG |
| | HS IL15opt-tPA6 mRNA ORF #2 |
| 123 | MDAMKRGLCCVLLLCGAVFVSPSQEIHARFRRGARNWVNVISDLKKIEDLIQSMHIDAT |
| | LYTESDVHPSCKVTAMKCFLLELQVISLESGDASIHDTVENLIILANNSLSSNGNVTES |
| | 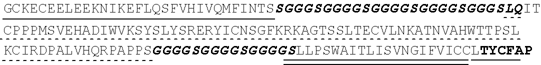 |
| | RCRERRRNERLRRESVRPV |
| | Hs tPA6-ILR_linker_CD80TID (signal peptide is bold; IL-15 |
| | is underlined; linkers are bold and italic; IL-15Ra sushi |
| | domain is dotted underlined; CD80 transmembrane domain is |
| | double underlined; CD80 intracellular domain is bold and |
| | underlined) |
| 124 | ATGGATGCAATGAAGAGAGGGCTCTGCTGTGTGCTGCTGCTGTGTGGAGCAGTCTTCGT |
| | TTCGCCCAGCCAGGAAATCCATGCCCGATTCAGAAGAGGAGCCAGAAACTGGGTGAACG |
| | TGATCTCGGACCTGAAGAAGATCGAGGACCTCATCCAGTCGATGCACATCGACGCGACG |
| | CTGTACACGGAGTCGGACGTCCACCCGTCGTGCAAGGTCACGGCGATGAAGTGCTTCCT |
| | CCTGGAGCTCCAAGTCATCTCGCTCGAGTCGGGCGACGCGTCGATCCACGACACGGTGG |
| | AGAACCTGATCATCCTGGCGAACAACTCGCTGTCGTCGAACGGGAACGTCACGGAGTCG |
| | GGCTGCAAGGAGTGCGAGGAGCTGGAGGAGAAGAACATCAAGGAGTTCCTGCAGTCGTT |
| | CGTGCACATCGTCCAGATGTTCATCAACACGTCGTCTGGTGGCGGATCAGGCGGTGGCG |
| | GATCTGGCGGCGGTGGAAGTGGAGGTGGCGGGTCTGGCGGAGGTTCACTGCAGATCACC |
| | TGTCCACCTCCCATGTCCGTGGAACACGCCGACATCTGGGTCAAGAGCTACAGCCTGTA |
| | CTCCCGGGAAAGATACATTTGTAACAGCGGGTTTAAGAGGAAGGCCGGCACCTCCTCGT |
| | TGACCGAATGCGTGCTGAACAAGGCTACCAACGTGGCCCATTGGACTACCCCGTCCCTG |
| | AAGTGCATTCGCGATCCTGCCCTCGTGCACCAACGGCCTGCGCCGCCGTCCGGCGGCGG |
| | CGGTTCAGGCGGAGGAGGAAGTGGAGGTGGAGGCTCCCTGCTGCCCAGCTGGGCCATCA |
| | CCCTGATCAGCGTGAACGGCATCTTCGTGATCTGCTGCCTGACCTACTGCTTCGCTCCC |
| | AGATGCAGAGAGAGAAGAAGAAACGAGAGACTGAGAAGAGAGAGCGTGAGACCCGTG |
| | Nucleotide Sequence #1: Hs tPA6-ILR_linker_CD80TID |
| 125 | ATGGATGCAATGAAGAGAGGGCTCTGCTGTGTGCTGCTGCTGTGTGGAGCAGTCTTCGT |
| | TTCGCCCAGCCAGGAAATCCATGCCCGATTCAGAAGAGGAGCCAGAAACTGGGTGAACG |
| | TGATCTCGGACCTGAAGAAGATCGAGGACCTCATCCAGTCGATGCACATCGACGCGACG |
| | CTGTACACGGAGTCGGACGTCCACCCGTCGTGCAAGGTCACGGCGATGAAGTGCTTCCT |
| | CCTGGAGCTCCAAGTCATCTCGCTCGAGTCGGGCGACGCGTCGATCCACGACACGGTGG |
| | AGAACCTGATCATCCTGGCGAACAACTCGCTGTCGTCGAACGGGAACGTCACGGAGTCG |
| | GGCTGCAAGGAGTGCGAGGAGCTGGAGGAGAAGAACATCAAGGAGTTCCTGCAGTCGTT |
| | CGTGCACATCGTCCAGATGTTCATCAACACGTCGAGCGGCGGCGGTAGTGGAGGAGGTG |
| | GCTCCGGTGGAGGCGGCAGCGGTGGCGGTGGAAGTGGCGGCGGCAGCCTGCAGATCACC |
| | TGTCCTCCTCCCATGAGCGTGGAGCACGCCGACATCTGGGTGAAGAGCTACAGCCTGTA |
| | CAGCCGGGAGCGGTACATCTGCAACAGCGGCTTCAAGCGGAAGGCCGGCACAAGCAGCC |
| | TGACCGAGTGCGTGCTGAACAAGGCCACCAACGTGGCCCACTGGACCACCCCAAGCCTG |
| | AAGTGCATCCGGGACCCCGCCCTGGTGCATCAGCGGCCCGCCCCACCTAGCGGCGGAGG |
| | AGGCTCCGGAGGAGGCGGGAGCGGTGGAGGCGGCAGCCTGCTGCCCTCTTGGGCCATCA |
| | CCCTGATCAGCGTGAACGGCATCTTCGTGATCTGCTGCCTGACCTACTGCTTTGCCCCT |
| | AGGTGCCGGGAGAGACGGCGGAACGAGAGGCTGCGGCGGGAGAGCGTGCGGCCCGTG |
| | Nucleotide Sequence #2: Hs tPA6-ILR_GS_CD80TID-partopt |
| 126 | ATGGACGCCATGAAGCGGGGCCTGTGCTGCGTGCTGCTGCTGTGCGGCGCCGTGTTCGT |
| | GAGCCCCAGCCAGGAGATCCACGCCCGGTTTAGACGGGGCGCACGGAACTGGGTGAACG |
| | TGATCAGCGACCTGAAGAAGATCGAGGACCTGATCCAGAGCATGCACATCGACGCCACC |
| | CTGTACACCGAGAGCGACGTGCACCCCAGCTGCAAGGTGACCGCCATGAAGTGCTTCCT |
| | GCTGGAGCTGCAGGTGATCAGCCTGGAGAGCGGCGACGCCAGCATCCACGACACCGTGG |
| | AGAACCTGATCATCCTGGCCAACAACAGCCTGAGCAGCAACGGCAACGTGACCGAGAGC |
| | GGCTGCAAGGAGTGCGAGGAGCTGGAGGAGAAGAACATCAAGGAGTTCCTGCAGAGCTT |
| | CGTGCACATCGTGCAGATGTTCATCAACACCAGCTCTGGTGGCGGCTCCGGTGGAGGTG |
| | GCTCTGGCGGCGGAGGTAGCGGTGGCGGAGGCAGTGGCGGAGGGAGCCTGCAGATCACC |
| | TGCCCACCACCCATGAGCGTGGAGCACGCCGACATCTGGGTGAAGAGCTACAGCCTGTA |
| | CAGCCGGGAGCGGTACATCTGCAACAGCGGCTTCAAGCGGAAGGCCGGCACCAGCAGCC |
| | TGACCGAGTGCGTGCTGAACAAGGCCACCAACGTGGCCCACTGGACCACCCCGAGCCTG |
| | AAGTGCATCCGGGACCCAGCCCTGGTGCATCAGCGGCCCGCACCTCCAAGCGGCGGAGG |
| | CGGAAGTGGCGGCGGCGGTTCAGGCGGAGGAGGCAGCCTCCTGCCCTCTTGGGCCATCA |
| | CCCTGATCAGCGTGAACGGCATCTTCGTGATCTGCTGCCTGACCTACTGCTTCGCACCC |
| | CGGTGCAGAGAGCGGCGGCGGAACGAGCGCCTGCGGCGAGAGAGCGTGCGGCCCGTG |
| | Nucleotide Sequence #3: Hs tPA6-ILR_GS_CD80TID_fullopt |
| 127 | UGUAGUGUUUCCUACUUUAUGGA (hsa-miR-142-3p) |
| 128 | CAUAAAGUAGAAAGCACUACU (hsa-miR-142-5p) |
| 129 | ITCPPPMSVEHADIWVKSYSLYSRERYICNSGFKRKAGTSSLTECVLNKATNVAHWTTP |
| | SLKCIRDPALVHQRPAPPS |
| | IL-15Ra sushi domain |
| 130 | VAISTSTVLLCGLSAVSLLACYL |
| | IL-15Ra transmembrane domain |
| 131 | KSRQTPPLASVEMEAMEALPVTWGTSSRDEDLENCSHHL |
| | IL-15Ra intracellular domain |
| 132 | GGGAAAUAAGAGAGAAAAGAAGAGUAAGAAGAAAUAUAAGA |
| | 5′UTR |
| 133 | GGGAAAUAAGAGAGAAAAGAAGAGUAAGAAGAAAUAUAAGACCCCGGCGCCGCCACC |
| | V1-5′UTR |
| 134 | GGGAAAUAAGAGAGAAAAGAAGAGUAAGAAGAAAUAUAAGACCCCGGCGCCACC |
| | V2-5′UTR |
| 135 | CCR(A/G)CCAUGG |
| | Kozak consensus sequence |
| 136 | GGGAGAUCAGAGAGAAAAGAAGAGUAAGAAGAAAUAUAAGAGCCACC |
| | 5′UTR-002 |
| 137 | GGGAGACAAGCUUGGCAUUCCGGUACUGUUGGUAAAGCCACC |
| | 5′UTR-003 |
| 138 | GGGAAUUAACAGAGAAAAGAAGAGUAAGAAGAAAUAUAAGAGCCACC |
| | 5′UTR-008 |
| 139 | GGGAAAUUAGACAGAAAAGAAGAGUAAGAAGAAAUAUAAGAGCCACC |
| | 5′UTR-009 |
| 140 | GGGAAAUAAGAGAGUAAAGAACAGUAAGAAGAAAUAUAAGAGCCACC |
| | 5′UTR-010 |
| 141 | GGGAAAAAAGAGAGAAAAGAAGACUAAGAAGAAAUAUAAGAGCCACC |
| | 5′UTR-011 |
| 142 | GGGAAAUAAGAGAGAAAAGAAGAGUAAGAAGAUAUAUAAGAGCCACC |
| | 5′UTR-012 |
| 143 | GGGAAAUAAGAGACAAAACAAGAGUAAGAAGAAAUAUAAGAGCCACC |
| | 5′UTR-013 |
| 144 | GGGAAAUUAGAGAGUAAAGAACAGUAAGUAGAAUUAAAAGAGCCACC |
| | 5′UTR-014 |
| 145 | GGGAAAUAAGAGAGAAUAGAAGAGUAAGAAGAAAUAUAAGAGCCACC |
| | 5′UTR-015 |
| 146 | GGGAAAUAAGAGAGAAAAGAAGAGUAAGAAGAAAAUUAAGAGCCACC |
| | 5U′TR-016 |
| 147 | GGGAAAUAAGAGAGAAAAGAAGAGUAAGAAGAAAUUUAAGAGCCACC |
| | 5′UTR-017 |

1. A method of treating a cancer in a human patient, comprising administering to the patient:
(i) a first mRNA encoding human OX40L; and
(ii) at least one second mRNA encoding an immune potentiator, wherein the immune potentiator is a cell-associated cytokine that activates T cells, NK cells, or both T cells and NK cells,
wherein the first mRNA and at least one second mRNA are encapsulated in the same or different lipid nanoparticles.

2. The method of claim 1, wherein the cancer is a disseminated cancer and wherein the first mRNA and the at least one second mRNA are administered systemically.

3. The method of claim 2, wherein the disseminated cancer is a hematological cancer or is a myeloid malignancy.

4. (canceled)

5. The method of claim 3, wherein the myeloid malignancy is selected from the group consisting of myeloidysplastic syndrome (MDS), myeloproliferative disorder (MPD) and acute myeloid leukemia (AML).

6. The method of claim 1, wherein the cancer is a solid tumor and wherein the first mRNA and the at least one second mRNA are administered intratumorally.

7.-24. (canceled)

25. The method of claim 1, comprising:
(i) a first fractionated dose of a pharmaceutical composition comprising the first mRNA, and the at least one second mRNA, and
(ii) at least one second fractionated dose of the pharmaceutical composition, wherein the first and second fractionated doses increase exposure to the mRNA encoded polypeptides in the patient relative to a single dose of the same amount of mRNA during the same dosing interval, thereby treating the disseminated cancer in the patient.

26. The method of claim 25, wherein the first fractionated dose and second fractionated dose enhance (i) anti-tumor efficacy of the treatment relative to a single dose of the same amount of mRNA, (ii) enhance anti-tumor efficacy with reduced toxicity and better tolerability, or (i) and (ii).

27.-52. (canceled)

53. The method of claim 1, comprising administering a checkpoint inhibitor polypeptide, wherein the checkpoint inhibitor polypeptide inhibits PD-1, PD-L¹, CTLA-4, or a combination thereof.

54. The method of claim 53, wherein the checkpoint inhibitor polypeptide is an antibody or an mRNA encoding the antibody.

55.-56. (canceled)

57. A lipid nanoparticle comprising:
(i) a first mRNA encoding human OX40L; and
(ii) at least one second mRNA encoding an immune potentiator, wherein the immune potentiator is a cell-associated cytokine that activates T cells, NK cells, or both T cells and NK cells.

58. A lipid nanoparticle comprising:
(i) an ionizable lipid;
(ii) a sterol or other structural lipid;
(iii) a first mRNA encoding human OX40;
(iv) at least one second mRNA encoding an immune potentiator, wherein the immune potentiator is a cell-associated cytokine that activates T cells, NK cells, or both T cells and NK cells;
(v) optionally, a non-cationic helper lipid or phospholipid; and
(vi) optionally, a PEG-lipid.

59. The lipid nanoparticle of claim 57, wherein the at least one second mRNA is:
(i) an mRNA encoding a trans-presented human IL-15;
(ii) an mRNA encoding a human IL-12 polypeptide operably linked to a membrane domain comprising a transmembrane domain; or
(iii) an mRNA encoding a trans-presented human IL-15 and an mRNA encoding a human IL-12 polypeptide operably linked to a membrane domain.

60. The lipid nanoparticle of claim 59, wherein the trans-presented human IL-15 (i) is a human IL-15 polypeptide operably linked to a human IL-15Rα polypeptide; or (ii) is encoded by a first mRNA encoding a human IL-15 polypeptide and a second mRNA encoding a human IL-15Rα polypeptide.

61.-62. (canceled)

63. The lipid nanoparticle of claim 59, wherein the IL-12 polypeptide comprises an IL-12 p40 subunit (IL-12B) polypeptide operably linked, optionally via a peptide linker, to an IL-12 p35 subunit (IL-12A) polypeptide.

64. The lipid nanoparticle of claim 63, wherein the IL-12B polypeptide is located at the 5′ terminus of the IL-12A polypeptide, or the 5′ terminus of the peptide linker; or wherein the IL-12A polypeptide is located at the 5′ terminus of the IL-12B polypeptide, or the 5′ terminus of the peptide linker.

65. The lipid nanoparticle of claim 59, wherein: (i) the IL-12 polypeptide transmembrane domain comprises a transmembrane domain derived from a Type I integral membrane protein; or (ii) the IL-12 polypeptide transmembrane domain is selected from the group consisting of: a Cluster of Differentiation 8 (CD8) transmembrane domain, a Platelet-Derived Growth Factor Receptor (PDGFR) transmembrane domain, and a Cluster of Differentiation 80 (CD80) transmembrane domain.

66. (canceled)

67. The lipid nanoparticle of claim 59, wherein the IL-12 polypeptide membrane domain comprises an intracellular domain, wherein (i) the intracellular domain is derived from the same polypeptide as the transmembrane domain, or wherein the intracellular domain is derived from a different polypeptide than the transmembrane domain is derived from; or (ii) the intracellular domain is selected from the group consisting of: a PDGFR intracellular domain, a truncated PDGFR intracellular domain, and a CD80 intracellular domain.

68.-69. (canceled)

70. The lipid nanoparticle of claim 59, wherein the IL-12 polypeptide membrane domain comprises:
(i) a PDGFR-beta transmembrane domain and a PDGFR-beta intracellular domain;
(ii) a PDGFR-beta transmembrane domain and a truncated PDGFR-beta intracellular domain truncated at E570;
(iii) a PDGFR-beta transmembrane domain and a truncated PDGFR-beta intracellular domain truncated at G739; or
(iv) a CD80 transmembrane domain and a CD80 intracellular domain.

71. The lipid nanoparticle of claim 59, wherein the IL-12 polypeptide membrane domain is operably linked to the IL-12A polypeptide by a peptide linker, or wherein the IL-12 polypeptide membrane domain is operably linked to the IL-12B polypeptide by a peptide linker.

72. The lipid nanoparticle of claim 59, wherein the IL-15Rα polypeptide comprises a sushi domain.

73. The lipid nanoparticle of claim 72, wherein the IL-15Rα polypeptide further comprises an intracellular domain and a transmembrane domain, and wherein the intracellular domain and the transmembrane domain are derived from IL-15Rα or from a heterologous polypeptide.

74.-76. (canceled)

77. The lipid nanoparticle of claim 57, wherein each mRNA comprises a 3′ untranslated region (UTR) and a 5′UTR.

78. The lipid nanoparticle of claim 77, wherein the 3′UTR comprises at least one microRNA (miR) binding site, wherein the at least one miR binding site is a miR-122 binding site, and wherein the miR-122 binding site is a miR-122-3p or a miR-122-5p binding site.

79.-82. (canceled)

83. The lipid nanoparticle of claim 57, wherein each mRNA includes at least one chemical modification.

84. (canceled)

85. The lipid nanoparticle of claim 57, wherein (i) at least 95% of uridines in each mRNA are N1-methylpseudouridine; (ii) at least 99% of uridines in each mRNA are N1-methylpseudouridine; or (iii) 100% of uridines in each mRNA are N1-methylpseudouridine.

86. The lipid nanoparticle of claim 57, wherein the lipid nanoparticle comprises a molar ratio of about 20-60% ionizable amino lipid: 5-25% phospholipid: 25-55% structural lipid; and 0.5-15% PEG-modified lipid.

87.-89. (canceled)

90. The lipid nanoparticle of claim 86, wherein the ionizable lipid comprises Compound X.

91. The lipid nanoparticle of claim 57, wherein the lipid nanoparticle comprises a molar ratio of about 20-60% Compound X: 5-25% phospholipid: 25-55% cholesterol; and 0.5-15% PEG-modified lipid.

92. (canceled)

93. The lipid nanoparticle of claim 86, wherein the PEG-modified lipid is PEG-DMG or Compound P-428.

94. The lipid nanoparticle of claim 86, wherein the lipid nanoparticle comprises a phytosterol or a combination of a phytosterol and cholesterol.

95. The lipid nanoparticle of claim 94, wherein the phytosterol (i) is selected from the group consisting of β-sitosterol, stigmasterol, β-sitostanol, campesterol, brassicasterol, and combinations thereof; (ii) comprises a sitosterol or a salt or an ester thereof;

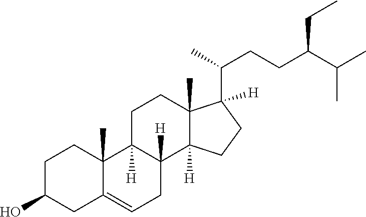

96.-97. (canceled)

98. The lipid nanoparticle of claim 57, wherein the lipid nanoparticle comprises a molar ratio of 40:38.5:20:1.5 of Compound X:cholesterol:phospholipid:Compound P-428, or of Compound X:cholesterol:DSPC:Compound P-428.

99. The lipid nanoparticle of claim 94, wherein the mol % sterol or other structural lipid is (i) 18.5% phytosterol and the total mol % structural lipid is 38.5%, or (ii) 28.5% phytosterol and the total mol % structural lipid is 38.5%.

100.-102. (canceled)

103. The lipid nanoparticle of claim 57, formulated for intratumoral delivery.

104. The lipid nanoparticle of claim 57, formulated for intravenous delivery.

105.-106. (canceled)
